(19) United States
(12) Patent Application Publication
(10) Pub. No.: US 2026/0124226 A1
(43) Pub. Date: May 7, 2026

(54) 4'-HALOGEN CONTAINING NUCLEOTIDE AND NUCLEOSIDE THERAPEUTIC COMPOSITIONS AND USES RELATED THERETO

(71) Applicant: EMORY UNIVERSITY, Atlanta, GA (US)

(72) Inventors: George R. PAINTER, Atlanta, GA (US); Shuli MAO, Marietta, GA (US); Michael G. NATCHUS, Alpharetta, GA (US); Damien KUIPER, Atlanta, GA (US); David PERRYMAN, Decatur, GA (US)

(21) Appl. No.: 19/380,466

(22) Filed: Nov. 5, 2025

Related U.S. Application Data
(63) Continuation of application No. PCT/US2024/027749, filed on May 3, 2024.
(60) Provisional application No. 63/500,416, filed on May 5, 2023.
(60) Provisional application No. 63/534,748, filed on Aug. 25, 2023.
(60) Provisional application No. 63/588,255, filed on Oct. 5, 2023.
(60) Provisional application No. 63/571,795, filed on Mar. 29, 2024.

(51) Int. Cl. A61K 31/7072; A61P 31/14; A61P 31/16; C07H 19/067

(52) U.S. Cl. CPC A61K 31/7072; A61P 31/14; A61P 31/16; C07H 19/067

(57) ABSTRACT

Disclosed are halogen containing nucleotide and nucleoside therapeutic compositions and uses related thereto. In certain embodiments, the disclosure relates to the treatment or prophylaxis of viral infections. Such viral infections can include enterovirus, tongaviridae, bunyaviridae, arenaviridae, coronaviridae, flaviviridae, picornaviridae, Eastern, Western, and Venezuelan Equine Encephalitis (EEE, WEE and VEE, respectively), Chikungunya fever (CHIK), Ebola, Influenza, RSV, and Zika virus infections.

### CROSS-REFERENCE TO RELATED APPLICATIONS

This application is a continuation of International Patent Application No. PCT/US2024/027749, filed May 3, 2024, which claims priority to U.S. Provisional Application No. 63/500,416, filed May 5, 2023; U.S. Provisional Application No. 63/534,748, filed Aug. 25, 2023; U.S. Provisional Application No. 63/588,255, filed Oct. 5, 2023; and U.S. Provisional Application No. 63/571,795, filed Mar. 29, 2024; the disclosures of which are incorporated herein by reference.

### STATEMENT ACKNOWLEDGING GOVERNMENT SUPPORT

This invention was made with government support under Grant No. W15QKN-16-9-1002 awarded by the Department of Defense and Grant No. AI171403 awarded by the National Institutes of Health. The government has certain rights in the invention.

### FIELD

This disclosure relates to halogen containing nucleotide and nucleoside therapeutic compositions and uses related thereto. In certain embodiments, the disclosure relates to the treatment or prophylaxis of viral infections, for example, respiratory viruses, enteroviruses, tongaviridae, bunyaviridae, arenaviridae, coronaviridae, flaviviridae, picornaviridae, Eastern, Western, and Venezuelan Equine Encephalitis (EEE, WEE and VEE, respectively), Chikungunya fever (CHIK), Ebola, Influenza, RSV, and Zika virus infections, and conditions caused by these viruses.

### BACKGROUND

New antiviral agents to treat or prevent a variety of viral infections are urgently needed. For example, the causative agents for Eastern, Western, and Venezuelan Equine Encephalitis (EEE, WEE and VEE, respectively) and Chikungunya fever (CHIK) are vector-borne viruses (family Togaviridae, genus Alphavirus) that can be transmitted to humans through mosquito bites. The equine encephalitis viruses are CDC Category B pathogens, and the CHIK virus is Category C. There is considerable concern about the use of virulent strains of VEE virus, delivered via aerosol, as a bioweapon against warfighters. Animal studies have demonstrated that infection with VEE virus by aerosol exposure rapidly leads to a massive infection of the brain, with high mortality and morbidity. See Roy et al., Pathogenesis of aerosolized Eastern equine encephalitis virus infection in guinea pigs. Virol J, 2009, 6:170.

What are needed are new compounds and treatments for viral infections. The compounds and methods disclosed herein addressed these needs.

References cited herein are not an admission of prior art.

### SUMMARY

This disclosure relates to halogen, e.g., 4′-halogen, containing nucleotide and nucleoside therapeutic compositions and uses related thereto. Included are nucleosides optionally conjugated to a phosphorus oxide or salts thereof, prodrugs or conjugate compounds or salts thereof comprising an amino acid ester, lipid or a sphingolipid or derivative linked by a phosphorus oxide to a nucleotide or nucleoside.

In certain embodiments, the disclosure relates to a compound having a structure represented by a formula:

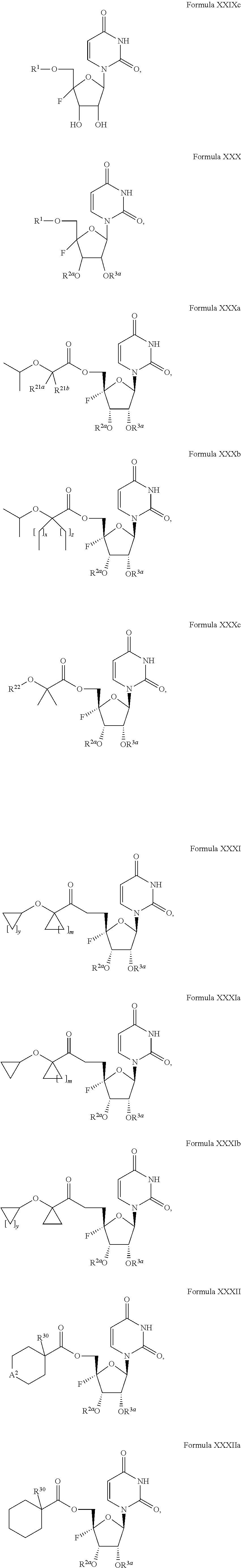

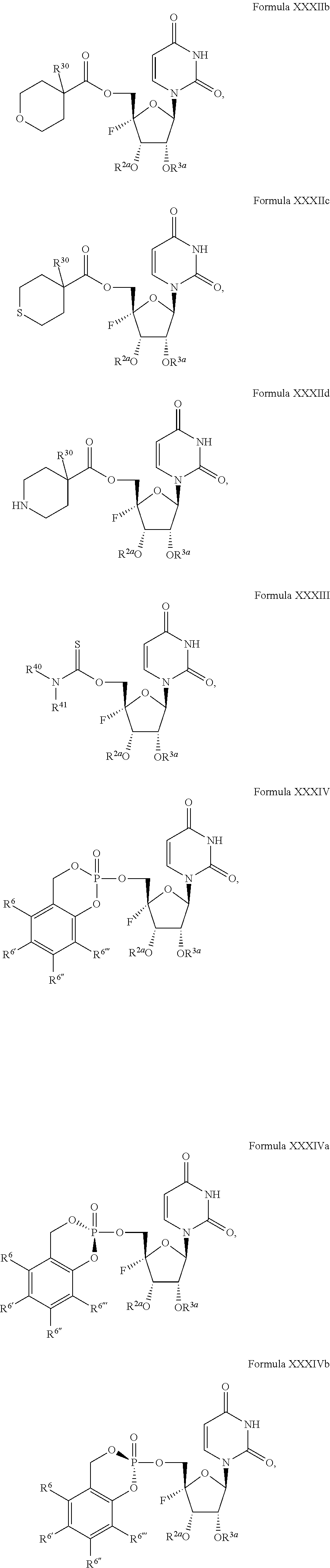

or a subset of the foregoing,
or a pharmaceutically acceptable salt, derivative, or prodrug thereof, as defined herein. In the foregoing formulas, the various substituent groups are understood to have the meaning as further disclosed herein below.

In certain embodiments, the disclosure contemplates derivatives of compounds disclosed herein, such as those containing one or more, the same or different, substituents.

In certain embodiments, the disclosure contemplates pharmaceutical compositions comprising a pharmaceutically acceptable excipient and a compound disclosed herein. In certain embodiments, the pharmaceutical composition is in the form of a tablet, capsule, pill, or aqueous buffer, such as a saline or phosphate buffer.

In certain embodiments, the disclosed pharmaceutical compositions can comprise a compound disclosed herein and a propellant. In certain embodiments, the propellant is an aerosolizing propellant such as compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane or combinations thereof.

In certain embodiments, the disclosure contemplates a pressurized or unpressurized container comprising a compound or pharmaceutical composition as described herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, the disclosure relates to methods of increasing bioavailability for treating or preventing a viral infection comprising administering an effective amount of a compound of Formulas XXIX-XXXIVb, or a pharmaceutical composition comprising a compound of Formulas XXIX-XXXIVb and a pharmaceutically acceptable excipient, disclosed herein to a subject in need thereof.

In certain embodiments, the disclosure relates to methods of treating or preventing a viral infection comprising administering an effective amount of a compound of Formulas XXIX-XXXIVb, or a pharmaceutical composition comprising a compound of Formulas XXIX-XXXIVb and a pharmaceutically acceptable excipient, disclosed herein to a subject in need thereof. In certain embodiments, the viral infection is tongaviridae, bunyaviridae, arenaviridae, coronaviridae, flaviviridae, picornaviridae, Zika virus infection, Eastern, Western, and Venezuelan Equine Encephalitis (EEE, WEE and VEE, respectively), Chikungunya fever (CHIK), Ebola, Influenza, and RSV.

In certain embodiments, the compound or pharmaceutical composition is administered orally, intravenously, or through the lungs, i.e., pulmonary administration.

In certain embodiments, the disclosure relates to the use of a compound as described herein in the production of a medicament for the treatment or prevention of a viral infection, such as Eastern, Western, and Venezuelan Equine Encephalitis (EEE, WEE and VEE, respectively), Chikungunya fever (CHIK), Ebola, Influenza, RSV, or Zika virus infection.

In certain embodiments, the disclosure relates to methods of making compounds disclosed herein by mixing starting materials and reagents disclosed herein under conditions such that the compounds are formed.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

FIG. 1 shows stability of EIDD-2749 in methanol.

FIG. 2 shows stability of EIDD-2749 in water.

FIG. 3 shows stability of EIDD-2749 in 0.1 N HCl.

FIG. 4 shows stability of EIDD-2749 in PBS at pH 7.4.

FIG. 5 shows stability of EIDD-2749 in pH 9 buffer.

FIG. 6 shows triphosphate concentrations in Huh-7 cells incubated with EIDD-2749 or a prodrug.

FIG. 7 shows triphosphate concentrations in Vero cells incubated with EIDD-2749 or a prodrug.

FIG. 8 shows plasma PK curves from CD-1 mice that received a single dose of EIDD-2749 at 50, 150, and 500 mg/kg PO or 10 mg/kg IP.

FIG. 9 shows the change in body weight of AG129 mice dosed with 10, 30, and 100 mg/kg EIDD-2749 QD for 10 days.

FIGS. 10A-10B show representative data for uptake and metabolism of EIDD-3232 in tissue culture experiments compared to EIDD-2749. FIG. 10A shows data for the uptake and metabolism of EIDD-3232 in the indicated cell lines. FIG. 10B shows data for the uptake and metabolism of EIDD-3232 in the indicated cell lines compared to EIDD-2749.

FIGS. 11A-11B show representative data for pharmacokinetics of EIDD-3232, EIDD-3321, and EIDD-2749 in mouse plasma after oral dosing at 10 mg/kg of EIDD-3232 or 5 mg/kg of EIDD-2749. FIG. 11A shows the plasma concentration of the indicated compounds versus time. FIG. 11B shows plasma concentration of EIDD-2749 after dosing with either EIDD-3232 or EIDD-2749 as indicated.

FIGS. 12A-12B show representative data for tissue distribution following dosing with EIDD-3232. FIG. 12A shows the tissue concentration of EIDD-2749 following oral dosing with EIDD-3232. FIG. 12B shows the tissue concentration of EIDD-2991 following oral dosing with EIDD-3232.

FIG. 13 shows representative data for stability of EIDD-3232 in simulated gastric fluid (pH 1.3).

FIG. 14 shows representative data for stability of EIDD-3232 in simulated intestinal fluid (pH 5.2).

FIG. 15 shows the necropsy scoring (gut tissue) and change in body weight of Sprague-Dawley rats dosed as indicated in the figure with the indicated compounds.

FIG. 16 shows the efficacy of the indicated compound (dosed as indicated in the figure) in a mouse survival model of IAV infection.

FIG. 17 shows intracellular concentrations of EIDD-2991 in Calu3 or Huh7 Cells Incubated with EIDD-3639 or EIDD-3640. Results are shown as averages and standard deviations (n=3).

FIG. 18 shows mouse plasma concentrations and pharmacokinetic parameters for EIDD-2749 after a single oral dose of EIDD-3639 or EIDD-3640 at 16.5 mg/kg. Results are shown as averages and standard deviations (n=4).

FIGS. 19A-19B show mouse tissue concentrations of EIDD-2749 (FIG. 19A) and EIDD-2991 (FIG. 19Bl) three hours after a single oral dose of EIDD-3639 or EIDD-3640 at 16.5 mg/kg. Results are shown as averages and standard deviations.

FIG. 20 shows plasma concentrations (nmol/mL) of EIDD-2749 in mouse plasma after a single oral dose of EIDD-2749 at 15 mg/kg, EIDD-3519 at 25 mg/kg, or EIDD-2838 at 25 mg/kg. Results are shown as averages and standard deviations (n=3).

FIG. 21 shows tissue concentrations (nmol/mL) of EIDD-2749 (Nuc; Top Panels) and EIDD-2991 (TP; Bottom Panels) in mouse tissues after a single oral dose of EIDD-2749 at 15 mg/kg, EIDD-3519 at 25 mg/kg, or EIDD-2838 at 25 mg/kg. Black Bars: Dosed with EIDD-2749, Blue Bars: Dosed with EIDD-3519, Red Bars: Dosed with EIDD-2991. Results are shown as averages and standard deviations (n=3).

FIG. 22 shows representative data for stability of EIDD-2749 prodrugs at 10 μM in SGF, pH 1, 37° C. determined using methods described herein below. The prodrug number is shown in the figure and corresponds to the same prodrug number and associated compound structure as disclosed herein below. The calculated t_{1/2} values for EIDD-2839 and EIDD-3509 are shown in the inset adjacent to the figure.

FIGS. 23A-23C show representative data for stability of EIDD-3509 in the presence of mouse intestinal microsomes (FIG. 23A), mouse liver microsome (FIG. 23B) and mouse plasma (FIG. 23C). Drug concentration was determined using assay methods as disclosed herein below, and the stability assays were carried out as described below in the Examples.

FIGS. 24A-24C show representative data for stability of indicated prodrug in the presence of mouse intestinal microsomes. FIG. 24A shows the time course stability of EIDD-3051 and the concomitant release of EIDD-2749. FIG. 24B shows the time course stability of EIDD-3471 and the concomitant release of EIDD-2749. FIG. 24C shows the time course stability of EIDD-3469 and the concomitant release of EIDD-2749. Drug concentration was determined using assay methods as disclosed herein below, and the stability assays were carried out as described below in the Examples.

FIGS. 25A-25B show representative data for stability of indicated prodrug in the presence of mouse intestinal microsomes. FIG. 25A shows the time course stability of EIDD-2838 and the concomitant release of EIDD-2749 and EIDD-2986. FIG. 24B shows the time course stability of EIDD-3519 and the concomitant release of EIDD-2729. Drug concentration was determined using assay methods as disclosed herein below, and the stability assays were carried out as described below in the Examples.

FIGS. 26A-26C show representative for in vivo tolerability of the indicated selected disclosed drugs when dosed with the indicated compounds using a protocol as described in FIG. 27A and Example 140. FIG. 26A shows body weight data collected for the indicated compounds. FIG. 26B shows necroscopy score obtained for the indicated compounds. FIG. 26C shows probability of survivability for the indicated compounds.

FIGS. 27A-27G show representative for in vivo tolerability of the indicated selected disclosed drugs when dosed with the indicated compounds using a protocol as described in FIG. 27A and Example 140. FIG. 27A shows an overall study protocol used to obtain the data shown in FIGS. 26A-26C and 27B-27G. FIG. 27B shows body weight data for the indicated compounds obtained in the foregoing study. FIG. 27C shows necroscopy score obtained for the indicated compounds in the foregoing study. FIG. 27D shows body temperature data obtained for the indicated compounds in the foregoing study. FIG. 27E shows efficacy data obtained for the indicated compounds in an animal model of IAV as described herein below in the Examples. FIG. 27F shows efficacy data obtained for the indicated compounds in an animal model of IAV as described herein below in the Examples. FIG. 27G shows efficacy data obtained for the indicated compounds in an animal model of IAV as described herein below in the Examples.

FIG. 28A shows stability data of EIDD-3621 in simulated gastric fluid using the protocol as described herein below. Data are given in FIG. 28B for release of EIDD-2749 from EIDD-3621 when incubated in the presence of mouse intestinal microsomes, mouse liver microsomes, and mouse plasma using the protocols as described herein below.

FIG. 29 shows the survival of AG129 mice infected with Tacaribe virus and treated with EIDD-02749 starting 2 hours before infection.

FIG. 30 shows Day 9 viral titers in serum (Graph A); liver tissue (Graph B); spleen tissue (Graph C); and brain tissue (Graph D) tissues from AG129 mice infected with Tacaribe virus and treated with EIDD-02749.

FIG. 31 shows the survival of AG129 mice infected with Tacaribe virus treated with EIDD-02749 starting at 2 hours, 1 Day, 3 Days, 5 Days, and 7 Days post infection.

FIG. 32 shows Day 9 viral titers in tissues from AG129 mice infected with Tacaribe virus and treated with EIDD-02749 starting at 2 hours, 1 Day, 3 Days, 5 Days, and 7 Days post infection.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features, which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

This disclosure relates to 4′-halogen containing nucleotide and nucleoside therapeutic compositions and uses related thereto. In certain embodiments, the disclosure relates to nucleosides optionally conjugated to a phosphorus oxide or salts thereof. In certain embodiments, the disclosure relates to conjugate compounds or salts thereof comprising an amino acid ester, a lipid or a sphingolipid or derivative linked by a phosphorus oxide to a nucleotide or nucleoside. In certain embodiments, the disclosure contemplates pharmaceutical compositions comprising these compounds for uses in treating infectious diseases, viral infections, and cancer.

In certain embodiments, the disclosure relates to phosphorus oxide prodrugs of 4′-halogen containing nucleosides for the treatment of positive-sense and negative-sense RNA viral infections through targeting of the virally encoded RNA-dependent RNA polymerase (RdRp). This disclosure also provides the general use of lipids and sphingolipids to deliver nucleoside analogs for the treatment of infectious disease and cancer.

In certain embodiments, the disclosure relates to conjugate compounds or salts thereof comprising a sphingolipid or derivative linked by a phosphorus oxide to a nucleotide or nucleoside. In certain embodiments, the phosphorus oxide is a phosphate, phosphonate, polyphosphate, or polyphosphonate, wherein the phosphate, phosphonate or a phosphate in the polyphosphate or polyphosphonate is optionally a phosphorothioate or phosphoramidate. In certain embodiments, the lipid or sphingolipid is covalently bonded to the phosphorus oxide through an amino group or a hydroxyl group.

The nucleotide or nucleoside comprises a heterocycle comprising two or more nitrogen heteroatoms, wherein the substituted heterocycle is optionally substituted with one or more, the same or different alkyl, halogen, or cycloalkyl.

In certain embodiments, the sphingolipid is saturated or unsaturated 2-aminoalkyl or 2-aminooctadecane optionally substituted with one or more substituents. In certain embodiments, the sphingolipid derivative is saturated or unsaturated 2-aminooctadecane-3-ol optionally substituted with one or more substituents. In certain embodiments, the sphingolipid derivative is saturated or unsaturated 2-aminooctadecane-3,5-diol optionally substituted with one or more substituents.

In certain embodiments, the disclosure contemplates pharmaceutical compositions comprising any of the compounds disclosed herein and a pharmaceutically acceptable excipient. In certain embodiments, the pharmaceutical composition is in the form of a pill, capsule, tablet, or saline buffer comprising a saccharide. In certain embodiments, the composition may contain a second active agent such as a pain reliever, anti-inflammatory agent, non-steroidal anti-inflammatory agent, anti-viral agent, anti-biotic, or anti-cancer agent.

In certain embodiments, the disclosure relates to methods of treating or preventing an infection comprising administering an effective amount of a compound of Formulas XXIX-XXXIVb, or a pharmaceutical composition comprising a compound of Formulas XXIX-XXXIVb and a pharmaceutically acceptable excipient, disclosed herein to a subject in need thereof. Typically, the subject is diagnosed with or at risk of an infection from a virus, bacteria, fungi, protozoa, or parasite.

In certain embodiments, the disclosure relates the methods of treating a viral infection comprising administering an effective amount of a compound of Formulas XXIX-XXXIVb, or a pharmaceutical composition comprising a compound of Formulas XXIX-XXXIVb and a pharmaceutically acceptable excipient, disclosed herein to a subject in need thereof. In certain embodiments, the subject is a mammal, for example, a human. In certain embodiments, the subject is diagnosed with a chronic viral infection. In certain embodiments, administration is under conditions such that the viral infection is no longer detected. In certain embodiments, the subject is diagnosed with a RNA virus, DNA virus, or retroviruses. In certain embodiments, the subject is diagnosed with a virus that is a double stranded DNA virus, sense single stranded DNA virus, double stranded RNA virus, sense single stranded RNA virus, antisense single stranded RNA virus, sense single stranded RNA retrovirus or a double stranded DNA retrovirus.

In certain embodiments, the subject is diagnosed with influenza A virus including subtype H1N1, H3N2, H7N9, or H5N1, influenza B virus, influenza C virus, rotavirus A, rotavirus B, rotavirus C, rotavirus D, rotavirus E, human coronavirus, SARS coronavirus, MERS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, coxsackie B virus, Coxsackie virus A1-A16 subtypes (and in certain embodiments, A2-8, A10, A11, A12, A14, and A16 subtypes), norovirus, Rubella virus, lymphocytic choriomeningitis virus (LCMV), chikungunya, Eastern equine encephalitis virus (EEEV), Western equine encephalitis virus (WEEV), Venezuelan equine encephalitis virus (VEEV), Ross River virus, Barmah Forest virus, measles virus, mumps virus, respiratory syncytial virus, rinderpest virus, California encephalitis virus, hantavirus, rabies virus, Ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis D, hepatitis E or human immunodeficiency virus (HIV).

In certain embodiments, the subject is diagnosed with influenza A virus including subtypes H1N1, H3N2, H7N9, H5N1 (low path), and H5N1 (high path) influenza B virus, influenza C virus, rotavirus A, rotavirus B, rotavirus C, rotavirus D, rotavirus E, SARS coronavirus, MERS-CoV, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, coxsackie B virus, norovirus, Rubella virus, lymphocytic choriomeningitis virus (LCMV), measles virus, mumps virus, respiratory syncytial virus, parainfluenza viruses 1 and 3, rinderpest virus, chikungunya, eastern equine encephalitis virus (EEEV), Venezuelan equine encephalitis virus (VEEV), western equine encephalitis virus (WEEV), California encephalitis virus, Rift Valley fever virus (RVFV), heartland virus, La Crosse virus, Marpol virus, Severe fever thrombocytopenia syndrome virus, Pichinde virus, hantavirus, Tacaribe virus, Junin, rabies virus, Ebola virus, Marburg virus, adenovirus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis D, hepatitis E or human immunodeficiency virus (HIV).

As used herein, a “low pathogenicity” virus, e.g., “low pathogenicity influenza” or “low pathogencity H5N1”, refers to a virus strain that results in mild or asymptomatic infections.

As used herein, a “high pathogenicity” virus, e.g., “high pathogenicity influenza” or “high pathogencity H5N1”, refers to a virus strain that results in up to 50% morbidity and mortality, up to 60% morbidity and mortality, up to 70% morbidity and mortality, up to 80% morbidity and mortality, up to 90% morbidity and mortality, or up to 100% morbidity and mortality.

In certain embodiment, the disclosure relates to uses of compounds disclosed herein in the production or manufacture of a medicament for the treatment or prevention of an infectious disease, viral infection, or cancer.

In certain embodiments, the disclosure relates to derivatives of compounds disclosed herein or any of the formula.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In certain embodiments, a pharmaceutical agent, which may be in the form of a salt or prodrug, is administered in methods disclosed herein that is specified by a weight. This refers to the weight of the recited compound. If in the form of a salt or prodrug, then the weight is the molar equivalent of the corresponding salt or prodrug.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

### Definitions

Prior to describing the various embodiments, the following definitions are provided and should be used unless otherwise indicated.

As used herein, the term “deuterium” or “D” refers to the isotopic abundance of D relative to H (hydrogen) is at least 50%, at least 75%, or at least 90%.

As used herein, the term “phosphorus oxide” refers to any variety of chemical moieties that contain a phosphorus-oxygen (P—O or P═O) bond. When used as linking groups herein, the joined molecules may bond to oxygen or directly to the phosphorus atoms. The term is intended to include, but are not limited to phosphates, in which the phosphorus is typically bonded to four oxygens and phosphonates, in which the phosphorus is typically bonded to one carbon and three oxygens. A “polyphosphate” generally refers to phosphates linked together by at least one phosphorus-oxygen-phosphorus (P—O—P) bond. A “polyphosphonate” refers to a polyphosphate that contains at least one phosphorus-carbon (C—P—O—P) bond. In addition to containing phosphorus-oxygen bond, phosphorus oxides may contain a phosphorus-thiol (P—S or P═S) bond and/or a phosphorus-amine (P—N) bond, respectively referred to as phosphorothioate or phosphoramidate. In phosphorus oxides, the oxygen atom may form a double or single bond to the phosphorus or combinations, and the oxygen may further bond with other atoms such as carbon or may exist as an anion which is counter balanced with a cation, e.g., metal or quaternary amine.

The term “subject” (alternatively “patient” or “participant”, as in a clinical trial participant) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), Lagomorpha (rabbits), rodents (e.g., rats or mice), Procyon lotor (e.g., raccoons). In particular embodiments, the subject is human.

The term “subject in need thereof” (alternatively “patient in need thereof”) as used herein refers to a subject diagnosed with, or suspected of having, a viral infection, such as infection by SARS-CoV-2 (either symptomatic or asymptomatic); a subject at risk of being exposed to a viral infection, such as at risk of being exposed to a viral infection, such as infection by SARS-CoV-2 (such as, for example, health care workers who may be at risk of exposure to SARS-CoV-2); a subject exposed to a viral infection, such as infection by SARS-CoV-2 (such as household contacts of COVID-19 patients or asymptomatic patients infected with SARS-CoV-2), as defined herein.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent can be administered prior to, together with, or after the additional treatment, or a combination thereof.

As used herein, “alkyl” means a straight or branched chain saturated hydrocarbon moieties such as those containing from 1 to 24 carbon atoms. A “higher alkyl” refers to saturated hydrocarbon having 24 or more carbon atoms. A “C₆-C₁₆” refers to an alkyl containing 6 to 16 carbon atoms. Likewise a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. A “lower alkyl” group is an alkyl group containing from one to six (e.g., from one to four) carbon atoms. The term alkyl group can also be a C₁ alkyl, C₁-C₂ alkyl, C₁-C₃ alkyl, C₁-C₄ alkyl, C₁-C₅ alkyl, C₁-C₆ alkyl, C₁-C₇ alkyl, C₁-C₈ alkyl, C₁-C₉ alkyl, C₁-C₁₀ alkyl, and the like up to and including a C₁-C₂₄ alkyl.

As used herein, the term “alkenyl” refers to unsaturated, straight or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C.

As used herein, the term “alkynyl” represents straight or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which can be saturated or unsaturated (but not aromatic), monocyclic or polycyclic, and wherein the nitrogen and sulfur heteroatoms can be optionally oxidized, and the nitrogen heteroatom can be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and, the like, where optionally one or more pair of substituents together with the atoms to which they are bonded form a 3 to 7 member ring.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems can, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems can be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bride (i.e., —(C═O)alkyl).

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., —NHS(═O)₂aryl).

“Alkylsulfinyl” refers to an alkyl as defined above with the indicated number of carbon atoms attached through a sulfinyl bridge (i.e., —S(═O)alkyl).

The terms “cycloalkyl” and “cycloalkenyl” refer to mono-, bi-, or tri homocyclic ring groups of 3 to 15 carbon atoms which are, respectively, fully saturated and partially unsaturated.

The term “cycloalkenyl” includes bi- and tricyclic ring systems that are not aromatic as a whole, but contain aromatic portions (e.g., fluorene, tetrahydronapthalene, dihydroindene, and the like). The rings of multi-ring cycloalkyl groups can be either fused, bridged and/or joined through one or more spiro unions. The terms “substituted cycloalkyl” and “substituted cycloalkenyl” refer, respectively, to cycloalkyl and cycloalkenyl groups substituted with one or more groups, preferably selected from aryl, substituted aryl, heterocyclo, substituted heterocyclo, carbocyclo, substituted carbocyclo, halo, hydroxy, alkoxy (optionally substituted), aryloxy (optionally substituted), alkylester (optionally substituted), arylester (optionally substituted), alkanoyl (optionally substituted), aryol (optionally substituted), cyano, nitro, amino, substituted amino, amido, lactam, urea, urethane, sulfonyl, and the like.

The terms “halogen” and “halo” refer to fluorine, chlorine, bromine, and iodine.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

As used herein, the designation of a polyvalent moiety without specifying the specific order of attachment is intended to cover all possible arrangements. By way of example, a compound represented by the formula:

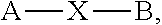

wherein X is NHC(═O) embraces both:

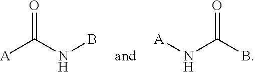

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃. A group having the subscript ‘0’ is understood to represent a null group as well. By way of example, in the compound CH₃—(X)_{z}—CH₃, if X is CH₂ and z is 0, then the compound has the formula CH₃—CH₃.

In certain instances, two or more variable groups may together form a ring. It is understood that any depicted atoms separated the identified groups will themselves form part of the ring:

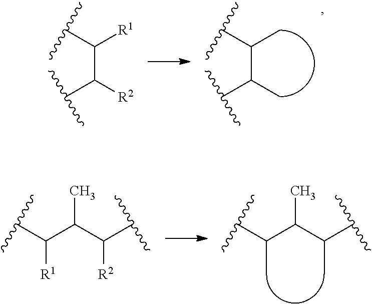

When the variable groups are substituted on an aromatic system the new ring will be a fused ring, and unless specified to the contrary may be either aromatic or non-aromatic, carbocyclic or heterocyclic:

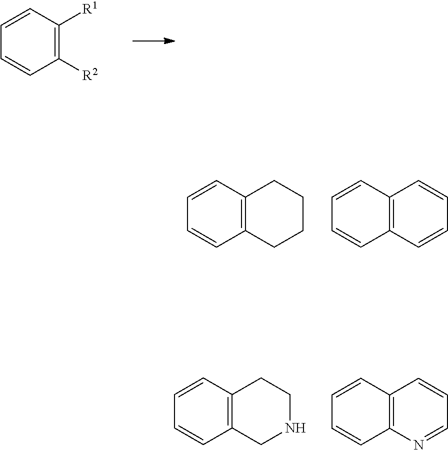

The ring may further be defined by the number of carbon atoms in the specific ring formed by the variable groups, which includes the atoms separating the variable groups:

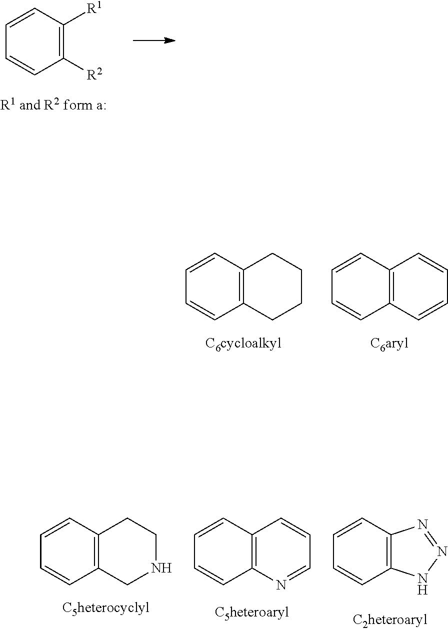

With the exception of the C₂heteroaryl above, each of the above is results with R¹ and R² together form a six membered (or six atom) ring.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

Examples of prodrugs that can be used to improve bioavailability include esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, sulfenyl, optionally substituted sulfenyl, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally susbstituted hemiaminal, acetal, optionally substituted acetal, hemiacetal, optionally susbstituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, bis-(acyloxybenzyl)esters, optionally substituted bis-(acyloxybenzyl)esters, (acyloxybenzyl)esters, optionally substituted (acyloxybenzyl)esters, and BAB-esters. As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative.

The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

Compounds described herein can contain one or more double bonds and, thus, potentially give rise to cis/trans (E/Z) isomers, as well as other conformational isomers. Unless stated to the contrary, the disclosure includes all such possible isomers, as well as mixtures of such isomers.

As used herein, the following three structural formulas depict the same compound:

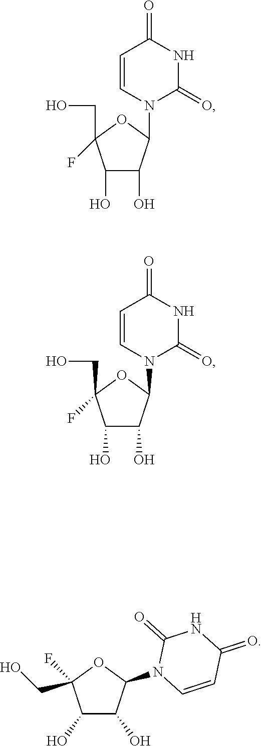 [SMILES: O=c1ccn(C2OC(F)(CO)C(O)C2O)c(=O)[nH]1 ; O=c1ccn([C@@H]2O[C@](F)(CO)[C@@H](O)[C@H]2O)c(=O)[nH]1 ; O=c1ccn([C@@H]2O[C@](F)(CO)[C@@H](O)[C@H]2O)c(=O)[nH]1]

This convention is followed for each of the nucleoside derivatives disclosed herein.

With the exception of the nucleoside convention exemplified above, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. It is understood that use of a wedge or hash is only one representation of a stereochemical descriptor. All stereoisomers, including enantiomers and diastereomers, as well as their racemic and optically pure forms and mixtures thereof in any ratio, are included within Formulas XXIX to XXXIb and are provided by the present disclosure.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

It is understood that enantiomeric and/or diastereomeric forms exist of a given structure, and that flat bonds indicate that all stereoisomeric forms of the depicted structure may be present. Moreover, where enantiomeric and/or diastereomeric forms exist of a given structure, flat bonds and the presence of a “*” symbol indicate that the composition is made up of at least 60%, at least 70%, at least 80%, or at least 90%, by weight, of a single isomer with unknown stereochemistry. It is further understood that where enantiomeric and/or diastereomeric forms exist of a given structure, wedged or hashed bonds indicate the composition is made up of at least 60%, at least 70%, at least 80%, or at least 90%, by weight, of a single enantiomer or diastereomer with known stereochemistry. As appropriate, combinations of the above notation may be used. Exemplified species may contain stereogenic centers with known stereochemistry and stereogenic centers with unknown stereochemistry, stereochemistry.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this disclosure. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non-isotopically labeled reagent.

The compounds described in the disclosure can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the disclosure to form solvates and hydrates. Unless stated to the contrary, the disclosure includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

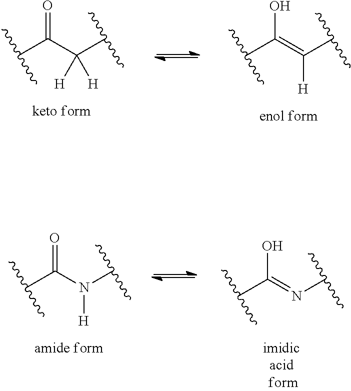 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the disclosure includes all such possible tautomers.

It is known that chemical substances form solids which are present in different states of order which are termed polymorphic forms or modifications. The different modifications of a polymorphic substance can differ greatly in their physical properties. The compounds according to the disclosure can be present in different polymorphic forms, with it being possible for particular modifications to be metastable. Unless stated to the contrary, the disclosure includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

### Compounds

In certain embodiments, the disclosure relates to nucleosides conjugated to a phosphorus moiety and pharmaceutically acceptable salts thereof.

In certain embodiments, the disclosure relates to a compound of Formula XXIX,

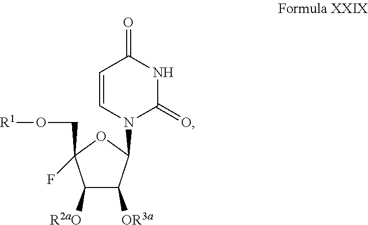

or a pharmaceutically acceptable salt thereof,
wherein R¹ is selected from a group having a structure represented by a formula:

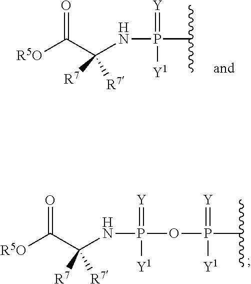

wherein Y is O or S;
wherein Y¹ is OY³ or BH₃⁻M⁺; preferably OY³;
wherein Y³ is hydrogen, aryl, heteroaryl, or heterocyclyl, wherein Y³ is optionally substituted with one or more, the same or different, R¹⁰; preferably Y³ is C₆-C₁₂aryl or C₁-C₁₂heteroaryl;
wherein each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, or (C═O)OC₁-C₆ alkyl, wherein said alkyl groups are optionally independently substituted with one or more, the same or different, R¹⁰;
wherein R⁴⁰ is in each case independently selected from hydrogen and C₁-C₆alkyl, optionally substituted one more times by R¹⁰;
wherein R^{2a} and R^{3a} can together form a 5-7 membered heterocyclic ring, for example R^{2a} and R^{3a} can together C(CH₃)₂, C(═O) or C(═S);
wherein R⁵ is hydrogen, alkyl, carbocyclyl, heterocarbocyclyl, aryl, heteroaryl, heterocyclyl, cycloalkyl, cycloalkenyl, or lipid; and wherein R⁵ is optionally substituted with one or more, the same or different, R¹⁰; preferably R⁵ is hydrogen, C₁-C₈alkyl, C₆-C₁₂aryl, C₁-C₁₂heteroaryl, C₂-C₈heterocyclyl, C₃-C₈cycloalkyl, or C₅-C₈cycloalkenyl, and wherein R⁵ is optionally substituted with one or more, the same or different, R¹⁰;
wherein R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ and R^{7′} are optionally substituted with one or more, the same or different, R¹⁰; preferably R⁷ and R^{7′} are independently hydrogen, C₁-C₈alkyl, C₂-C₈alkenyl, C₂-C₈alkynyl, C₆-C₁₂aryl, C₁-C₁₂heteroaryl, C₂-C₈heterocyclyl, C₃-C₈cycloalkyl, or C₅-C₈cycloalkenyl, and wherein R⁵ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, polyethylene glycol, nitro, or carbonyl; and
wherein lipid is independently a C₁₁-C₂₂alkyl, C₁₁-C₂₂alkoxy, or aryl substituted with an C₆-C₁₈ alkyl group.

In certain embodiments, one of R⁷ and R^{7′} are hydrogen, and the other is not. In certain embodiments, R⁷ is hydrogen and R^{7′} is not hydrogen. In some embodiments, R⁷ is not hydrogen and R^{7′} is hydrogen.

In certain embodiments, the disclosure relates to a compound of Formula XXIX,

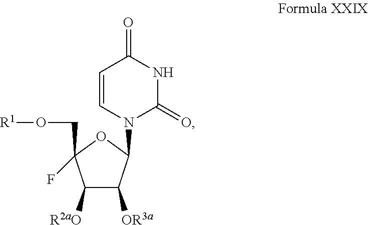

or a pharmaceutically acceptable salt thereof,
wherein R¹ is selected from a group having a structure represented by a formula:

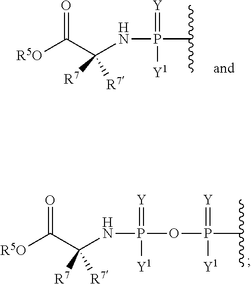

wherein Y is O or S;
wherein Y¹ is OY³ or BH₃⁺M⁺;
wherein Y³ is hydrogen, aryl, heteroaryl, or heterocyclyl, wherein Y³ is optionally substituted with one or more, the same or different, R¹⁰;
wherein R^{2a} and R^{3a} are each hydrogen;
wherein R⁵ is hydrogen, alkyl, carbocyclyl, heterocarbocyclyl, aryl, heteroaryl, heterocyclyl, cycloalkyl, cycloalkenyl, or lipid; and wherein R⁵ is optionally substituted with one or more, the same or different, R¹⁰;
wherein R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
wherein lipid is independently a C₁₁-C₂₂alkyl, C₁₁-C₂₂alkoxy, or aryl substituted with an C₆-C₁₈ alkyl group.

In certain embodiments, one of R⁷ and R^{7′} are hydrogen, and the other is not. In certain embodiments, R⁷ is hydrogen and R^{7′} is not hydrogen. In some embodiments, R⁷ is not hydrogen and R^{7′} is hydrogen.

In exemplified embodiments of Formula XXIX, the disclosure relates to a compound of Formula XXIXa,

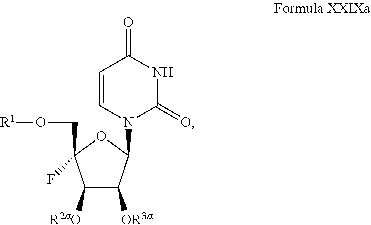

or a pharmaceutically acceptable salt thereof,
wherein R¹ is selected from a group having a structure represented by a formula:

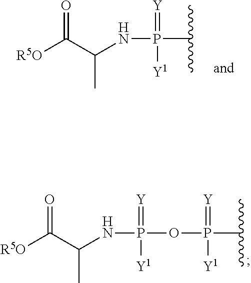

wherein Y is O or S;
wherein Y¹ is OY³ or BH₃⁻M⁺;
wherein Y³ is hydrogen, aryl, heteroaryl, or heterocyclyl; and wherein Y³ is optionally substituted with one or more, the same or different, R¹⁰;
wherein each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, polyethylene glycol, aryl, and lipid;
wherein R^{2a} and R^{3a} can each be optionally independently substituted with one or more, the same or different, R¹⁰; and
wherein R^{2a} and R^{3a} can together form a 5-7 membered heterocyclic ring, for example R^{2a} and R^{3a} can together C(CH₃)₂, C(═O) or C(═S);
wherein R⁵ is hydrogen, alkyl, carbocyclyl, heterocarbocyclyl, aryl, heteroaryl, heterocyclyl, cycloalkyl, cycloalkenyl, polyethylene glycol, aryl substituted with an alkyl group, lipid; and wherein R⁵ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
wherein lipid is independently a C₁₁-C₂₂alkyl, C₁₁-C₂₂alkoxy, or aryl substituted with an C₆-C₁₈ alkyl group.

In exemplified embodiments of Formula XXIX, the disclosure relates to a compound of Formula XXIXa,

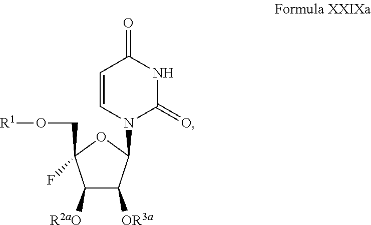

or a pharmaceutically acceptable salt thereof,
wherein R¹ is selected from a group having a structure represented by a formula:

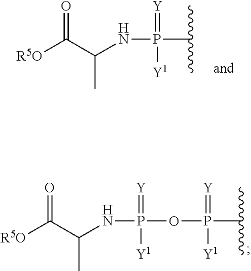

wherein Y is O or S;
wherein Y¹ is OY³ or BH₃⁻M⁺;
wherein Y³ is hydrogen, aryl, heteroaryl, or heterocyclyl; and wherein Y³ is optionally substituted with one or more, the same or different, R¹⁰;
(C═O)C₁-C₆ alkyl, (C═O)NR⁴⁰C₁-C₆ alkyl, (C═O)N(C₁-C₆ alkyl)₂, or (C═O)OC₁-C₆ alkyl, wherein said alkyl groups are optionally independently substituted with one or more, the same or different, R¹⁰;
wherein R⁴⁰ is in each case independently selected from hydrogen and C₁-C₆alkyl, optionally substituted one more times by R¹⁰;
wherein R⁵ is hydrogen, alkyl, carbocyclyl, heterocarbocyclyl, aryl, heteroaryl, heterocyclyl, cycloalkyl, cycloalkenyl, or lipid; and wherein R⁵ is optionally substituted with one or more, the same or different, R¹⁰;
wherein R⁷ and R^{7′} are each independently selected from hydrogen, deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl, wherein R⁷ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
wherein lipid is independently a C₁₁-C₂₂alkyl, C₁₁-C₂₂alkoxy, or aryl substituted with an C₆-C₁₈ alkyl group.

In exemplified embodiments of Formula XXIX, the disclosure relates to a compound of Formula XXIXb,

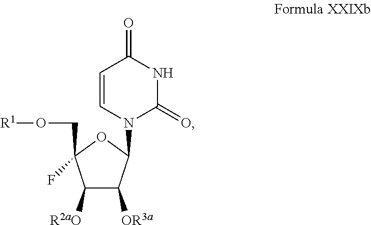

or a pharmaceutically acceptable salt thereof,
wherein R¹ is selected from a group having a structure represented by a formula:

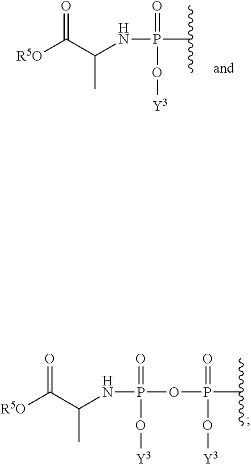

wherein Y³ is hydrogen, aryl, heteroaryl, or heterocyclyl; and wherein Y³ is optionally substituted with one or more, the same or different, R¹⁰;
wherein each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, polyethylene glycol, aryl, and lipid;
wherein R^{2a} and R^{3a} can each be optionally independently substituted with one or more, the same or different, R¹⁰; and
wherein R^{2a} and R^{3a} can together form a 5-7 membered heterocyclic ring, for example R^{2a} and R^{3a} can together C(CH₃)₂, C(═O) or C(═S);
wherein R⁵ is hydrogen, alkyl, carbocyclyl, heterocarbocyclyl, aryl, heteroaryl, heterocyclyl, cycloalkyl, cycloalkenyl, polyethylene glycol, aryl substituted with an alkyl group, lipid; and wherein R⁵ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
wherein lipid is independently a C₁₁-C₂₂alkyl, C₁₁-C₂₂alkoxy, or aryl substituted with an C₆-C₁₈ alkyl group.

In exemplified embodiments of Formula XXIX, the disclosure relates to a compound of Formula XXIXb,

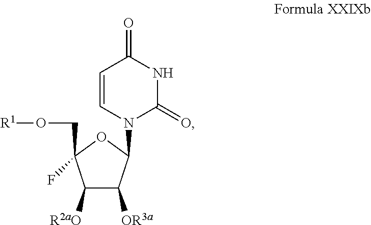

or a pharmaceutical or physiological salt thereof,
wherein R¹ is selected from a group having a structure represented by a formula:

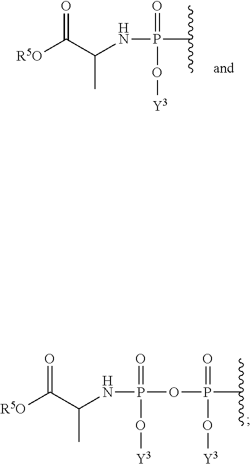

wherein Y³ is hydrogen, aryl, heteroaryl, or heterocyclyl; and wherein Y³ is optionally substituted with one or more, the same or different, R¹⁰;
wherein R⁵ is hydrogen, alkyl, carbocyclyl, heterocarbocyclyl, aryl, heteroaryl, heterocyclyl, cycloalkyl, cycloalkenyl, or lipid; and wherein R⁵ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
wherein lipid is independently a C₁₁-C₂₂alkyl, C₁₁-C₂₂alkoxy, or aryl substituted with an C₆-C₁₈ alkyl group.

In exemplified embodiments of Formula XXIX, the disclosure relates to a compound of Formula XXIXc,

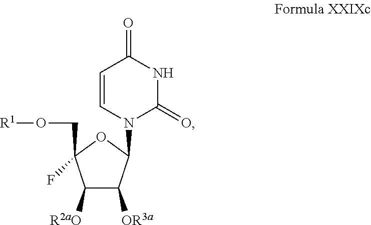

or a pharmaceutically acceptable salt thereof,
wherein R¹ is:

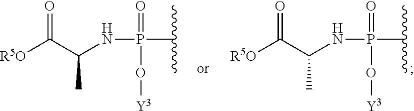

preferably R¹ is:

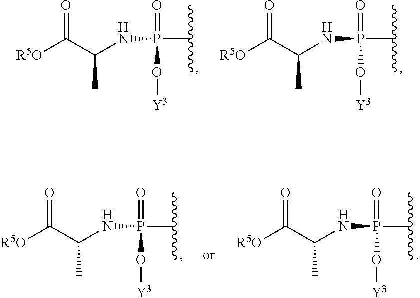

In some embodiments, the R¹ moiety has a stereochemical purity of at least 75 mol %, at least 85 mol %, at least 90 mol %, at least 95 mol %, or at least 99 mol % the depicted stereoisomer at the phosphorous atom. The skilled person understands the above moiety can be derived from alanine, and therefore is essentially stereochemically pure at the methyl bearing carbon.

In certain implementations Y³ is hydrogen, aryl, heteroaryl, or heterocyclyl; and wherein Y³ is optionally substituted with one or more, the same or different, R¹⁰;
wherein R⁵ is hydrogen, alkyl, carbocyclyl, heterocarbocyclyl, aryl, heteroaryl, heterocyclyl, cycloalkyl, cycloalkenyl, polyethylene glycol, aryl substituted with an alkyl group, lipid; and wherein R⁵ is optionally substituted with one or more, the same or different, R¹⁰;
R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
   wherein lipid is independently a C₁₁-C₂₂alkyl, C₁₁-C₂₂alkoxy, or aryl substituted with an C₆-C₁₈ alkyl group.

In exemplified embodiments of Formula XXIX, the disclosure relates to a compound of Formula XXIXc,

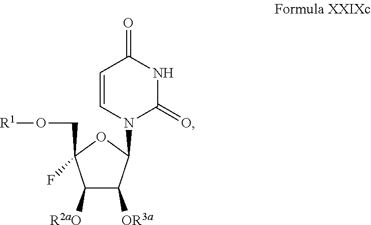

or a pharmaceutical or physiological salt thereof,
wherein R¹ is:

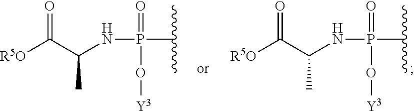

preferably R¹ is:

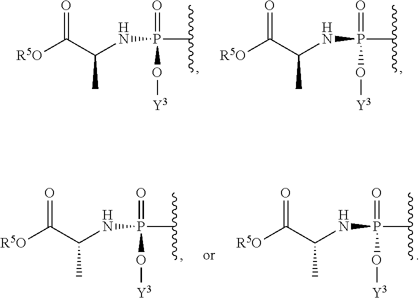

In some embodiments, the R¹ moiety has a stereochemical purity of at least 75 mol %, at least 85 mol %, at least 90 mol %, at least 95 mol %, or at least 99 mol % the depicted stereoisomer at the phosphorous atom. The skilled person understand the above moiety can be derived from alanine, and therefore is essentially stereochemically pure at the methyl bearing carbon.

In some implementations Y³ is hydrogen, aryl, heteroaryl, or heterocyclyl; and wherein Y³ is optionally substituted with one or more, the same or different, R¹⁰;
wherein R⁵ is hydrogen, alkyl, carbocyclyl, heterocarbocyclyl, aryl, heteroaryl, heterocyclyl, cycloalkyl, cycloalkenyl, or lipid; and wherein R⁵ is optionally substituted with one or more, the same or different, R¹⁰;
wherein R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
   wherein lipid is independently a C₁₁-C₂₂alkyl, C₁₁-C₂₂alkoxy, or aryl substituted with an C₆-C₁₈ alkyl group.

In exemplified embodiments of Formula XXIX-Formula XXIXc, R¹ is a group having a structure represented by a formula:

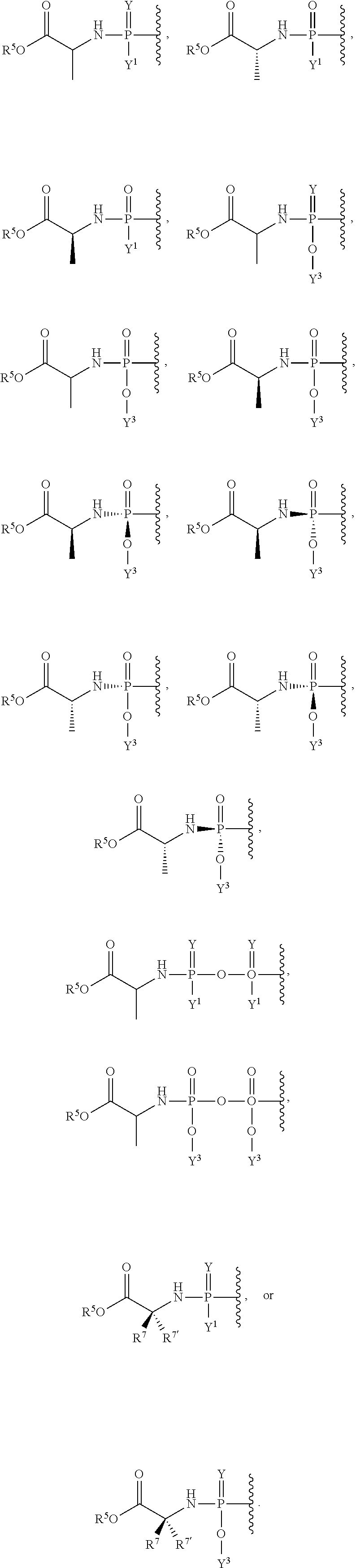

In exemplified embodiments of Formula XXIX-Formula XXIXc, R¹ is a group having a structure represented by a formula:

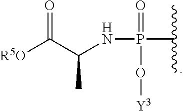

In exemplified embodiments of Formula XXIX-Formula XXIXc, R¹ is a group having a structure represented by a formula:

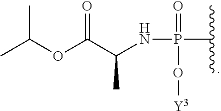

In exemplified embodiments of Formula XXIX-Formula XXIXc, R¹ is a group having a structure represented by a formula:

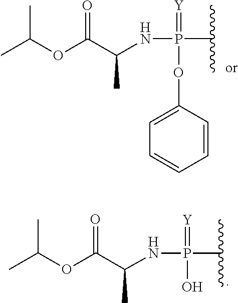

In exemplified embodiments of Formula XXIX-Formula XXIXc, R¹ is a group as disclosed herein with the proviso that R¹ is not:

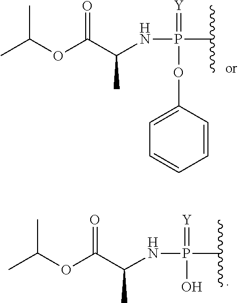

In exemplified embodiments of Formula XXIX-Formula XXIXc, R¹ is a group having a structure represented by a formula:

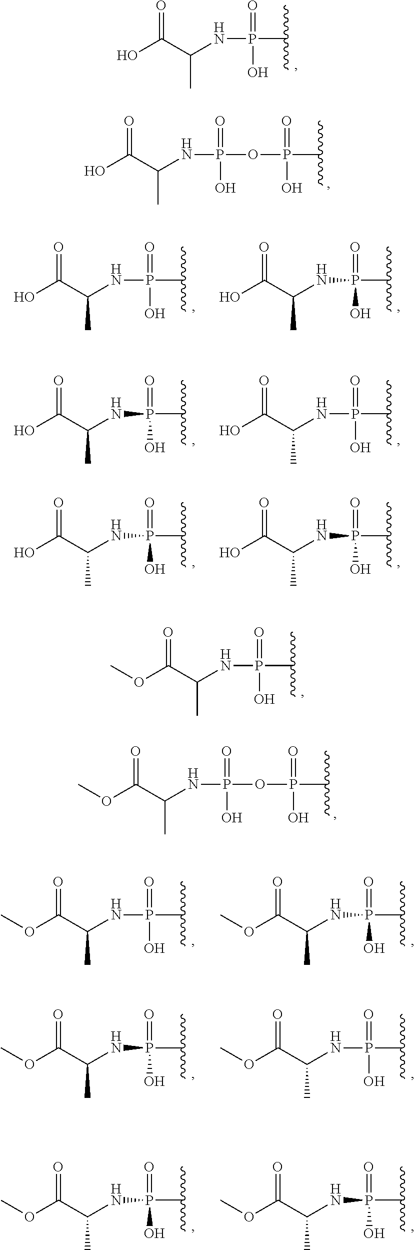

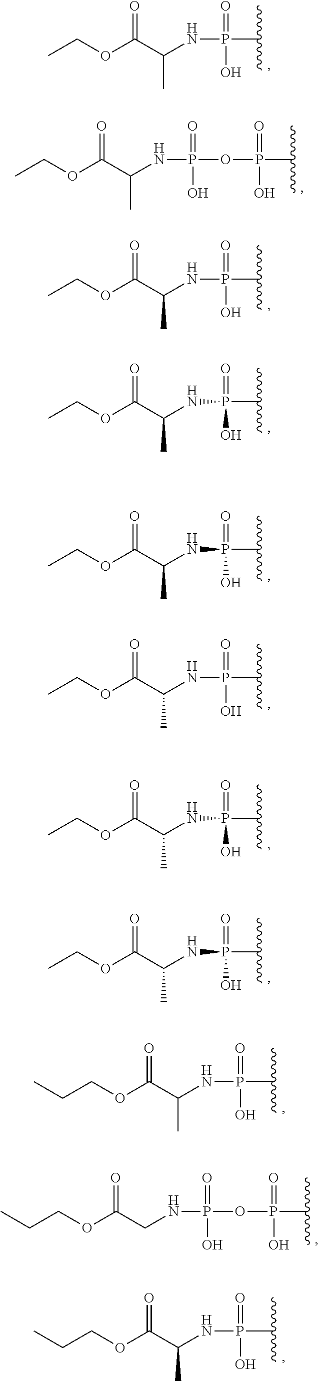

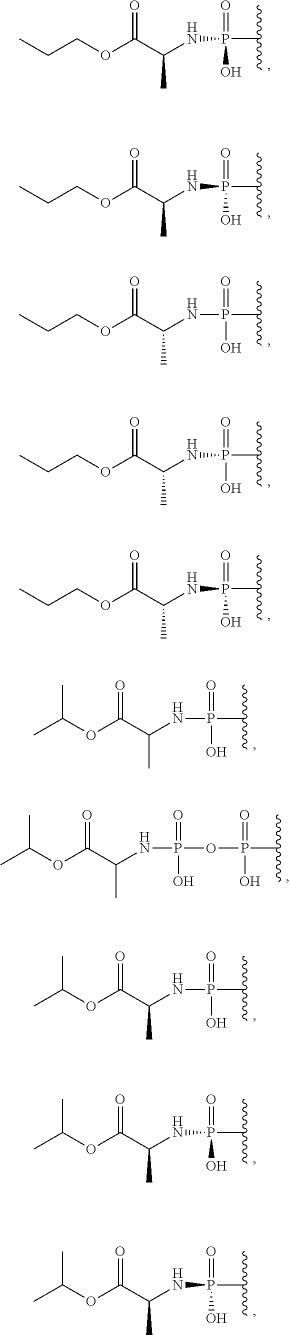

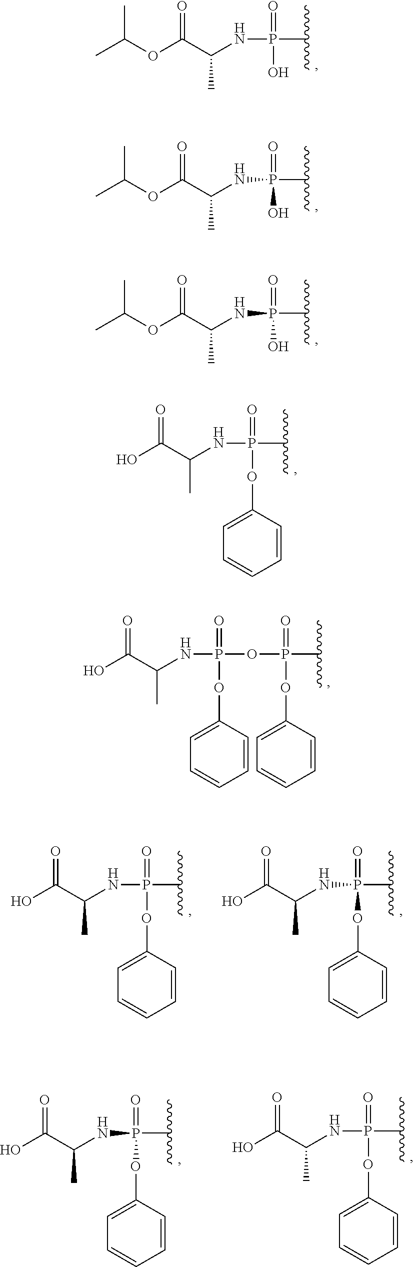

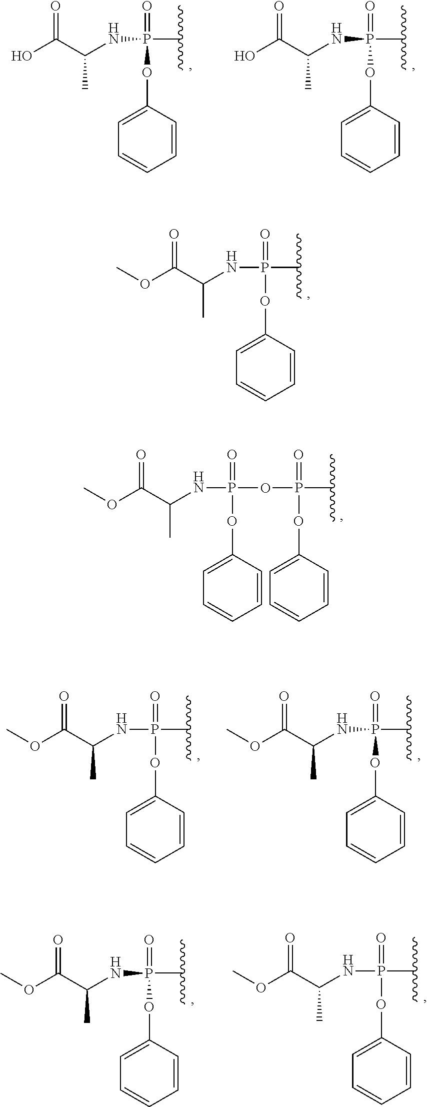

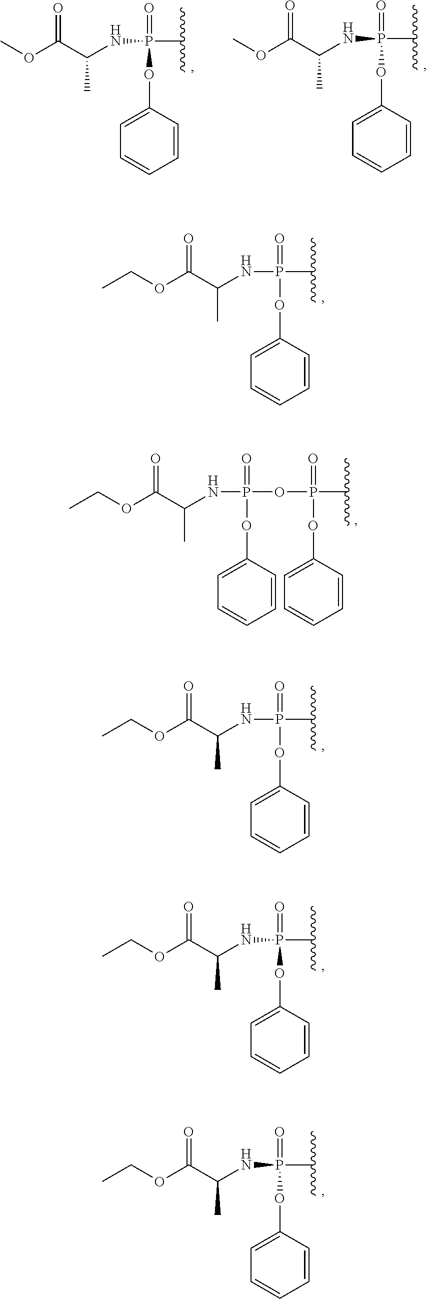

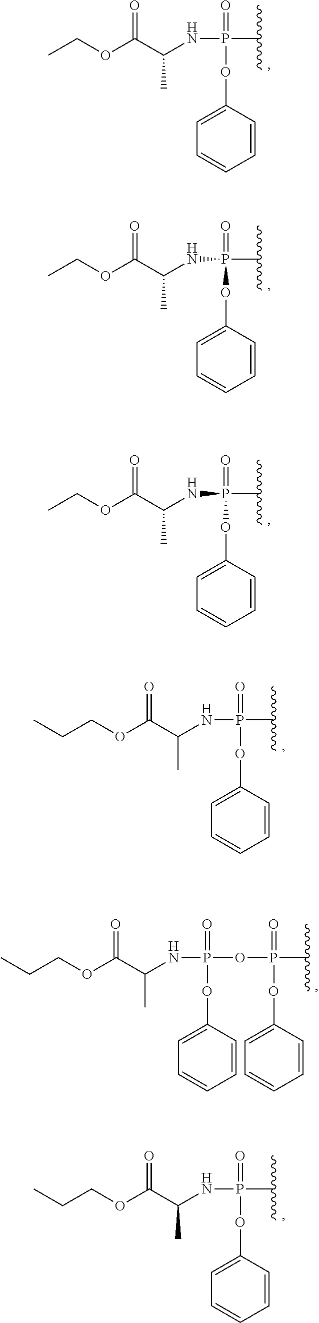 [SMILES: *P(=O)(N[C@H](C)C(=O)OCC)Oc1ccccc1 ; *[P@@](=O)(N[C@H](C)C(=O)OCC)Oc1ccccc1 ; *[P@](=O)(N[C@H](C)C(=O)OCC)Oc1ccccc1 ; *P(=O)(NC(C)C(=O)OCCC)Oc1ccccc1 ; *P(=O)(Oc1ccccc1)OP(=O)(NC(C)C(=O)OCCC)Oc1ccccc1 ; *P(=O)(N[C@@H](C)C(=O)OCCC)Oc1ccccc1]

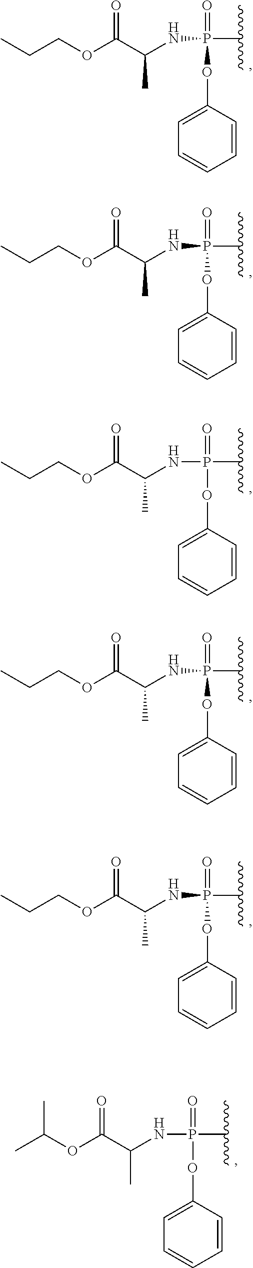

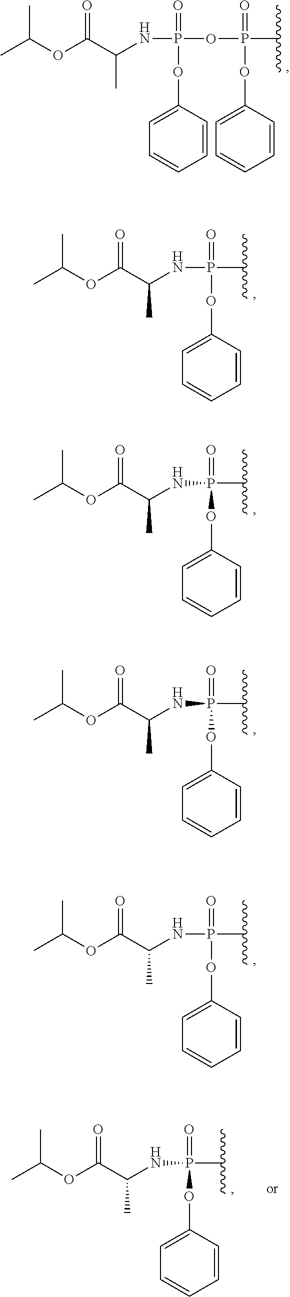 [SMILES: *P(=O)(Oc1ccccc1)OP(=O)(NC(C)C(=O)OC(C)C)Oc1ccccc1 ; *P(=O)(N[C@@H](C)C(=O)OC(C)C)Oc1ccccc1 ; *[P@@](=O)(N[C@@H](C)C(=O)OC(C)C)Oc1ccccc1 ; *[P@](=O)(N[C@@H](C)C(=O)OC(C)C)Oc1ccccc1 ; *P(=O)(N[C@H](C)C(=O)OC(C)C)Oc1ccccc1 ; *[P@@](=O)(N[C@H](C)C(=O)OC(C)C)Oc1ccccc1]

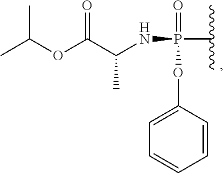 [SMILES: *[P@](=O)(N[C@H](C)C(=O)OC(C)C)Oc1ccccc1]

wherein the stereochemical purity at the phosphorous atom is at least 75 mol %, at least 85 mol %, at least 90 mol %, at least 95 mol %, or at least 99 mol % of the depicted stereoisomer.

In exemplified embodiments of Formula XXIX-Formula XXIXc, R¹ is a group having a structure represented by a formula:

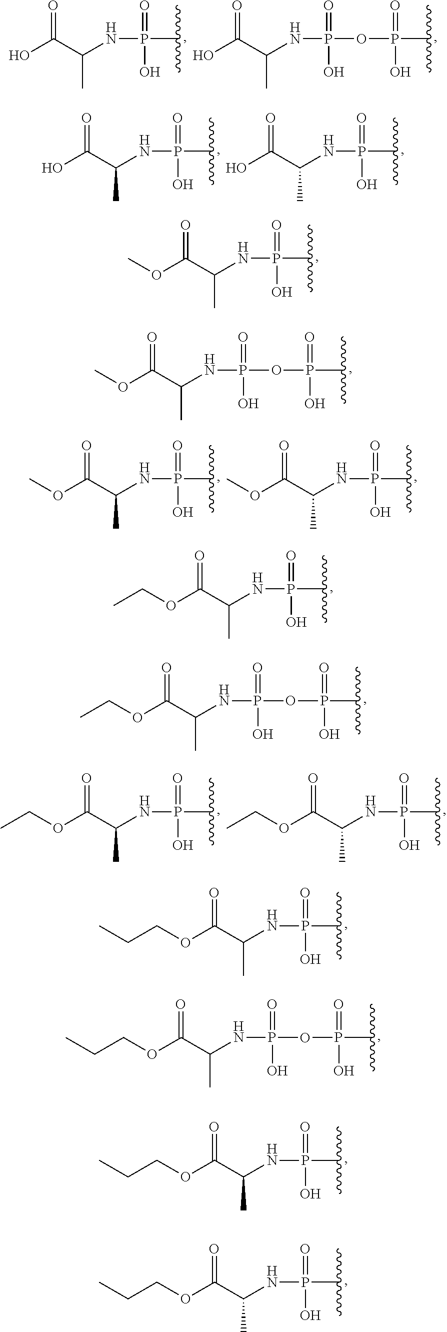

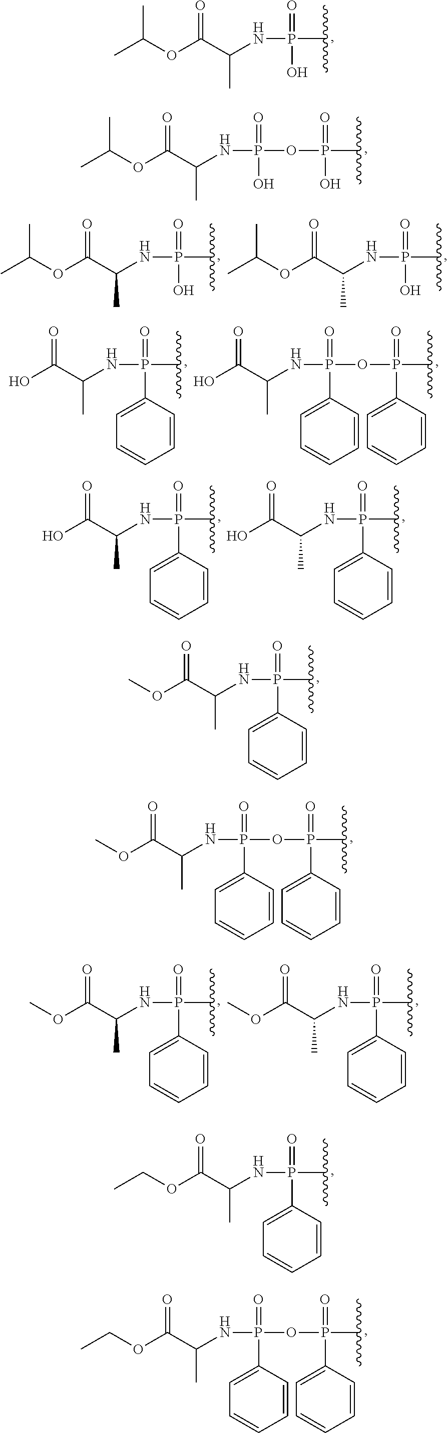

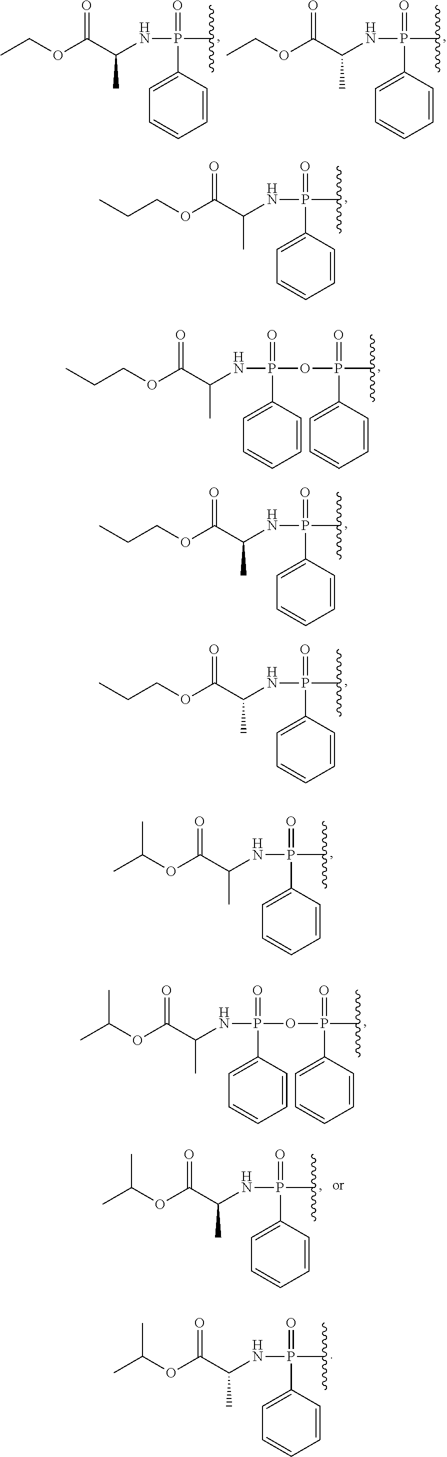

In exemplified embodiments of Formula XXIX-Formula XXIXc, R¹ is a group having a structure represented by a formula:

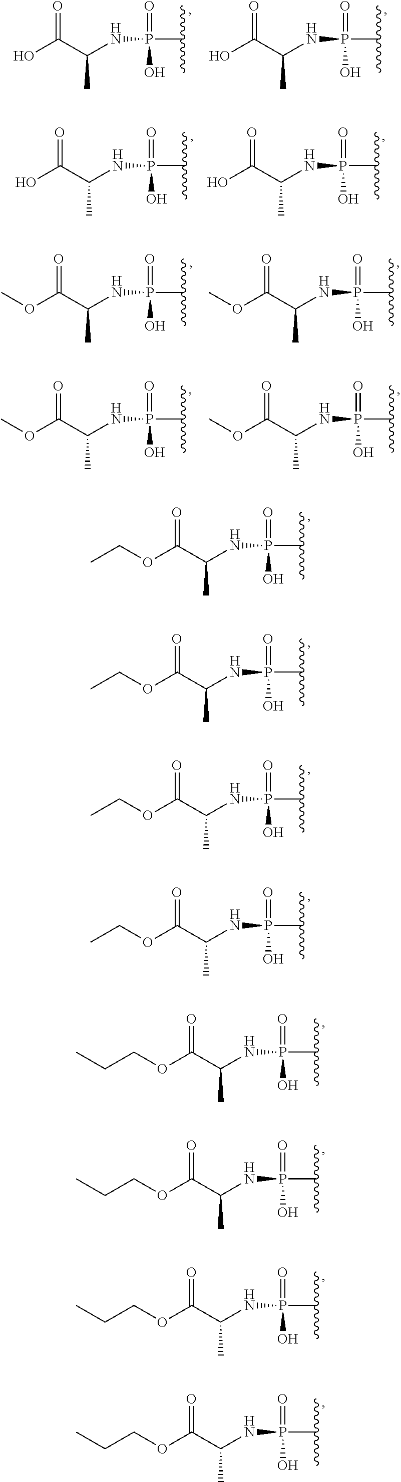

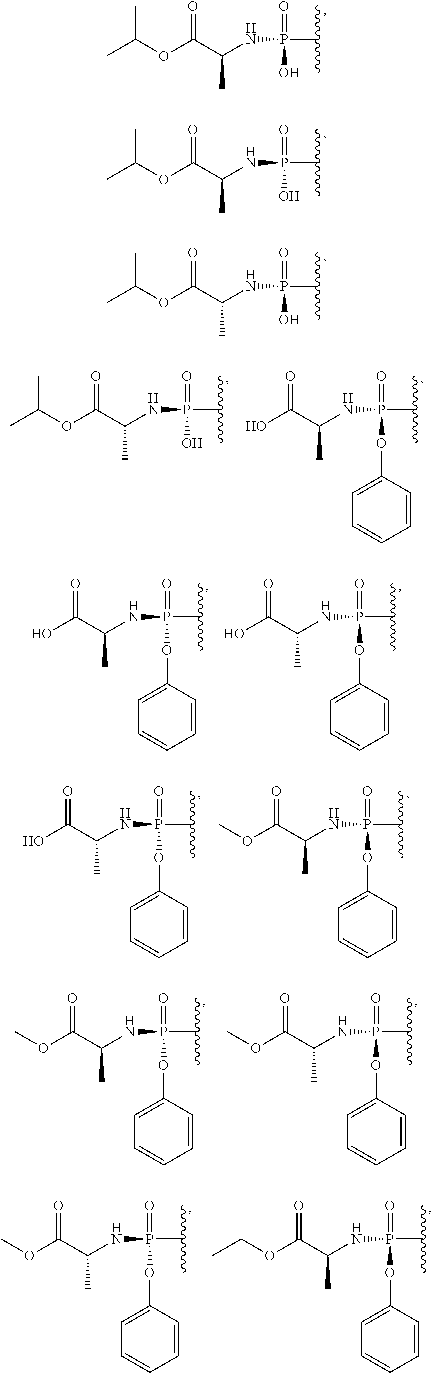

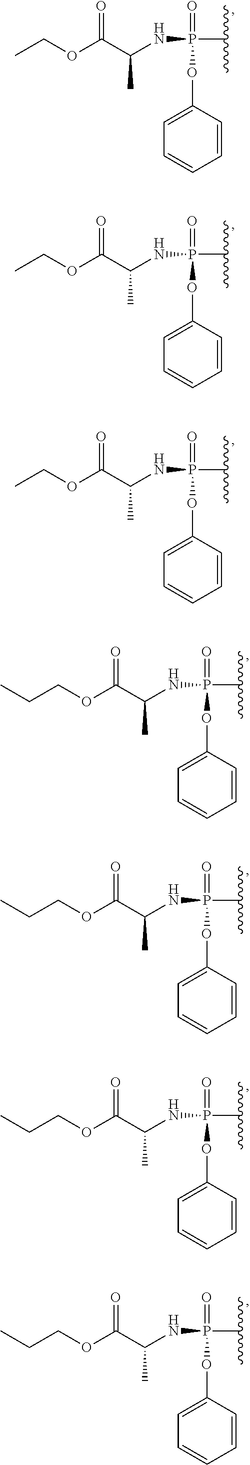

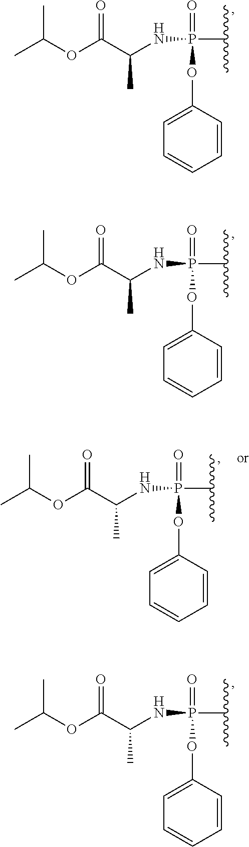 [SMILES: *[P@@](=O)(N[C@@H](C)C(=O)OC(C)C)Oc1ccccc1 ; *[P@](=O)(N[C@@H](C)C(=O)OC(C)C)Oc1ccccc1 ; *[P@@](=O)(N[C@H](C)C(=O)OC(C)C)Oc1ccccc1 ; *[P@](=O)(N[C@H](C)C(=O)OC(C)C)Oc1ccccc1]

wherein the stereochemical purity at the phosphorous atom is at least 75 mol %, at least 85 mol %, at least 90 mol %, at least 95 mol %, or at least 99 mol % of the depicted stereoisomer.

In exemplified embodiments of Formula XXIX-Formula XXIXc, R¹ is a group having a structure represented by a formula:

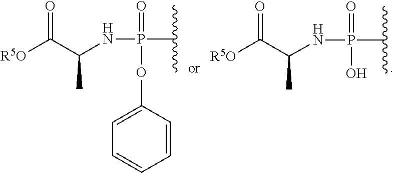

In exemplified embodiments of Formula XXIX-Formula XXIXc, R⁵ is selected from lipid, methyl, ethyl, propyl, isopropyl, butyl, i-butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIX-Formula XXIXc, R⁵ is selected from lipid, ethyl, propyl, isopropyl, butyl, i-butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIX-Formula XXIXc, R⁵ is selected from lipid, propyl, isopropyl, butyl, i-butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIX-Formula XXIXc, R⁵ is selected from lipid, butyl, i-butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIX-Formula XXIXc, R⁵ is selected from lipid, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.

In exemplified embodiments of Formula XXIX-Formula XXIXc, R⁵ is selected from methyl, ethyl, propyl, isopropyl, butyl, i-butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl, and 2,6-dimethylphenyl.

In exemplified embodiments of Formula XXIX-Formula XXIXc, R⁵ is selected from ethyl, propyl, butyl, i-butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, and cyclohexyl.

In exemplified embodiments of Formula XXIX-Formula XXIXc, R⁵ is selected from and phenyl 2,6-dimethylphenyl.

In exemplified embodiments of Formula XXIX-Formula XXIXc, R⁵ is selected from cyclopropyl, cyclobutyl, cyclopentyl, and cyclohexyl.

In exemplified embodiments of Formula XXIX-Formula XXIXc, R⁵ is selected from pentyl, s-pentyl, t-pentyl, neopentyl, 3-pentyl, hexyl, t-hexyl, and 4-septyl.

In exemplified embodiments of Formula XXIX-Formula XXIXc, the compound is selected from:

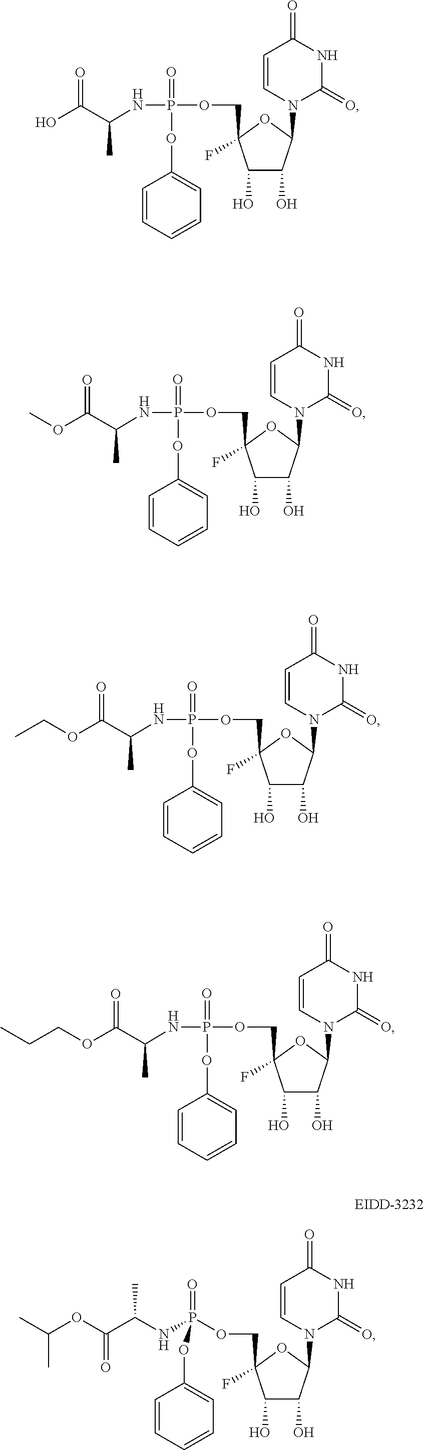 [SMILES: C[C@H](NP(=O)(OC[C@@]1(F)O[C@@H](n2ccc(=O)[nH]c2=O)[C@H](O)[C@@H]1O)Oc1ccccc1)C(=O)O ; COC(=O)[C@H](C)NP(=O)(OC[C@@]1(F)O[C@@H](n2ccc(=O)[nH]c2=O)[C@H](O)[C@@H]1O)Oc1ccccc1 ; CCOC(=O)[C@H](C)NP(=O)(OC[C@@]1(F)O[C@@H](n2ccc(=O)[nH]c2=O)[C@H](O)[C@@H]1O)Oc1ccccc1 ; CCCOC(=O)[C@H](C)NP(=O)(OC[C@@]1(F)O[C@@H](n2ccc(=O)[nH]c2=O)[C@H](O)[C@@H]1O)Oc1ccccc1 ; CC(C)OC(=O)[C@H](C)N[P@](=O)(OC[C@@]1(F)O[C@@H](n2ccc(=O)[nH]c2=O)[C@H](O)[C@@H]1O)Oc1ccccc1]

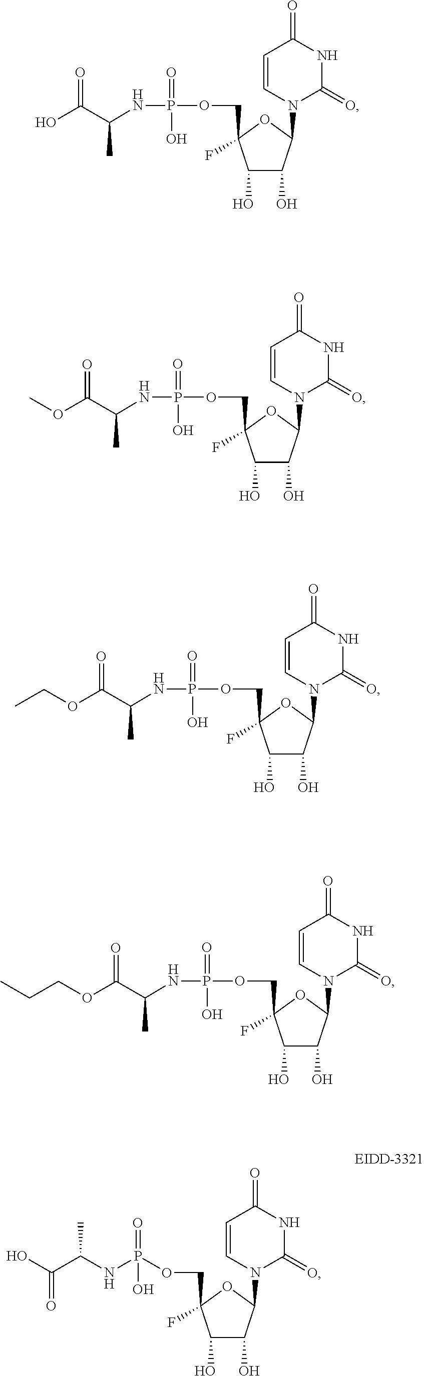 [SMILES: C[C@H](NP(=O)(O)OC[C@@]1(F)O[C@@H](n2ccc(=O)[nH]c2=O)[C@H](O)[C@@H]1O)C(=O)O ; COC(=O)[C@H](C)NP(=O)(O)OC[C@@]1(F)O[C@@H](n2ccc(=O)[nH]c2=O)[C@H](O)[C@@H]1O ; CCOC(=O)[C@H](C)NP(=O)(O)OC[C@@]1(F)O[C@@H](n2ccc(=O)[nH]c2=O)[C@H](O)[C@@H]1O ; CCCOC(=O)[C@H](C)NP(=O)(O)OC[C@@]1(F)O[C@@H](n2ccc(=O)[nH]c2=O)[C@H](O)[C@@H]1O ; C[C@H](NP(=O)(O)OC[C@@]1(F)O[C@@H](n2ccc(=O)[nH]c2=O)[C@H](O)[C@@H]1O)C(=O)O]

and combinations thereof.

In exemplified embodiments of Formula XXIX-Formula XXIXc, the compound is selected from:

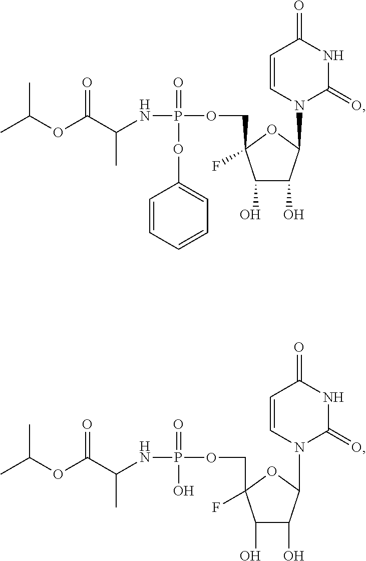 [SMILES: CC(C)OC(=O)C(C)NP(=O)(OC[C@@]1(F)O[C@@H](n2ccc(=O)[nH]c2=O)[C@H](O)[C@@H]1O)Oc1ccccc1 ; CC(C)OC(=O)C(C)NP(=O)(O)OCC1(F)OC(n2ccc(=O)[nH]c2=O)C(O)C1O]

and combinations thereof.

In exemplified embodiments of Formula XXIX-Formula XXIXc, the compound is selected from:

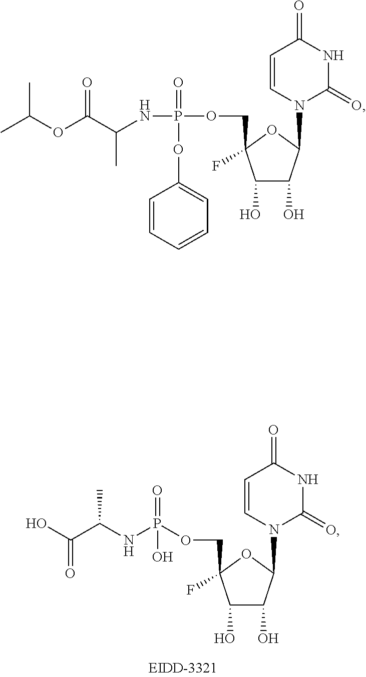 [SMILES: CC(C)OC(=O)C(C)NP(=O)(OC[C@@]1(F)O[C@@H](n2ccc(=O)[nH]c2=O)[C@H](O)[C@@H]1O)Oc1ccccc1 ; C[C@H](NP(=O)(O)OC[C@@]1(F)O[C@@H](n2ccc(=O)[nH]c2=O)[C@H](O)[C@@H]1O)C(=O)O]

and combinations thereof.

In exemplified embodiments of Formula XXIX-Formula XXIXc, the compound is a selected from:

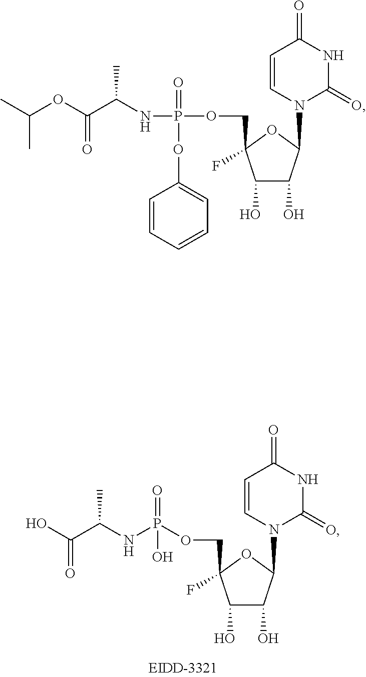 [SMILES: CC(C)OC(=O)[C@H](C)NP(=O)(OC[C@@]1(F)O[C@@H](n2ccc(=O)[nH]c2=O)[C@H](O)[C@@H]1O)Oc1ccccc1 ; C[C@H](NP(=O)(O)OC[C@@]1(F)O[C@@H](n2ccc(=O)[nH]c2=O)[C@H](O)[C@@H]1O)C(=O)O]

and combinations thereof.

In exemplified embodiments of Formula XXIX-Formula XXIXc, the compound is not:

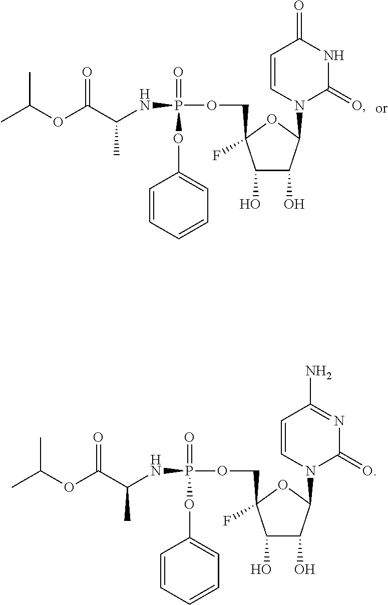 [SMILES: CC(C)OC(=O)[C@@H](C)N[P@@](=O)(OC[C@@]1(F)O[C@@H](n2ccc(=O)[nH]c2=O)[C@H](O)[C@@H]1O)Oc1ccccc1 ; CC(C)OC(=O)[C@H](C)N[P@@](=O)(OC[C@@]1(F)O[C@@H](n2ccc(N)nc2=O)[C@H](O)[C@@H]1O)Oc1ccccc1]

In certain embodiments, the disclosure relates to a compound of Formula XXX,

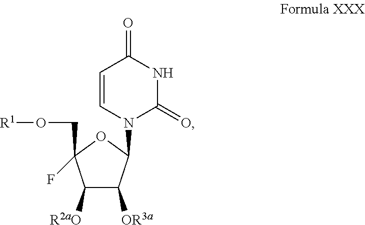

or a pharmaceutically acceptable salt thereof, wherein
R¹ is a structure represented by a formula selected from:

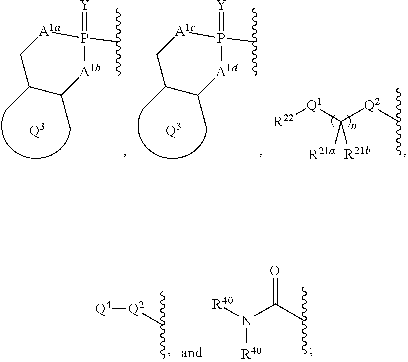

Q² is C(═O), O(C═O), S(C═O), NR⁴⁰(C═O), C(═S), O(C═S), S(C═S), or NR⁴⁰(C═S);
Q¹ is null, O, S, C(═O), O(C═O), S(C═O), NR⁴⁰(C═O), C(═S), O(C═S), S(C═S), NR⁴⁰(C═S), O(C═O)O, S(C═O)O, NR⁴⁰(C═O)O, S(C═O)S, NR⁴⁰(C═O)S, NR⁴⁰(C═O)NR⁴⁰, O(C═S)O, S(C═S)O, NR⁴⁰(C═S)O, S(C═S)S, NR⁴⁰(C═S)S, NR⁴⁰(C═S)NR⁴⁰, or NR⁴⁰;
Q³ is C₅-C₈cycloalkyl, C₆-C₁₂aryl, C₃-C₁₂heterocyclyl, or C₃-C₁₂heteroaryl; wherein Q⁴ can be optionally independently substituted with one or more, the same or different, R¹⁰;
Q⁴ is C₅-C₈ cycloalkyl, C₆-C₁₂aryl, C₃-C₁₂heterocyclyl, or C₃-C₁₂heteroaryl; wherein Q⁴ can be optionally independently substituted with one or more, the same or different, R¹⁰;
Y is O or S;
n is selected from 1, 2, and 3, preferably 1;
each of A^{1a}, A^{1b}, A^{1c}, and A^{1d} are independently selected from C, NR⁴⁰, S, and O;
R^{21a} is in each case independently hydrogen, deuterium, C₁-C₂₂ alkyl, C₁-C₂₂ alkylamino, (C₁-C₂₂ alkyl)₂amino, (CH₂)_{q}—(C₁-C₂₂ alkoxy), C₆-C₁₂aryl, C₃-C₁₂heteroaryl, C₃-C₁₂cycloalkyl, C₃-C₁₂heterocyclyl, or C₁-C₁₂ alkoxy;
R^{21b} is in each case hydrogen, deuterium, C₁-C₂₂ alkyl, C₁-C₂₂ alkylamino, (C₁-C₂₂ alkyl)₂amino, (CH₂)_{q}—(C₁-C₂₂ alkoxy), C₆-C₁₂aryl, C₃-C₁₂cycloalkyl, C₃-C₁₂heteroaryl, C₃-C₁₂heterocyclyl, or C₁-C₁₂ alkoxy;
R²² is in each case hydrogen, deuterium, C₁-C₂₂ alkyl, C₁-C₂₂ alkylamino, (C₁-C₂₂ alkyl)₂amino, (CH₂)_{q}—(C₁-C₂₂ alkoxy), C₆-C₁₂aryl, C₃-C₁₂cycloalkyl, C₃-C₁₂heterocyclyl, C₃-C₁₂heteroaryl, or C₁-C₁₂ alkoxy;
wherein each of R^{21a}, R^{21b}, and R²² can be optionally substituted with one or more, the same or different, R¹⁰;
R⁴⁰ is in each case independently selected from hydrogen and C₁-C₆alkyl, optionally substituted one more times by R¹⁰;
wherein any two or more of R^{21a}, R^{21b}, R²², and R⁴⁰ may together form a ring, for example a C₃-C₈cycloalkyl ring, a C₆-C₁₂aryl ring, a C₃-C₈heteroaryl ring, or a C₂-C₈heterocyclyl ring;
each of R^{2a} and R^{3a} is independently selected from hydrogen, C₁-C₆alkyl, (C═O)C₁-C₆alkyl, (C═O)NR⁴⁰C₁-C₆alkyl, (C═O)N(C₁-C₆ alkyl)₂, (C═O)OC₁-C₆alkyl; wherein said alkyl groups are optionally independently substituted with one or more, the same or different, R¹⁰;
   wherein R^{2a} and R^{3a} can together form a 5-7 membered heterocyclic ring;
R¹⁰ is in each case independently deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
lipid is independently a C₁₁-C₂₂alkyl, C₁₁-C₂₂alkoxy, or aryl substituted with an C₆-C₁₈ alkyl group.

In certain implementations, when Q³ represents a phenyl ring, then A^{1a} and A^{1b} are not both O.

In some implementations, each of R^{2a} and R^{3a} are (C═O)C₁-C₆alkyl or (C═O)OC₁-C₆alkyl; wherein said alkyl groups are optionally independently substituted with one or more, the same or different, R¹⁰. In some implementations R^{2a} and R^{3a} together are C(═O), C(═S), or C(CH₃)₂.

In some implementations, each of R^{2a} and R^{3a} are (C═O)C₁-C₃alkyl or (C═O)OC₁-C₃alkyl; wherein said alkyl groups are optionally independently substituted with one or more, the same or different, R¹⁰; or R^{2a} and R^{3a} together are C(═O), C(═S), or C(CH₃)₂.

In certain implementations, R¹⁰ is F, Cl, OC₁-C₃alkyl, CF₃, OCF₃, or phenyl, wherein said phenyl is optionally substituted one or more times by F, Cl, OC₁-C₃alkyl, CF₃, OCF₃.

In certain implementations each of R^{21a}, R^{21b}, and R²² are independently selected from hydrogen, deuterium, C₁-C₁₂ alkyl, C₁-C₁₂ alkylamino, (C₁-C₁₂ alkyl)₂amino, (CH₂)_{q}—(C₁-C₁₂ alkoxy), C₃-C₁₂cycloalkyl, C₃-C₁₂heterocyclyl, and C₁-C₁₂ alkoxy; wherein q is an integer selected from 1, 2, and 3.

In some implementations, R¹ is:

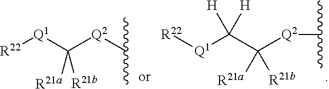

In certain implementations Q¹ is null. In some implementations, Q¹ is null and R²² is H.

In some implementations, Q¹ is null, R²² is H, and each of R^{21a} and R^{21b} are C₁-C₆alkyl, optionally substituted by R¹⁰. In some implementations, Q¹ is null, R^{21b} is H, and each of R^{21a} and R²² are C₁-C₆alkyl, optionally substituted by R¹⁰. In some implementations, Q¹ is null, R²² is H, and each of R^{21a} and R^{21b} are methyl. In some implementations, Q¹ is null, R²² is C₁-C₆alkyl, optionally substituted by R¹⁰, and each of R^{21a} and R^{21b} are H.

In some implementations, Q¹ is O or NH. In some implementations, Q¹ is O or NH and Q² is C(═O), O(C═O), NR⁴⁰(C═O), or NR⁴⁰(C═S). In some implementations, Q¹ is O or NH and Q² is C(═O), O(C═O), NR⁴⁰(C═O), or NR⁴⁰(C═S), and R²², R^{21a} and R^{21b} are independently selected from H and C₁-C₆alkyl, optionally substituted by R¹⁰. In some implementations,

In some implementations, Q¹ is C(═O), O(C═O), NR⁴⁰(C═O), C(═S), O(C═S), NR⁴⁰(C═S), O(C═O)O, S(C═O)O, NR⁴⁰(C═O)O, or NR⁴⁰. In some implementations, Q¹ is C(═O), O(C═O), NR⁴⁰(C═O), C(═S), O(C═S), NR⁴⁰(C═S), O(C═O)O, S(C═O)O, NR⁴⁰(C═O)O, or NR⁴⁰ and Q² is C(═O), O(C═O), NR⁴⁰(C═O), or NR⁴⁰(C═S). In some implementations, Q¹ is C(═O), O(C═O), NR⁴⁰(C═O), C(═S), O(C═S), NR⁴⁰(C═S), O(C═O)O, S(C═O)O, NR⁴⁰(C═O)O, or NR⁴⁰, Q² is C(═O), O(C═O), NR⁴⁰(C═O), or NR⁴⁰(C═S), and R²², R^{21a} and R^{21b} are independently selected from H and C₁-C₆alkyl, optionally substituted by R¹⁰.

In some embodiments R¹ can be

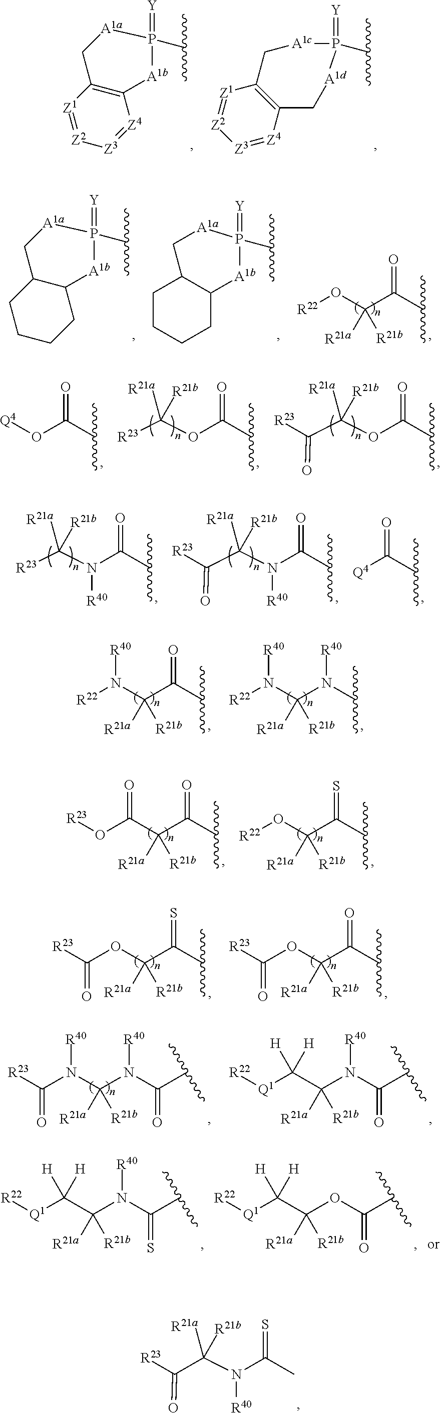

wherein n is 1, and
R²³ is hydrogen, C₁-C₁₂ alkyl, C₁-C₁₂ alkylamino, (C₁-C₁₂ alkyl)₂amino, (CH₂)_{q}—(C₁-C₁₂ alkoxy), C₃-C₁₂cycloalkyl, C₆-C₁₂aryl, C₃-C₁₂heterocyclyl, and C₁-C₁₂ alkoxy; wherein q is an integer selected from 1, 2, and 3, wherein R²³ is optionally substituted one or more time by R¹⁰, and wherein any two or more of R²³, R^{21a}, R^{21b}, and R⁴⁰ may form a ring.
Z¹ is N or C—R^{20a};
Z² is N or C—R^{20b};
Z³ is N or C—R^{20c};
Z⁴ is N or C—R^{20d};
R^{20a}, R^{20b}, R^{20c}, and R^{20d} are each independently selected from hydrogen, deuterium, hydroxyl, amino, cyano, halogen, C₁-C₆alkyl, OC₁-C₆alkyl, (C═O)C₁-C₆alkyl, (C═O)NR⁴⁰C₁-C₆alkyl, (C═O)N(C₁-C₆ alkyl)₂, (C═O)OC₁-C₆ alkyl, and lipid, wherein said alkyl groups are optionally, independently substituted with one or more R¹⁰;

In exemplified embodiments of Formula XXX, the compound has a structure represented by a formula selected from:

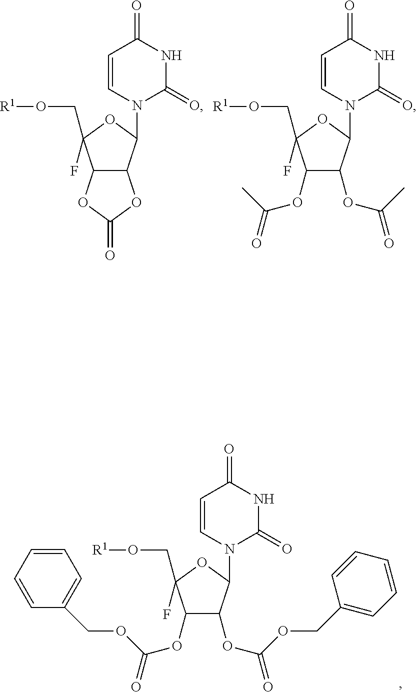

and combinations thereof.

In exemplified embodiments of Formula XXX, the compound has a structure represented by a formula:

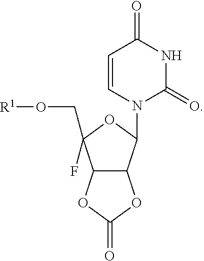

In exemplified embodiments of Formula XXX, the compound has a structure represented by a formula:

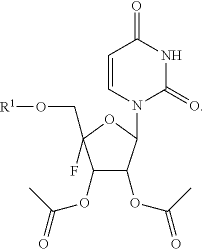

In exemplified embodiments of Formula XXX, R^{2a} and R^{3a} are both (C═O)OC₁-C₆ alkyl substituted by aryl or heteroaryl, preferably phenyl or pyridinyl. In some embodiments, the compound has a structure represented by a formula:

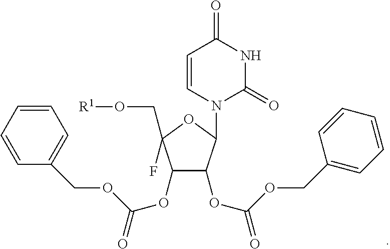

In some implementations, R¹ is:

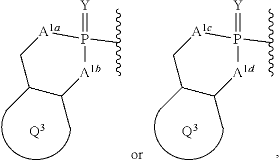

In some implementations, A^{1a}/A^{1c} is NH and A^{1b}/A^{1d} is O, A^{1a}/A^{1c} is O and A^{1b}/A^{1d} is NH, or A^{1a}/A^{1c} is CH₂ and A^{1b}/A^{1d} is O. In some implementations, Y is O.

In certain implementations Q³ is a saturated cycloalkyl ring, optionally substituted one or more times, independently, by R¹⁰. In some implementations Q³ is a saturated cycloheptyl, cyclohexyl, or cyclopentyl ring. In some implementations Q³ is a saturated cycloheptyl, cyclohexyl, or cyclopentyl ring, wherein said ring is not further substituted. In some implementations Q³ is a saturated cycloheptyl, cyclohexyl, or cyclopentyl ring, wherein said ring is substituted by at least one R¹⁰. In such implementations, preferably R¹⁰ is C₁-C₃alkyl, C₁-C₃alkoxy, C₁-C₃ haloalkyl, C₁-C₃ haloalkoxy, or halo, preferably fluoro or chloro.

In certain implementations Q³ is a phenyl ring, optionally substituted one or more times, independently, by R¹⁰. In some implementations Q³ is a phenyl ring, wherein said ring is not further substituted. In some implementations Q³ is a phenyl ring, wherein said ring is substituted by at least one R¹⁰. In such implementations, preferably R¹⁰ is C₁-C₃alkyl, C₁-C₃alkoxy, C₁-C₃ haloalkyl, C₁-C₃ haloalkoxy, or halo, preferably fluoro or chloro.

In certain implementations Q³ is a pyridinyl ring, optionally substituted one or more times, independently, by R¹⁰. In some implementations Q³ is a pyridinyl ring, wherein said ring is not further substituted. In some implementations Q³ is a pyridinyl ring, wherein said ring is substituted by at least one R¹⁰. In such implementations, preferably R¹⁰ is C₁-C₃alkyl, C₁-C₃alkoxy, C₁-C₃ haloalkyl, C₁-C₃ haloalkoxy, or halo, preferably fluoro or chloro.

In exemplified embodiments of Formula XXX, R¹ is a structure represented by a formula selected from:

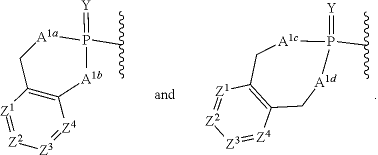

In exemplified embodiments of Formula XXX, R¹ is a group having a structure represented by a formula:

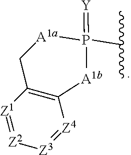

In exemplified embodiments of Formula XXX, R¹ is a structure represented by a formula selected from:

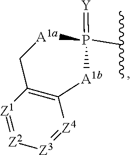

wherein the compound is at least 75 mol %, at least 85 mol %, at least 90 mol %, at least 95 mol %, or at least 99 mol % the depicted stereoisomer, and preferably Y is O.

In exemplified embodiments of Formula XXX, R¹ is a structure represented by a formula selected from:

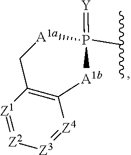

wherein the compound is at least 75 mol %, at least 85 mol %, at least 90 mol %, at least 95 mol %, or at least 99 mol % the depicted stereoisomer, and preferably Y is O.

In exemplified embodiments R¹ is:

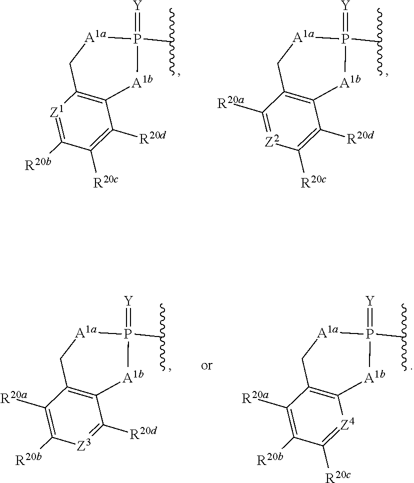

In certain implementations, Z¹, Z², Z³, Z⁴ are N or CH, and preferably Y is O.

In exemplified embodiments of Formula XXX, R¹ is a group having a structure represented by a formula:

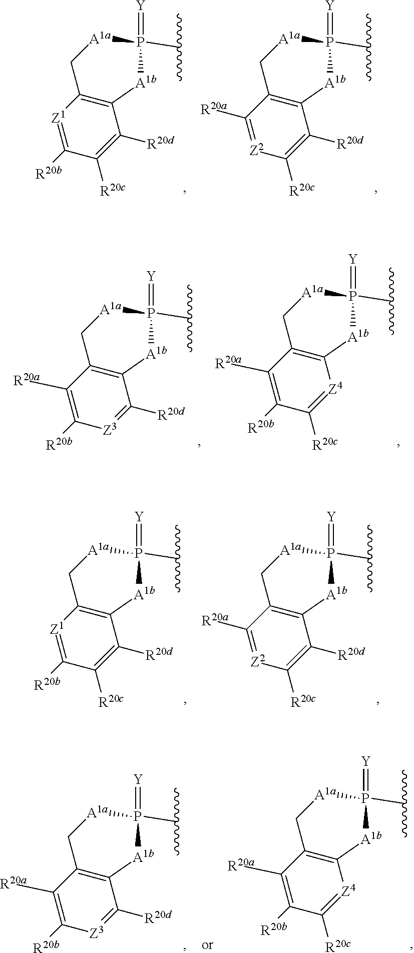

wherein the compound is at least 75 mol %, at least 85 mol %, at least 90 mol %, at least 95 mol %, or at least 99 mol % the depicted stereoisomer. In certain implementations, Z¹, Z², Z³, Z⁴ are N or CH, and Y is O.

In exemplified embodiments R¹ is:

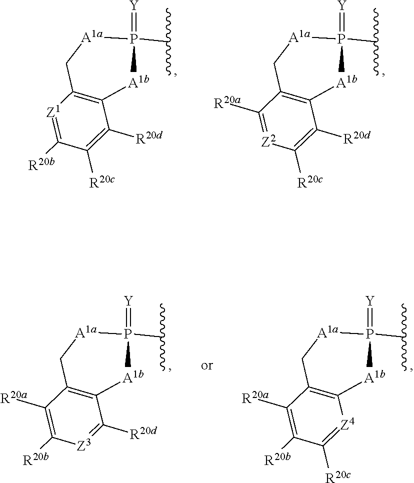

wherein the compound is at least 75 mol %, at least 85 mol %, at least 90 mol %, at least 95 mol %, or at least 99 mol % the depicted stereoisomer. In certain implementations, Z¹, Z², Z³, Z⁴ are N or CH and Y is O.

In exemplified embodiments of Formula XXX, R¹ is:

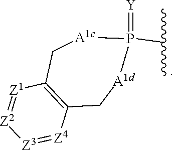

In certain implementations, one of Z¹, Z², Z³, and Z⁴ is N. In certain implementations, one of Z¹, Z², Z³, and Z⁴ is N and the other three are CH. In some implementations,
Z¹ is N, and Z², Z³, and Z⁴ are CH;
Z² is N, and Z², Z³, and Z⁴ are CH;
Z³ is N, and Z¹, Z², and Z⁴ are CH; or
Z⁴ is N, and Z¹, Z², and Z³ are CH.

In exemplified embodiments of Formula XXX, at least one of R^{20a}, R^{20b}, R^{20c}, and R^{20d} is not hydrogen. In exemplified embodiments of Formula XXX, R^{20a}, R^{20b}, R^{20c}, and R^{20d} are independently selected from hydrogen, deuterium, C₁-C₃alkyl, C₁-C₃alkoxy, C₁-C₃haloalkyl, C₁-C₃haloalkoxy, hydroxyl, amino, cyano, F, Cl, Br, and I.

In exemplified embodiments of Formula XXX, R^{20a}, R^{20b}, R^{20c}, and R^{20d} are independently selected from hydrogen, C₁-C₃alkyl, C₁-C₃alkoxy, C₁-C₃haloalkyl, C₁-C₃haloalkoxy, and halogen.

In exemplified embodiments of Formula XXX, R^{20a}, R^{20b}, R^{20c}, and R^{20d} are independently selected from hydrogen, CH₃, OCH₃, CF₃, OCF₃, and halogen.

In exemplified embodiments of Formula XXX, halogen is selected from Cl, F, and I.

In exemplified embodiments of Formula XXX, halogen is Cl or F.

In exemplified embodiments of Formula XXX, halogen is Cl.

In exemplified embodiments of Formula XXX, at least one of R^{20a}, R^{20b}, R^{20c}, and R^{20d} is halogen.

In exemplified embodiments of Formula XXX, one or two of R^{20a}, R^{20b}, R^{20c}, and R^{20d} is halogen and the other are hydrogen.

In exemplified embodiments of Formula XXX, one or two of R^{20a}, R^{20b}, R^{20c}, and R^{20d} is methyl and the other are hydrogen.

In exemplified embodiments of Formula XXX, three of R^{20a}, R^{20b}, R^{20c}, and R^{20d} is methyl and the other are hydrogen.

In exemplified embodiments of Formula XXX, one or two of R^{20a}, R^{20b}, R^{20c}, and R^{20d} is methoxy and the other are hydrogen.

In exemplified embodiments of Formula XXX, one or two of R^{20e}, R^{20f}, R^{20g}, R^{20h}, and R²⁰ⁱ are C₁ and the other are hydrogen.

In exemplified embodiments of Formula XXX, one or two of R^{20e}, R^{20f}, R^{20g}, R^{20h}, and R²⁰ⁱ are F and the other are hydrogen.

In some implementations, R¹ can be:

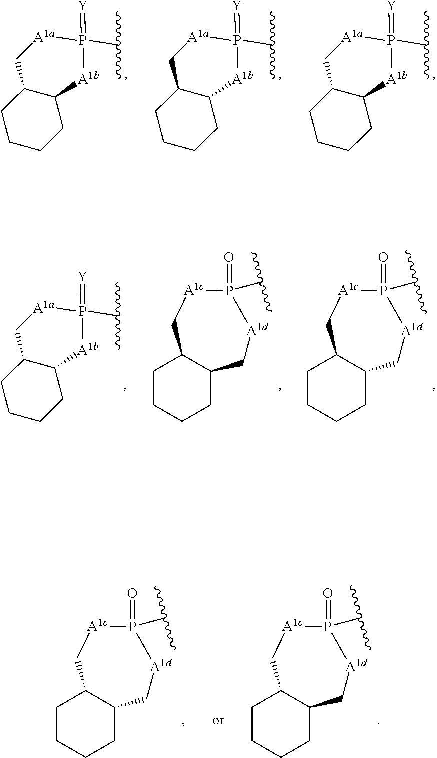

In some implementations, A^{1a}/A^{1c} is NH and A^{b1}/A^{1d} is O, A^{1a}/A^{1c} is O and A^{1b}/A^{1d} is NH, or A^{1a}/A^{1c} is CH₂ and A^{1b}/A^{1d} is O

In exemplified embodiments of Formula XXX, the compound has a structure represented by a formula selected from:

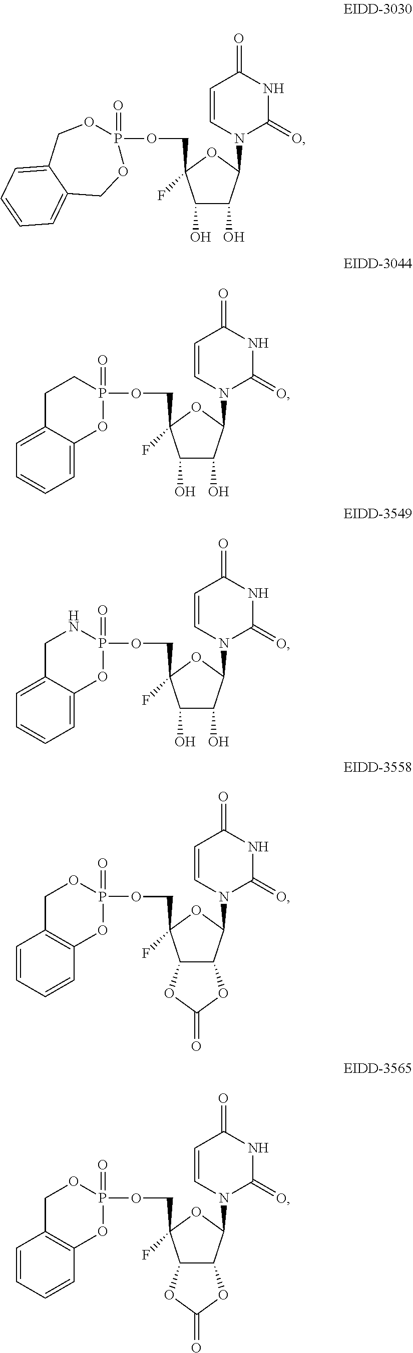 [SMILES: O=c1ccn([C@@H]2O[C@](F)(COP3(=O)OCc4ccccc4CO3)[C@@H](O)[C@H]2O)c(=O)[nH]1 ; O=c1ccn([C@@H]2O[C@](F)(COP3(=O)CCc4ccccc4O3)[C@@H](O)[C@H]2O)c(=O)[nH]1 ; O=c1ccn([C@@H]2O[C@](F)(COP3(=O)NCc4ccccc4O3)[C@@H](O)[C@H]2O)c(=O)[nH]1 ; O=C1O[C@H]2[C@H](n3ccc(=O)[nH]c3=O)O[C@](F)(COP3(=O)OCc4ccccc4O3)[C@H]2O1 ; O=C1O[C@H]2[C@H](n3ccc(=O)[nH]c3=O)O[C@](F)(COP3(=O)OCc4ccccc4O3)[C@H]2O1]

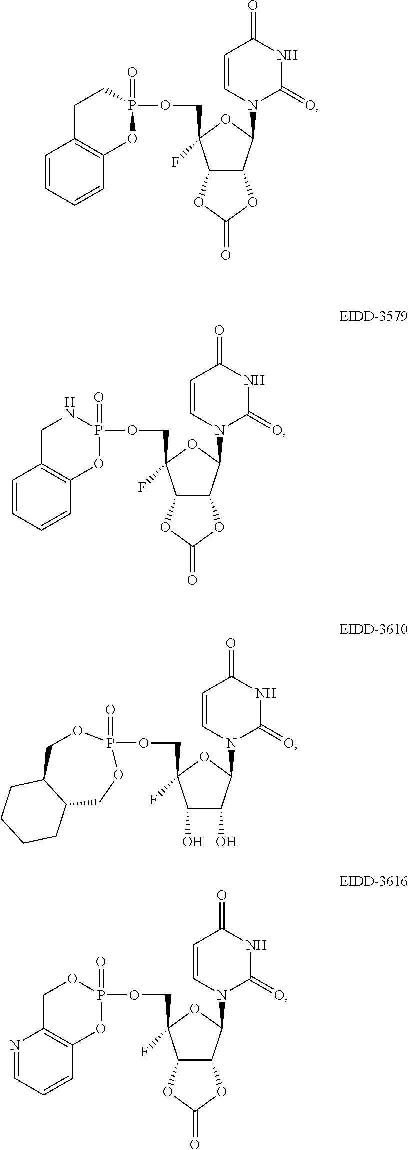 [SMILES: O=C1O[C@H]2[C@H](n3ccc(=O)[nH]c3=O)O[C@](F)(CO[P@@]3(=O)CCc4ccccc4O3)[C@H]2O1 ; O=C1O[C@H]2[C@H](n3ccc(=O)[nH]c3=O)O[C@](F)(COP3(=O)NCc4ccccc4O3)[C@H]2O1 ; O=c1ccn([C@@H]2O[C@](F)(COP3(=O)OC[C@@H]4CCCC[C@H]4CO3)[C@@H](O)[C@H]2O)c(=O)[nH]1 ; O=C1O[C@H]2[C@H](n3ccc(=O)[nH]c3=O)O[C@](F)(COP3(=O)OCc4ncccc4O3)[C@H]2O1]

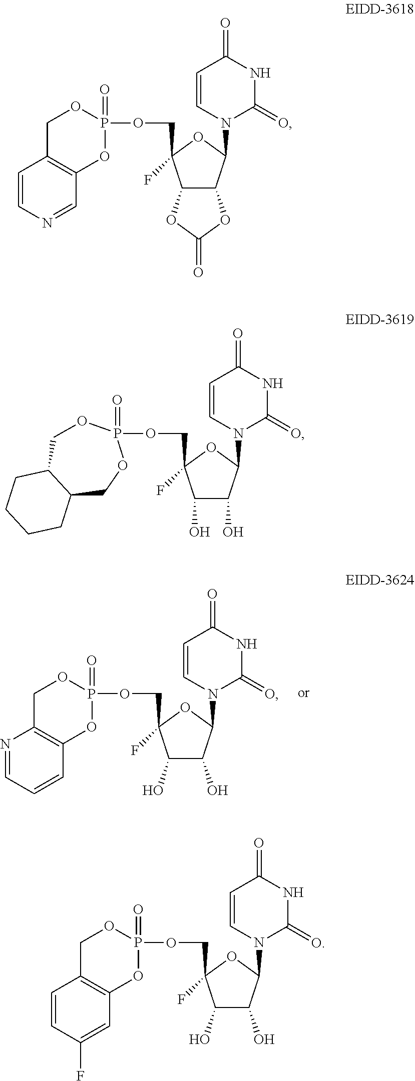 [SMILES: O=C1O[C@H]2[C@H](n3ccc(=O)[nH]c3=O)O[C@](F)(COP3(=O)OCc4ccncc4O3)[C@H]2O1 ; O=c1ccn([C@@H]2O[C@](F)(COP3(=O)OC[C@H]4CCCC[C@@H]4CO3)[C@@H](O)[C@H]2O)c(=O)[nH]1 ; O=c1ccn([C@@H]2O[C@](F)(COP3(=O)OCc4ncccc4O3)[C@@H](O)[C@H]2O)c(=O)[nH]1 ; O=c1ccn([C@@H]2O[C@](F)(COP3(=O)OCc4ccc(F)cc4O3)[C@@H](O)[C@H]2O)c(=O)[nH]1]

In exemplified embodiments of Formula XXX, the compound has a structure represented by a formula selected from:

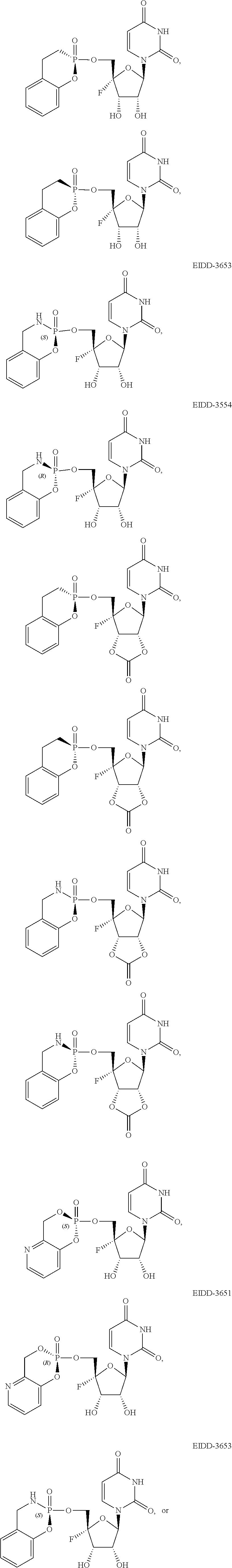

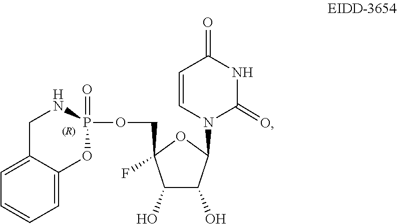

wherein the compound is at least 75 mol %, at least 85 mol %, at least 90 mol %, at least 95 mol %, or at least 99 mol % the depicted stereoisomer at the phosphorous atom.

In exemplified embodiments of Formula XXX, R¹ is:

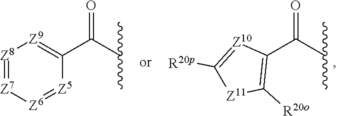

wherein Z⁵ is selected from N and C—R^{20e};
wherein Z⁶ is selected from N and C—R^{20f};
wherein Z⁷ is selected from N and C—R^{20g};
wherein Z⁸ is selected from N and C—R^{20h};
wherein Z⁹ is selected from N and C—R²⁰ⁱ;
provided that no more than three of Z⁵, Z⁶, Z⁷, Z⁸, and Z⁹ are N;
R^{20e}, R^{20f}, R^{20g}, R^{20h}, R²⁰ⁱ, R^{20o}, sand R^{20p} are each independently selected from hydrogen, deuterium, hydroxyl, amino, cyano, halogen, C₁-C₆ alkyl, OC₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl or lipid; wherein said alkyl groups are optionally independently substituted with one or more, the same or different, R¹⁰;
wherein two of R^{20e}, R^{20f}, R^{20g}, R^{20h}, and R²⁰ⁱ may together form a ring, for example an optionally substituted 5- to 7-membered cycloalkyl or heterocyclyl ring;
Z¹⁰ is selected from N and C—R^{20j};
Z¹¹ is selected from N—R^{20k} and C—R^{20l}R^{20m};
R^{20j}, R^{20l}, and R^{20m} are each independently selected from hydrogen, deuterium, hydroxyl, amino, cyano, halogen, C₁-C₆ alkyl, OC₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid; wherein said alkyl groups are optionally independently substituted with one or more, the same or different, R¹⁰; wherein any two of more of R^{20j}, R^{20k}, R^{20l}, R^{20m}, R^{20o}, and R^{20p} may together form a ring;
R^{20k} is hydrogen or C₁-C₆ alkyl optionally independently substituted with one or more, the same or different, R¹⁰;
R⁴⁰ is in each case independently selected from hydrogen and C₁-C₆alkyl, optionally 5 substituted one more times by R¹⁰.

In exemplified embodiments of Formula XXX, at least one of R^{20e}, R^{20f}, R^{20g}, R^{20h}, and R²⁰ⁱ is not hydrogen. In exemplified embodiments of Formula XXX, R^{20e}, R^{20f}, R^{20g}, R^{20h}, and R²⁰ⁱ are independently selected from hydrogen, deuterium, C₁-C₃alkyl, C₁-C₃alkoxy, C₁-C₃haloalkyl, C₁-C₃haloalkoxy, hydroxyl, amino, cyano, F, Cl, Br, and I.

In exemplified embodiments of Formula XXX, R^{20e}, R^{20f}, R^{20g}, R^{20h}, and R²⁰ⁱ are independently selected from hydrogen, C₁-C₃alkyl, C₁-C₃alkoxy, C₁-C₃haloalkyl, C₁-C₃haloalkoxy, and halogen.

In exemplified embodiments of Formula XXX, R^{20e}, R^{20f}, R^{20g}, R^{20h}, and R²⁰ⁱ are independently selected from hydrogen, CH₃, OCH₃, CF₃, OCF₃, and halogen.

In exemplified embodiments of Formula XXX, halogen is selected from Cl, F, and I.

In exemplified embodiments of Formula XXX, halogen is Cl or F.

In exemplified embodiments of Formula XXX, halogen is Cl.

In exemplified embodiments of Formula XXX, at least one of R^{20e}, R^{20f}, R^{20g}, R^{20h}, and R²⁰ⁱ is halogen.

In exemplified embodiments of Formula XXX, one or two of R^{20e}, R^{20f}, R^{20g}, R^{20h}, and R²⁰ⁱ is halogen and the other are hydrogen.

In exemplified embodiments of Formula XXX, one or two of R^{20e}, R^{20f}, R^{20g}, R^{20h}, and R²⁰ⁱ is methyl and the other are hydrogen.

In exemplified embodiments of Formula XXX, three of R^{20e}, R^{20f}, R^{20g}, R^{20h}, and R²⁰ⁱ is methyl and the other are hydrogen.

In exemplified embodiments of Formula XXX, one or two of R^{20e}, R^{20f}, R^{20g}, R^{20h}, and R²⁰ⁱ is methoxy and the other are hydrogen.

In exemplified embodiments of Formula XXX, one or two of R^{20e}, R^{20f}, R^{20g}, R^{20h}, and R²⁰ⁱ are Cl and the other are hydrogen.

In exemplified embodiments of Formula XXX, one or two of R^{20e}, R^{20f}, R^{20g}, R^{20h}, and R²⁰ⁱ are F and the other are hydrogen.

In exemplified embodiments of Formula XXX, R¹ is a structure represented by a formula selected from:

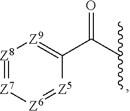

wherein Z⁵ is selected from N and C—R^{20e};
wherein Z⁶ is selected from N and C—R^{20f};
wherein Z⁷ is selected from N and C—R^{20g};
wherein Z⁸ is selected from N and C—R^{20h};
wherein Z⁹ is selected from N and C—R²⁰ⁱ;
provided that no more than three of Z⁵, Z⁶, Z⁷, Z⁸, and Z⁹ are N;
wherein R^{20e}, R^{20f}, R^{20g}, R^{20h}, and R²⁰ⁱ are each independently selected from hydrogen, deuterium, hydroxyl, amino, cyano, halogen, C₁-C₆ alkyl, OC₁-C₆ alkyl (C═O)C₁-C₆ alkyl, (C═O)NR⁴⁰C₁-C₆ alkyl, (C═O)N(C₁-C₆ alkyl)₂, (C═O)OC₁-C₆ alkyl, or lipid;
wherein R^{20e}, R^{20f}, R^{20g}, R^{20h}, and R²⁰ⁱ can each be optionally independently substituted with one or more, the same or different, R¹⁰;
wherein R⁴⁰ is in each case independently selected from hydrogen and C₁-C₆alkyl, optionally substituted one more times by R¹⁰; and
wherein two of R^{20e}, R^{20f}, R^{20g}, R^{20h}, and R²⁰ⁱ may together form a ring, for example an optionally substituted 5- to 7-membered cycloalkyl or heterocyclyl ring;
   wherein lipid is independently a C₁₁-C₂₂alkyl, C₁₁-C₂₂alkoxy, or aryl substituted with an C₆-C₁₈ alkyl group.

In certain embodiments, Z¹⁰ is CH and Z¹¹ is NH. In some embodiments Z¹⁰ is N and Z¹¹ is NH.

In exemplified embodiments of Formula XXX, the compound has a structure represented by a formula selected from:

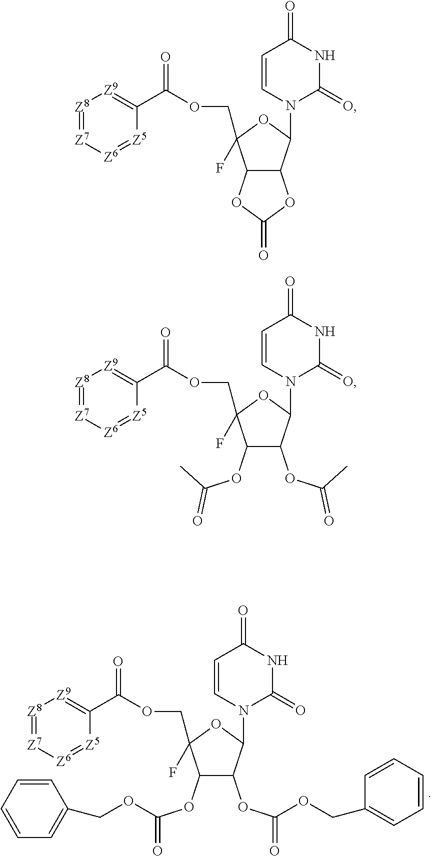

In exemplified embodiments of Formula XXX, the compound has a structure represented by a formula:

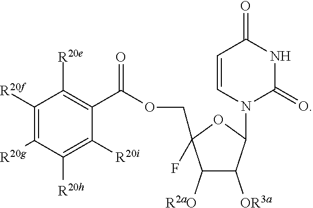

In exemplified embodiments of Formula XXX, the compound has a structure represented by a formula:

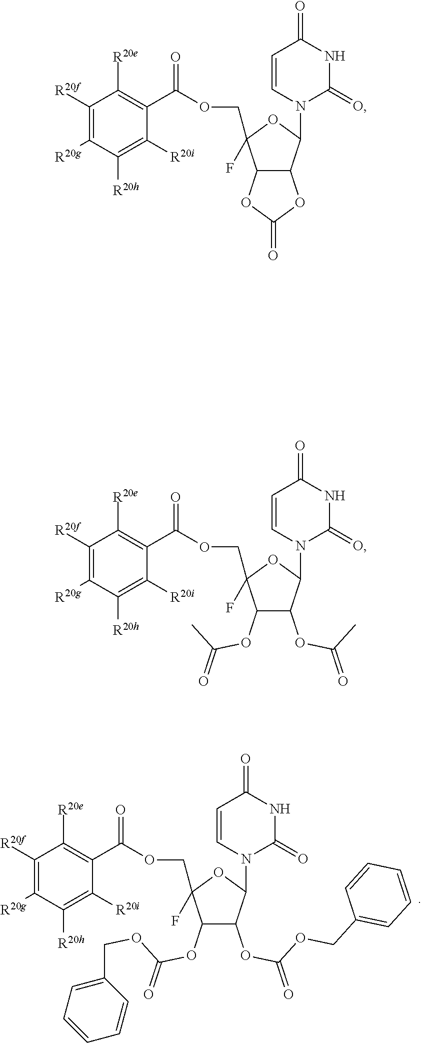

In exemplified embodiments of Formula XXX, at least one of R^{20e}, R^{20f}, R^{20g}, R^{20h}, and R²⁰ⁱ is not hydrogen. In exemplified embodiments of Formula XXX, R^{20e}, R^{20f}, R^{20g}, R^{20h}, and R²⁰ⁱ are each independently selected from hydrogen, deuterium, hydroxyl, amino, cyano, halogen, and combinations thereof.

In exemplified embodiments of Formula XXX, R^{20e}, R^{20f}, R^{20g}, R^{20h}, and R²⁰ⁱ are each independently selected from hydrogen, halogen, and combinations thereof.

In certain implementations, R^{20h} and R²⁰ⁱ together form a six-member ring. In certain implementations, R^{20h} and R²⁰ⁱ together form a six-member carbocyclic ring. In certain implementations, R^{20h} and R²⁰ⁱ together form a six-member heterocyclic ring.

In certain implementations, R^{20h} and R^{20g} together form a six-member ring. In certain implementations, R^{20h} and R^{20g} together form a six-member carbocyclic ring. In certain implementations, R^{20h} and R^{20g} together form a six-member heterocyclic ring.

In exemplified embodiments of Formula XXX, halogen is selected from —Cl, —F, and —I.

In exemplified embodiments of Formula XXX, halogen is —Cl.

In exemplified embodiments of Formula XXX, at least one of R^{20e}, R^{20f}, R^{20g}, R^{20h}, and R²⁰ⁱ is halogen.

In exemplified embodiments of Formula XXX, one or two of R^{20e}, R^{20f}, R^{20g}, R^{20h}, and R²⁰ⁱ is halogen and the other are hydrogen.

In exemplified embodiments of Formula XXX, one or two of R^{20e}, R^{20f}, R^{20g}, R^{20h}, and R²⁰ⁱ is Cl and the other are hydrogen.

In some implementations, Q⁴ is:

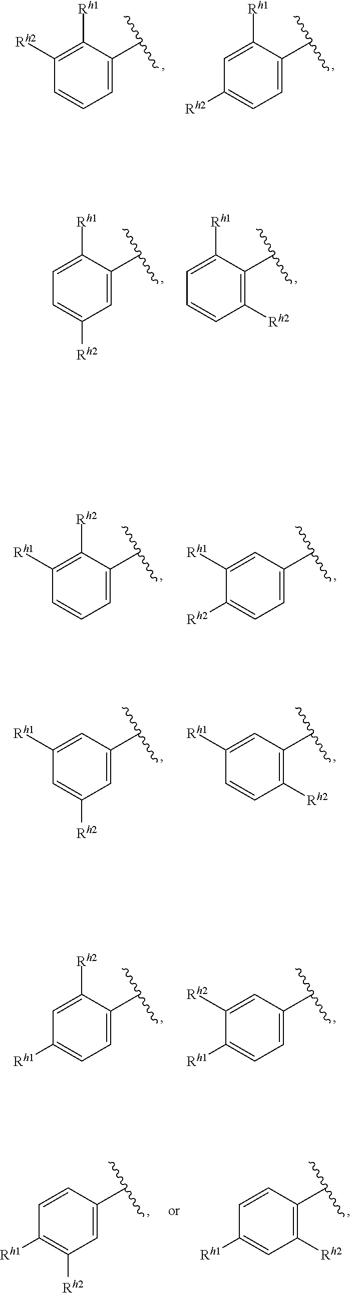

wherein R^{h1} is CH₃, OCH₃, CF₃, OCF₃, I, Cl or F, and R^{h2} is H, CH₃, OCH₃, CF₃, OCF₃, I, Cl, or F. In such implementations, Q² can be C(═O), C(═S), NH(C═O), or NH(C═S).

In exemplified embodiments of Formula XXX, the compound is selected from a structure having a formula:

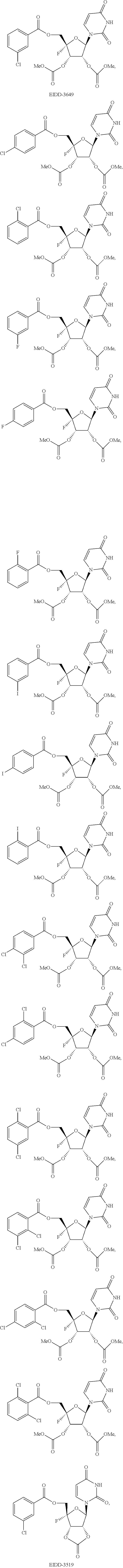

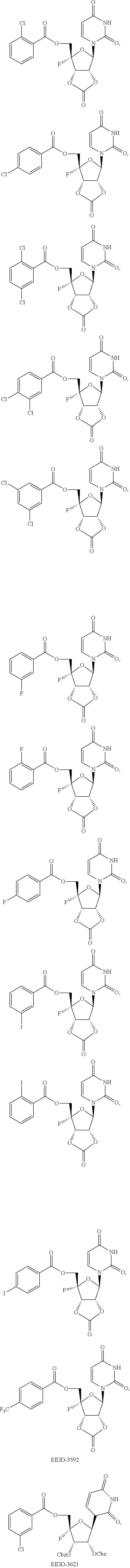

and combinations thereof.

In exemplified embodiments of Formula XXX, R¹ is a structure represented by a formula:

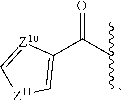

wherein Z¹⁰ is selected from N and C—R^{20j};
wherein Z¹¹ is selected from N—R^{20k} and C—R^{20l}R^{20m};
wherein R^{20j}, R^{20l}, and R^{20m} are each independently selected from hydrogen, deuterium, hydroxyl, amino, cyano, halogen, —C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid;
wherein R^{20j}, R^{20l}, and R^{20m} can each be optionally independently substituted with one or more, the same or different, R¹⁰; and
wherein R^{20k} is selected from hydrogen, deuterium, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl;
wherein R⁴⁰ is in each case independently selected from hydrogen and C₁-C₆alkyl, optionally substituted one more times by R¹⁰.

In exemplified embodiments of Formula XXX, R¹ is a structure represented by a formula selected from:

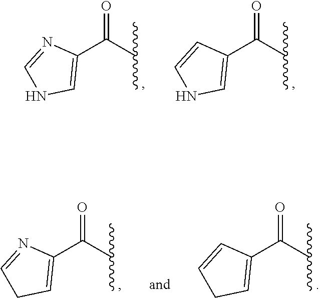

In exemplified embodiments of Formula XXX, R¹ is a structure represented by a formula selected from:

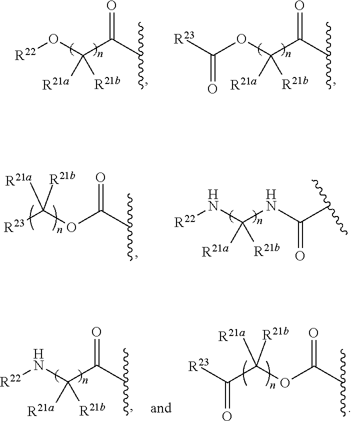

In exemplified embodiments of Formula XXX, R¹ is a structure represented by a formula selected from:

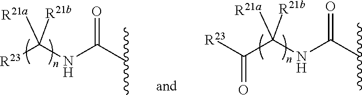

In exemplified embodiments of Formula XXX, each of R^{21a}, R^{21b}, and R²³ is independently selected from hydrogen, deuterium, C₁-C₆ alkyl, C₁-C₆ alkylamino, (C₁-C₆ alkyl)₂amino, and C₁-C₆ alkoxy.

In certain implementations, each of R^{21a}, R^{21b}, and R²³ is independently hydrogen, deuterium, C₁-C₈alkyl, C₁-C₈alkylamino, (C₁-C₈ alkyl)₂amino, and C₁-C₈alkoxy, wherein R^{21a}, R^{21b}, and R²³ are optionally, independently substituted by R¹⁰.

In some implementations, R^{21a} and R^{21b} together form a C₃-C₇cycloalkyl or C₁-C₇heterocyclyl ring.

In some implementations R^{21a} and R²³ together form a ring, e.g., a lactone when R¹ is:

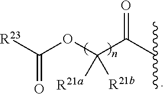

In some implementations, R^{21a} and R²³ together define a butyrolactone, a valerolactone, or a caprolactone, optionally substituted one or more times by R¹⁰.

In some implementations, R^{21a} and R²² together form a ring, e.g., a cyclic ether when R¹ is:

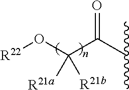

In some implementations, R²² and R²³ together define an oxirane (3-membered ring), an oxetane (4-membered ring), an oxolane (5-membered ring), an oxane (6-membered ring), an oxepane (7-membered ring), optionally substituted one or more times by R¹⁰. In certain implementations, Q¹ is NH, and R^{21a} and R²² can together form a heterocyclyl, analogously as above, for example aziridine, azetidine, pyrrolidine, piperidine, or azepane, optionally substituted one or more times by R¹⁰.

In exemplified embodiments of Formula XXX, each of R^{21a}, R^{21b}, and R²³ is independently selected from hydrogen, deuterium, C₁-C₃ alkyl, C₁-C₃ alkylamino, (C₁-C₃ alkyl)₂amino, and C₁-C₃ alkoxy.

In exemplified embodiments of Formula XXX, R²² is selected from hydrogen and C₁-C₂₀ alkyl. For example, R²² can be C₁₂-C₂₂alkyl.

In exemplified embodiments of Formula XXX, R²² is selected from hydrogen and C₁-C₁₈ alkyl; R²² is selected from hydrogen and C₁-C₁₆ alkyl; R²² is selected from hydrogen and C₁-C₁₄ alkyl; R²² is selected from hydrogen and C₁-C₁₂ alkyl; R²² is selected from hydrogen and C₁-C₁₀ alkyl; R²² is selected from hydrogen and C₁-C₈ alkyl; or R²² is selected from hydrogen and C₁-C₆ alkyl.

In exemplified embodiments of Formula XXX, R⁴⁰ is selected from hydrogen and C₁-C₃ alkyl.

In exemplified embodiments of Formula XXX, R⁴⁰ is selected from hydrogen, methyl, and ethyl.

In exemplified embodiments of Formula XXX, R⁴⁰ is selected from hydrogen and methyl.

In exemplified embodiments of Formula XXX, R⁴⁰ is hydrogen,

In exemplified embodiments of Formula XXX, R⁴⁰ is methyl.

In certain embodiments, the disclosure relates to a compound of Formula XXXa,

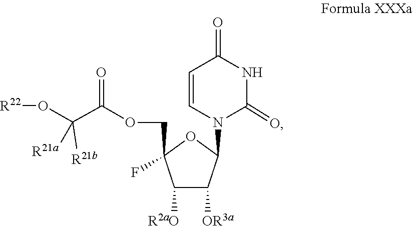

wherein R^{2a} and R^{3a} are as defined above, R²² is C₁-C₆alkyl, and each R^{21a} and R^{21b} is independently selected from a (CH₂)_{q}—(C₁-C₁₂ alkoxy). In certain implementations, R²² is methyl, ethyl, or isopropyl, preferably isopropyl.

In certain embodiments, the disclosure relates to a compound of Formula XXXb,

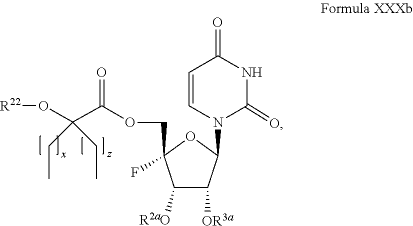

wherein R²² is C₁-C₆alkyl, R^{2a} and R^{3a} are as defined above, each of x and z is independently selected from 0, 1, 2, 3, 4, 5, 6, 7, 8, 9, and 10. In certain implementations, R²² is methyl, ethyl, or isopropyl, preferably isopropyl.

In certain embodiments, the disclosure relates to a compound of Formula XXXc,

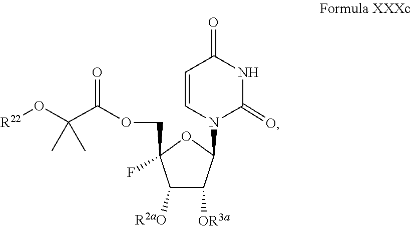

wherein each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid;
wherein said alkyl groups in R^{2a} and R^{3a} can each be optionally independently substituted with one or more, the same or different, R¹⁰; and
wherein R^{2a} and R^{3a} can be optionally covalently bonded, and together with the intermediate atoms, comprise an optionally substituted 5- to 7-membered cycloheteroalkyl;
wherein R²² is selected from hydrogen and C₁-C₂₂ alkyl, for example C₁-C₃ alkyl, C₁-C₆ alkyl, C₁-C₁₂ alkyl, or C₁₂-C₂₂ alkyl.

In certain embodiments, the disclosure relates to a compound of Formula XXXI,

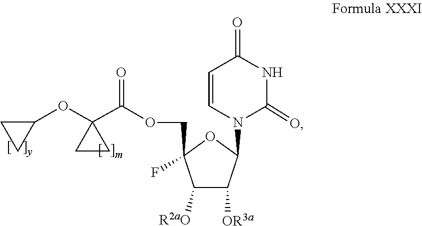

or a pharmaceutically acceptable salt thereof,
wherein each of m and y is independently selected from 1, 2, 3, and 4;
each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid;
wherein said alkyl group in R^{2a} and R^{3a} can each be optionally independently substituted with one or more, the same or different, R¹⁰; and
R^{2a} and R^{3a} can be optionally covalently bonded, and together with the intermediate atoms, comprise an optionally substituted 5- to 7-membered heterocyclyl;

In exemplified embodiments of Formula XXXI, the compound has a structure represented by a formula:

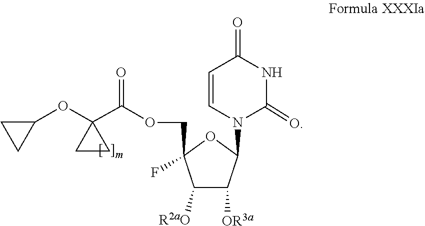

In exemplified embodiments of Formula XXXI, the compound has a structure represented by a formula:

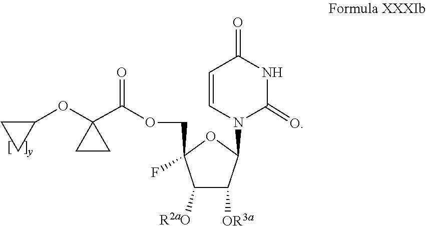

In exemplified embodiments of Formula XXXI, the compound has a structure represented by a formula:

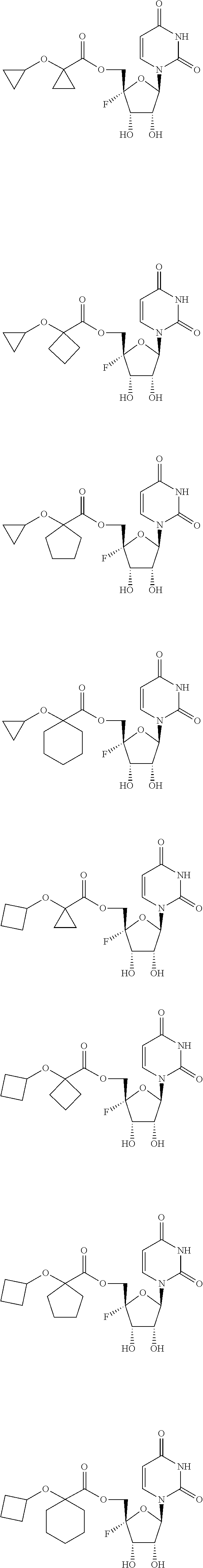

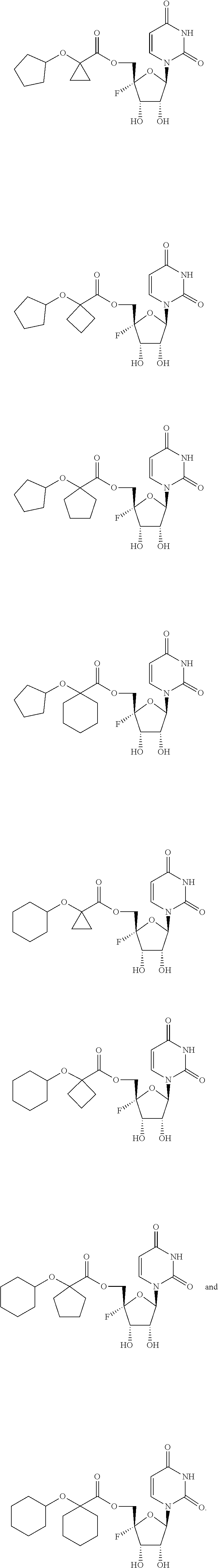

In certain embodiments, the disclosure relates to a compound of Formula XXXII,

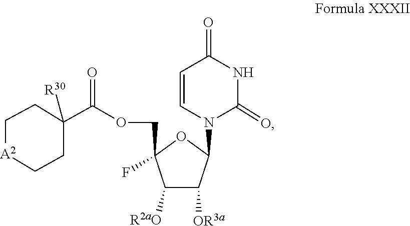

or a pharmaceutically acceptable salt thereof
wherein A² is selected from CH₂, O, S, NC₁-C₃ alkyl, and NH;
wherein each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid;
   wherein R⁴⁰ is in each case independently selected from hydrogen and C₁-C₆alkyl, optionally substituted one more times by R¹⁰;
wherein said alkyl groups in R^{2a} and R^{3a} can each be optionally independently substituted with one or more, the same or different, R¹⁰; and
wherein R^{2a} and R^{3a} can be optionally covalently bonded, and together with the intermediate atoms, comprise an optionally substituted 5- to 7-membered heterocyclyl; and
wherein R³⁰ is selected from hydrogen, deuterium, C₁-C₆ alkyl, and C₁-C₆ alkoxy.

In certain embodiments, the disclosure relates to a compound of Formula XXXIIa,

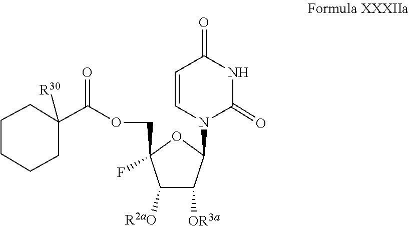

wherein R³⁰ is selected from hydrogen, deuterium, and C₁-C₆ alkyl.

In certain embodiments, the disclosure relates to a compound of Formula XXXIIb,

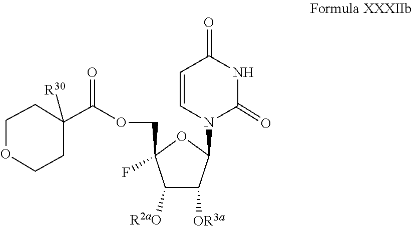

wherein R³⁰ is selected from hydrogen, deuterium, and C₁-C₆ alkyl.

In certain embodiments, the disclosure relates to a compound of Formula XXXIIc,

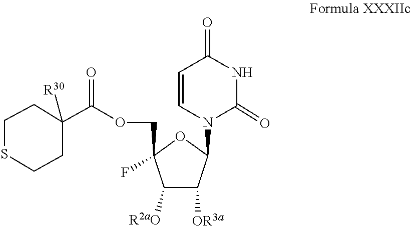

wherein R³⁰ is selected from hydrogen, deuterium, and C₁-C₆ alkyl.

In certain embodiments, the disclosure relates to a compound of Formula XXXIId,

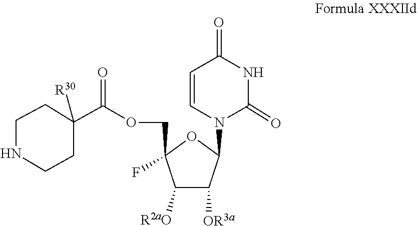

or a pharmaceutically acceptable salt thereof,
wherein R³⁰ is selected from hydrogen, deuterium, and C₁-C₆ alkyl.

In exemplified embodiments of Formula XXXII-XXXIId, R³⁰ is selected from hydrogen and C₁-C₆ alkyl.

In exemplified embodiments of Formula XXXII-XXXIId, R³⁰ is selected from hydrogen, methyl and ethyl.

In exemplified embodiments of Formula XXXII-XXXIId, R³⁰ is selected from hydrogen and methyl.

In exemplified embodiments of Formula XXXII-XXXIId, R³⁰ is hydrogen.

In exemplified embodiments of Formula XXXII-XXXIId, R³⁰ is methyl.

In exemplified embodiments of Formula XXXII-XXXIId, the compound is selected from a structure having a formula:

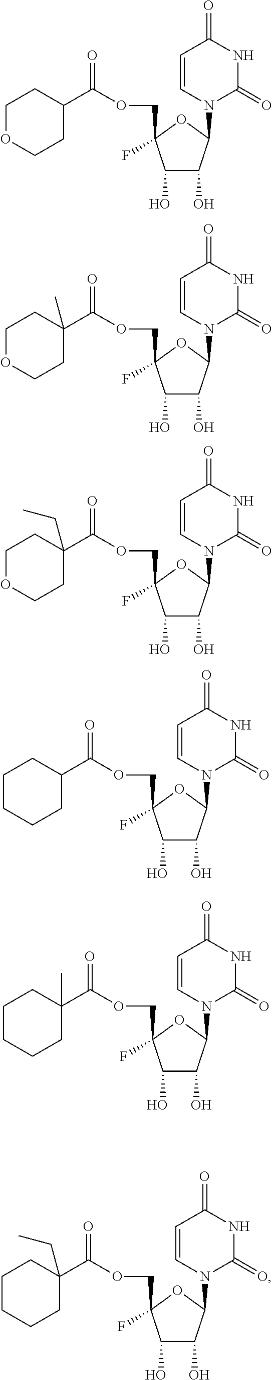

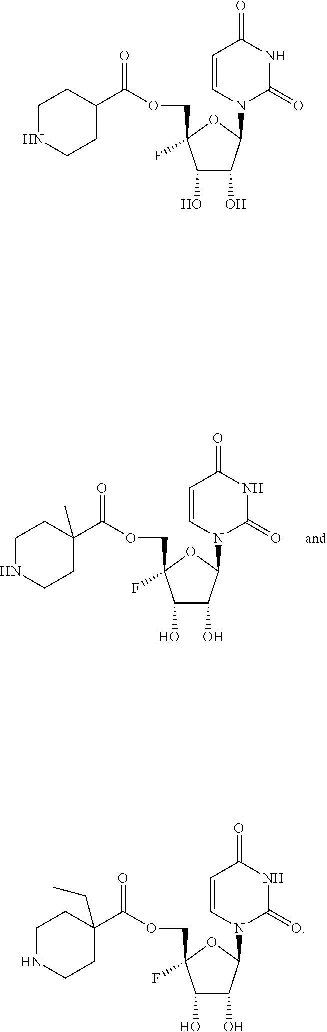 [SMILES: O=C(OC[C@@]1(F)O[C@@H](n2ccc(=O)[nH]c2=O)[C@H](O)[C@@H]1O)C1CCNCC1 ; CC1(C(=O)OC[C@@]2(F)O[C@@H](n3ccc(=O)[nH]c3=O)[C@H](O)[C@@H]2O)CCNCC1 ; CCC1(C(=O)OC[C@@]2(F)O[C@@H](n3ccc(=O)[nH]c3=O)[C@H](O)[C@@H]2O)CCNCC1]

In certain embodiments, the disclosure relates to a compound of Formula XXXIII,

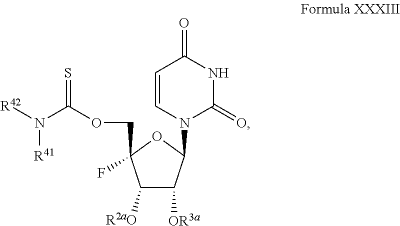

wherein each of R^{2a} and R^{3a} is independently selected from hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid;
wherein said alkyl groups in R^{2a} and R^{3a} can each be optionally independently substituted with one or more, the same or different, R¹⁰; and
wherein R^{2a} and R^{3a} can be optionally covalently bonded, and together with the intermediate atoms, comprise an optionally substituted 5- to 7-membered heterocyclyl; wherein each of R⁴¹ and R⁴² is independently selected from hydrogen and C₁-C₁₂ alkyl, and wherein each of R⁴² and R⁴² can each be optionally independently substituted with one or more, the same or different, R¹⁰;
wherein R¹⁰ is deuterium, hydroxy, azido, thiol, amino, cyano, halogen, alkyl, alkenyl, alkynyl, alkoxy, and alkylamino, wherein R¹⁰ is optionally independently substituted with one or more, the same or different, R^{D}; and
wherein R¹¹ is deuterium, hydroxy, azido, thiol, amino, cyano, halogen, alkyl, alkenyl, alkynyl, alkoxy, alkylamino;
or a pharmaceutically acceptable salt thereof.

In certain embodiments, the disclosure relates to a compound of Formula XXXIV,

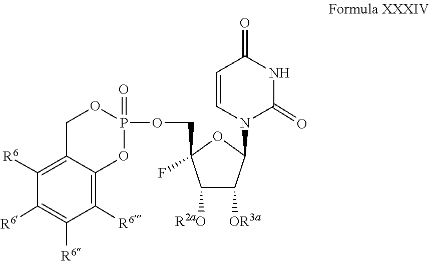

wherein each of R^{2a} and R^{3a} is independently selected from deuterium, hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl and lipid;
wherein R^{2a} and R^{3a} can each be optionally independently substituted with one or more, the same or different, R¹⁰; and
wherein R^{2a} and R^{3a} can be optionally covalently bonded, and together with the intermediate atoms, comprise an optionally substituted 5- to 7-membered heterocyclyl;
wherein R⁶, R^{6′}, R^{6″}, and R^{6′″} are each independently selected from hydrogen, deuterium, hydroxyl, amino, cyano, halogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, or lipid, wherein R⁶, R^{6′}, R^{6″}, and R^{6′″} can each be optionally independently substituted with one or more, the same or different, R¹⁰, provided that at least one of R⁶, R^{6′}, R^{6″}, and R^{6′″} is not hydrogen;
wherein R¹⁰ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, polyethylene glycol, or carbonyl, wherein R¹⁰ is optionally independently substituted with one or more, the same or different, R¹¹;
wherein R¹¹ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, polyethylene glycol, or carbonyl;
   wherein lipid is independently a C₁₁-C₂₂alkyl, C₁₁-C₂₂alkoxy, or aryl substituted with an C₆-C₁₈ alkyl group;
or a pharmaceutically acceptable salt thereof.

In certain embodiments, the disclosure relates to a compound of Formula XXXIVa,

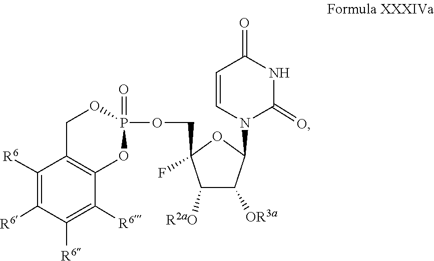

wherein the compound is at least 75 mol %, at least 85 mol %, at least 90 mol %, at least 95 mol %, or at least 99 mol % the depicted stereoisomer at the phosphorous atom,
wherein R⁶, R^{6′}, R^{6″}, and R^{6′″} are each independently selected from hydrogen, deuterium, hydroxyl, amino, cyano, halogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, or lipid, wherein R⁶, R^{6′}, R^{6″}, and R^{6′″} can each be optionally independently substituted with one or more, the same or different, R¹⁰,
wherein R¹⁰ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, polyethylene glycol, or carbonyl, wherein R¹⁰ is optionally independently substituted with one or more, the same or different, R¹¹;
wherein R¹¹ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, polyethylene glycol, or carbonyl;
   wherein lipid is independently a C₁₁-C₂₂alkyl, C₁₁-C₂₂alkoxy, or aryl substituted with an C₆-C₁₈ alkyl group;
or a pharmaceutically acceptable salt thereof.

In certain embodiments, the disclosure relates to a compound of Formula XXXIVa,

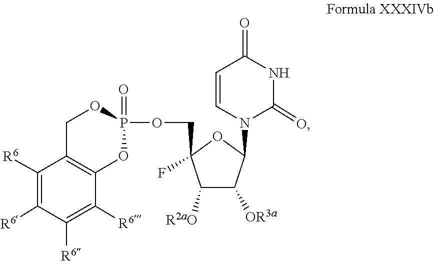

wherein the compound is at least 75 mol %, at least 85 mol %, at least 90 mol %, at least 95 mol %, or at least 99 mol % the depicted stereoisomer at the phosphorous atom
wherein R⁶, R^{6′}, R^{6″}, and R^{6′″} are each independently selected from hydrogen, deuterium, hydroxyl, amino, cyano, halogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, or lipid, wherein R⁶, R^{6′}, R^{6″}, and R^{6′″} can each be optionally independently substituted with one or more, the same or different, R¹⁰;
wherein R¹⁰ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, polyethylene glycol, or carbonyl, wherein R¹⁰ is optionally independently substituted with one or more, the same or different, R¹¹;
wherein R¹¹ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, polyethylene glycol, or carbonyl;
   wherein lipid is independently a C₁₁-C₂₂alkyl, C₁₁-C₂₂alkoxy, or aryl substituted with an C₆-C₁₈ alkyl group;
or a pharmaceutically acceptable salt thereof.

In exemplified embodiments of Formula XXXIV-XXXIVb, one of R⁶, R^{6′}, R^{6″}, and R^{6′″} is not hydrogen.

In exemplified embodiments of Formula XXXIV-XXXIVb, two of R⁶, R^{6′}, R^{6″}, and R^{6′″} are not hydrogen.

In exemplified embodiments of Formula XXXIV-XXXIVb, three of R⁶, R^{6′}, R^{6″}, and R^{6′″} are not hydrogen.

In exemplified embodiments of Formula XXXIV-XXXIVb, R⁶ is not hydrogen.

In exemplified embodiments of Formula XXXIV-XXXIVb, R^{6′} is not hydrogen.

In exemplified embodiments of Formula XXXIV-XXXIVb, R^{6″} is not hydrogen.

In exemplified embodiments of Formula XXXIV-XXXIVb, R^{6′″} is not hydrogen.

In exemplified embodiments of Formula XXXIV-XXXIVb, each of R⁶, R^{6′}, and R^{6″} is hydrogen.

In exemplified embodiments of Formula XXXIV-XXXIVb, each of R^{6′}, R^{6″}, and R^{6′″} is 5 hydrogen.

In exemplified embodiments of Formula XXXIV-XXXIVb, each of R⁶, R^{6′}, and R^{6′″} is hydrogen.

In exemplified embodiments of Formula XXXIV-XXXIVb, each of R⁶, R^{6″}, and R^{6′″} is hydrogen.

In exemplified embodiments of Formula XXXIVa, each of R⁶, R^{6′}, R^{6″}, and R^{6′″} is hydrogen.

In exemplified embodiments of Formula XXXIVb, each of R⁶, R^{6′}, R^{6″}, and R^{6′″} is hydrogen.

In exemplary embodiments, the compound of Formula XXXIV-XXXIVb is selected from:

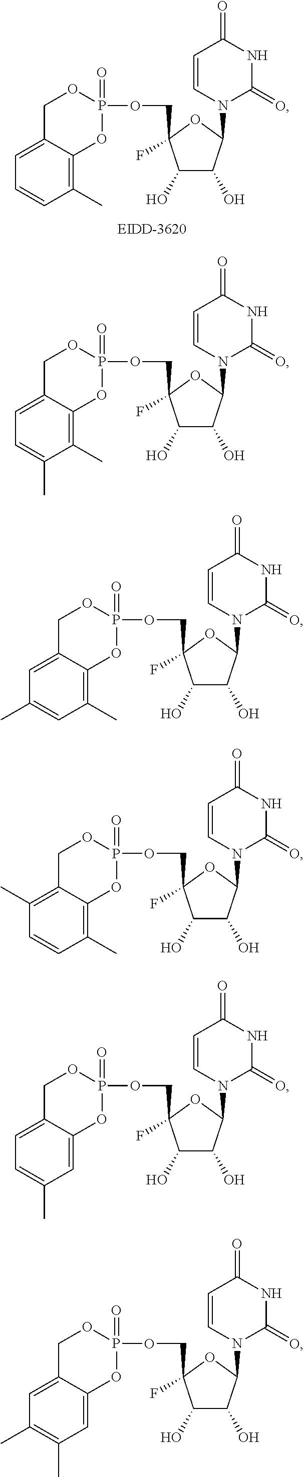

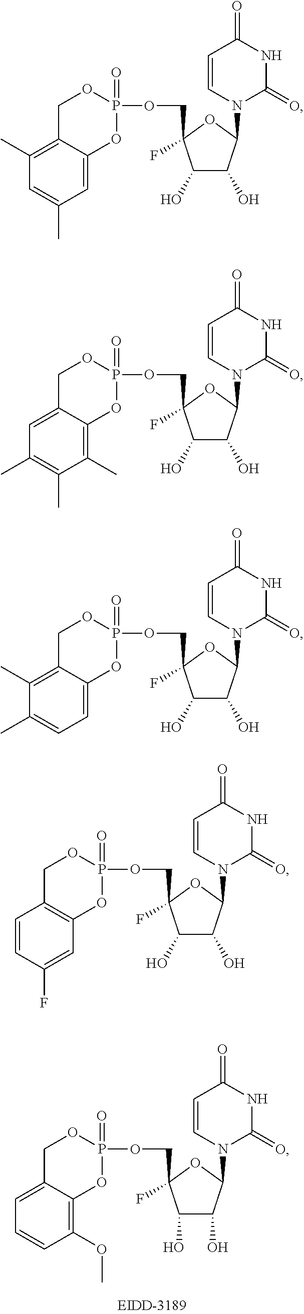 [SMILES: Cc1cc(C)c2c(c1)OP(=O)(OC[C@@]1(F)O[C@@H](n3ccc(=O)[nH]c3=O)[C@H](O)[C@@H]1O)OC2 ; Cc1cc2c(c(C)c1C)OP(=O)(OC[C@@]1(F)O[C@@H](n3ccc(=O)[nH]c3=O)[C@H](O)[C@@H]1O)OC2 ; Cc1ccc2c(c1C)COP(=O)(OC[C@@]1(F)O[C@@H](n3ccc(=O)[nH]c3=O)[C@H](O)[C@@H]1O)O2 ; O=c1ccn([C@@H]2O[C@](F)(COP3(=O)OCc4ccc(F)cc4O3)[C@@H](O)[C@H]2O)c(=O)[nH]1 ; COc1cccc2c1OP(=O)(OC[C@@]1(F)O[C@@H](n3ccc(=O)[nH]c3=O)[C@H](O)[C@@H]1O)OC2]

and combinations of the foregoing.

In exemplary embodiments, the compound is selected from:

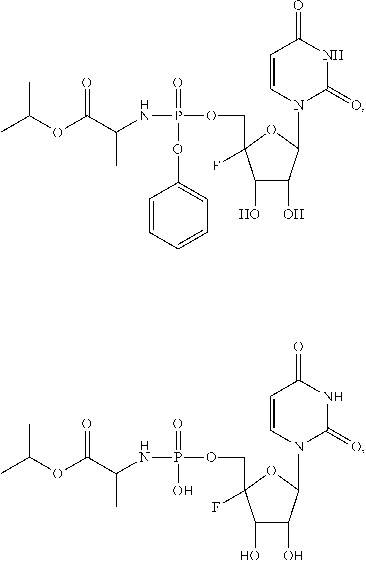 [SMILES: CC(C)OC(=O)C(C)NP(=O)(OCC1(F)OC(n2ccc(=O)[nH]c2=O)C(O)C1O)Oc1ccccc1 ; CC(C)OC(=O)C(C)NP(=O)(O)OCC1(F)OC(n2ccc(=O)[nH]c2=O)C(O)C1O]

and combinations thereof.

In exemplary embodiments, the compound is selected from:

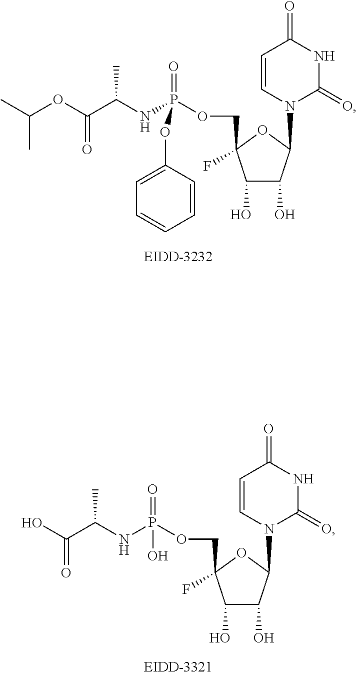 [SMILES: CC(C)OC(=O)[C@H](C)N[P@](=O)(OC[C@@]1(F)O[C@@H](n2ccc(=O)[nH]c2=O)[C@H](O)[C@@H]1O)Oc1ccccc1 ; C[C@H](NP(=O)(O)OC[C@@]1(F)O[C@@H](n2ccc(=O)[nH]c2=O)[C@H](O)[C@@H]1O)C(=O)O]

and combinations thereof.

In exemplary embodiments, the compound is selected from:

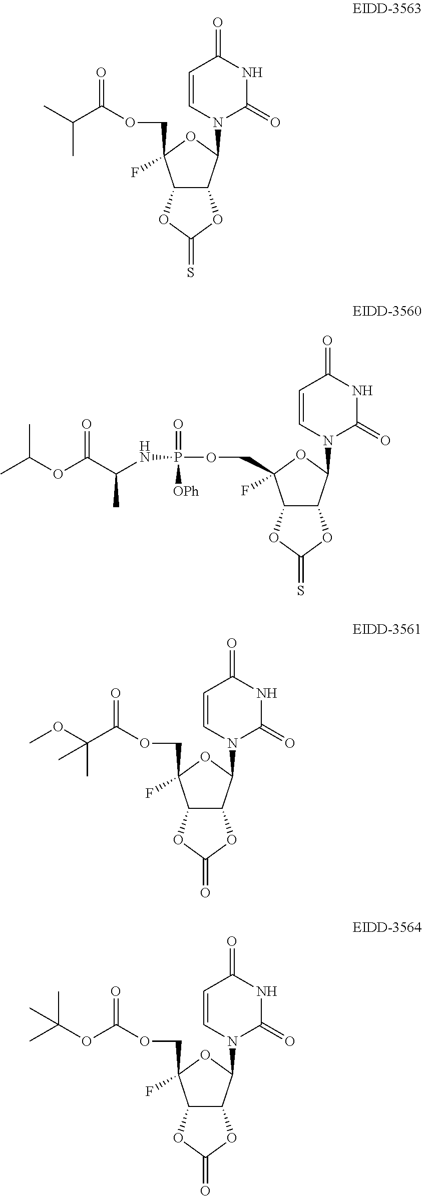 [SMILES: CC(C)C(=O)OC[C@@]1(F)O[C@@H](n2ccc(=O)[nH]c2=O)[C@@H]2OC(=S)O[C@@H]21 ; CC(C)OC(=O)[C@H](C)N[P@](=O)(OC[C@@]1(F)O[C@@H](n2ccc(=O)[nH]c2=O)[C@@H]2OC(=S)O[C@@H]21)Oc1ccccc1 ; COC(C)(C)C(=O)OC[C@@]1(F)O[C@@H](n2ccc(=O)[nH]c2=O)[C@@H]2OC(=O)O[C@@H]21 ; CC(C)(C)OC(=O)OC[C@@]1(F)O[C@@H](n2ccc(=O)[nH]c2=O)[C@@H]2OC(=O)O[C@@H]21]

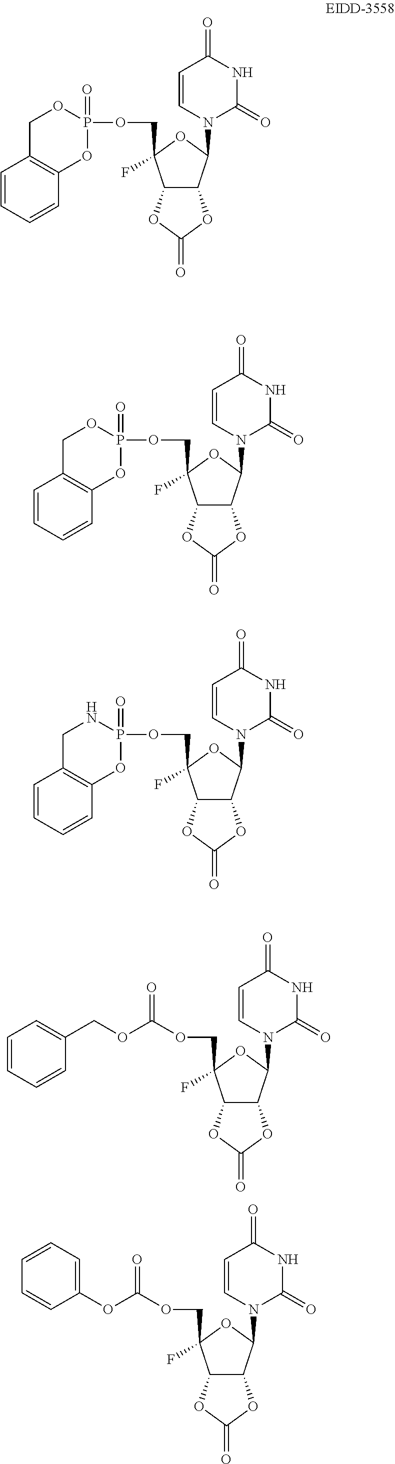

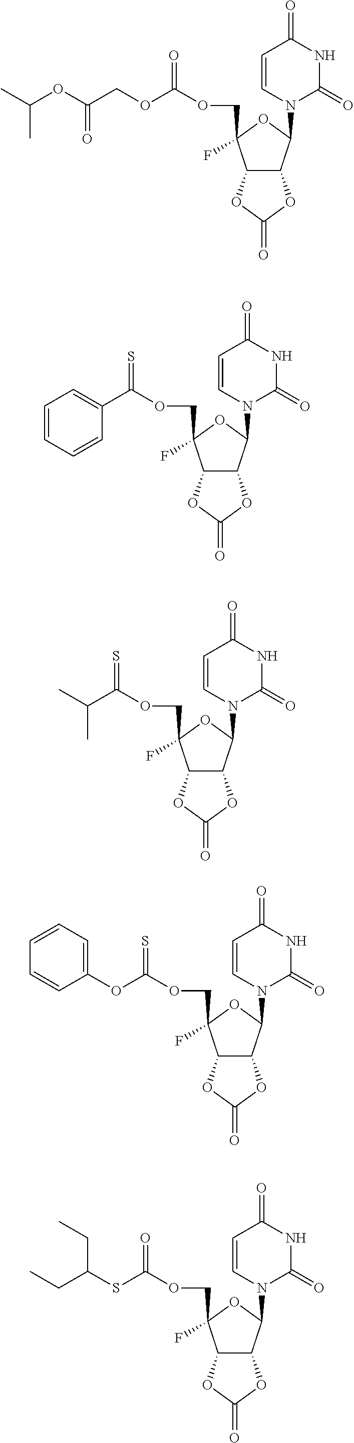 [SMILES: CC(C)OC(=O)COC(=O)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=S)c3ccccc3)[C@H]2O1 ; CC(C)C(=S)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=S)Oc3ccccc3)[C@H]2O1 ; CCC(CC)SC(=O)OC[C@@]1(F)O[C@@H](n2ccc(=O)[nH]c2=O)[C@@H]2OC(=O)O[C@@H]21]

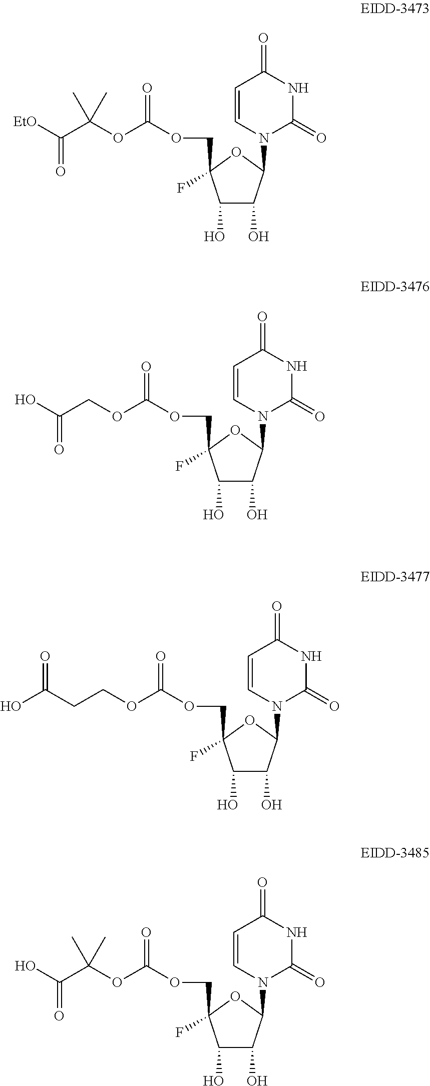 [SMILES: CCOC(=O)C(C)(C)OC(=O)OC[C@@]1(F)O[C@@H](n2ccc(=O)[nH]c2=O)[C@H](O)[C@@H]1O ; O=C(O)COC(=O)OC[C@@]1(F)O[C@@H](n2ccc(=O)[nH]c2=O)[C@H](O)[C@@H]1O ; O=C(O)CCOC(=O)OC[C@@]1(F)O[C@@H](n2ccc(=O)[nH]c2=O)[C@H](O)[C@@H]1O ; CC(C)(OC(=O)OC[C@@]1(F)O[C@@H](n2ccc(=O)[nH]c2=O)[C@H](O)[C@@H]1O)C(=O)O]

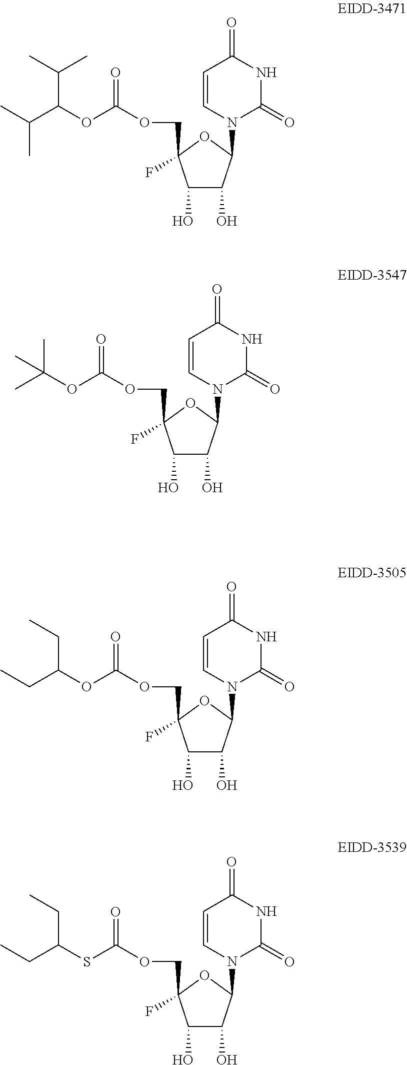 [SMILES: CC(C)C(OC(=O)OC[C@@]1(F)O[C@@H](n2ccc(=O)[nH]c2=O)[C@H](O)[C@@H]1O)C(C)C ; CC(C)(C)OC(=O)OC[C@@]1(F)O[C@@H](n2ccc(=O)[nH]c2=O)[C@H](O)[C@@H]1O ; CCC(CC)OC(=O)OC[C@@]1(F)O[C@@H](n2ccc(=O)[nH]c2=O)[C@H](O)[C@@H]1O ; CCC(CC)SC(=O)OC[C@@]1(F)O[C@@H](n2ccc(=O)[nH]c2=O)[C@H](O)[C@@H]1O]

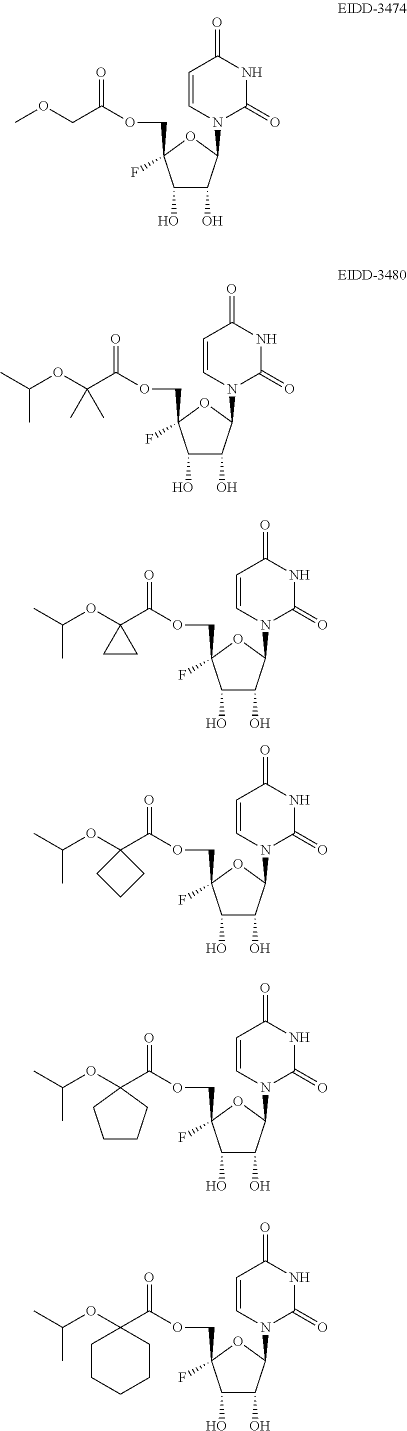

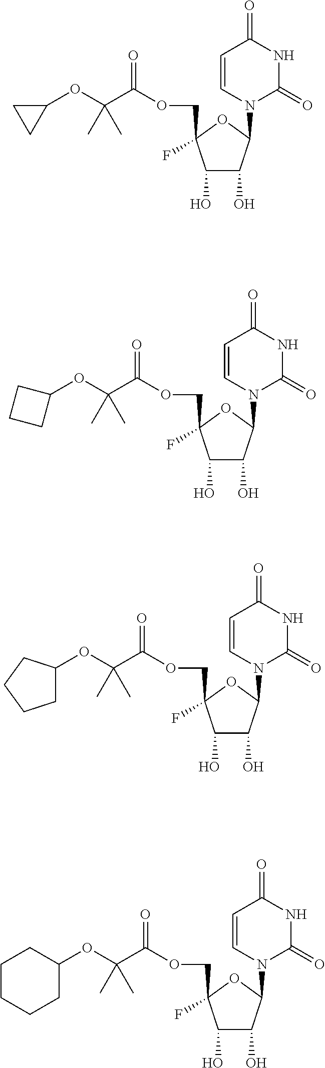 [SMILES: CC(C)(OC1CC1)C(=O)OC[C@@]1(F)O[C@@H](n2ccc(=O)[nH]c2=O)[C@H](O)[C@@H]1O ; CC(C)(OC1CCC1)C(=O)OC[C@@]1(F)O[C@@H](n2ccc(=O)[nH]c2=O)[C@H](O)[C@@H]1O ; CC(C)(OC1CCCC1)C(=O)OC[C@@]1(F)O[C@@H](n2ccc(=O)[nH]c2=O)[C@H](O)[C@@H]1O ; CC(C)(OC1CCCCC1)C(=O)OC[C@@]1(F)O[C@@H](n2ccc(=O)[nH]c2=O)[C@H](O)[C@@H]1O]

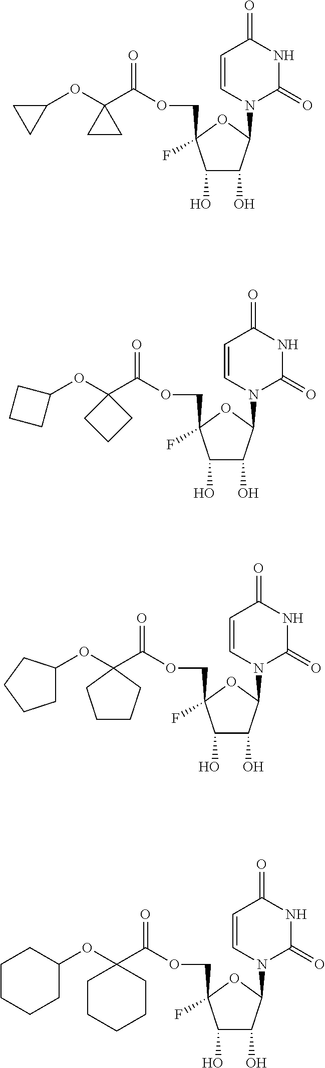 [SMILES: O=C(OC[C@@]1(F)O[C@@H](n2ccc(=O)[nH]c2=O)[C@H](O)[C@@H]1O)C1(OC2CC2)CC1 ; O=C(OC[C@@]1(F)O[C@@H](n2ccc(=O)[nH]c2=O)[C@H](O)[C@@H]1O)C1(OC2CCC2)CCC1 ; O=C(OC[C@@]1(F)O[C@@H](n2ccc(=O)[nH]c2=O)[C@H](O)[C@@H]1O)C1(OC2CCCC2)CCCC1 ; O=C(OC[C@@]1(F)O[C@@H](n2ccc(=O)[nH]c2=O)[C@H](O)[C@@H]1O)C1(OC2CCCCC2)CCCCC1]

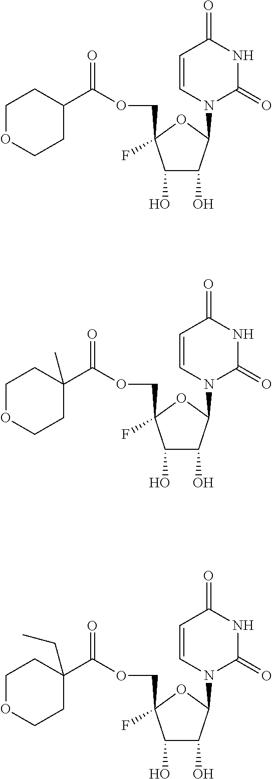 [SMILES: O=C(OC[C@@]1(F)O[C@@H](n2ccc(=O)[nH]c2=O)[C@H](O)[C@@H]1O)C1CCOCC1 ; CC1(C(=O)OC[C@@]2(F)O[C@@H](n3ccc(=O)[nH]c3=O)[C@H](O)[C@@H]2O)CCOCC1 ; CCC1(C(=O)OC[C@@]2(F)O[C@@H](n3ccc(=O)[nH]c3=O)[C@H](O)[C@@H]2O)CCOCC1]

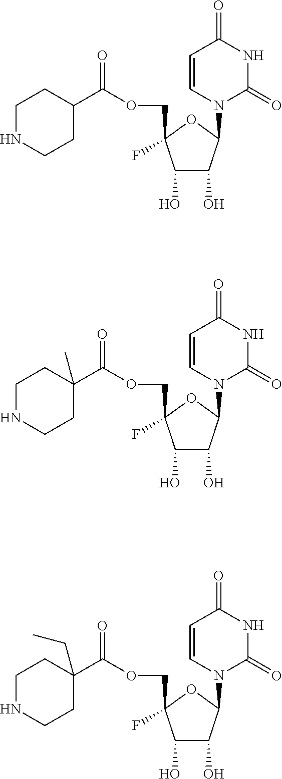 [SMILES: O=C(OC[C@@]1(F)O[C@@H](n2ccc(=O)[nH]c2=O)[C@H](O)[C@@H]1O)C1CCNCC1 ; CC1(C(=O)OC[C@@]2(F)O[C@@H](n3ccc(=O)[nH]c3=O)[C@H](O)[C@@H]2O)CCNCC1 ; CCC1(C(=O)OC[C@@]2(F)O[C@@H](n3ccc(=O)[nH]c3=O)[C@H](O)[C@@H]2O)CCNCC1]

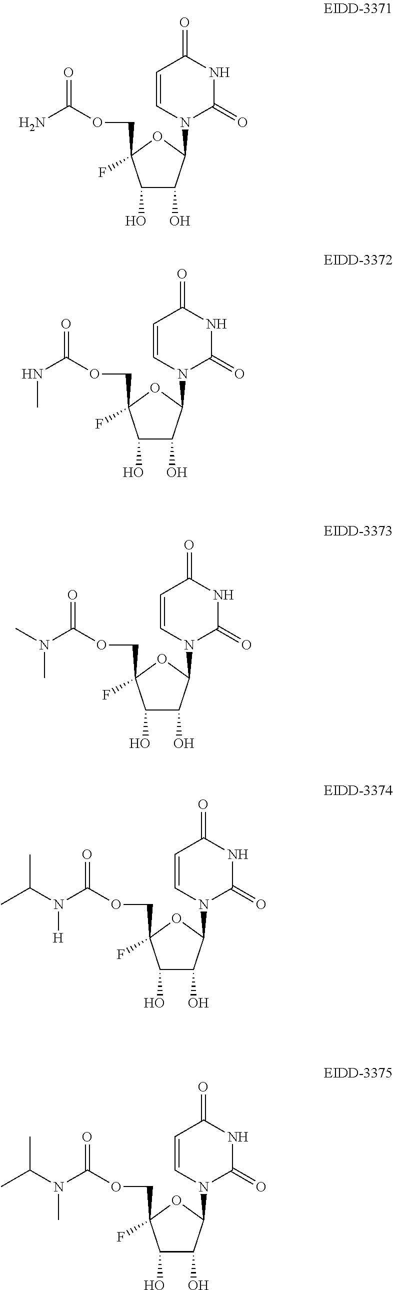 [SMILES: NC(=O)OC[C@@]1(F)O[C@@H](n2ccc(=O)[nH]c2=O)[C@H](O)[C@@H]1O ; CNC(=O)OC[C@@]1(F)O[C@@H](n2ccc(=O)[nH]c2=O)[C@H](O)[C@@H]1O ; CN(C)C(=O)OC[C@@]1(F)O[C@@H](n2ccc(=O)[nH]c2=O)[C@H](O)[C@@H]1O ; CC(C)NC(=O)OC[C@@]1(F)O[C@@H](n2ccc(=O)[nH]c2=O)[C@H](O)[C@@H]1O ; CC(C)N(C)C(=O)OC[C@@]1(F)O[C@@H](n2ccc(=O)[nH]c2=O)[C@H](O)[C@@H]1O]

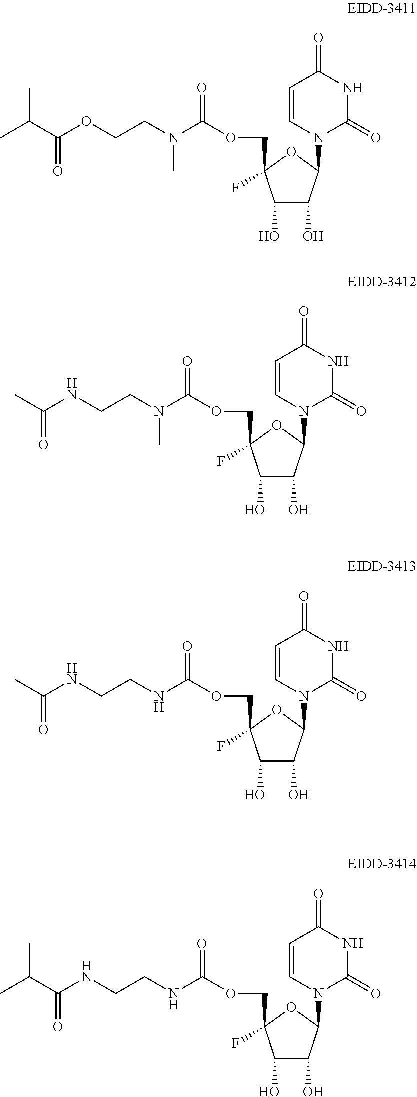 [SMILES: CC(C)C(=O)OCCN(C)C(=O)OC[C@@]1(F)O[C@@H](n2ccc(=O)[nH]c2=O)[C@H](O)[C@@H]1O ; CC(=O)NCCN(C)C(=O)OC[C@@]1(F)O[C@@H](n2ccc(=O)[nH]c2=O)[C@H](O)[C@@H]1O ; CC(=O)NCCNC(=O)OC[C@@]1(F)O[C@@H](n2ccc(=O)[nH]c2=O)[C@H](O)[C@@H]1O ; CC(C)C(=O)NCCNC(=O)OC[C@@]1(F)O[C@@H](n2ccc(=O)[nH]c2=O)[C@H](O)[C@@H]1O]

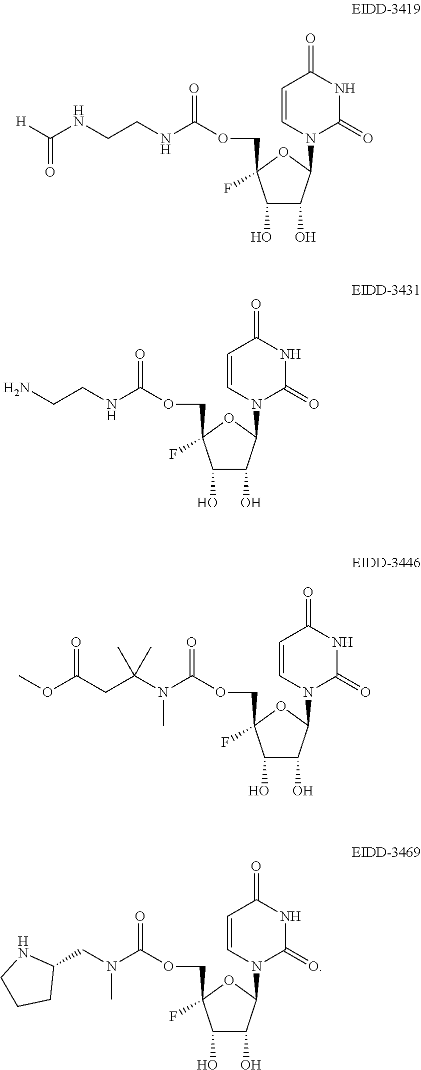 [SMILES: O=CNCCNC(=O)OC[C@@]1(F)O[C@@H](n2ccc(=O)[nH]c2=O)[C@H](O)[C@@H]1O ; NCCNC(=O)OC[C@@]1(F)O[C@@H](n2ccc(=O)[nH]c2=O)[C@H](O)[C@@H]1O ; COC(=O)CC(C)(C)N(C)C(=O)OC[C@@]1(F)O[C@@H](n2ccc(=O)[nH]c2=O)[C@H](O)[C@@H]1O ; CN(C[C@@H]1CCCN1)C(=O)OC[C@@]1(F)O[C@@H](n2ccc(=O)[nH]c2=O)[C@H](O)[C@@H]1O]

In exemplary embodiments, the compound is selected from:

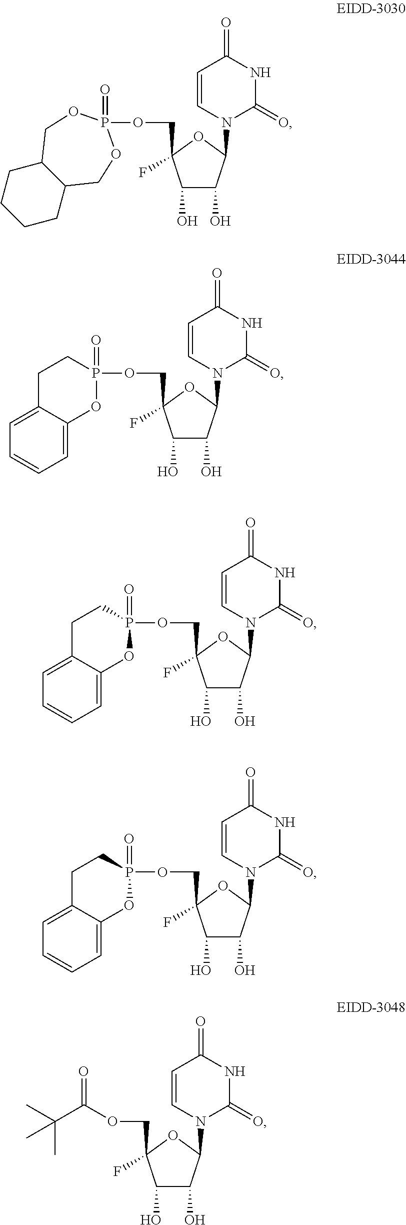 [SMILES: O=c1ccn([C@@H]2O[C@](F)(COP3(=O)OCC4CCCCC4CO3)[C@@H](O)[C@H]2O)c(=O)[nH]1 ; O=c1ccn([C@@H]2O[C@](F)(COP3(=O)CCc4ccccc4O3)[C@@H](O)[C@H]2O)c(=O)[nH]1 ; O=c1ccn([C@@H]2O[C@](F)(CO[P@@]3(=O)CCc4ccccc4O3)[C@@H](O)[C@H]2O)c(=O)[nH]1 ; O=c1ccn([C@@H]2O[C@](F)(CO[P@]3(=O)CCc4ccccc4O3)[C@@H](O)[C@H]2O)c(=O)[nH]1 ; CC(C)(C)C(=O)OC[C@@]1(F)O[C@@H](n2ccc(=O)[nH]c2=O)[C@H](O)[C@@H]1O]

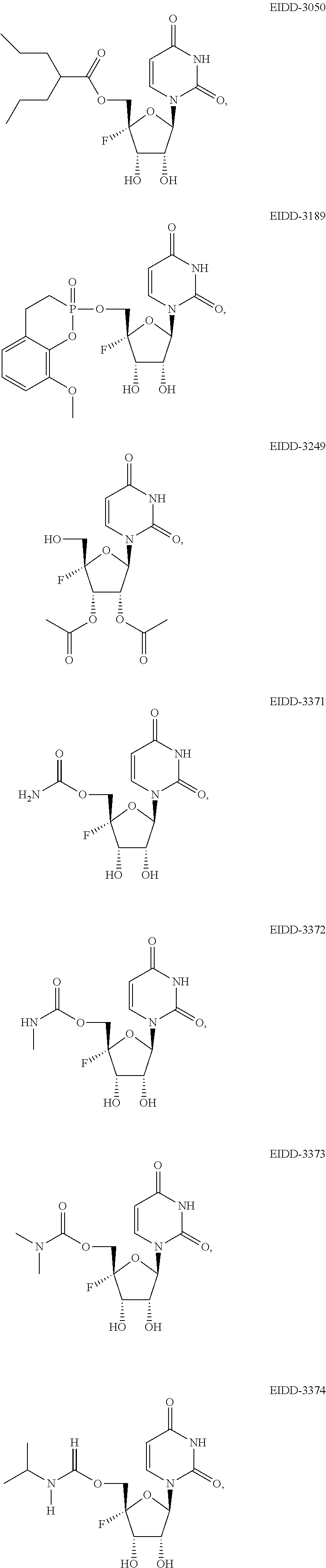

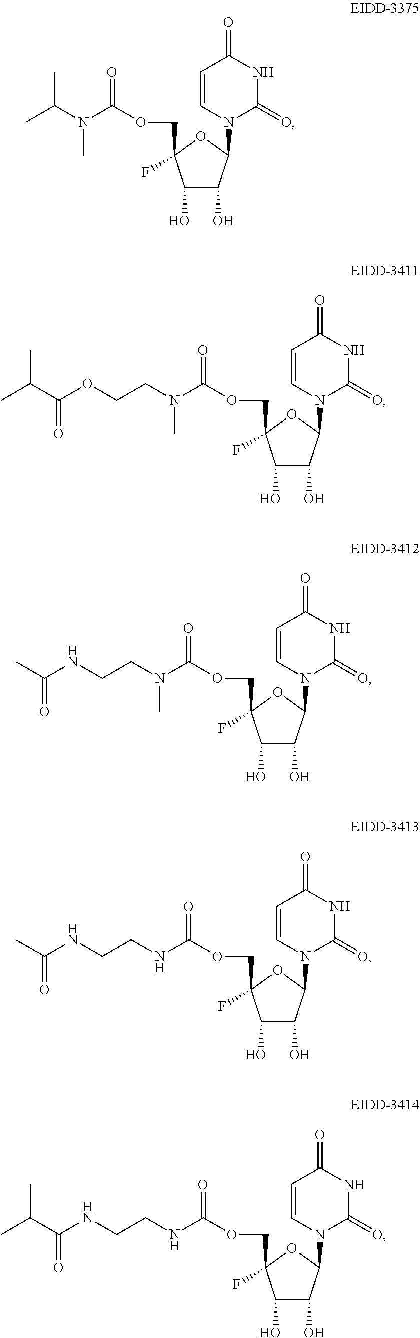 [SMILES: CC(C)N(C)C(=O)OC[C@@]1(F)O[C@@H](n2ccc(=O)[nH]c2=O)[C@H](O)[C@@H]1O ; CC(C)C(=O)OCCN(C)C(=O)OC[C@@]1(F)O[C@@H](n2ccc(=O)[nH]c2=O)[C@H](O)[C@@H]1O ; CC(=O)NCCN(C)C(=O)OC[C@@]1(F)O[C@@H](n2ccc(=O)[nH]c2=O)[C@H](O)[C@@H]1O ; CC(=O)NCCNC(=O)OC[C@@]1(F)O[C@@H](n2ccc(=O)[nH]c2=O)[C@H](O)[C@@H]1O ; CC(C)C(=O)NCCNC(=O)OC[C@@]1(F)O[C@@H](n2ccc(=O)[nH]c2=O)[C@H](O)[C@@H]1O]

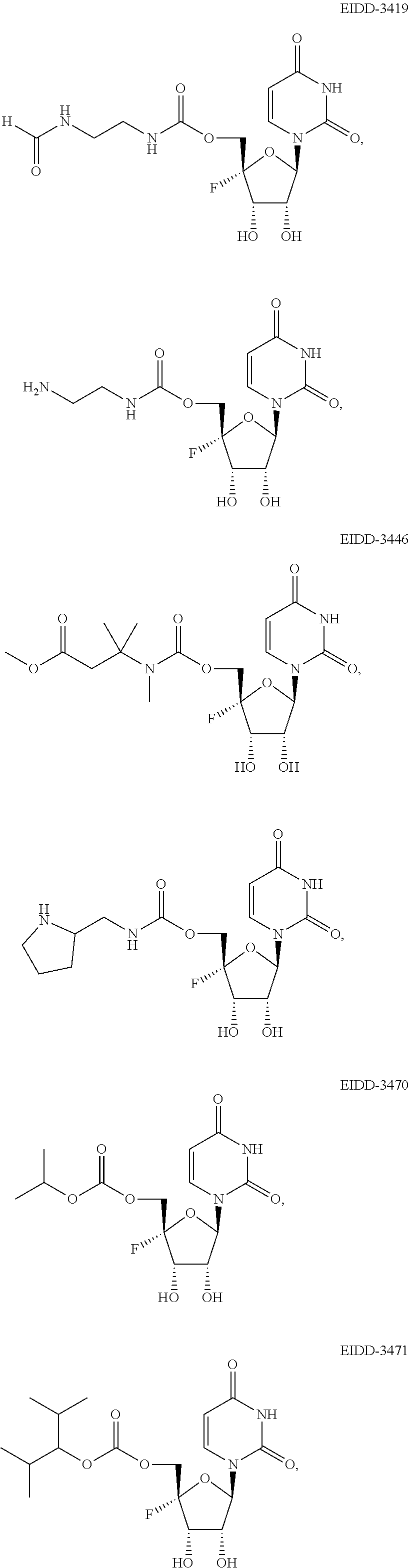 [SMILES: O=CNCCNC(=O)OC[C@@]1(F)O[C@@H](n2ccc(=O)[nH]c2=O)[C@H](O)[C@@H]1O ; NCCNC(=O)OC[C@@]1(F)O[C@@H](n2ccc(=O)[nH]c2=O)[C@H](O)[C@@H]1O ; COC(=O)CC(C)(C)N(C)C(=O)OC[C@@]1(F)O[C@@H](n2ccc(=O)[nH]c2=O)[C@H](O)[C@@H]1O ; O=C(NCC1CCCN1)OC[C@@]1(F)O[C@@H](n2ccc(=O)[nH]c2=O)[C@H](O)[C@@H]1O ; CC(C)OC(=O)OC[C@@]1(F)O[C@@H](n2ccc(=O)[nH]c2=O)[C@H](O)[C@@H]1O ; CC(C)C(OC(=O)OC[C@@]1(F)O[C@@H](n2ccc(=O)[nH]c2=O)[C@H](O)[C@@H]1O)C(C)C]

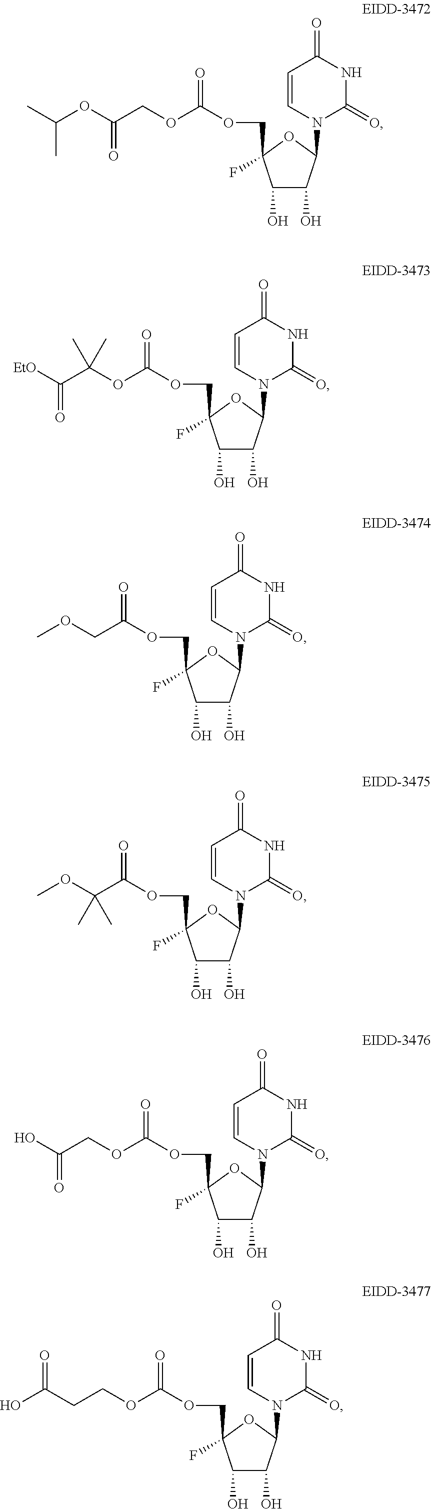 [SMILES: CC(C)OC(=O)COC(=O)OC[C@@]1(F)O[C@@H](n2ccc(=O)[nH]c2=O)[C@H](O)[C@@H]1O ; CCOC(=O)C(C)(C)OC(=O)OC[C@@]1(F)O[C@@H](n2ccc(=O)[nH]c2=O)[C@H](O)[C@@H]1O ; COCC(=O)OC[C@@]1(F)O[C@@H](n2ccc(=O)[nH]c2=O)[C@H](O)[C@@H]1O ; COC(C)(C)C(=O)OC[C@@]1(F)O[C@@H](n2ccc(=O)[nH]c2=O)[C@H](O)[C@@H]1O ; O=C(O)COC(=O)OC[C@@]1(F)O[C@@H](n2ccc(=O)[nH]c2=O)[C@H](O)[C@@H]1O ; O=C(O)CCOC(=O)OC[C@@]1(F)O[C@@H](n2ccc(=O)[nH]c2=O)[C@H](O)[C@@H]1O]

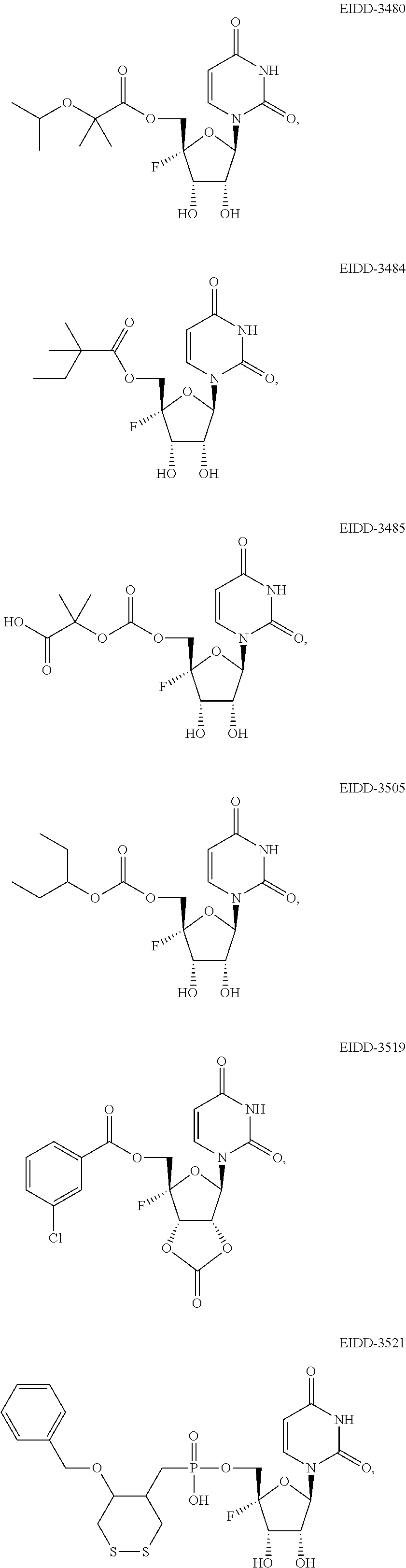 [SMILES: CC(C)OC(C)(C)C(=O)OC[C@@]1(F)O[C@@H](n2ccc(=O)[nH]c2=O)[C@H](O)[C@@H]1O ; CCC(C)(C)C(=O)OC[C@@]1(F)O[C@@H](n2ccc(=O)[nH]c2=O)[C@H](O)[C@@H]1O ; CC(C)(OC(=O)OC[C@@]1(F)O[C@@H](n2ccc(=O)[nH]c2=O)[C@H](O)[C@@H]1O)C(=O)O ; CCC(CC)OC(=O)OC[C@@]1(F)O[C@@H](n2ccc(=O)[nH]c2=O)[C@H](O)[C@@H]1O ; O=C1O[C@H]2[C@H](n3ccc(=O)[nH]c3=O)O[C@](F)(COC(=O)c3cccc(Cl)c3)[C@H]2O1 ; O=c1ccn([C@@H]2O[C@](F)(COP(=O)(O)CC3CSSCC3OCc3ccccc3)[C@@H](O)[C@H]2O)c(=O)[nH]1]

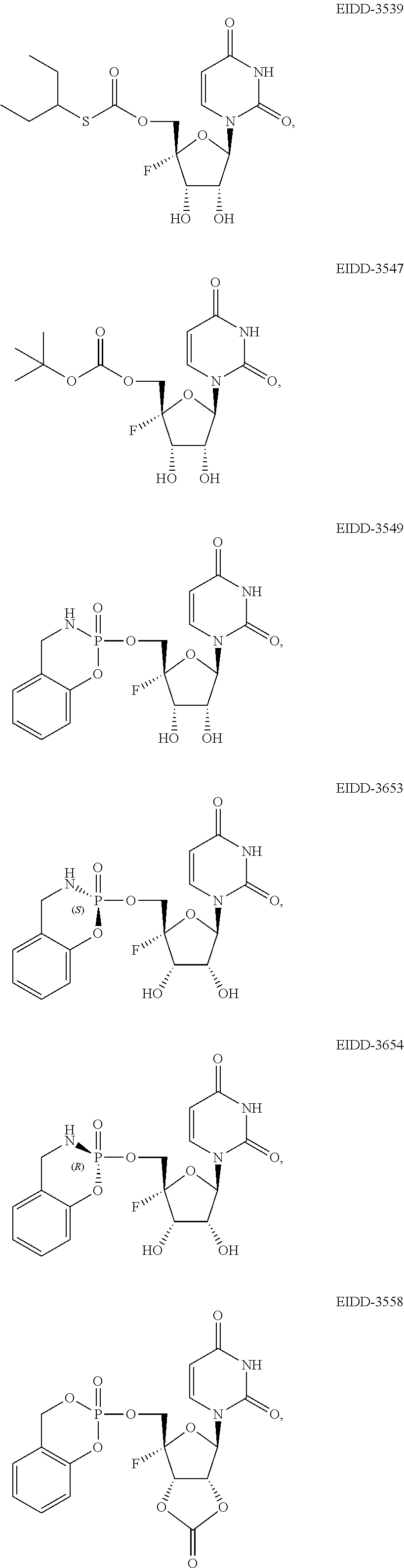

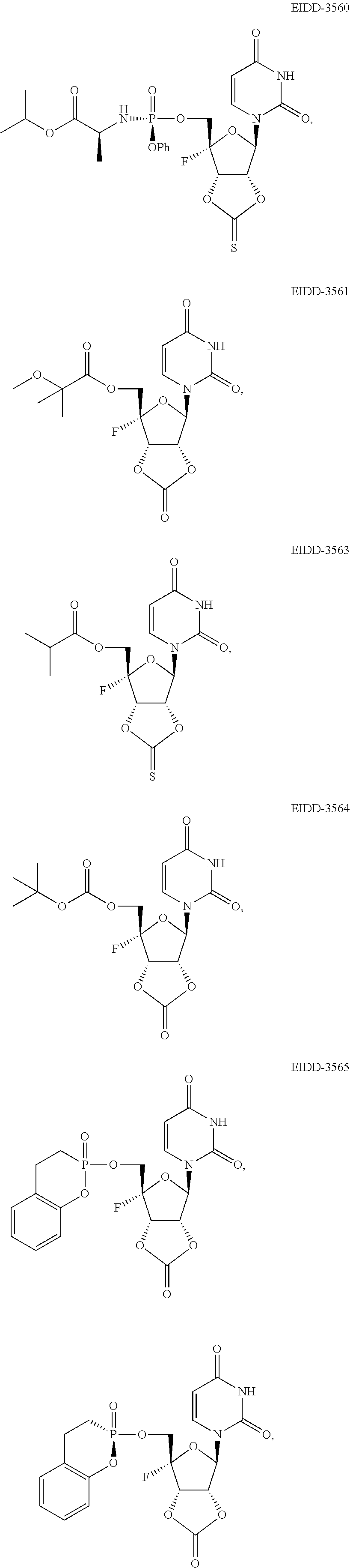 [SMILES: CC(C)OC(=O)[C@H](C)N[P@](=O)(OC[C@@]1(F)O[C@@H](n2ccc(=O)[nH]c2=O)[C@@H]2OC(=S)O[C@@H]21)Oc1ccccc1 ; COC(C)(C)C(=O)OC[C@@]1(F)O[C@@H](n2ccc(=O)[nH]c2=O)[C@@H]2OC(=O)O[C@@H]21 ; CC(C)C(=O)OC[C@@]1(F)O[C@@H](n2ccc(=O)[nH]c2=O)[C@@H]2OC(=S)O[C@@H]21 ; CC(C)(C)OC(=O)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COP3(=O)CCc4ccccc4O3)[C@H]2O1 ; O=C1O[C@H]2[C@H](n3ccc(=O)[nH]c3=O)O[C@](F)(CO[P@@]3(=O)CCc4ccccc4O3)[C@H]2O1]

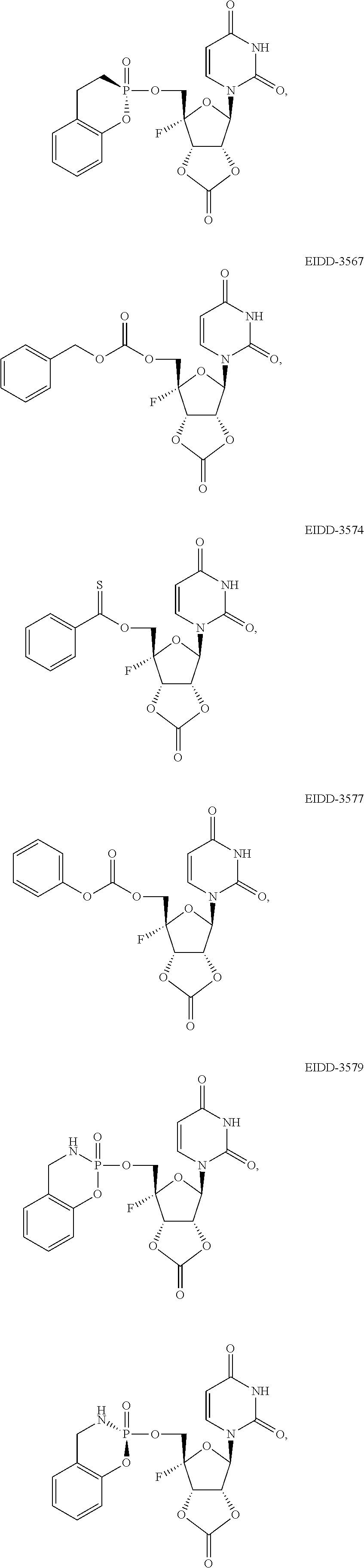 [SMILES: O=C1O[C@H]2[C@H](n3ccc(=O)[nH]c3=O)O[C@](F)(CO[P@]3(=O)CCc4ccccc4O3)[C@H]2O1 ; O=C(OCc1ccccc1)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=S)c3ccccc3)[C@H]2O1 ; O=C(OC[C@@]1(F)O[C@@H](n2ccc(=O)[nH]c2=O)[C@@H]2OC(=O)O[C@@H]21)Oc1ccccc1 ; O=C1O[C@H]2[C@H](n3ccc(=O)[nH]c3=O)O[C@](F)(COP3(=O)NCc4ccccc4O3)[C@H]2O1 ; O=C1O[C@H]2[C@H](n3ccc(=O)[nH]c3=O)O[C@](F)(CO[P@@]3(=O)NCc4ccccc4O3)[C@H]2O1]

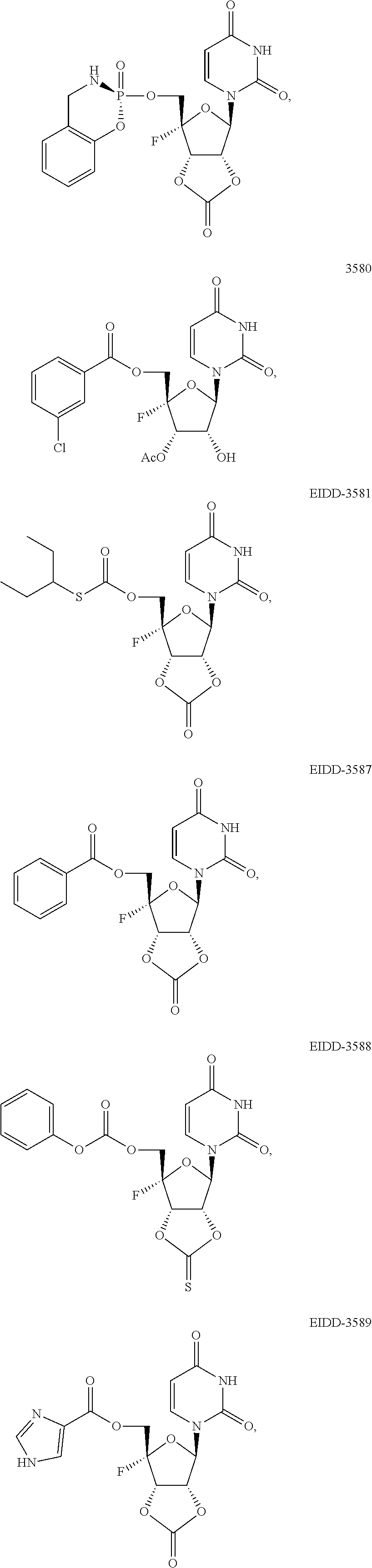 [SMILES: O=C1O[C@H]2[C@H](n3ccc(=O)[nH]c3=O)O[C@](F)(CO[P@]3(=O)NCc4ccccc4O3)[C@H]2O1 ; CC(=O)O[C@H]1[C@@H](O)[C@H](n2ccc(=O)[nH]c2=O)O[C@]1(F)COC(=O)c1cccc(Cl)c1 ; CCC(CC)SC(=O)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=O)c3ccccc3)[C@H]2O1 ; O=C(OC[C@@]1(F)O[C@@H](n2ccc(=O)[nH]c2=O)[C@@H]2OC(=S)O[C@@H]21)Oc1ccccc1 ; O=C1O[C@H]2[C@H](n3ccc(=O)[nH]c3=O)O[C@](F)(COC(=O)c3c[nH]cn3)[C@H]2O1]

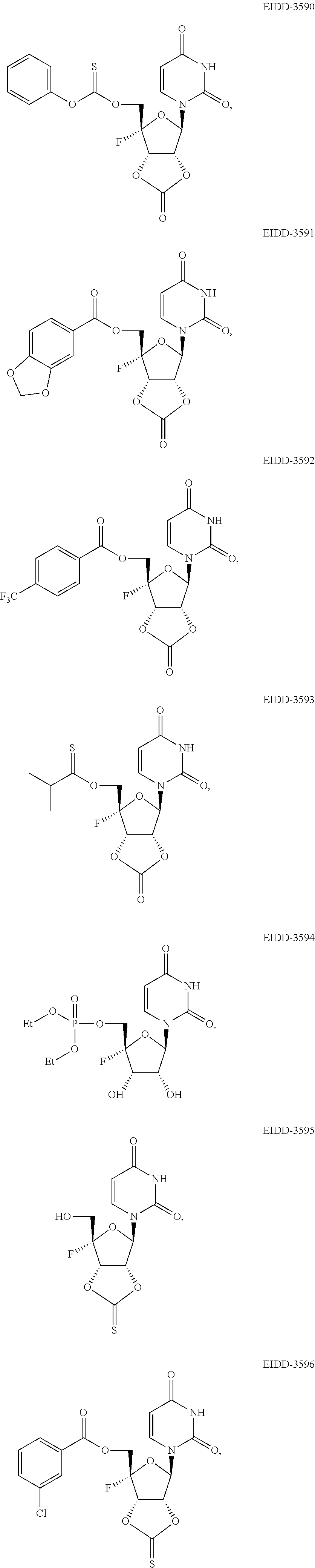

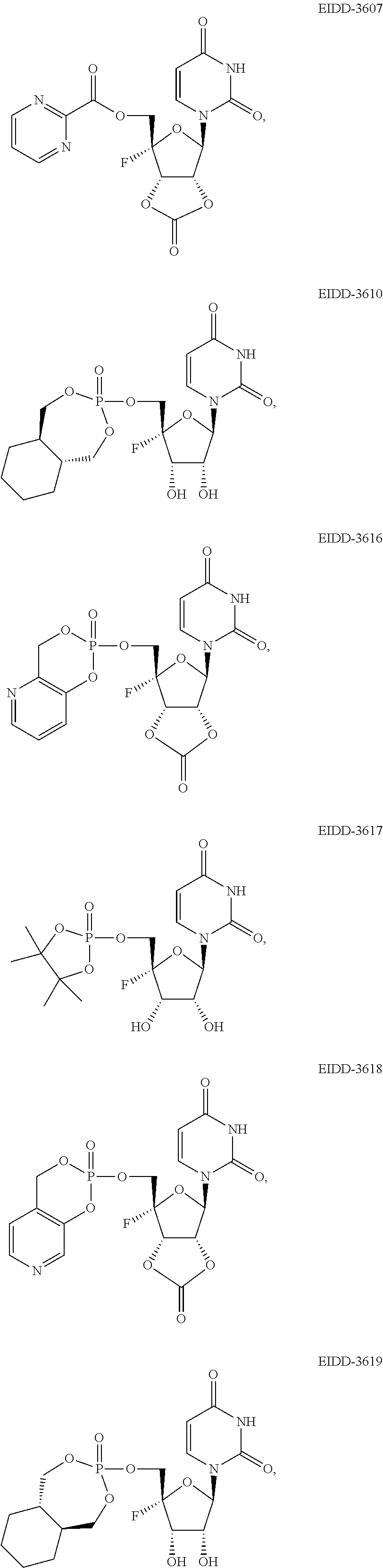 [SMILES: O=C1O[C@H]2[C@H](n3ccc(=O)[nH]c3=O)O[C@](F)(COC(=O)c3ncccn3)[C@H]2O1 ; O=c1ccn([C@@H]2O[C@](F)(COP3(=O)OC[C@@H]4CCCC[C@H]4CO3)[C@@H](O)[C@H]2O)c(=O)[nH]1 ; O=C1O[C@H]2[C@H](n3ccc(=O)[nH]c3=O)O[C@](F)(COP3(=O)OCc4ncccc4O3)[C@H]2O1 ; CC1(C)OP(=O)(OC[C@@]2(F)O[C@@H](n3ccc(=O)[nH]c3=O)[C@H](O)[C@@H]2O)OC1(C)C ; O=C1O[C@H]2[C@H](n3ccc(=O)[nH]c3=O)O[C@](F)(COP3(=O)OCc4ccncc4O3)[C@H]2O1 ; O=c1ccn([C@@H]2O[C@](F)(COP3(=O)OC[C@H]4CCCC[C@@H]4CO3)[C@@H](O)[C@H]2O)c(=O)[nH]1]

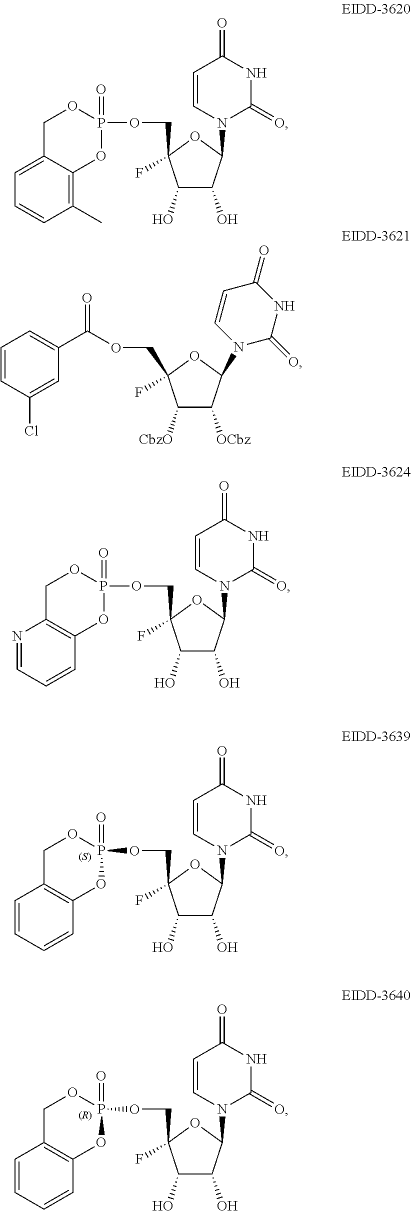

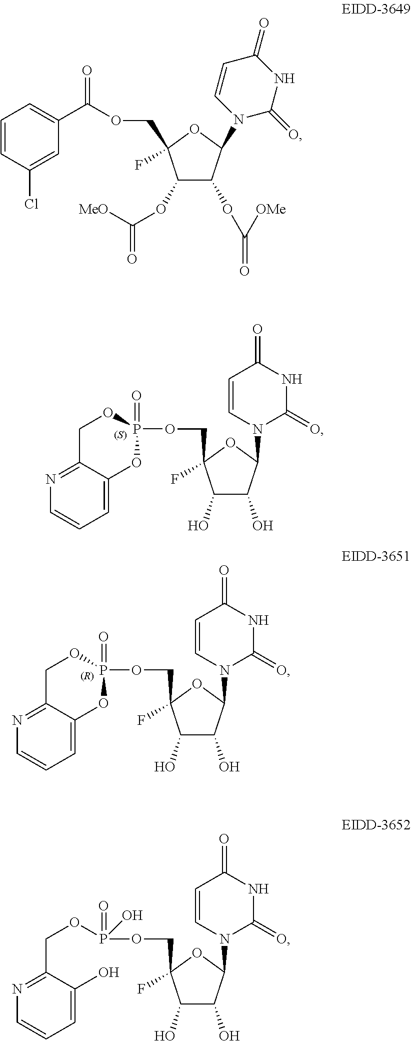

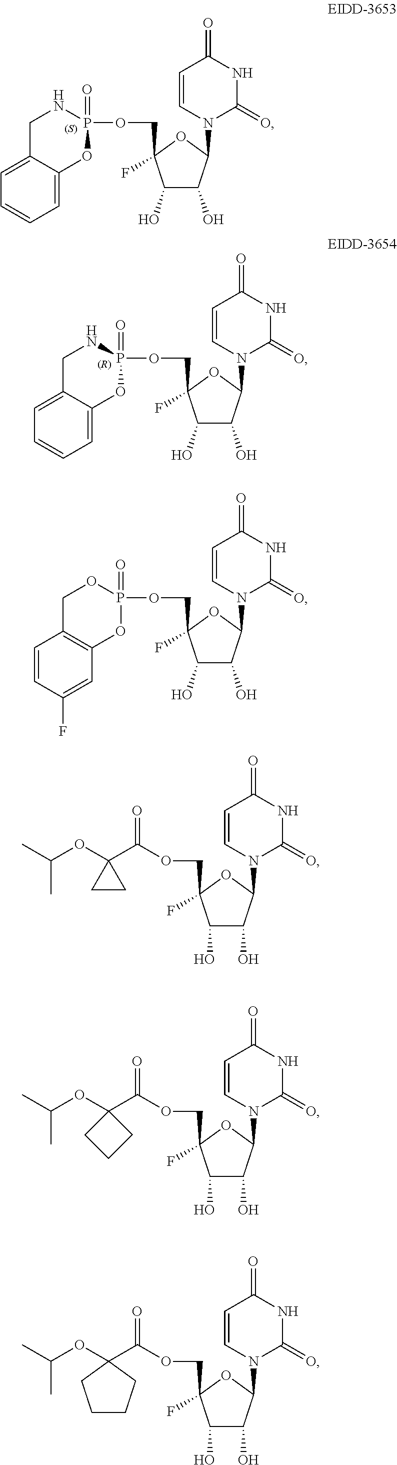

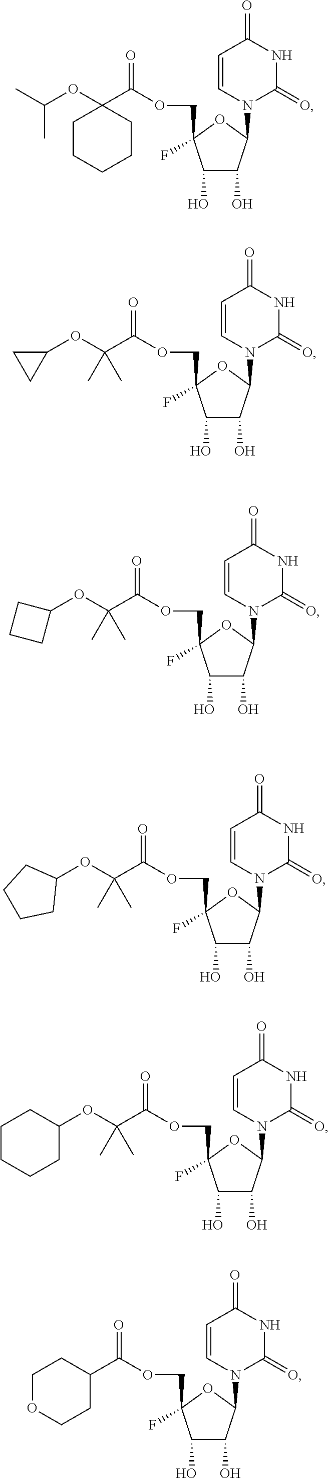 [SMILES: CC(C)OC1(C(=O)OC[C@@]2(F)O[C@@H](n3ccc(=O)[nH]c3=O)[C@H](O)[C@@H]2O)CCCCC1 ; CC(C)(OC1CC1)C(=O)OC[C@@]1(F)O[C@@H](n2ccc(=O)[nH]c2=O)[C@H](O)[C@@H]1O ; CC(C)(OC1CCC1)C(=O)OC[C@@]1(F)O[C@@H](n2ccc(=O)[nH]c2=O)[C@H](O)[C@@H]1O ; CC(C)(OC1CCCC1)C(=O)OC[C@@]1(F)O[C@@H](n2ccc(=O)[nH]c2=O)[C@H](O)[C@@H]1O ; CC(C)(OC1CCCCC1)C(=O)OC[C@@]1(F)O[C@@H](n2ccc(=O)[nH]c2=O)[C@H](O)[C@@H]1O ; O=C(OC[C@@]1(F)O[C@@H](n2ccc(=O)[nH]c2=O)[C@H](O)[C@@H]1O)C1CCOCC1]

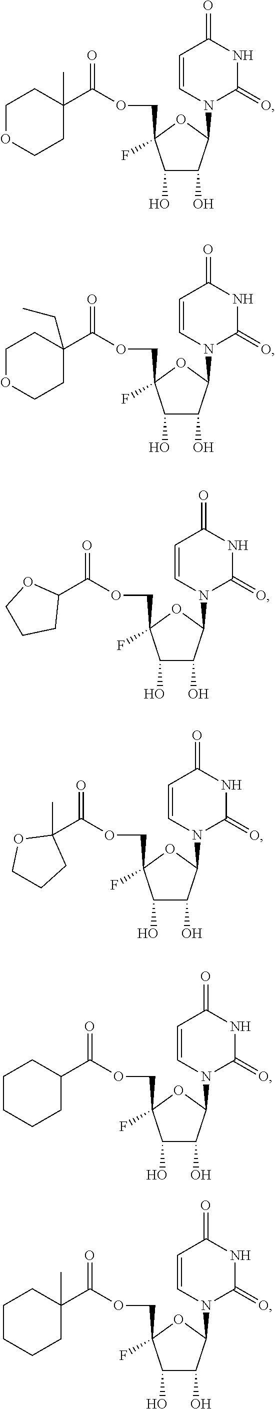

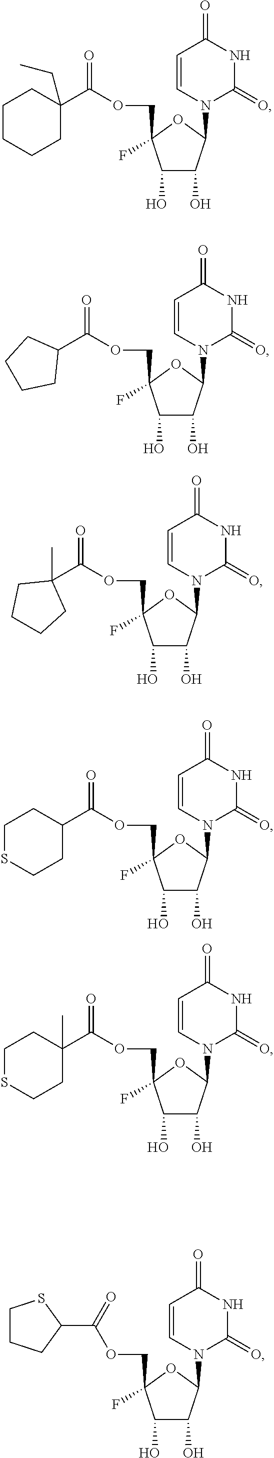

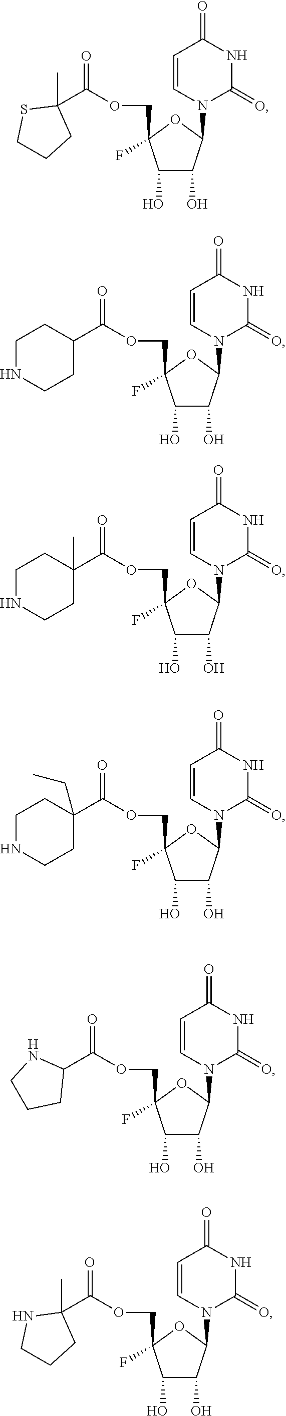

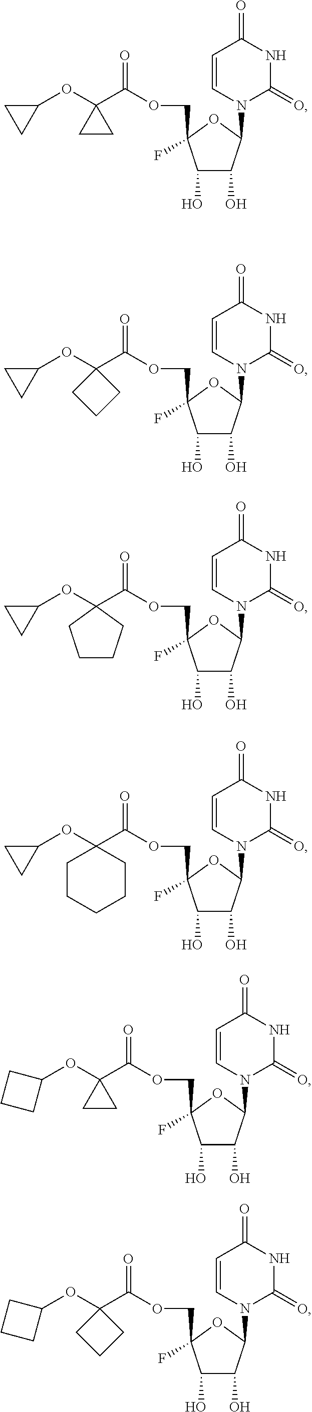

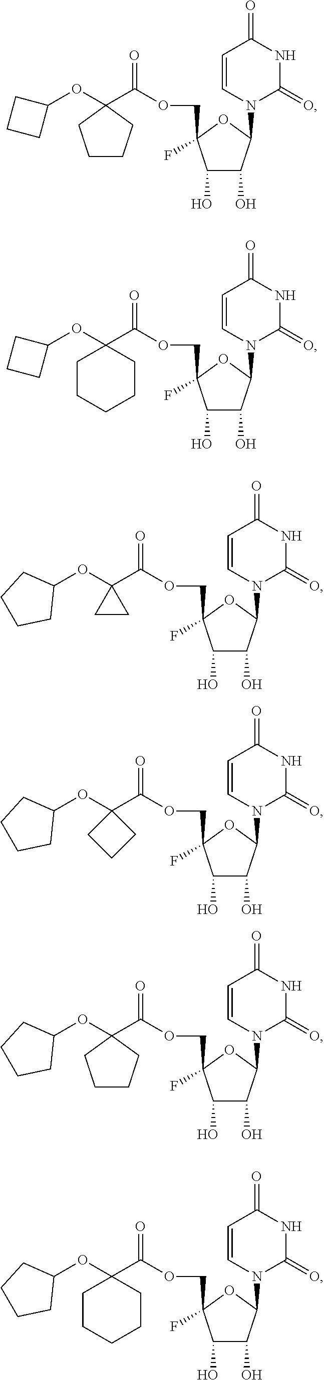

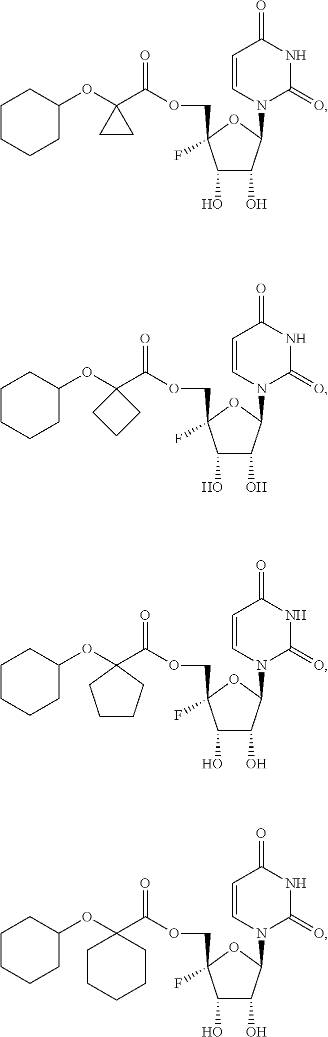 [SMILES: O=C(OC[C@@]1(F)O[C@@H](n2ccc(=O)[nH]c2=O)[C@H](O)[C@@H]1O)C1(OC2CCCCC2)CC1 ; O=C(OC[C@@]1(F)O[C@@H](n2ccc(=O)[nH]c2=O)[C@H](O)[C@@H]1O)C1(OC2CCCCC2)CCC1 ; O=C(OC[C@@]1(F)O[C@@H](n2ccc(=O)[nH]c2=O)[C@H](O)[C@@H]1O)C1(OC2CCCCC2)CCCC1 ; O=C(OC[C@@]1(F)O[C@@H](n2ccc(=O)[nH]c2=O)[C@H](O)[C@@H]1O)C1(OC2CCCCC2)CCCCC1]

or a pharmaceutically acceptable salt, thereof.

### Infectious Diseases

The compounds and pharmaceutical formulations provided herein can be used to treat viral infectious diseases.

Disclosed herein are methods of treating or preventing a viral infection comprising administering to a subject in need thereof an effective amount of a compound of Formulas XXIX-XXXIVb, or a pharmaceutical composition comprising a compound of Formulas XXIX-XXXIVb and a pharmaceutically acceptable excipient.

Also disclosed herein are methods of treating or preventing a viral infection comprising administering to a subject in need thereof an effective amount of a compound of Formulas XXIX-XXXIVb, or a pharmaceutical composition comprising a compound of Formulas XXIX-XXXIVb and a pharmaceutically acceptable excipient.

Disclosed are uses of a disclosed compound, e.g., one or more compound of Formulas XXIX-XXXIVb, or a pharmaceutically acceptable salt thereof, in the manufacture of a medicament for the treatment of a viral infectious disease.

Also disclosed are uses of a disclosed compound, e.g., one or more compound of Formulas XXIX-XXXIVb, or a pharmaceutically acceptable salt thereof, in the manufacture of a medicament for the treatment of a viral infectious disease.

Disclosed are methods of treating or preventing a viral infection comprising administering to a subject in need thereof an effective amount of a compound of Formulas XXIX-XXXIVb, or a pharmaceutical composition comprising a compound of Formulas XXIX-XXXIVb and a pharmaceutically acceptable excipient; wherein the subject is administered a loading dose of the pharmaceutical composition in a first treatment period; and wherein the subject is administered a treatment dose of the pharmaceutical composition in a second treatment period following the first treatment period.

Also disclosed are methods of treating or preventing a viral infection comprising administering to a subject in need thereof an effective amount of a compound of Formulas XXIX-XXXIVb, or a pharmaceutical composition comprising a compound of Formulas XXIX-XXXIVb and a pharmaceutically acceptable excipient; wherein the subject is administered a loading dose of the pharmaceutical composition in a first treatment period; and wherein the subject is administered a treatment dose of the pharmaceutical composition in a second treatment period following the first treatment period.

In exemplary embodiments, the first treatment period is days 1-5 following diagnosis of the viral infection or presentation for preventing the viral infection; the first treatment period is days 1-2 following diagnosis of the viral infection or presentation for preventing the viral infection; the first treatment period is day 1 following diagnosis of the viral infection or presentation for preventing the viral infection; and other periods as encompassed by the foregoing.

In exemplary embodiments, the first treatment period is 1 hour, 2 hours, 4 hours, 8 hours, 12 hours, 18 hours, or 24 hours prior to infection or exposure to a virus, and the second treatment period is a period of treatment following infection comprising 1-7 days of treatment following infection, e.g., for 1 day, 2 days, 3 days, 4 days, 5 days, 6 days, or 7 days after initial viral infection. As disclosed elsewhere herein, the dosing following infection may be daily, every other day, and the like.

In exemplary embodiments, the loading dose is about 1.1-fold to about 10-fold the treatment dose; the loading dose is about 1.5-fold to about 5-fold the treatment dose; the loading dose is about 1.5-fold to about 2.5-fold the treatment dose; and other loading doses as encompassed by the foregoing.

In exemplary embodiments, the loading dose is administered once daily, two times daily, three times daily, or four times daily, or alternatively, every other day, every third day and the like, and other periods of administration as contemplated within the foregoing. In some embodiments, the loading dose is administered at least twice daily. In further embodiments, the loading dose divided equally among the number of times administered daily. The during dosing of a loading dose, as described in the foregoing regarding doses per day, can be repeated and occur for 1, 2, 3, 4, 5, 6, 7, 8, 9, 10 or more days.

In exemplary embodiments, the treatment is delayed or late following initial diagnosis or presentation of the viral infection, that is, treatment initiates after some number of days after diagnosis or presentation of the viral infection, e.g., at days 1, 2, 3, 4, 5, 6, 7, 8, 9, 10 or more, and then continues for a suitable period of time, e.g., for 1, 2, 3, 4, 5, 6, 7, 8, 9, 10 or more days after initiation.

Examples of viral infections include but are not limited to, infections caused by RNA viruses (including negative stranded RNA viruses, positive stranded RNA viruses, double stranded RNA viruses and retroviruses) or DNA viruses. All strains, types, and subtypes of RNA viruses and DNA viruses are contemplated herein.

Examples of RNA viruses include, but are not limited to picornaviruses, which include aphthoviruses (for example, foot and mouth disease virus 0, A, C, Asia 1, SAT1, SAT2 and SAT3), cardioviruses (for example, encephalomycarditis virus and Theiller's murine encephalomyelitis virus), enteroviruses (for example polioviruses 1, 2 and 3, human enteroviruses A-D, bovine enteroviruses 1 and 2, human coxsackieviruses A1-A22 and A24, human coxsackieviruses B1-B5, human echoviruses 1-7, 9, 11-12, 24, 27, 29-33, human enteroviruses 68-71, porcine enteroviruses 8-10 and simian enteroviruses 1-18), erboviruses (for example, equine rhinitis virus), hepatovirus (for example human hepatitis A virus and simian hepatitis A virus), kobuviruses (for example, bovine kobuvirus and Aichi virus), parechoviruses (for example, human parechovirus 1 and human parechovirus 2), rhinovirus (for example, rhinovirus A, rhinovirus B, rhinovirus C, HRV16, HRV16 (VR-11757), HRV14 (VR-284), or HRVia (VR-1559), human rhinovirus 1-100 and bovine rhinoviruses 1-3) and teschoviruses (for example, porcine teschovirus).

Additional examples of RNA viruses include caliciviruses, which include noroviruses (for example, Norwalk virus), sapoviruses (for example, Sapporo virus), lagoviruses (for example, rabbit hemorrhagic disease virus and European brown hare syndrome) and vesiviruses (for example vesicular exanthema of swine virus and feline calicivirus). Other RNA viruses include astroviruses, which include mamastorviruses and avastroviruses. Togaviruses are also RNA viruses. Togaviruses include alphaviruses (for example, Chikungunya virus, Sindbis virus, Semliki Forest virus, Western equine encephalitis virus, Eastern Getah virus, Everglades virus, Venezuelan equine encephalitis virus, Ross River virus, Barmah Forest virus and Aura virus) and rubella viruses.

Other examples of RNA viruses are the coronaviruses, which include, human respiratory coronaviruses such as SARS-CoV (including SARS-CoV-2 and variants thereof including, but not limited to the more virulent strains that recently appeared in Brasil, known as P.1; the United Kingdom, known as 20I/501Y.V1, VOC 202012/01, or B.1.1.7; and in South Africa; known as 20H/501Y.V2 or B.1.351; as well as further varients and lineages that derive therefrom), HCoV-229E, HCoV-NL63 and HCoV-OC43. Coronaviruses also include bat SARS-like CoV, Middle East Respiratory Syndrome coronavirus (MERS), turkey coronavirus, chicken coronavirus, feline coronavirus and canine coronavirus. Coronaviruses are enveloped positive-sense RNA viruses that cause a large percentage of respiratory illness in humans. The two previous coronaviruses to emerge and cause human illness were SARS and MERS. There were more than 8,000 human cases of SARS with 774 deaths. Since 2012, there have been more than 2,500 cases of MERS with 919 deaths. In 2019 a new coronavirus, SARS-CoV-2, was discovered in humans in Wuhan, China and presently there is an ongoing pandemic with a large loss of life. SARS-CoV-2 is a highly pathogenic human pathogen. SARS-CoV-2 causes disease referred to as COVID-19. COVID-19 can include severe respiratory disease in humans, endothelial disease including stroke and neurological disease that includes dizziness, impaired consciousness, acute cerebrovascular disease, epilepsy, hyposmia, hypopsia, and neuralgia (medRxiv, 2020, 1-26). SARS-CoV-2 entry into the CNS may be promoted through viral interaction with ACE2 receptors after dissemination of the virus in the systemic circulation or across the cribriform plate.

Additional RNA viruses include arteriviruses (for example, equine arterivirus, porcine reproductive and respiratory syndrome virus, lactate dehyrogenase elevating virus of mice and simian hemorraghic fever virus). Other RNA viruses include the rhabdoviruses, which include lyssaviruses (for example, rabies, Lagos bat virus, Mokola virus, Duvenhage virus and European bat lyssavirus), vesiculoviruses (for example, VSV-Indiana, VSV-New Jersey, VSV-Alagoas, Piry virus, Cocal virus, Maraba virus, Isfahan virus and Chandipura virus), and ephemeroviruses (for example, bovine ephemeral fever virus, Adelaide River virus and Berrimah virus). Additional examples of RNA viruses include the filoviruses. These include the Marburg and Ebola viruses (for example, EBOV-Z, EBOV-S, EBOV-IC and EBOV-R).

The paramyxoviruses are also RNA viruses. Examples of these viruses are the rubulaviruses (for example, mumps, parainfluenza virus 5, human parainfluenza virus type 2, Mapuera virus and porcine rubulavirus), avulaviruses (for example, Newcastle disease virus), respoviruses (for example, Sendai virus, human parainfluenza virus type 1 and type 3, bovine parainfluenza virus type 3), henipaviruses (for example, Hendra virus and Nipah virus), morbilloviruses (for example, measles, Cetacean morvilliirus, Canine distemper virus, Peste des-petits-ruminants virus, Phocine distemper virus and Rinderpest virus), pneumoviruses (for example, human respiratory syncytial virus (RSV) A2, B1 and S2, bovine respiratory syncytial virus and pneumonia virus of mice), metapneumoviruses (for example, human metapneumovirus and avian metapneumovirus). Additional paramyxoviruses include Fer-de-Lance virus, Tupaia paramyxovirus, Menangle virus, Tioman virus, Beilong virus, J virus, Mossman virus, Salem virus and Nariva virus.

Additional RNA viruses include the orthomyxoviruses. These viruses include influenza viruses and strains (e.g., influenza A, influenza A strain A/Victoria/3/75, influenza A strain A/Puerto Rico/8/34, influenza A H1N1 (including but not limited to A/WS/33, A/NWS/33 and A/California/04/2009 strains), influenza B, influenza B strain Lee, and influenza C viruses) H2N2, H3N2, H5N1, H7N7, H1N2, H9N2, H7N2, H7N3 and H10N7), as well as avian influenza (for example, strains H5N1, H5N1 Duck/MN/1525/81, H5N2, H7N1, H7N7 and H9N2) thogotoviruses and isaviruses. Orthobunyaviruses (for example, Akabane virus, California encephalitis, Cache Valley virus, Snowshoe hare virus,) nairoviruses (for example, Nairobi sheep virus, Crimean-Congo hemorrhagic fever virus Group and Hughes virus), phleboviruses (for example, Candiru, Punta Toro, Rift Valley Fever, Sandfly Fever, Naples, Toscana, Sicilian and Chagres), and hantaviruses (for example, Hantaan, Dobrava, Seoul, Puumala, Sin Nombre, Bayou, Black Creek Canal, Andes and Thottapalayam) are also RNA viruses. Arenaviruses such as lymphocytic choriomeningitis virus, Lujo virus, Lassa fever virus, Argentine hemorrhagic fever virus, Bolivian hemorrhagic fever virus, Venezuelan hemorrhagic fever virus, SABV and WWAV are also RNA viruses. Boma disease virus is also an RNA virus. Hepatitis D (Delta) virus and hepatitis E are also RNA viruses.

Additional RNA viruses include reoviruses, rotaviruses, birnaviruses, chrysoviruses, cystoviruses, hypoviruses partitiviruses and totoviruses. Orbiviruses such as African horse sickness virus, Blue tongue virus, Changuinola virus, Chenuda virus, Chobar GorgeCorriparta virus, epizootic hemorraghic disease virus, equine encephalosis virus, Eubenangee virus, Ieri virus, Great Island virus, Lebombo virus, Orungo virus, Palyam virus, Peruvian Horse Sickness virus, St. Croix River virus, Umatilla virus, Wad Medani virus, Wallal virus, Warrego virus and Wongorr virus are also RNA viruses. Retroviruses include alpha retroviruses (for example, Rous sarcoma virus and avian leukemia virus), beta retroviruses (for example, mouse mammary tumor virus, Mason-Pfizer monkey virus and Jaagsiekte sheep retrovirus), gamma retroviruses (for example, murine leukemia virus and feline leukemia virus, deltra retroviruses (for example, human T cell leukemia viruses (HTLV-1, HTLV-2), bovine leukemia virus, STLV-1 and STLV-2), epsilon retriviruses (for example, Walleye dermal sarcoma virus and Walleye epidermal hyperplasia virus 1), reticuloendotheliosis virus (for example, chicken syncytial virus, lentiviruses (for example, human immunodeficiency virus (HIV) type 1, human immunodeficiency virus (HIV) type 2, human immunodeficiency virus (HIV) type 3, simian immunodeficiency virus, equine infectious anemia virus, feline immunodeficiency virus, caprine arthritis encephalitis virus and Visna maedi virus) and spumaviruses (for example, human foamy virus and feline syncytia-forming virus).

The virally encoded RNA-dependent-RNA polymerase (RdRp) forms a replication complex with other virally encoded proteins as well as host cell proteins and catalyzes RNA-template directed RNA synthesis. This protein is responsible for synthesizing antigenomic complementary RNA, genomic RNA for progeny viruses, and capped, nonpolyadenylated viral mRNA. Ribonucleoside analogs selectively inhibit the primary pathway of genetic information flow for these viruses (the copying of RNA from RNA) by acting on or through the virally encoded RdRp via their active 5′-triphosphate metabolite. A ribonucleoside analog (after phosphorylation to the corresponding 5′-triphosphate by host intracellular kinases) can act as a competitive, alternative substrate inhibitor of the RdRp and stop nascent chain RNA synthesis after incorporation; or, it can be utilized as a substrate by the RdRp and be incorporated into nascent chain RNA, rendering it non-functional by perturbing its secondary structure.

Examples of DNA viruses include polyomaviruses (for example, simian virus 40, simian agent 12, BK virus, JC virus, Merkel Cell polyoma virus, bovine polyoma virus and lymphotrophic papovavirus), papillomaviruses (for example, human papillomavirus, bovine papillomavirus, adenoviruses (for example, adenoviruses A-F, canine adenovirus type I, canined adeovirus type 2), circoviruses (for example, porcine circovirus and beak and feather disease virus (BFDV)), parvoviruses (for example, canine parvovirus), erythroviruses (for example, adeno-associated virus types 1-8), betaparvoviruses, amdoviruses, densoviruses, iteraviruses, brevidensoviruses, pefudensoviruses, herpes viruses 1, 2, 3, 4, 5, 6, 7 and 8 (for example, herpes simplex virus 1, herpes simplex virus 2, varicella-zoster virus, Epstein-Barr virus, cytomegalovirus, Kaposi's sarcoma associated herpes virus, human herpes virus-6 variant A, human herpes virus-6 variant B and cercophithecine herpes virus 1 (B virus)), poxviruses (for example, smallpox (variola), cowpox, monkeypox, vaccinia, Uasin Gishu, camelpox, psuedocowpox, pigeonpox, horsepox, fowlpox, turkeypox and swinepox), and hepadnaviruses (for example, hepatitis B and hepatitis B-like viruses). Chimeric viruses comprising portions of more than one viral genome are also contemplated herein.

In certain embodiments, the RNA viruses that can be treated by compounds and compositions of this disclosure include enteroviruses. The genus Enterovirus (EV) belonging to the Picornaviridae family comprises 13 species, of which seven are human viruses. Four of the species are: (1) EV-A such as coxsackievirus (CV)-A6, CV-A10, CV-A16 and EV-A71, (2) EV-B such as the CV-B viruses, echoviruses (ECHO) and CV-A9, (3) EV-C such as polioviruses (PV) and CV-A21, and (4) EV-D such as EV-D68 and EV-D70. Other species include rhinoviruses RV-A, RV—B and RV—C which are comprised of over 100 different numbered RVs. EV RNA contains a single open reading frame (ORF) flanked by two untranslated regions (UTRs), 5′ UTR and 3′ UTR. The ORF encodes a single polyprotein that is cleaved into P1, P2 and P3 proteins. The P1 protein is proteolytically cleaved to produce capsid proteins VP1-4. P2 and P3 are cleaved to produce non-structural (NS) proteins 2A, 2B, 2C and 3A, 3B, 3C, 3D, respectively. The role of the capsid proteins is to enclose the genetic material and to recognize cellular receptors during viral entry. The NS proteins are crucial for replication, translation and subversion of host cell machinery. The capsid proteins are suitable targets for antiviral development due to their role in cellular entry and uncoating of the genetic material.

The diverse viruses in the genus EV are known to cause a range of diseases such as hand, foot and mouth disease (HFMD), encephalitis, aseptic meningitis, myocarditis and various respiratory diseases. While some EV infections are mild, the symptoms can be severe in the very young and immunodeficient individuals. In recent years, viruses such as EV-A71 and CV-A16 have emerged as serious public health threats, as they have caused major outbreaks of HFMD in China and South East Asia. Additionally, EV-D68 has caused a large outbreak of severe lower respiratory infections in North America in 2014. Therefore, broad-spectrum antiviral drugs that could inhibit multiple EVs across the genus will be instrumental to overcome the public health burden caused by these EVs.

The compounds and compositions of this disclosure can be used to treat or prevent diseases caused by enterovirus and to reduce enterovirual burden. In addition, the compounds and compositions of this disclosure can be combined with other drugs to treat enterovirus as provided herein. Anasir et al., J Biomed Sci (2021) 28, 10:5-12 provides a review of enteroviruses and antiviral agents for treating the same, the disclosure of which is incorporated herein by reference in its entirety.

In certain embodiments, the disclosure relates to methods of treating or preventing a viral infection comprising administering an effective amount of a compound of Formulas XXIX-XXXIVb, or a pharmaceutical composition comprising a compound of Formulas XXIX-XXXIVb and a pharmaceutically acceptable excipient, disclosed herein to a subject in need thereof. In certain exemplary embodiments, a method of treating or preventing a Zika virus infection is provided, the method comprising administering an effective amount of a compound of Formulas XXIX-XXXIVb, or a pharmaceutical composition comprising a compound of Formulas XXIX-XXXIVb and a pharmaceutically acceptable excipient, disclosed herein to a subject in need thereof.

In certain embodiments, the viral infection is, or is caused by, an alphavirus, flavivirus or coronaviruses orthomyxoviridae or paramyxoviridae, or RSV, influenza, Powassan virus or filoviridae or ebola.

In certain embodiments, the viral infection is, or is caused by, a virus selected from MERS coronavirus, Eastern equine encephalitis virus, Western equine encephalitis virus, Venezuelan equine encephalitis virus, Ross River virus, Barmah Forest virus, Powassan virus, Zika virus, and Chikungunya virus. In certain exemplary embodiments, the viral infection is, or is caused by, a Zika virus.

In certain embodiments, the compound is administered by inhalation through the lungs.

In some embodiments, the subject is at risk of, exhibiting symptoms of, or diagnosed with influenza A virus including subtype H1N1, H3N2, H7N9, or H5N1, influenza B virus, influenza C virus, rotavirus A, rotavirus B, rotavirus C, rotavirus D, rotavirus E, human coronavirus, SARS coronavirus (including SARS-CoV-2 and variants thereof including, but not limited to the more virulent strains that recently appeared in Brasil, known as P.1; the United Kingdom, known as 20I/501Y.V1, VOC 202012/01, or B.1.1.7; and in South Africa; known as 20H1/501Y.V2 or B.1.351; as well as further varients and lineages that derive therefrom), MERS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), Dengue virus, Zika virus, chikungunya, Eastern equine encephalitis virus (EEEV), Western equine encephalitis virus (WEEV), Venezuelan equine encephalitis virus (VEEV), Ross River virus, Barmah Forest virus, yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV), The Human T-lymphotropic virus Type I (HTLV-1), Friend spleen focus-forming virus (SFFV) or Xenotropic MuLV-Related Virus (XMRV). In some embodiments, the subject is at risk of, exhibiting symptoms of, or diagnosed with a Zika virus infection.

In certain embodiments, the subject is diagnosed with influenza A virus including subtypes H1N1, H3N2, H7N9, H5N1 (low path), and H5N1 (high path) influenza B virus, influenza C virus, rotavirus A, rotavirus B, rotavirus C, rotavirus D, rotavirus E, SARS coronavirus (including SARS-CoV-2 and variants thereof including, but not limited to the more virulent strains that recently appeared in Brasil, known as P.1; the United Kingdom, known as 20I/501Y.V1, VOC 202012/01, or B.1.1.7; and in South Africa; known as 20H/501Y.V2 or B.1.351; as well as further varients and lineages that derive therefrom), MERS-CoV, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, parainfluenza viruses 1 and 3, rinderpest virus, chikungunya, eastern equine encephalitis virus (EEEV), Venezuelan equine encephalitis virus (VEEV), western equine encephalitis virus (WEEV), California encephalitis virus, Japanese encephalitis virus, Rift Valley fever virus (RVFV), hantavirus, Dengue virus serotypes 1, 2, 3 and 4, Zika virus, West Nile virus, Tacaribe virus, Junin, rabies virus, ebola virus, marburg virus, adenovirus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV). In certain embodiments, the subject is diagnosed with a Zika virus infection.

In certain embodiments, the subject is diagnosed with gastroenteritis, acute respiratory disease, severe acute respiratory syndrome, post-viral fatigue syndrome, viral hemorrhagic fevers, acquired immunodeficiency syndrome or hepatitis.

In exemplary embodiments, the disclosure relates to treating or preventing an infection by viruses, bacteria, fungi, protozoa, and parasites, e.g., comprising administering to a subject in need thereof an effective amount of a compound of Formulas XXIX-XXXIVb, or a pharmaceutical composition comprising a compound of Formulas XXIX-XXXIVb and a pharmaceutically acceptable excipient.

In some embodiments, the disclosure relates to methods of treating a viral infection comprising administering a compound herein to a subject that is diagnosed with, suspected of, or exhibiting symptoms of a viral infection.

Viruses are infectious agents that can typically replicate inside the living cells of organisms. Virus particles (virions) usually consist of nucleic acids, a protein coat, and in some cases an envelope of lipids that surrounds the protein coat. The shapes of viruses range from simple helical and icosahedral forms to more complex structures. Virally coded protein subunits will self-assemble to form a capsid, generally requiring the presence of the virus genome. Complex viruses can code for proteins that assist in the construction of their capsid. Proteins associated with nucleic acid are known as nucleoproteins, and the association of viral capsid proteins with viral nucleic acid is called a nucleocapsid.

Viruses are transmitted by a variety of methods including direct or bodily fluid contact, e.g., blood, tears, semen, preseminal fluid, saliva, milk, vaginal secretions, lesions; droplet contact, fecal-oral contact, or as a result of an animal bite or birth. A virus has either DNA or RNA genes and is called a DNA virus or a RNA virus respectively. A viral genome is either single-stranded or double-stranded. Some viruses contain a genome that is partially double-stranded and partially single-stranded. For viruses with RNA or single-stranded DNA, the strands are said to be either positive-sense (called the plus-strand) or negative-sense (called the minus-strand), depending on whether it is complementary to the viral messenger RNA (mRNA). Positive-sense viral RNA is identical to viral mRNA and thus can be immediately translated by the host cell. Negative-sense viral RNA is complementary to mRNA and thus must be converted to positive-sense RNA by an RNA polymerase before translation. DNA nomenclature is similar to RNA nomenclature, in that the coding strand for the viral mRNA is complementary to it (negative), and the non-coding strand is a copy of it (positive).

Antigenic shift, or reassortment, can result in novel strains. Viruses undergo genetic change by several mechanisms. These include a process called genetic drift where individual bases in the DNA or RNA mutate to other bases. Antigenic shift occurs when there is a major change in the genome of the virus. This can be a result of recombination or reassortment. RNA viruses often exist as quasispecies or swarms of viruses of the same species but with slightly different genome nucleoside sequences.

The genetic material within viruses, and the method by which the material is replicated, vary between different types of viruses. The genome replication of most DNA viruses takes place in the nucleus of the cell. If the cell has the appropriate receptor on its surface, these viruses enter the cell by fusion with the cell membrane or by endocytosis. Most DNA viruses are entirely dependent on the host DNA and RNA synthesizing machinery, and RNA processing machinery. Replication usually takes place in the cytoplasm. RNA viruses typically use their own RNA replicase enzymes to create copies of their genomes.

The Baltimore classification of viruses is based on the mechanism of mRNA production. Viruses must generate mRNAs from their genomes to produce proteins and replicate themselves, but different mechanisms are used to achieve this. Viral genomes may be single-stranded (ss) or double-stranded (ds), RNA or DNA, and may or may not use reverse transcriptase (RT). Additionally, ssRNA viruses may be either sense (plus) or antisense (minus). This classification places viruses into seven groups: I, dsDNA viruses (e.g. adenoviruses, herpesviruses, poxviruses); II, ssDNA viruses (plus)sense DNA (e.g. parvoviruses); III, dsRNA viruses (e.g. reoviruses); IV, (plus)ssRNA viruses (plus)sense RNA (e.g. picornaviruses, togaviruses); V, (minus)ssRNA viruses (minus)sense RNA (e.g. orthomyxoviruses, Rhabdoviruses); VI, ssRNA-RT viruses (plus)sense RNA with DNA intermediate in life-cycle (e.g. retroviruses); and VII, dsDNA-RT viruses (e.g. hepadnaviruses).

Human immunodeficiency virus (HIV) is a lentivirus (a member of the retrovirus family) that causes acquired immunodeficiency syndrome (AIDS). Lentiviruses are transmitted as single-stranded, positive-sense, enveloped RNA viruses. Upon entry of the target cell, the viral RNA genome is converted to double-stranded DNA by a virally encoded reverse transcriptase. This viral DNA is then integrated into the cellular DNA by a virally encoded integrase, along with host cellular co-factors. There are two species of HIV. HIV-1 is sometimes termed LAV or HTLV-III.

HIV infects primarily vital cells in the human immune system such as helper T cells (CD4+ T cells), macrophages, and dendritic cells. HIV infection leads to low levels of CD4+ T cells. When CD4+ T cell numbers decline below a critical level, cell-mediated immunity is lost, and the body becomes progressively more susceptible to other viral or bacterial infections.

Subjects with HIV typically develop malignancies associated with the progressive failure of the immune system.

The viral envelope is composed of two layers of phospholipids taken from the membrane of a human cell when a newly formed virus particle buds from the cell. Embedded in the viral envelope are proteins from the host cell and a HIV protein known as Env. Env contains glycoproteins gp120, and gp41. The RNA genome consists of at structural landmarks (LTR, TAR, RRE, PE, SLIP, CRS, and INS) and nine genes (gag, pol, and env, tat, rev, nef, vif, vpr, vpu, and sometimes a tenth tev, which is a fusion of tat env and rev) encoding 19 proteins. Three of these genes, gag, pol, and env, contain information needed to make the structural proteins for new virus particles. HIV-1 diagnosis is typically done with antibodies in an ELISA, Western blot, or immunoaffinity assays or by nucleic acid testing (e.g., viral RNA or DNA amplification).

HIV is typically treated with a combination of antiviral agent, e.g., two nucleoside-analogue reverse transcription inhibitors and one non-nucleoside-analogue reverse transcription inhibitor or protease inhibitor. The three-drug combination is commonly known as a triple cocktail. In certain embodiments, the disclosure relates to treating a subject diagnosed with HIV by administering a pharmaceutical composition disclosed herein in combination with two nucleoside-analogue reverse transcription inhibitors and one non-nucleoside-analogue reverse transcription inhibitor or protease inhibitor.

In certain embodiments, the disclosure relates to treating a subject by administering a compound disclosed herein, emtricitabine, tenofovir, and efavirenz. In certain embodiments, the disclosure relates to treating a subject by administering a compound disclosed herein, emtricitabine, tenofovir and raltegravir. In certain embodiments, the disclosure relates to treating a subject by administering a compound disclosed herein, emtricitabine, tenofovir, ritonavir and darunavir. In certain embodiments, the disclosure relates to treating a subject by administering a compound disclosed herein, emtricitabine, tenofovir, ritonavir and atazanavir.

Banana lectin (BanLec or BanLec-1) is one of the predominant proteins in the pulp of ripe bananas and has binding specificity for mannose and mannose-containing oligosaccharides. BanLec binds to the HIV-1 envelope protein gp120. In certain embodiments, the disclosure relates to treating viral infections, such as HIV, by administering a compound disclosed herein in combination with a banana lectin.

Therapeutic agents in some cases may suppress the virus for a long period of time. Typical medications are a combination of interferon alpha and ribavirin. Subjects may receive injections of pegylated interferon alpha. Genotypes 1 and 4 are less responsive to interferon-based treatment than are the other genotypes (2, 3, 5 and 6). In certain embodiments, the disclosure relates to treating a subject with HCV by administering a compound disclosed herein to a subject exhibiting symptoms or diagnosed with HCV. In certain embodiments, the compound is administered in combination with interferon alpha and another antiviral agent such as ribavirin, and/or a protease inhibitor such as telaprevir or boceprevir. In certain embodiments, the subject is diagnosed with genotype 2, 3, 5, or 6. In other embodiments, the subject is diagnosed with genotype 1 or 4.

In certain embodiments, the subject is diagnosed to have a virus by nucleic acid detection or viral antigen detection. Cytomegalovirus (CMV) belongs to the Betaherpesvirinae subfamily of Herpesviridae. In humans it is commonly known as HCMV or Human Herpesvirus 5 (HHV-5). Herpesviruses typically share a characteristic ability to remain latent within the body over long periods. HCMV infection may be life threatening for patients who are immunocompromised. In certain embodiments, the disclosure relates to methods of treating a subject diagnosed with cytomegalovirus or preventing a cytomegalovirus infection by administration of a compound disclosed herein. In certain embodiments, the subject is immunocompromised. In typical embodiments, the subject is an organ transplant recipient, undergoing hemodialysis, diagnosed with cancer, receiving an immunosuppressive drug, and/or diagnosed with an HIV-infection. In certain embodiments, the subject may be diagnosed with cytomegalovirus hepatitis, the cause of fulminant liver failure, cytomegalovirus retinitis (inflammation of the retina, may be detected by ophthalmoscopy), cytomegalovirus colitis (inflammation of the large bowel), cytomegalovirus pneumonitis, cytomegalovirus esophagitis, cytomegalovirus mononucleosis, polyradiculopathy, transverse myelitis, and subacute encephalitis. In certain embodiments, a compound disclosed herein is administered in combination with an antiviral agent such as valganciclovir or ganciclovir. In certain embodiments, the subject undergoes regular serological monitoring.

HCMV infections of a pregnant subject may lead to congenital abnormalities. Congenital HCMV infection ° Ccurs when the mother suffers a primary infection (or reactivation) during pregnancy. In certain embodiments, the disclosure relates to methods of treating a pregnant subject diagnosed with cytomegalovirus or preventing a cytomegalovirus infection in a subject at risk for, attempting to become, or currently pregnant by administering compound disclosed herein.

Subjects who have been infected with CMV typically develop antibodies to the virus. A number of laboratory tests that detect these antibodies to CMV have been developed. The virus may be cultured from specimens obtained from urine, throat swabs, bronchial lavages and tissue samples to detect active infection. One may monitor the viral load of CMV-infected subjects using PCR. CMV pp65 antigenemia test is an immunoaffinity based assay for identifying the pp65 protein of cytomegalovirus in peripheral blood leukocytes. CMV should be suspected if a patient has symptoms of infectious mononucleosis but has negative test results for mononucleosis and Epstein-Barr virus, or if they show signs of hepatitis, but have negative test results for hepatitis A, B, and C. A virus culture can be performed at any time the subject is symptomatic. Laboratory testing for antibody to CMV can be performed to determine if a subject has already had a CMV infection.

The enzyme-linked immunosorbent assay (or ELISA) is the most commonly available serologic test for measuring antibody to CMV. The result can be used to determine if acute infection, prior infection, or passively acquired maternal antibody in an infant is present. Other tests include various fluorescence assays, indirect hemagglutination, (PCR), and latex agglutination. An ELISA technique for CMV-specific IgM is available.

Hepatitis B virus is a hepadnavirus. The virus particle, (virion) consists of an outer lipid envelope and an icosahedral nucleocapsid core composed of protein. The genome of HBV is made of circular DNA, but the DNA is not fully double-stranded. One end of the strand is linked to the viral DNA polymerase. The virus replicates through an RNA intermediate form by reverse transcription. Replication typically takes place in the liver where it causes inflammation (hepatitis). The virus spreads to the blood where virus-specific proteins and their corresponding antibodies are found in infected people. Blood tests for these proteins and antibodies are used to diagnose the infection.

Hepatitis B virus gains entry into the cell by endocytosis. Because the virus multiplies via RNA made by a host enzyme, the viral genomic DNA has to be transferred to the cell nucleus by host chaperones. The partially double stranded viral DNA is then made fully double stranded and transformed into covalently closed circular DNA (cccDNA) that serves as a template for transcription of viral mRNAs. The virus is divided into four major serotypes (adr, adw, ayr, ayw) based on antigenic epitopes presented on its envelope proteins, and into eight genotypes (A-H) according to overall nucleotide sequence variation of the genome.

The hepatitis B surface antigen (HBsAg) is typically used to screen for the presence of this infection. It is the first detectable viral antigen to appear during infection. However, early in an infection, this antigen may not be present and it may be undetectable later in the infection if it is being cleared by the host. The infectious virion contains an inner “core particle” enclosing viral genome. The icosahedral core particle is made of core protein, alternatively known as hepatitis B core antigen, or HBcAg. IgM antibodies to the hepatitis B core antigen (anti-HBc IgM) may be used as a serological marker. Hepatitis B e antigen (HBeAg) may appear. The presence of HBeAg in the serum of the host is associated with high rates of viral replication. Certain variants of the hepatitis B virus do not produce the ‘e’ antigen,

If the host is able to clear the infection, typically the HBsAg will become undetectable and will be followed by IgG antibodies to the hepatitis B surface antigen and core antigen, (anti-HBs and anti HBc IgG). The time between the removal of the HBsAg and the appearance of anti-HBs is called the window period. A person negative for HBsAg but positive for anti-HBs has either cleared an infection or has been vaccinated previously. Individuals who remain HBsAg positive for at least six months are considered to be hepatitis B carriers. Carriers of the virus may have chronic hepatitis B, which would be reflected by elevated serum alanine aminotransferase levels and inflammation of the liver that may be identified by biopsy. Nucleic acid (PCR) tests have been developed to detect and measure the amount of HBV DNA in clinical specimens.

Acute infection with hepatitis B virus is associated with acute viral hepatitis. Acute viral hepatitis typically begins with symptoms of general ill health, loss of appetite, nausea, vomiting, body aches, mild fever, dark urine, and then progresses to development of jaundice. Chronic infection with hepatitis B virus may be either asymptomatic or may be associated with a chronic inflammation of the liver (chronic hepatitis), possibly leading to cirrhosis. Having chronic hepatitis B infection increases the incidence of hepatocellular carcinoma (liver cancer).

During HBV infection, the host immune response causes both hepatocellular damage and viral clearance. The adaptive immune response, particularly virus-specific cytotoxic T lymphocytes (CTLs), contributes to most of the liver injury associated with HBV infection. By killing infected cells and by producing antiviral cytokines capable of purging HBV from viable hepatocytes, CTLs eliminate the virus. Although liver damage is initiated and mediated by the CTLs, antigen-nonspecific inflammatory cells can worsen CTL-induced immunopathology, and platelets activated at the site of infection may facilitate the accumulation of CTLs in the liver.

Therapeutic agents can stop the virus from replicating, thus minimizing liver damage. In certain embodiments, the disclosure relates to methods of treating a subject diagnosed with HBV by administering a compound disclosed herein. In certain embodiments, the subject is immunocompromised. In certain embodiments, the compound is administered in combination with another antiviral agent such as lamivudine, adefovir, tenofovir, telbivudine, and entecavir, and/or immune system modulators interferon alpha-2a and pegylated interferon alpha-2a (Pegasys). In certain embodiments, the disclosure relates to preventing an HBV infection in an immunocompromised subject at risk of infection by administering a pharmaceutical composition disclosed herein and optionally one or more antiviral agents. In certain embodiments, the subject is at risk of an infection because the sexual partner of the subject is diagnosed with HBV.

In certain embodiments, pharmaceutical compositions disclosed herein are administered in combination with a second antiviral agent, such as ABT-450, ABT-267, ABT-333, ABT-493, ABT-530, abacavir, acyclovir, acyclovir, adefovir, amantadine, amprenavir, ampligen, arbidol, atazanavir, atripla, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, or zidovudine and combinations thereof.

In certain embodiments, pharmaceutical compositions disclosed herein can be coformulated and administered in combination with a second antiviral agent selected from:

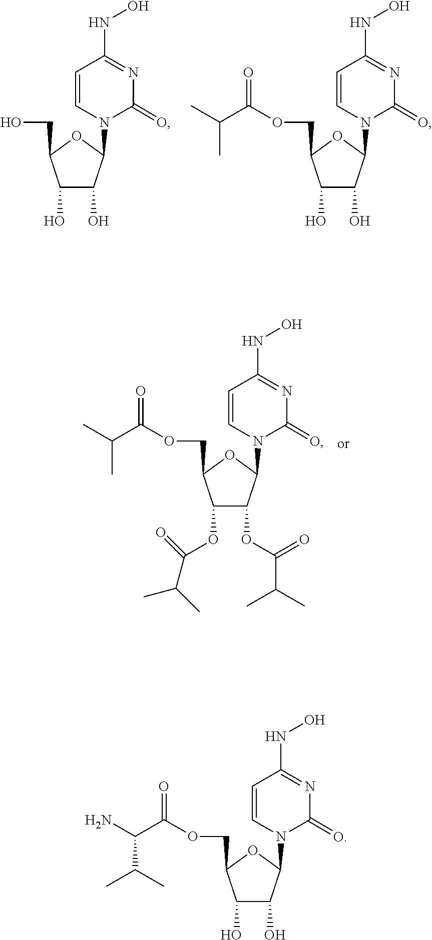

In certain embodiments,

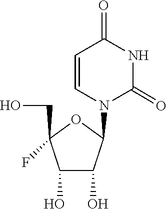 [SMILES: O=c1ccn([C@@H]2O[C@](F)(CO)[C@@H](O)[C@H]2O)c(=O)[nH]1]

can be coformulated and administered in combination with a second antiviral agent selected from:

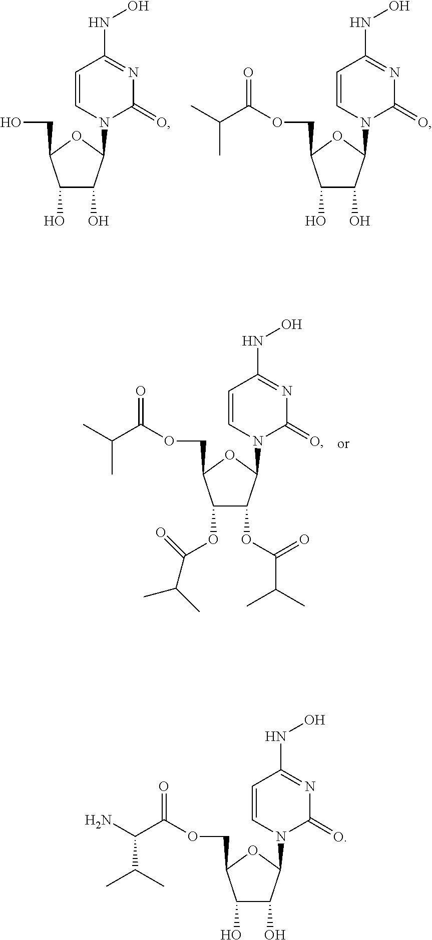

In certain embodiments,

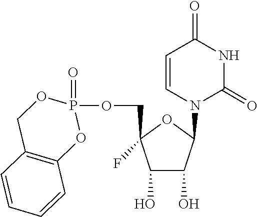 [SMILES: O=c1ccn([C@@H]2O[C@](F)(COP3(=O)OCc4ccccc4O3)[C@@H](O)[C@H]2O)c(=O)[nH]1]

can be coformulated and administered in combination with a second antiviral agent selected from:

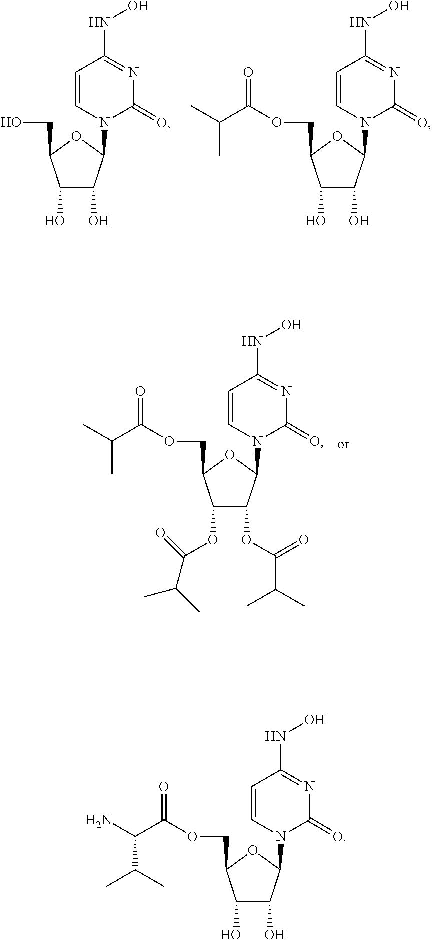

In certain embodiments, pharmaceutical compositions disclosed herein can be coformulated and administered in combination with a second antiviral agent selected from WO 2016/106050 or WO 2017/156380.

In certain embodiments,

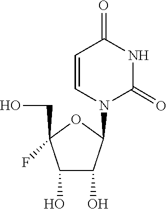 [SMILES: O=c1ccn([C@@H]2O[C@](F)(CO)[C@@H](O)[C@H]2O)c(=O)[nH]1]

can be coformulated and administered in combination with a second antiviral agent selected from WO 2016/106050 or WO 2017/156380.

In certain embodiments,

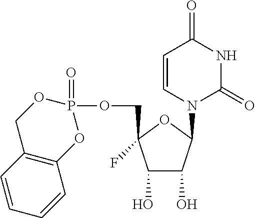 [SMILES: O=c1ccn([C@@H]2O[C@](F)(COP3(=O)OCc4ccccc4O3)[C@@H](O)[C@H]2O)c(=O)[nH]1]

can be coformulated and administered in combination with a second antiviral agent selected from WO 2016/106050 or WO 2017/156380.

In exemplified embodiments,

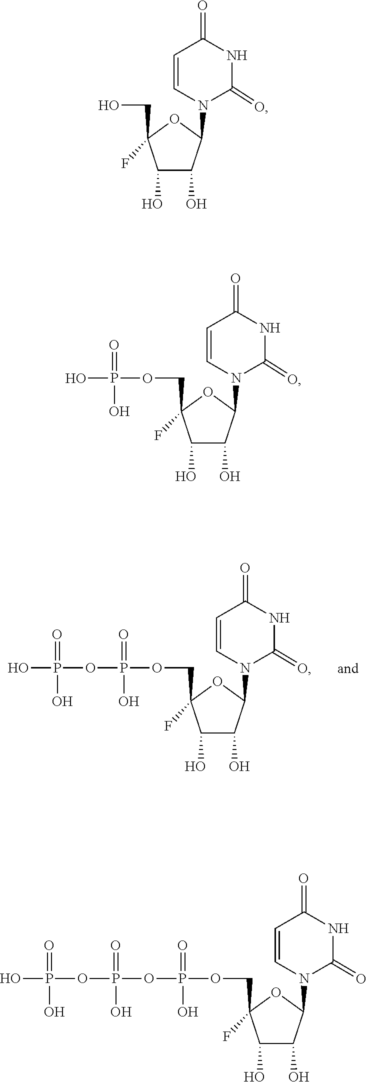 [SMILES: O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; "and" ; O=c1ccn([C@@H]2O[C@](F)(COP(=O)(O)OP(=O)(O)OP(=O)(O)O)[C@@H](O)[C@H]2O)c(=O)[nH]1]

can be combined with

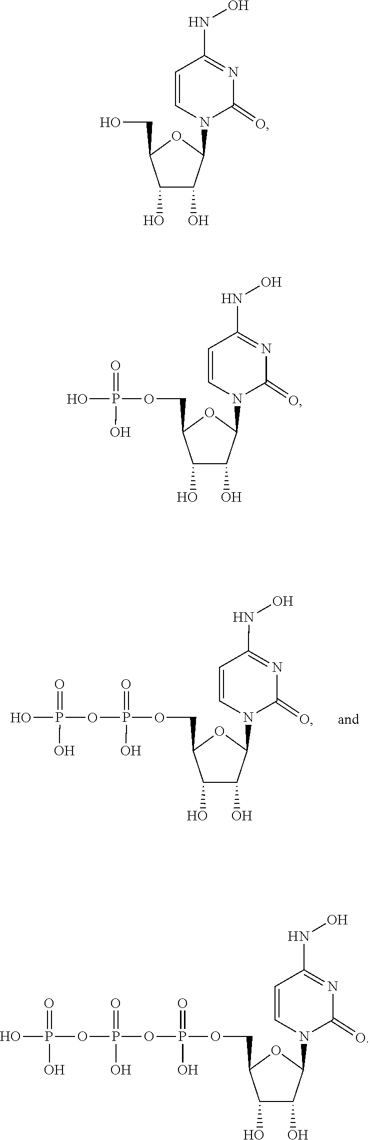 [SMILES: O=c1nc(NO)ccn1[C@@H]1O[C@H](CO)[C@@H](O)[C@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; "and" ; O=c1nc(NO)ccn1[C@@H]1O[C@H](COP(=O)(O)OP(=O)(O)OP(=O)(O)O)[C@@H](O)[C@H]1O]

In exemplified embodiments,

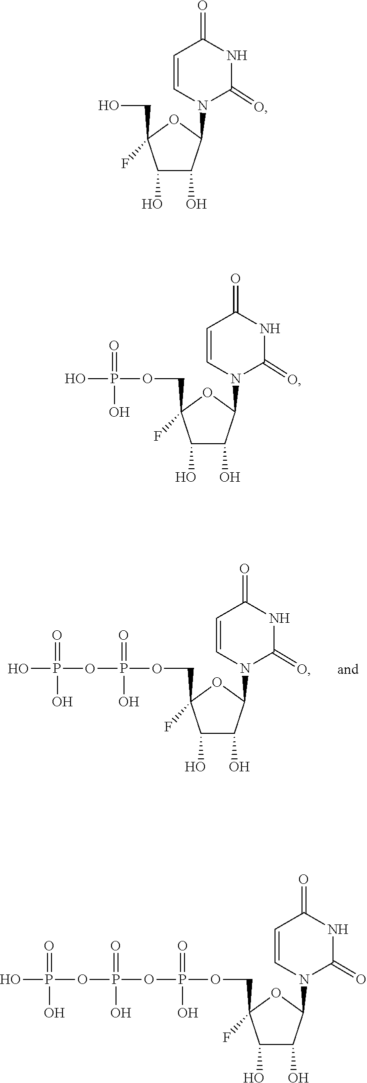 [SMILES: O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; "and" ; O=c1ccn([C@@H]2O[C@](F)(COP(=O)(O)OP(=O)(O)OP(=O)(O)O)[C@@H](O)[C@H]2O)c(=O)[nH]1]

can be combined with

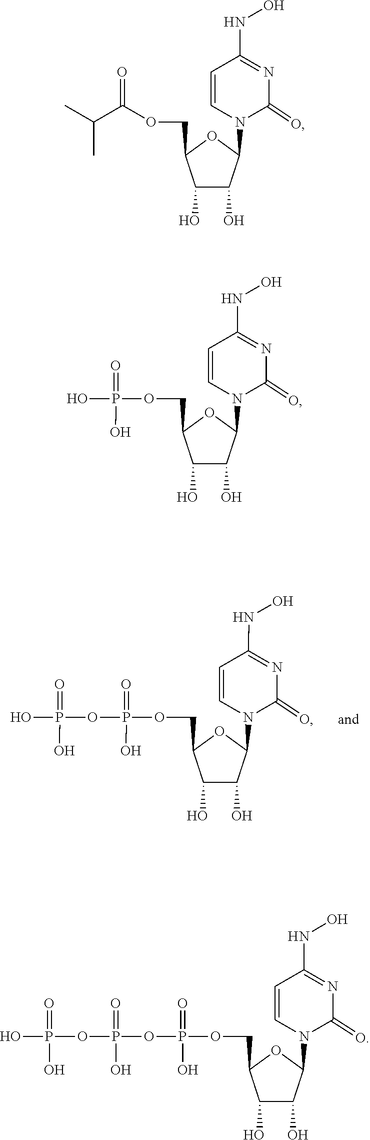 [SMILES: CC(C)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; "and" ; O=c1nc(NO)ccn1[C@@H]1O[C@H](COP(=O)(O)OP(=O)(O)OP(=O)(O)O)[C@@H](O)[C@H]1O]

In exemplified embodiments,

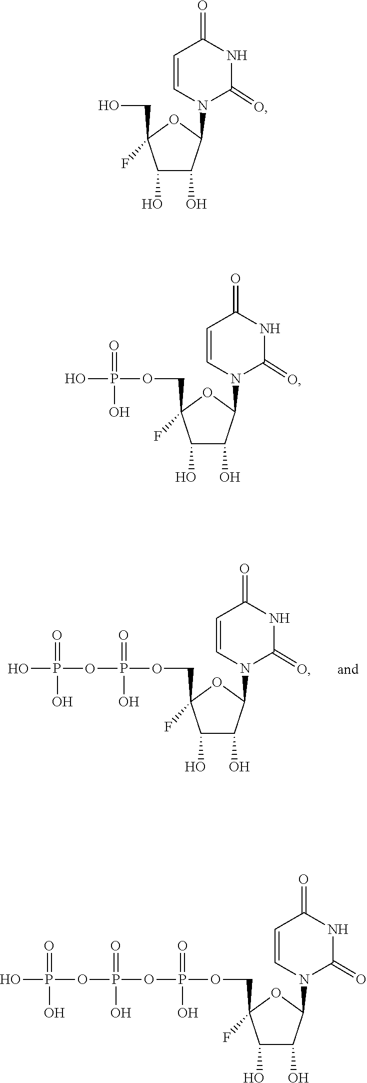 [SMILES: O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; "and" ; O=c1ccn([C@@H]2O[C@](F)(COP(=O)(O)OP(=O)(O)OP(=O)(O)O)[C@@H](O)[C@H]2O)c(=O)[nH]1]

can be combined with

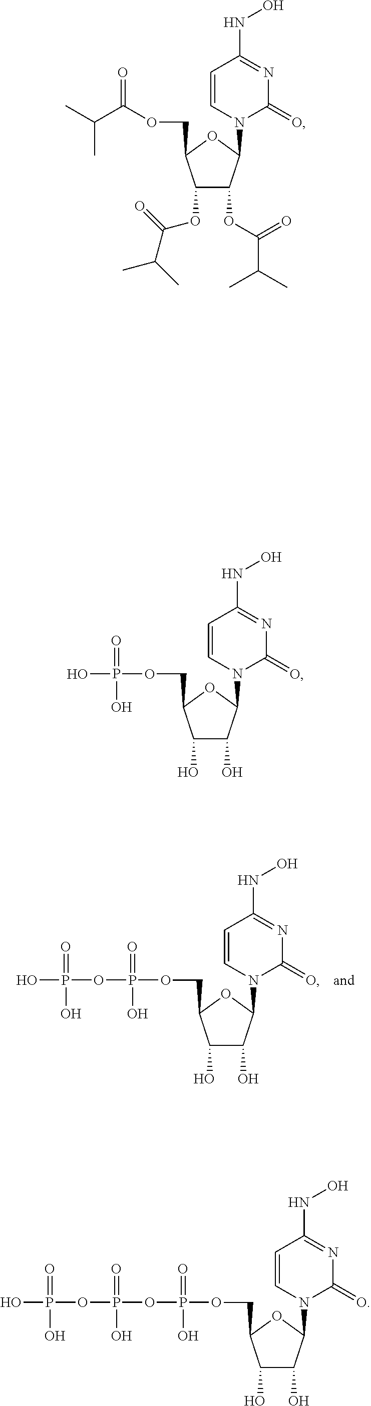 [SMILES: CC(C)C(=O)OC[C@H]1O[C@@H](n2ccc(NO)nc2=O)[C@H](OC(=O)C(C)C)[C@@H]1OC(=O)C(C)C ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

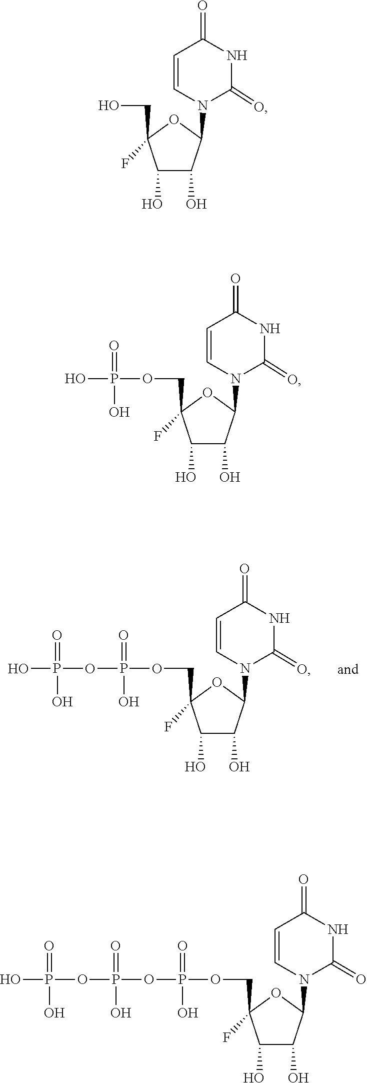 [SMILES: O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; "and" ; O=c1ccn([C@@H]2O[C@](F)(COP(=O)(O)OP(=O)(O)OP(=O)(O)O)[C@@H](O)[C@H]2O)c(=O)[nH]1]

can be combined with

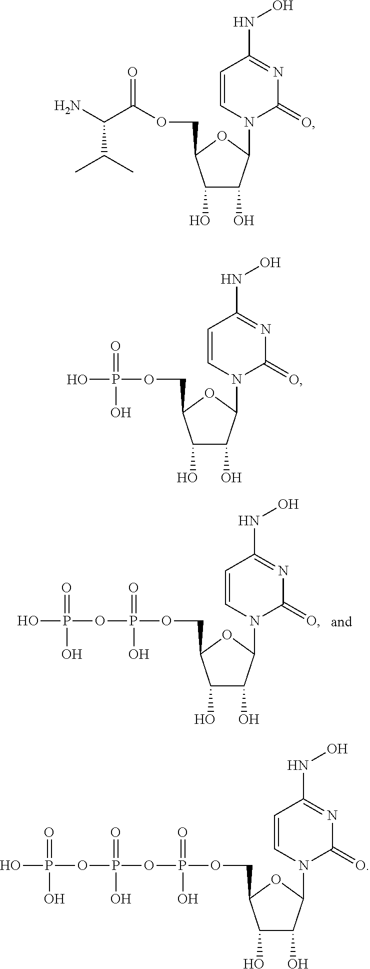 [SMILES: CC(C)[C@H](N)C(=O)OC[C@H]1OC(n2ccc(NO)nc2=O)[C@H](O)[C@@H]1O ; O=c1nc(NO)ccn1C1O[C@H](COP(=O)(O)O)[C@@H](O)[C@H]1O ; O=c1nc(NO)ccn1C1O[C@H](COP(=O)(O)OP(=O)(O)O)[C@@H](O)[C@H]1O ; O=c1nc(NO)ccn1C1O[C@H](COP(=O)(O)OP(=O)(O)OP(=O)(O)O)[C@@H](O)[C@H]1O]

In exemplified embodiments,

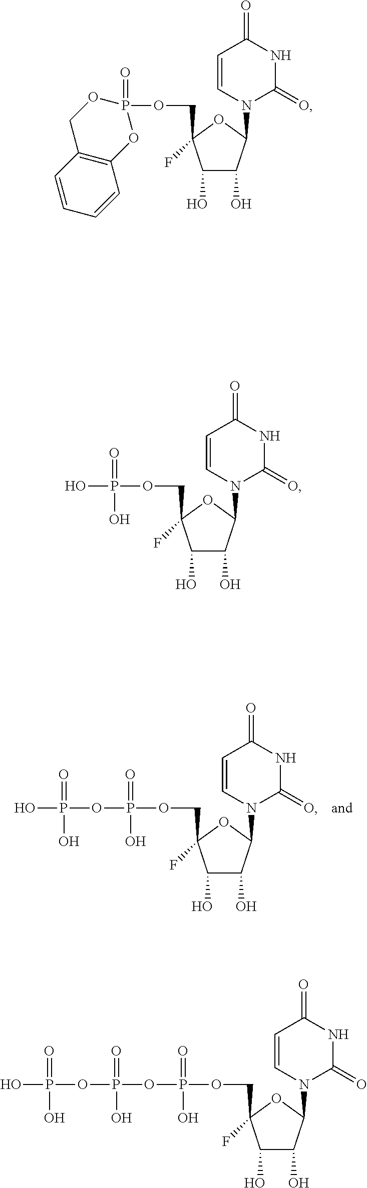 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

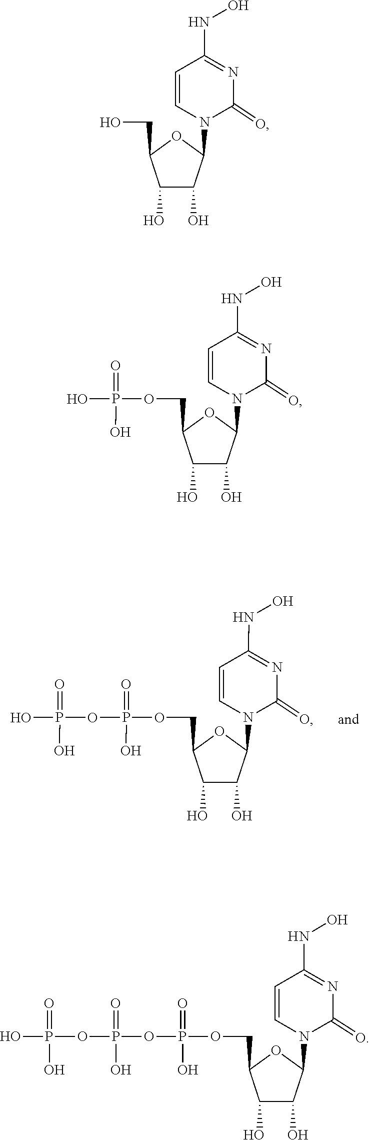 [SMILES: O=c1nc(NO)ccn1[C@@H]1O[C@H](CO)[C@@H](O)[C@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; "and" ; O=c1nc(NO)ccn1[C@@H]1O[C@H](COP(=O)(O)OP(=O)(O)OP(=O)(O)O)[C@@H](O)[C@H]1O]

In exemplified embodiments,

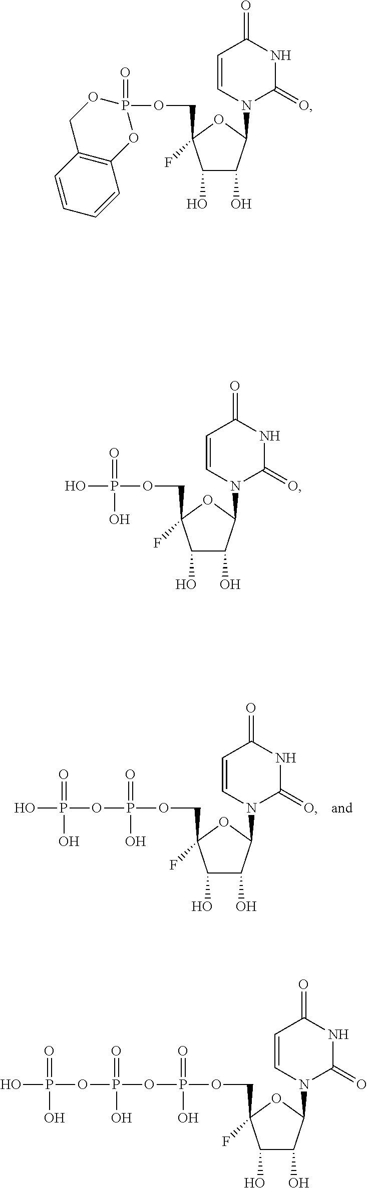 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

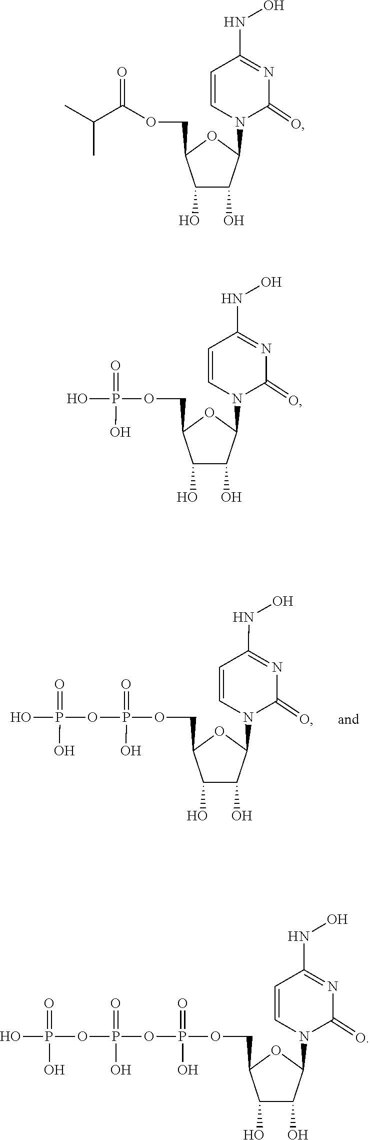 [SMILES: CC(C)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; "and" ; O=c1nc(NO)ccn1[C@@H]1O[C@H](COP(=O)(O)OP(=O)(O)OP(=O)(O)O)[C@@H](O)[C@H]1O]

In exemplified embodiments,

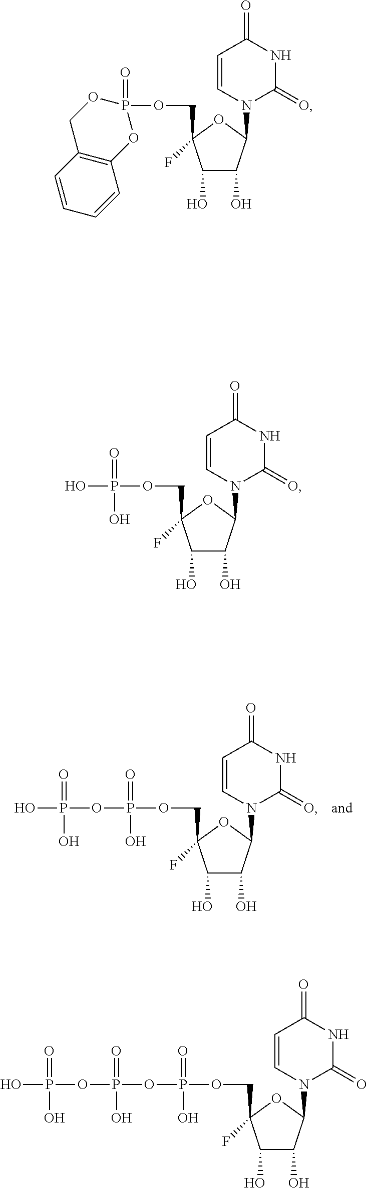 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

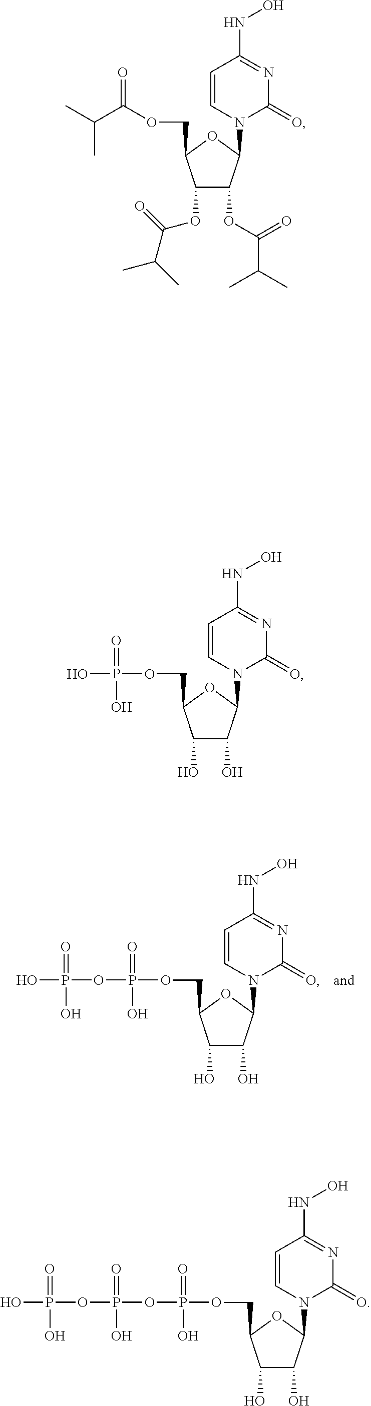 [SMILES: CC(C)C(=O)OC[C@H]1O[C@@H](n2ccc(NO)nc2=O)[C@H](OC(=O)C(C)C)[C@@H]1OC(=O)C(C)C ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; O=c1nc(NO)ccn1[C@@H]1O[C@H](COP(=O)(O)OP(=O)(O)OP(=O)(O)O)[C@@H](O)[C@H]1O]

In exemplified embodiments,

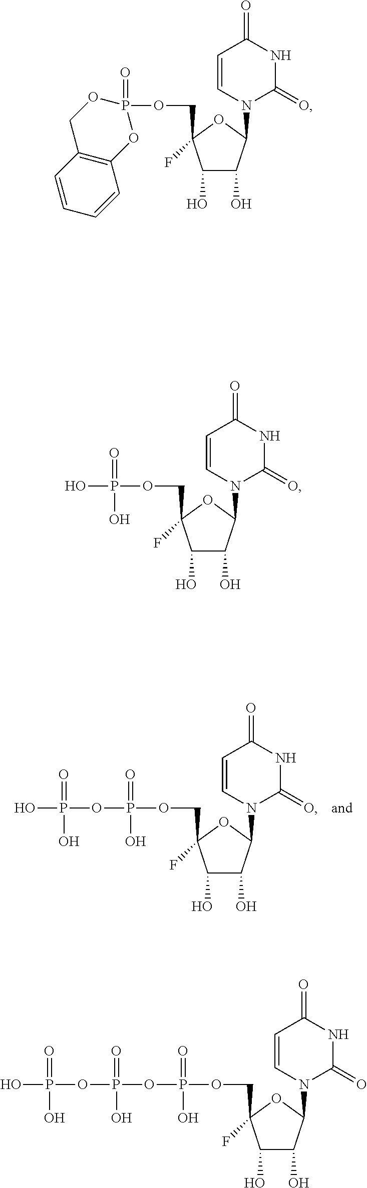 [SMILES: O=c1ccn([C@@H]2O[C@](F)(COP3(=O)OCc4ccccc4O3)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

can be combined with

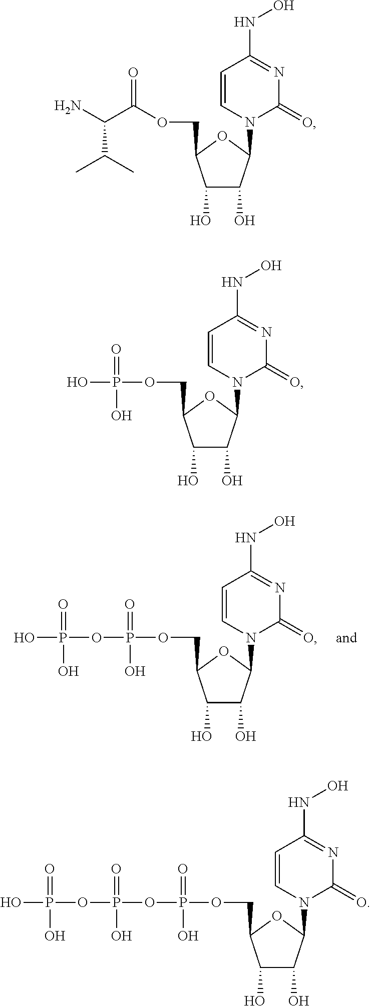 [SMILES: CC(C)[C@H](N)C(=O)OC[C@H]1O[C@@H](n2ccc(NO)nc2=O)[C@H](O)[C@@H]1O ; O=c1nc(NO)ccn1[C@@H]1O[C@H](COP(=O)(O)O)[C@@H](O)[C@H]1O ; O=c1nc(NO)ccn1[C@@H]1O[C@H](COP(=O)(O)OP(=O)(O)O)[C@@H](O)[C@H]1O ; "and" ; O=c1nc(NO)ccn1[C@@H]1O[C@H](COP(=O)(O)OP(=O)(O)OP(=O)(O)O)[C@@H](O)[C@H]1O]

In exemplified embodiments,

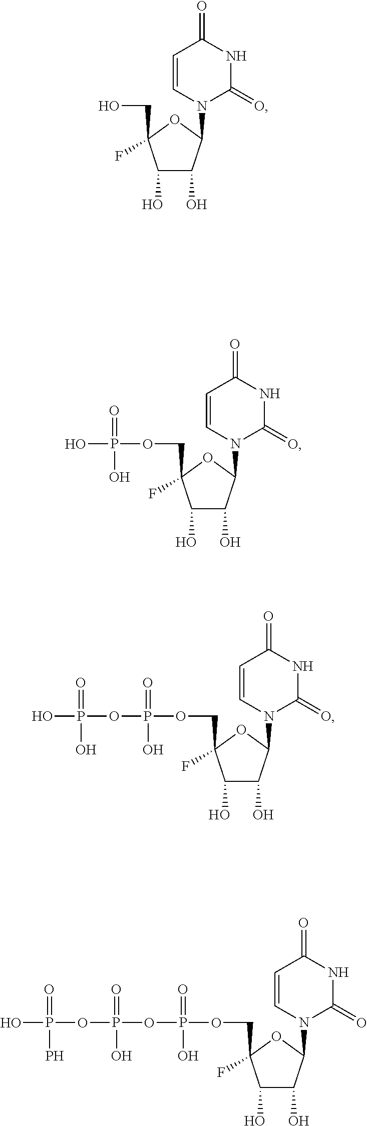

or a pharmaceutical or
physiological salt thereof with

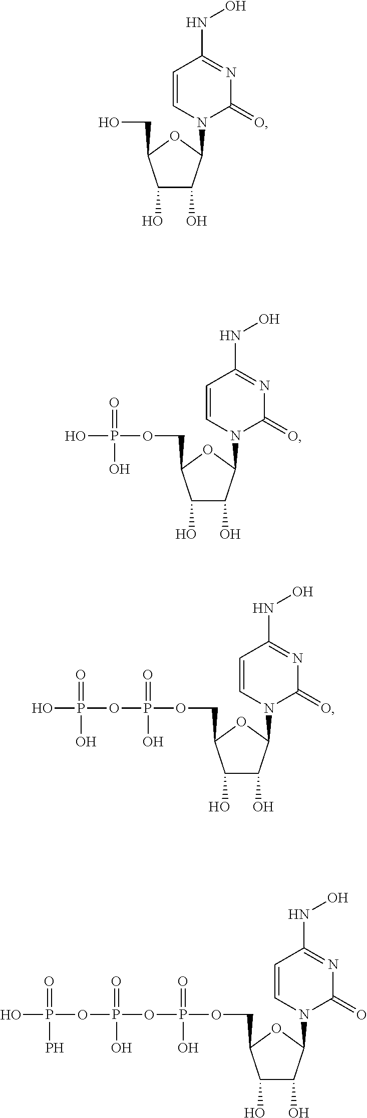

or a pharmaceutical or physiological salt thereof can be found in combination in host cells, tissues, and/or organs that are and are not infected with a virus.

In exemplified embodiments,

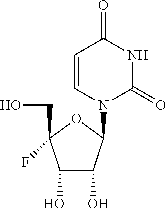 [SMILES: O=c1ccn([C@@H]2O[C@](F)(CO)[C@@H](O)[C@H]2O)c(=O)[nH]1]

or a pharmaceutical or physiological salt thereof can be found in combination with

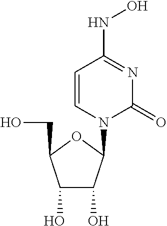 [SMILES: O=c1nc(NO)ccn1[C@@H]1O[C@H](CO)[C@@H](O)[C@H]1O]

or a pharmaceutical or physiological salt thereof in host plasma or whole blood.

In exemplified embodiments,

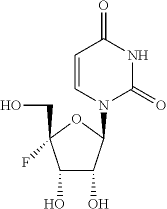 [SMILES: O=c1ccn([C@@H]2O[C@](F)(CO)[C@@H](O)[C@H]2O)c(=O)[nH]1]

or a pharmaceutical or physiological salt thereof can be found in combination with

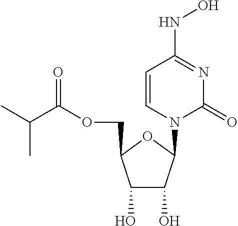 [SMILES: CC(C)C(=O)OC[C@H]1O[C@@H](n2ccc(NO)nc2=O)[C@H](O)[C@@H]1O]

or a pharmaceutical or physiological salt thereof in host plasma or whole blood.

In exemplified embodiments,

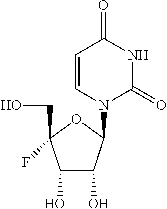 [SMILES: O=c1ccn([C@@H]2O[C@](F)(CO)[C@@H](O)[C@H]2O)c(=O)[nH]1]

or a pharmaceutical or physiological salt thereof can be found in combination with

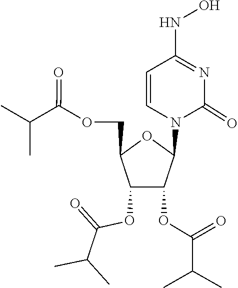 [SMILES: CC(C)C(=O)OC[C@H]1O[C@@H](n2ccc(NO)nc2=O)[C@H](OC(=O)C(C)C)[C@@H]1OC(=O)C(C)C]

or a pharmaceutical or physiological salt thereof in host plasma or whole blood.

In exemplified embodiments,

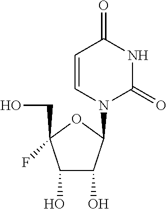 [SMILES: O=c1ccn([C@@H]2O[C@](F)(CO)[C@@H](O)[C@H]2O)c(=O)[nH]1]

or a pharmaceutical or physiological salt thereof can be found in combination with

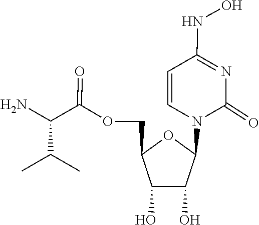 [SMILES: CC(C)[C@H](N)C(=O)OC[C@H]1O[C@@H](n2ccc(NO)nc2=O)[C@H](O)[C@@H]1O]

or a pharmaceutical or physiological salt thereof in host plasma or whole blood.

In exemplified embodiments,

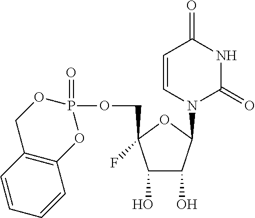 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

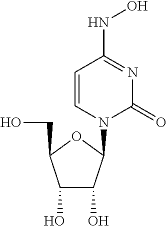 [SMILES: O=c1nc(NO)ccn1[C@@H]1O[C@H](CO)[C@@H](O)[C@H]1O]

or a pharmaceutical or physiological salt thereof in host plasma or whole blood.

In exemplified embodiments,

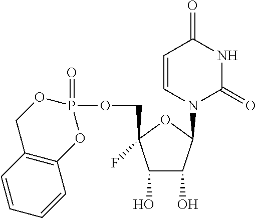 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

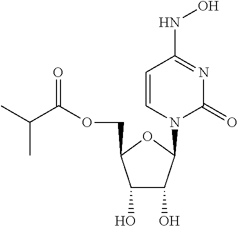 [SMILES: CC(C)C(=O)OC[C@H]1O[C@@H](n2ccc(NO)nc2=O)[C@H](O)[C@@H]1O]

or a pharmaceutical or physiological salt thereof in host plasma or whole blood.

In exemplified embodiments,

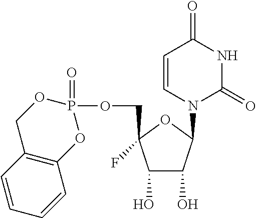 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof can be found in combination with

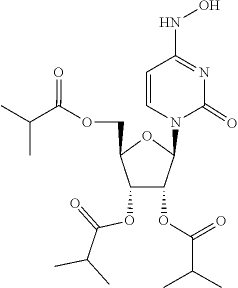 [SMILES: CC(C)C(=O)OC[C@H]1O[C@@H](n2ccc(NO)nc2=O)[C@H](OC(=O)C(C)C)[C@@H]1OC(=O)C(C)C]

or a pharmaceutical or physiological salt thereof in host plasma or whole blood.

In exemplified embodiments,

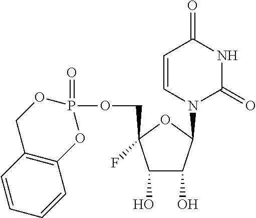 [SMILES: O=c1ccn([C@@H]2O[C@](F)(COP3(=O)OCc4ccccc4O3)[C@@H](O)[C@H]2O)c(=O)[nH]1]

or a pharmaceutical or
physiological salt thereof can be found in combination with

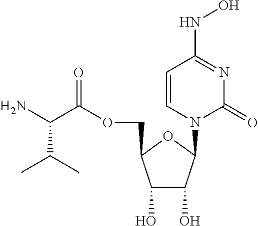 [SMILES: CC(C)[C@H](N)C(=O)OC[C@H]1O[C@@H](n2ccc(NO)nc2=O)[C@H](O)[C@@H]1O]

or a pharmaceutical or physiological salt thereof in host plasma or whole blood.

In yet another aspect, the at least two direct acting antiviral agents comprises a drug combination selected from the group consisting of: a compound of this invention, with one or more of ABT-450 and/or ABT-267, and/or ABT-333, and/or ABT-493, and/or ABT-530; a novel compound of this invention with a compound disclosed in any of US 2010/0144608; U.S. 61/339,964; US 2011/0312973; WO 2009/039127; US 2010/0317568; 2012/151158; US 2012/0172290; WO 2012/092411; WO 2012/087833; WO 2012/083170; WO 2009/039135; US 2012/0115918; WO 2012/051361; WO 2012/009699; WO 2011/156337; US 2011/0207699; WO 2010/075376; U.S. Pat. No. 7,910,595; WO 2010/120935; WO 2010/111437; WO 2010/111436; US 2010/0168384 or US 2004/0167123; a compound of this invention with one or more of Simeprevir, and/or GSK805; a compound of this invention with one or more of Asunaprevir, and/or Daclastavir, and/or BMS-325; a compound of this invention with one or more of GS-9451, and/or Ledisasvir and/or Sofosbuvir, and/or GS-9669; a compound of this invention with one or more of ACH-2684, and/or ACH-3102, and/or ACH-3422; a compound of this invention with one or more of Boceprevir, and/or MK-8742; a compound of this invention with one or more of Faldaprevir and/or Deleobuvir; a compound of this invention with PPI-668; a compound of this invention with one or more of telaprevir and/or VX-135; a compound of this invention with one or more of Samatasvir and/or IDX-437; a compound of this invention with PSI-7977 and/or PSI-938, a compound of this invention with BMS-790052 and/or BMS-650032; a compound of this invention with GS-5885 and/or GS-9451; a compound of this invention with GS-5885, GS-9190 and/or GS-9451; a compound of this invention in combination with BI-201335 and/or BI-27127; a compound of this invention in combination with telaprevir and/or VX-222; a compound of this invention combination with PSI-7977 and/or TMC-435; and a compound of this invention in combination with danoprevir and/or R7128.

In one aspect of the disclosure, an “infection” or “bacterial infection” refers to an infection caused by Acinetobacter spp, BacteroidesBacteroides spp, BurkholderiaBurkholderia spp, Campylobacter spp, Chlamydia spp, Chlamydophila spp, Clostridium spp, Enterobacter spp, Enterococcus spp, Escherichia spp, Fusobacterium spp, Gardnerella spp, Haemophilus spp, Helicobacter spp, Klebsiella spp, Legionella spp, Moraxella spp, Morganella spp, Mycoplasma spp, Neisseria spp, Peptococcus spp, PeptostreptococcusPeptostreptococcus spp, Proteus spp, Pseudomonas spp, SalmonellaSalmonella spp, Serratia spp., StaphylococcusStaphylococcus spp, Streptococcus spp, Stenotrophomonas spp, or Ureaplasma spp.

In one aspect of the disclosure, an “infection” or “bacterial infection” refers to an infection caused by Acinetobacter baumanii, Acinetobacter haemolyticus, Acinetobacter junii, Acinetobacter johnsonii, Acinetobacter lwoffi, Bacteroides bivius, Bacteroides fragilis, Burkholderia cepacia, Campylobacter jejuni, Chlamydia pneumoniae, Chlamydia urealyticus, Chlamydophila pneumoniae, Clostridium difficile, Enterobacter aerogenes, Enterobacter cloacae, Enterococcus faecalis, Enterococcus faecium, Escherichia coli, Gardnerella vaginalis, Haemophilus par influenzae, Haemophilus influenzae, Helicobacter pylori, Klebsiella pneumoniae, Legionella pneumophila, methicillin-resistant Staphylococcus aureus, methicillin-susceptible Staphylococcus aureus, Moraxella catarrhalis, Morganella morganii, Mycoplasma pneumoniae, Neisseria gonorrhoeae, penicillin-resistant Streptococcus pneumoniae, penicillin-susceptible Streptococcus pneumoniae, PeptostreptococcusPeptostreptococcus magnus, Peptostreptococcus micros, Peptostreptococcus anaerobius, Peptostreptococcus asaccharolyticus, Peptostreptococcus prevotii, Peptostreptococcus tetradius, Peptostreptococcus vaginalis, Proteus mirabilis, Pseudomonas aeruginosa, quinolone-resistant Staphylococcus aureus, quinolone-resistant Staphylococcus epidermis, SalmonellaSalmonella typhi, Salmonella paratyphi, Salmonella enteritidis, Salmonella typhimurium, Serratia marcescens, Staphylococcus aureus, Staphylococcus epidermidis, Staphylococcus saprophyticus, Streptococcus agalactiae, Streptococcus pneumoniae, Streptococcus pyogenes, Stenotrophomonas maltophilia, Ureaplasma urealyticum, vancomycin-resistant Enterococcus faecium, vancomycin-resistant Enterococcus faecalis, vancomycin-resistant Staphylococcus aureus, vancomycin-resistant Staphylococcus epidermis, Mycobacterium tuberculosis, Clostridium perfringens, Klebsiella oxytoca, Neisseria meningitidis, Proteus vulgaris, or coagulase-negative StaphylococcusStaphylococcus (including Staphylococcus lugdunensis, Staphylococcus capitis, Staphylococcus hominis, or Staphylococcus saprophytic).

In one aspect of the disclosure “infection” or “bacterial infection” refers to aerobes, obligate anaerobes, facultative anaerobes, gram-positive bacteria, gram-negative bacteria, gram-variable bacteria, or atypical respiratory pathogens.

In some embodiments, the disclosure relates to treating a bacterial infection such as a gynecological infection, a respiratory tract infection (RTI), a sexually transmitted disease, or a urinary tract infection.

In some embodiments, the disclosure relates to treating a bacterial infection such as an infection caused by drug resistant bacteria.

In some embodiments, the disclosure relates to treating a bacterial infection such as community-acquired pneumoniae, hospital-acquired pneumoniae, skin & skin structure infections, gonococcal cervicitis, gonococcal urethritis, febrile neutropenia, osteomyelitis, endocarditis, urinary tract infections and infections caused by drug resistant bacteria such as penicillin-resistant penicillin-resistant Streptococcus pneumoniae, methicillin-resistant Staphylococcus aureus, methicillin-resistant Staphylococcus epidermidis and vancomycin-resistant enterococci, syphilis, ventilator-associated pneumonia, intra-abdominal infections, gonorrhoeae, meningitis, tetanus, or tuberculosis.

In some embodiments, the disclosure relates to treating a fungal infections such as infections caused by tinea versicolor, microsporum, trichophyton, epidermophyton, candidiasis, cryptococcosis, or aspergillosis.

In some embodiments, the disclosure relates to treating an infection caused by protozoa including, but not limited to, malaria, amoebiasis, giardiasis, toxoplasmosis, cryptosporidiosis, trichomoniasis, leishmaniasis, sleeping sickness, or dysentery.

Certain compounds disclosed herein are useful to prevent or treat an infection of a malarial parasite in a subject and/or for preventing, treating and/or alleviating complications and/or symptoms associated therewith and can then be used in the preparation of a medicament for the treatment and/or prevention of such disease. The malaria may be caused by Plasmodium falciparum, P. vivax, P. ovale, or P. malariae.

In one embodiment, the compound is administered after the subject has been exposed to the malaria parasite. In another embodiment, a compound disclosed herein is administered before the subject travels to a country where malaria is endemic.

The compounds or the above-mentioned pharmaceutical compositions may also be used in combination with one or more other therapeutically useful substances selected from the group comprising antimalarials like quinolines (e.g., quinine, chloroquine, amodiaquine, mefloquine, primaquine, tafenoquine); peroxide antimalarials (e.g., artemisinin, artemether, artesunate); pyrimethamine-sulfadoxine antimalarials (e.g., Fansidar); hydroxynaphtoquinones (e.g., atovaquone); acroline-type antimalarials (e.g., pyronaridine); and antiprotozoal agents such as ethylstibamine, hydroxystilbamidine, pentamidine, stilbamidine, quinapyramine, puromycine, propamidine, nifurtimox, melarsoprol, nimorazole, nifuroxime, aminitrozole and the like.

In an embodiment, compounds disclosed herein can be used in combination one additional drug selected from the group consisting of chloroquine, artemesin, qinghaosu, 8-aminoquinoline, amodiaquine, arteether, artemether, artemisinin, artesunate, artesunic acid, artelinic acid, atovoquone, azithromycine, biguanide, chloroquine phosphate, chlorproguanil, cycloguanil, dapsone, desbutyl halofantrine, desipramine, doxycycline, dihydrofolate reductase inhibitors, dipyridamole, halofantrine, haloperidol, hydroxychloroquine sulfate, imipramine, mefloquine, penfluridol, phospholipid inhibitors, primaquine, proguanil, pyrimethamine, pyronaridine, quinine, quinidine, quinacrineartemisinin, sulfonamides, sulfones, sulfadoxine, sulfalene, tafenoquine, tetracycline, tetrandine, triazine, salts or mixture thereof.

Compounds of the present disclosure can be administered in combination with a second agent, e.g., including, but not limited to, an antiviral agent such as a direct-acting antiviral agent, an indirect acting antiviral agent, and a host-directed antiviral agent. In various aspects, compounds of the present disclosure can be administered in combination with a second agent, including, but not limited to, immunomodulators, such as interleukin 6 (IL-6) inhibitors, corticosteroids, TNF-inhibitors, and other immune-dependent therapies; antibody therapies, such as convalescent plasma therapies, hyperimmune globulin therapies, monoclonal antibodies, polyclonal antibodies, and neutralizing antibodies; soluble guanylate cyclase stimulator, such as riociguat; cannibidiols; and vaccines. The additional therapies contemplated include biological products that are biosimilar to any biological product or therapy expressly listed herein.

In further various aspects, compounds of the present disclosure can be administered in combination with a second agent, including, but are not limited to 2,3,4,5,6-pentafluoro-N-(3-fluoro-4-methoxyphenyl) benzene sulfonamide, 3′,4′-didehydro-4′deoxy-8′-norvin-caleukoblastine, 47D11, 5-fluorouracil, abatacept, abacavir, abiraterone acetate, ABT-450 and/or ABT-267, and/or ABT-333, ABX464, abibertinib, acalabrutinib, ACE2-Fc, ACE-MAB (STI-4920, CMAB020), acetylsalicylic acid, acetaminophen, ACT-20, Actemra, Actemra/RoActemra, acyclovir, adefovir, adalimumab, adipose mesenchymal cells, AdMSCs (autologous adipose-derived stem cells), ADR-001, adrecizumab (HAM8101), ADX-629/reproxalap, AK-119, Alferon N, Allocetra (leukocyte cell based therapy), AlloStim, Allorx stem cells, AL T-100 (enamptcumab), AL T-803, altretamine, amantadine, Amnioboost, amiodarone, Ampion, ampligen, amprenavir, arbidol, asunaprevir, atazanavir, atripla, Anaferon, Anakinra, AMG-3777, anhydrovinblastine, anti-nCoV nanoviricides, aprepitant, AP-003 (AntiCovir), APL-9 (pegylated synthetic cyclic peptide), APX-115, AQCH, AR-701, ARO-COV, AS-1411, ascorbic acid, asunercept, atovaquone/azithromycin, AT-100 (rhSP-D), AT-301, AT-H201, ATI-450, ATR-002, auristatin, avdoralimab (IPH5401), axatilimab, AZD-1061, AZD-7442, alvelestat (AZD-9668), AZD-8895, azvudine, azvudine/tetrandrine, azithromycin, baloxivir, BI-201335, BI-27127, boceprevir, bardoxolone, bardoxolone methyl, baricitinib, BBT-032, bemcentinib, BGE-175, BIO-300, BIOMEDIVR, bevacizumab, bexarotene, bicalutamide, BIO-1106, BLD-2660, BLD-2736, BOLD-I 00, brequinar sodium, brilacidin, bromhexine hydrochloride, BTL-TML001, bleomycin, BMS-986253, BMS 184476, BT-086, BT-588, BXCL501, BXT-25, bucillarnine, budesonide, cidofovir, combivir, daclatasvir, cachectin, acalabrutinib, camrelizumab, camrelizumab/thymosin, captopril, CardiolRx, carrimycin, cavaltinib, comostat, camostat mesylate, canakinumab, CAP-1002, carboplatin, carmustine, CB5064 analogs, CD24Fc (recombinant fusion protein), cepharanthine, cemadotin, cenicriviroc, canthaquine, CERC-002, chlorambucil, chloropromazine, cholecalciferol, ciclesonide, cisplatin, ci-trimoxazole, CK-0802, clazakizumab, clarithromycin, CLBS-119, CM4620-IE, colchicine, CorLiCyte (umbilical cord lining stem cells), COVID-19 aptamer therapy, COVID-19 human mAb, COVID-19 neutralizing antibodies, COVID-19 siRNA therapy, COVID-HIG, COVID-EIG, spike glycoproteins, CoviGlobulin, COVI-GUARD (STI-1499), CPI-006, crizanlizumab, cryptophycin, CSL-324, CT-P59, CTAP-101, CV-15, CVL-218, cyclosporine, cell replacement therapies, cyclophosphamide, CYNK-001, cytarabine, danoprevir, darunavir, delavirdine, deleobuvir, didanosine, disoxaril, docosanol, dacarbazine, dactolisib, dactinomycin, dalargin, DAS-181, dapagliflozin, dapansurtrile, daunorubicin, decitabine, dexamethasone, DNL 758 (SAR443122, RIPKI inhibitor), dipyridamole, DMX-200, DS-2319, deupirfenidone, duvelisib, DV-890, DWRX-2003, docetaxol, dolastatin, doxetaxel, doxorubicin (adriamycin), DP-710, edoxudine, efavirenz, enfuvirtide, ensitrelvir, entecavir, EB-05, EB-201, ebastine, eculizumab, EDP-1815, efineptakin alfa, emapalumab, emtricitabine, ensifentrine, ENU-200, enoxaparin, enzalutarnide, epaspire, etanercept, etoposide, eravacycline, favipiravir, famciclovir, fomivirsen, fosamprenavir, foscarnet, fosfonet, famotidine, finasteride, fingolimod, flebogamma (IGIV31), fluvoxarnine, foalumab (NI-0401, TZLS-401), fostamatinib, flutarnide, FSD-201, FW1022, FT516, ganciclovir, GS-5882, GS-9190, GS-9451, Gamunex (IGIV-C), ganetespib, GC-376, Giapreza, GLS-1200, garadacimab, GC-5131A (hyperimmune globulin), GIGA-2050 rCIG), gimsilumab, GNS561, GP1681, GSK-2586881/APN-1, GSK-4182136, GTB-3550 (Trike 161533), haNK:CD-16, HB-adMSCs, HFB30132A, HLCM-051, heparin, hydrocortisone, hydroxyurea, ibuprofen, ibudilasst (MN-166), icosapent ethyl, IC14, IDB-003, IFX-1/BDB-1, IgY-110, IMM101 IMS001, IMS002, ibacitabine, imunovir, idoxuridine, imiquimod, indinavir, inosine, ifosfarnide, imatinib, infliximab, INM-005, interferon alfa, interferon alfa 1B, interferon alfa 2B, interferon beta IA, interferon beta 1B, interleukin-6, interleukin-7, isoquercetin, itanapraced (CHF-5074), itolizumab, ivermectin, IVIG, JS012 (monoclonal antibody, LY-CoV016), jaktinib, kagocel, KB109, K-NK-1D101, KTH-222, lamivudine, laninamivir octanoate, ledisavir, lopinavir or lopinavir/ritonavir, loviride, lactoferrin, LAM-002A (apilimod dimesylate), lanadelumab, lamellasome, LB-1148, larazotide, leflunornide, lenzilumab, leronlimab (monoclonal antibody), levilimab (BCD-089), levarnisole, liarozole, linagliptin, lipocurc, losartan, livilimab, lomustine (CCNU), lonidarnine, losmapimod, lostartan, LY-CoV555 (LY-3819253), LY-3127804, molnupiravir, moroxydine, methisazone, mannitol, maraviroc, mastinib, mavrilimumab, MDV3100, mechlorethamine, MEDI-3506, melatonin, melphalan, meplazumab, merimepodib, Mesenchymal stem cells (MSCs), mesencure (cell replacement), metablok (anti-inflammatory), metformin, methotrexate, methylprednisolone, mitomycin, mivobulin isethionate, mosedipimod (EC-18), MP-0420, MP-0423, MRx4DP0004, N-acetylcysteine, N,N-dimethyl-L-valyl-L-valyl-N-methyl-L-valyl-L-prolyl-1-Lproline-t-butylamide, nelfinavir, nevirapine, nexavir, namilumab (IZN-101), nangibotide, narsoplimab, nebulized domase alfa, NED-260, Niagen (nicotinamide riboside; Vitamin B3), NK cell therapy, niclosamide, nilutamide, nintedanib, nitric oxide, nivolumab, NL-CVXI, NLP-21, NP-02, N-120 (ifenprodil), novaferon, NT-17 (efineptakin alfa), NTR-441, oseltamivir, ° Ctagam, olokizumab, omeprazole, onapristone, opaganib, OP-101, OT-101 (trabedersen), otilimab, ozanimod, paxlovid, penciclovir, peramivir, pirodavir, pleconaril, pocapavir, podophyllotoxin, PPI-668, PSI-7977, PSI-938, paclitaxel, pacritinib, panaphix, pamrevlumab, paracetamol, PAXLOVID™, PB1046, PTC299, pegylated interferon alpha, pegylated interferon alpha 2b, pegylated interferon lambda, pembrolizumab, PL-8177, pirfenidone, plitidepsin (aplidin), PneumoBlast, polyoxidonium, prazosin, prednimustine, prednisolone, prednisone, pritumumab, procarbazine, prolastin, PTC-299, pyronaridine/artesunate, radotinib, RAPA-501, raltegravir, remdesivir, ribavirin, rimantadine, ritonavir, ravulizumab, razuprotafib, interferon beta 1 agonists, RECC327, REGN-COV2 (antibody cocktail), reparixin, rintatolimod (ampligen), RLF-100 (aviptadil), RLS-0071, STI-5656 (abivertinib), Rhu-pGSN (gelsolin), rhizoxin, RPR109881, RoActemra, RUCONEST (conestat alfa), ruxolitinib, SAB-185, SAR443122, sarilumab, SARS-CoV-2 antibodies, SARS-Co V-2 monoclonal antibodies, SARS-Co V-2 poly clonal antibodies, SARS-Co V-2 neutrali pyramidine, samatasvir, saquinavir, simeprevir, sofosbuvir, stavudine, SCTA01, Leukine (sargramostim), selenexor, sevoflurane, sertenef, siltuximab, sildenafil citate, silymarin, simvastatin, sirolimus, sirukumab, SIW A-318, solnatide, SNG-001, ST-266, stem cell educator therapy, STI-1499, STI-2020dna (COVI-MAB), STI-4398 (Covidtrap), stramustine phosphate, streptozocin, T cell therapies (TargNaturTa), TAK-671, TAK-888, TATX-36, TATX-99, TCB-007, TJ003234/TJM-2, TP508, TRV027, TD-0903, TLC19, TMC-435, telaprevir, tenofovir, tenofovir disoproxil, tipranavir, trifluridine, trizivir, tromantadine, truvada, tekruma, tafenoquine, tamoxifen, tasonermin, taxanes, taxol, tetradrine, thalidomide, thimerosal, thymalfasin, tinzaparin, tocilizumab, tofacitinib, toremifene, tradipitant, tranexamic acid, trans sodium crocetinate (TSC), tramadol, tretinoin, TXA127 (antiotensin-(1-7) peptide), TY027, TZLS-501, UNI-911, ulinastatin, upamostat, valaciclovir, valganciclovir, vapendavir, vicriviroc, vidarabine, viramidine zalcitabine, vafidemstat, valsartan, icosapent ethyl, vazegepant, VBI-S, VERU-111, VHH72-Fc, vinblastine, vincristine, vindesine sulfate, vinflunine, VIR-2703 ALN-COV), VIR-7831, VIR-7832, Vitamin C, Vitamin D, VX-135, VX-222, XAV-19, Xpro-1595, XRx-101, zanamivir, zidovudine, zing antibodies, zanubrutinib, zilucoplan, zinc; and/or a such as a compound disclosed in any of US 2010/0144608, U.S. 61/339,964, US 2011/0312973, WO 2009/039127, US 2010/0317568, 2012/151158, US 2012/0172290, WO 2012/092411, WO 2012/087833, WO 2012/083170, WO 2009/039135, US 2012/0115918, WO 2012/051361, WO 2012/009699, WO 2011/156337, US 2011/0207699, WO 2010/075376, U.S. Pat. No. 7,910,595, WO 2010/120935, WO 2010/111437, WO 2010/111436, US 2010/0168384 or US 2004/0167123; and salts and/or prodrugs thereof, as well as combinations thereof, or a prodrug of the foregoing as appropriate; and/or a physiological or pharmaceutical salt thereof, as appropriate; and/or combinations thereof.

In a particular embodiment, compounds of the present disclosure can be administered in combination with a second antiviral including, but not limited to, abacavir, acyclovir, acyclovir, adefovir, amantadine, amprenavir, ampligen, arbidol, atazanavir, atripla, baloxivir, BI-201335, BI-27127, boceprevir, cidofovir, combivir, daclatasvir, danoprevir, darunavir, delavirdine, deleobuvir, didanosine, disoxaril, docosanol, edoxudine, efavirenz, emtricitabine, enfuvirtide, ensitrelvir, entecavir, favipiravir, famciclovir, fomivirsen, fosamprenavir, foscarnet, fosfonet, ganciclovir, GS-5882, GS-9190, GS-9451, ibacitabine, imunovir, idoxuridine, imiquimod, indinavir, inosine, interferon type III, interferon type II, interferon type I, lamivudine, laninamivir ° Ctanoate, ledisavir, lopinavir or lopinavir/ritonavir, loviride, maraviroc, molnupiravir, moroxydine, methisazone, nelfinavir, nevirapine, nexavir, oseltamivir, paxlovid, peginterferon alfa-2a, penciclovir, peramivir, pirodavir, pleconaril, pocapavir, podophyllotoxin, PPI-668, PSI-7977, PSI-938, raltegravir, remdesivir, ribavirin, rimantadine, ritonavir, pyramidine, samatasvir, saquinavir, simeprevir, sofosbuvir, stavudine, TMC-435, telaprevir, tenofovir, tenofovir disoproxil, tipranavir, trifluridine, trizivir, tromantadine, truvada, valaciclovir, valganciclovir, vapendavir, vicriviroc, vidarabine, viramidine zalcitabine, zanamivir, zidovudine, a compound disclosed in any of US 2010/0144608, U.S. 61/339,964, US 2011/0312973, WO 2009/039127, US 2010/0317568, 2012/151158, US 2012/0172290, WO 2012/092411, WO 2012/087833, WO 2012/083170, WO 2009/039135, US 2012/0115918, WO 2012/051361, WO 2012/009699, WO 2011/156337, US 2011/0207699, WO 2010/075376, U.S. Pat. No. 7,910,595, WO 2010/120935, WO 2010/111437, WO 2010/111436, US 2010/0168384 or US 2004/0167123, VX-135, VX-222, and salts and/or prodrugs thereof, as well as combinations thereof.

In a particular embodiment, compounds of the present disclosure can be administered in combination with a second antiviral agent such as a host-directed antiviral agent, including, but not limited to, IFN-α-2a (including pegylated forms thereof, and used alone or in combination with ribavirin), IFN-α-2b (including pegylated forms thereof, and used alone or in combination with ribavirin), IFN-α-N3, IFN-β-1a (including pegylated forms thereof, and used alone or in combination with ribavirin), IFN-β-1b (including pegylated forms thereof, and used alone or in combination with ribavirin), podofilox, interferon alfacon-1, imiquimod, one or more sincatechin, maraviroc, DAS181, R448 (cabozantinib), eztimibe, obatoclax, glycyrrhizin, concanamycin, daptomycin, LJ001, thapsigargin, dynasore, MLS000394177, MLS000733230, MLS000730532, bisindolylmaleimide I, calphostin C, chelerythrine, enzastaurin, staurosporine, fattiviracin, rintatolimod, GS9620, R06864018, R07020531, AL-034, imiquimod, GS9688, CL097, PF-04878691 or 852A, CPG10101, IMO-2125, SD-101, inarigivir (SB 9200), quercetin, A23187, phorbol myristate acetate, CI1033, a SIP agonist, a PPAR agonist, and combinations thereof.

In a particular embodiment, a second agent, e.g., an antiviral agent, as disclosed herein is administered together with one of the following compounds:

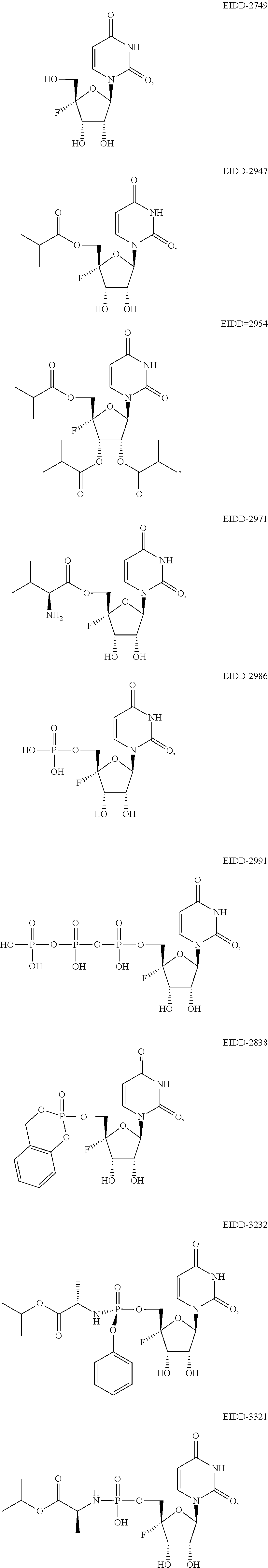

and combinations thereof.

In a particular embodiment, a second agent, e.g., an antiviral agent, as disclosed herein is administered together with one of the following compounds:

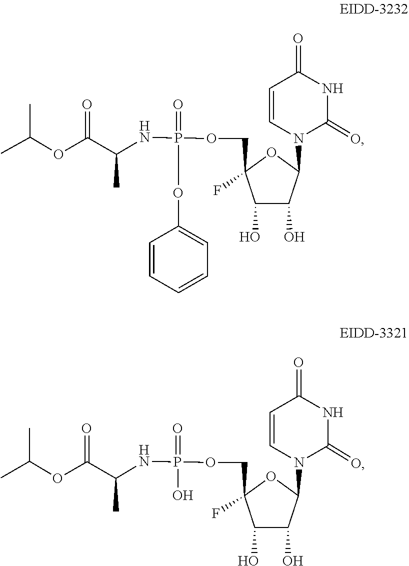 [SMILES: CC(C)OC(=O)[C@H](C)NP(=O)(OC[C@@]1(F)O[C@@H](n2ccc(=O)[nH]c2=O)[C@H](O)[C@@H]1O)Oc1ccccc1 ; CC(C)OC(=O)[C@H](C)NP(=O)(O)OC[C@@]1(F)O[C@@H](n2ccc(=O)[nH]c2=O)[C@H](O)[C@@H]1O]

and a combination thereof.

In a particular embodiment, a second agent, e.g., an antiviral agent, as disclosed herein is administered together with one of the following compounds:

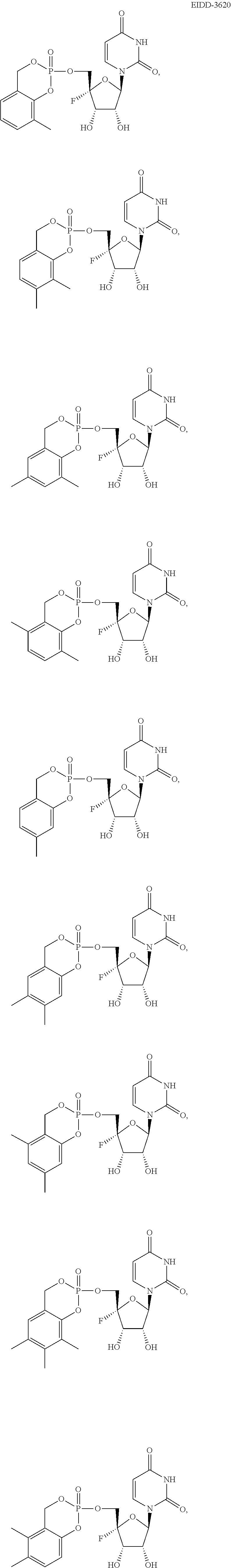

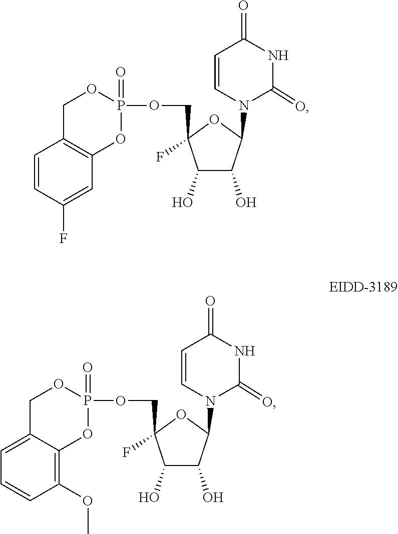 [SMILES: O=c1ccn([C@@H]2O[C@](F)(COP3(=O)OCc4ccc(F)cc4O3)[C@@H](O)[C@H]2O)c(=O)[nH]1 ; COc1cccc2c1OP(=O)(OC[C@@]1(F)O[C@@H](n3ccc(=O)[nH]c3=O)[C@H](O)[C@@H]1O)OC2]

and combinations of the foregoing.

In a particular embodiment, a second agent, e.g., an antiviral agent, as disclosed herein is administered together with one of the following compounds:

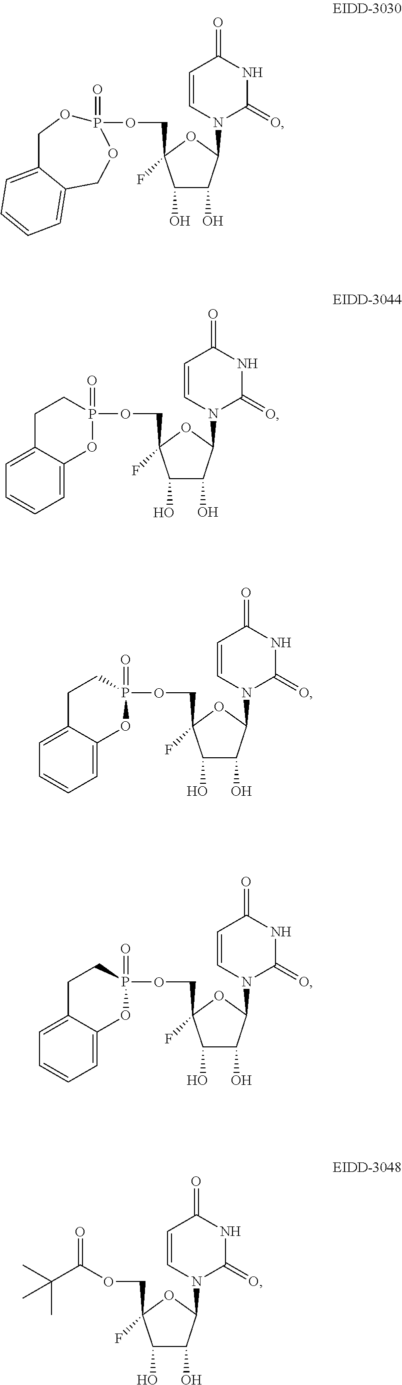 [SMILES: O=c1ccn([C@@H]2O[C@](F)(COP3(=O)OCc4ccccc4CO3)[C@@H](O)[C@H]2O)c(=O)[nH]1 ; O=c1ccn([C@@H]2O[C@](F)(COP3(=O)CCc4ccccc4O3)[C@@H](O)[C@H]2O)c(=O)[nH]1 ; O=c1ccn([C@@H]2O[C@](F)(CO[P@@]3(=O)CCc4ccccc4O3)[C@@H](O)[C@H]2O)c(=O)[nH]1 ; O=c1ccn([C@@H]2O[C@](F)(CO[P@]3(=O)CCc4ccccc4O3)[C@@H](O)[C@H]2O)c(=O)[nH]1 ; CC(C)(C)C(=O)OC[C@@]1(F)O[C@@H](n2ccc(=O)[nH]c2=O)[C@H](O)[C@@H]1O]

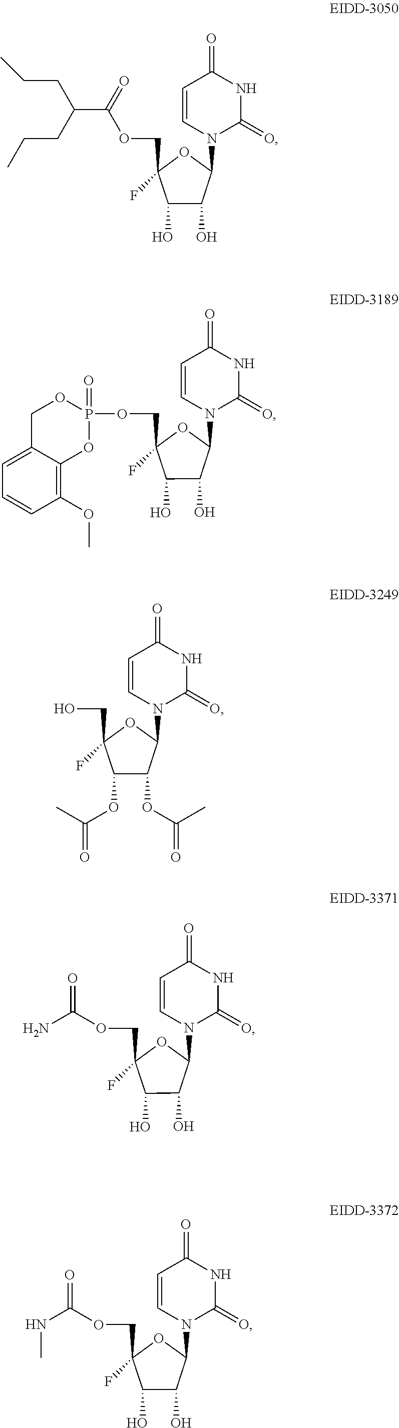 [SMILES: CCCC(CCC)C(=O)OC[C@@]1(F)O[C@@H](n2ccc(=O)[nH]c2=O)[C@H](O)[C@@H]1O ; COc1cccc2c1OP(=O)(OC[C@@]1(F)O[C@@H](n3ccc(=O)[nH]c3=O)[C@H](O)[C@@H]1O)OC2 ; CC(=O)O[C@H]1[C@H](n2ccc(=O)[nH]c2=O)O[C@](F)(CO)[C@H]1OC(C)=O ; NC(=O)OC[C@@]1(F)O[C@@H](n2ccc(=O)[nH]c2=O)[C@H](O)[C@@H]1O ; CNC(=O)OC[C@@]1(F)O[C@@H](n2ccc(=O)[nH]c2=O)[C@H](O)[C@@H]1O]

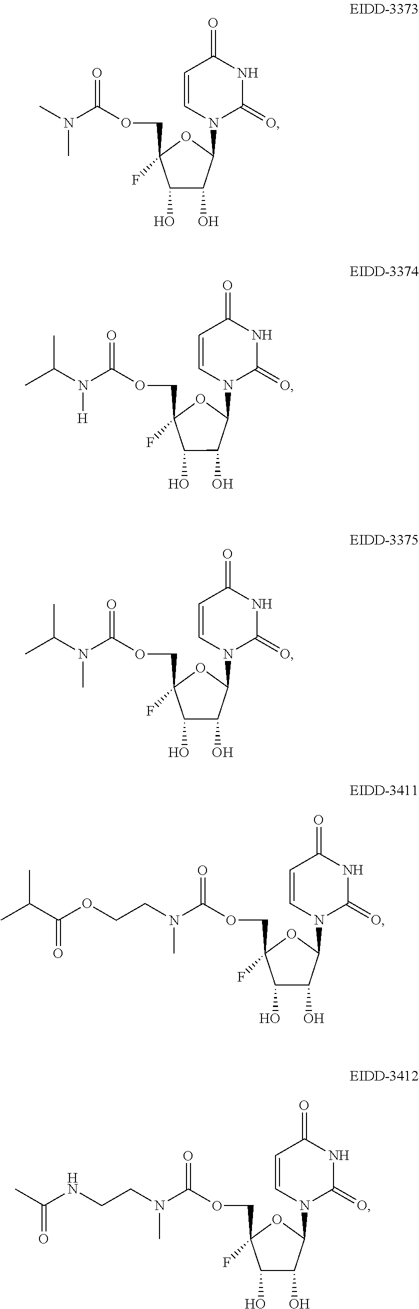 [SMILES: CN(C)C(=O)OC[C@@]1(F)O[C@@H](n2ccc(=O)[nH]c2=O)[C@H](O)[C@@H]1O ; CC(C)NC(=O)OC[C@@]1(F)O[C@@H](n2ccc(=O)[nH]c2=O)[C@H](O)[C@@H]1O ; CC(C)N(C)C(=O)OC[C@@]1(F)O[C@@H](n2ccc(=O)[nH]c2=O)[C@H](O)[C@@H]1O ; CC(C)C(=O)OCCN(C)C(=O)OC[C@@]1(F)O[C@@H](n2ccc(=O)[nH]c2=O)[C@H](O)[C@@H]1O ; CC(=O)NCCN(C)C(=O)OC[C@@]1(F)O[C@@H](n2ccc(=O)[nH]c2=O)[C@H](O)[C@@H]1O]

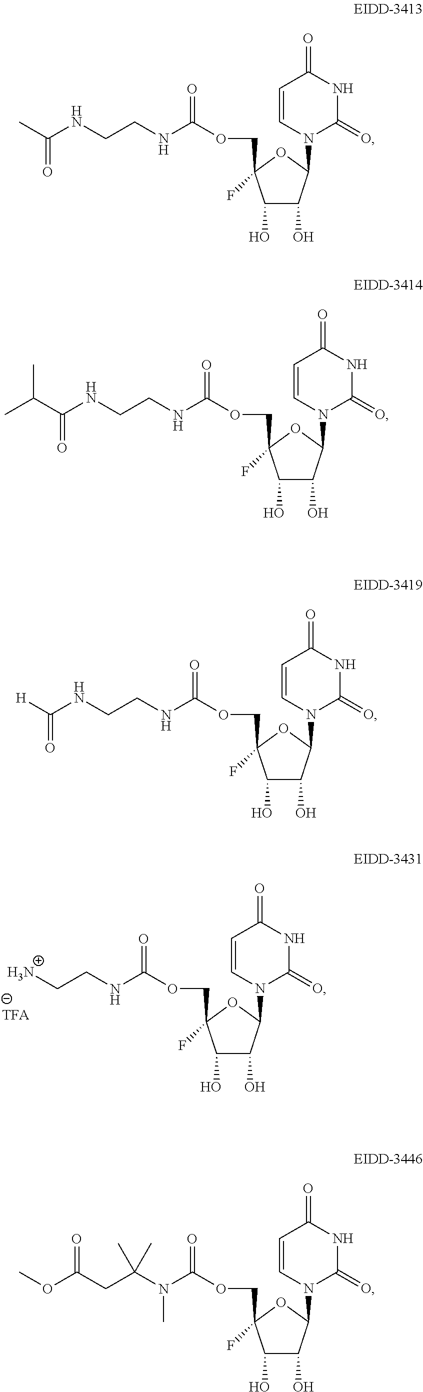 [SMILES: CC(=O)NCCNC(=O)OC[C@@]1(F)O[C@@H](n2ccc(=O)[nH]c2=O)[C@H](O)[C@@H]1O ; CC(C)C(=O)NCCNC(=O)OC[C@@]1(F)O[C@@H](n2ccc(=O)[nH]c2=O)[C@H](O)[C@@H]1O ; O=CNCCNC(=O)OC[C@@]1(F)O[C@@H](n2ccc(=O)[nH]c2=O)[C@H](O)[C@@H]1O ; [NH3+]CCNC(=O)OC[C@@]1(F)O[C@@H](n2ccc(=O)[nH]c2=O)[C@H](O)[C@@H]1O ; COC(=O)CC(C)(C)N(C)C(=O)OC[C@@]1(F)O[C@@H](n2ccc(=O)[nH]c2=O)[C@H](O)[C@@H]1O]

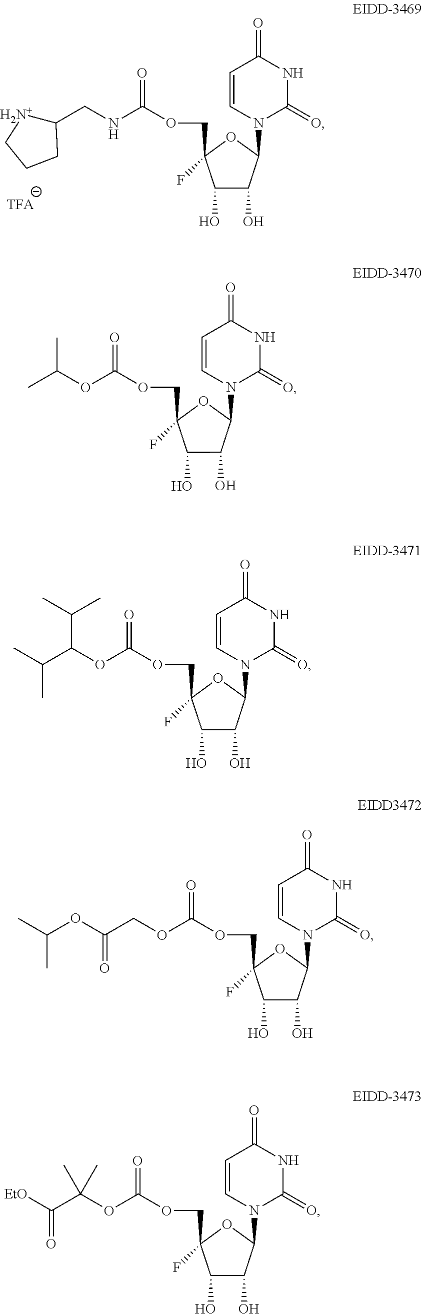 [SMILES: O=C(NCC1CCC[NH2+]1)OC[C@@]1(F)O[C@@H](n2ccc(=O)[nH]c2=O)[C@H](O)[C@@H]1O ; CC(C)OC(=O)OC[C@@]1(F)O[C@@H](n2ccc(=O)[nH]c2=O)[C@H](O)[C@@H]1O ; CC(C)C(OC(=O)OC[C@@]1(F)O[C@@H](n2ccc(=O)[nH]c2=O)[C@H](O)[C@@H]1O)C(C)C ; CC(C)OC(=O)COC(=O)OC[C@@]1(F)O[C@@H](n2ccc(=O)[nH]c2=O)[C@H](O)[C@@H]1O ; CCOC(=O)C(C)(C)OC(=O)OC[C@@]1(F)O[C@@H](n2ccc(=O)[nH]c2=O)[C@H](O)[C@@H]1O]

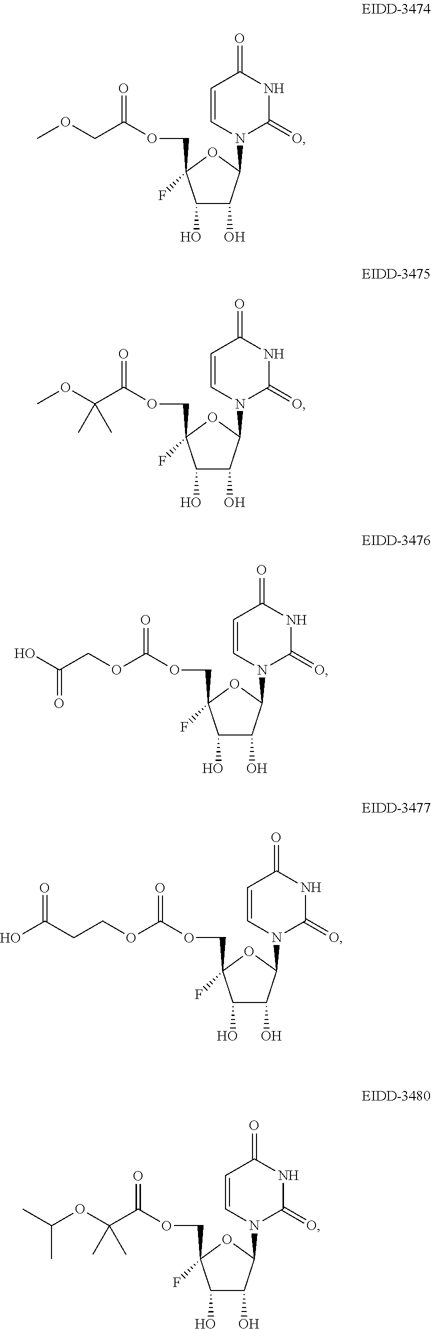 [SMILES: COCC(=O)OC[C@@]1(F)O[C@@H](n2ccc(=O)[nH]c2=O)[C@H](O)[C@@H]1O ; COC(C)(C)C(=O)OC[C@@]1(F)O[C@@H](n2ccc(=O)[nH]c2=O)[C@H](O)[C@@H]1O ; O=C(O)COC(=O)OC[C@@]1(F)O[C@@H](n2ccc(=O)[nH]c2=O)[C@H](O)[C@@H]1O ; O=C(O)CCOC(=O)OC[C@@]1(F)O[C@@H](n2ccc(=O)[nH]c2=O)[C@H](O)[C@@H]1O ; CC(C)OC(C)(C)C(=O)OC[C@@]1(F)O[C@@H](n2ccc(=O)[nH]c2=O)[C@H](O)[C@@H]1O]

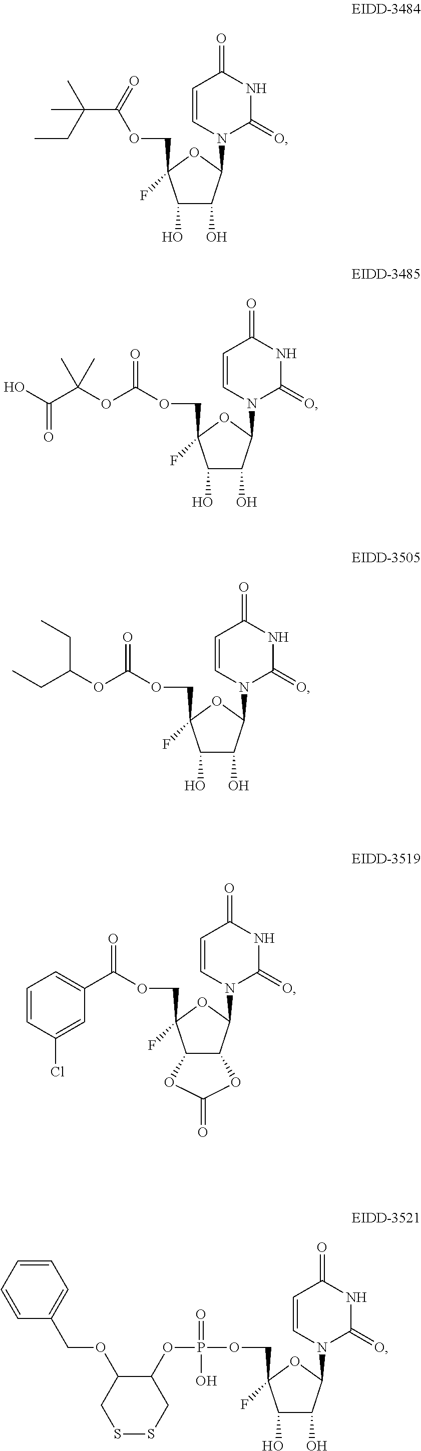 [SMILES: CCC(C)(C)C(=O)OC[C@@]1(F)O[C@@H](n2ccc(=O)[nH]c2=O)[C@H](O)[C@@H]1O ; CC(C)(OC(=O)OC[C@@]1(F)O[C@@H](n2ccc(=O)[nH]c2=O)[C@H](O)[C@@H]1O)C(=O)O ; CCC(CC)OC(=O)OC[C@@]1(F)O[C@@H](n2ccc(=O)[nH]c2=O)[C@H](O)[C@@H]1O ; O=C1O[C@H]2[C@H](n3ccc(=O)[nH]c3=O)O[C@](F)(COC(=O)c3cccc(Cl)c3)[C@H]2O1 ; O=c1ccn([C@@H]2O[C@](F)(COP(=O)(O)OC3CSSCC3OCc3ccccc3)[C@@H](O)[C@H]2O)c(=O)[nH]1]

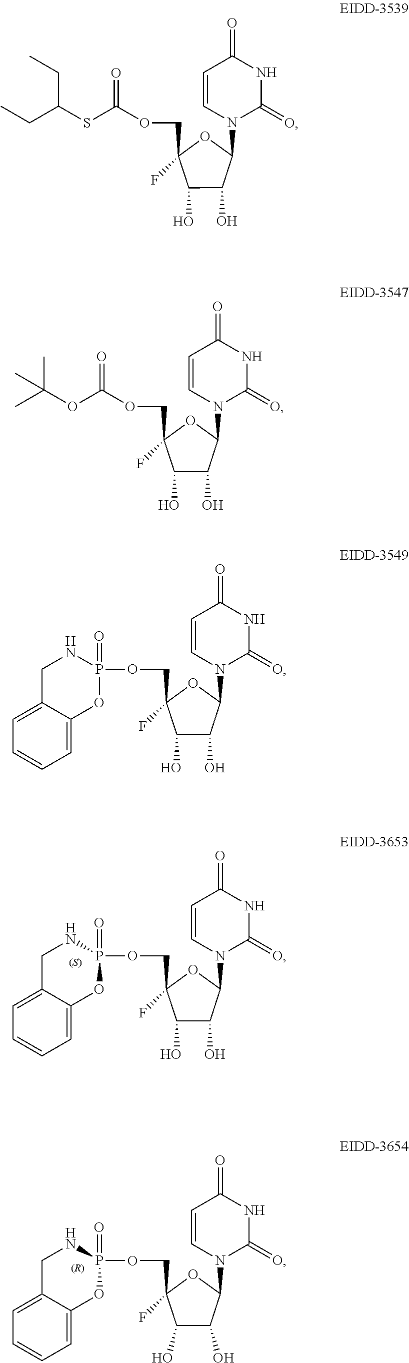

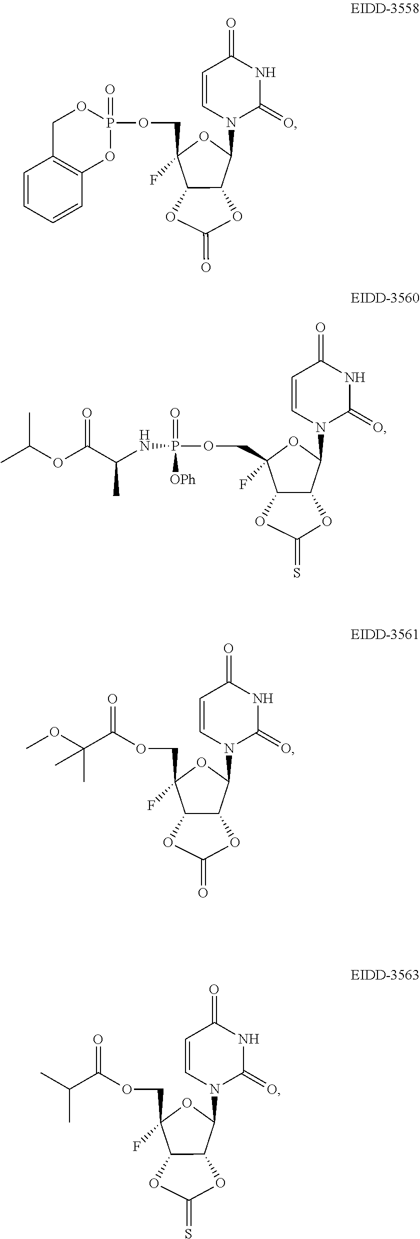

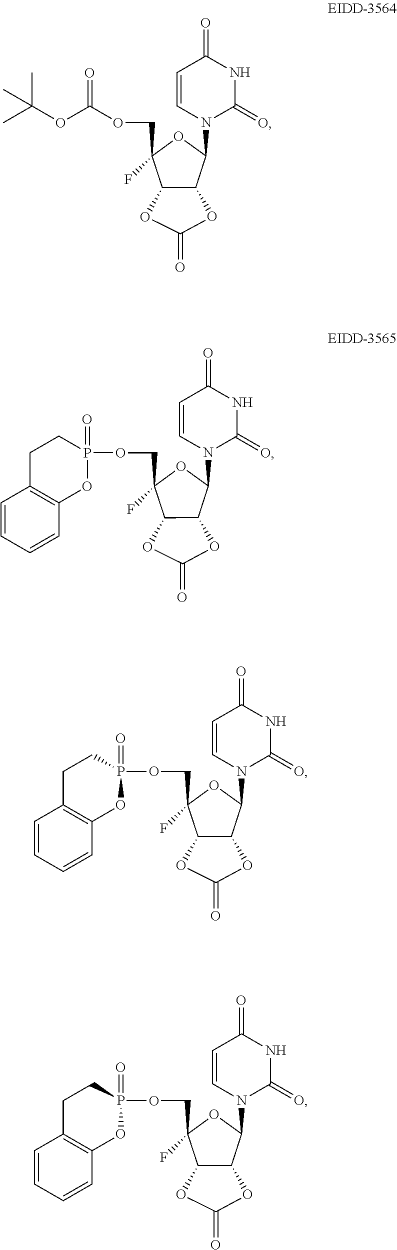 [SMILES: CC(C)(C)OC(=O)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COP3(=O)CCc4ccccc4O3)[C@H]2O1 ; O=C1O[C@H]2[C@H](n3ccc(=O)[nH]c3=O)O[C@](F)(CO[P@@]3(=O)CCc4ccccc4O3)[C@H]2O1 ; O=C1O[C@H]2[C@H](n3ccc(=O)[nH]c3=O)O[C@](F)(CO[P@]3(=O)CCc4ccccc4O3)[C@H]2O1]

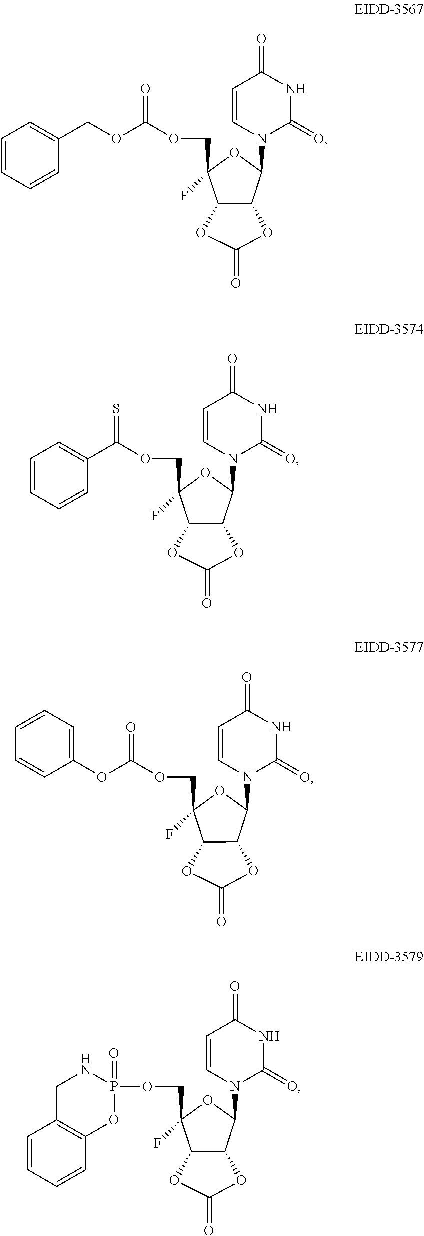 [SMILES: O=C(OCc1ccccc1)OC[C@@]1(F)O[C@@H](n2ccc(=O)[nH]c2=O)[C@@H]2OC(=O)O[C@@H]21 ; O=C1O[C@H]2[C@H](n3ccc(=O)[nH]c3=O)O[C@](F)(COC(=S)c3ccccc3)[C@H]2O1 ; O=C(OC[C@@]1(F)O[C@@H](n2ccc(=O)[nH]c2=O)[C@@H]2OC(=O)O[C@@H]21)Oc1ccccc1 ; O=C1O[C@H]2[C@H](n3ccc(=O)[nH]c3=O)O[C@](F)(COP3(=O)NCc4ccccc4O3)[C@H]2O1]

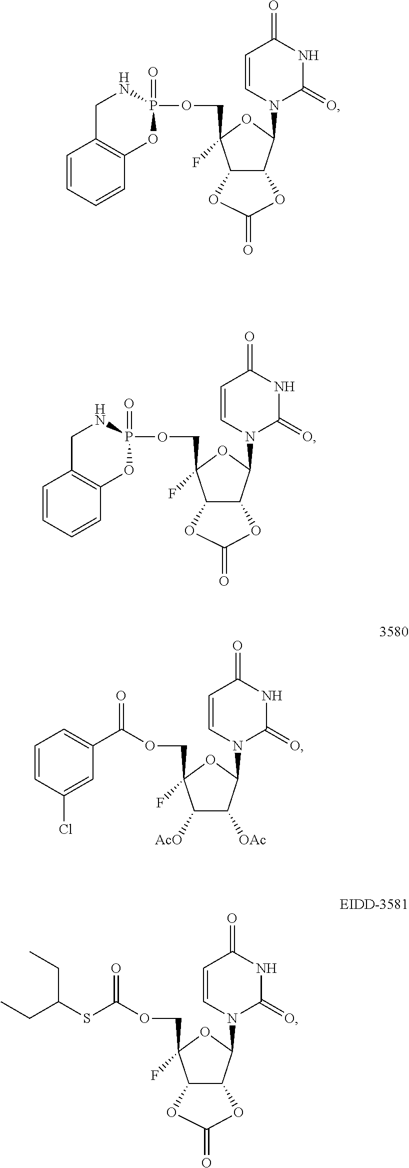 [SMILES: O=C1O[C@H]2[C@H](n3ccc(=O)[nH]c3=O)O[C@](F)(CO[P@@]3(=O)NCc4ccccc4O3)[C@H]2O1 ; O=C1O[C@H]2[C@H](n3ccc(=O)[nH]c3=O)O[C@](F)(CO[P@]3(=O)NCc4ccccc4O3)[C@H]2O1 ; CC(=O)O[C@H]1[C@H](n2ccc(=O)[nH]c2=O)O[C@](F)(COC(=O)c2cccc(Cl)c2)[C@H]1OC(C)=O ; CCC(CC)SC(=O)OC[C@@]1(F)O[C@@H](n2ccc(=O)[nH]c2=O)[C@@H]2OC(=O)O[C@@H]21]

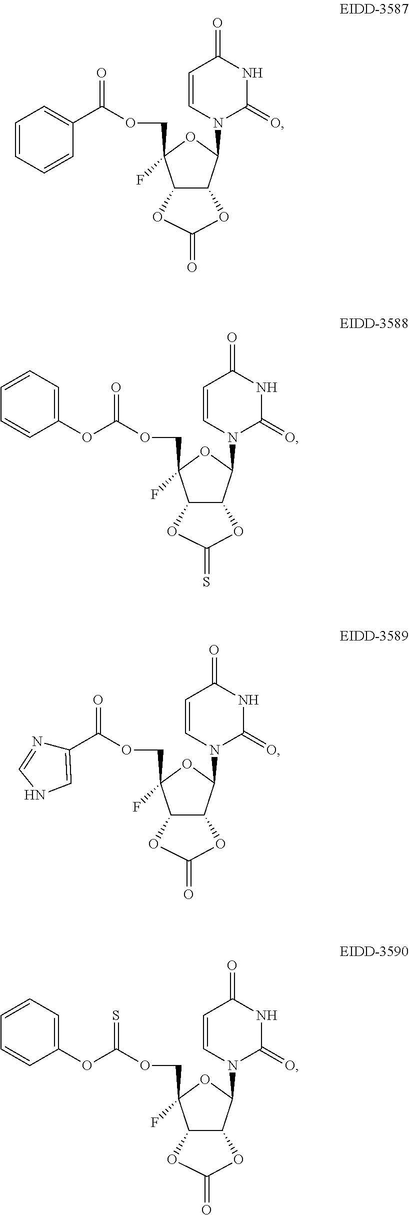 [SMILES: O=C1O[C@H]2[C@H](n3ccc(=O)[nH]c3=O)O[C@](F)(COC(=O)c3ccccc3)[C@H]2O1 ; O=C(OC[C@@]1(F)O[C@@H](n2ccc(=O)[nH]c2=O)[C@@H]2OC(=S)O[C@@H]21)Oc1ccccc1 ; O=C1O[C@H]2[C@H](n3ccc(=O)[nH]c3=O)O[C@](F)(COC(=O)c3c[nH]cn3)[C@H]2O1 ; O=C1O[C@H]2[C@H](n3ccc(=O)[nH]c3=O)O[C@](F)(COC(=S)Oc3ccccc3)[C@H]2O1]

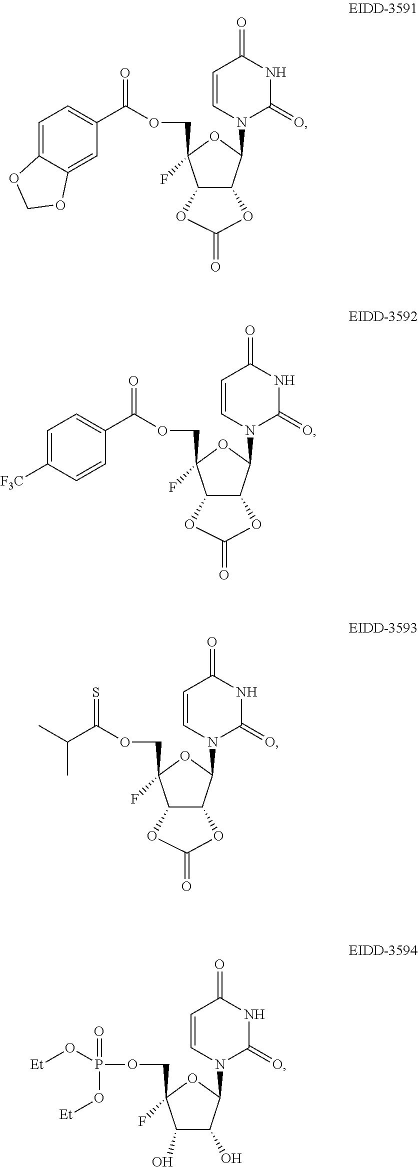 [SMILES: O=C1O[C@H]2[C@H](n3ccc(=O)[nH]c3=O)O[C@](F)(COC(=O)c3ccc4c(c3)OCO4)[C@H]2O1 ; O=C1O[C@H]2[C@H](n3ccc(=O)[nH]c3=O)O[C@](F)(COC(=O)c3ccc(C(F)(F)F)cc3)[C@H]2O1 ; CC(C)C(=S)OC[C@@]1(F)O[C@@H](n2ccc(=O)[nH]c2=O)[C@@H]2OC(=O)O[C@@H]21 ; CCOP(=O)(OCC)OC[C@@]1(F)O[C@@H](n2ccc(=O)[nH]c2=O)[C@H](O)[C@@H]1O]

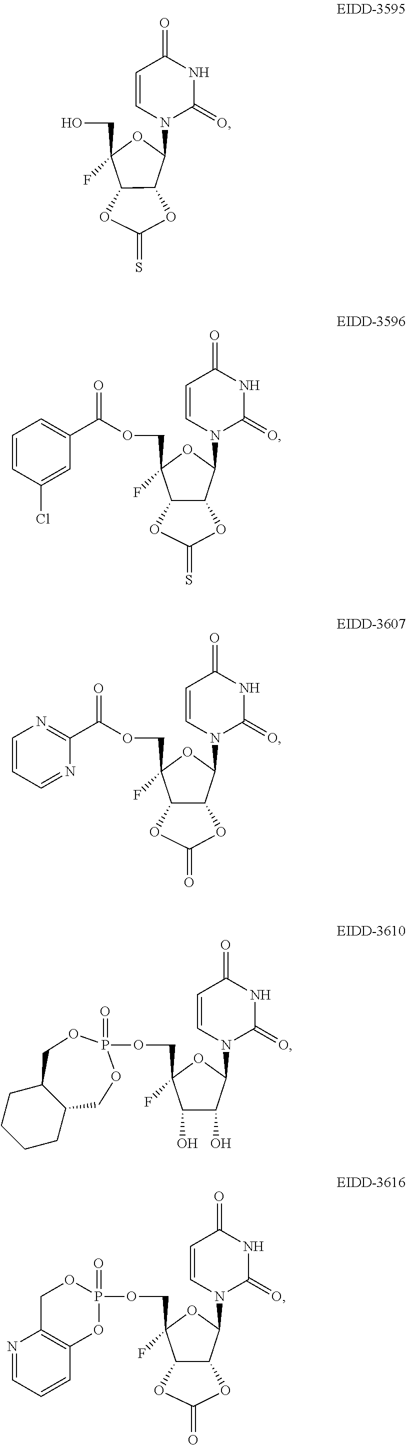 [SMILES: O=c1ccn([C@@H]2O[C@](F)(CO)[C@H]3OC(=S)O[C@@H]23)c(=O)[nH]1 ; O=C(OC[C@@]1(F)O[C@@H](n2ccc(=O)[nH]c2=O)[C@@H]2OC(=S)O[C@@H]21)c1cccc(Cl)c1 ; O=C1O[C@H]2[C@H](n3ccc(=O)[nH]c3=O)O[C@](F)(COC(=O)c3ncccn3)[C@H]2O1 ; O=c1ccn([C@@H]2O[C@](F)(COP3(=O)OC[C@@H]4CCCC[C@H]4CO3)[C@@H](O)[C@H]2O)c(=O)[nH]1 ; O=C1O[C@H]2[C@H](n3ccc(=O)[nH]c3=O)O[C@](F)(COP3(=O)OCc4ncccc4O3)[C@H]2O1]

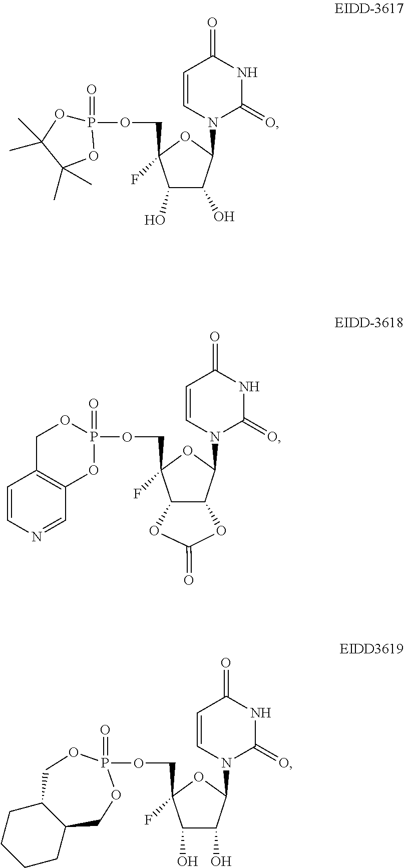 [SMILES: CC1(C)OP(=O)(OC[C@@]2(F)O[C@@H](n3ccc(=O)[nH]c3=O)[C@H](O)[C@@H]2O)OC1(C)C ; O=C1O[C@H]2[C@H](n3ccc(=O)[nH]c3=O)O[C@](F)(COP3(=O)OCc4ccncc4O3)[C@H]2O1 ; O=c1ccn([C@@H]2O[C@](F)(COP3(=O)OC[C@H]4CCCC[C@@H]4CO3)[C@@H](O)[C@H]2O)c(=O)[nH]1]

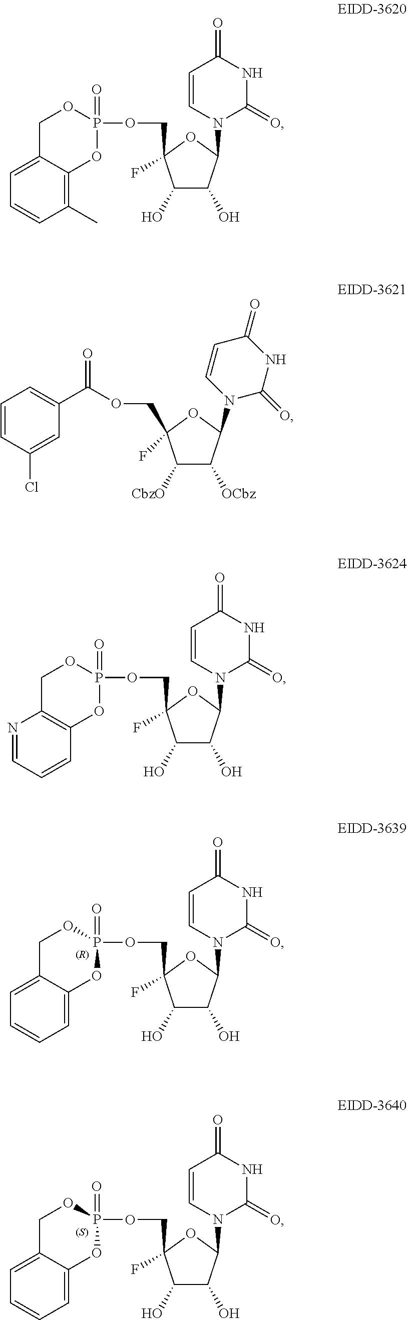

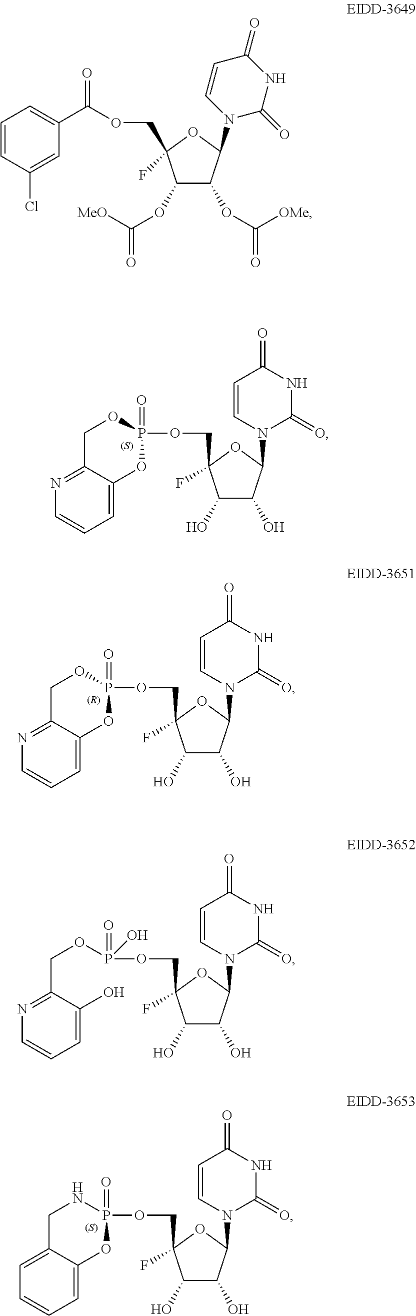

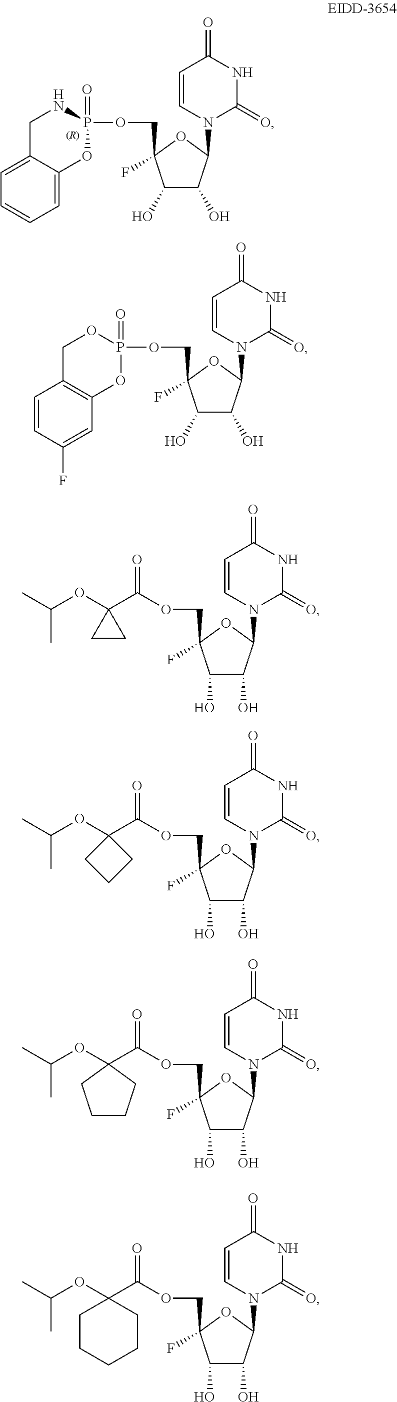

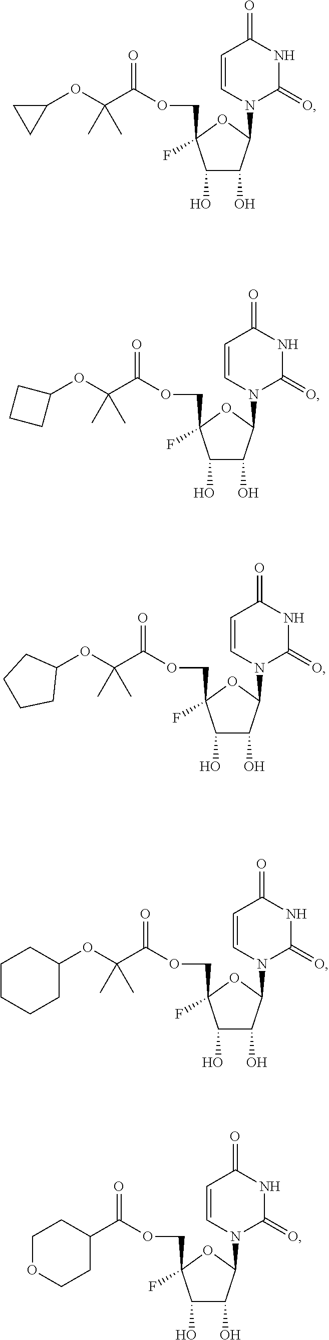 [SMILES: CC(C)(OC1CC1)C(=O)OC[C@@]1(F)O[C@@H](n2ccc(=O)[nH]c2=O)[C@H](O)[C@@H]1O ; CC(C)(OC1CCC1)C(=O)OC[C@@]1(F)O[C@@H](n2ccc(=O)[nH]c2=O)[C@H](O)[C@@H]1O ; CC(C)(OC1CCCC1)C(=O)OC[C@@]1(F)O[C@@H](n2ccc(=O)[nH]c2=O)[C@H](O)[C@@H]1O ; CC(C)(OC1CCCCC1)C(=O)OC[C@@]1(F)O[C@@H](n2ccc(=O)[nH]c2=O)[C@H](O)[C@@H]1O ; O=C(OC[C@@]1(F)O[C@@H](n2ccc(=O)[nH]c2=O)[C@H](O)[C@@H]1O)C1CCOCC1]

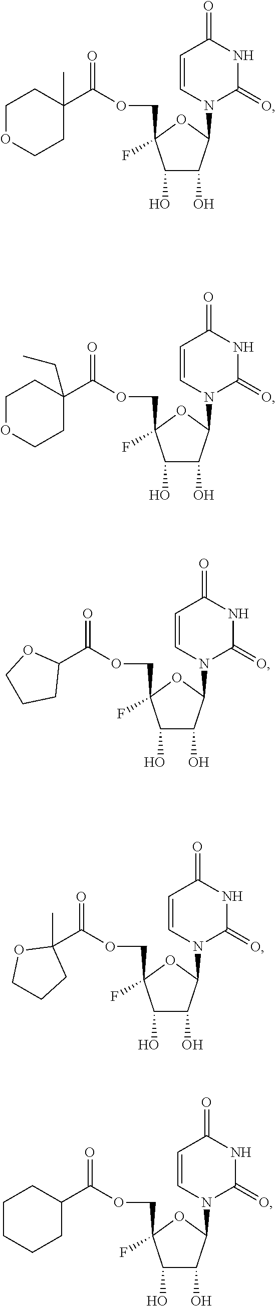 [SMILES: CC1(C(=O)OC[C@@]2(F)O[C@@H](n3ccc(=O)[nH]c3=O)[C@H](O)[C@@H]2O)CCOCC1 ; CCC1(C(=O)OC[C@@]2(F)O[C@@H](n3ccc(=O)[nH]c3=O)[C@H](O)[C@@H]2O)CCOCC1 ; O=C(OC[C@@]1(F)O[C@@H](n2ccc(=O)[nH]c2=O)[C@H](O)[C@@H]1O)C1CCCO1 ; CC1(C(=O)OC[C@@]2(F)O[C@@H](n3ccc(=O)[nH]c3=O)[C@H](O)[C@@H]2O)CCCO1 ; O=C(OC[C@@]1(F)O[C@@H](n2ccc(=O)[nH]c2=O)[C@H](O)[C@@H]1O)C1CCCCC1]

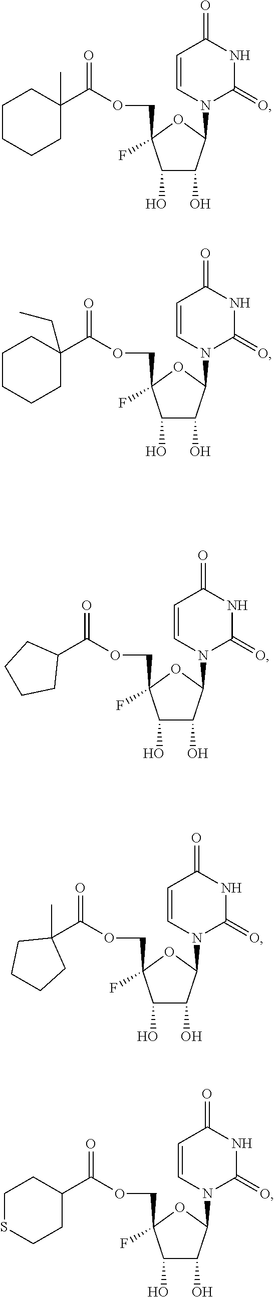 [SMILES: CC1(C(=O)OC[C@@]2(F)O[C@@H](n3ccc(=O)[nH]c3=O)[C@H](O)[C@@H]2O)CCCCC1 ; CCC1(C(=O)OC[C@@]2(F)O[C@@H](n3ccc(=O)[nH]c3=O)[C@H](O)[C@@H]2O)CCCCC1 ; O=C(OC[C@@]1(F)O[C@@H](n2ccc(=O)[nH]c2=O)[C@H](O)[C@@H]1O)C1CCCC1 ; CC1(C(=O)OC[C@@]2(F)O[C@@H](n3ccc(=O)[nH]c3=O)[C@H](O)[C@@H]2O)CCCC1 ; O=C(OC[C@@]1(F)O[C@@H](n2ccc(=O)[nH]c2=O)[C@H](O)[C@@H]1O)C1CCSCC1]

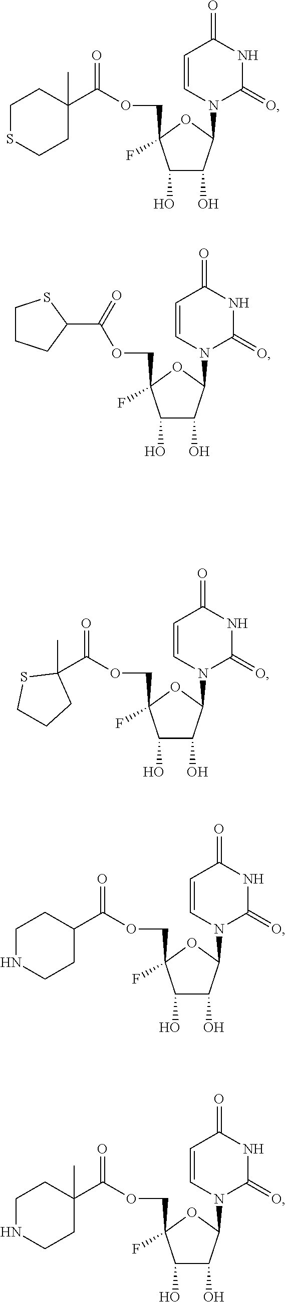 [SMILES: CC1(C(=O)OC[C@@]2(F)O[C@@H](n3ccc(=O)[nH]c3=O)[C@H](O)[C@@H]2O)CCSCC1 ; O=C(OC[C@@]1(F)O[C@@H](n2ccc(=O)[nH]c2=O)[C@H](O)[C@@H]1O)C1CCCS1 ; CC1(C(=O)OC[C@@]2(F)O[C@@H](n3ccc(=O)[nH]c3=O)[C@H](O)[C@@H]2O)CCCS1 ; O=C(OC[C@@]1(F)O[C@@H](n2ccc(=O)[nH]c2=O)[C@H](O)[C@@H]1O)C1CCNCC1 ; CC1(C(=O)OC[C@@]2(F)O[C@@H](n3ccc(=O)[nH]c3=O)[C@H](O)[C@@H]2O)CCNCC1]

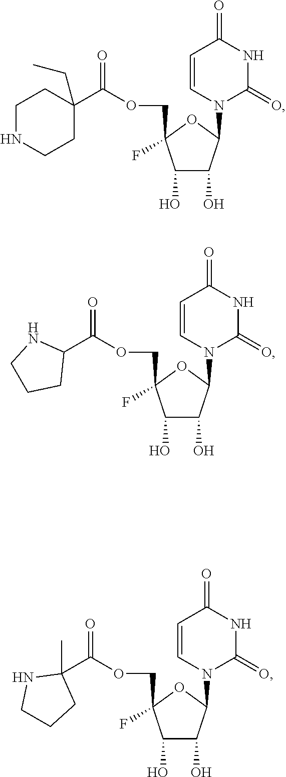 [SMILES: CCC1(C(=O)OC[C@@]2(F)O[C@@H](n3ccc(=O)[nH]c3=O)[C@H](O)[C@@H]2O)CCNCC1 ; O=C(OC[C@@]1(F)O[C@@H](n2ccc(=O)[nH]c2=O)[C@H](O)[C@@H]1O)C1CCCN1 ; CC1(C(=O)OC[C@@]2(F)O[C@@H](n3ccc(=O)[nH]c3=O)[C@H](O)[C@@H]2O)CCCN1]

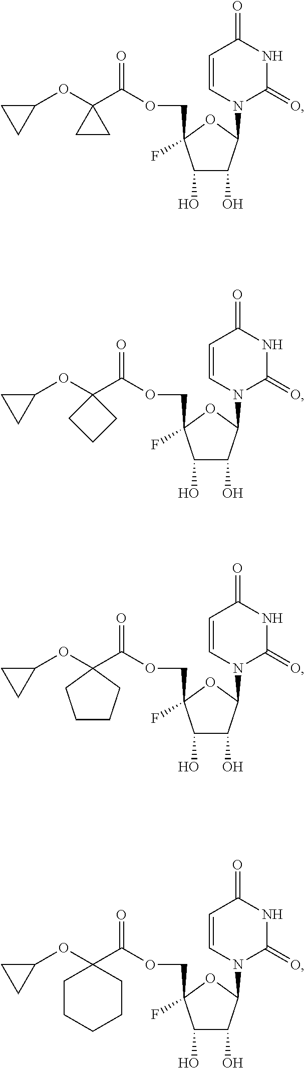 [SMILES: O=C(OC[C@@]1(F)O[C@@H](n2ccc(=O)[nH]c2=O)[C@H](O)[C@@H]1O)C1(OC2CC2)CC1 ; O=C(OC[C@@]1(F)O[C@@H](n2ccc(=O)[nH]c2=O)[C@H](O)[C@@H]1O)C1(OC2CC2)CCC1 ; O=C(OC[C@@]1(F)O[C@@H](n2ccc(=O)[nH]c2=O)[C@H](O)[C@@H]1O)C1(OC2CC2)CCCC1 ; O=C(OC[C@@]1(F)O[C@@H](n2ccc(=O)[nH]c2=O)[C@H](O)[C@@H]1O)C1(OC2CC2)CCCCC1]

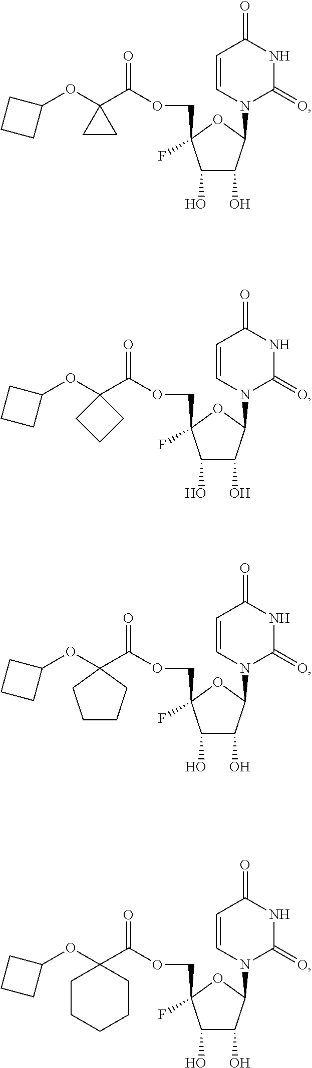 [SMILES: O=C(OC[C@@]1(F)O[C@@H](n2ccc(=O)[nH]c2=O)[C@H](O)[C@@H]1O)C1(OC2CCC2)CC1 ; O=C(OC[C@@]1(F)O[C@@H](n2ccc(=O)[nH]c2=O)[C@H](O)[C@@H]1O)C1(OC2CCC2)CCC1 ; O=C(OC[C@@]1(F)O[C@@H](n2ccc(=O)[nH]c2=O)[C@H](O)[C@@H]1O)C1(OC2CCC2)CCCC1 ; O=C(OC[C@@]1(F)O[C@@H](n2ccc(=O)[nH]c2=O)[C@H](O)[C@@H]1O)C1(OC2CCC2)CCCCC1]

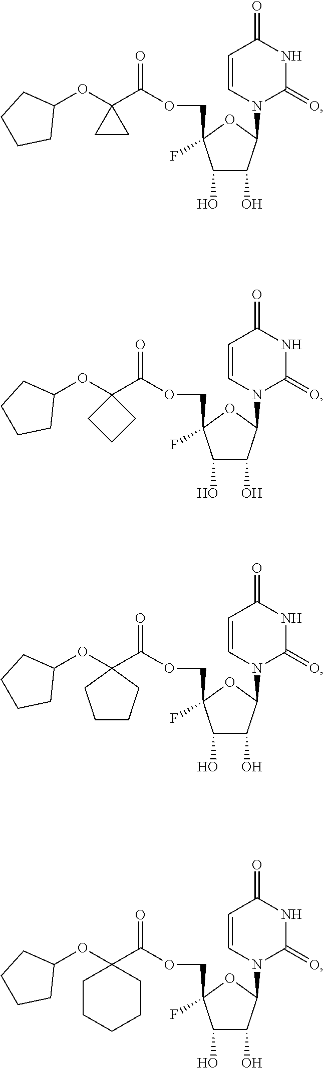 [SMILES: O=C(OC[C@@]1(F)O[C@@H](n2ccc(=O)[nH]c2=O)[C@H](O)[C@@H]1O)C1(OC2CCCC2)CC1 ; O=C(OC[C@@]1(F)O[C@@H](n2ccc(=O)[nH]c2=O)[C@H](O)[C@@H]1O)C1(OC2CCCC2)CCC1 ; O=C(OC[C@@]1(F)O[C@@H](n2ccc(=O)[nH]c2=O)[C@H](O)[C@@H]1O)C1(OC2CCCC2)CCCC1 ; O=C(OC[C@@]1(F)O[C@@H](n2ccc(=O)[nH]c2=O)[C@H](O)[C@@H]1O)C1(OC2CCCC2)CCCCC1]

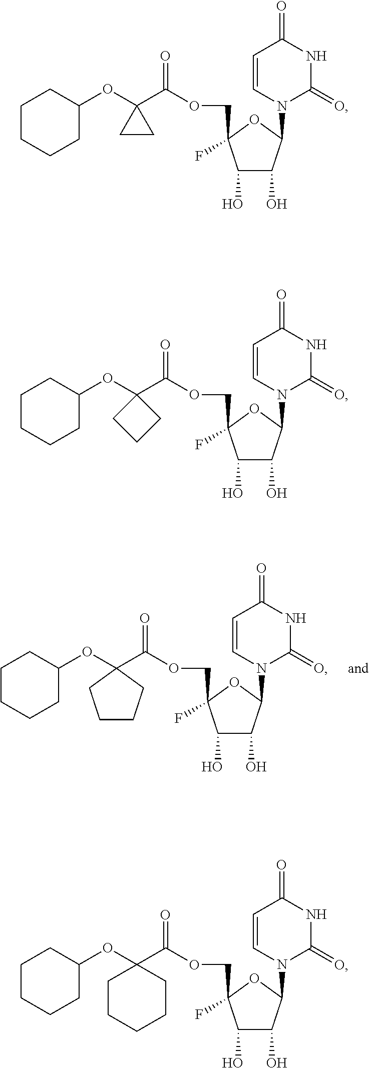 [SMILES: O=C(OC[C@@]1(F)O[C@@H](n2ccc(=O)[nH]c2=O)[C@H](O)[C@@H]1O)C1(OC2CCCCC2)CC1 ; O=C(OC[C@@]1(F)O[C@@H](n2ccc(=O)[nH]c2=O)[C@H](O)[C@@H]1O)C1(OC2CCCCC2)CCC1 ; O=C(OC[C@@]1(F)O[C@@H](n2ccc(=O)[nH]c2=O)[C@H](O)[C@@H]1O)C1(OC2CCCCC2)CCCC1 ; O=C(OC[C@@]1(F)O[C@@H](n2ccc(=O)[nH]c2=O)[C@H](O)[C@@H]1O)C1(OC2CCCCC2)CCCCC1]

or a pharmaceutically acceptable salt, solvate, or polymorph thereof

Methods for treating a virus infection disclosed herein, e.g., enterovirus, RSV, influenza, VEEV, EEEV, HCV and other viruses provided herein, in a subject are also provided. The methods comprise administering the compounds of this disclosure to provide at least two direct acting antiviral agents (DAAs), for example molnupiravir or ribavirin, for a duration of no more than twelve weeks, or for another duration as set forth herein, for example for acute infections for less than one week, e.g., 5 days. Preferably, the two or more direct acting antiviral agents (DAAs) are administered in amounts effective to provide a sustained virological response (SVR) or achieve another desired measure of effectiveness in a subject. In some embodiments, the methods further comprise administering an inhibitor of cytochrome P-450 (such as ritonavir) to the subject to improve the pharmacokinetics or bioavailability of one or more of the DAAs.

As another aspect, methods for treating a virus infection disclosed herein, e.g., enterovirus, RSV, influenza, VEEV, EEEV, HCV and other viruses provided herein, in a subject are provided comprising administering (a) protease inhibitor, (b) at least one polymerase inhibitor, wherein at least one is a polymerase of this disclosure and combinations thereof, with or without (c) molnupiravir. Preferably, the compounds are administered in amounts effective to provide high rates of SVR or another measure of effectiveness in the subject. As non-limiting examples, the compounds can be co-formulated and administered once daily, and the treatment regimen preferably lasts for one to five days to a week.

As still another aspect, methods for treating a virus infection disclosed herein, e.g., enterovirus, RSV, influenza, VEEV, EEEV, HCV and other viruses provided herein, in a subject are provided comprising administering at least two DAAs, wherein one of the DAAs is a compound of this disclosure. Preferably, the at least two DAAs are administered to the subjects in amounts effective to result in SVR or another measure of effectiveness in at least about 70% of the population, preferably at least 90% of the population.

In the foregoing methods as well as methods described herein below, the DAAs can be selected from the group consisting of protease inhibitors, nucleoside or nucleotide polymerase inhibitors (one of which is provided herein), non-nucleoside polymerase inhibitors, NS3B inhibitors, NS4A inhibitors, NS5A inhibitors, NS5B inhibitors, cyclophilin inhibitors, and combinations of any of the foregoing. For example, in some embodiments, the DAAs used in the present methods comprise or consist of at least one HCV protease inhibitor and at least one HCV polymerase inhibitor provided herein.

In some embodiments, the at least two DAAs comprise at least one viral inhibitor, e.g., an inhibitor of enterovirus, RSV, influenza, VEEV, EEEV, HCV and other viruses provided herein, and at least one NS5A inhibitor. By way of example, the polymerase inhibitor of this disclosure can be administered at a total daily dosage from about 100 mg to about 250 mg, and the NS5A inhibitor can be administered in a total daily dose from about 25 mg to about 200 mg. Ritonavir (or another cytochrome P-450 3A4 inhibitor) can be co-administered with to improve the pharmacokinetics and bioavailability of the compounds.

In the foregoing methods as well as methods described herein, the DAAs can be administered in any effective dosing schemes and/or frequencies, for example, they can each be administered daily. Each DAA can be administered either separately or in combination, and each DAA can be administered at less once a day, at least twice a day, or at least three times a day.

In some aspects, the present technology provides methods for treating a virus infection disclosed herein, e.g., enterovirus, RSV, influenza, VEEV, EEEV, HCV and other viruses provided herein, in a subject are provided comprising administering to a subject in need thereof at least two DAAs, wherein the subject is not administered with interferon during said duration. In some aspects, the at least two DAAs with or without ribavirin are administered in an amount effective to result in SVR. Some methods further comprise administering an inhibitor of cytochrome P450 to the subject.

The duration of the treatment regimens in some aspects is no more than sixteen weeks (e.g., the duration being 16 weeks; or the duration being 14, 12 or 10, 9, 8, 7, 6, 5, 4, 3, 2, or 1 weeks or daily for 1-7 days, e.g. one, two, three, four, five, six, or seven days). The treatment includes administering ribavirin but does not include administering interferon. The treatment may include administering ritonavir or another CYP3A4 inhibitor (e.g., cobicistat) if one of the DAAs requires pharmacokinetic enhancement. The two DAAs can be administered concurrently or sequentially. For example, one DAA can be administered once daily, and the other DAA can be administered twice daily. For another example, the two DAAs are administered once daily. For yet another example, the two DAAs are co-formulated in a single composition and administered concurrently (e.g., once daily).

As a further aspect, methods for treating enterovirus in a subject are provided. The methods comprise administering nucleoside or nucleotide compounds of this disclosure. In addition, the methods comprise administering nucleoside or nucleotide compounds of this disclosure in combination with a second antiviral agent active against enteroviruses. In certain embodiments, the disclosure relates to methods of treating a subject diagnosed with an infection caused by enterovirus or preventing an enterovirus infection by administration of a compound or composition disclosed herein. In certain embodiments, the subject is immune-compromised, immune-deficient or immune-suppressed (i.e., a subject in whom any part of the immune system is not working normally, or is working sub-normally, in other words in whom any part of the immune response, or an immune activity is reduced or impaired, whether due to disease or clinical intervention or other treatment, or in any way). In certain embodiments, the nucleoside or nucleotide compounds of this disclosure can be combined with a second antiviral agent as provided in Anasir et al., J Biomed Sci (2021) 28, 10:5-12. Anasir et al. provide a review of antiviral agents for treating enteroviruses, the disclosure of which is incorporated herein by reference in its entirety.

The combination therapy may provide “synergy” and “synergistic effect”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together.

Also disclosed are methods of producing a drug triphosphate, the method comprising: providing a plurality of cells; contacting the plurality of cells with a disclosed compound, e.g., a compound Formula XXIXc; and incubating the plurality of cells and the amount of the compound or pharmaceutical composition for period effective to form the drug triphosphate. In some instances, the plurality of cells are in vivo. In is contemplated that in some instances, the contacting is administering to a subject in need thereof.

### Cancer

In a typical embodiment, the disclosure relates to a method treating cancer comprising administering to a patient a compound disclosed herein. In some embodiments, the disclosure relates to a compound disclosed herein, or a pharmaceutically acceptable salt thereof for uses in treating cancer.

In some embodiments, the disclosure relates to a compound disclosed herein, or a pharmaceutically acceptable salt thereof, as defined herein for use in the treatment of cancer of the breast, colorectum, lung (including small cell lung cancer, non-small cell lung cancer and bronchioalveolar cancer) and prostate.

In some embodiments, the disclosure relates to a compound disclosed herein, or a pharmaceutically acceptable salt thereof, as defined herein for use in the treatment of cancer of the bile duct, bone, bladder, head and neck, kidney, liver, gastrointestinal tissue, oesophagus, ovary, endometrium, pancreas, skin, testes, thyroid, uterus, cervix and vulva, and of leukaemias (including ALL and CML), multiple myeloma and lymphomas.

In some embodiments, the disclosure relates to a compound disclosed herein, or a pharmaceutically acceptable salt thereof, as defined herein for use in the treatment of lung cancer, prostate cancer, melanoma, ovarian cancer, breast cancer, endometrial cancer, kidney cancer, gastric cancer, sarcomas, head and neck cancers, tumors of the central nervous system and their metastases, and also for the treatment of glioblastomas.

In some embodiments, compounds disclosed herein could be used in the clinic either as a single agent by itself or in combination with other clinically relevant agents. This compound could also prevent the potential cancer resistance mechanisms that may arise due to mutations in a set of genes.

The anti-cancer treatment defined herein may be applied as a sole therapy or may involve, in addition to the compound of the disclosure, conventional surgery or radiotherapy or chemotherapy. Such chemotherapy may include one or more of the following categories of antitumour agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon;
(ii) cytostatic agents such as anti-estrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents that inhibit cancer cell invasion (for example metalloproteinase inhibitors like marimastat and inhibitors of urokinase plasminogen activator receptor function); (iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab), farnesyl transferase inhibitors, tyrosine kinase inhibitors and serine/threonine kinase inhibitors, for example inhibitors of the epidermal growth factor family (for example EGFR family tyrosine kinase inhibitors such as: N-(3-chloro-4-fluorophenyl)-7-methoxy-6-(3-morpholinopropoxy)quinazolin-4-amine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;
(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ° CvP3 function and angiostatin);
(vi) vascular damaging agents such as Combretastatin A4;
(vii) antisense therapies, for example those which are directed to the targets listed above, such as an anti-ras antisense;
(viii) gene therapy approaches, including for example approaches to replace aberrant genes such as aberrant p53 or aberrant BRCA1 or BRCA2, GDEPT (gene-directed enzyme prodrug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

Such conjoint treatment may be achieved by way of the simultaneous, sequential or separate dosing of the individual components of the treatment. Such combination products employ the compounds of this disclosure, or pharmaceutically acceptable salts thereof, within the dosage range described hereinbefore and the other pharmaceutically-active agent within its approved dosage range.

In one aspect of the disclosure, an “infection” or “bacterial infection” refers to an infection caused by Acinetobacter spp, Bacteroides spp, Burkholderia spp, Campylobacter spp, Chlamydia spp, Chlamydophila spp, Clostridium spp, Enterobacter spp, Enterococcus spp, Escherichia spp, Fusobacterium spp, Gardnerella spp, Haemophilus spp, Helicobacter spp, Klebsiella spp, Legionella spp, Moraxella spp, Morganella spp, Mycoplasma spp, Neisseria spp, peptococcus spp Peptostreptococcus spp, Proteus spp, Pseudomonas spp, Salmonella spp, Serratia spp., Staphylococcus spp, Streptococcus spp, Stenotrophomonas spp, or Ureaplasma spp.

In one aspect of the disclosure, an “infection” or “bacterial infection” refers to an infection caused by Acinetobacter baumanii, Acinetobacter haemolyticus, Acinetobacter junii, Acinetobacter johnsonii, Acinetobacter lwoffi, Bacteroides bivius, Bacteroides fragilis, Burkholderia cepacia, Campylobacter jejuni, Chlamydia pneumoniae, Chlamydia urealyticus, Chlamydophila pneumoniae, Clostridium difficile, Enterobacter aerogenes, Enterobacter cloacae, Enterococcus faecalis, Enterococcus faecium, Escherichia coli, Gardnerella vaginalis, Haemophilus par influenzae, Haemophilus influenzae, Helicobacter pylori, Klebsiella pneumoniae, Legionella pneumophila, methicillin-resistant Staphylococcus aureus, methicillin-susceptible Staphylococcus aureus, Moraxella catarrhalis, Morganella morganii, Mycoplasma pneumoniae, Neisseria gonorrhoeae, penicillin-resistantpenicillin-resistant Streptococcus pneumoniae, penicillin-susceptible Streptococcus pneumoniae, Peptostreptococcus magnus, Peptostreptococcus micros, Peptostreptococcus anaerobius, Peptostreptococcus asaccharolyticus, Peptostreptococcus prevotii, Peptostreptococcus tetradius, Peptostreptococcus vaginalis, Proteus mirabilis, Pseudomonas aeruginosa, quino lone-resistant Staphylococcus aureus, quinolone-resistant Staphylococcus epidermis, Salmonella typhi, Salmonella paratyphi, Salmonella enteritidis, Salmonella typhimurium, Serratia marcescens, Staphylococcus aureus, Staphylococcus epidermidis, Staphylococcus saprophyticus, Streptococcus agalactiae, Streptococcus pneumoniae, Streptococcus pyogenes, Stenotrophomonas maltophilia, Ureaplasma urealyticum, vancomycin-resistant Enterococcus faecium, vancomycin-resistant Enterococcus faecalis, vancomycin-resistant Staphylococcus aureus, vancomycin-resistant Staphylococcus epidermis, Mycobacterium tuberculosis, Clostridium perfringens, Klebsiella oxytoca, Neisseria meningitidis, proteus vulgaris, or coagulase-negative Staphylococcus (including Staphylococcus lugdunensis, Staphylococcus capitis, Staphylococcus hominis, or Staphylococcus saprophytic).

In one aspect of the disclosure “infection” or “bacterial infection” refers to aerobes, obligate anaerobes, facultative anaerobes, gram-positive bacteria, gram-negative bacteria, gram-variable bacteria, or atypical respiratory pathogens.

In some embodiments, the disclosure relates to treating a bacterial infection such as a gynecological infection, a respiratory tract infection (RTI), a sexually transmitted disease, or a urinary tract infection.

In some embodiments, the disclosure relates to treating a bacterial infection such as an infection caused by drug resistant bacteria.

In some embodiments, the disclosure relates to treating a bacterial infection such as community-acquired pneumoniae, hospital-acquired pneumoniae, skin & skin structure infections, gonococcal cervicitis, gonococcal urethritis, febrile neutropenia, osteomyelitis, endocarditis, urinary tract infections and infections caused by drug resistant bacteria such as penicillin-resistantpenicillin-resistant Streptococcus pneumoniae, methicillin-resistant Staphylococcus aureus, methicillin-resistant Staphylococcus epidermidis and vancomycin-resistant enterococci, syphilis, ventilator-associated pneumonia, intra-abdominal infections, gonorrhoeae, meningitis, tetanus, or tuberculosis.

In some embodiments, the disclosure relates to treating a fungal infections such as infections caused by tinea versicolor, microsporum, trichophyton, epidermophyton, candidiasis, cryptococcosis, or aspergillosis.

In some embodiments, the disclosure relates to treating an infection caused by protozoa including, but not limited to, malaria, amoebiasis, giardiasis, toxoplasmosis, cryptosporidiosis, trichomoniasis, leishmaniasis, sleeping sickness, or dysentery.

Certain compounds disclosed herein are useful to prevent or treat an infection of a malarial parasite in a subject and/or for preventing, treating and/or alleviating complications and/or symptoms associated therewith and can then be used in the preparation of a medicament for the treatment and/or prevention of such disease. The malaria may be caused by Plasmodium falciparum, P. vivax, P. ovale, or P. malariae.

### Formulations

Pharmaceutical compositions disclosed herein may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When a compound of the disclosure contains a hydrogen-donating heteroatom (e.g., NH), the disclosure also covers salts and/or isomers formed by the transfer of the hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier that releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as a prodrug are known, for example, in Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids. It has been shown that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.”

Pharmaceutical compositions for use in the present disclosure typically comprise an effective amount of a compound of Formulas XXIX-XXXIVb and a pharmaceutically acceptable excipient or an effective amount of a disclosed compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g., about 10, 25, 50, 100, 200, 300 or 400 mg per unit dosage.

The compounds can be administered by a variety of routes including the oral, ° Cular, rectal, transdermal, subcutaneous, sublingual, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, every other day, twice weekly, or weekly, more often between 0.1 and 500 mg, such as between 1 and 250 mg, for example about 5, 10, 20, 50, 100, 150, 200 or 250 mg, per kilogram body weight of the patient per day, every other day, twice weekly, or weekly, which may be administered as a single daily, every other day, twice weekly, or weekly dose, or divided over one or more daily, every other day, twice weekly, or weekly doses. The amount(s) to be administered, the route of administration and the further treatment regimen may be determined by the treating clinician, depending on factors such as the age, gender and general condition of the patient and the nature and severity of the disease/symptoms to be treated. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

For an oral administration form, the compound can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, cornstarch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the compounds, if desired with the substances customary therefore such as solubilizers, emulsifiers or further auxiliaries are brought into solution, suspension, or emulsion. The compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds of formula I with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that these compositions can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na-CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release protein delivery systems. Proteins can be entrapped in the poly(lactide-co-glycolide) microsphere depot by a number of methods, including formation of a water-in-oil emulsion with water-borne protein and organic solvent-borne polymer (emulsion method), formation of a solid-in-oil suspension with solid protein dispersed in a solvent-based polymer solution (suspension method), or by dissolving the protein in a solvent-based polymer solution (dissolution method). One can attach poly(ethylene glycol) to proteins (PEGylation) to increase the in vivo half-life of circulating therapeutic proteins and decrease the chance of an immune response.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

It is appreciated that nucleosides of the present invention have several chiral centers and may exist in and be isolated in optically active and racemic forms. Some compounds may exhibit polymorphism. It is to be understood that the present invention encompasses any racemic, optically active, diastereomeric, polymorphic, or stereoisomeric form, or mixtures thereof, of a compound of the invention, which possess the useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase).

Carbons of the nucleoside are chiral, their nonhydrogen substituents (the base and the CHOR groups, respectively) can be either cis (on the same side) or trans (on opposite sides) with respect to the sugar ring system. The four optical isomers therefore are represented by the following configurations (when orienting the sugar moiety in a horizontal plane such that the oxygen atom is in the back): cis (with both groups “up”, which corresponds to the configuration of naturally ° Ccurring 3-D nucleosides), cis (with both groups “down”, which is a nonnaturally ° Ccurring β-L configuration), trans (with the C2′ substituent “up” and the C4′ substituent “down”), and trans (with the C2′ substituent “down” and the C4′ substituent “up”). The “D-nucleosides” are cis nucleosides in a natural configuration and the “L-nucleosides” are cis nucleosides in the nonnaturally ° Ccurring configuration.

Likewise, most amino acids are chiral (designated as L or D, wherein the L enantiomer is the naturally ° Ccurring configuration) and can exist as separate enantiomers.

Examples of methods to obtain optically active materials are known in the art, and include at least the following. i) physical separation of crystals—a technique whereby macroscopic crystals of the individual enantiomers are manually separated. This technique can be used if crystals of the separate enantiomers exist, i.e., the material is a conglomerate, and the crystals are visually distinct; ii) simultaneous crystallization—a technique whereby the individual enantiomers are separately crystallized from a solution of the racemate, possible only if the latter is a conglomerate in the solid state; iii) enzymatic resolutions—a technique whereby partial or complete separation of a racemate by virtue of differing rates of reaction for the enantiomers with an enzyme; iv) enzymatic asymmetric synthesis—a synthetic technique whereby at least one step of the synthesis uses an enzymatic reaction to obtain an enantiomerically pure or enriched synthetic precursor of the desired enantiomer; v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries; vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer; vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer; viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions; ix) enantiospecific synthesis from non-racemic precursors—a synthetic technique whereby the desired enantiomer is obtained from non-chiral starting materials and where the stereochemical integrity is not or is only minimally compromised over the course of the synthesis; x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions; xi) chiral gas chromatography—a technique whereby the racemate is volatilized and enantiomers are separated by virtue of their differing interactions in the gaseous mobile phase with a column containing a fixed non-racemic chiral adsorbent phase; xii) extraction with chiral solvents—a technique whereby the enantiomers are separated by virtue of preferential dissolution of one enantiomer into a particular chiral solvent; xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation ° Ccurs as a result of the non-racemic chiral nature of the membrane that allows only one enantiomer of the racemate to pass through. Chiral chromatography, including simulated moving bed chromatography, is used in one embodiment. A wide variety of chiral stationary phases are commercially available.

Some of the compounds described herein contain olefinic double bonds and unless otherwise specified, are meant to include both E and Z geometric isomers.

In addition, some of the nucleosides described herein, may exist as tautomers, such as, keto-enol tautomers. The individual tautomers as well as mixtures thereof are intended to be encompassed within the compounds of the present invention.

### Combination Therapies

The compound described herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, anti-narcoleptics, and antiviral agents. In a particular embodiment, the antiviral agent is a non-CNS targeting antiviral compound. “Adjunctive administration”, as used herein, means the compound can be administered in the same dosage form or in separate dosage forms with one or more other active agents. The additional active agent(s) can be formulated for immediate release, controlled release, or combinations thereof.

Specific examples of compounds that can be adjunctively administered with the compounds include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, IPrindole, IPsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

In certain embodiments, the exemplary compounds and pharmaceutical compositions can be administered in combination with another antiviral agent(s) such as abacavir, acyclovir, acyclovir, adefovir, amantadine, amprenavir, ampligen, arbidol, atazanavir, atripla, balapiravir, BCX4430, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, or zidovudine and combinations thereof.

In exemplified embodiments, the exemplary compounds and pharmaceutical compositions can be administered in combination with

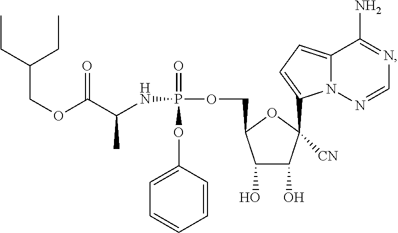 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

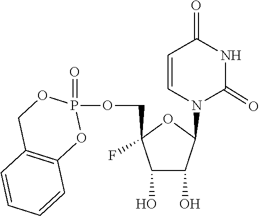 [SMILES: O=c1ccn([C@@H]2O[C@](F)(COP3(=O)OCc4ccccc4O3)[C@@H](O)[C@H]2O)c(=O)[nH]1]

can be administered in combination with

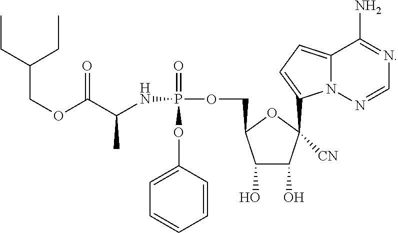 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

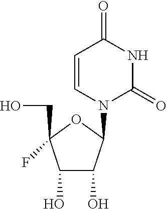 [SMILES: O=c1ccn([C@@H]2O[C@](F)(CO)[C@@H](O)[C@H]2O)c(=O)[nH]1]

can be administered in combination with

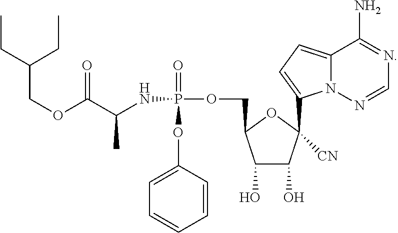 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

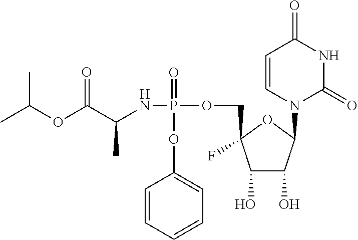 [SMILES: CC(C)OC(=O)[C@H](C)NP(=O)(OC[C@@]1(F)O[C@@H](n2ccc(=O)[nH]c2=O)[C@H](O)[C@@H]1O)Oc1ccccc1]

can be administered in combination with

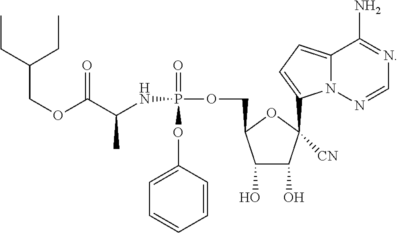 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments,

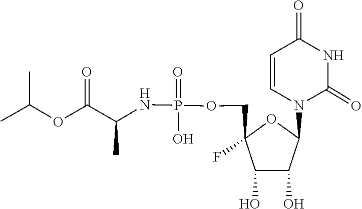 [SMILES: CC(C)OC(=O)[C@H](C)NP(=O)(O)OC[C@@]1(F)O[C@@H](n2ccc(=O)[nH]c2=O)[C@H](O)[C@@H]1O]

can be administered in combination with

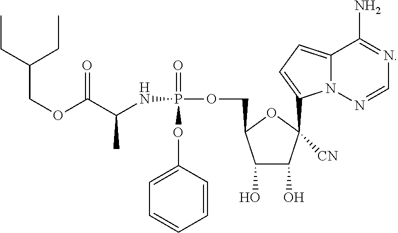 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In exemplified embodiments, the pharmaceutical composition comprises a compound of any one of Formulas XXIX-XXXIVb and a pharmaceutically acceptable excipient.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is formulation is for oral delivery.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a capsule, a tablet, a cachet, a pill, a powder, a granule, an elixir, a tincture, a suspension, a syrup, or an emulsion.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a formulation is for oral delivery and is a solid dosage form.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a formulation for parenteral delivery.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a formulation for parenteral delivery such as bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a formulation for parenteral delivery such as subcutaneous, intravenous, intramuscular, intra-articular, intra-synovial, intrasternal, intrathecal, intrahepatic, intralesional, and intracranial injections or infusion techniques.

In exemplified embodiments, the pharmaceutical composition comprises a compound of any one of Formulas XXIX-XXXIVb, or any compound species or combination of compound species as disclosed herein, or a pharmaceutically acceptable salt, solvate, or polymorph thereof, and a pharmaceutically acceptable excipient that is a formulation for pulmonary delivery.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a formulation for pulmonary delivery comprising a propellant.

In exemplified embodiments, the pharmaceutical composition comprises a compound of Formulas XXIX-XXXIVb, or any compound species or combination of compound species as disclosed herein, or a pharmaceutically acceptable salt, solvate, or polymorph thereof, and a pharmaceutically acceptable excipient that is a formulation for pulmonary delivery comprising a propellant such as compressed air, ethanol, nitrogen, carbon dioxide, nitrous oxide, hydrofluoroalkanes (HFA), 1,1,1,2,-tetrafluoroethane, 1,1,1,2,3,3,3-heptafluoropropane or combinations thereof.

### ASPECTS

The following listing of exemplary aspects supports and is supported by the disclosure provided herein.

Aspect 1. A method of treating or preventing a viral infection comprising administering to a subject in need thereof an effective amount of a pharmaceutical composition comprising a pharmaceutically acceptable excipient and a compound of Formula XXIXc:

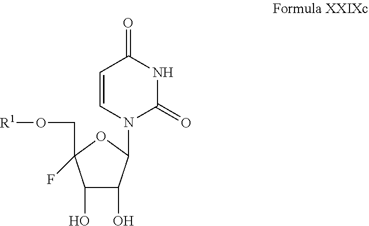

or a pharmaceutical or physiological salt thereof,
wherein R¹ is selected from a group having a structure represented by a formula:

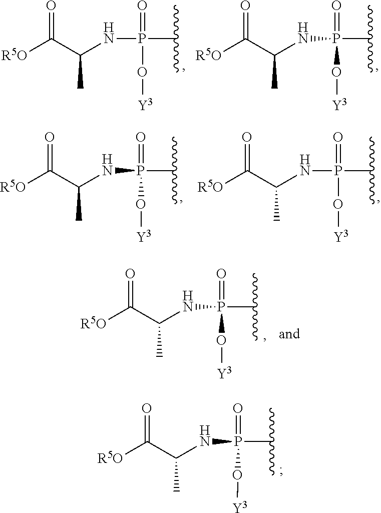

wherein Y³ is hydrogen, aryl, heteroaryl, or heterocyclyl; and wherein Y³ is optionally substituted with one or more, the same or different, R¹⁰;
wherein R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid; and wherein R⁵ is optionally substituted with one or more, the same or different, R¹⁰;
wherein R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl.
   Aspect 2. The method of claim Aspect 1, wherein R¹ is a group having a structure represented by a formula:

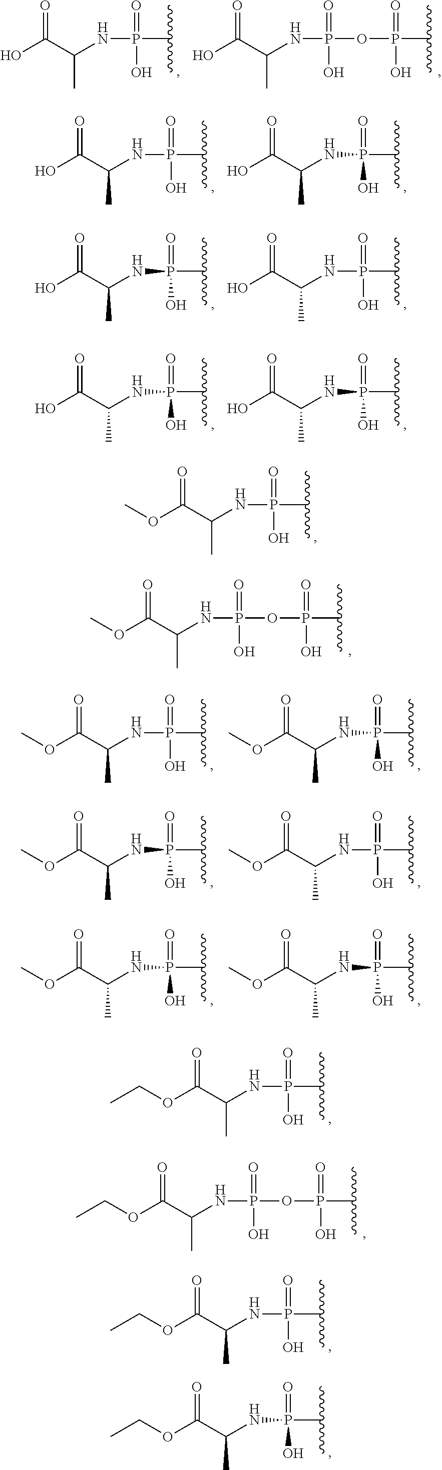

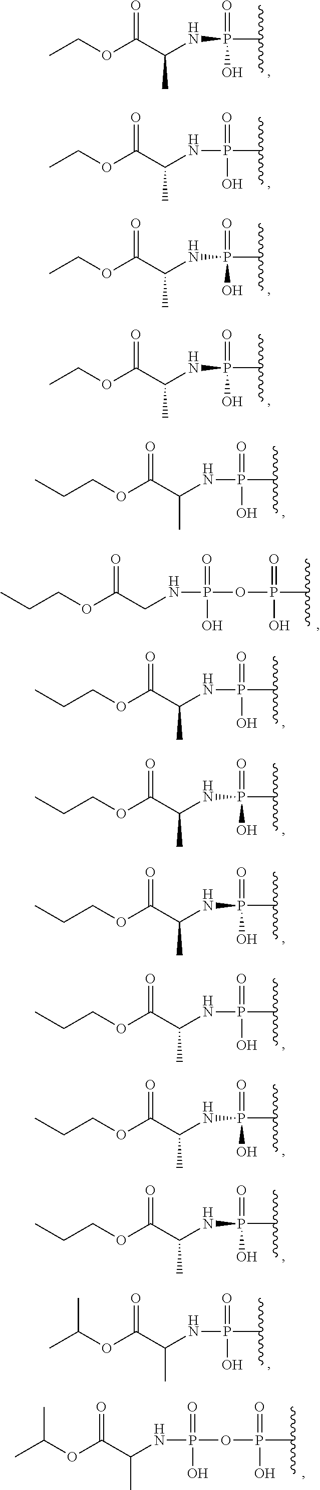

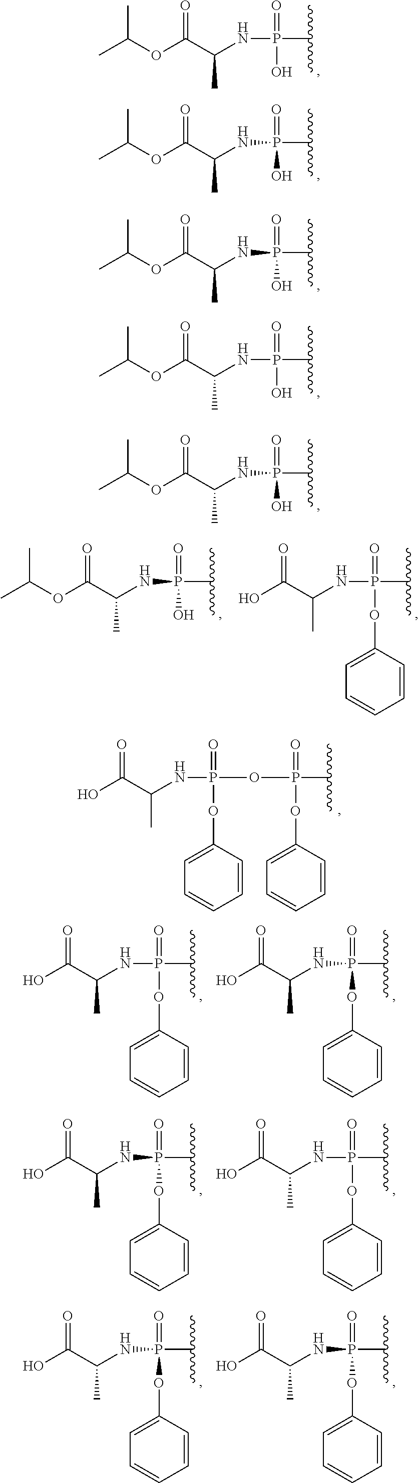

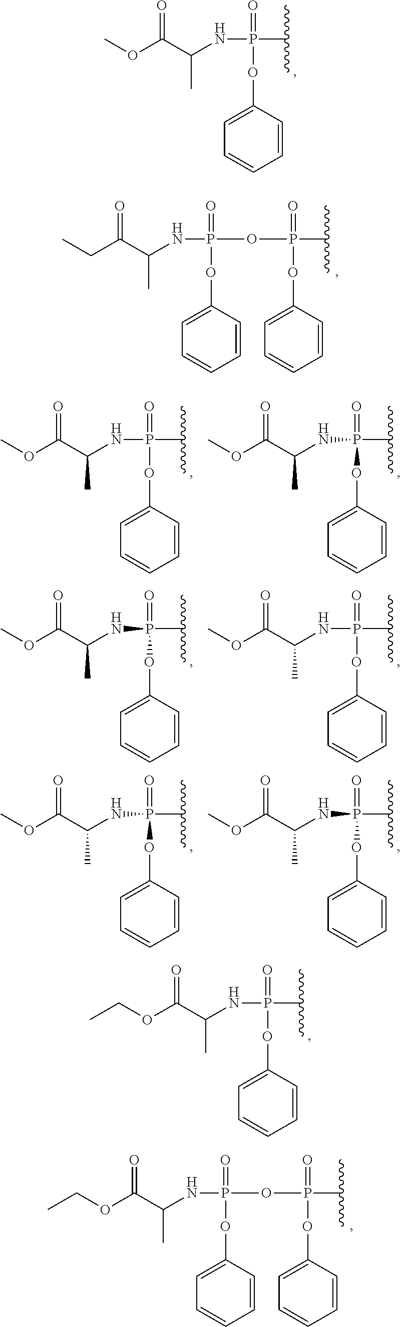

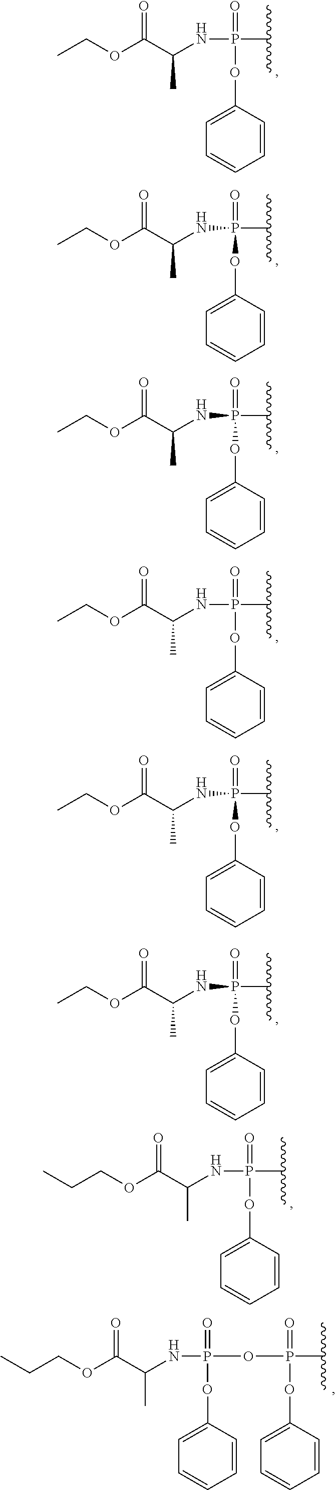

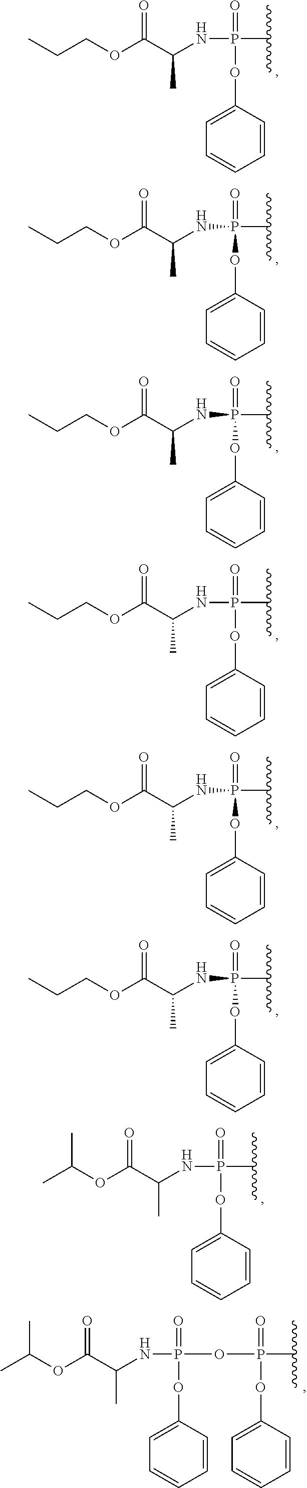

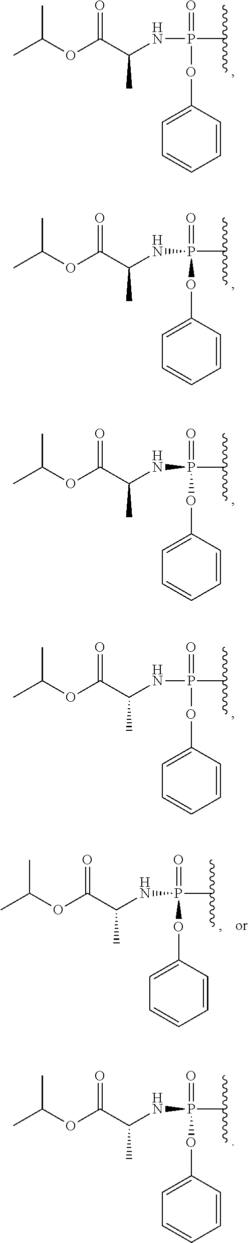

Aspect 3. The method of claim Aspect 2, wherein R¹ is a group having a structure represented by a formula:

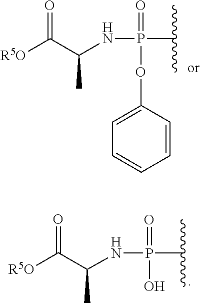

Aspect 4. The method of any one of claims Aspect 1-Aspect 3, wherein R⁵ is selected from lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.
Aspect 5. The method of claim Aspect 4, wherein R⁵ is selected from methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, and cyclohexyl.
Aspect 6. The method of claim Aspect 4, wherein R⁵ is selected from methyl, ethyl, propyl, isopropyl, butyl, s-butyl, t-butyl, pentyl, s-pentyl, t-pentyl, neopentyl, 3-pentyl, hexyl, t-hexyl, and 4-septyl.
Aspect 7. The method of claim Aspect 4, wherein R⁵ is selected from methyl, ethyl, propyl, isopropyl, butyl, s-butyl, t-butyl, pentyl, s-pentyl, t-pentyl, neopentyl, and 3-pentyl.
Aspect 8. The method of any one of claim 1, wherein the compound is selected from a structure represented by a formula:

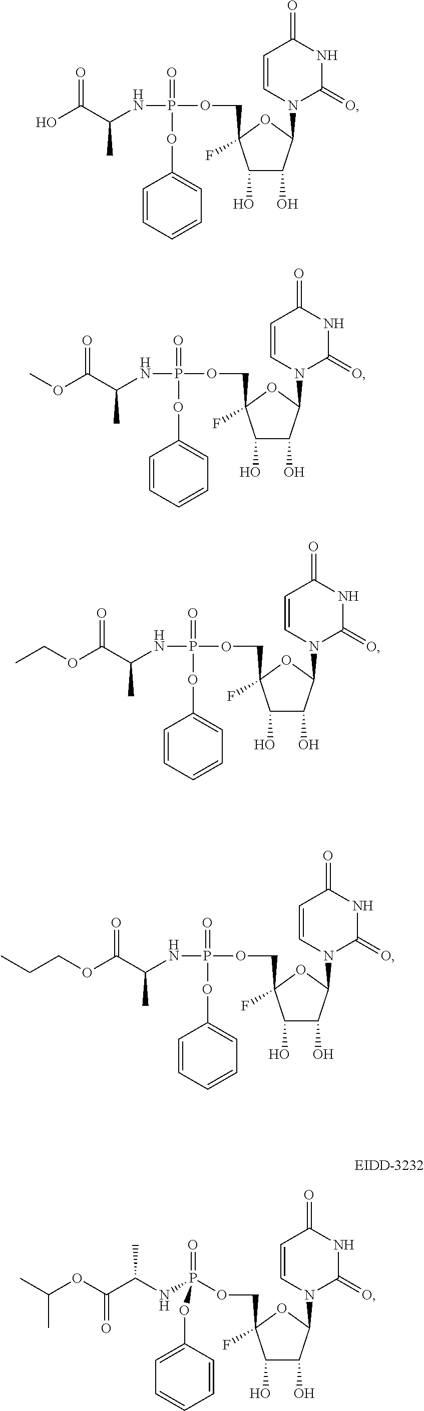 [SMILES: C[C@H](NP(=O)(OC[C@@]1(F)O[C@@H](n2ccc(=O)[nH]c2=O)[C@H](O)[C@@H]1O)Oc1ccccc1)C(=O)O ; COC(=O)[C@H](C)NP(=O)(OC[C@@]1(F)O[C@@H](n2ccc(=O)[nH]c2=O)[C@H](O)[C@@H]1O)Oc1ccccc1 ; CCOC(=O)[C@H](C)NP(=O)(OC[C@@]1(F)O[C@@H](n2ccc(=O)[nH]c2=O)[C@H](O)[C@@H]1O)Oc1ccccc1 ; CCCOC(=O)[C@H](C)NP(=O)(OC[C@@]1(F)O[C@@H](n2ccc(=O)[nH]c2=O)[C@H](O)[C@@H]1O)Oc1ccccc1 ; CC(C)OC(=O)[C@H](C)N[P@](=O)(OC[C@@]1(F)O[C@@H](n2ccc(=O)[nH]c2=O)[C@H](O)[C@@H]1O)Oc1ccccc1]

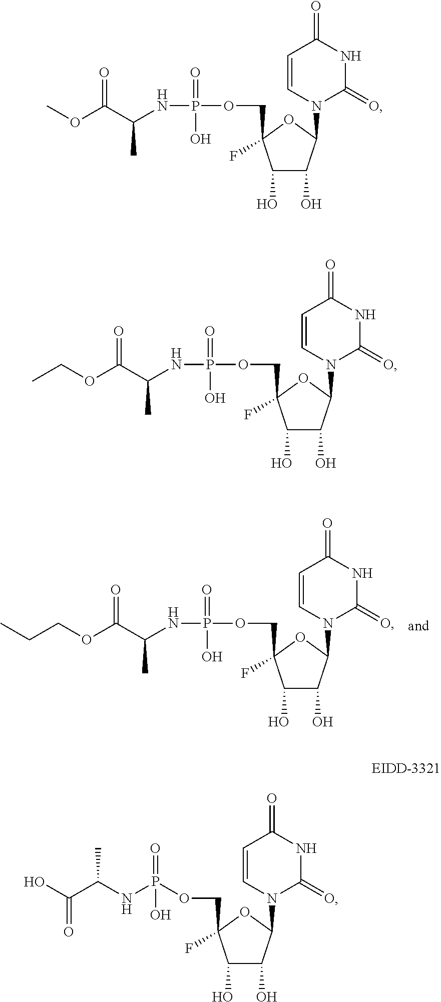 [SMILES: COC(=O)[C@H](C)NP(=O)(O)OC[C@@]1(F)O[C@@H](n2ccc(=O)[nH]c2=O)[C@H](O)[C@@H]1O ; CCOC(=O)[C@H](C)NP(=O)(O)OC[C@@]1(F)O[C@@H](n2ccc(=O)[nH]c2=O)[C@H](O)[C@@H]1O ; CCCOC(=O)[C@H](C)NP(=O)(O)OC[C@@]1(F)O[C@@H](n2ccc(=O)[nH]c2=O)[C@H](O)[C@@H]1O ; C[C@H](NP(=O)(O)OC[C@@]1(F)O[C@@H](n2ccc(=O)[nH]c2=O)[C@H](O)[C@@H]1O)C(=O)O]

and combinations thereof
Aspect 9. The method of claim 1, wherein the compound is a structure represented by a formula:

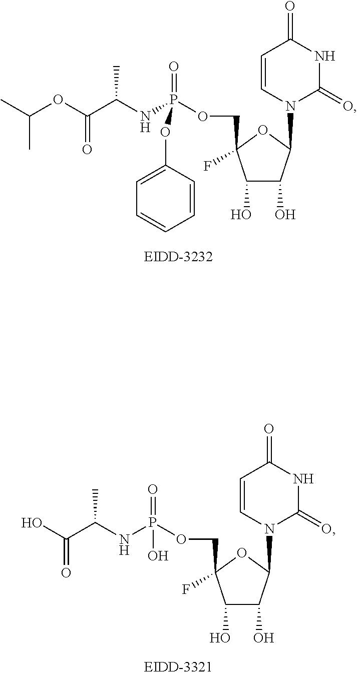 [SMILES: CC(C)OC(=O)[C@H](C)N[P@](=O)(OC[C@@]1(F)O[C@@H](n2ccc(=O)[nH]c2=O)[C@H](O)[C@@H]1O)Oc1ccccc1 ; C[C@H](NP(=O)(O)OC[C@@]1(F)O[C@@H](n2ccc(=O)[nH]c2=O)[C@H](O)[C@@H]1O)C(=O)O]

or a combination thereof.
Aspect 10. The method of any one of claims Aspect 1-Aspect 9, wherein the method further comprises administering a second antiviral agent.
Aspect 11. The method of claim Aspect 10, wherein the second antiviral agent is selected from remdesivir, favipiravir, darunavir, nelfinavir, saquinavir, lopinavir, ritonavir, remdesivir, paxlovid, molnupiravir, ABX464, favilavir, niclosamide, laninamivir, oseltamivir, zanamivir, peramivir, CS-8958, ribavirin, amantadine, rimantadine, tamiphosphor guanidine monoester, or phosphazanamivir or its monoester, disoxaril, pleconaril, pirodavir, vapendavir, pocapavir, azaglutamine, S-nitroso-N-acetyl-penicillamine (SNAP), glyceryl trinitrate (GTN), isosorbide dinitrate (ISDN), glycerrhizin, 5-(3,4-dichlorophenyl) methylhydantoin, AG7088, pleconaril, 3-methylthio-5-aryl-4-isothiazolecarbonitrile, a pyridyl imidazolidinone, ribavirin, mycophenolic acid, 6-azauridine, pyrazofurin, and 3-methylkaempferol, and derivatives, physiological or pharmaceutical salts, or prodrugs thereof, and combinations thereof.
Aspect 12. The method of claim Aspect 11, wherein the second antiviral agent is selected from remdesivir, favipiravir, darunavir, nelfinavir, saquinavir, lopinavir, ritonavir, remdesivir, paxlovid, molnupiravir, ABX464, favilavir, and niclosamide, and derivatives, physiological or pharmaceutical salts, or prodrugs thereof, and combinations thereof.
Aspect 13. The method of claim Aspect 11, wherein the second antiviral agent is selected from laninamivir, oseltamivir, zanamivir, peramivir, CS-8958, ribavirin, amantadine, rimantadine, tamiphosphor guanidine monoester, and phosphazanamivir or its monoester, and derivatives, physiological or pharmaceutical salts, or prodrugs thereof, and combinations thereof.
Aspect 14. The method of any one of claims Aspect 1-Aspect 13, wherein the virus does not infect or substantially infect the liver.
Aspect 15. The method of claim Aspect 14, wherein the viral infection does not comprise a flavivirus infection.
Aspect 16. The method of claim Aspect 15, wherein the flavivirus infection does not comprise a hepatitis virus.
Aspect 17. The method of claim Aspect 16, wherein the hepatitis virus is hepatitis C virus.
Aspect 18. The method of any one of claims Aspect 1-Aspect 16, wherein the viral infection comprises an RNA virus infection.
Aspect 19. The method of any one of claims Aspect 1-Aspect 18, wherein the viral infection comprises a virus selected from coxsackie virus A, B and C, coxsackie A16, EV-D68, EV-A71, rhinovirus, poliovirus, echovirus, picornaviruses, cardioviruses, enteroviruses, erboviruses, hepatovirus, kobuviruses, parechoviruses, teschoviruses, caliciviruses, which include noroviruses, sapoviruses, lagoviruses, vesiviruses, astroviruses, togaviruses, flaviviruses, hepacivirus, coronaviruses, arteriviruses, rhabdoviruses, paramyxoviruses, orthomyxoviruses, hantaviruses, reoviruses, rotaviruses, birnaviruses, chrysoviruses, cystoviruses, hypoviruses partitiviruses, totoviruses, lentiviruses, polyomaviruses, papillomaviruses, adenoviruses, circoviruses, parvoviruses, erythroviruses, betaparvoviruses, amdoviruses, densoviruses, iteraviruses, brevidensoviruses, pefudensoviruses, herpes viruses 1, 2, 3, 4, 5, 6, 7 and 8, poxviruses, hepadnaviruses, pneumovirus, bunyavirus, arenavirus, orthomyxovirus, human coronavirus, SARS coronavirus, MERS coronavirus, Eastern equine encephalitis virus, Western equine encephalitis virus, Venezuelan equine encephalitis virus, Chikungunya virus, Ross River virus, RSV, an influenza virus, Tacaribe virus, Pichinde virus, Junin virus, Lassa fever virus, Lymphocytic Choriomeningitis virus, Rift Valley fever virus, Punta Toro virus, LaCrosse virus, Maporal virus, Heartland virus, and Severe Fever Thrombocytopenia Syndrome virus, and combinations thereof.
Aspect 20. The method of claim Aspect 19, wherein the viral infection comprises an enterovirus.
Aspect 21. The method of claim Aspect 20, wherein the enterovirus is a non-polio enterovirus.
Aspect 22. The method of claim Aspect 20 or claim Aspect 21, wherein the enterovirus or coxsackievirus is selected from bovine enterovirus, human enterovirus A, human enterovirus B, human enterovirus C, human enterovirus D, porcine enterovirus B, simian enterovirus A, human rhinovirus A, human rhinovirus B, rhinovirus C, coxsackie A16, EV-D68, EV-A71, rhinovirus, poliovirus, and echovirus, and combinations thereof.
Aspect 23. The method of claim Aspect 20, wherein the enterovirus comprises an enterovirus selected from the group consisting of coxsackie virus A, B and C, coxsackie A16, EV-D68, EV-A71, rhinovirus, poliovirus, and echovirus.
Aspect 24. The method of claim Aspect 23, wherein the enterovirus comprises a coxsackie virus.
Aspect 25. The method of claim Aspect 23, wherein the enterovirus comprises Coxsackie A16.
Aspect 26. The method of claim Aspect 19, wherein the viral infection is a Togaviridae.
Aspect 27. The method of claim Aspect 26, wherein the virus is selected from Eastern equine encephalitis virus, Western equine encephalitis virus, Venezuelan equine encephalitis virus, Chikungunya virus, and Ross River virus.
Aspect 28. The method of claim Aspect 19, wherein the viral infection is a human coronavirus, SARS coronavirus, and MERS coronavirus infection.
Aspect 29. The method of claim Aspect 28, wherein the viral infection is a SARS-CoV2-infection.
Aspect 30. The method of claim Aspect 29, wherein variants of SARS-CoV-2 but not limited to the more virulent strain originating in Brasil, known as P.1; the variant originating in the United Kingdom, known as 2011501Y.V1, VOC 202012/01, or B.1.1.7; and the variant originating in South Africa, known as 20H/501Y.V2 or B.1.351; as well as further varients and lineages that derive therefrom.
Aspect 31. The method of claim Aspect 19, wherein the viral infection is a orthomyxovirus.
Aspect 32. The method of claim 33, wherein the orthomyxovirus is selected from alphainfluenzavirus, betainfluenzavirus, gammainfluenzavirus, deltainfluenzavirus, isavirus, thogotovirus, and quaranjavirus.
Aspect 33. The method of claim Aspect 31, wherein the viral infection is influenza A virus and influenza B virus.
Aspect 34. The method of claim Aspect 19, wherein the viral infection is a Pneumoviridae.
Aspect 35. The method of claim Aspect 34, wherein the viral infection is RSV.
Aspect 36. The method of claim Aspect 19, wherein the viral infection is an Arenaviridae.
Aspect 37. The method of claim Aspect 36, wherein the viral infection is Tacaribe virus, Pichinde virus, Junin virus, Lassa fever virus, and Lymphocytic Choriomeningitis virus.
Aspect 38. The method of claim Aspect 19, wherein the viral infection is Bunyaviridae.
Aspect 39. The method of claim 38, wherein the viral infection is Rift Valley fever virus, Punta Toro virus, LaCrosse virus, Maporal virus, Heartland virus, and Severe Fever Thrombocytopenia Syndrome virus.
Aspect 40. The method of claim Aspect 19, wherein the viral infection is Flaviviridae.
Aspect 41. The method of claim Aspect 40, wherein the viral infection is Zika virus, Dengue virus 1, Dengue virus 2, Dengue virus 3, Dengue virus 4, West Nile virus, Yellow fever virus, Japanese encephalitis virus, Powassen virus, Usutu virus, and tick-borne encephalitis virus.
Aspect 42. The method of claim Aspect 19, wherein the viral infection is Picornaviridae.
Aspect 43. The method of claim Aspect 42, wherein the viral infection is poliovirus, Coxsackie virus, enterovirus.
Aspect 44. A method of treating or preventing a viral infection comprising administering to a subject in need thereof an effective amount of a pharmaceutical composition a pharmaceutically acceptable excipient and a compound of Formula XXIXc;
wherein the subject is administered a loading dose of the pharmaceutical composition in a first treatment period; and
wherein the subject is administered a treatment dose of the pharmaceutical composition in a second treatment period following the first treatment period;
wherein the compound of Formula XXIXc is a compound having a structure given by the formula:

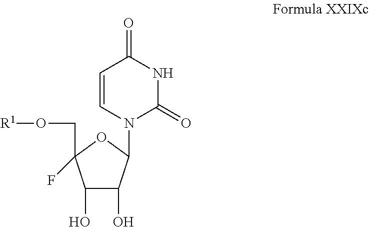

or a pharmaceutical or physiological salt thereof,
wherein R¹ is selected from a group having a structure represented by a formula:

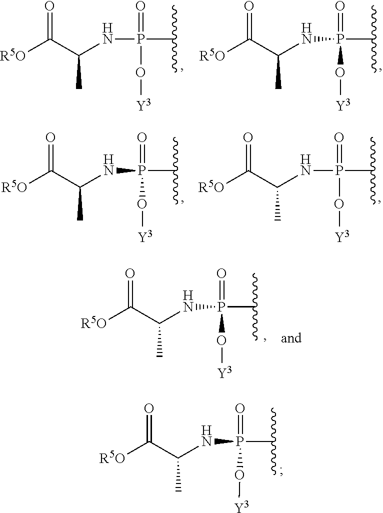

wherein Y³ is hydrogen, aryl, heteroaryl, or heterocyclyl; and wherein Y³ is optionally substituted with one or more, the same or different, R¹⁰;
wherein R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid; and wherein R⁵ is optionally substituted with one or more, the same or different, R¹⁰;
wherein R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl.
   Aspect 45. The method of claim Aspect 44, wherein R¹ is a group having a structure represented by a formula:

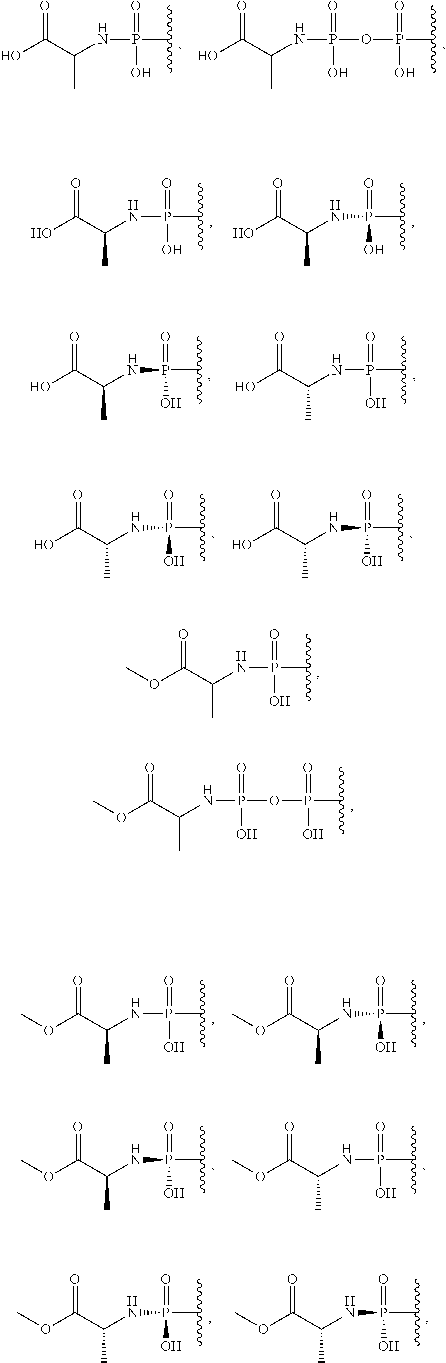

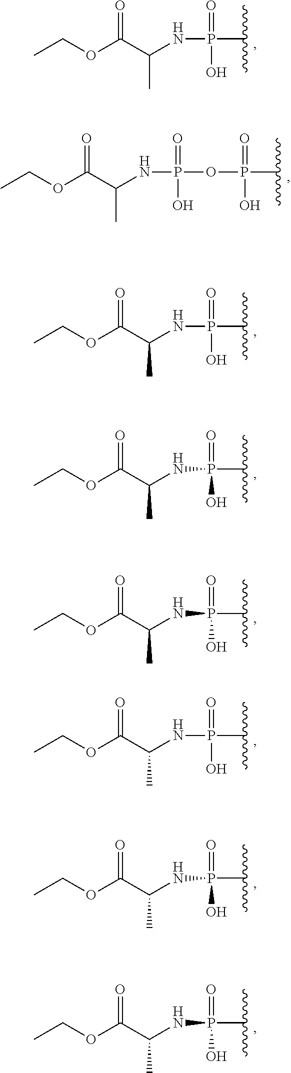

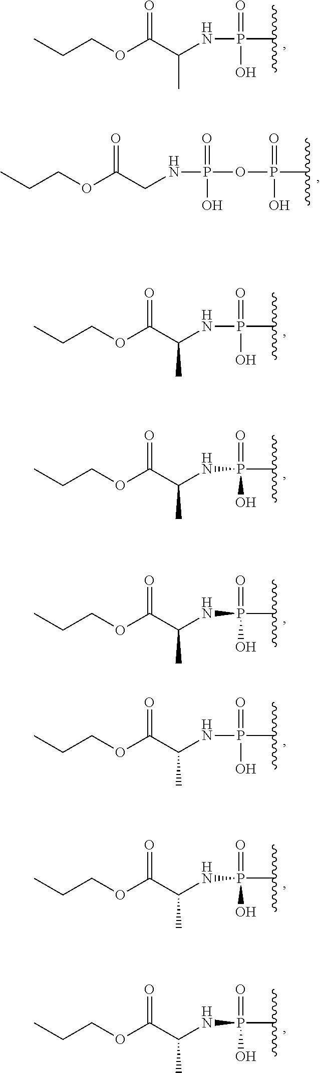

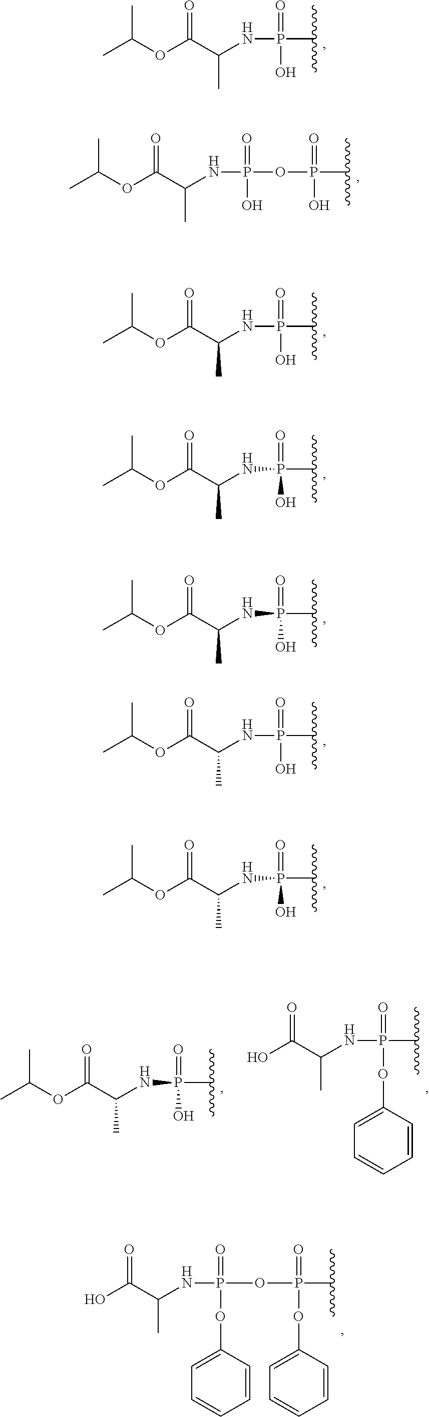

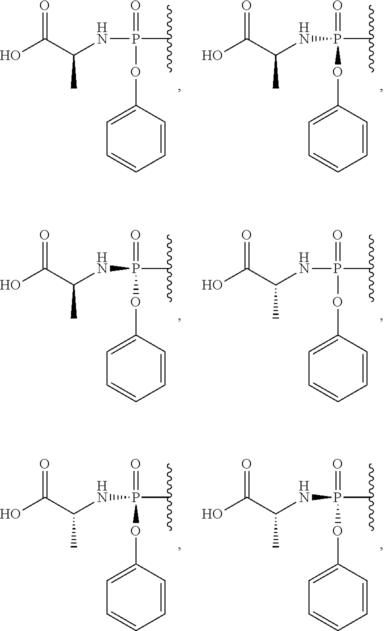 [SMILES: *P(=O)(N[C@@H](C)C(=O)O)Oc1ccccc1 ; *[P@@](=O)(N[C@@H](C)C(=O)O)Oc1ccccc1 ; *[P@](=O)(N[C@@H](C)C(=O)O)Oc1ccccc1 ; *P(=O)(N[C@H](C)C(=O)O)Oc1ccccc1 ; *[P@@](=O)(N[C@H](C)C(=O)O)Oc1ccccc1 ; *[P@](=O)(N[C@H](C)C(=O)O)Oc1ccccc1]

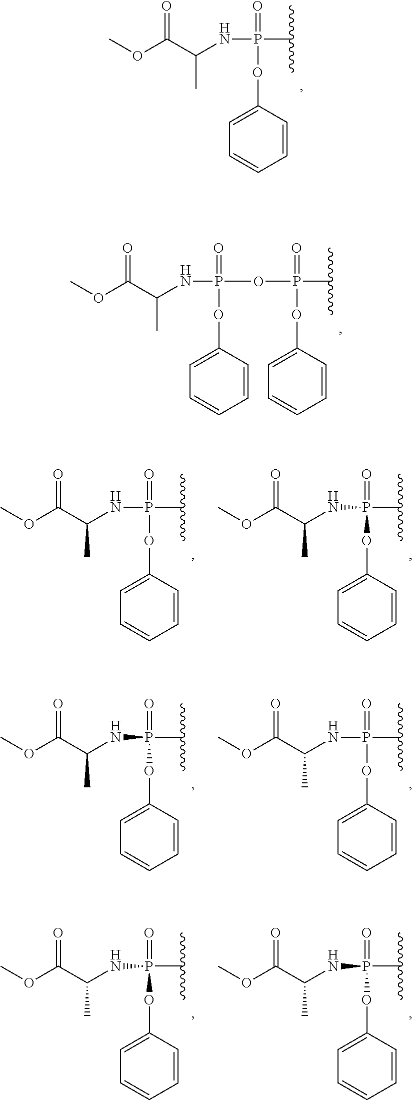

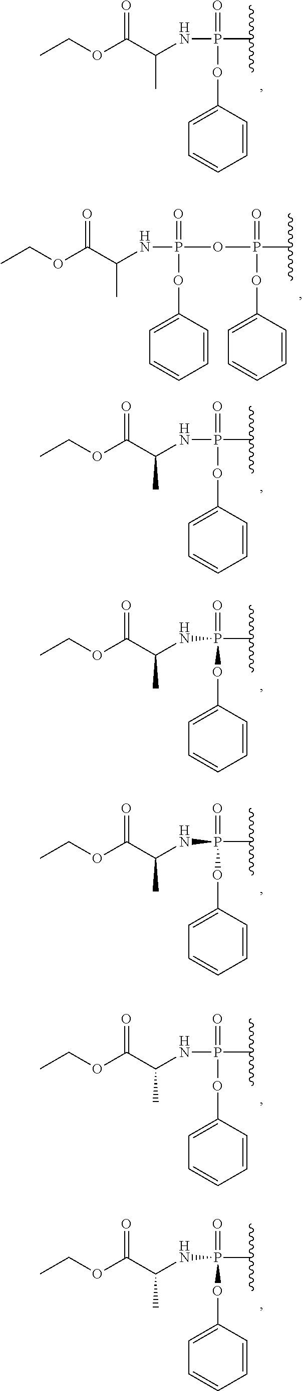

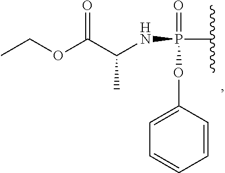 [SMILES: *[P@](=O)(N[C@H](C)C(=O)OCC)Oc1ccccc1]

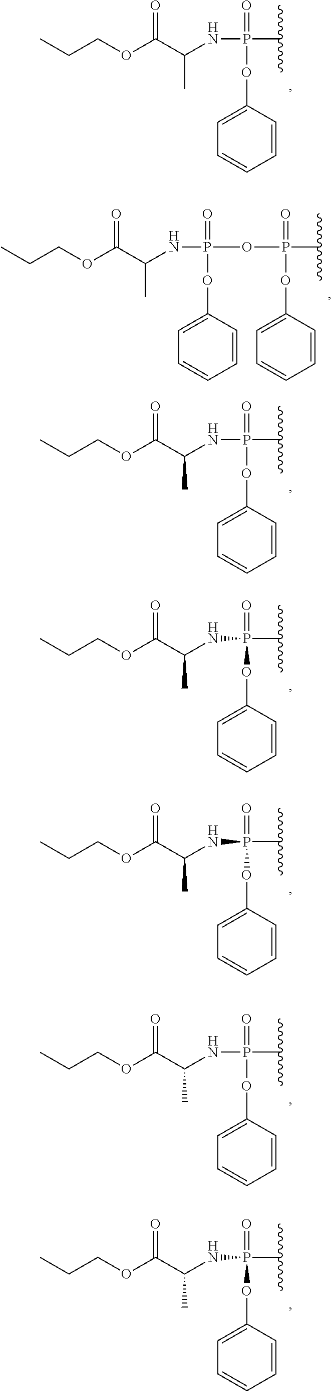

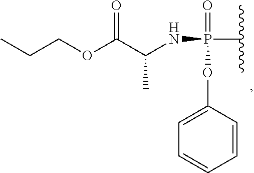 [SMILES: *[P@](=O)(N[C@H](C)C(=O)OCCC)Oc1ccccc1]

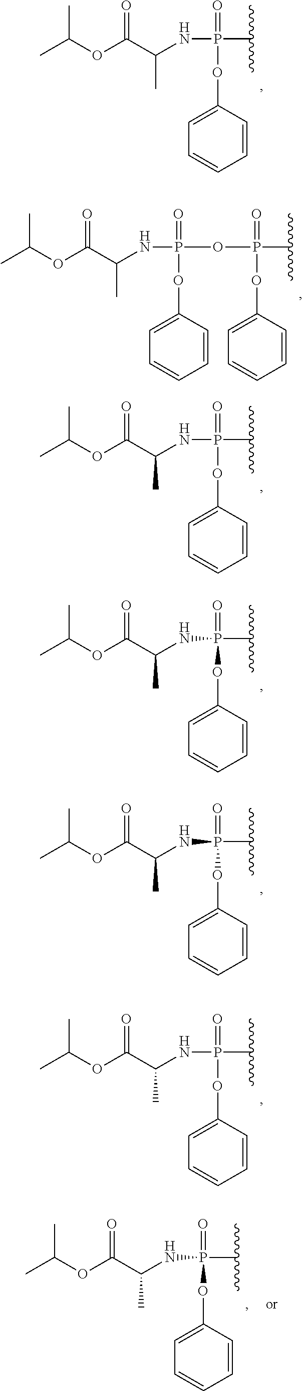

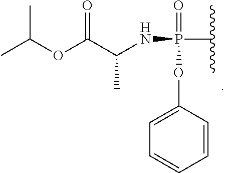 [SMILES: *[P@](=O)(N[C@H](C)C(=O)OC(C)C)Oc1ccccc1]

Aspect 46. The method of claim Aspect 45, wherein R¹ is a group having a structure represented by a formula:

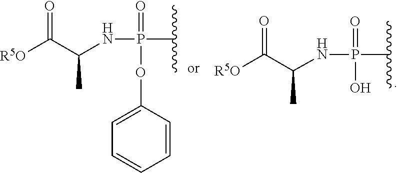

Aspect 47. The method of any one of claims Aspect 44-Aspect 46, wherein R⁵ is selected from lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.
Aspect 48. The method of claim Aspect 47, wherein R⁵ is selected from methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, and cyclohexyl.
Aspect 49. The pharmaceutical composition of claim Aspect 47, wherein R⁵ is selected from methyl, ethyl, propyl, isopropyl, butyl, s-butyl, t-butyl, pentyl, s-pentyl, t-pentyl, neopentyl, 3-pentyl, hexyl, t-hexyl, and 4-septyl.
Aspect 50. The method of claim Aspect 47, wherein R⁵ is selected from methyl, ethyl, propyl, isopropyl, butyl, s-butyl, t-butyl, pentyl, s-pentyl, t-pentyl, neopentyl, and 3-pentyl.
Aspect 51. The method of any one of claim Aspect 44-Aspect 50, wherein the compound is selected from a structure represented by a formula:

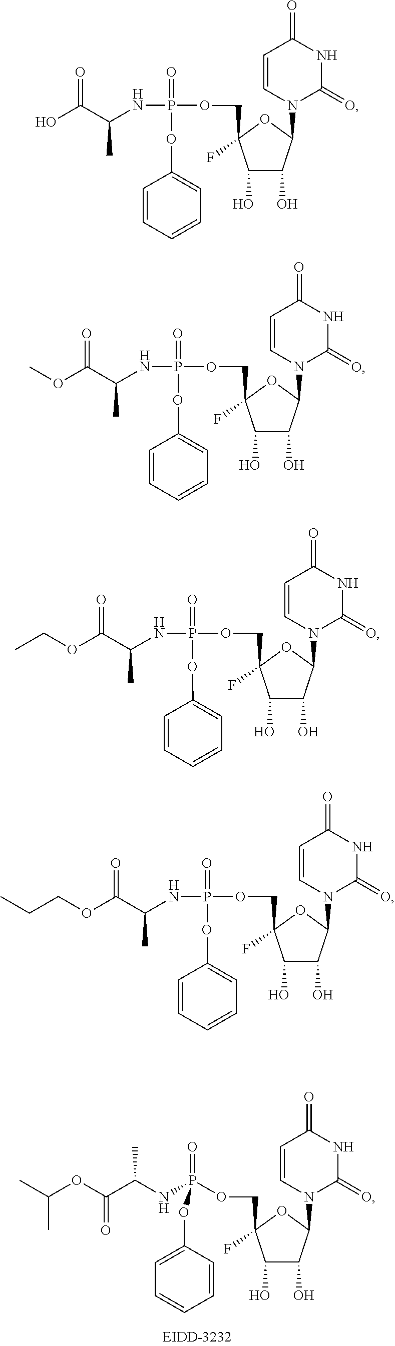 [SMILES: C[C@H](NP(=O)(OC[C@@]1(F)O[C@@H](n2ccc(=O)[nH]c2=O)[C@H](O)[C@@H]1O)Oc1ccccc1)C(=O)O ; COC(=O)[C@H](C)NP(=O)(OC[C@@]1(F)O[C@@H](n2ccc(=O)[nH]c2=O)[C@H](O)[C@@H]1O)Oc1ccccc1 ; CCOC(=O)[C@H](C)NP(=O)(OC[C@@]1(F)O[C@@H](n2ccc(=O)[nH]c2=O)[C@H](O)[C@@H]1O)Oc1ccccc1 ; CCCOC(=O)[C@H](C)NP(=O)(OC[C@@]1(F)O[C@@H](n2ccc(=O)[nH]c2=O)[C@H](O)[C@@H]1O)Oc1ccccc1 ; CC(C)OC(=O)[C@H](C)N[P@](=O)(OC[C@@]1(F)O[C@@H](n2ccc(=O)[nH]c2=O)[C@H](O)[C@@H]1O)Oc1ccccc1]

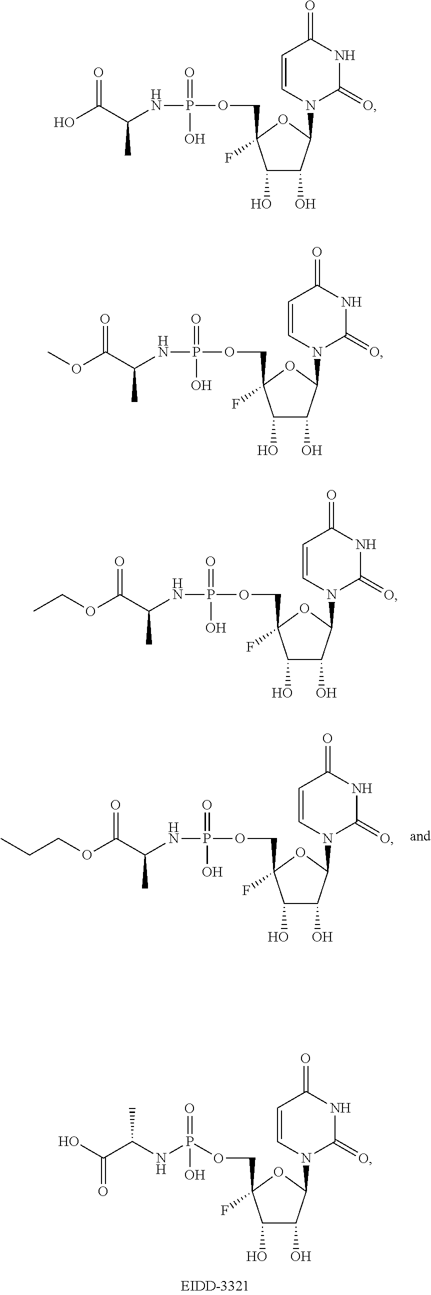

and combinations thereof.
Aspect 52. The method of claim 51, wherein the compound is a structure represented by a formula:

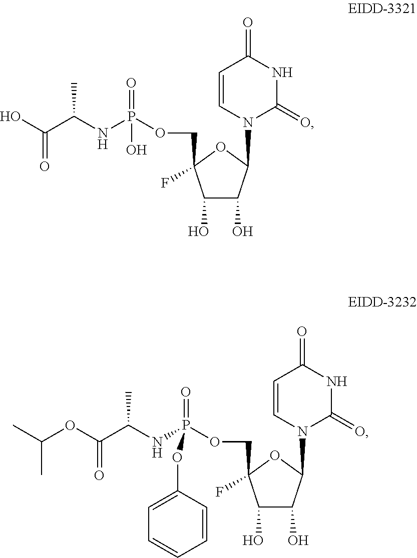 [SMILES: C[C@H](NP(=O)(O)OC[C@@]1(F)O[C@@H](n2ccc(=O)[nH]c2=O)[C@H](O)[C@@H]1O)C(=O)O ; CC(C)OC(=O)[C@H](C)N[P@](=O)(OC[C@@]1(F)O[C@@H](n2ccc(=O)[nH]c2=O)[C@H](O)[C@@H]1O)Oc1ccccc1]

or a combination thereof.
Aspect 53. The method of any one of claims Aspect 44-52, wherein the first treatment period is days 1-5 following diagnosis of the viral infection or presentation for preventing the viral infection.
Aspect 54. The method of claim 53, wherein the first treatment period is days 1-2 following diagnosis of the viral infection or presentation for preventing the viral infection.
Aspect 55. The method of claim 53, wherein the first treatment period is day 1 following diagnosis of the viral infection or presentation for preventing the viral infection.
Aspect 56. The method of any one of claims Aspect 44-55, wherein the loading dose is about 1.1-fold to about 10-fold the treatment dose.
Aspect 57. The method of claim 56, wherein the loading dose is about 1.5-fold to about 5-fold the treatment dose.
Aspect 58. The method of claim Aspect 56, wherein the loading dose is about 1.5-fold to about 2.5-fold the treatment dose.
Aspect 59. The method of any one of claims Aspect 44-58, wherein the loading dose is administered once daily, two times daily, three times daily, or four times daily.
Aspect 60. The method of any one of claims Aspect 44-Aspect 58, wherein the loading dose is administered at least twice daily.
Aspect 61. The method of claim Aspect 59 or claim Aspect 60, wherein the loading dose divided equally among the number of times administered daily.
Aspect 62. A method of producing a drug triphosphate, the method comprising:
providing a plurality of cells;
contacting the plurality of cells with a compound of Formula XXIXc; and
incubating the plurality of cells and the amount of the compound or pharmaceutical composition for period effective to form the drug triphosphate;
wherein the compound of Formula XXIXc is a compound having a structure given by the formula:

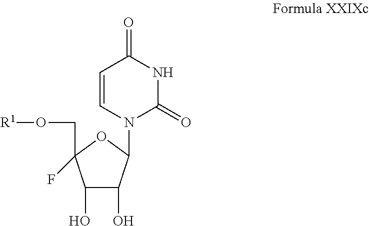

or a pharmaceutical or physiological salt thereof,
wherein R¹ is selected from a group having a structure represented by a formula:

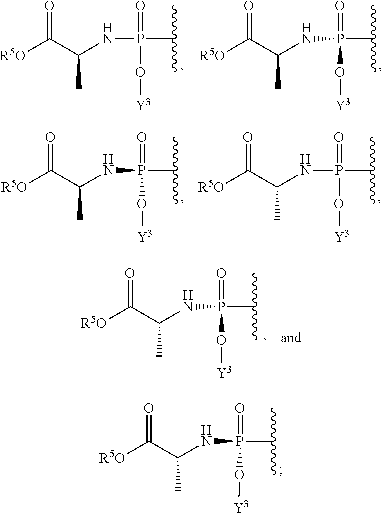

wherein Y³ is hydrogen, aryl, heteroaryl, or heterocyclyl; and wherein Y³ is optionally substituted with one or more, the same or different, R¹⁰;
wherein R⁵ is hydrogen, alkyl, alkenyl, alkynyl, carbocyclyl, heterocarbocyclyl, aryl, heteroaryl, heterocyclyl, cycloalkyl, cycloalkenyl, allenyl, or lipid; and wherein R⁵ is optionally substituted with one or more, the same or different, R¹⁰;
wherein R¹⁰ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl; and wherein R¹⁰ is optionally substituted with one or more, the same or different, R¹¹;
wherein R¹¹ is deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, or carbonyl.
   Aspect 63. The method of claim 62, wherein R¹ is a group having a structure represented by a formula:

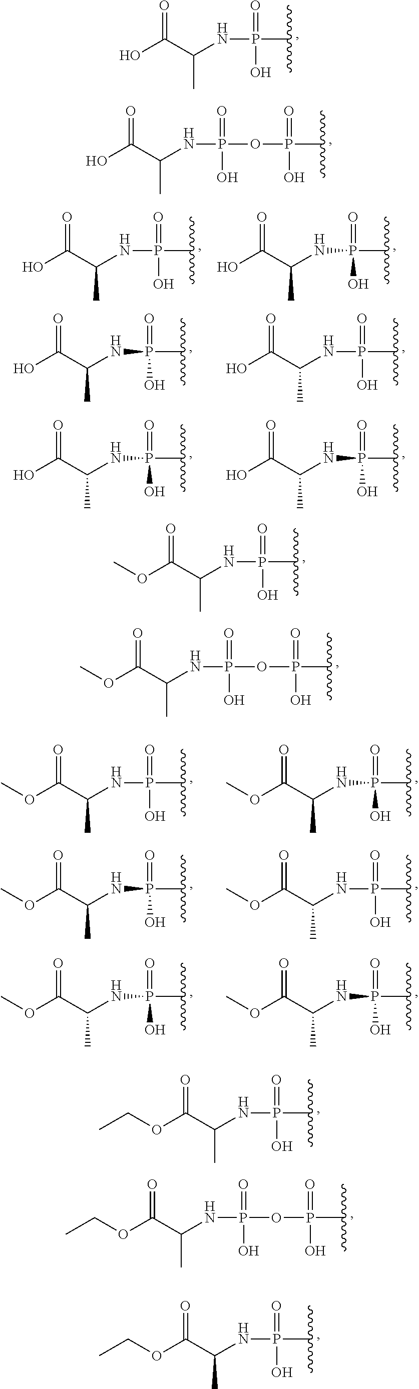

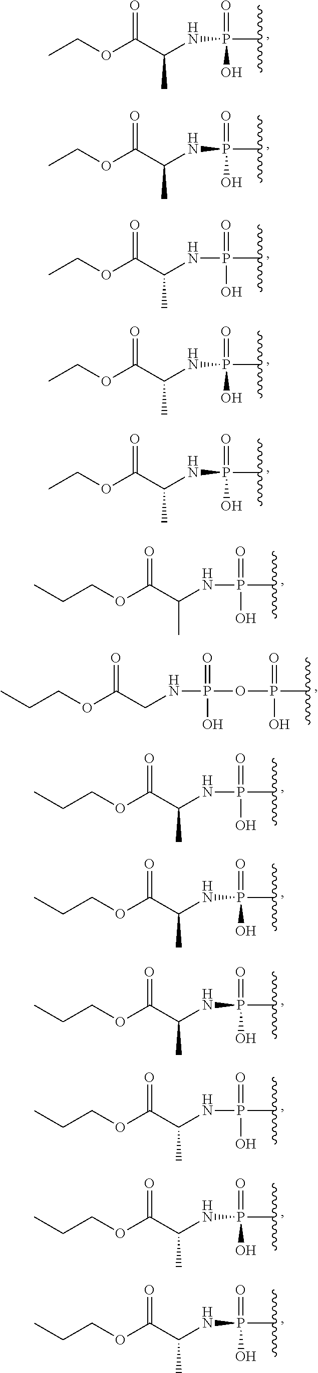

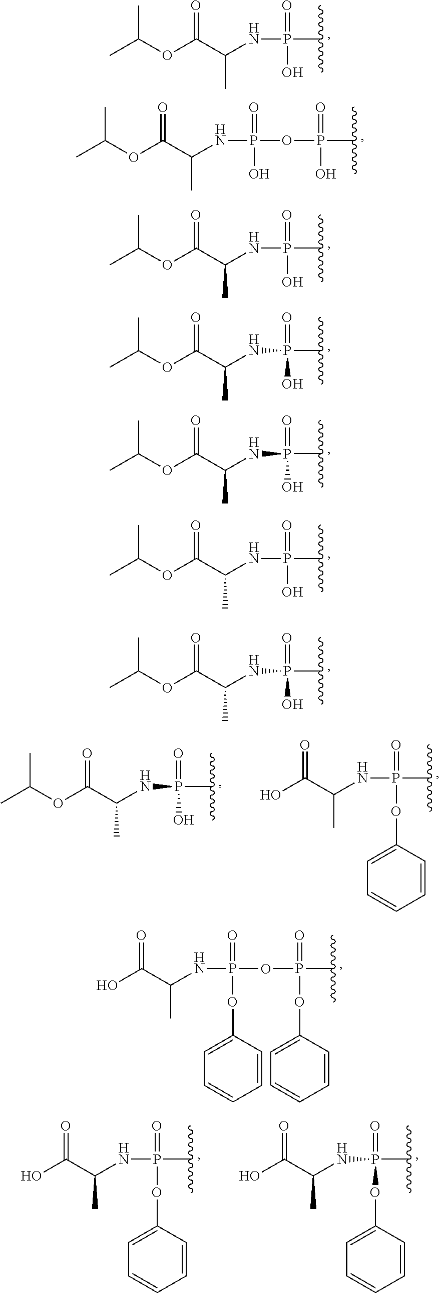

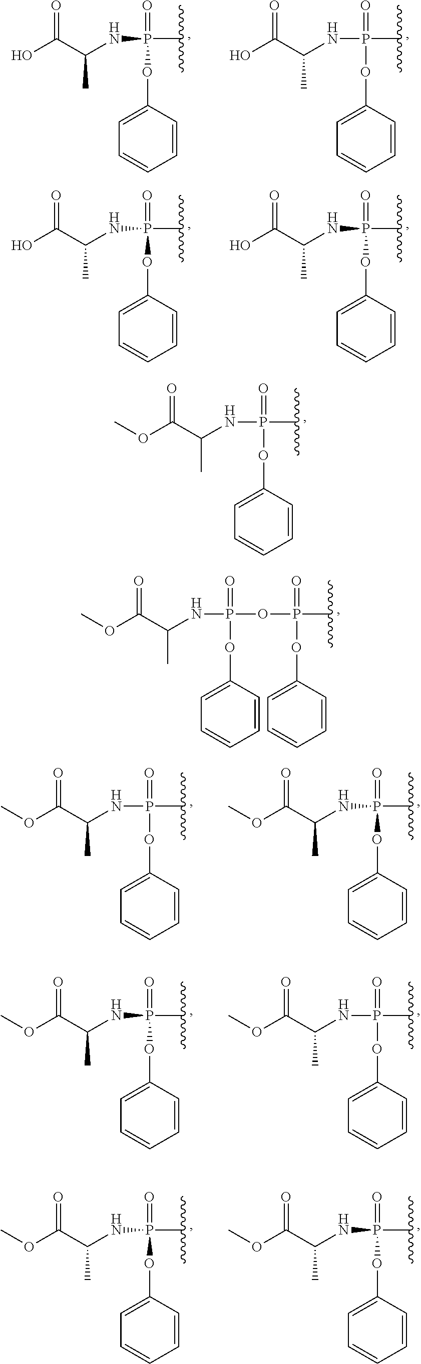

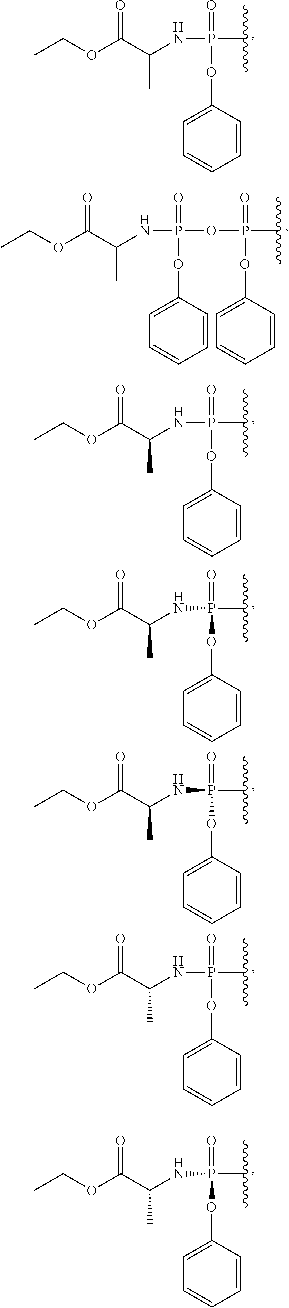

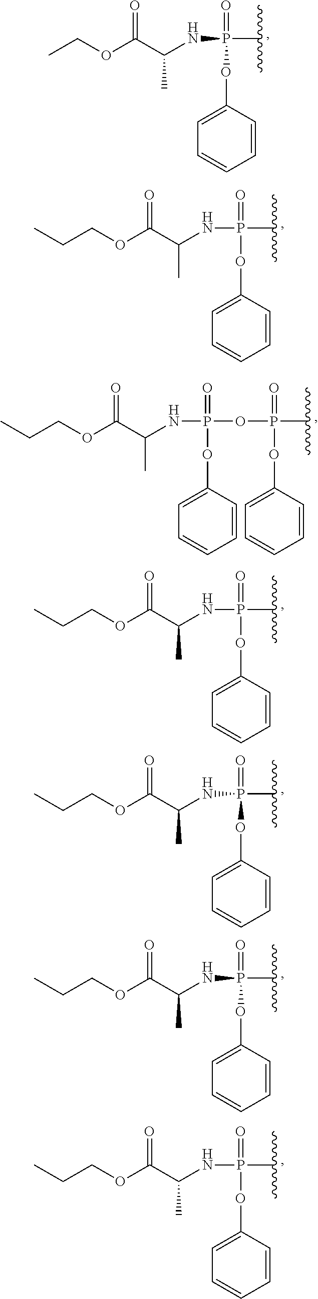

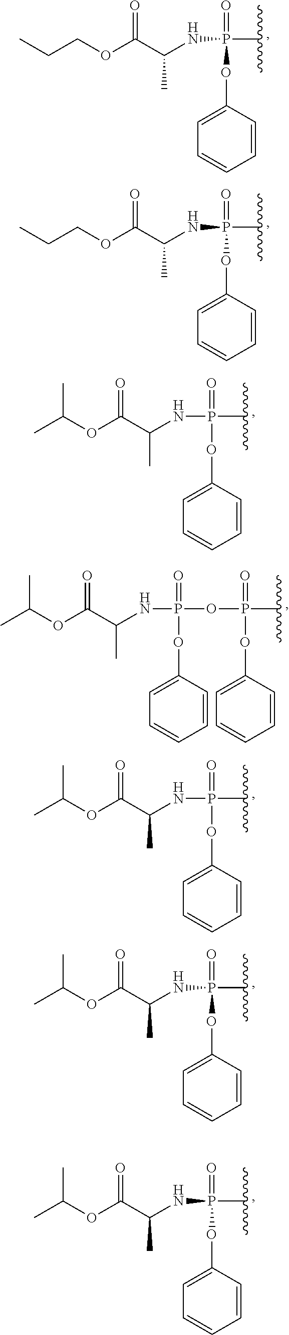

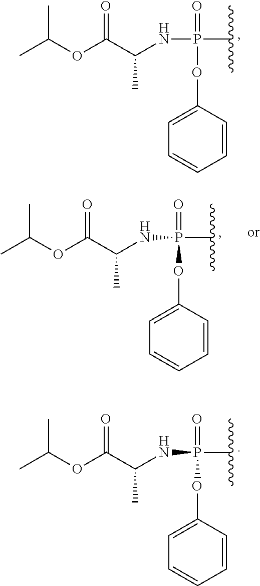

Aspect 64. The method of claim 63, wherein R¹ is a group having a structure represented by a formula:

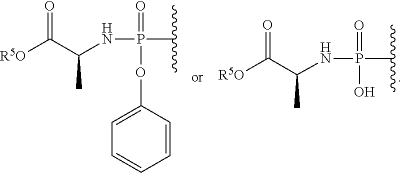

Aspect 65. The method of any one of claims Aspect 62-64, wherein R⁵ is selected from lipid, methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, cyclohexyl, phenyl 2,6-dimethylphenyl, isopropoxide, tert-butoxide, N-propylamino, N-isopropylamino, N-tert-butylamino, N,N-dimethylamino, N,N-diethylamino, and N,N-dipropylamino.
Aspect 66. The method of claim 65, wherein R⁵ is selected from methyl, ethyl, propyl, isopropyl, butyl, s-butyl, t-butyl, pentyl, s-pentyl, t-pentyl, neopentyl, 3-pentyl, hexyl, t-hexyl, 4-septyl, cyclopropyl, cyclobutyl, cyclopentyl, and cyclohexyl.
Aspect 67. The method of claim 66, wherein R⁵ is selected from methyl, ethyl, propyl, isopropyl, butyl, s-butyl, t-butyl, pentyl, s-pentyl, t-pentyl, neopentyl, 3-pentyl, hexyl, t-hexyl, and 4-septyl.
Aspect 68. The method of claim Aspect 66, wherein R⁵ is selected from methyl, ethyl, propyl, isopropyl, butyl, s-butyl, t-butyl, pentyl, s-pentyl, t-pentyl, neopentyl, and 3-pentyl.
Aspect 69. The method of any one of claim Aspect 62-68, wherein the compound is selected from a structure represented by a formula:

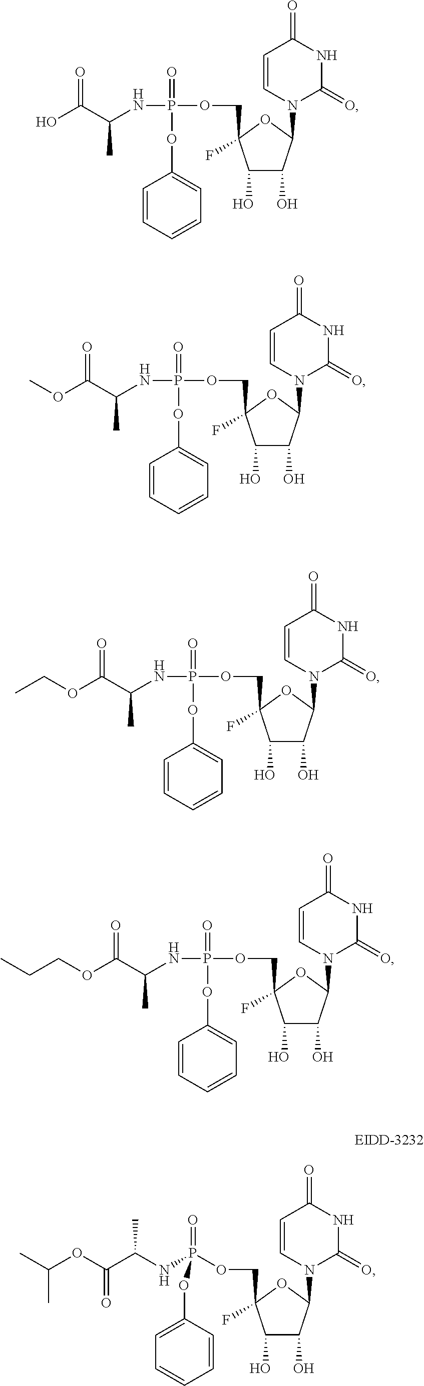 [SMILES: C[C@H](NP(=O)(OC[C@@]1(F)O[C@@H](n2ccc(=O)[nH]c2=O)[C@H](O)[C@@H]1O)Oc1ccccc1)C(=O)O ; COC(=O)[C@H](C)NP(=O)(OC[C@@]1(F)O[C@@H](n2ccc(=O)[nH]c2=O)[C@H](O)[C@@H]1O)Oc1ccccc1 ; CCOC(=O)[C@H](C)NP(=O)(OC[C@@]1(F)O[C@@H](n2ccc(=O)[nH]c2=O)[C@H](O)[C@@H]1O)Oc1ccccc1 ; CCCOC(=O)[C@H](C)NP(=O)(OC[C@@]1(F)O[C@@H](n2ccc(=O)[nH]c2=O)[C@H](O)[C@@H]1O)Oc1ccccc1 ; CC(C)OC(=O)[C@H](C)N[P@](=O)(OC[C@@]1(F)O[C@@H](n2ccc(=O)[nH]c2=O)[C@H](O)[C@@H]1O)Oc1ccccc1]

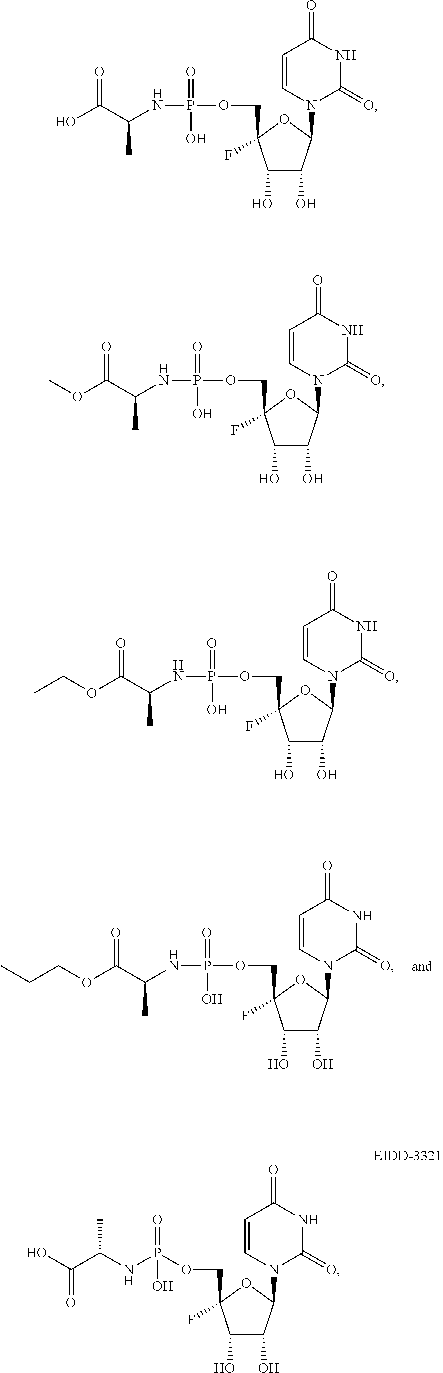 [SMILES: C[C@H](NP(=O)(O)OC[C@@]1(F)O[C@@H](n2ccc(=O)[nH]c2=O)[C@H](O)[C@@H]1O)C(=O)O ; COC(=O)[C@H](C)NP(=O)(O)OC[C@@]1(F)O[C@@H](n2ccc(=O)[nH]c2=O)[C@H](O)[C@@H]1O ; CCOC(=O)[C@H](C)NP(=O)(O)OC[C@@]1(F)O[C@@H](n2ccc(=O)[nH]c2=O)[C@H](O)[C@@H]1O ; CCCOC(=O)[C@H](C)NP(=O)(O)OC[C@@]1(F)O[C@@H](n2ccc(=O)[nH]c2=O)[C@H](O)[C@@H]1O ; C[C@H](NP(=O)(O)OC[C@@]1(F)O[C@@H](n2ccc(=O)[nH]c2=O)[C@H](O)[C@@H]1O)C(=O)O]

and combinations thereof.
Aspect 70. The method of claim 69, wherein the compound is a structure represented by a formula:

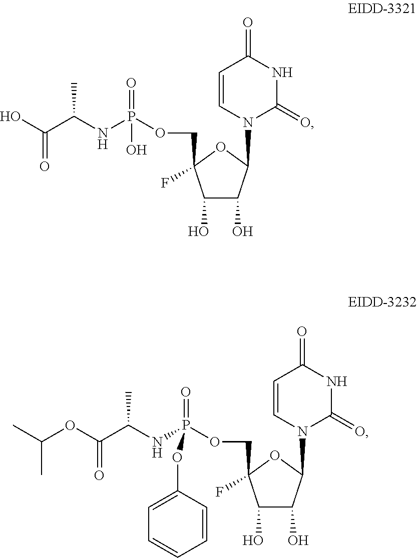 [SMILES: C[C@H](NP(=O)(O)OC[C@@]1(F)O[C@@H](n2ccc(=O)[nH]c2=O)[C@H](O)[C@@H]1O)C(=O)O ; CC(C)OC(=O)[C@H](C)N[P@](=O)(OC[C@@]1(F)O[C@@H](n2ccc(=O)[nH]c2=O)[C@H](O)[C@@H]1O)Oc1ccccc1]

or a combination thereof.
Aspect 71. The method of any one of claims Aspect 62-70, wherein the plurality of cells are in vivo.
Aspect 72. The method of any one of claims claim Aspect 62-71, wherein the contacting is administering to a subject in need thereof.
Aspect 73. A compound having a structure represented by a formula:

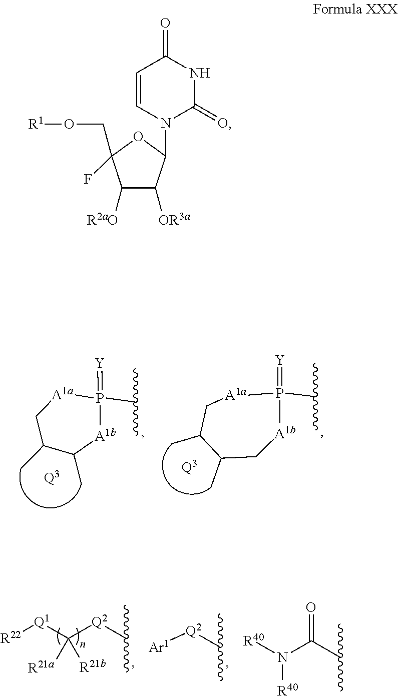

wherein Q¹ is null, O, S, C(═O), O(C═O), S(C═O), NR⁴⁰(C═O), C(═S), O(C═S), S(C═S), NR⁴⁰(C═S), O(C═O)O, S(C═O)O, NR⁴⁰(C═O)O, S(C═O)S, NR⁴⁰(C═O)S, NR⁴⁰(C═O)NR⁴⁰, or O(C═S)O, S(C═S)O, NR⁴⁰(C═S)O, S(C═S)S, NR⁴⁰(C═S)S, NR⁴⁰(C═S)NR⁴⁰, or NR⁴⁰,
Q¹ is C(═O), O(C═O), S(C═O), NR⁴⁰(C═O), C(═S), O(C═S), S(C═S), or NR⁴⁰(C═S),
Q³ represents a C₅-C₈carbocyclyl, C₆-C₁₂aryl, C₃-C₁₂heterocyclyl, or C₃-C₁₂heteroaryl;
wherein Y is O or S;
wherein n is selected from 1, 2, and 3, preferably 1;
wherein each of A^{1a}, A^{1b}, A^{1c}, and A^{1d} are independently selected from C, NR⁴⁰, S, and O;
wherein R⁴⁰ is in each case independently selected from hydrogen and C₁-C₆alkyl,
optionally substituted one more times by R¹⁰;
provided that Q³ represents a phenyl ring, then A^{1a} and A^{1b} cannot both be O;
wherein Ar¹ is selected from a 3-12 membered carbocycle, 3-12 membered heterocycle, a 6-12 membered aryl, and a 5-12 heteroaryl; wherein Ar¹ can be optionally independently substituted with one or more, the same or different, R¹⁰;
wherein each of R^{2a} and R^{3a} is independently selected from deuterium, hydrogen, C₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, polyethylene glycol, aryl optionally substituted with an alkyl group, and lipid;
wherein R^{2a} and R^{3a} can each be optionally independently substituted with one or more, the same or different, R¹⁰; and
wherein R^{2a} and R^{3a} can together form a 5-7 membered heterocyclic ring, for example R^{2a} and R^{3a} can together C(CH₃)₂, C(═O) or C(═S);
wherein R¹⁰ is deuterium, hydroxy, azido, thiol, amino, cyano, halogen, alkyl, or alkenyl;
wherein lipid is a C₁-C₂₂ higher alkyl, C₁-C₂₂ higher alkoxy, or a lipid as described herein;
wherein each of R^{21a}, R^{21a}, and R²² is independently selected from hydrogen, deuterium, C₁-C₁₂ alkyl, C₁-C₁₂ alkylamino, (C₁-C₁₂ alkyl)₂amino, (CH₂)_{q}—(C₁-C₁₂ alkoxy), C₃-C₁₂cycloalkyl, C₃-C₁₂heterocyclyl, and C₁-C₁₂ alkoxy;
wherein q is an integer selected from 1, 2, and 3;
wherein each of R^{21a}, R^{21b}, and R²² can be optionally substituted with one or more, the same or different, R¹⁰;
wherein R^{21a} and R^{21b} are optionally covalently bonded, and together with the intermediate atoms, comprise an optionally substituted 3- to 7-membered spirocycloalkyl or spiroheterocyclyl;
wherein R²² is optionally covalently bonded to R^{21a} or R^{21b}, and together with the intermediate atoms, comprise an optionally substituted 3- to 7-membered cycloalkyl or heterocyclyl;
wherein R²² can be optionally substituted with one or more, the same or different, R¹⁰;
wherein R¹⁰ is in each case independently alkynyl, alkoxy, amino, alkylamino, (alkyl)₂amino, aryl, heteroaryl, allenyl, sulfinyl, sulfamoyl, sulfonyl, polyethylene glycol, lipid, nitro, or carbonyl, wherein R¹⁰ is optionally independently substituted with one or more, the same or different, R¹¹;
wherein R¹¹ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, or oxo;
or a pharmaceutically acceptable salt, solvate, or polymorph thereof.
   Aspect 74. The compound of claim 73, wherein R¹ is a structure represented by a formula selected from:

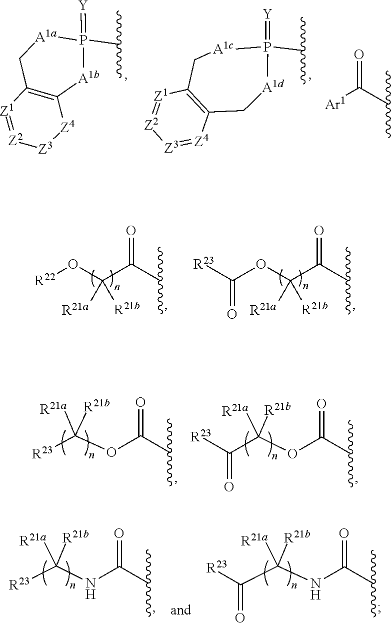

wherein n is selected from 1, 2, and 3;
wherein Y is O or S;
wherein Z¹ is selected from N and C—R^{20a};
wherein Z² is selected from N and C—R^{20b};
wherein Z³ is selected from N and C—R^{20c};
wherein Z⁴ is selected from N and C—R^{20d};
   provided that no more than three of Z¹, Z², Z³, and Z⁴ are N;
wherein each of A^{1a}, A^{1b}, A^{1c}, and A^{1d} are independently selected from C, NR⁴⁰, S, and O;
   wherein is R⁴⁰ selected from hydrogen and C₁-C₆;
   provided that if at least one of Z¹, Z², Z³, and Z⁴ is not N, then A^{1a} and Alb cannot both be O;
wherein Ar¹ is selected from a 3-12 membered carbocycle, 3-12 membered heterocarbocycle, a 5-12 membered aryl, and a 5-12 heteroaryl;
wherein each of R^{2a} and R^{3a} is independently selected from deuterium, hydrogen, —Ac, C₁-C₆ alkyl, C₁-C₆ carboxamide, C₁-C₆ carboxylate, polyethylene glycol, aryl substituted with an alkyl group, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 3- to 7-membered cycloalkyl or cycloheteroalkyl;
wherein R^{20a}, R^{20b}, R^{20c}, and R^{20d} are each independently selected from hydrogen, deuterium, hydroxyl, amino, cyano, halogen, —OAc, C₁-C₃ alkyl, C₁-C₃ alkoxy, C₁-C₃ alkylamino, (C₁-C₃ alkyl)₂amino, C₁-C₃ carboxamide, and lipid;
wherein R^{20a}, R^{20b}, R^{20c}, and R^{20d} can each be optionally independently substituted with one or more, the same or different, R¹⁰;
wherein R¹⁰ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, or carbonyl, wherein R¹⁰ is optionally independently substituted with one or more, the same or different, R¹¹;
wherein R¹¹ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, or carbonyl;
wherein lipid is a C₁₁-C₂₂ higher alkyl, C₁₁-C₂₂ higher alkoxy, or a lipid as described herein;
wherein each of R^{21a}, R^{21b}, and R²³ is independently selected from hydrogen, deuterium, C₁-C₁₂ alkyl, C₁-C₁₂ alkylamino, (C₁-C₁₂ alkyl)₂amino, (CH₂)_{q}—(C₁-C₁₂ alkoxy), and C₁-C₁₂ alkoxy;
   wherein q is an integer selected from 1, 2, and 3;
   wherein each of R^{21a}, R^{21b}, and R²³ can be optionally substituted with one or more, the same or different, R¹⁰;
   wherein R^{21a} and R^{21b} are optionally covalently bonded, and together with the intermediate atoms, comprise an optionally substituted 5- to 7-membered spirocycloalkyl or spirocycloheteroalkyl;
   wherein R²³ is optionally covalently bonded to R^{21a} or R^{21b}, and together with the intermediate atoms, comprise an optionally substituted 5- to 7-membered cycloalkyl or heterocycloalkyl;
wherein R²² is selected from hydrogen, deuterium, and C₁-C₂₂ alkyl;
   wherein R²² can be optionally substituted with one or more, the same or different, R¹⁰; and
   wherein R²² is optionally covalently bonded to R^{21a} or R^{21b}, and together with the intermediate atoms, comprise an optionally substituted 3- to 7-membered cycloalkyl or heterocycloalkyl.
      Aspect 75. The compound of claim Aspect 73 or Aspect 74, wherein the compound has a structure represented by a formula selected from:

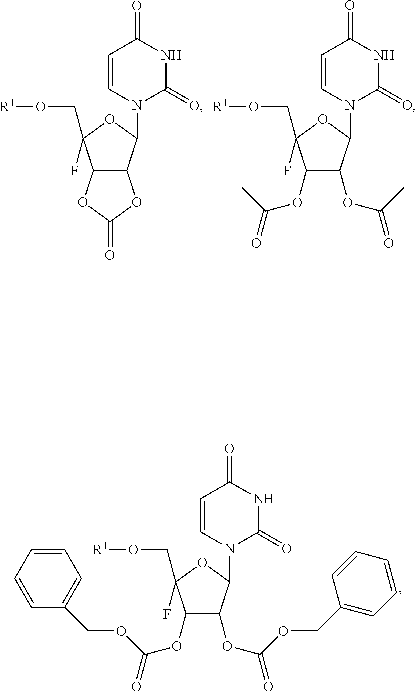

and combinations thereof.
Aspect 76. The compound of claim Aspect 73 or Aspect 74, wherein the compound has a structure represented by a formula:

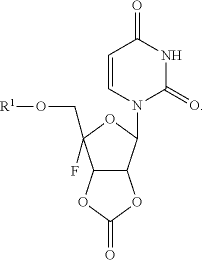

Aspect 77. The compound of claim Aspect 73 or Aspect 74, wherein the compound has a structure represented by a formula:

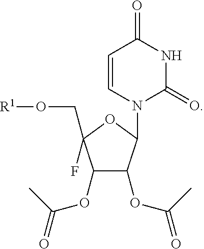

Aspect 78. The compound of claim Aspect 73 or Aspect 74, wherein the compound has a structure represented by a formula:

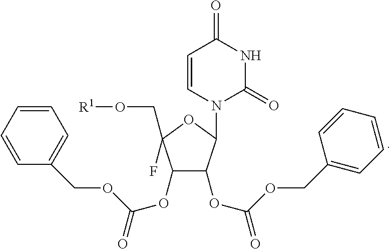

Aspect 79. The compound of any one of claims Aspect 73-Aspect 78, wherein R¹ is a structure represented by a formula selected from:

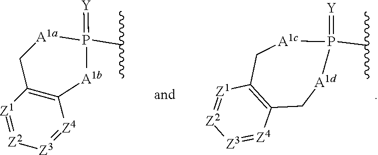

Aspect 80. The compound of claim Aspect 79, wherein R¹ is a structure represented by a formula:

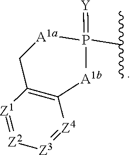

Aspect 81. The compound of claim Aspect 80, wherein R¹ is a structure represented by a formula selected from:

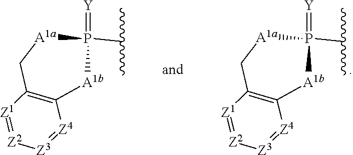

Aspect 82. The compound of claim Aspect 79, wherein R¹ is a structure represented by a formula:

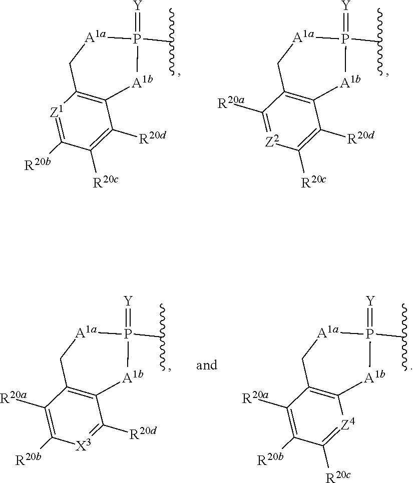

Aspect 83. The compound of claim Aspect 82, wherein R¹ is a structure represented by a formula:

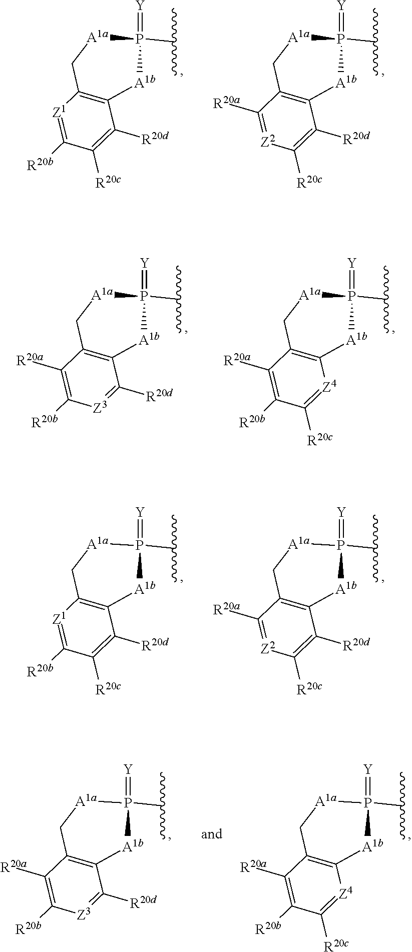

Aspect 84. The compound of claim Aspect 79, wherein R¹ is a structure represented by a

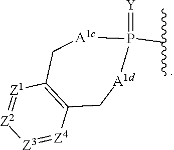

Aspect 85. The compound of any one of claims Aspect 73-Aspect 84, wherein the compound has a structure represented by a formula:

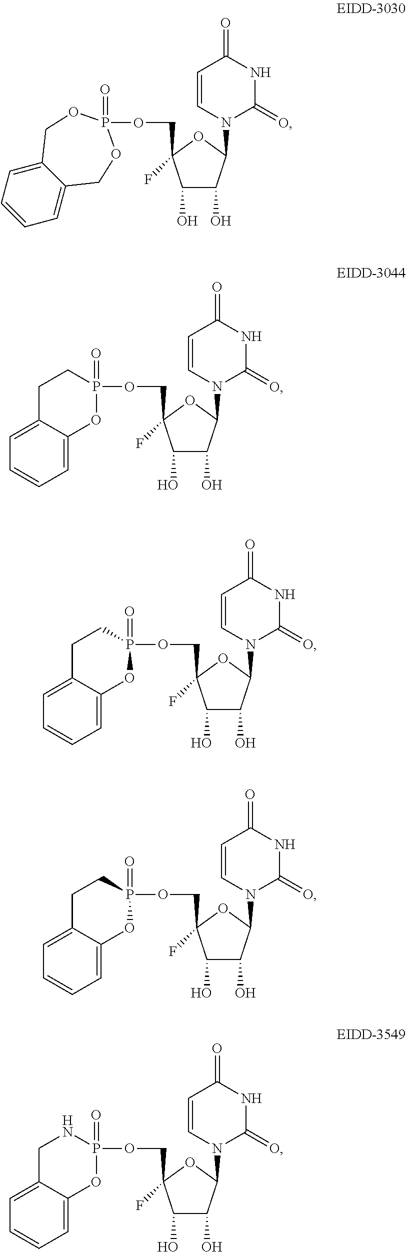 [SMILES: O=c1ccn([C@@H]2O[C@](F)(COP3(=O)OCc4ccccc4CO3)[C@@H](O)[C@H]2O)c(=O)[nH]1 ; O=c1ccn([C@@H]2O[C@](F)(COP3(=O)CCc4ccccc4O3)[C@@H](O)[C@H]2O)c(=O)[nH]1 ; O=c1ccn([C@@H]2O[C@](F)(CO[P@@]3(=O)CCc4ccccc4O3)[C@@H](O)[C@H]2O)c(=O)[nH]1 ; O=c1ccn([C@@H]2O[C@](F)(CO[P@]3(=O)CCc4ccccc4O3)[C@@H](O)[C@H]2O)c(=O)[nH]1 ; O=c1ccn([C@@H]2O[C@](F)(COP3(=O)NCc4ccccc4O3)[C@@H](O)[C@H]2O)c(=O)[nH]1]

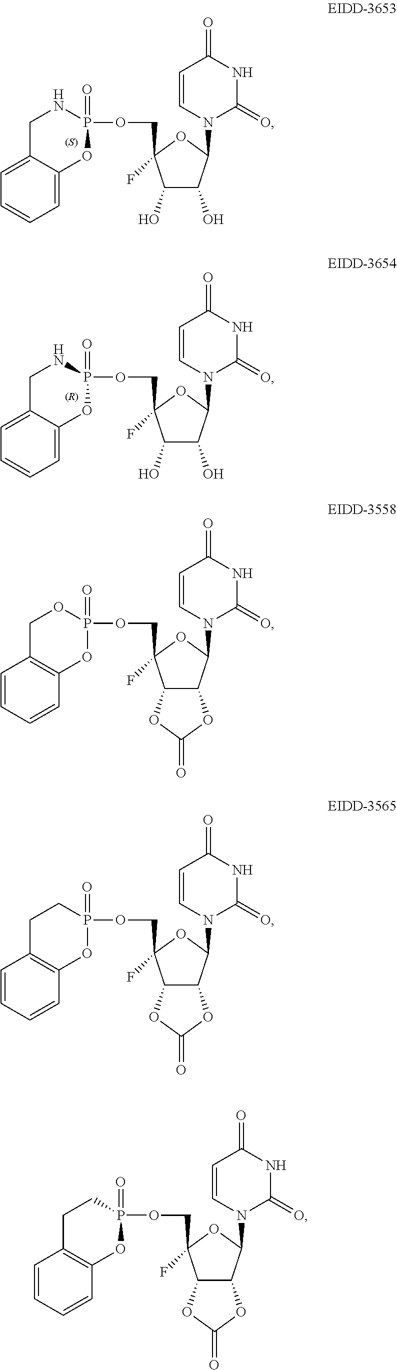

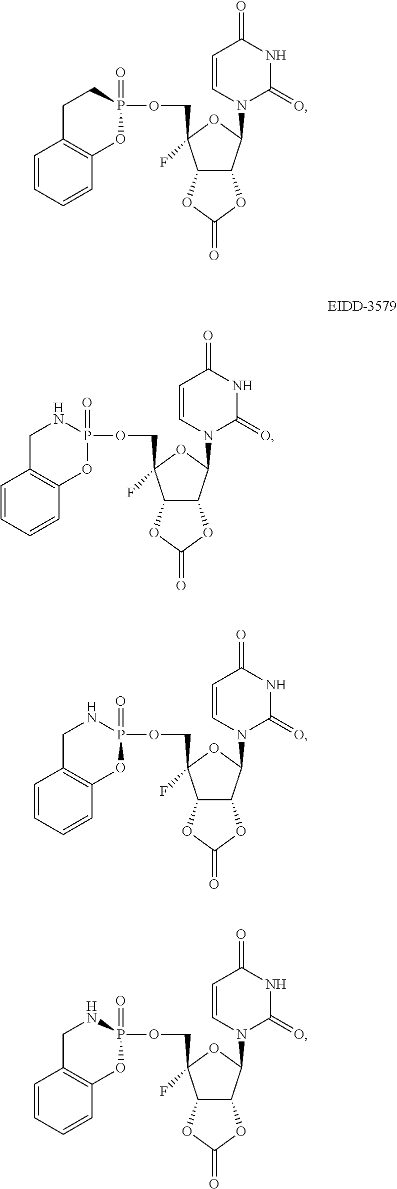 [SMILES: O=C1O[C@H]2[C@H](n3ccc(=O)[nH]c3=O)O[C@](F)(CO[P@]3(=O)CCc4ccccc4O3)[C@H]2O1 ; O=C1O[C@H]2[C@H](n3ccc(=O)[nH]c3=O)O[C@](F)(COP3(=O)NCc4ccccc4O3)[C@H]2O1 ; O=C1O[C@H]2[C@H](n3ccc(=O)[nH]c3=O)O[C@](F)(CO[P@@]3(=O)NCc4ccccc4O3)[C@H]2O1 ; O=C1O[C@H]2[C@H](n3ccc(=O)[nH]c3=O)O[C@](F)(CO[P@]3(=O)NCc4ccccc4O3)[C@H]2O1]

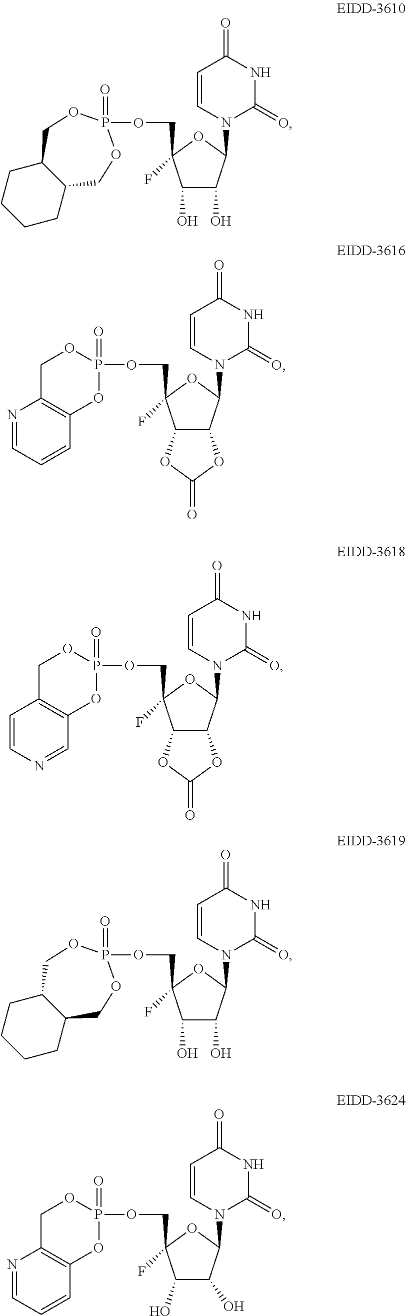

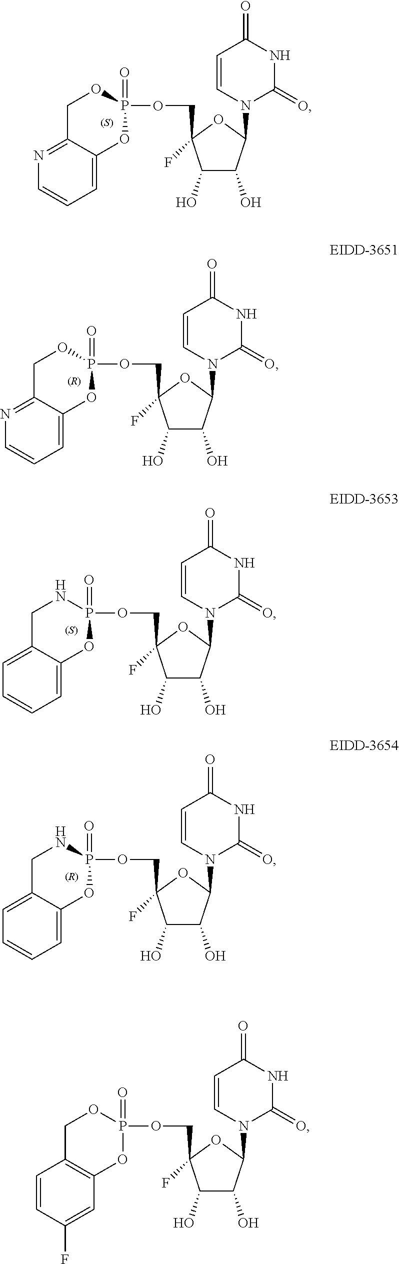

or combinations thereof
Aspect 86. The compound of any one of claims Aspect 73-Aspect 78, wherein Ar¹ has a structure represented by a formula selected from:

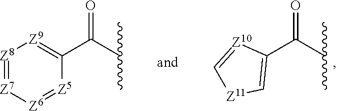

wherein Z⁵ is selected from N and C—R^{20e};
wherein Z⁶ is selected from N and C—R^{20f};
wherein Z⁷ is selected from N and C—R^{20g};
wherein Z⁸ is selected from N and C—R^{20h}.
wherein Z⁹ is selected from N and C—R²⁰ⁱ;
provided that no more than three of Z⁵, Z⁶, Z⁷, Z⁸, and Z⁹ are N;
wherein R^{20e}, R^{20f}, R^{20g}, R^{20h}, and R²⁰ⁱ are each independently selected from hydrogen, deuterium, hydroxyl, amino, cyano, halogen, —OAc, C₁-C₃ alkyl, C₁-C₃ alkoxy, C₁-C₃ alkylamino, (C₁-C₃ alkyl)₂amino, C₁-C₃ carboxamide, and lipid;
wherein R^{20e}, R^{20f}, R^{20g}, R^{20h}, and R²⁰ⁱ can each be optionally independently substituted with one or more, the same or different, R¹⁰;
wherein two of R^{20e}, R^{20f}, R^{20g}, R^{20h}, and R²⁰ⁱ are optionally covalently bonded, and together with the intermediate atoms, comprise an optionally substituted 3- to 7-membered cycloalkyl or cycloheteroalkyl;
wherein Z¹⁰ is selected from N and C—R^{20j};
wherein Z¹¹ is selected from N—R^{20k} and C—R^{20l}R^{20m}.
wherein R^{20j}, R^{20l}, and R^{20m} are each independently selected from hydrogen, deuterium, hydroxyl, amino, cyano, halogen, —OAc, C₁-C₃ alkyl, C₁-C₃ alkoxy, C₁-C₃ alkylamino, (C₁-C₃ alkyl)₂amino, C₁-C₃ carboxamide and lipid;
wherein R^{20j}, R^{20l}, and R^{20m} can each be optionally independently substituted with one or more, the same or different, R¹⁰; and
wherein R^{20k} is selected from hydrogen, deuterium, —OAc, C₁-C₃ alkyl, C₁-C₃ alkoxy, C₁-C₃ alkylamino, (C₁-C₃ alkyl)₂amino, and C₁-C₃ carboxamide.
   Aspect 87. The compound of any one of claims Aspect 73-Aspect 78, wherein R¹ has a structure represented by a formula:

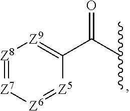

wherein Z⁵ is selected from N and C—R^{20e};
wherein Z⁶ is selected from N and C—R^{20f};
wherein Z⁷ is selected from N and C—R^{20g};
wherein Z⁸ is selected from N and C—R^{20h};
wherein Z⁹ is selected from N and C—R²⁰ⁱ;
provided that no more than three of Z⁵, Z⁶, Z⁷, Z⁸, and Z⁹ are N;
wherein R^{20e}, R^{20f}, R^{20g}, R^{20h}, and R²⁰ⁱ are each independently selected from hydrogen, deuterium, hydroxyl, amino, cyano, halogen, —OAc, C₁-C₃ alkyl, C₁-C₃ alkoxy, C₁-C₃ alkylamino, (C₁-C₃ alkyl)₂amino, C₁-C₃ carboxamide and lipid;
wherein R^{20e}, R^{20f}, R^{20g}, R^{20h}, and R²⁰ⁱ can each be optionally independently substituted with one or more, the same or different, R¹⁰; and wherein two of R^{20e}, R^{20f}, R^{20g}, R^{20h}, and R²⁰ⁱ are optionally covalently bonded, and together with the intermediate atoms, comprise an optionally substituted 5- to 7-membered cycloalkyl or cycloheteroalkyl.
   Aspect 88. The compound of claim Aspect 87, wherein the compound has a structure represented by a formula selected from:

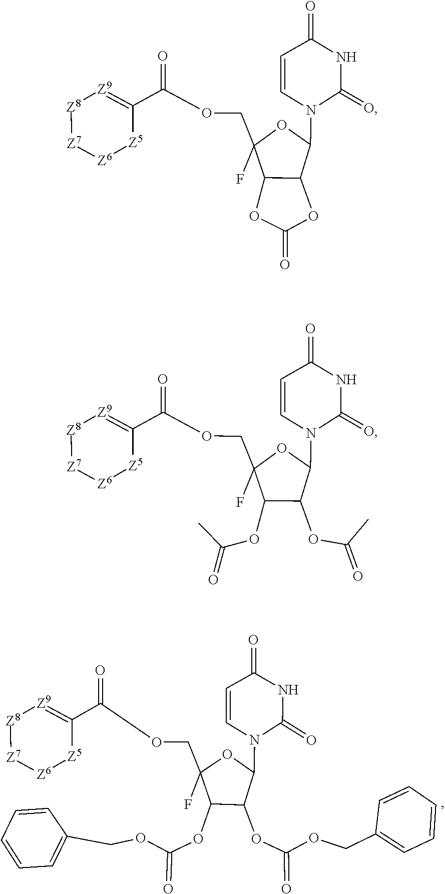

and combinations thereof.
Aspect 89. The compound of claim Aspect 87, wherein the compound has a structure represented by a formula:

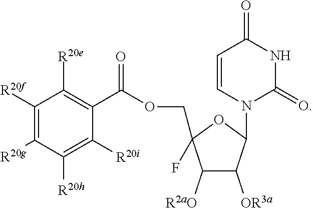

Aspect 90. The compound of claim Aspect 89, wherein the compound is selected from a structure represented by a formula selected from:

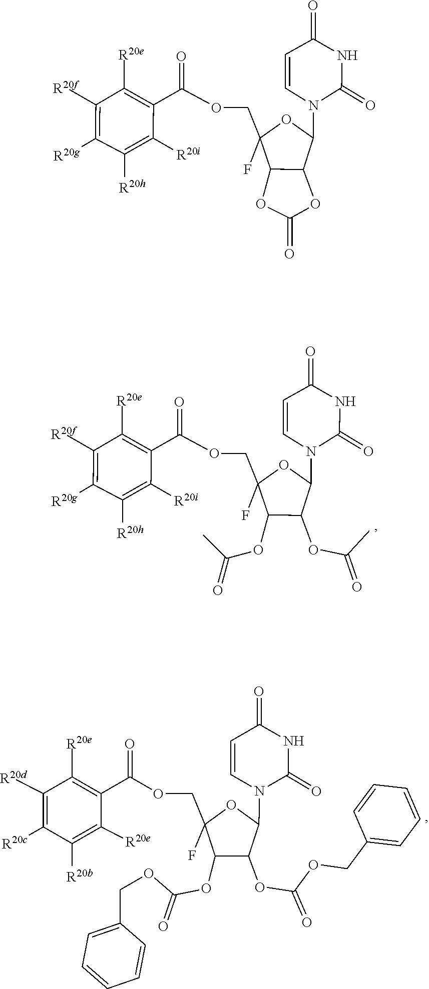

and combinations thereof
Aspect 91. The compound of any one of claims Aspect 87-Aspect 90, wherein at least one of R^{20e}, R^{20f}, R^{20g}, R^{20h}, and R²⁰ⁱ is not hydrogen.
Aspect 92. The compound of any one of claims Aspect 87-Aspect 91, wherein R^{20e}, R^{20f}, R^{20g}, R^{20h}, and R²⁰ⁱ are each independently selected from hydrogen, deuterium, hydroxyl, amino, cyano, halogen, and combinations thereof.
Aspect 93. The compound of claim Aspect 92, wherein R^{20e}, R^{20f}, R^{20g}, R^{20h}, and R²⁰ⁱ are each independently selected from hydrogen, halogen, and combinations thereof.
Aspect 94. The compound of claim Aspect 93, wherein halogen is selected from —Cl, —F, and —I.
Aspect 95. The compound of claim Aspect 94, wherein halogen is —Cl.
Aspect 96. The compound of any one of claims Aspect 87-Aspect 95, wherein the compound is selected from a structure having a formula:

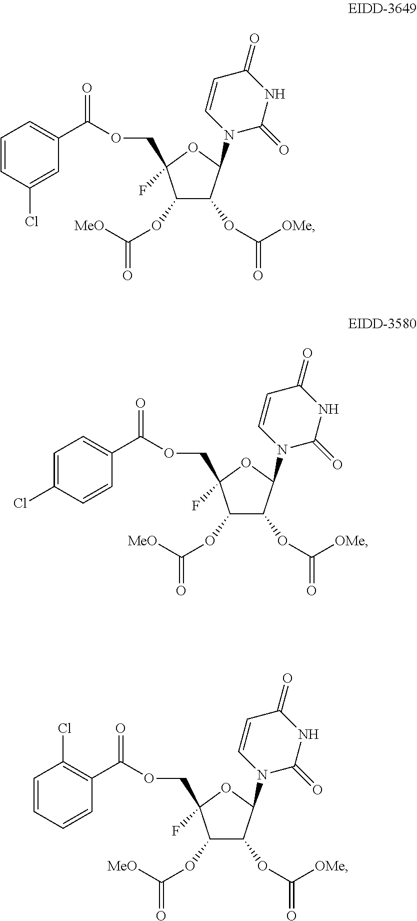 [SMILES: COC(=O)O[C@H]1[C@H](n2ccc(=O)[nH]c2=O)O[C@](F)(COC(=O)c2cccc(Cl)c2)[C@H]1OC(=O)OC ; COC(=O)O[C@H]1[C@H](n2ccc(=O)[nH]c2=O)O[C@](F)(COC(=O)c2ccc(Cl)cc2)[C@H]1OC(=O)OC ; COC(=O)O[C@H]1[C@H](n2ccc(=O)[nH]c2=O)O[C@](F)(COC(=O)c2ccccc2Cl)[C@H]1OC(=O)OC]

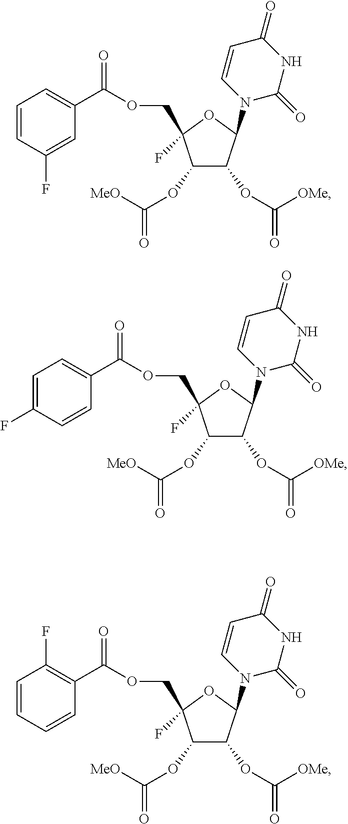

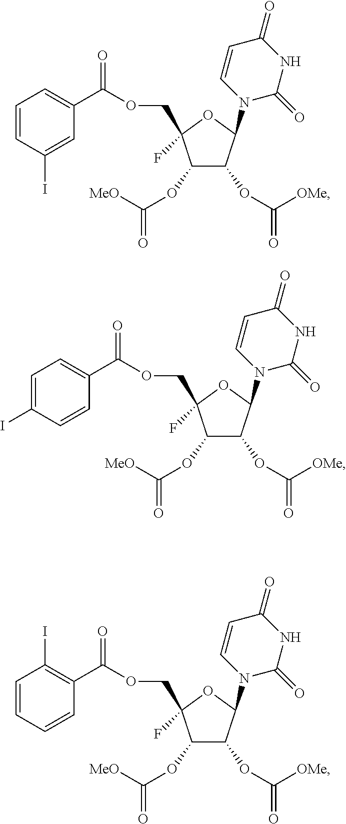

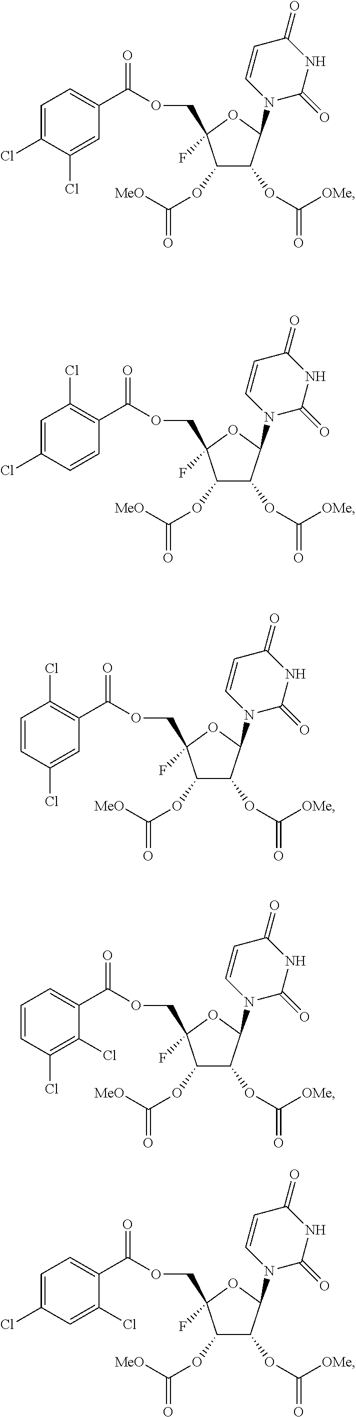

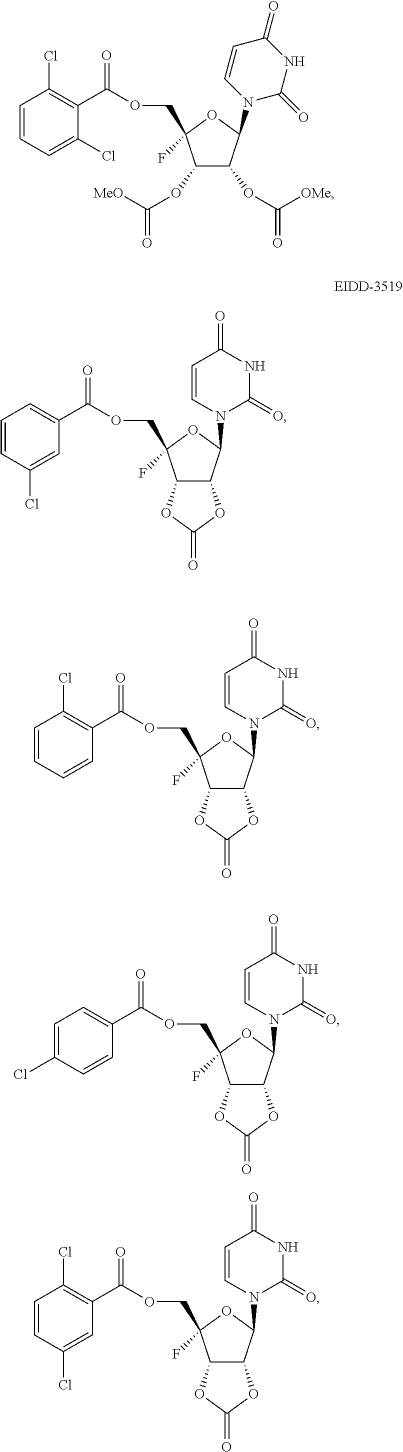

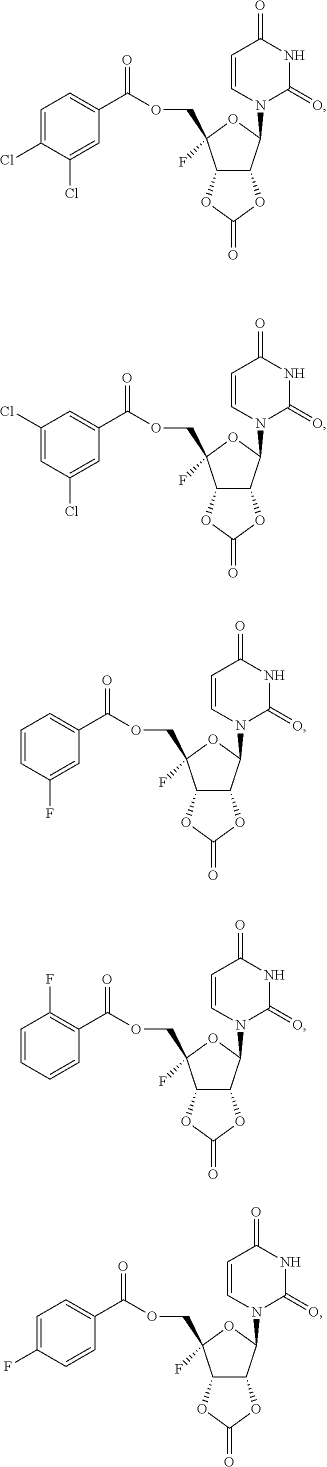 [SMILES: O=C1O[C@H]2[C@H](n3ccc(=O)[nH]c3=O)O[C@](F)(COC(=O)c3ccc(Cl)c(Cl)c3)[C@H]2O1 ; O=C1O[C@H]2[C@H](n3ccc(=O)[nH]c3=O)O[C@](F)(COC(=O)c3cc(Cl)cc(Cl)c3)[C@H]2O1 ; O=C1O[C@H]2[C@H](n3ccc(=O)[nH]c3=O)O[C@](F)(COC(=O)c3cccc(F)c3)[C@H]2O1 ; O=C1O[C@H]2[C@H](n3ccc(=O)[nH]c3=O)O[C@](F)(COC(=O)c3ccccc3F)[C@H]2O1 ; O=C1O[C@H]2[C@H](n3ccc(=O)[nH]c3=O)O[C@](F)(COC(=O)c3ccc(F)cc3)[C@H]2O1]

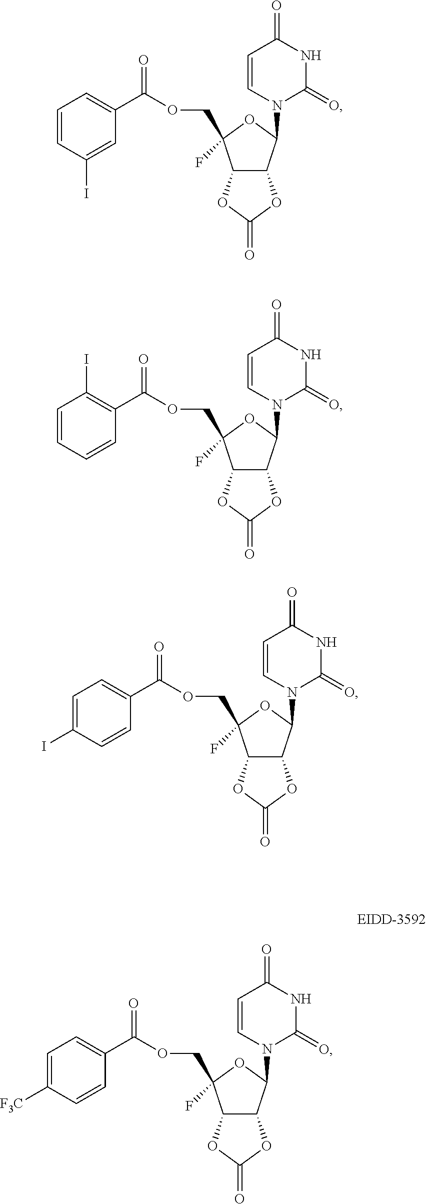 [SMILES: O=C1O[C@H]2[C@H](n3ccc(=O)[nH]c3=O)O[C@](F)(COC(=O)c3cccc(I)c3)[C@H]2O1 ; O=C1O[C@H]2[C@H](n3ccc(=O)[nH]c3=O)O[C@](F)(COC(=O)c3ccccc3I)[C@H]2O1 ; O=C1O[C@H]2[C@H](n3ccc(=O)[nH]c3=O)O[C@](F)(COC(=O)c3ccc(I)cc3)[C@H]2O1 ; O=C1O[C@H]2[C@H](n3ccc(=O)[nH]c3=O)O[C@](F)(COC(=O)c3ccc(C(F)(F)F)cc3)[C@H]2O1]

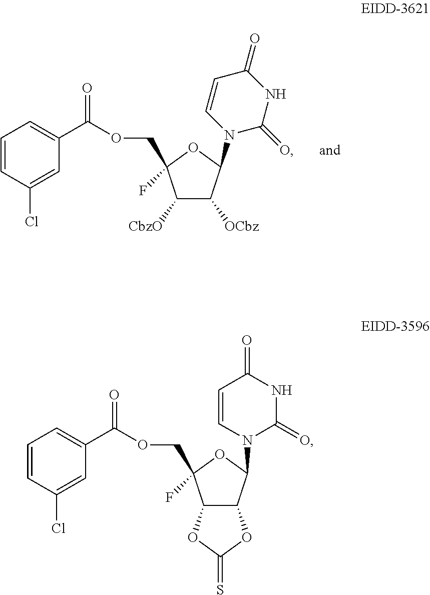

and combinations thereof.
Aspect 97. The compound of any one of claims Aspect 73-Aspect 78, wherein R¹ is a structure represented by a formula:

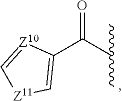

wherein Z¹⁰ is selected from N and C—R^{20j};
wherein Z¹¹ is selected from N—R^{20k} and C—R^{20l}R^{20m};
wherein R^{20j}, R^{20l}, and R^{20m} are each independently selected from hydrogen, deuterium, hydroxyl, amino, cyano, halogen, —OAc, C₁-C₃ alkyl, C₁-C₃ alkoxy, C₁-C₃ alkylamino, (C₁-C₃ alkyl)₂amino, C₁-C₃ carboxamide and lipid;
wherein R^{20j}, R^{20l}, and R^{20m} can each be optionally independently substituted with one or more, the same or different, R¹⁰; and
wherein R^{20k} is selected from hydrogen, deuterium, —OAc, C₁-C₃ alkyl, C₁-C₃ alkoxy, C₁-C₃ alkylamino, (C₁-C₃ alkyl)₂amino, and C₁-C₃ carboxamide.
   Aspect 98. The compound of claim Aspect 97, wherein R¹ is a structure represented by a formula selected from:

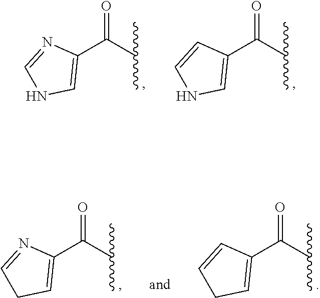

Aspect 99. The compound of any one of claims Aspect 73-Aspect 78, wherein R¹ is a structure represented by a formula selected from:

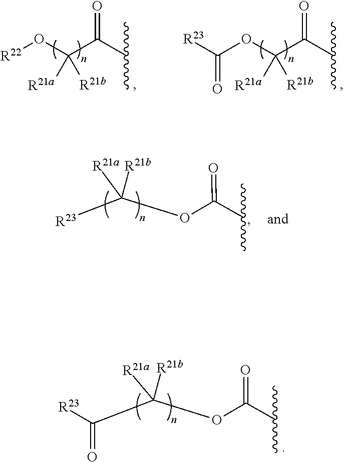

Aspect 100. The compound of any one of claims Aspect 73-Aspect 78, wherein R¹ is a structure represented by a formula selected from:

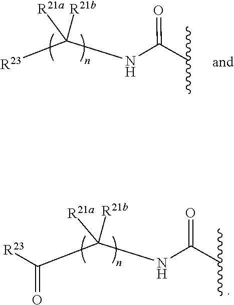

Aspect 101. The compound of any one of claims Aspect 73-Aspect 78, wherein the compound has a structure represented by a formula:

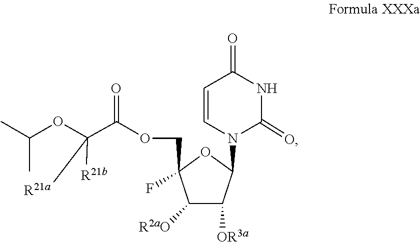

wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can each be optionally covalently bonded, and together with the intermediate atoms, comprise an optionally substituted 5- to 7-membered cycloalkyl or cycloheteroalkyl; and
wherein each R^{21a} and R^{2lb} is independently selected from a (CH₂)_{q}—(C₁-C₁₂ alkoxy).
   Aspect 102. The compound of any one of claims Aspect 73-Aspect 78, wherein the compound has a structure represented by a formula:

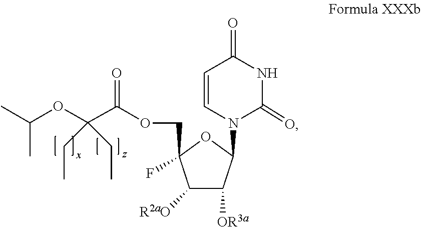

wherein each of x and z is independently selected from 0, 1, 2, 3, 4, 5, 6, 7, 8, 9, and 10; and
wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 5- to 7-membered cycloalkyl or cycloheteroalkyl.
      Aspect 103. The compound of any one of claims Aspect 73-Aspect 78, wherein the compound has a structure represented by a formula:

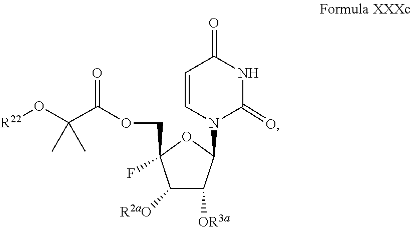

wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 5- to 7-membered cycloalkyl or cycloheteroalkyl;
wherein R²² is selected from hydrogen and C₁-C₂₂ alkyl.
   Aspect 104. The compound of any one of claims Aspect 73-Aspect 103, wherein each of R^{21a}, R^{21b}, and R²³ is independently selected from hydrogen, deuterium, C₁-C₆ alkyl, C₁-C₆ alkylamino, (C₁-C₆ alkyl)₂amino, and C₁-C₆ alkoxy.
   Aspect 105. The compound of claim Aspect 104, wherein each of R^{21a}, R^{21b}, and R²³ is independently selected from hydrogen, deuterium, C₁-C₃ alkyl, C₁-C₃ alkylamino, (C₁-C₃ alkyl)₂amino, and C₁-C₃ alkoxy.
   Aspect 106. The compound of any one of claims Aspect 73-Aspect 105, wherein R²² is selected from hydrogen and C₁-C₂₀ alkyl.
   Aspect 107. The compound of claim Aspect 106, wherein R²² is selected from hydrogen and C₁-C₁₈ alkyl.
   Aspect 108. The compound of claim Aspect 106, wherein R²² is selected from hydrogen and C₁-C₁₆ alkyl.
   Aspect 109. The compound of claim Aspect 106, wherein R²² is selected from hydrogen and C₁-C₁₄ alkyl.
   Aspect 110. The compound of claim Aspect 106, wherein R²² is selected from hydrogen and C₁-C₁₂ alkyl.
   Aspect 111. The compound of claim Aspect 106, wherein R²² is selected from hydrogen and C₁-C₁₀ alkyl.
   Aspect 112. The compound of claim Aspect 106, wherein R²² is selected from hydrogen and C₁-C₈ alkyl.
   Aspect 113. The compound of claim Aspect 106, wherein R²² is selected from hydrogen and C₁-C₆ alkyl.
   Aspect 114. The compound of any one of claims Aspect 73-Aspect 113, wherein R⁴⁰ is selected from hydrogen and C₁-C₃ alkyl.
   Aspect 115. The compound of claim Aspect 114, wherein R⁴⁰ is selected from hydrogen, methyl, and ethyl.
   Aspect 116. The compound of claim Aspect 114, wherein R⁴⁰ is selected from hydrogen and methyl.
   Aspect 117. The compound of claim Aspect 114, wherein R⁴⁰ is hydrogen,
   Aspect 118. The compound of claim Aspect 114, wherein R⁴⁰ is methyl.
   Aspect 119. A compound having a structure represented by a formula:

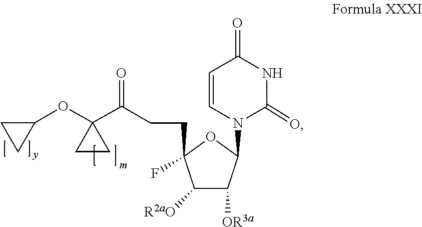

wherein each of m and y is independently selected from 1, 2, 3, and 4;
wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 3- to 7-membered cycloalkyl or cycloheteroalkyl;
      or a pharmaceutically acceptable salt, solvate, or polymorph thereof.
      Aspect 120. The compound of claim Aspect 119, wherein the compound has a structure represented by a formula:

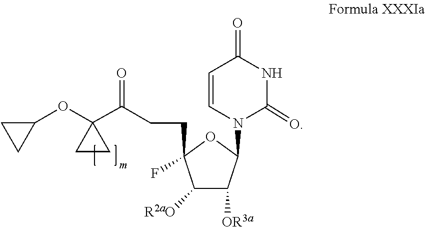

Aspect 121. The compound of claim Aspect 119, wherein the compound has a structure represented by a formula:

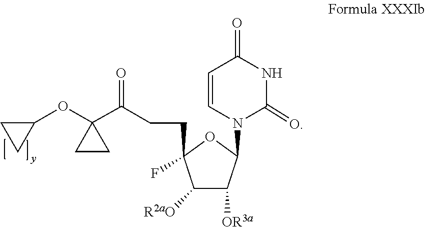

Aspect 122. The compound of claim Aspect 119, wherein the compound is selected from a structure represented by a formula:

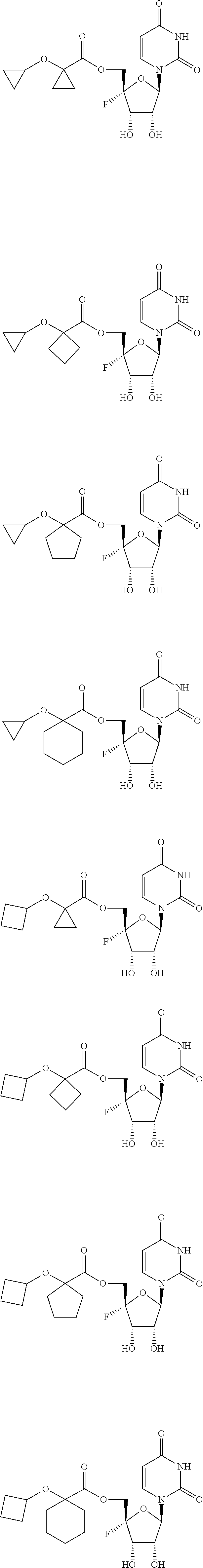

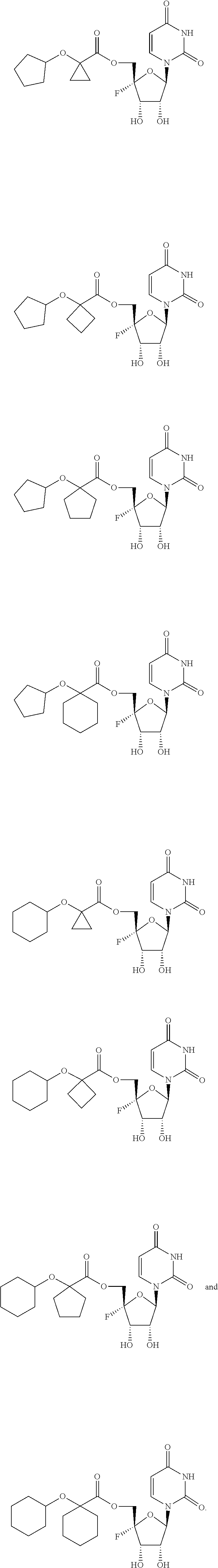

Aspect 122. The compound having a structure by a formula:

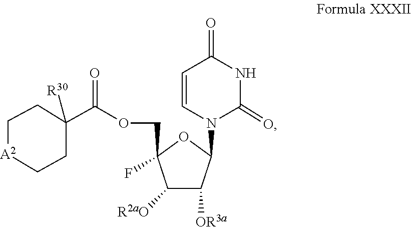

wherein A² is selected from CH₂, 0, S, and NH;
wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 3- to 7-membered cycloalkyl or cycloheteroalkyl; nd
wherein R³⁰ is selected from hydrogen, deuterium, and C₁-C₆ alkyl;
or a pharmaceutically acceptable salt, solvate, or polymorph thereof.
   Aspect 124. The compound of claim Aspect 123, wherein the compound has a structure represented by a formula:

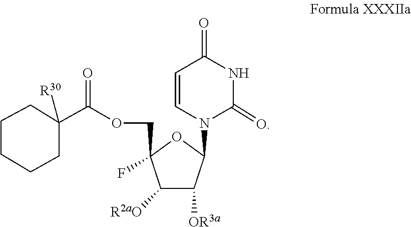

Aspect 125. The compound of claim Aspect 123, wherein the compound has a structure represented by a formula:

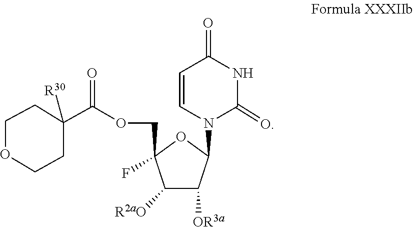

Aspect 126. The compound of claim Aspect 123, wherein the compound has a structure represented by a formula:

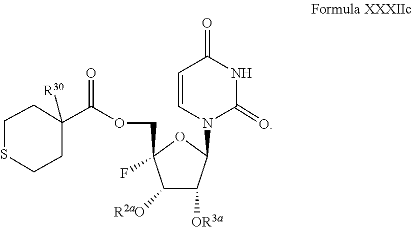

Aspect 127. The compound of claim Aspect 123, wherein the compound has a structure represented by a formula:

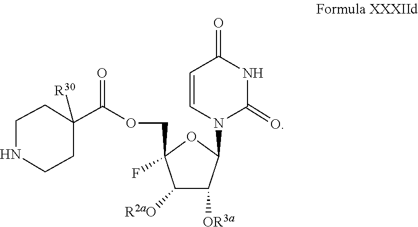

Aspect 128. The compound of any one of claims Aspect 123-Aspect 127, wherein R³⁰ is selected from hydrogen and C₁-C₆ alkyl.
Aspect 129. The compound of claim Aspect 128, wherein R³⁰ is selected from hydrogen, methyl and ethyl.
Aspect 130. The compound of claim Aspect 128, wherein R³⁰ is selected from hydrogen and methyl.
Aspect 131. The compound of claim Aspect 128, wherein R³⁰ is hydrogen.
Aspect 132. The compound of claim Aspect 128, wherein R³⁰ is methyl.
Aspect 133. The compound of claim Aspect 123, wherein the compound is selected from a structure having a formula:

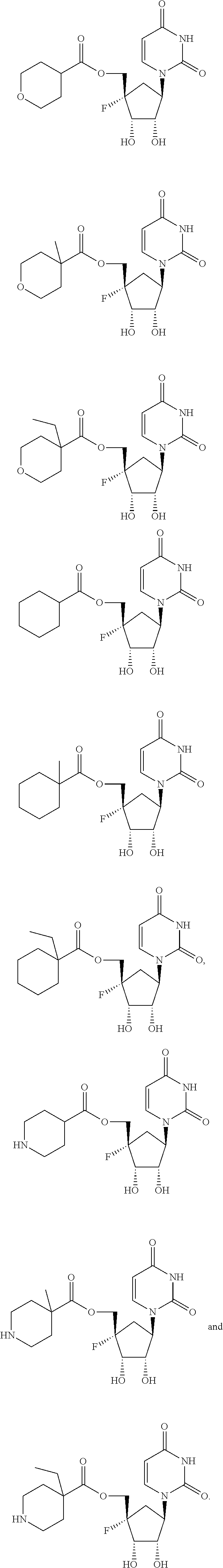

Aspect 134. A compound having a structure represented by a formula:

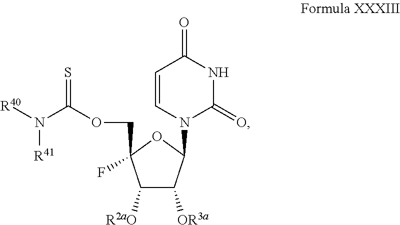

wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 3- to 7-membered cycloalkyl or cycloheteroalkyl;
wherein each of R⁴⁰ and R⁴¹ is independently selected from hydrogen and C₁-C₁₂ alkyl, and wherein each of R⁴⁰ and R⁴¹ can each be optionally independently substituted with one or more, the same or different, R¹⁰;
wherein R¹⁰ is deuterium, hydroxy, azido, thiol, amino, cyano, halogen, alkyl, alkenyl, alkynyl, alkoxy, and alkylamino, wherein R¹⁰ is optionally independently substituted with one or more, the same or different, R¹¹; and
wherein R¹¹ is deuterium, hydroxy, azido, thiol, amino, cyano, halogen, alkyl, alkenyl, alkynyl, alkoxy, alkylamino;
or a pharmaceutically acceptable salt, solvate, or polymorph thereof.
   Aspect 135. A compound having a structure represented by a formula:

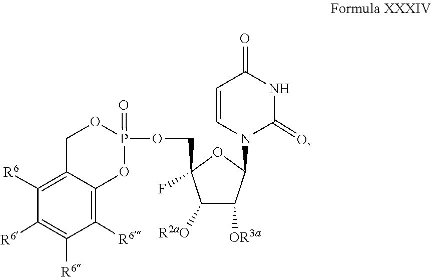

wherein each of R^{2a} and R^{3a} is independently selected from deuterium, hydrogen, —Ac, C₁-C₆ alkyl, C₁-C₆ carboxamide, C₁-C₆ carboxylate, and lipid;
   wherein R^{2a} and R^{3a} can each be optionally independently substituted with one or more, the same or different, R¹⁰; and
   wherein R^{2a} and R^{3a} can be optionally covalently bonded, and together with the intermediate atoms, comprise an optionally substituted 3- to 7-membered cycloalkyl or cycloheteroalkyl;
wherein R⁶, R^{6′}, R^{6″}, and R^{6′″} are each independently selected from hydrogen, deuterium, hydroxyl, amino, cyano, halogen, —OAc, C₁-C₃ alkyl, C₁-C₃ alkoxy, C₁-C₃ alkylamino, (C₁-C₃ alkyl)₂amino, C₁-C₃ carboxamide, and lipid, wherein R⁶, R^{6′}, R^{6″}, and R^{6′″} can each be optionally independently substituted with one or more, the same or different, R¹⁰, provided that at least one of R⁶, R^{6′}, R^{6″}, and R^{6′″} is not hydrogen;
wherein R¹⁰ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, or carbonyl, wherein R¹⁰ is optionally independently substituted with one or more, the same or different, R¹¹;
wherein R¹¹ is deuterium, hydroxy, azido, thiol, amino, cyano, halogen, alkyl, alkenyl, alkynyl, alkoxy, alkylamino, (alkyl)₂amino, allenyl, sulfinyl, sulfamoyl, sulfonyl, lipid, nitro, or carbonyl;
wherein lipid is a C₁-C₂₂ higher alkyl, C₁-C₂₂ higher alkoxy, polyethylene glycol, or aryl substituted with an alkyl group, or a lipid as described herein;
or a pharmaceutically acceptable salt, solvate, or polymorph thereof.
   Aspect 136. The compound of claim Aspect 135, wherein the compound has a structure represented by a formula:

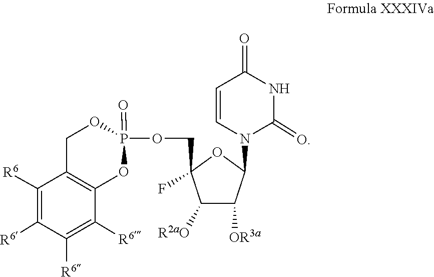

Aspect 137. The compound of claim Aspect 135, wherein the compound has a structure represented by a formula:

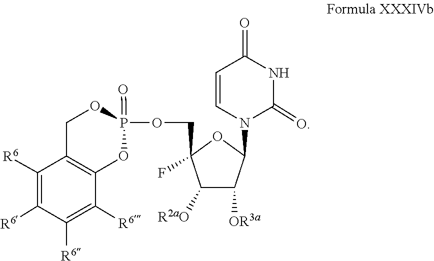

Aspect 138. The compound of any one of claims Aspect 135-Aspect 137, wherein one of R⁶, R^{6′}, R^{6″}, and R^{6′″} is not hydrogen.
Aspect 139. The compound of any one of claims Aspect 135-Aspect 137, wherein two of R⁶, R^{6′}, R^{6″}, and R^{6′″} are not hydrogen.
Aspect 140. The compound of any one of claims Aspect 135-Aspect 137, wherein three of R⁶, R^{6′}, R^{6″}, and R^{6′″} are not hydrogen.
Aspect 141. The compound of any one of claims Aspect 135-Aspect 137, wherein R⁶ is not hydrogen.
Aspect 142. The compound of any one of claims Aspect 135-Aspect 137, wherein R^{6′} is not hydrogen.
Aspect 143. The compound of any one of claims Aspect 135-Aspect 137, wherein R^{6″} is not hydrogen.
Aspect 144. The compound of any one of claims Aspect 135-Aspect 137, wherein R^{6′″} is not hydrogen.
Aspect 145. The compound of any one of claims Aspect 135-Aspect 137, wherein each of R⁶, R^{6′}, and R^{6″} is hydrogen.
Aspect 146. The compound of any one of claims Aspect 135-Aspect 137, wherein each of R^{6′}, R^{6″}, and R^{6′″} is hydrogen.
Aspect 147. The compound of any one of claims Aspect 135-Aspect 137, wherein each of R⁶, R^{6′}, and R^{6′″} is hydrogen.
Aspect 148. The compound of any one of claims Aspect 135-Aspect 137, wherein each of R⁶, R^{6″}, and R^{6′″} is hydrogen.
Aspect 149. The compound of claim Aspect 135, wherein the compound is selected from a structure having a formula:

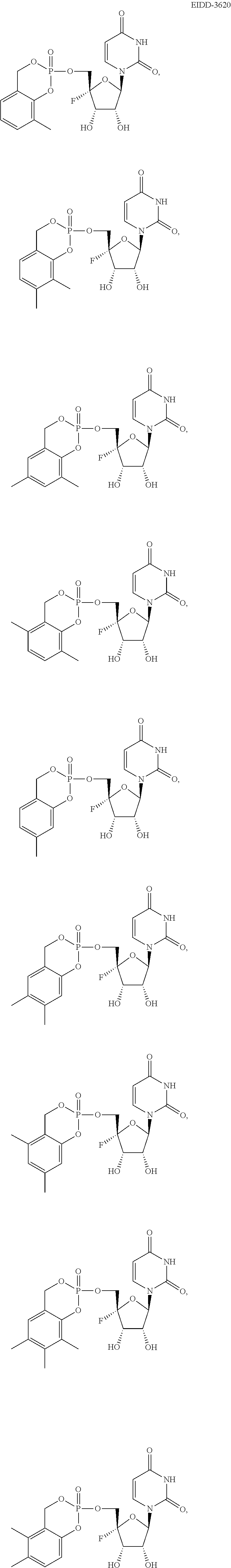

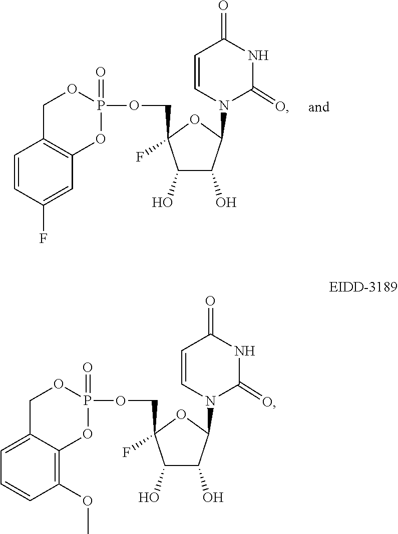 [SMILES: O=c1ccn([C@@H]2O[C@](F)(COP3(=O)OCc4ccc(F)cc4O3)[C@@H](O)[C@H]2O)c(=O)[nH]1 ; "and" ; COc1cccc2c1OP(=O)(OC[C@@]1(F)O[C@@H](n3ccc(=O)[nH]c3=O)[C@H](O)[C@@H]1O)OC2]

and combinations of the foregoing.
Aspect 150. A compound selected from the following:

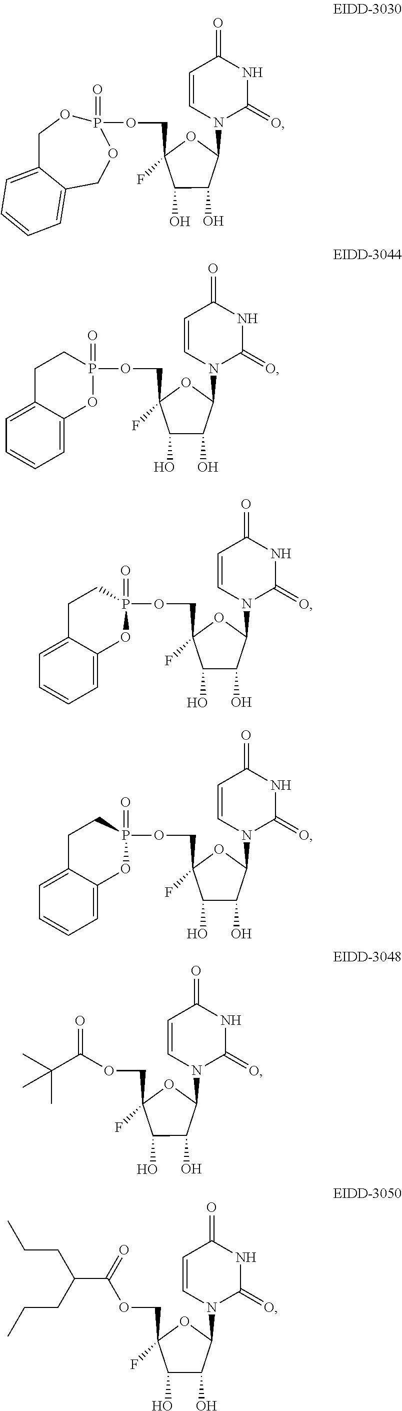

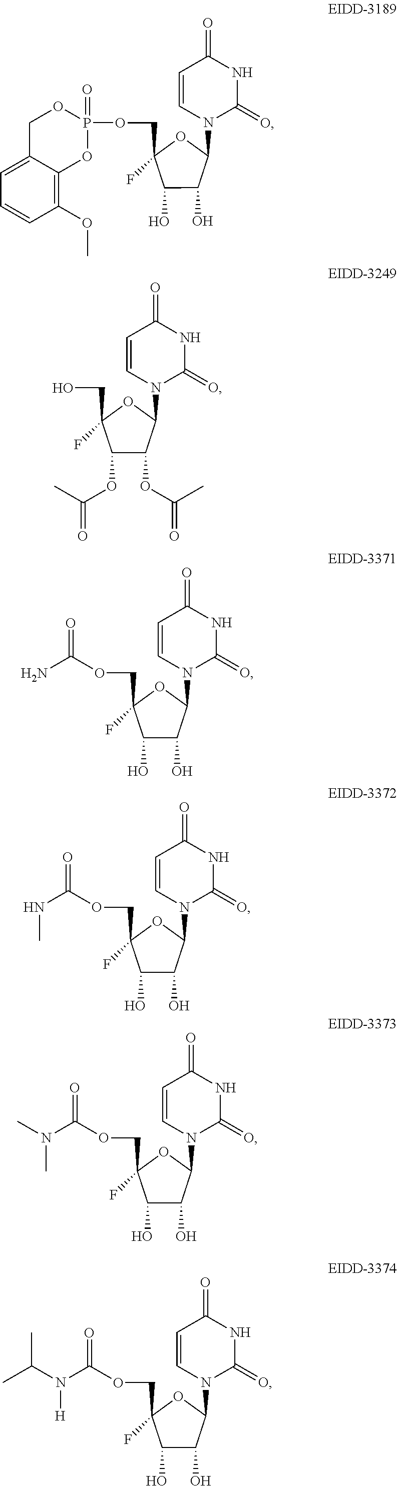

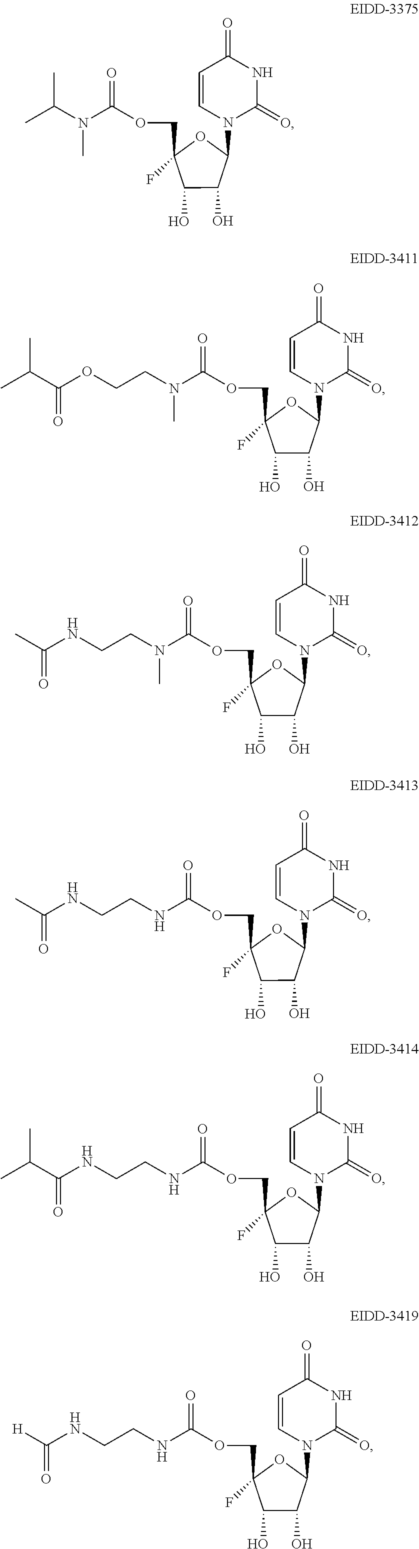 [SMILES: CC(C)N(C)C(=O)OC[C@@]1(F)O[C@@H](n2ccc(=O)[nH]c2=O)[C@H](O)[C@@H]1O ; CC(C)C(=O)OCCN(C)C(=O)OC[C@@]1(F)O[C@@H](n2ccc(=O)[nH]c2=O)[C@H](O)[C@@H]1O ; CC(=O)NCCN(C)C(=O)OC[C@@]1(F)O[C@@H](n2ccc(=O)[nH]c2=O)[C@H](O)[C@@H]1O ; CC(=O)NCCNC(=O)OC[C@@]1(F)O[C@@H](n2ccc(=O)[nH]c2=O)[C@H](O)[C@@H]1O ; CC(C)C(=O)NCCNC(=O)OC[C@@]1(F)O[C@@H](n2ccc(=O)[nH]c2=O)[C@H](O)[C@@H]1O ; O=CNCCNC(=O)OC[C@@]1(F)O[C@@H](n2ccc(=O)[nH]c2=O)[C@H](O)[C@@H]1O]

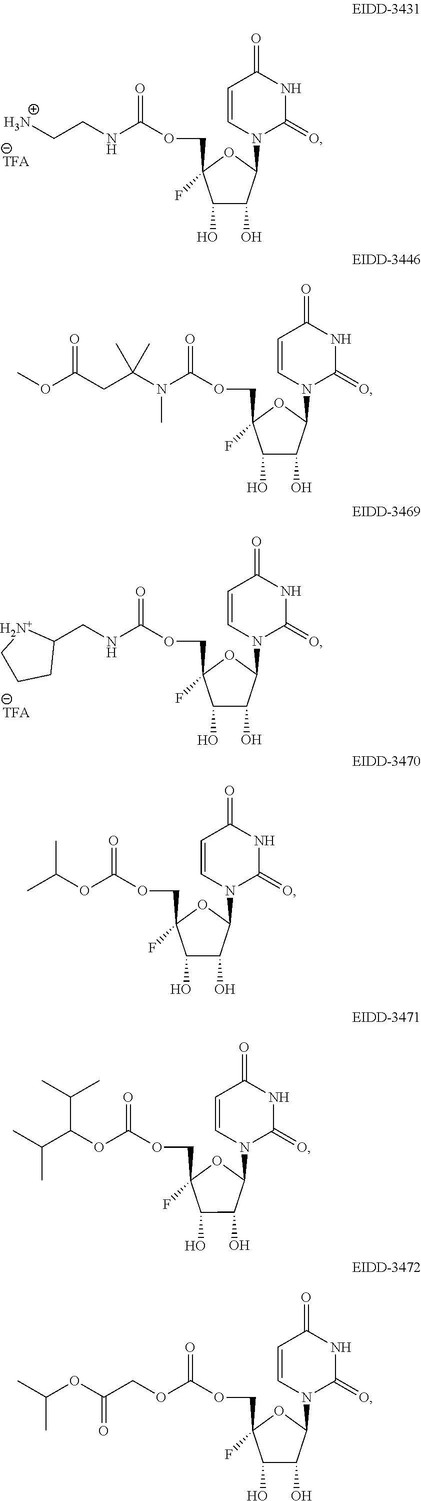 [SMILES: [NH3+]CCNC(=O)OC[C@@]1(F)O[C@@H](n2ccc(=O)[nH]c2=O)[C@H](O)[C@@H]1O ; COC(=O)CC(C)(C)N(C)C(=O)OC[C@@]1(F)O[C@@H](n2ccc(=O)[nH]c2=O)[C@H](O)[C@@H]1O ; O=C(NCC1CCC[NH2+]1)OC[C@@]1(F)O[C@@H](n2ccc(=O)[nH]c2=O)[C@H](O)[C@@H]1O ; CC(C)OC(=O)OC[C@@]1(F)O[C@@H](n2ccc(=O)[nH]c2=O)[C@H](O)[C@@H]1O ; CC(C)C(OC(=O)OC[C@@]1(F)O[C@@H](n2ccc(=O)[nH]c2=O)[C@H](O)[C@@H]1O)C(C)C ; CC(C)OC(=O)COC(=O)OC[C@@]1(F)O[C@@H](n2ccc(=O)[nH]c2=O)[C@H](O)[C@@H]1O]

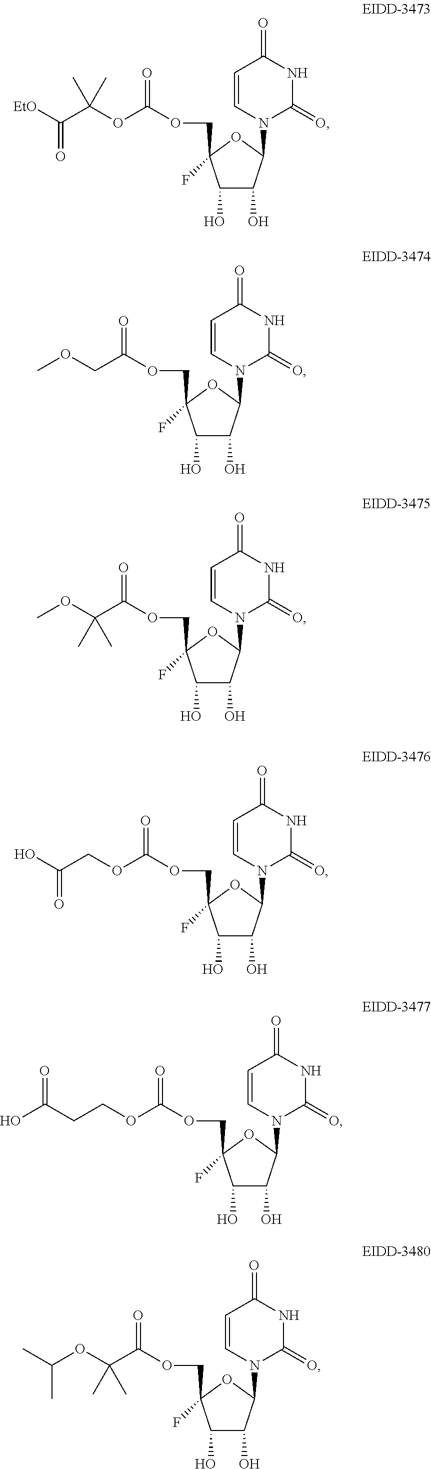 [SMILES: CCOC(=O)C(C)(C)OC(=O)OC[C@@]1(F)O[C@@H](n2ccc(=O)[nH]c2=O)[C@H](O)[C@@H]1O ; COCC(=O)OC[C@@]1(F)O[C@@H](n2ccc(=O)[nH]c2=O)[C@H](O)[C@@H]1O ; COC(C)(C)C(=O)OC[C@@]1(F)O[C@@H](n2ccc(=O)[nH]c2=O)[C@H](O)[C@@H]1O ; O=C(O)COC(=O)OC[C@@]1(F)O[C@@H](n2ccc(=O)[nH]c2=O)[C@H](O)[C@@H]1O ; O=C(O)CCOC(=O)OC[C@@]1(F)O[C@@H](n2ccc(=O)[nH]c2=O)[C@H](O)[C@@H]1O ; CC(C)OC(C)(C)C(=O)OC[C@@]1(F)O[C@@H](n2ccc(=O)[nH]c2=O)[C@H](O)[C@@H]1O]

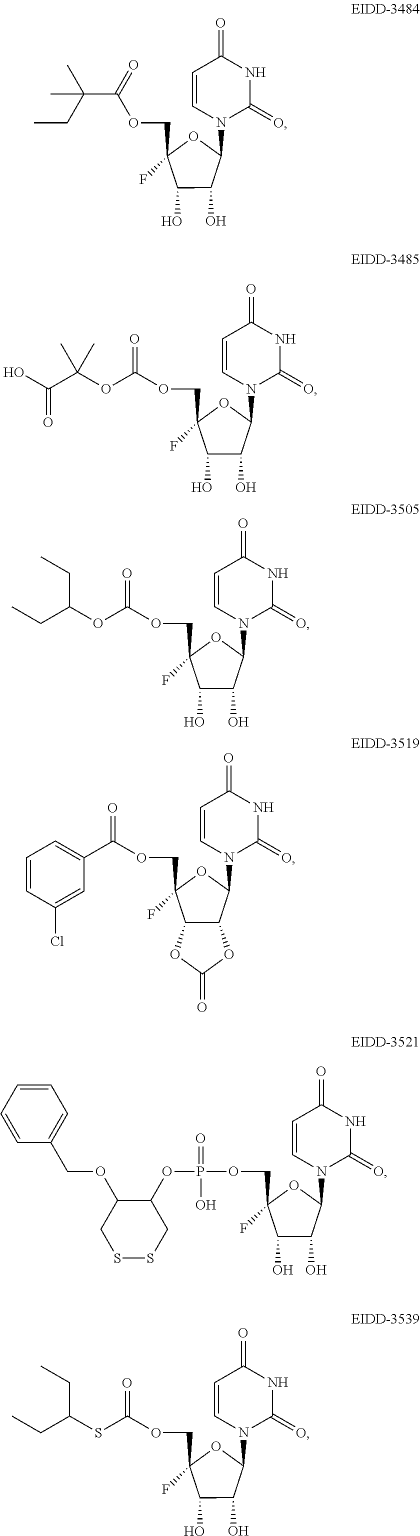

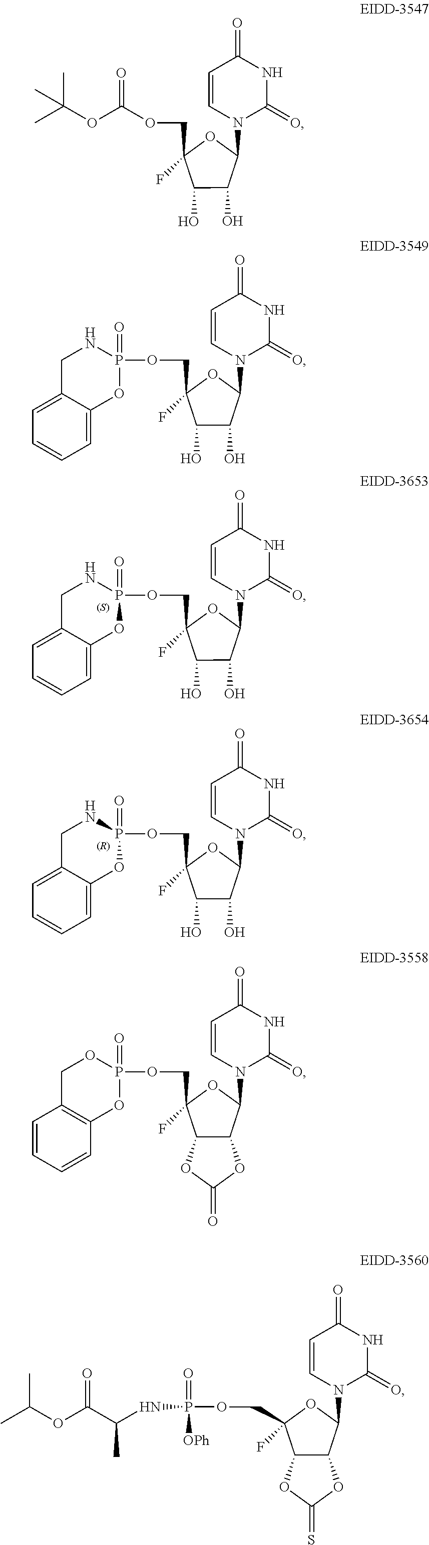

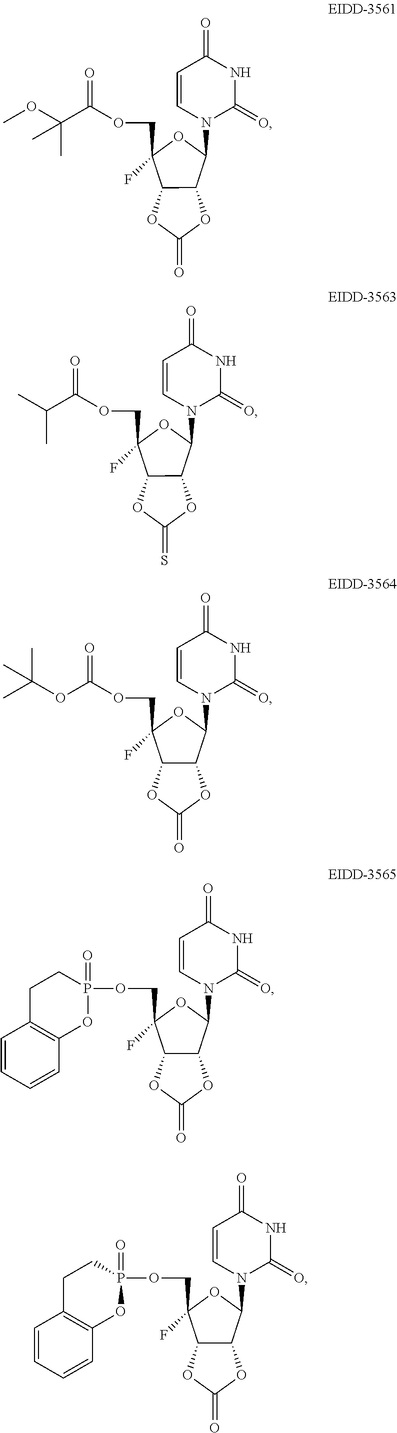

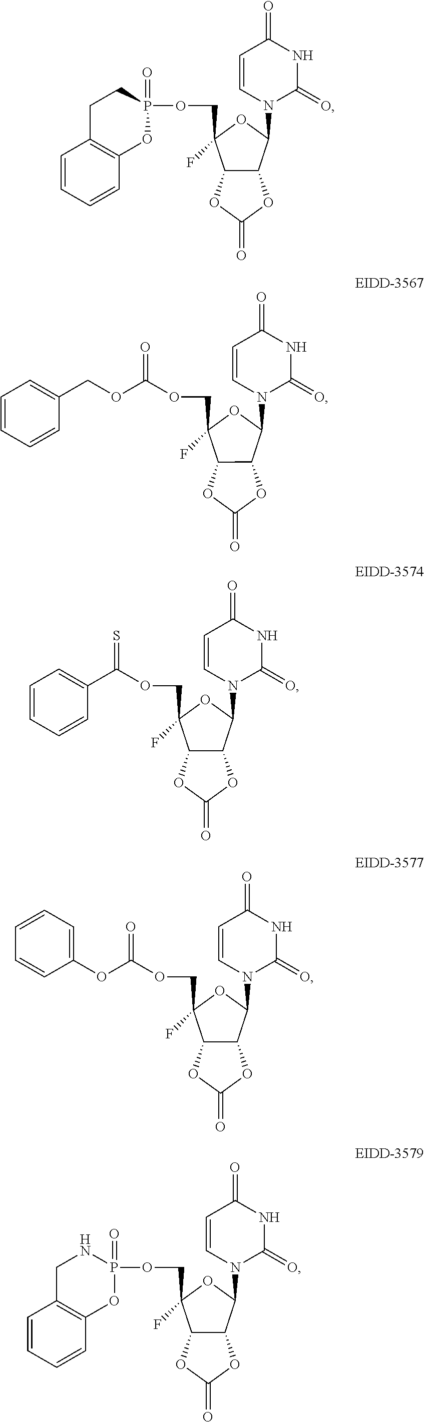

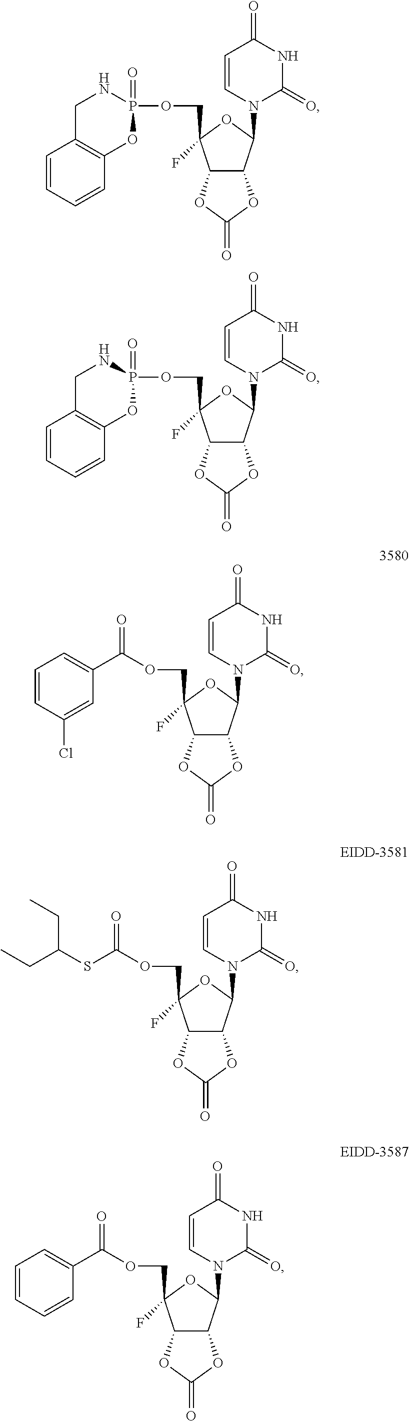

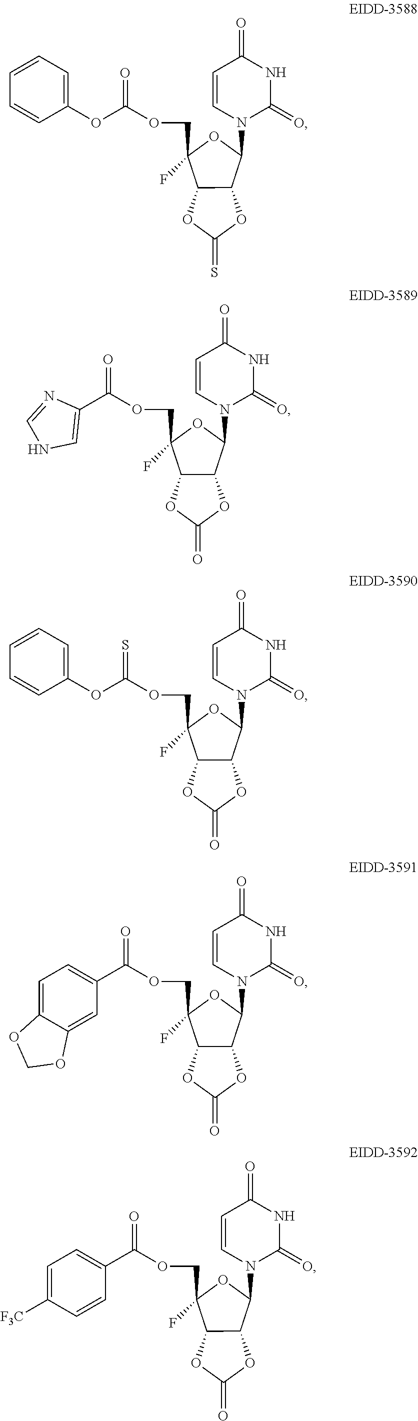

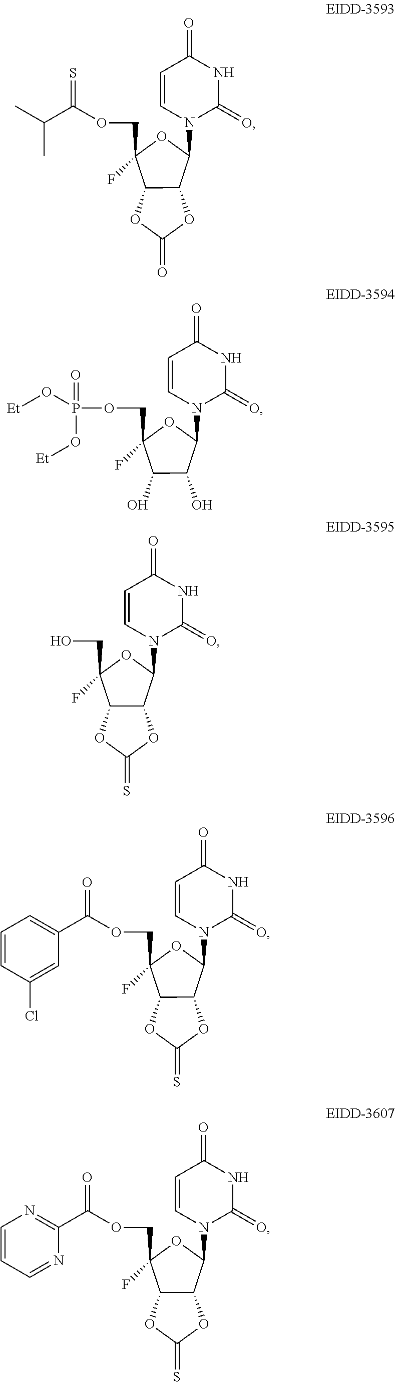

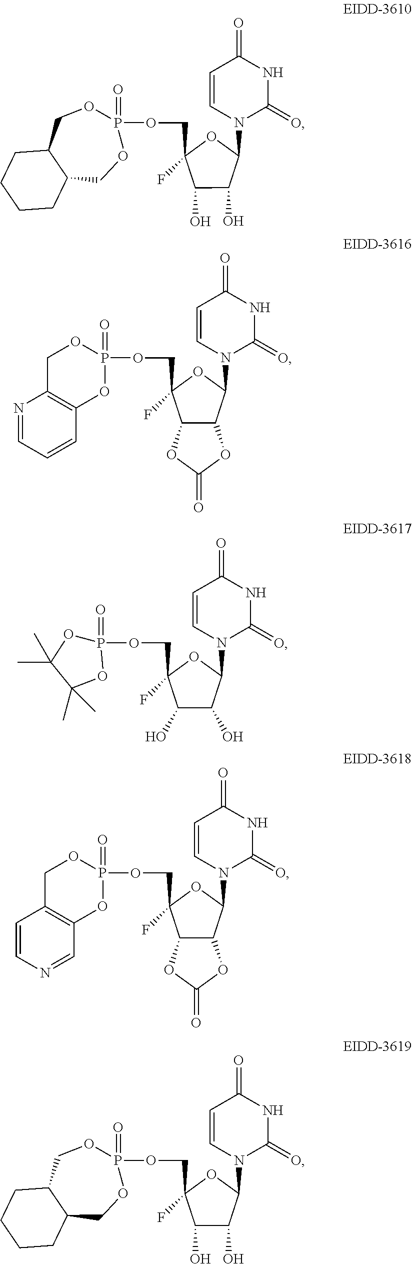

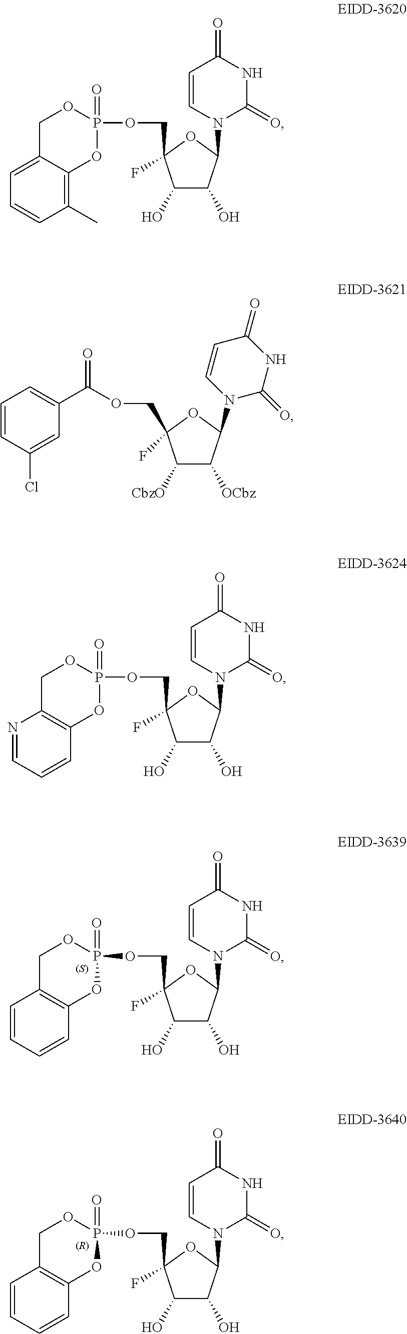

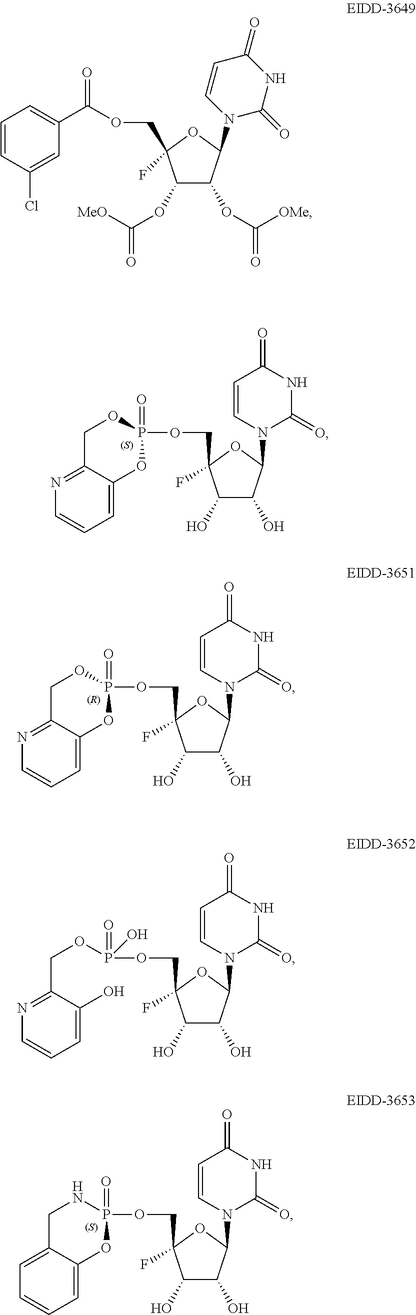

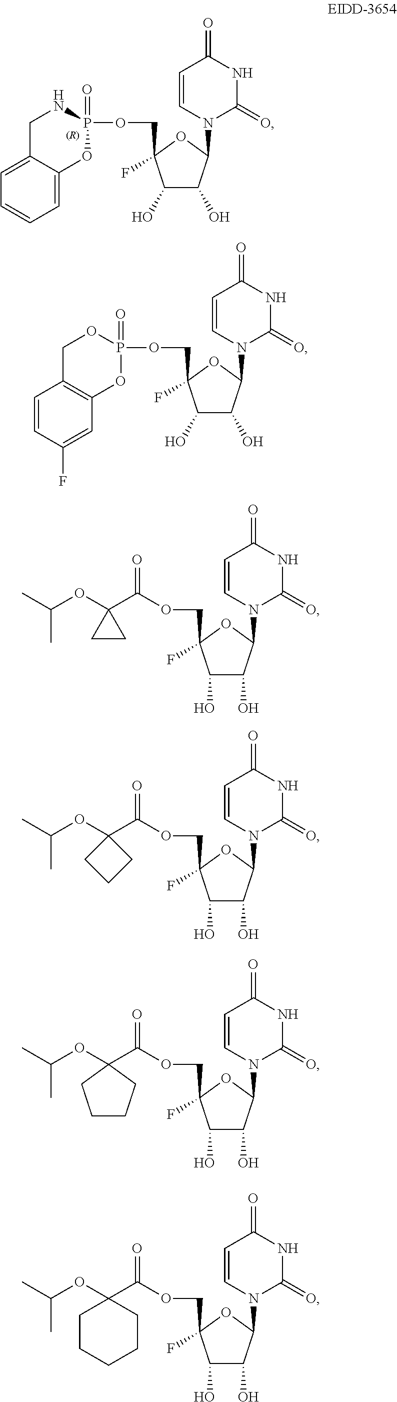

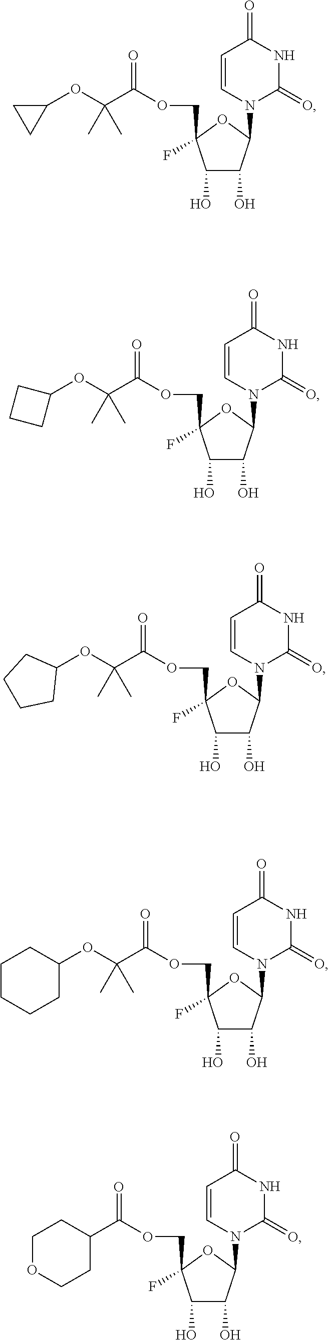 [SMILES: CC(C)(OC1CC1)C(=O)OC[C@@]1(F)O[C@@H](n2ccc(=O)[nH]c2=O)[C@H](O)[C@@H]1O ; CC(C)(OC1CCC1)C(=O)OC[C@@]1(F)O[C@@H](n2ccc(=O)[nH]c2=O)[C@H](O)[C@@H]1O ; CC(C)(OC1CCCC1)C(=O)OC[C@@]1(F)O[C@@H](n2ccc(=O)[nH]c2=O)[C@H](O)[C@@H]1O ; CC(C)(OC1CCCCC1)C(=O)OC[C@@]1(F)O[C@@H](n2ccc(=O)[nH]c2=O)[C@H](O)[C@@H]1O ; O=C(OC[C@@]1(F)O[C@@H](n2ccc(=O)[nH]c2=O)[C@H](O)[C@@H]1O)C1CCOCC1]

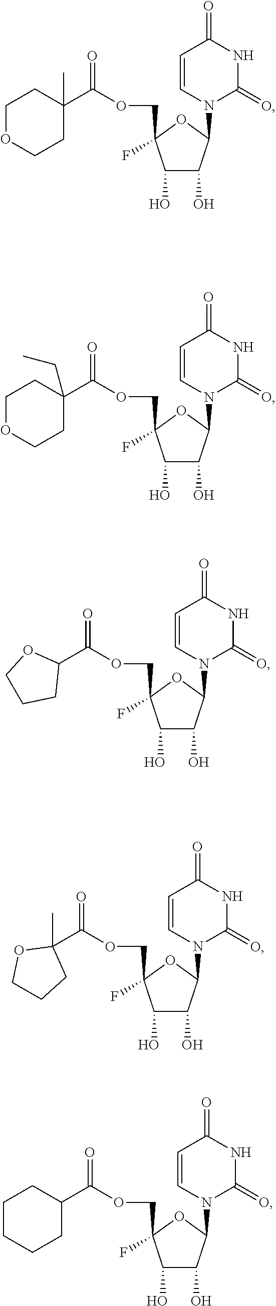 [SMILES: CC1(C(=O)OC[C@@]2(F)O[C@@H](n3ccc(=O)[nH]c3=O)[C@H](O)[C@@H]2O)CCOCC1 ; CCC1(C(=O)OC[C@@]2(F)O[C@@H](n3ccc(=O)[nH]c3=O)[C@H](O)[C@@H]2O)CCOCC1 ; O=C(OC[C@@]1(F)O[C@@H](n2ccc(=O)[nH]c2=O)[C@H](O)[C@@H]1O)C1CCCO1 ; CC1(C(=O)OC[C@@]2(F)O[C@@H](n3ccc(=O)[nH]c3=O)[C@H](O)[C@@H]2O)CCCO1 ; O=C(OC[C@@]1(F)O[C@@H](n2ccc(=O)[nH]c2=O)[C@H](O)[C@@H]1O)C1CCCCC1]

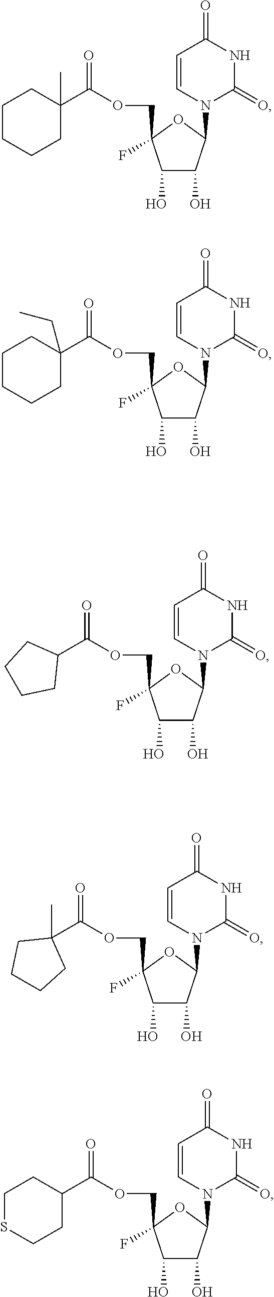 [SMILES: CC1(C(=O)OC[C@@]2(F)O[C@@H](n3ccc(=O)[nH]c3=O)[C@H](O)[C@@H]2O)CCCCC1 ; CCC1(C(=O)OC[C@@]2(F)O[C@@H](n3ccc(=O)[nH]c3=O)[C@H](O)[C@@H]2O)CCCCC1 ; O=C(OC[C@@]1(F)O[C@@H](n2ccc(=O)[nH]c2=O)[C@H](O)[C@@H]1O)C1CCCC1 ; CC1(C(=O)OC[C@@]2(F)O[C@@H](n3ccc(=O)[nH]c3=O)[C@H](O)[C@@H]2O)CCCC1 ; O=C(OC[C@@]1(F)O[C@@H](n2ccc(=O)[nH]c2=O)[C@H](O)[C@@H]1O)C1CCSCC1]

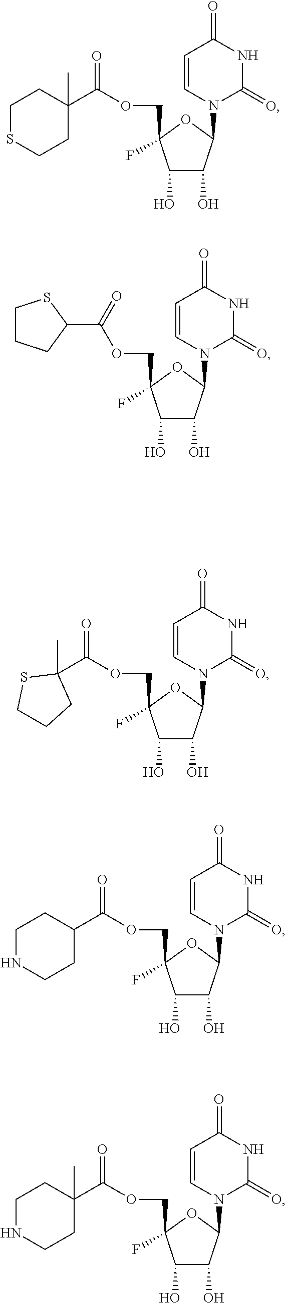 [SMILES: CC1(C(=O)OC[C@@]2(F)O[C@@H](n3ccc(=O)[nH]c3=O)[C@H](O)[C@@H]2O)CCSCC1 ; O=C(OC[C@@]1(F)O[C@@H](n2ccc(=O)[nH]c2=O)[C@H](O)[C@@H]1O)C1CCCS1 ; CC1(C(=O)OC[C@@]2(F)O[C@@H](n3ccc(=O)[nH]c3=O)[C@H](O)[C@@H]2O)CCCS1 ; O=C(OC[C@@]1(F)O[C@@H](n2ccc(=O)[nH]c2=O)[C@H](O)[C@@H]1O)C1CCNCC1 ; CC1(C(=O)OC[C@@]2(F)O[C@@H](n3ccc(=O)[nH]c3=O)[C@H](O)[C@@H]2O)CCNCC1]

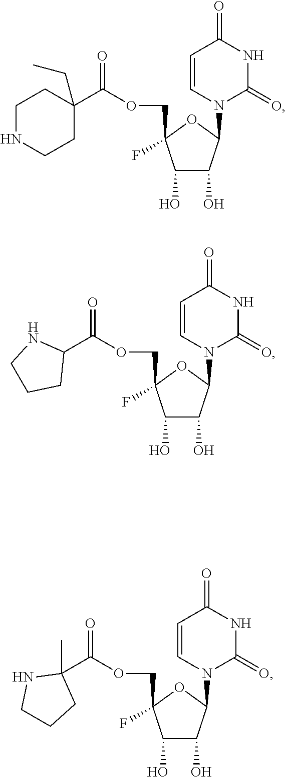 [SMILES: CCC1(C(=O)OC[C@@]2(F)O[C@@H](n3ccc(=O)[nH]c3=O)[C@H](O)[C@@H]2O)CCNCC1 ; O=C(OC[C@@]1(F)O[C@@H](n2ccc(=O)[nH]c2=O)[C@H](O)[C@@H]1O)C1CCCN1 ; CC1(C(=O)OC[C@@]2(F)O[C@@H](n3ccc(=O)[nH]c3=O)[C@H](O)[C@@H]2O)CCCN1]

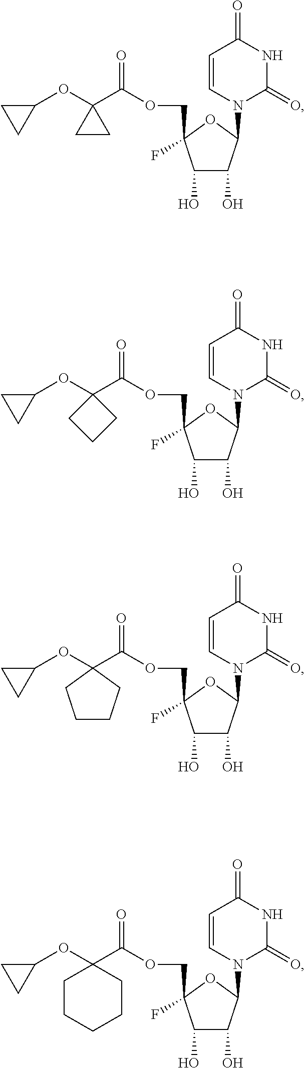 [SMILES: O=C(OC[C@@]1(F)O[C@@H](n2ccc(=O)[nH]c2=O)[C@H](O)[C@@H]1O)C1(OC2CC2)CC1 ; O=C(OC[C@@]1(F)O[C@@H](n2ccc(=O)[nH]c2=O)[C@H](O)[C@@H]1O)C1(OC2CC2)CCC1 ; O=C(OC[C@@]1(F)O[C@@H](n2ccc(=O)[nH]c2=O)[C@H](O)[C@@H]1O)C1(OC2CC2)CCCC1 ; O=C(OC[C@@]1(F)O[C@@H](n2ccc(=O)[nH]c2=O)[C@H](O)[C@@H]1O)C1(OC2CC2)CCCCC1]

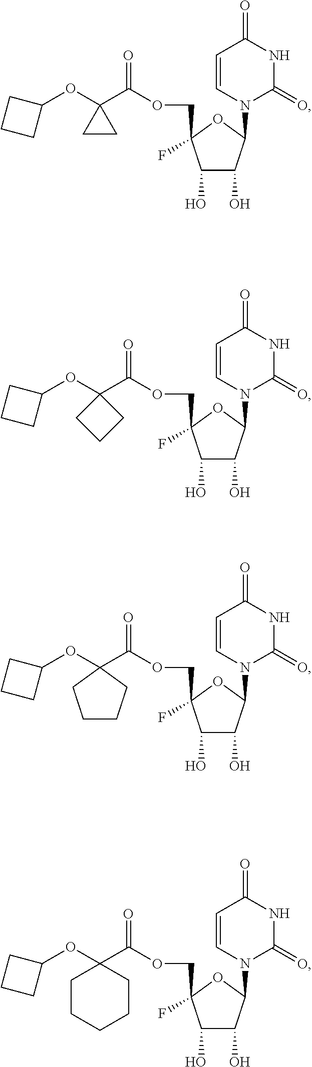 [SMILES: O=C(OC[C@@]1(F)O[C@@H](n2ccc(=O)[nH]c2=O)[C@H](O)[C@@H]1O)C1(OC2CCC2)CC1 ; O=C(OC[C@@]1(F)O[C@@H](n2ccc(=O)[nH]c2=O)[C@H](O)[C@@H]1O)C1(OC2CCC2)CCC1 ; O=C(OC[C@@]1(F)O[C@@H](n2ccc(=O)[nH]c2=O)[C@H](O)[C@@H]1O)C1(OC2CCC2)CCCC1 ; O=C(OC[C@@]1(F)O[C@@H](n2ccc(=O)[nH]c2=O)[C@H](O)[C@@H]1O)C1(OC2CCC2)CCCCC1]

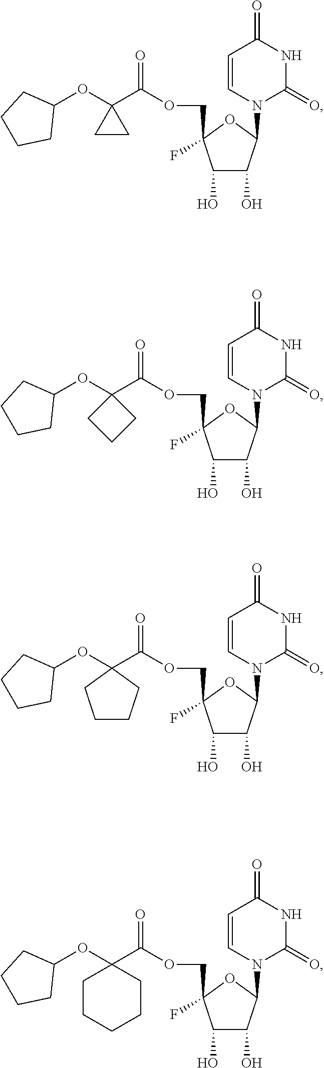 [SMILES: O=C(OC[C@@]1(F)O[C@@H](n2ccc(=O)[nH]c2=O)[C@H](O)[C@@H]1O)C1(OC2CCCC2)CC1 ; O=C(OC[C@@]1(F)O[C@@H](n2ccc(=O)[nH]c2=O)[C@H](O)[C@@H]1O)C1(OC2CCCC2)CCC1 ; O=C(OC[C@@]1(F)O[C@@H](n2ccc(=O)[nH]c2=O)[C@H](O)[C@@H]1O)C1(OC2CCCC2)CCCC1 ; O=C(OC[C@@]1(F)O[C@@H](n2ccc(=O)[nH]c2=O)[C@H](O)[C@@H]1O)C1(OC2CCCC2)CCCCC1]

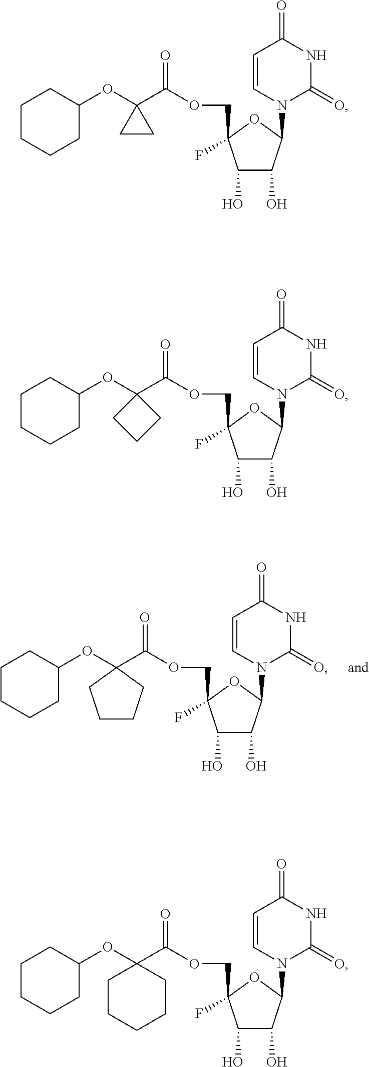 [SMILES: O=C(OC[C@@]1(F)O[C@@H](n2ccc(=O)[nH]c2=O)[C@H](O)[C@@H]1O)C1(OC2CCCCC2)CC1 ; O=C(OC[C@@]1(F)O[C@@H](n2ccc(=O)[nH]c2=O)[C@H](O)[C@@H]1O)C1(OC2CCCCC2)CCC1 ; O=C(OC[C@@]1(F)O[C@@H](n2ccc(=O)[nH]c2=O)[C@H](O)[C@@H]1O)C1(OC2CCCCC2)CCCC1 ; O=C(OC[C@@]1(F)O[C@@H](n2ccc(=O)[nH]c2=O)[C@H](O)[C@@H]1O)C1(OC2CCCCC2)CCCCC1]

or a pharmaceutically acceptable salt, solvate, or polymorph thereof.
Aspect 151. A pharmaceutical composition comprising a pharmaceutically acceptable excipient and a compound selected from one or more compound of claims Aspect 73-Aspect 150, or a pharmaceutical or physiological salt thereof.
Aspect 152. The pharmaceutical composition of claim Aspect 151, further comprising a propellant.
Aspect 153. The pharmaceutical composition of claim Aspect 152, wherein the propellant is compressed air, ethanol, nitrogen, carbon dioxide, nitrous oxide, hydrofluoroalkanes (HFA), 1,1,1,2,-tetrafluoroethane, 1,1,1,2,3,3,3-heptafluoropropane or combinations thereof.
Aspect 154. A pressurized container comprising a pharmaceutical composition of claim Aspect 151.
Aspect 155. The container of claim Aspect 154, wherein the container is a manual pump spray, inhaler, meter-dosed inhaler, dry powder inhaler, nebulizer, vibrating mesh nebulizer, jet nebulizer, or ultrasonic wave nebulizer.
Aspect 156. A method of treating or preventing a viral infection comprising administering in effective amount of the compound of any one of claims Aspect 73-Aspect 150, or a pharmaceutical or physiological salt thereof, or the pharmaceutical composition of any one of claims Aspect 151-Aspect 153 to a subject in need thereof.
Aspect 157. The method of claim Aspect 156, wherein the viral infection is a Togaviridae infection.
Aspect 158. The method of claim Aspect 157, wherein the Togaviridae infection is an infection with a virus selected from Eastern equine encephalitis virus, Western equine encephalitis virus, Venezuelan equine encephalitis virus, Chikungunya virus, and Ross River virus.
Aspect 159. The method of claim Aspect 156, wherein the viral infection is a Coronaviridae.
Aspect 160. The method of claim Aspect 159, wherein the viral infection is a human coronavirus infection, SARS coronavirus infection, or MERS coronavirus infection.
Aspect 161. The method of claim Aspect 159 or claim Aspect 160, wherein the SARS coronavirus infection is an infection with a SARS-CoV2 virus.
Aspect 162. The method of claim Aspect 161, wherein the SARS-CoV2 virus comprises variants of SARS-CoV-2, including, but are not limited to, the more virulent strain originating in Brazil, known as P.1; the variant originating in the United Kingdom, known as 20I/501Y.V1, VOC 202012/01, or B.1.1.7; and the variant originating in South Africa, known as 20H/501Y.V2 or B.1.351; as well as further variants and lineages that derive therefrom.
Aspect 163. The method of claim Aspect 156, wherein the viral infection is an Orthomyxoviridae virus.
Aspect 164. The method of claim Aspect 163, wherein the viral infection is influenza A virus and influenza B virus.
Aspect 165. The method of claim Aspect 156, wherein the viral infection is a Pneumoviridae.
Aspect 166. The method of claim Aspect 165, wherein the viral infection is RSV.
Aspect 167. The method of claim Aspect 156, wherein the viral infection is an Arenaviridae.
Aspect 168. The method of claim Aspect 167, wherein the viral infection is Tacaribe virus, Pichinde virus, Junin virus, Lassa fever virus, and Lymphocytic Choriomeningitis virus.
Aspect 169. The method of claim Aspect 156, wherein the viral infection is Bunyaviridae.
Aspect 170. The method of claim Aspect 169, wherein the viral infection is Rift Valley fever virus, Punta Toro virus, LaCrosse virus, Maporal virus, Heartland virus, and Severe Fever Thrombocytopenia Syndrome virus.
Aspect 171. The method of claim Aspect 156, wherein the viral infection is Flaviviridae.
Aspect 172. The method of claim Aspect 171, wherein the viral infection is Zika virus, Dengue virus 1, Dengue virus 2, Dengue virus 3, Dengue virus 4, West Nile virus, Yellow fever virus, Japanese encephalitis virus, Powassen virus, Usutu virus, and tick-borne encephalitis virus.
Aspect 173. The method of claim Aspect 156, wherein the viral infection is Picornaviridae.
Aspect 174. The method of claim Aspect 173, wherein the viral infection is poliovirus, Coxsackie virus, enterovirus.
Aspect 175. The method of claim Aspect 156, wherein the viral infection is comprises an infection with a human coronavirus, SARS coronavirus, MERS coronavirus, Eastern equine encephalitis virus, Western equine encephalitis virus, Venezuelan equine encephalitis virus, Chikungunya virus, Ross River virus, RSV, influenza A virus, influenza B virus, Tacaribe virus, Pichinde virus, Junin virus, Lassa fever virus, Lymphocytic Choriomeningitis virus, Rift Valley fever virus, Punta Toro virus, LaCrosse virus, Maporal virus, Heartland virus, and Severe Fever Thrombocytopenia Syndrome virus, poliovirus, norovirus, enterovirus, a coxsackie virus A, B and C, coxsackie A16, EV-D68, EV-A71, rhinovirus, poliovirus, echovirus, picornaviruses, cardioviruses, enteroviruses, erboviruses, hepatovirus, kobuviruses, parechoviruses, teschoviruses, caliciviruses, which include noroviruses, sapoviruses, lagoviruses, vesiviruses, astroviruses, togaviruses, flaviviruses, hepacivirus, coronaviruses, arteriviruses, rhabdoviruses, paramyxoviruses, orthomyxoviruses, hantaviruses, reoviruses, rotaviruses, birnaviruses, chrysoviruses, cystoviruses, hypoviruses partitiviruses, totoviruses, lentiviruses, polyomaviruses, papillomaviruses, adenoviruses, circoviruses, parvoviruses, erythroviruses, betaparvoviruses, amdoviruses, densoviruses, iteraviruses, brevidensoviruses, pefudensoviruses, herpes viruses 1, 2, 3, 4, 5, 6, 7 and 8, poxviruses, hepadnaviruses, pneumovirus, bunyavirus, arenavirus, or orthomyxovirus.
Aspect 176. The method of any one of claims Aspect 156-Aspect 175, wherein the method further comprises administering a second antiviral agent.
Aspect 177. The method of claim Aspect 176, wherein the second antiviral agent is selected from remdesivir, favipiravir, darunavir, nelfinavir, saquinavir, lopinavir, ritonavir, remdesivir, paxlovid, molnupiravir, ABX464, favilavir, niclosamide, laninamivir, oseltamivir, zanamivir, peramivir, CS-8958, ribavirin, amantadine, rimantadine, tamiphosphor guanidine monoester, or phosphazanamivir or its monoester, disoxaril, pleconaril, pirodavir, vapendavir, pocapavir, azaglutamine, S-nitroso-N-acetyl-penicillamine (SNAP), glyceryl trinitrate (GTN), isosorbide dinitrate (ISDN), glycerrhizin, 5-(3,4-dichlorophenyl) methylhydantoin, AG7088, pleconaril, 3-methylthio-5-aryl-4-isothiazolecarbonitrile, a pyridyl imidazolidinone, ribavirin, mycophenolic acid, 6-azauridine, pyrazofurin, 3-methylkaempferol, and derivatives, physiological or pharmaceutical salts, or prodrugs thereof, and combinations thereof.
Aspect 178. The method of claim Aspect 177, wherein the second antiviral agent is selected from remdesivir, favipiravir, darunavir, nelfinavir, saquinavir, lopinavir, ritonavir, remdesivir, paxlovid, molnupiravir, ABX464, favilavir, and niclosamide, and derivatives, physiological or pharmaceutical salts, or prodrugs thereof, and combinations thereof.
Aspect 179. The method of claim Aspect 177, wherein the second antiviral agent is selected from laninamivir, oseltamivir, zanamivir, peramivir, CS-8958, ribavirin, amantadine, rimantadine, tamiphosphor guanidine monoester, and phosphazanamivir or its monoester, and derivatives, physiological or pharmaceutical salts, or prodrugs thereof, and combinations thereof.
Aspect 180. A method of treating or preventing a viral infection comprising administering to a subject in need thereof an effective amount of the compound of any one of claims Aspect 73-Aspect 150, or a pharmaceutical or physiological salt thereof, or the pharmaceutical composition of any one of claims Aspect 151-Aspect 153;
wherein the subject is administered a loading dose of the compound or the pharmaceutical composition in a first treatment period; and
wherein the subject is administered a treatment dose of the pharmaceutical composition in a second treatment period following the first treatment period.
   Aspect 181. The method of claim Aspect 180, wherein the first treatment period is days 1-5 following diagnosis of the viral infection or presentation for preventing the viral infection.
   Aspect 182. The method of claim Aspect 181, wherein the first treatment period is days 1-2 following diagnosis of the viral infection or presentation for preventing the viral infection.
   Aspect 183. The method of claim Aspect 181, wherein the first treatment period is day 1 following diagnosis of the viral infection or presentation for preventing the viral infection.
   Aspect 184. The method of any one of claims Aspect 180-Aspect 183, wherein the loading dose is about 1.1-fold to about 10-fold the treatment dose.
   Aspect 185. The method of claim Aspect 184, wherein the loading dose is about 1.5-fold to about 5-fold the treatment dose.
   Aspect 186. The method of claim Aspect 184, wherein the loading dose is about 1.5-fold to about 2.5-fold the treatment dose.
   Aspect 187. The method of any one of claims Aspect 180-Aspect 186, wherein the loading dose is administered once daily, two times daily, three times daily, or four times daily.
   Aspect 188. The method of any one of claims Aspect 180-Aspect 186, wherein the loading dose is administered at least twice daily.
   Aspect 189. The method of claim Aspect 187 or claim Aspect 188, wherein the loading dose divided equally among the number of times administered daily.
   Aspect 190. A method of treating or preventing a viral infection comprising administering to a subject in need thereof an effective amount of a compound having a structure represented by a formula:

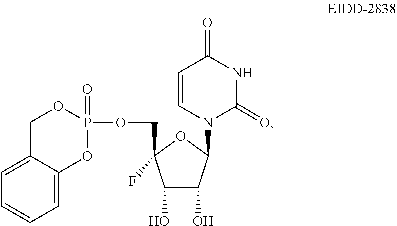 [SMILES: O=c1ccn([C@@H]2O[C@](F)(COP3(=O)OCc4ccccc4O3)[C@@H](O)[C@H]2O)c(=O)[nH]1]

or a pharmaceutical or physiological salt thereof, or a pharmaceutical composition of the foregoing compound;
wherein the subject is administered a loading dose of the compound or the pharmaceutical composition in a first treatment period; and
wherein the subject is administered a treatment dose of the pharmaceutical composition in a second treatment period following the first treatment period.
   Aspect 191. The method of claim Aspect 190, wherein the first treatment period is days 1-5 following diagnosis of the viral infection or presentation for preventing the viral infection.
   Aspect 192. The method of claim Aspect 191, wherein the first treatment period is days 1-2 following diagnosis of the viral infection or presentation for preventing the viral infection.
   Aspect 193. The method of claim Aspect 191, wherein the first treatment period is day 1 following diagnosis of the viral infection or presentation for preventing the viral infection.
   Aspect 194. The method of any one of claims Aspect 190-Aspect 193, wherein the loading dose is about 1.1-fold to about 10-fold the treatment dose.
   Aspect 195. The method of claim Aspect 194, wherein the loading dose is about 1.5-fold to about 5-fold the treatment dose.
   Aspect 196. The method of claim Aspect 194, wherein the loading dose is about 1.5-fold to about 2.5-fold the treatment dose.
   Aspect 197. The method of any one of claims Aspect 190-Aspect 196, wherein the loading dose is administered once daily, two times daily, three times daily, or four times daily.
   Aspect 198. The method of any one of claims Aspect 190-Aspect 196, wherein the loading dose is administered at least twice daily.
   Aspect 199. The method of claim Aspect 197 or claim Aspect 198, wherein the loading dose divided equally among the number of times administered daily.
   Aspect 200. The method of any one of claims Aspect 190-Aspect 199, wherein the compound has a structure selected from:

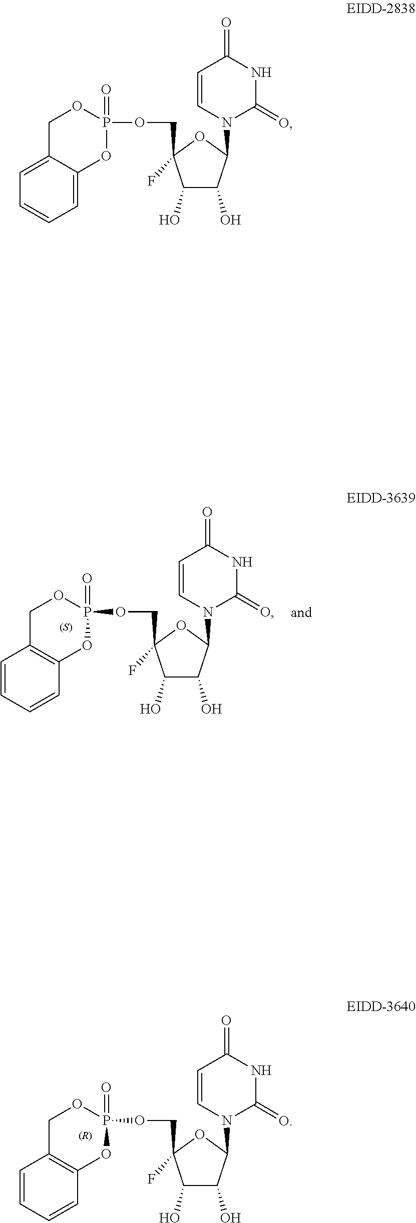

Aspect 201. The method of claim Aspect 200, wherein the compound has a structure:

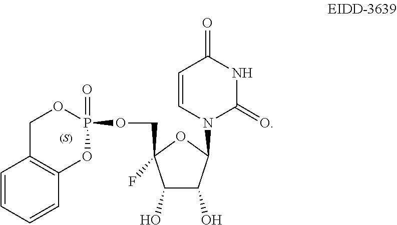

Aspect 202. The method of claim Aspect 200, wherein the compound has a structure:

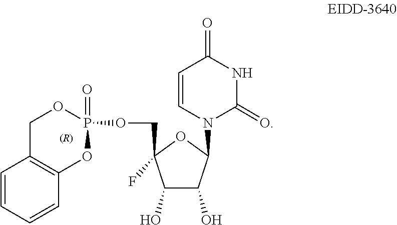

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

### Example 1. Conjugate Preparation

Mono and diphosphate prodrugs have been prepared by several groups. See Jessen et al., Bioreversible Protection of Nucleoside Diphosphates, Angewandte Chemie-International Edition English 2008, 47 (45), 8719-8722, hereby incorporated by reference. In order to prevent rupture of the P—O—P anhydride bond, one utilizes a pendant group that fragments rapidly (e.g. bis-(4-acyloxybenzyl)-nucleoside diphosphates (BAB-NDP) that is deacylated by an endogenous esterase) to generate a negative charge on the second phosphate. See also Routledge et al., Synthesis, Bioactivation and Anti-HIV Activity of 4-Acyloxybenzyl-bis(nucleosid-5′-yl) Phosphates, Nucleosides & Nucleotides 1995, 14 (7), 1545-1558 and Meier et al., Comparative study of bis(benzyl)phosphate triesters of 2′,3′-dideoxy-2′,3′-didehydrothymidine (d4T) and cycloSal-d4TMP-hydrolysis, mechanistic insights and anti-HIV activity, Antiviral Chemistry and Chemotherapy 2002, 13,101-114, both hereby incorporated by reference. Once this ° Ccurs, the P—O—P anhydride bond is less susceptible to cleavage and the remaining protecting group can then do its final unraveling to produce the nucleoside diphosphate.

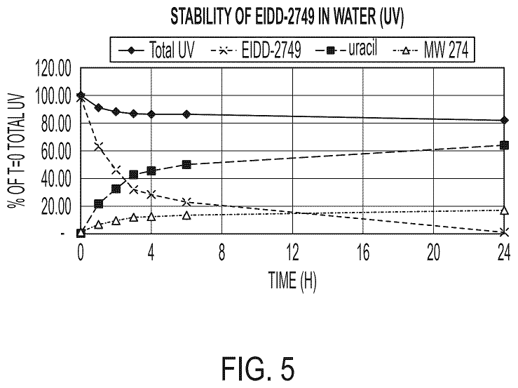

Other methods to prepare diphosphate and monothiodiphosphate prodrugs are shown in FIG. 5. Standard coupling conditions are used to prepare sphingolipid-nucleoside monophosphate prodrugs. The corresponding diphosphate prodrugs may be prepared according to the protocols shown in FIG. 5 and as provided in Smith et al., Substituted Nucleotide Analogs. U.S. Patent Application 2012/0071434; Skowronska et al., Reaction of Oxophosphorane-Sulfenyl and Oxophosphorane-Selenenyl Chlorides with Dialkyl Trimethylsilyl Phosphites—Novel Synthesis of Compounds Containing a Sulfur or Selenium Bridge Between 2 Phosphoryl Centers, Journal of the Chemical Society-Perkin Transactions 1 1988, 8, 2197-2201; Dembinski et al., An Expedient Synthesis of Symmetrical Tetra-Alkyl Mono-thiopyrophosphates, Tetrahedron Letters 1994, 35 (34), 6331-6334; Skowronska et al., Novel Synthesis of Symmetrical Tetra-Alkyl Monothiophosphates, Tetrahedron Letters 1987, 28 (36), 4209-4210; and Chojnowski et al., Methods of Synthesis of O,O-Bis TrimethylSilyl Phosphorothiolates. Synthesis-Stuttgart 1977, 10, 683-686, all hereby incorporated by reference in their entirety.

### Example 2. General Procedure for Base Coupling

The persilylated nucleobase was prepared in a round bottom flask charged with dry nucleobase (15.5 mmol), chlorotrimethylsilane (12.21 mmol), and bis(trimethylsilyl)amine (222 mmol) under nitrogen. The mixture was refluxed with stirring overnight (16 h) until all solids dissolved. The mixture was cooled to room temperature and volatiles were removed by rotary evaporation followed by high vacuum to give persilylated nucleobase. This compound was used immediately in the next step.

The freshly prepared persilylated nucleobase (15.50 mmol) was dissolved in 1,2-dichloroethane (50 mL) or chlorobenzene (50 mL) under nitrogen with stirring at room temperature. A solution of β-D-ribofuranose 1,2,3,5-tetraacetate (7.75 mmol) in 1,2-dichloroethane (50 mL) or chlorobenzene (50 mL) was added all at once to the stirred mixture.

To this mixture was added SnCl₄ (11.63 mmol) dropwise via syringe, and the mixture was stirred at room temperature 6 h until all starting material was consumed. The mixture was cooled to 0° C. and a sat. aq. NaHCO₃ solution (125 mL) was added. The mixture was warmed to room temperature and stirred 30 min. The mixture was extracted with EtOAc (2×200 mL) and the combined organic layers were washed with brine (1×100 mL), dried over Na₂SO₄, filtered, and concentrated by rotary evaporation to give 5.5 g crude product. The crude material was taken up in dichloromethane, immobilized on Celite, and subjected to flash chromatography to provide the desired acetate protected product. The ribonucleoside was deprotected using the general deprotection conditions.

### Example 3. General Cytosine Analog Coupling

In a flask charged with N⁴-benzoyl protected cytosine analog (0.793 mmol) was added bis(trimethylsilyl)amine (8.45 mmol) and ammonium sulfate (0.02 mmol) under N₂. This was heated at reflux for 2 h, after cooling to rt, solvent was removed in vacuo and further dried under high vacuum for 1 h. The residue was dissolved in dry chlorobenzene (10 ml) and β-D- or β-L-ribofuranose 1,2,3,5-tetraacetate (0.53 mmol) was added. Then SnCl₄ (0.27 ml, 2.3 mmol) was added dropwise. After stirring at rt for 1 h, this was heated to 60° C. overnight. After cooling to 0° C., solid sodium bicarbonate (0.85 g) was added, followed by EtOAc (5 mL). This was allowed to stir for 15 min and then water (0.5 mL) was added slowly. The insoluble material was filtered off and washed with more EtOAc (2.5 mL). The filtrate was washed with water once, bine once, dried (Na₂SO₄) and concentrated in vacuo. The crude material was purified by SiO₂ column chromatography.

### Example 4. General Deamination Conditions

A solution of benzoyl protected cytidine ribonucleoside (1.02 mmol) in 80% aqueous AcOH (30 mL) was heated under reflux for 16 h. The solvent was then removed in vacuo and dried under high vacuum. The white solid was triturated with ether, filtered off and washed with more ether to obtain the desired product.

### Example 5. General Uracil Analog Coupling

The persilylated uracil was prepared in a round bottom flask charged with uracil (15.5 mmol), chlorotrimethylsilane (12.21 mmol), and bis(trimethylsilyl)amine (222 mmol) under nitrogen. The mixture was refluxed with stirring overnight (16 h) until all solids dissolved until a clear colorless solution formed. The mixture was cooled to room temperature and volatiles were removed by rotary evaporation followed by high vacuum to give persilylated uracil. This compound was used immediately in the next step.

The freshly prepared persilylated uracil (15.50 mmol) was dissolved in 1,2-dichloroethane (50 mL) under nitrogen with stirring at room temperature. A solution of β-D- or β-L-ribofuranose 1,2,3,5-tetraacetate (7.75 mmol) in 1,2-dichloroethane (50 mL) was added all at once to the stirred mixture.

To this mixture was added SnCl₄ (11.63 mmol) dropwise via syringe, and the mixture was stirred at room temperature 6 h until all starting material was consumed. The mixture was cooled to 0° C. and a sat. aq. NaHCO₃ solution (125 mL) was added. The mixture was warmed to 5 room temperature and stirred 30 min. The mixture was extracted with EtOAc (2×200 mL) and the combined organic layers were washed with brine (1×100 mL), dried over Na₂SO₄, filtered, and concentrated by rotary evaporation to give 5.5 g crude product. The crude material was taken up in dichloromethane, immobilized on Celite, and subjected to flash chromatography on the Combiflash (120 g column, 5 to 50% EtOAc in hexanes gradient) to provide the product.

### Example 6. General Acetate or Benzoyl Deprotection Conditions

Benzoyl protected ribonucleoside analog (0.25 mmol) was stirred with 7 N ammonia in MeOH at rt for 15.5 h. The solvent was then removed and the crude material was purified by SiO2 column chromatography to obtain the desired ribonucleoside.

### Example 7. Synthesis of 1′-Deuterated Nucleoside Analogs

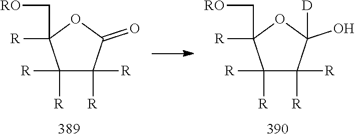

The lactone (0.0325 mol) was added to a dry flask under an argon atmosphere and was then dissolved in dry THF (250 mL). The solution as then cooled to −78° C. and a DIBAL-D solution in toluene (0.065 mol) was dropwise. The reaction was allowed to stir at −78° C. for 3-4 hours. The reaction was then quenched with the slow addition of water (3 mL). The reaction was then allowed to stir while warming to room temperature. The mixture was then diluted with two volumes of diethyl ether and was then poured into an equal volume of saturated sodium potassium tartrate solution. The organic layer was separated, dried over MgSO₄, filtered, and concentrated under reduced pressure. The residue was purified on silica eluting with hexanes/ethyl acetate. The resulting lactol was then converted to an acetate or benzolyate and subjected to base coupling conditions to introduce the desired nucleobase.

### Example 8. Synthesis of EIDD-2838

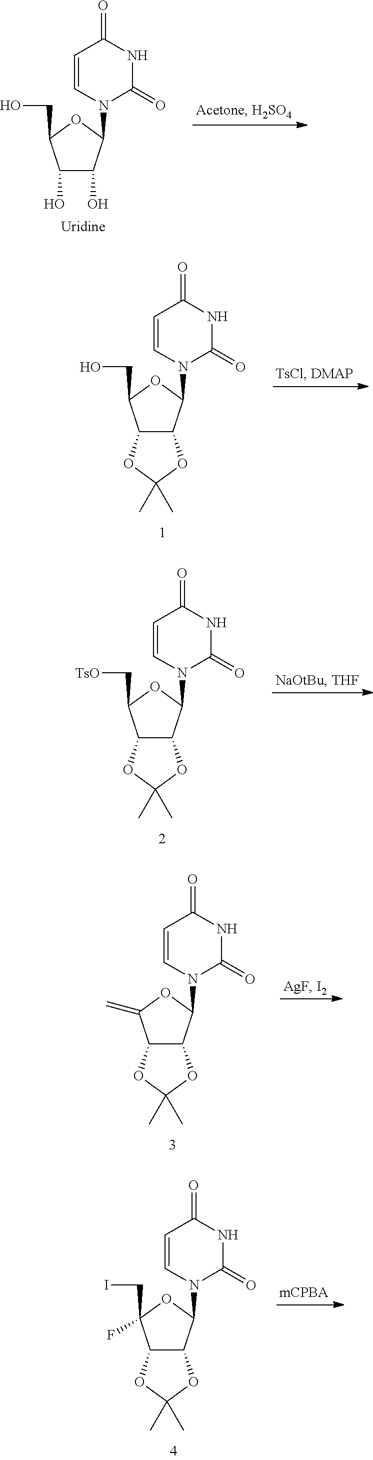 [SMILES: O=c1ccn([C@@H]2O[C@H](CO)[C@@H](O)[C@H]2O)c(=O)[nH]1 ; CC1(C)O[C@@H]2[C@H](O1)[C@@H](CO)O[C@H]2n1ccc(=O)[nH]c1=O ; Cc1ccc(S(=O)(=O)OC[C@H]2O[C@@H](n3ccc(=O)[nH]c3=O)[C@@H]3OC(C)(C)O[C@@H]32)cc1 ; C=C1O[C@@H](n2ccc(=O)[nH]c2=O)[C@@H]2OC(C)(C)O[C@H]12 ; CC1(C)O[C@H]2[C@H](n3ccc(=O)[nH]c3=O)O[C@](F)(CI)[C@H]2O1]

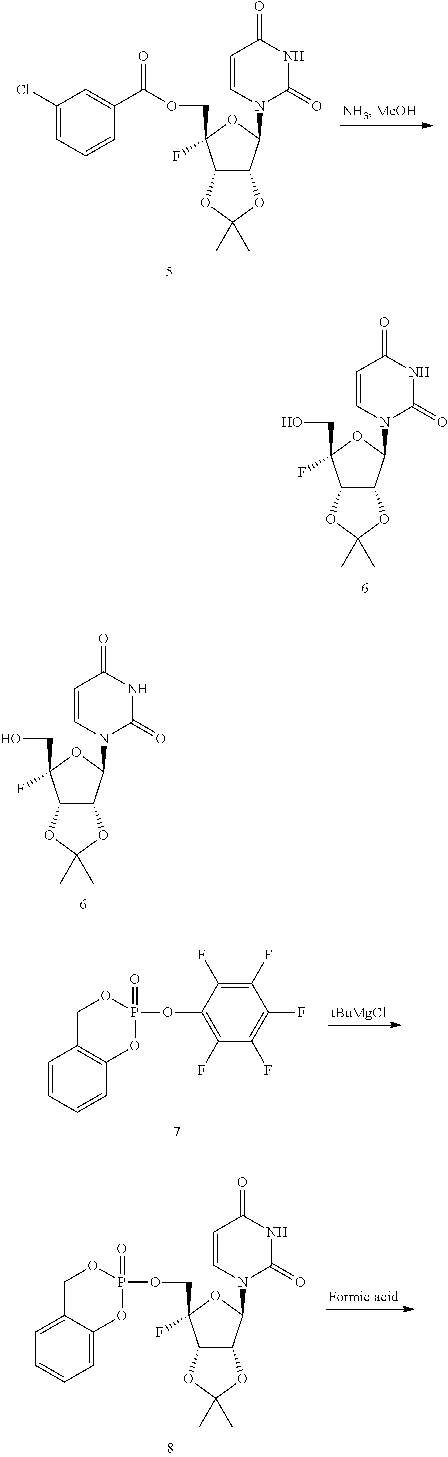

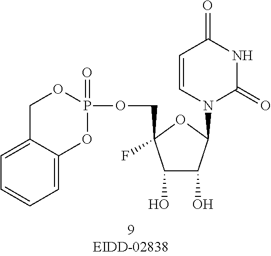 [SMILES: O=c1ccn([C@@H]2O[C@](F)(COP3(=O)OCc4ccccc4O3)[C@@H](O)[C@H]2O)c(=O)[nH]1]

A 1 L round bottom flask was charged with uridine (36.6 g, 150 mmol) and acetone (Volume: 700 ml) with stirring under nitrogen at rt. The slurry was treated with concentrated sulfuric acid (0.800 ml, 15.00 mmol) and the mixture was stirred at rt overnight. After stirring 16 h, triethylamine (41.8 ml, 300 mmol) was added all at once, the mixture was stirred 30 min, and then concentrated by rotary evaporation to give a sticky white solid. The solid was dissolved in boiling iPrOH (˜1.4 L) and allowed to cool overnight at rt. After cooling overnight, small crystals had formed. The flask was placed in the freezer for 3 h and more crystals formed. The mixture was vacuum filtered, and the solids were washed with ice-cold iPrOH (2×200 mL) and ice-cold ether (2×200 mL). The solid was recovered to give compound 1 (21.75 g, 77 mmol, 51.0% yield) as a white powdery solid.

A round bottom flask was charged with compound 1 (21.75 g, 77 mmol) and DCM (219 ml) and the mixture was stirred under nitrogen. Solid 4-DMAP (23.37 g, 191 mmol) was added all at once, and the mixture was stirred at rt until all solids dissolved. The mixture was cooled to 0° C., and tosyl chloride (17.50 g, 92 mmol) was added portionwise as a solid over 5 min. The mixture was stirred at rt for 1 h until all starting material was consumed. The mixture was transferred to a separatory funnel, and the organic layer was washed with 1 N HCl (2×200 mL), sat. aq. NaHCO₃ (1×200 mL), and brine (1×200 mL), then dried over Na₂SO₄, filtered and concentrated by rotary evaporation to give compound 2 (34.52 g, 74.8 mmol, 98% yield) as a white solid.

To a stirred solution of compound 2 (3.95 g, 9.01 mmol) in THF (30 mL) at 0° C. under nitrogen. Solid potassium tert-butoxide (3.03 g, 27.0 mmol) was added all at once, the reaction mixture turned into a yellow slurry. The mixture was stirred at 0° C. for 2 h. Silica gel (6 g) and Celite (14 g) were added along with more THF, and the mixture was concentrated by rotary evaporation. Flash chromatography on the Isco (80 g column, 1 to 5% MeOH in DCM) gave compound 3 (2.17 g, 8.15 mmol, 90% yield) as a white powdery solid.

A round bottom flask was charged with a stir bar, compound 3 (2.17 g, 8.15 mmol), silver(I) fluoride (5.17 g, 40.8 mmol), and DCM (Volume: 152 ml, Ratio: 14) at 0° C. To this vigorously stirred mixture was added a solution of iodine (4.14 g, 16.30 mmol) in THF (Volume: 10.87 ml, Ratio: 1.000) dropwise via syringe over 40 min. After addition was complete, the mixture was stirred another 15 min at 0° C., then a 1:1 mixture of sat. aq. NaHCO₃:sat. aq. Na₂S₂O₃ was added (75 mL) and the whole mixture was filtered through a Celite pad, washing with DCM (2×50 mL). The filtrates were transferred to a separation funnel, and the organic layer was dried over Na₂SO₄, filtered, and concentrated by rotary evaporation to give 4 g. Flash chromatography on the Isco (120 g column, 5 to 25% EtOAc in DCM) gave compound 4 (2.06 g, 5.00 mmol, 61.3% yield) as a pale yellow flaky solid.

A round bottom flask was charged with compound 4 (10.76 g, 26.1 mmol), tetrabutylammonium sulfate (8.86 g, 26.1 mmol), potassium hydrogen phosphate dibasic trihydrate (8.94 g, 39.2 mmol), DCM (Volume: 1088 ml, Ratio: 5) and water (Volume: 218 ml, Ratio: 1.000) and the biphasic mixture was stirred vigorously at rt. To this mixture was added solid mCPBA, 77% w/w (29.3 g, 131 mmol) all at once and the mixture was stirred at rt overnight. After stirring 20 h at rt, all SM had been consumed by TLC analysis. The mixture was quenched by slow addition of sat. aq. Na₂S₂O₃ (375 mL) followed by sat. aq. Na₂CO₃ (375 mL). The organic layer was removed, and the aqueous layer was extracted with DCM (1×450 mL). The combined organic layers were dried over Na₂SO₄, filtered, and concentrated by rotary evaporation to give 22 g crude. The crude was taken up in DCM, and flash chromatography on the Isco (330 g column, 5 to 25% EtOAc in DCM) gave 10 g of semipure product. The compound was taken up in DCM, and flash chromatography on the Isco (330 g column, 5 to 70% EtOAc in hexanes) gave compound 5 (6.91 g, 15.68 mmol, 60.0% yield) as an off-white flaky solid.

A round bottom flask was charged with compound 5 (3.53 g, 8.0 mmol) and ammonia in MeOH (34.3 ml, 240 mmol) at 0° C. The mixture was stirred for 5 h, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 6 (2.20 g, 7.28 mmol, 91% yield) as a white powdery solid.

A 1 L 3-neck RBF equipped with temperature probe, overhead stirrer and addition funnel (argon inlet) was charged with phosphorus oxychloride (15.50 ml, 166 mmol) in THF (300 ml), evacuated and purged with argon 3×, then cooled to <−70° C. using dry ice/acetone. A solution of 2-(hydroxymethyl)phenol (18.77 g, 151 mmol) and triethylamine (44.3 ml, 317 mmol) in 200 mL of THF was slowly added via addition funnel over 30 minutes. The resulting light tan mixture was slowly warmed to RT and stirred for 3 hrs. Cooled to 0° C. using an ice bath and added triethylamine (25.3 ml, 181 mmol), then slowly added a THF (100 mL) solution of 2,3,4,5,6-pentafluorophenol (25.05 g, 136 mmol) to the rapidly stirred mixture. Warmed to RT and monitored by TLC (25% EtOAc/hexanes). SM consumed in <2 hrs, only product (Rf=0.5) present. The oil was purified by SGC (glass column, 10-25% EtOAc/hexanes), fractions containing product were pooled and concentrated under reduced pressure to yield compound 7 (41.2 g, 117 mmol, 77% yield) as a white solid.

To a stirred solution of compound 6 (1.95 g, 6.45 mmol) in THF (Volume: 96 ml, Ratio: 5) at 0° C. under nitrogen, was added a solution of tert-butylmagnesium chloride, 1.0 M in THF (14.19 ml, 14.19 mmol) dropwise via syringe. A white precipitate formed; the mixture was warmed to rt and stirred for 30 min, then recooled to 0° C. A solution of compound 7 (5.68 g, 16.13 mmol) in THF (Volume: 19.20 ml, Ratio: 1.000) was added dropwise via syringe, and the mixture was warmed to rt and stirred overnight. After 18 h stirring, a little SM remained and one slightly less polar product had formed. The mixture was quenched by addition of solid NH₄Cl (2 g) and the mixture was immobilized on Celite. Flash chromatography on the Isco (220 g column, 1 to 5% MeOH in DCM) gave 1.94 g of a white solid that consisted of desired product and pentafluorophenol. The solid was taken up in DCM and washed with sat. aq. NaHCO₃ (3×100 mL). The organic layer was dried over Na₂SO₄, filtered, and concentrated by rotary evaporation to give compound 8 (1.70 g, 3.61 mmol, 56.0% yield) as a white powdery solid.

A round bottom flask was charged with compound 8 (0.250 g, 0.532 mmol) and formic acid, 80% aq. (Volume: 10 mL). The mixture was stirred at rt under nitrogen overnight. After stirring 20 h, all volatiles were removed by rotary evaporation. The residue was taken up in MeOH and immobilized on Celite. Flash chromatography on the Isco (24 g column, 1 to 15% MeOH in DCM) gave a white powdery solid, 175 mg, 90-95% pure by NMR. The white powder was taken up in a 5:1 water:MeCN mixture, and reverse phase flash chromatography on the Isco (100 g C18 column, 100% water to 100% MeCN) gave good separation of the impurity. The fractions containing desired product were concentrated, taken up in 5:1 water:MeCN, frozen in a dry ice bath, and lyophilized to provide compound 9, EIDD-2838.

### Example 9. Synthesis of Compound 17

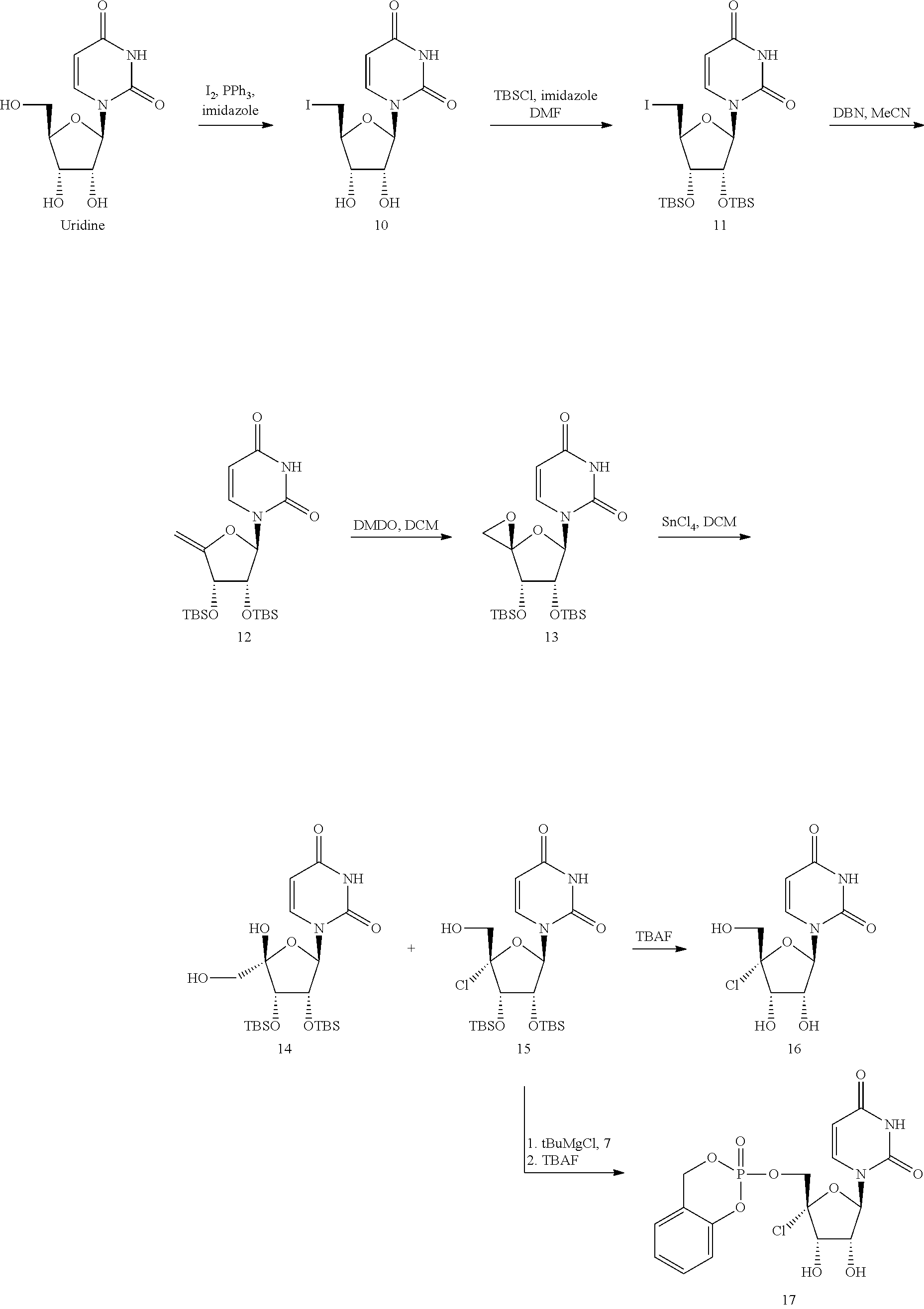

Uridine (1 mmol) was suspended in dioxane (4 mL) followed by the addition of pyridine (2 mmol), PPh₃ (1.5 mmol), and iodine (1.5 mmol) under an argon atmosphere. The mixture was stirred at room temperature overnight. The reaction mixture was quenched with methanol and saturated aqueous Na₂S₂O₃ and was then evaporated to dryness to provide crude compound 10, which was used directly in the next step.

Crude compound 10 was dissolved in dry DMF under an argon atmosphere followed by the addition of imidazole (5 equivalents) and TBSCl (4 equivalents) at 0° C. The mixture was allowed to warm to room temperature and stir overnight. The reaction mixture was partitioned between AcOEt/H₂O (3:1). The organic layer was dried over MgSO₄, filtered, and concentrated under reduced pressure. The resulting residue was purified on a silica gel column eluting with hexanes and ethyl acetate to provide compound 11.

Compound 11 was dissolved in dry MeCN and treated with DBN (2.25 equivalents) at 0° C. under an argon atmosphere. The reaction was allowed to stir overnight. The reaction mixture was neutralized with AcOH and then was evaporated to dryness. The residue was partitioned between DCM and saturated aqueous NaHCO₃. The organic layer was dried over MgSO₄, filtered, and concentrated under reduced pressure. The resulting residue was purified on a silica gel column eluting with hexanes and ethyl acetate to provide compound 12.

To a solution of compound 12 in dry DCM (20 mL/mmol 12) was added DMDO (0.1M in acetone, 1.2 equivalents) at −30° C. under an argon atmosphere. The reaction was allowed to stir for 1 hour and was then evaporated to dryness to afford compound 13, which was used immediately in the next step.

To a solution of compound 13 in dry DCM (20 mL/mmol 13) was added SnCl₄ (3 equivalents) at −30° C. under an argon atmosphere. The misture was allowed to stir for 1 hour and was then quenched with saturated aqueous NaHCO₃. The mixture was filtered through a celite pad, and the filtrate was partitioned between DCM and saturated aqueous NaHCO₃. The organic layer was dried over MgSO₄, filtered, and concentrated under reduced pressure. The resulting residue was purified on a silica gel column eluting with hexanes and ethyl acetate to provide compounds 14 and 15 in a 2:1 ratio.

Compound 15 was treated with TBAF (2.5 equivalents) in THF. After starting material was consumed, the reaction mixture was concentrated under reduced pressure and purified by reverse phase to obtain compound 16.

Compound 15 was treated under the same conditions as compound 6 followed by treatment with TBAF to obtain compound 17.

### Example 10. Synthesis of EIDD-2749

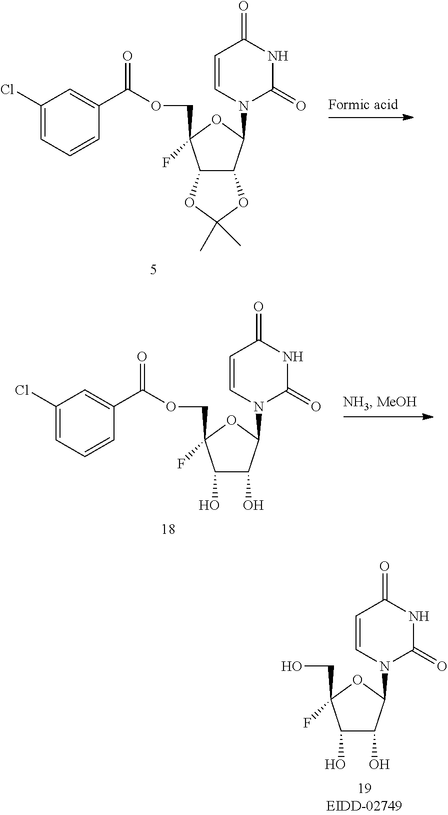 [SMILES: CC1(C)O[C@H]2[C@H](n3ccc(=O)[nH]c3=O)O[C@](F)(COC(=O)c3cccc(Cl)c3)[C@H]2O1 ; O=C(OC[C@@]1(F)O[C@@H](n2ccc(=O)[nH]c2=O)[C@H](O)[C@@H]1O)c1cccc(Cl)c1 ; O=c1ccn([C@@H]2O[C@](F)(CO)[C@@H](O)[C@H]2O)c(=O)[nH]1]

A round bottom flask was charged with compound 5 (0.250 g, 0.567 mmol) and formic acid, 80% aq. (Volume: 10 mL). The mixture was stirred at room temperature under nitrogen overnight. After stirring 20 h, all volatiles were removed by rotary evaporation. The residue was taken up in MeOH and immobilized on Celite. Flash chromatography on the Isco (24 g column, 1 to 15% MeOH in DCM) gave a white powdery solid 90-95% pure by NMR. The white powder was taken up in a 5:1 water:MeCN mixture, and reverse phase flash chromatography on the Isco (100 g C18 column, 100% water to 100% MeCN) gave good separation of the impurity. The fractions containing desired product were concentrated, taken up in 5:1 water:MeCN, frozen in a dry ice bath, and lyophilized to provide compound 18.

A round bottom flask was charged with compound 18 (3.53 g, 8.8 mmol) and ammonia in MeOH (34.3 ml, 240 mmol) at 0° C. The mixture was allowed to stir for 5 hours, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 19, EIDD-2749, (2.20 g, 7.28 mmol, 91% yield) as a white powdery solid.

### Example 11. Synthesis of Compound 25

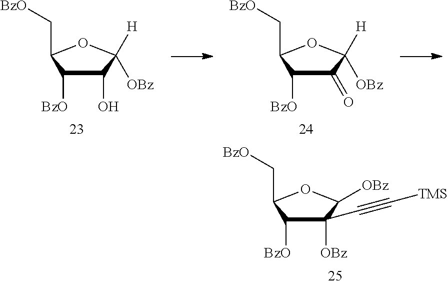

To a stirred solution of DMP (27.5 g, 64.9 mmol) in DCM (162 mL, 0.2M) was cooled to 0° C. and 23 (15 g, 32.4 mmol) was added. The reaction was stirred at 0° C. and allowed to warm to room temperature. After stirring for 18 hours the reaction mixture was concentrated under reduced pressure to a past which was then slurried in 100 mL ethyl ether followed by filtration through a 50 g pad of silica/mag sulfate 1:1 by mass and washed with a total of 400 mL ethyl ether. The ether layer was washed with 2.5 g of sodium thiosulfate in 15 mL water then 2×30 mL cooled sodium bicarbonate, and finally with 30 mL brine. The filtrate was then dried over sodium sulfate, filtered, and concentrated under reduced pressure to provide a foam which was used without further purification. Before use in the next step, a solution of ketone (32.6 mmol) in DCM (200 mL) was prepared and stirred overnight over 5 g of magnesium sulfate at room temperature. After 18 hours of stirring, the solution was filtered and concentrated under reduced pressure.

To a −78° C. solution of TMS Ethylene (11.4 mL, 80 mmol) in dry THF (100 mL) under argon was added butyl lithium (30.5 mL, 2.5M hexanes, 76 mmol). After 30 minutes of stirring, lithiated alkyne was cannulated into a −78° C. suspension of anhydrous CeCl₃ (33.5 g, 90 mmol, dried overnight 150° C. under high vacuum) in dry THF (130 mL) with 2×15 mL rinses of THF. After 90 minutes of stirring, a solution of 24 (32.4 mmol) in dry THF (50 mL) was added via cannula (2×10 mL rinse THF). After 3 hours of stirring, the resulting solution was quenched with saturated aqueous ammonium chloride (100 mL). The reaction was warmed to room temperature and filtered through a celite pad. The celite pad was washed with ethyl ether (3×100 mL) and with saturated aqueous ammonium chloride (100 mL). The filtrate was separated and the organics were washed with saturated aqueous ammonium chloride (100 mL) and brine (100 mL). The filtrate was dried over sodium sulfate, filtered, and concentrated under reduced pressure to provide an oil which was purified by silica gel chromatography 10-50% ethyl acetate in hexanes to provide the product as a mixture of anomers.

To a stirred 0° C. solution of the above product (32.4 mmol) in dry DCM (163 mL, 0.2M) under argon was added sequentially triethyl amine (18 mL, 130 mmol) DMAP (3.98 g, 32.4 mmol), and benzoyl chloride (9.46 mL, 82 mmol). After stirring for 16 hours, the reaction was concentrated under reduced pressure and then slurried in 200 mL ethyl ether and filtered. The organics were concentrated under reduced pressure to provide a paste which was purified by silica gel chromatography eluting with 10-25% ethyl acetate in hexanes to provide 25 as a mixture of anomers. Compound 25 can then be subjected to general base coupling conditions followed by the appropriate deprotection conditions.

### Example 12. Synthesis of Compound 29

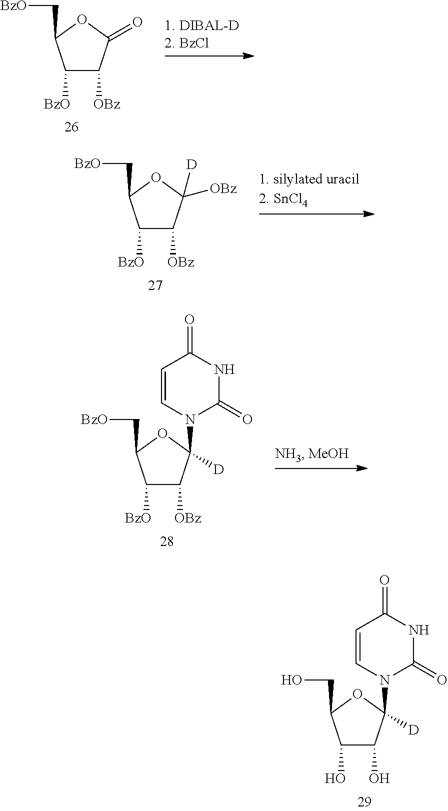

The lactone (0.0325 mol) was added to a dry flask under an argon atmosphere and was then dissolved in dry THF (250 mL). The solution was then cooled to −78° C. and a DIBAL-D solution in toluene (0.065 mol) was added dropwise. The reaction was allowed to stir at −78° C. for 3-4 hours. The reaction was then quenched with the slow addition of water (3 mL). The reaction was then allowed to stir while warming to room temperature. The mixture was then diluted with two volumes of diethyl ether and was then poured into an equal volume of saturated sodium potassium tartrate solution. The organic layer was separated, dried over MgSO₄, filtered, and concentrated under reduced pressure. The residue was purified on silica eluting with hexanes/ethyl acetate. The resulting lactol, as a solution in dry DCM, was then treated with benzoyl chloride, trimethylamine, and DMAP. The reaction was allowed to stir at 0° C. until all the strating material was consumed. Next, the reaction mixture was washed with water and then brine. The organic layer was dried over MgSO₄, filtered, and concentrated under reduced pressure. The product was purified on silica eluting with hexanes/ethyl acetate.

To a stirred suspension of uracil (3.92 g, 2 eq) in HMDS (18 mL) was added ammonium sulfate (230 mgs, 0.1 eq). The suspension was then refluxed 18 h to obtain a clear solution. The solution was cooled to room temperature and concentrated under reduced pressure to a paste. Sugar 27 was dissolved in 1,2-dichloroethane (120 mL) and concentrated under reduced pressure to about 80 mL. The sugar solution was then cannulated into the flask containing silylated base with 2×20 mL rinses of DCE. The reaction was cooled to 0° C. and then tin tetrachloride was added dropwise over 5 minutes. After 30 minutes of stirring, the reaction was allowed to warm to room temperature and was stirred for a further 18 hours overnight. The reaction was charged with 10 g sodium bicarbonate and 10 g celite. 10 mL saturated aqueous sodium bicarbonate was added dropwise (gas evolution ° Ccured). After the quench, the reaction was allowed to stir 30 minutes and then was filtered through a celite pad. The pad was washed with DCM (2×150 mL) and the combined organics were washed with 100 mL saturated aqueous sodium bicarbonate. The organics were collected, dried over sodium sulfate, filtered and concentrated under reduced pressure to provide a brown paste that was purified by silica gel chromatography eluting with 25-100% ethyl aceate in hexanes.

A round bottom flask was charged with compound 28 and ammonia in MeOH at 0° C. The mixture was allowed to stir for 5 hours, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 29.

### Example 13. Synthesis of Compound 37

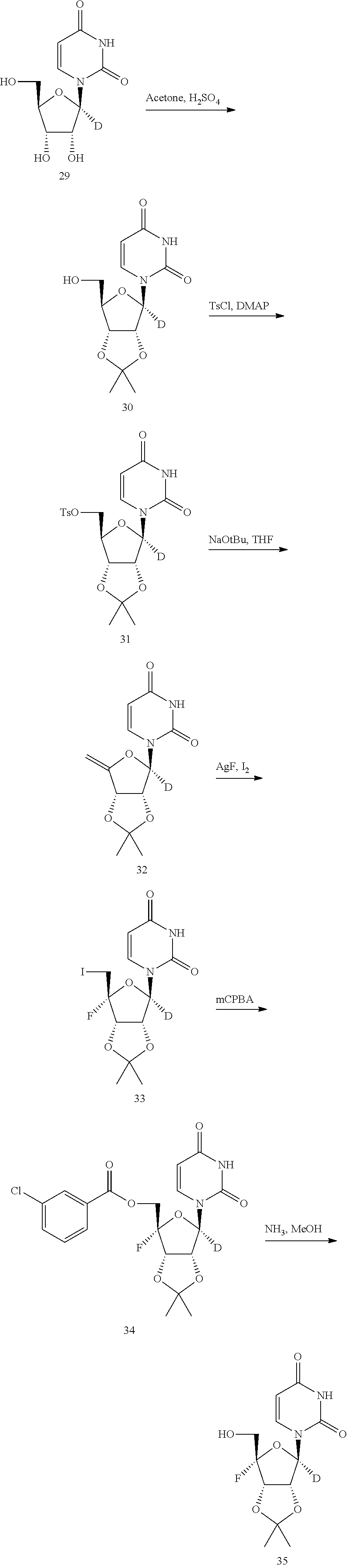

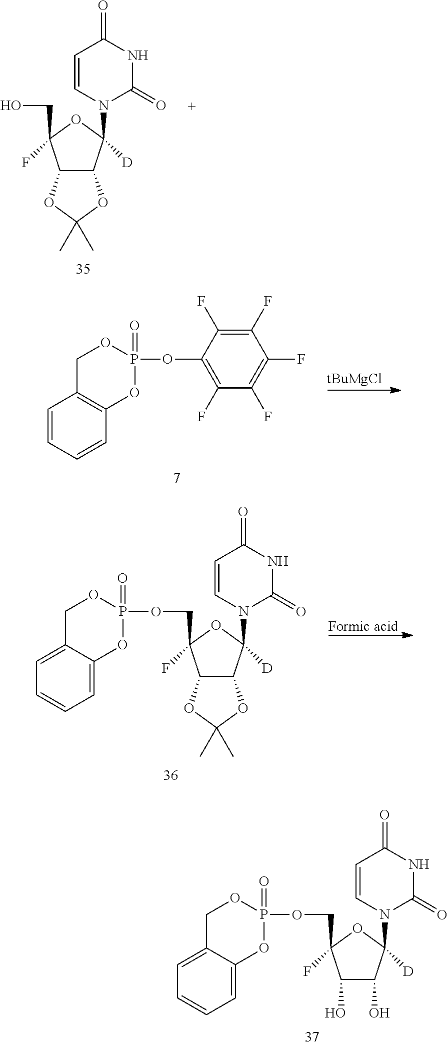

A 1 L rbf was charged with compound 29 (36.6 g, 150 mmol) and acetone (Volume: 700 ml) with stirring under nitrogen at rt. The slurry was treated with concentrated sulfuric acid (0.800 ml, 15.00 mmol) and the mixture was stirred at rt overnight. After stirring 16 h, triethylamine (41.8 ml, 300 mmol) was added all at once, the mixture was stirred 30 min, and then concentrated by rotary evaporation to give a sticky white solid. The solid was dissolved in boiling iPrOH (˜1.4 L) and allowed to cool overnight at rt. After cooling overnight, small crystals had formed. The flask was placed in the freezer for 3 h and more crystals formed. The mixture was vacuum filtered, and the solids were washed with ice-cold iPrOH (2×200 mL) and ice-cold ether (2×200 mL). The solid was recovered to give compound 30 (21.75 g, 77 mmol, 51.0% yield) as a white powdery solid.

A round bottom flask was charged with compound 30 (21.75 g, 77 mmol) and DCM (219 ml) and the mixture was stirred under nitrogen. Solid 4-DMAP (23.37 g, 191 mmol) was added all at once, and the mixture was stirred at rt until all solids dissolved. The mixture was cooled to 0° C., and tosyl chloride (17.50 g, 92 mmol) was added portionwise as a solid over 5 min. The mixture was stirred at rt for 1 h until all starting material was consumed. The mixture was transferred to a separatory funnel, and the organic layer was washed with 1 N HCl (2×200 mL), sat. aq. NaHCO₃ (1×200 mL), and brine (1×200 mL), then dried over Na₂SO₄, filtered and concentrated by rotary evaporation to give compound 31 (34.52 g, 74.8 mmol, 98% yield) as a white solid.

To a stirred solution of compound 31 (3.95 g, 9.01 mmol) in THF (30 mL) at 0° C. under nitrogen. Solid potassium tert-butoxide (3.03 g, 27.0 mmol) was added all at once, the reaction mixture turned into a yellow slurry. The mixture was stirred at 0° C. for 2 h. Silica gel (6 g) and Celite (14 g) were added along with more THF, and the mixture was concentrated by rotary evaporation. Flash chromatography on the Isco (80 g column, 1 to 5% MeOH in DCM) gave compound 32 (2.17 g, 8.15 mmol, 90% yield) as a white powdery solid.

A round bottom flask was charged with a stir bar, compound 32 (2.17 g, 8.15 mmol), silver(I) fluoride (5.17 g, 40.8 mmol), and DCM (Volume: 152 ml, Ratio: 14) at 0° C. To this vigorously stirred mixture was added a solution of iodine (4.14 g, 16.30 mmol) in THF (Volume: 10.87 ml, Ratio: 1.000) dropwise via syringe over 40 min. After addition was complete, the mixture was stirred another 15 min at 0° C., then a 1:1 mixture of sat. aq. NaHCO₃:sat. aq. Na₂S₂O₃ was added (75 mL) and the whole mixture was filtered through a Celite pad, washing with DCM (2×50 mL). The filtrates were transferred to a separation funnel, and the organic layer was dried over Na₂SO₄, filtered, and concentrated by rotary evaporation to give 4 g. Flash chromatography on the Isco (120 g column, 5 to 25% EtOAc in DCM) gave compound 33 (2.06 g, 5.00 mmol, 61.3% yield) as a pale yellow flaky solid.

A round bottom flask was charged with compound 33 (10.76 g, 26.1 mmol), tetrabutylammonium sulfate (8.86 g, 26.1 mmol), potassium hydrogen phosphate dibasic trihydrate (8.94 g, 39.2 mmol), DCM (Volume: 1088 ml, Ratio: 5) and water (Volume: 218 ml, Ratio: 1.000) and the biphasic mixture was stirred vigorously at rt. To this mixture was added solid mCPBA, 77% w/w (29.3 g, 131 mmol) all at once and the mixture was stirred at rt overnight. After stirring 20 h at rt, all SM had been consumed by TLC analysis. The mixture was quenched by slow addition of sat. aq. Na₂S₂O₃ (375 mL) followed by sat. aq. Na₂CO₃ (375 mL). The organic layer was removed, and the aqueous layer was extracted with DCM (1×450 mL). The combined organic layers were dried over Na₂SO₄, filtered, and concentrated by rotary evaporation to give 22 g crude. The crude was taken up in DCM, and flash chromatography on the Isco (330 g column, 5 to 25% EtOAc in DCM) gave 10 g of semipure product. The compound was taken up in DCM, and flash chromatography on the Isco (330 g column, 5 to 70% EtOAc in hexanes) gave compound 34 (6.91 g, 15.68 mmol, 60.0% yield) as an off-white flaky solid.

A round bottom flask was charged with compound 34 (3.53 g, 8.0 mmol) and ammonia in MeOH (34.3 ml, 240 mmol) at 0° C. The mixture was stirred for 5 h, at which point all starting material was consumed. The mixture was concentrated by rotary evaporation to give ˜4 g crude as a yellow oil. The crude was taken up in DCM, and flash chromatography on the Isco (120 g column, 1 to 5% MeOH in DCM) gave compound 35 (2.20 g, 7.28 mmol, 91% yield) as a white powdery solid.

A 1 L 3-neck RBF equipped with temperature probe, overhead stirrer and addition funnel (argon inlet) was charged with phosphorus oxychloride (15.50 ml, 166 mmol) in THF (300 ml), evacuated and purged with argon 3×, then cooled to <−70° C. using dry ice/acetone. A solution of 2-(hydroxymethyl)phenol (18.77 g, 151 mmol) and triethylamine (44.3 ml, 317 mmol) in 200 mL of THF was slowly added via addition funnel over 30 minutes. The resulting light tan mixture was slowly warmed to RT and stirred for 3 hrs. Cooled to 0° C. using an ice bath and added triethylamine (25.3 ml, 181 mmol), then slowly added a THF (100 mL) solution of 2,3,4,5,6-pentafluorophenol (25.05 g, 136 mmol) to the rapidly stirred mixture. Warmed to RT and monitored by TLC (25% EtOAc/hexanes). SM consumed in <2 hrs, only product (Rf=0.5) present. The oil was purified by SGC (glass column, 10-25% EtOAc/hexanes), fractions containing product were pooled and concentrated under reduced pressure to yield compound 7 (41.2 g, 117 mmol, 77% yield) as a white solid.

To a stirred solution of compound 35 (1.95 g, 6.45 mmol) in THF (Volume: 96 ml, Ratio: 5) at 0° C. under nitrogen, was added a solution of tert-butylmagnesium chloride, 1.0 M in THF (14.19 ml, 14.19 mmol) dropwise via syringe. A white precipitate formed; the mixture was warmed to rt and stirred for 30 min, then recooled to 0° C. A solution of compound 7 (5.68 g, 16.13 mmol) in THF (Volume: 19.20 ml, Ratio: 1.000) was added dropwise via syringe, and the mixture was warmed to rt and stirred overnight. After 18 h stirring, a little SM remained and one slightly less polar product had formed. The mixture was quenched by addition of solid NH₄Cl (2 g) and the mixture was immobilized on Celite. Flash chromatography on the Isco (220 g column, 1 to 5% MeOH in DCM) gave 1.94 g of a white solid that consisted of desired product and pentafluorophenol. The solid was taken up in DCM and washed with sat. aq. NaHCO₃ (3×100 mL). The organic layer was dried over Na₂SO₄, filtered, and concentrated by rotary evaporation to give compound 36 (1.70 g, 3.61 mmol, 56.0% yield) as a white powdery solid.

A round bottom flask was charged with compound 36 (0.250 g, 0.532 mmol) and formic acid, 80% aq. (Volume: 10 mL). The mixture was stirred at rt under nitrogen overnight. After stirring 20 h, all volatiles were removed by rotary evaporation. The residue was taken up in MeOH and immobilized on Celite. Flash chromatography on the Isco (24 g column, 1 to 15% MeOH in DCM) gave a white powdery solid, 175 mg, 90-95% pure by NMR. The white powder was taken up in a 5:1 water:MeCN mixture, and reverse phase flash chromatography on the Isco (100 g C18 column, 100% water to 100% MeCN) gave good separation of the impurity. The fractions containing desired product were concentrated, taken up in 5:1 water:MeCN, frozen in a dry ice bath, and lyophilized to provide compound 37.

### Example 14. Synthesis of Compounds 40 and 41

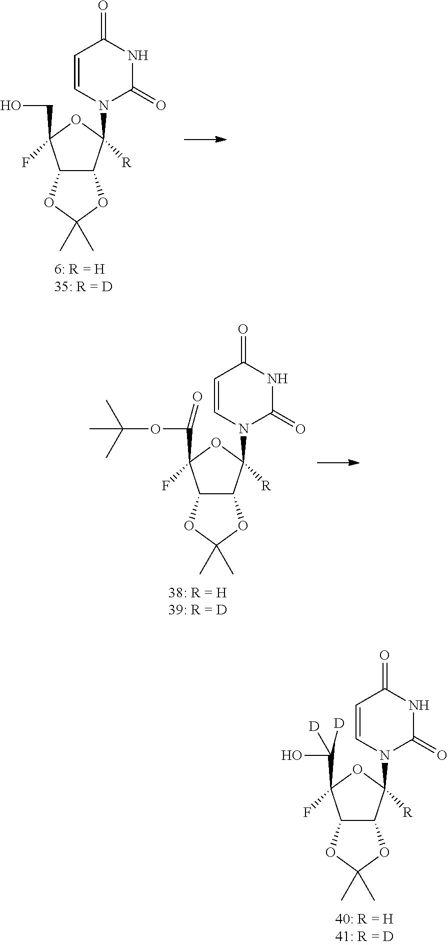

Either the nucleoside 6 or 35 was suspended in methylene chloride (40 mL, partially soluble). After stirring at rt for 30 min the mixture was treated sequentially with PDC, acetic anhydride and then tert-butanol. The mixture was allowed to stir at room temperature. TLC (5% methanol in DCM) and LCMS indicated only a small amount of remaining starting material at 4 hours. The mixture was filtered through a pad of silica gel that was loaded into a 150 mL fritted funnel. The silica was eluted with ethyl acetate. The collected filtrate was concentrated by under reduced pressure. The crude dark oil was purified by chromatography over silica gel (25 mm×175 mm) with 2:1 hexanes:ethyl acetate to ethyl acetate gradient. The pure fractions were collected and concentrated under reduced pressure to give of a white gum. The material was placed under high vacuum for 2 days to provide either compound 38 or 39. The material was used in the next step without further purification.

The 5′-protected nucleoside 38 or 39 was dissolved in 200 proof ethanol and was then treated with solid sodium borodeuteride. The mixture became homogeneous and was then heated to 80° C. After 12 h, a white/pale yellow precipitate formed. The mixture was allowed to cool to rt. TLC (5% methanol in methylene chloride) indicates complete conversion of starting material. The mixture was cooled to 0° C. with an ice-bath and then slowly quenched with acetic acid (approximately 1 mL). The clear solution was warmed to rt and then partitioned between ethyl acetate (30 mL) and brine (3 mL). The organic phase was concentrated and then purified by chromatography over silica gel (19 mm×180 mm) using a mobile phase of 5% methanol in methylene chloride to provide compound 40 or 41. Compounds 40 and 41 can then be deprotected to obtain the unprotected ribonucleoside using 80% formic acid as described previously. Additionally, Compounds 40 and 41 can be conjugated to prodrug reagent 7 followed by deprotection as described previously.

### Example 15. Synthesis of Compound 124

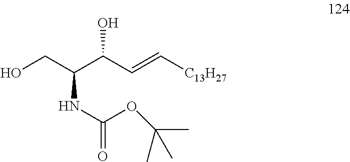

Prepared according to Boumendjel, Ahcene and Miller, Stephen Journal of Lipid Research 1994, 35, 2305.

A mixture of sphingosine (450 mg, 1.50 mmol) and di-tert-butyl dicarbonate (0.656 g, 3.01 mmol) in methylene chloride (100 mL) at 4° C. was treated dropwise with diisopropylethylamine (0.53 mL, 3.01 mmol). After gradual warming to rt, the mixture was stirred for an additional 12 h and then diluted with methylene chloride (100 mL) followed by a wash with water (30 mL) and brine (30 mL). The organic phase was dried over sodium sulfate, filtered and concentrated to dryness. The crude residue was purified by flash column chromatography over silica gel (19 mm×175 mm) using 50% ethyl acetate in hexanes to give N-tert-butyloxycarbonyl-sphingosine (540 mg, 90%) as a white solid.

¹H NMR (300 MHz, Chloroform-d) δ 5.77 (dt, J=15.4, 8.4 Hz, 1H), 5.52 (dd, J=15.4, 8.4 Hz, 1H), 3.93 (dd, J=11.4, 3.7 Hz, 1H), 3.70 (dd, J=11.4, 3.7 Hz, 1H), 3.59 (s, 3H), 2.05 (q, J=7.0 Hz, 2H), 1.52 (s, 9H), 1.25 (s, 22H), 0.87 (t, J=6.5 Hz, 3H).

### Example 16. Synthesis of Compound 125

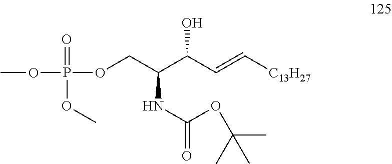

N-tert-Butyloxycarbonyl-sphingosine 124(540 mg, 1.35 mmol) was rendered anhydrous by co-evaporation with anhydrous pyridine (2×12 mL). The residue was then dissolved in anhydrous pyridine and treated with carbon tetrabromide (622 mg, 1.88 mmol). The mixture was cooled to 0° C. and treated dropwise with a solution of trimethylphosphite (0.25 mL, 2.10 mmol) in anhydrous pyridine (3 mL) over a 30 min period. After an additional 12 h at rt, both LCMS and tlc (5% methanol in methylene chloride) analysis indicated complete conversion. The mixture was quenched with water (2 mL) and then concentrated to dryness. The resulting dark oil was dissolved in ethyl acetate (150 mL) and washed with 3% HCL solution (2×20 mL) followed by saturated sodium bicarbonate solution (30 mL). The organic layer was dried over sodium sulfate, filtered and concentrated. The crude residue was purified by flash column chromatography over silica gel (19 mm×175 mm) using 2% methanol in methylene chloride to give N-tert-butyloxycarbonyl-sphingosine-1-O-dimethylphosphate 125 (350 mg, 51%) as a gum.

¹H NMR (400 MHz, Chloroform-d) δ 5.82 (dt, J=15.4, 7.1 Hz, 1H), 5.48 (dd, J=15.4, 7.1 Hz, 1H), 4.99 (d, J=8.9 Hz, 1H), 4.32 (ddd, J=10.7, 8.0, 4.6 Hz, 1H), 4.11 (ddt, J=10.7, 7.4, 3.1 Hz, 2H), 3.77 (dd, J=11.1, 2.1 Hz, 6H), 2.01 (q, J=7.1 Hz, 2H), 1.41 (s, 9H), 1.34 (m, 2H), 1.23 (m, 20H), 0.86 (t, J=6.4 Hz, 3H).

³¹P NMR (162 MHz, Chloroform-d) δ 2.00.

MS C17H25NO4 [M+Na+]; calculated: 330.2, found: 330.2.

### Example 17. Synthesis of Compound 126

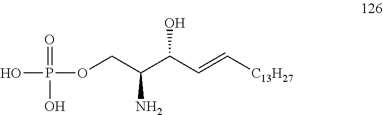

A solution of N-tert-butyloxycarbonyl-sphingosine-1-O-dimethylphosphate 125 (350 mg, 0.689 mmol) in anhydrous methylene chloride (8 mL) was treated dropwise with trimethylsilyl bromide (0.45 mL, 3.45 mmol) at 0° C. After warming to room temperature, the mixture was allowed to stir at rt for 6 h and then concentrated to dryness. The resulting residue was co-evaporated with methylene chloride to remove excess trimethylsilyl bromide and then treated with 66% aqueous THF (6 mL). The resulting precipitate was collected by filtration to give sphingosine-1-phosphate 126 (218 mg, 83%) as a white solid.

¹H NMR (400 MHz, Methanol-d₄+CD₃CO₂D) δ 5.84 (dt, J=15.5, 6.7 Hz, 1H), 5.46 (dd, J=15.5, 6.7 Hz, 1H), 4.33 (t, J=6.0 Hz, 1H), 4.13 (ddd, J=11.8, 7.7, 3.6 Hz, 1H), 4.03 (dt, J=11.8, 8.4 Hz, 1H), 3.47 (ddd, J=8.3, 4.8, 3.2 Hz, 1H), 2.10-1.99 (m, 2H), 1.37 (m, 2H), 1.24 (m, 20H), 0.83 (t, J=6.4 Hz, 3H).

³¹P NMR (162 MHz, Chloroform-d) δ 0.69.

MS C₁₈H₃₈NO₅P [M−H⁺]; calculated: 378.2, found: 378.2.

### Example 18. Synthesis of Compound 131

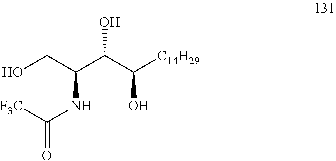

To a slurry of phytosphingosine (4 g, 12.6 mmol) and anhydrous powdered potassium carbonate (5.22 g, 37.8 mmol) in methylene chloride (85 mL) was added trifluoroacetic anhydride (1.96 mL, 13.9 mmol). The mixture was stirred at rt for 18 h and then diluted with methylene chloride (500 mL). The mixture was washed with water (100 mL). Methanol (60 mL) was added to break the emulsion. The organic phase was then dried over sodium sulfate, filtered and concentrated to give 131 (4.9 g, 94%) as a white solid

¹H NMR (400 MHz, DMSO-d₆) δ 8.90 (s, 1H), 4.90-4.68 (m, 1H), 4.56 (d, J=6.1 Hz, 1H), 4.43 (s, 1H), 3.97 (d, J=7.6 Hz, 1H), 3.65 (d, J=10.8 Hz, 1H), 3.46 (t, J=10.2 Hz, 1H), 3.32-3.16 (m, 1H), 1.42 (tt, J=15.7, 7.5 Hz, 2H), 1.20 (s, 24H), 0.83 t, J=6.8 Hz, 3H).

### Example 19. Synthesis of Compound 132

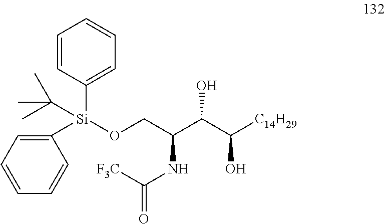

N-Trifluoroacetyl-phytosphingosine (131, 1.88 g, 4.5 mmol) in anhydrous pyridine (23 mL) was treated with DMAP (56 mg, 0.45 mmol) and then dropwise with tert-butyldiphenylsilyl chloride (1.38 g, 5.0 mmol). After 18 h concentrated to dryness. The resulting residue was dissolved in ethyl acetate (200 mL) and washed with saturated ammonium chloride (2×50 mL) and then brine (50 mL). The aqueous phases was back-extracted with ethyl acetate (50 mL). Combined organic phases were dried over sodium sulfate and concentrated to give crude 1-O-tert-Butyldiphenylsilyl-2-N-trifluoroacetyl-phytosphingosine 132 (3 g, 100%) as a gum. The material was used in the next step without further purification.

¹H NMR (400 MHz, Chloroform-d) δ 7.62 (m, 2H), 7.60-7.56 (m, 2H), 7.47-7.31 (m, 6H), 7.07 (d, J=8.4 Hz, 1H), 4.23 (dd, J=8.5, 4.1 Hz, 1H, 4.04 (dt, J=11.0, 2.5 Hz, 1H), 3.82 (ddd, J=11.0, 4.3, 1.8 Hz, 1H), 3.64 (dq, J=10.6, 6.0, 4.3 Hz, 2H), 1.45 (m, 2H), 1.39-1.15 (m, 24H), 1.05 (m, 9H), 0.94-0.80 (t, J=6.9 Hz 3H).

### Example 20. Synthesis of Compound 133

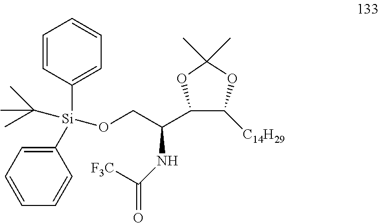

A solution of 1-O-tert-Butyldiphenylsilyl-2-N-trifluoroacetyl-phytosphingosine 132 (3 g, 4.5 mmol) in 1/1 (v/v) 2,2-dimethoxypropane/THF was treated with catalytic amount of p-toluenesulfonic acid (87 mg, 0.45 mmol) and allowed to stir for 16 h at rt. The mixture was quenched with saturated sodium bicarbonate (30 mL) and then excess THF/2,2-dimethoxypropane was removed under vacuum. The mixture was extracted with ethyl acetate (200 mL). After washing with brine, the organic layer was dried over sodium sulfate, filtered and concentrated. The crude oil was purified by column chromatography (25 mm×175 mm) over silica gel with a hexanes/ethyl acetate mobile phase to give 133(2.45 g, 78%).

¹H NMR (400 MHz, Chloroform-d) δ 7.68-7.63 (m, 2H), 7.63-7.57 (m, 2H), 7.39 (m, 6H), 6.54 (d, J=9.4 Hz, 1H), 4.23 (dd, J=8.2, 5.6 Hz, 1H), 4.12 (ddd, J=13.3, 6.9, 3.8 Hz, 2H), 3.96 (dd, J=10.5, 3.9 Hz, 1H), 3.69 (dd, J=10.5, 2.9 Hz, 1H), 1.52-1.36 (m, 2H), 1.33 (s, 3H), 1.31 (s, 3H), 1.24 (m, 24H), 1.03 (s, 9H), 0.86 (t, J=53.7, 6.9 Hz, 3H).

### Example 21. Synthesis of Compound 134

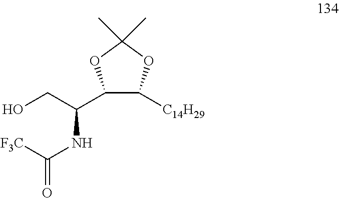

A solution of 1-O-tert-Butyldiphenylsilyl-3,4-O-isopropylidene-2-N-trifluoroacetyl-phytosphingosine 133 (2.45 g, 3.54 mmol) in THF (18 mL) was treated with tetrabutylammonium fluoride (4.25 mL of a 1.0 M solution in THF, 4.25 mmol) and stirred at rt for 12 h. The mixture was diluted with ethyl acetate (100 mL) and saturated ammonium chloride (2×50 mL) and then brine (50 mL). The organic phase was dried over sodium sulfate, filtered, and concentrated to give a white solid that was further purified by column chromatography (25 mm×175 mm) over silica gel with a 9:1 hexanes:ethyl acetate mobile phase to afford 134(1.5 g, 93%) as a white solid.

¹H NMR (300 MHz, Chloroform-d) δ 6.92 (d, J=8.7 Hz, 1H), 4.31-4.16 (m, 2H), 4.11 (dq, J=11.7, 3.7 Hz, 1H), 4.00 (dd, J=11.5, 2.6 Hz, 1H), 3.70 (dd, J=11.5, 3.6 Hz, 1H), 1.48 (s, 3H), 1.35 (s, 3H), 1.25 (m, 26H), 0.88 (t, J=6.9 Hz 3H).

### Example 22. Synthesis of Compound 135

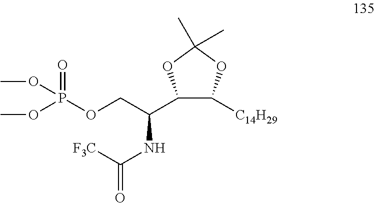

A solution of 3,4-O-Isopropylidene-2-N-Trifluoroacetyl-phytosphingosine 134(630 mg, 1.39 mmol) was rendered anhydrous by co-evaporation with anhydrous pyridine (2×12 mL). The residue was then dissolved in anhydrous pyridine (12 mL) and treated with carbon tetrabromide (533 mg, 1.67 mmol). The mixture was cooled to 0° C. and treated dropwise with a solution of trimethylphosphite (0.23 mL, 1.95 mmol) in anhydrous pyridine (3 mL) over a 30 min period. After an additional 12 h at rt, both LCMS and tlc (5% methanol in methylene chloride) analysis indicated complete conversion. The mixture was quenched with water (2 mL) and then concentrated to dryness. The resulting dark oil was dissolved in ethyl acetate (100 mL) and washed with 3% HCL solution (2×20 mL) followed by saturated sodium bicarbonate solution (30 mL). The organic layer was dried over sodium sulfate, filtered and concentrated. The crude residue was purified by flash column chromatography over silica gel (19 mm×175 mm) using 2% methanol in methylene chloride to give 135 (650 mg, 83%).

¹H NMR (300 MHz, Chloroform-d) δ 7.42 (d, J=8.8 Hz, 1H), 4.36 (td, J=10.9, 5.0 Hz, 1H), 4.25 (m, 1H), 4.19 (m, J=6.5, 2.0 Hz, 3H), 3.77 (dd, J=11.2, 7.5 Hz, 6H), 1.44 (s, 3H), 1.33 (s, 3H), 1.25 (m, 26H), 0.87 (t, J=6.6 Hz, 3H).

³¹P NMR (121 MHz, Chloroform-d) δ 1.69.

MS C₂₅H₄₇F₃NO₇P [M−H⁺]; calculated: 560.3, found: 560.2.

### Example 23. Synthesis of Compound 136 (3,4-O-Isopropylidene-2-N-trifluoroacetyl-phytosphingosine-1-phosphate)

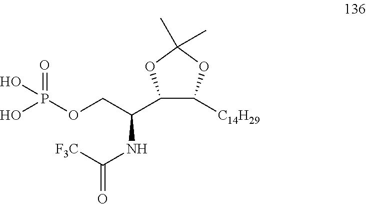

A solution of 3,4-O-Isopropylidene-2-N-trifluoroacetyl-phytosphingosine-1-O-dimethylphosphate 135 (650 mg, 1.16 mmol) in anhydrous methylene chloride (12 mL) was treated dropwise with trimethylsilyl bromide (0.81 mL, 6.23 mmol) at 0° C. After 12 h at rt, the mixture was concentrated to dryness and the resulting residue co-evaporated with methylene chloride (3×50 mL) to remove excess trimethylsilyl bromide. The residue then was dissolved in cold (4° C.) solution of 1% NH₄OH while maintaining pH 7-8. After 10 min at rt, the mixture was concentrated to dryness, and the resulting solid triturated with methanol/acetonitrile. The solid was collected by filtration, washed with acetonitrile, and dried under high vacuum to give 136 (500 mg, 75%) as a white solid.

¹H NMR (300 MHz, Methanol-d₄) δ 4.31 (dd, J=8.7, 5.4 Hz, 1H), 4.09 (m, 4H), 1.42 (s, 3H), 1.36 (s, 3H), 1.31 (m, 26H), 0.89 (t, J=6.4 Hz, 3H).

³¹P NMR (121 MHz, Methanol-d₄) δ 1.28.

¹⁹F NMR (282 MHz, Methanol-d₄) δ −77.13.

HRMS C₂₃H₄₂F₃NO₇P [M−H⁺]; calculated: 532.26565, found: 532.26630.

### Example 24. Synthesis of Compound 137

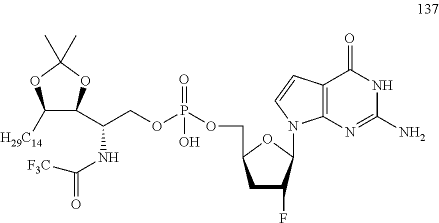

A mixture of N-trifluoroacetyl-phytosphingosine-1-phosphate 136(200 mg, 0.373 mmol) and 2′,3′-dideoxy-2′-fluoro-7-deazaguanine (100 mg, 0.373 mmol) was rendered anhydrous by co-evaporation with anhydrous pyridine (3×10 mL). The resulting residue then was dissolved in anhydrous pyridine (4 mL) and treated with diisopropylcarbodiimide (127 mg, 1.01 mmol) and HOBt (60 mg, 0.447 mmol). After 24 h at 75° C., the reaction mixture was cooled to rt and concentrated to dryness. The crude material was purified by flash column chromatography (19 mm×170 mm) over silica gel using a solvent gradient from 5 to 7.5% methanol in chloroform with 1% (v/v) NH₄OH to give 137(80 mg, 27%) as a white solid.

¹H NMR (300 MHz, Methanol-d₄) δ 6.88 (d, J=3.8 Hz, 1H), 6.46 (d, J=3.8 Hz, 1H), 6.24 (d, J=19.9 Hz, 1H), 5.34 (dd, J=52.4, 4.6 Hz, 1H), 4.53 (s, 1H), 4.34-3.97 (m, 6H), 2.63-2.17 (m, 2H), 1.40 (s, 3H), 1.30 (s, 3H), 1.27 (m, 26H), 0.89 (t, J=6.6 Hz, 3H).

³¹P NMR (121 MHz, Methanol-d₄) δ 12.50.

¹⁹F NMR (282 MHz, Methanol-d₄) δ −77.10, −179.69-−180.25 (m).

MS C₃₄H₅₂2F₄N₅O₉P [M−H⁺]; calculated: 781.3, found: 782.2.

### Example 25. Experimental Procedure for Synthesis of Prodrugs

A solution of isopropyl 2-((chloro(phenoxy)phosphoryl)amino)propanoate (0.397 g, 1.300 mmol) in anhydrous THF (5 ml) was added to a −78° C. stirred solution of 2′-deoxy-2′-fluoronucleoside (0.812 mmol) and 1-methyl-1H-imidazole (0.367 ml, 4.63 mmol) in pyridine (10.00 ml). After 15 min the reaction was allowed to warm to room temperature and was stirred for an additional 3 hours. Next, the solvent was removed under reduced pressure. The crude product was dissolved in 120 ml of DCM and was washed with 20 ml 1 N HCl solution followed by 10 ml water. The organic phase was dried over sodium sulfate, filtered and concentrated in vacuo. The residues were separated over silica column (neutralized by TEA) using 5% MeOH in DCM as a mobile phase to yield the respective products as diastereomers.

### Example 26. Synthesis of Compound 174 (N-tert-Butyloxycarbonyl-phytosphingosine)

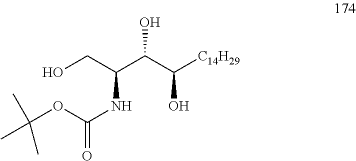

A suspension of phytosphingosine (10.6 g, 33.5 mmol) and triethylamine (5.6 ml, 40.2 mmol) in THF (250 mL) was treated dropwise with di-tert-butyl dicarbonate (8.6 mL, 36.9 mmol). After 12 h at rt, the mixture was concentrated to dryness and the resulting white solid was recrystallized from ethyl acetate (80 mL) and then dried under high vacuum at 35° C. for 12 h to give 174(10.5 g, 75%).

¹H NMR (400 MHz, Chloroform-d) δ 5.31 (d, J=8.5 Hz, 1H), 3.89 (d, J=11.1 Hz, 1H), 3.83 (s, 2H), 3.74 (dd, J=11.1, 5.2 Hz, 1H), 3.65 (d, J=8.3 Hz, 1H), 3.61 (d, J=3.9 Hz, 1H), 1.43 (s, 9H), 1.23 (s, 27H), 0.86 (t, J=6.4 Hz, 3H).

### Example 27. Synthesios of Compound 175 (2-O-tert-Butyldiphenylsilyl-1-N-tert-butyloxycarbonyl-phytosphingosine)

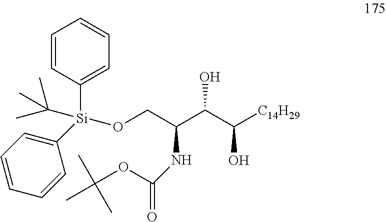

A solution of N-tert-Butyloxycarbonyl-phytosphingosine 174 (9.5 g, 22.65 mmol) and triethylamine (3.8 mL, 27.2 mmol) in anhydrous methylene chloride/DMF (120 mL/10 mL) was treated dropwise with tert-butylchlorodiphenylsilane (7 mL, 27.25 mmol). After 18 h at rt, the mixture was diluted with methylene chloride (200 mL) and washed with 0.2N HCl (100 mL) and then brine (100 mL). The organic phase was dried over sodium sulfate, filtered and then concentrated to give 175 (14.9 g) as an oil which was used in the next reaction without further purification.

¹H NMR (400 MHz, Chloroform-d) δ 5.31 (d, J=8.5 Hz, 1H), 3.89 (d, J=11.1 Hz, 1H), 3.83 (m, 1H), 3.74 (dd, J=11.1, 5.2 Hz, 1H), 3.65 (d, J=8.3 Hz, 1H), 3.61 (d, J=3.9 Hz, 1H), 1.43 (s, 9H), 1.23 (s, 27H), 0.86 (t, J=6.4 Hz, 3H).

### Example 28. Synthesios of Compound 176 (2-O-tert-Butyldiphenylsilyl-1-N-tert-butyloxycarbonyl-3,4-O-isopropylidene-phytosphingosine)

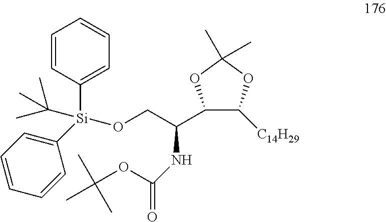

A solution of 2-O-tert-Butyldiphenylsilyl-1-N-tert-butyloxycarbonyl-phytosphingosine (175, 14.9 g, 22.65 mmol) in 1/1 (v/v) THF/2,2-dimethoxypropane was treated with catalytic para-toluenesulfonic acid (860 mg, 4.53 mmol). After 24 h, the mixture was quenched with saturated sodium bicarbonate solution (50 mL). The mixture was concentrated and then dissolved in ethyl acetate (200 mL) and washed with brine (2×50 mL). The organic phase was dried over sodium sulfate, filtered and concentrated to give 176 (15.7 g) as a gum which was used in the next step without further purification.

¹H NMR (400 MHz, Chloroform-d) δ 7.66 (m, 4H), 7.51-7.27 (m, 6H), 4.78 (d, J=10.0 Hz, 1H), 4.18 (dd, J=9.3, 5.5 Hz, 1H), 3.89 (dd, J=9.9, 3.3 Hz, 1H), 3.80 (d, J=9.9 Hz, 1H), 3.72 (d, J=9.9 Hz, 1H), 1.45 (s, 9H), 1.42 (s, 3H), 1.35 (s, 3H), 1.25 (s, 27H), 1.05 (s, 9H), 0.87 (t, J=6.5 Hz, 3H).

### Example 29. Synthesios of Compound 177 (1-N-tert-butyloxycarbonyl-3,4-O-isopropylidene-phytosphingosine)

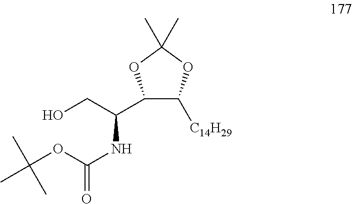

A solution of 2-O-tert-Butyldiphenylsilyl-1-N-tert-butyloxycarbonyl-3,4-O-isopropylidene-phytosphingosine 176 (15.7 g, 22.6 mmol) in THF at 0° C. was treated dropwise with a solution of tetrabutylammonium fluoride (1.0 M in THF, 24.9 mL, 24.9 mmol) over a 20 min period. After 16 h at rt, tlc (3:1 hexanes:ethyl acetate) indicated complete conversion. The mixture was concentrated to dryness and the resulting residue was dissolved in ethyl acetate (300 mL) and washed with water (3×100 mL). The organic phase was dried over sodium sulfate, filtered and concentrated. The resulting oil purified by flash column chromatography (35 mm×180 mm) using a solvent gradient from 25 to 50% ethyl acetate in hexanes to give 177 (7.3 g, 71% over 3 steps) as a white solid.

¹H NMR (400 MHz, Chloroform-d) δ 4.93 (d, J=9.1, 1H), 4.16 (q, J=7.1, 6.4 Hz, 1H), 4.07 (t, J=6.5 Hz, 1H), 3.83 (dd, J=11.1, 2.4 Hz, 1H), 3.76 (m, 1H), 3.67 (dd, J=11.2, 3.6 Hz, 1H), 1.43 (s, 3H), 1.42 (s, 9H), 1.32 (s, 3H), 1.23 (s, 27H), 0.86 (t, J=6.9 Hz, 3H).

### Example 30. General Procedure for the Preparation of 5′-Phosphoramidate Prodrugs

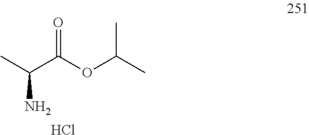

Synthesis of chlorophosphoramidate: Thionyl chloride (80 g, 49.2 mL, 673 mmol) was added dropwise over a 30 min period to a suspension of L-alanine (50 g, 561 mmol) in isopropanol (500 mL). The mixture was heated to a gentle reflux for 5 h and then concentrated by rotary evaporator (bath set at 60° C.). The resulting thick gum solidified upon trituration with ether (150 ml). The white powder was triturated a second time with ether (150 mL), collected by filtration while under a stream of argon, and then dried under high vacuum for 18 h to give (S)-isopropyl 2-aminopropanoate hydrochloride (88 g, 94%).

¹H NMR (400 MHz, DMSO-d₆) δ 8.62 (s, 3H), 5.10-4.80 (m, 1H), 3.95 (q, J=7.2 Hz, 1H), 1.38 (d, J=7.2 Hz, 3H), 1.22 (d, J=4.6 Hz, 3H), 1.20 (d, J=4.6 Hz, 3H).

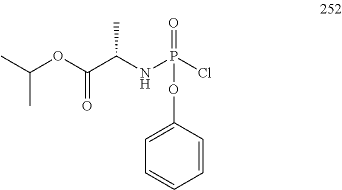 [SMILES: CC(C)OC(=O)[C@H](C)NP(=O)(Cl)Oc1ccccc1]

A solution of phenyl dichlorophosphate (30.9 g, 146 mmol) in dichloromethane (450 mL) was cooled to 0° C. then treated with (S)-isopropyl 2-aminopropanoate hydrochloride (24.5 g, 146 mmol). The mixture was further cooled to −78° C. and then treated dropwise with triethylamine (29.6 g, 40.8 mL, 293 mmol) over a 30 min period. The mixture continued to stir at −78° C. for an additional 2 h and then allowed to gradually warm to rt. After 18 h the mixture was concentrated to dryness and the resulting gum dissolved in anhydrous ether (150 mL). The slurry was filtered while under a stream of argon, and the collected solid washed with small portions of anhydrous ether (3×30 mL). Combined filtrates were concentrated to dryness by rotary evaporator to give a 1:1 diastereomeric mixture of phosphochloridate (41.5 g, 93%) as pale yellow oil.

¹H NMR (300 MHz, Chloroform-d) δ 7.43-7.14 (m, 5H), 5.06 (m, 1H), 4.55 (dd, J=14.9, 7.0 Hz, 1H), 4.21-4.01 (m, 1H), 1.48 (d, J=7.0 Hz, 2H), 1.27 (d, J=6.2 Hz, 3H), 1.26 (d, J=5.8 Hz, 3H).

³¹P NMR (121 MHz, Chloroform-d) δ 8.18 and 7.87.

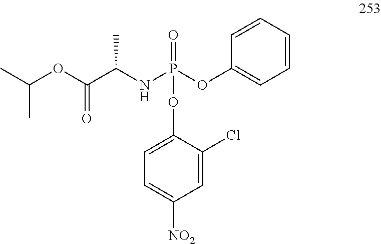 [SMILES: CC(C)OC(=O)[C@H](C)NP(=O)(Oc1ccccc1)Oc1ccc([N+](=O)[O-])cc1Cl]

A solution of phenyl dichlorophosphate (60 g, 42.5 mL, 284 mmol) in dichloromethane (300 mL) was cooled to 0° C. and then treated with (S)-isopropyl 2-aminopropanoate hydrochloride (47.7 g, 284 mmol). The mixture was further cooled to −78° C. and treated dropwise with a solution of triethylamine (57.6 g, 79 mL, 569 mmol) in methylene chloride (300 mL) over a 1 h period. The reaction mixture was warmed to 0° C. for 30 min and then treated with a preformed mixture of 2-chloro-4-nitrophenol (46.9 g, 270 mmol) and triethylamine (28.8 g, 39.6 mL, 284 mmol) in dichloromethane (120 mL) over a 20 min period. After 2 h at 0° C., the mixture was filtered through a fritted funnel, and the collected filtrate concentrated to dryness. The crude gum was dissolved MTBE (500 mL) and washed with 0.2 M K₂CO₃ (2×100 mL) followed by 10% brine (3×75 mL). The organic phase was dried over sodium sulfate, filtered and concentrated to dryness by rotary evaporator to give a diastereomeric mixture (100 g, 93%) as a pale yellow oil.

¹H NMR (400 MHz, Chloroform-d) δ 8.33 (dd, J=2.7, 1.1 Hz, 1H, diastereomer 1), 8.31 (dd, J=2.7, 1.1 Hz, 1H, diastereomer 2), 8.12 (dd, J=9.1, 2.7 Hz, 1H), 7.72 (dt, J=9.1, 1.1 Hz, 1H), 7.40-7.31 (m, 2H), 7.28-7.19 (m, 6H), 5.01 (pd, J=6.3, 5.2 Hz, 1H), 4.22-4.08 (m, 1H), 3.96 (td, J=10.7, 9.1, 3.6 Hz, 1H), 1.43 (dd, J=7.0, 0.6 Hz, 3H), 1.40 (dd, J=7.2, 0.6 Hz, 3H, diastereomer 2), 1.25-1.20 (m, 9H).

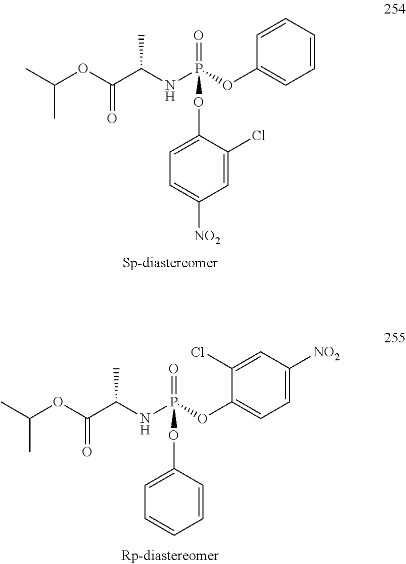 [SMILES: CC(C)OC(=O)[C@H](C)N[P@](=O)(Oc1ccccc1)Oc1ccc([N+](=O)[O-])cc1Cl ; CC(C)OC(=O)[C@H](C)N[P@@](=O)(Oc1ccccc1)Oc1ccc([N+](=O)[O-])cc1Cl]

Separation of compound 253 diastereomers: The diastereomeric mixture 253 (28 g, 63.2 mmol) was dissolved in 2:3 ethyl acetate:hexanes (100 mL) and cooled to −20° C. After 16 h, the resulting white solid was collected by filtration and dried under high vacuum to give a 16:1 Sₚ:Rₚ-diastereomeric mixture (5.5 g, 19.6%). The mother liquor was concentrated and the resulting residue dissolved in 2:3 ethyl acetate:hexanes (50 mL). After 16 h at −10° C., the resulting white solid was collected and dried under high vacuum to give a 1:6 Sₚ:Rₚ-diastereomeric mixture (4 g, 14%). The 16:1 Sₚ:Rₚ-diastereomeric mixture (5.5 g, 12.4 mmol) was suspended in hot hexanes (50 mL) and treated slowly with ethyl acetate (approximately 10 mL) until complete dissolution. After cooling to 0° C., the resulting white solid was collected by filtration, washed with hexanes, and dried under high vacuum to give the Sₚ-diastereomer of 254 (4.2 g, 76%) as a single isomer.

¹H NMR (Sₚ-diastereomer, 400 MHz, Chloroform-d) δ 8.33 (dd, J=2.7, 1.1 Hz, 1H), 8.12 (dd, J=9.1, 2.7 Hz, 1H), 7.71 (dd, J=9.1, 1.2 Hz, 1H), 7.41-7.30 (m, 2H), 7.29-7.11 (m, 3H), 5.00 (m, 1H), 4.25-4.07 (m, 1H), 3.97 (dd, J=12.7, 9.4 Hz, 1H), 1.43 (d, J=7.0 Hz, 3H), 1.23 (d, J=2.2 Hz, 3H), 1.21 (d, J=2.2 Hz, 3H).

The 1:6 Sₚ:Rₚ-diastereomeric mixture (4 g, 12.4 mmol) was suspended in hot hexanes (50 mL) and treated slowly with ethyl acetate (approximately 5 mL) until complete dissolution. After cooling to 0° C., the resulting white solid was collected by filtration, washed with hexanes, and dried under high vacuum to give the Rₚ-diastereomer of 255 (3.2 g, 80%) as a single isomer. Absolute stereochemistry was confirmed by X-ray analysis.

¹H NMR (Rₚ-diastereomer, 400 MHz, Chloroform-d) δ 8.31 (dd, J=2.7, 1.1 Hz, 1H), 8.11 (dd, J=9.1, 2.7 Hz, 1H), 7.72 (dd, J=9.1, 1.2 Hz, 1H), 7.42-7.30 (m, 2H), 7.31-7.14 (m, 3H), 5.01 (p, J=6.3 Hz, 1H), 4.15 (tq, J=9.0, 7.0 Hz, 1H), 4.08-3.94 (m, 1H), 1.40 (d, J=7.0 Hz, 3H), 1.24 (d, J=3.5 Hz, 3H), 1.22 (d, J=3.5 Hz, 3H).

### Example 31. General Procedure for Phosphoramidate Prodrug Formation

The desired nucleoside (1 equivalent) to be converted into its 5′-phosphoramidate prodrug was dried in a vacuum oven at 50° C. overnight. The dry nucleoside is placed in a dry flask under an inert atmosphere and suspended in either dry THF or dry DCM to achieve a 0.05M solution. The flask was then cooled to 0° C., and the chlorophosphoramidate reagent (5 equivalents) was added to the suspended nucleoside. Next, 1-methylimidazole (8 equivalents) was added to the reaction mixture dropwise. The reaction was allowed to stir at room temperature for 12-72 hours. After the reaction was complete as judged by TLC, the reaction mixture was diluted with ethyl acetate. The diluted reaction mixture was then washed with saturated aqueous ammonium chloride solution. The aqueous layer was re-extracted with ethyl acetate. The combined organic layers were then washed with brine, dried over MgSO₄, filtered, and concentrated. The concentrated crude product was then purified on silica eluting with a gradient of DCM to 5% MeOH in DCM.

### Example 32. General Procedure for Preparation of 5′-Triphosphates

Nucleoside analogue was dried under high vacuum at 50° C. for 18 h and then dissolved in anhydrous trimethylphosphate (0.3 M). After addition of Proton-Sponge® (1.5 molar equiv), the mixture was cooled to 0° C. and treated dropwise with phosphoryl chloride (1.3 molar equiv) via microsyringe over a 15 min period. The mixture continued stirring at 0° C. for 4 to 6 h while being monitored by tlc (7:2:1 isopropanol:conc. NH₄OH:water). Once greater than 85% conversion to the monophosphate, the reaction mixture was treated with a mixture of bis(tri-n-butylammonium pyrophosphate) (3 molar equiv) and tributylamine (6 molar equiv) in anhydrous DMF (1 mL). After 20 min at 0° C. with monitoring by tlc (11:7:2 NH₄OH:isopropanol:water), the mixture was treated with 20 mL of a 100 mM solution of triethylammonium bicarbonate (TEAB), stirred for 1 h at rt and then extracted with ether (3×15 mL). The aqueous phase was then purified by anion-exchange chromatography over DEAE Sephadex® A-25 resin (11×200 mm) using a buffer gradient from 50 mM (400 mL) to 600 mM (400 mL) TEAB. Fractions of 10 mL were analyzed by tlc (11:7:2 NH₄OH:isopropanol:water). Triphosphate (eluted @500 mM TEAB) containing fractions were combined and concentrated by rotary evaporator (bath <25° C.). The resulting solid was reconstituted in DI water (10 mL) and concentrated by lyophilization.

### Example 33. Synthesis of (R)-2,2,2-trifluoro-N-(1-hydroxyoctadecan-2-yl)acetamide

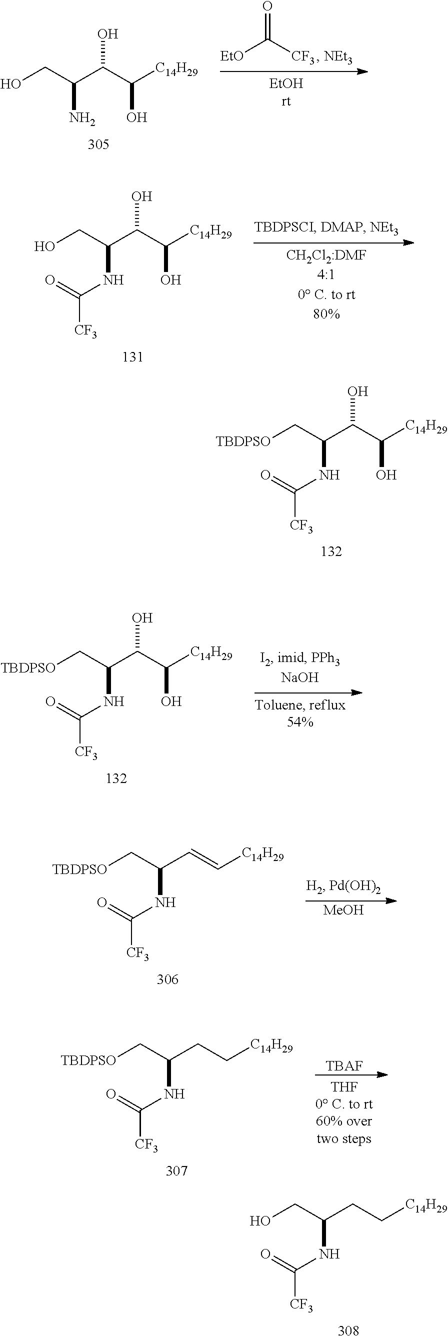

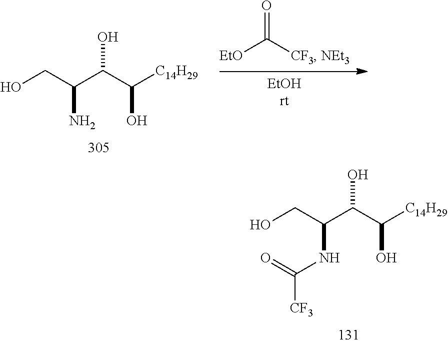

Phytosphingosine (15.75 mmol) was dissolved in EtOH (0.5M) and ethyl trifluoroacetate (15.75 mmol) was added dropwise. NEt₃ (24.41 mmol) was added next the reaction mixture stirred overnight. The solvent was removed in vacuo and the residue was taken up in EtOAc and brine, washed, dried and concentrated. The crude material that was a white powder was good enough to use in the next step without further purification. Characterization matched literature: Synthesis, 2011, 867.

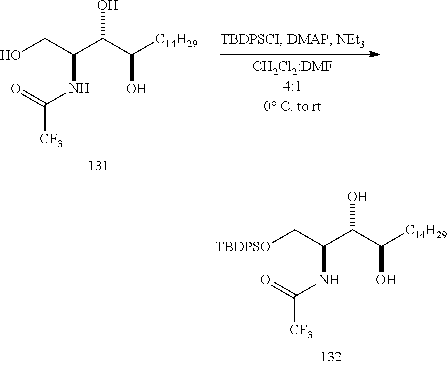

The primary alcohol (15.75 mmol), DMAP (1.575 mmol) and NEt₃ (39.4 mmol) were dissolved in CH₂Cl₂ and DMF (0.18M) mixture and cooled to 0° C. TBDPSCl (19.69 mmol) was added dropwise then the solution was allowed to warm to room temperature and stirred overnight.

NH₄Cl solution was added to quench. The reaction mixture was extracted with EtOAc and the combined organic layers were washed with water (×2) to remove DMF. It was then dried and concentrated. A column was run to purify the mixture. 10-20% EtOAc/Hex. Characterization matched literature: Synthesis, 2011, 867.

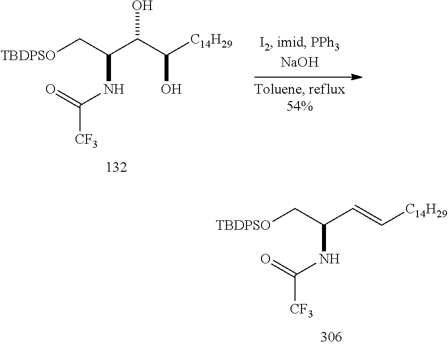

The diol (12.58 mmol), triphenylphosphine (50.3 mmol) and imidazole (50.03 mmol) were dissolved in toluene and reheated to reflux. The iodine (37.7 mmol) was then added slowly and the reaction mixture continued to be stirred at reflux. After three hours it was cooled to room temperature and 1 equivalent of iodine (12.58 mmol) was added followed by 8 equivalents of 1.5M NaOH (100.64 mmol). The reaction mixture was stirred until all the solids dissolved. The aqueous layer was removed in a separatory funnel and the organic layer was washed with Na₂S₂₀₃ solution then NaHCO₃ solution then brine. It was dried and concentrated. A column was run to purify the mixture 0-20% EtOAc/Hex and a mixture of cis and trans was obtained but carried on to the next step.

δ ¹H NMR (400 MHz, Chloroform-d) δ 7.64 (ddt, J=7.8, 3.8, 1.7 Hz, 4H), 7.51-7.35 (m, 6H), 6.68 (dd, J=16.0, 8.2 Hz, 1H), 5.6-5.40 (m, 2H), 4.57-4.46 (m, 1H), 3.84-3.62 (m, 2H), 2.04 (q, J=7.0 Hz, 1H), 1.28-1.21 (m, 24H), 1.15-0.98 (m, 9H), 0.90 (t, J=6.8 Hz, 3H).

HRMS: 617.38759.

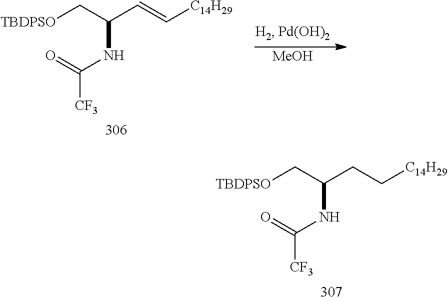

The alkene (2.91 mmol) was dissolved in MeOH (0.1M) and Pd(OH)₂/C (0.146 mmol) was added. A Parr Hydrogenator was used at 40 psi. The palladium catalyst was carefully filtered off through celite and rinsed with EtOAc. The crude material was used in the next step and provided quantitative yield.

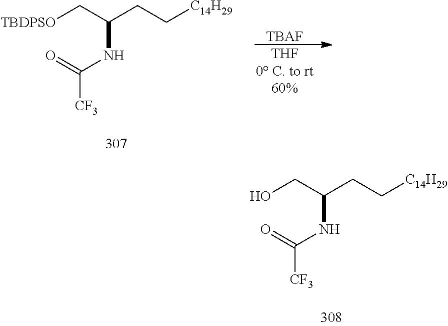

The silyl ether was dissolved in THF and cooled to 0° C. then TBAF was added dropwise. After stirring for 1 hour it was warmed to room temperature. After two hours NH₄C₁ solution was added and it was extracted with EtOAc, washed with brine and dried and concentrated. A column was run 10-50% EtOAc/Hex.

¹H NMR (400 MHz, Chloroform-d) δ 7.60 (tt, J=7.0, 1.5 Hz, 2H), 7.48-7.33 (m, 4H), 3.73 3.61 (m, 1H), 1.24 (d, J=3.5 Hz, 18H), 1.05 (s, 6H), 0.86 (t, J=6.8 Hz, 3H). HRMS: 381.28546.

### Example 34. Synthesis of Compound 380

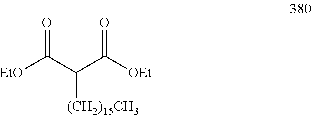

To 33.4 g sodium ethoxide solution (21% wt) in ethanol, diethyl malonate(15 g) and then 1-bromohexadecane (31.5 g) were added dropwise. After reflux for 8 hrs, ethanol was evaporated in vacuo. The remaining suspension was mixed with ice-water(200 ml) and extracted with diethyl ether (3×200 ml). The combined organic layers were dried over MgSO₄, filtered and the filtrate was evaporated in vacuo to yield a viscous oil residue. This residue was purified by column chromatography(silica: 500 g) using hexane/diethyl ether(12:1) as mobile phase to yield the main compound.

### Example 35. Synthesis of Compound 381

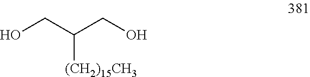

In a 250 mL round-bottomed flask was aluminum lithium hydride (2.503 g, 66.0 mmol) in Diethyl ether (90 ml) to give a suspension. To this suspension was added diethyl 2-hexadecylmalonate (18.12 g, 47.1 mmol) dropwise and the reaction was refluxed for 6 h. The reaction was followed up by TLC using PMA and H2SO4 as drying agents. The excess lithium aluminium hydride was destroyed by 200 ml of ice-water. 150 ml of 10% H2SO4 was added to dissolve aluminium hydrate. The reaction mixture was extracted by diethyl ether (100 ml×3). The organic layer including undissolved product was filtered. The collect solids were washed with ethyl acetate. The filtrate was dried over MgSO4, filtered and concentrated under reduced pressure. The product was purified on silica (100 g) column eluting with Hexane:EtOAc (3:1) to (1:1).

### Example 36. Synthesis of Compound 382

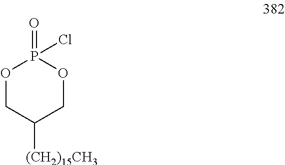

To a solution of 2-hexadecylpropane-1,3-diol (7.04 g, 23.43 mmol) in 100 ml of DCM was added dropwise phosphorous trichloride (3.59 g, 23.43 mmol) dissolved in 20 ml of DCM followed by triethylamine (6.53 ml, 46.9 mmol). The reaction was refluxed for one hour. TLC analysis showed that the starting material was consumed and two new spots formed. The mixture was concentrated to dryness, dissolved in dry diethyl ether and filtered. The filtrate was concentrated to yield the crude product (8.85 g) that was used in the next step without further purification.

### Example 37. Synthesis of 5′-Deuterated Nucleoside Analogs

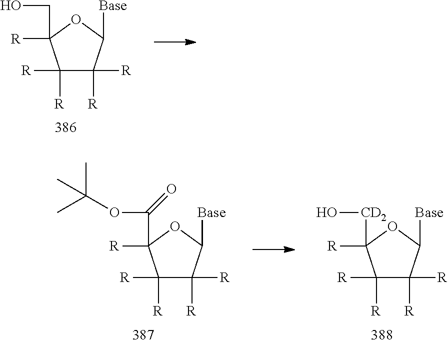

The nucleoside was suspended in methylene chloride (40 mL, partially soluble). After stirring at rt for 30 min the mixture was treated sequentially with PDC, acetic anhydride and then tert-butanol. The mixture continued to stir at room temperature. TLC (5% methanol in DCM) and LCMS indicated only a small amount of remaining starting material at 4 hours. The mixture was filtered through a pad of silica gel that was loaded into a 150 mL fritted funnel. The silica was eluted with ethyl acetate. The collected filtrate was concentrated by under reduced pressure. The crude dark oil was purified by chromatography over silica gel (25 mm×175 mm) with 2:1 hexanes:ethyl acetate to ethyl acetate gradient. The pure fractions were collected and concentrated to give of a white gum. The material was placed under high vacuum for 2 days and was used in the next step without further purification.

The 5′-protected nucleoside was dissolved in 200 proof ethanol and was then treated with solid sodium borodeuteride. The mixture became homogeneous and was then heated to 80° C. After 12 h, a white/pale yellow precipitate formed. The mixture was allowed to cool to rt. TLC (5% methanol in methylene chloride) indicates complete conversion of starting material. The mixture was cooled to 0° C. with an ice-bath and then slowly quenched with acetic acid (approximately 1 mL). The clear solution was warmed to rt and then partitioned between ethyl acetate (30 mL) and brine (3 mL). The organic phase was concentrated and then purified by chromatography over silica gel (19 mm×180 mm) using a mobile phase of 5% methanol in methylene chloride.

### Example 38. Synthesis of Compound 480

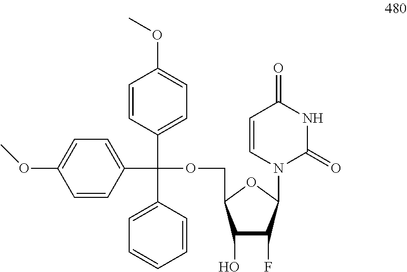

A solution of 2′-deoxy-2′-fluorouridine (6 g, 24.37 mmol) and 4,4′-(chloro(phenyl) methylene)-bis(methoxybenzene) (9.91 g, 29.2 mmol) in pyridine (48.7 ml) was stirred at rt for 16 hours. The mixture was treated with MeOH (20 mL), concentrated to dryness and was partitioned between water (50 mL) and EtOAc (250 mL). The aqueous phase was back extracted with EtOAc (50 mL) and the combined organic layers were washed with water (50 mL) and dried over Na₂SO₄. The solution was concentrated to give 2′-deoxy-2′-fluoro-5′-(4′,4′-dimethoxytrityl)uridine (14 g, quant.) which was used without further purification.

### Example 39. Synthesis of Compound 481

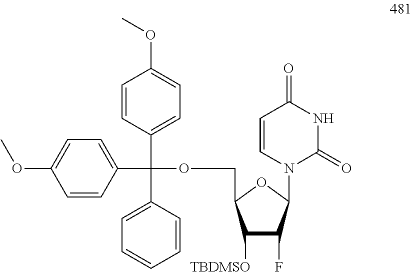

To a solution of 2′-deoxy-2′-fluoro-5′-(4′,4′-dimethoxytrityl)uridine (13.37 g, 24.37 mmol) in methylene chloride (30 mL) were added 1H-imidazole (2.48 g, 36.6 mmol) and tert-butylchlorodimethylsilane (5.51 g, 36.6 mmol). The reaction was stirred for 16 hours and then was diluted with EtOAc (250 mL). The mixture was washed with saturated aqueous sodium bicarbonate (50 mL) and brine (50 mL), dried over Na₂SO₄, filtered and concentrated to give 2′-Deoxy-2′-fluoro-3′-O-(tert-butyldimethylsilyl)-5′-(4′,4′-dimethoxytrityl)uridine (16 g, 99%). This product was used in the next step without further purification.

### Example 40. Synthesis of Compound 482

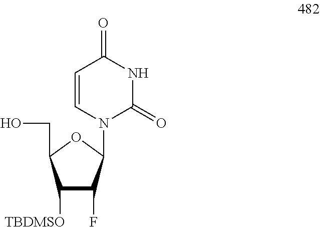

To a solution of 2′-deoxy-2′-fluoro-3′-O-(tert-butyldimethylsilyl)-5′-(4′,4′-dimethoxytrityl) uridine (13.37 g, 20.17 mmol) in DCM (10 mL) were added acetic acid (20.19 ml, 353 mmol) and water (5 ml). The reaction was stirred at room temperature for 20 hours, diluted with EtOAc (250 mL), washed with saturated aqueous NaHCO₃ (2×100 mL) and brine (100 mL), dried (sodium sulfate), filtered and concentrated. The residue was purified by column chromatography over silica gel (1% MeOH in DCM, 2% MeOH in DCM) to afford 2′-deoxy-2′-fluoro-3′-O-(tert-butyldimethylsilyl)uridine (6.73 g, 93% yield) as a yellow solid.

### Example 41. Synthesis of Compound 483

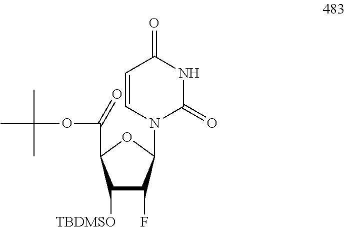

To a suspension of PDC (14.05 g, 37.3 mmol) in anhydrous DCM (37.3 ml)/DMF (9.34 ml) were added sequentially 2-methylpropan-2-ol (35.7 ml, 373 mmol), 2′-deoxy-2′-fluoro-3′-O-(tert-butyldimethylsilyl)uridine (6.73 g, 18.67 mmol) and acetic anhydride (17.62 ml, 187 mmol). After 18 hours, the mixture was quenched with absolute EtOH (5 mL), diluted with EtOAc (15 mL), dried over Na₂SO₄, filtered through Celite and concentrated. The crude residue was purified by column chromatography over silica gel using 1% MeOH in DCM to give (2S,3R,4R,5R)-tert-butyl 3-((tert-butyldimethylsilyl)oxy)-5-(2,4-dioxo-3,4-dihydropyrimidin-1(2H)-yl)-4-fluorotetrahydrofuran-2-carboxylate (6.72 g, 83%)

### Example 42. Synthesis of Compound 484

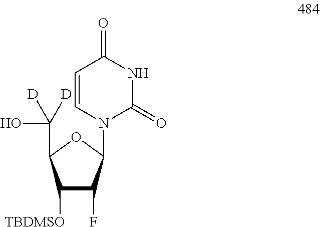

To a solution of (2S,3R,4R,5R)-tert-butyl 3-((tert-butyldimethylsilyl)oxy)-5-(2,4-dioxo-3,4-dihydropyrimidin-1(2H)-yl)-4-fluorotetrahydrofuran-2-carboxylate (3.29 g, 7.64 mmol) was added sodium borodeuteride (1.422 g, 30.6 mmol) in one portion. The reaction was stirred at 80° C. for 20 hours in a sealed tube. The mixture was cooled to room temperature and then quenched with acetic acid (6.99 ml, 122 mmol). The mixture was neutralized with saturated aqueous sodium bicarbonate and extracted with EtOAc. After concentrating, the resulting residue was purified by column chromatography over silica gel (Rf=0.5 hexane EtOAc 1:1) to give [5′-²H₂]-2′-deoxy-2′-fluoro-3′-O-(tert-butyldimethylsilyl)uridine (lg, 36%).

### Example 43. Synthesis of Compound 484

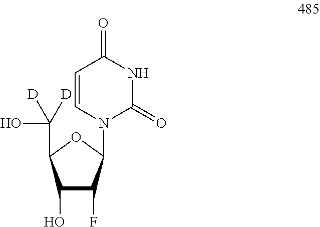

To a solution of [5′-²H₂]-2′-deoxy-2′-fluoro-3′-O-(tert-butyldimethylsilyl)uridine (200 mg, 0.552 mmol) in MeOH (6 mL) was added Dowex 50WX8 (H+ form) (6 g) in one portion. The mixture was stirred for 72 h, filtered and concentrated to give [5′-²H₂]-2′-deoxy-2′-fluorouridine (150 mg, quant.).

### Example 44. Synthesis of Compound 486

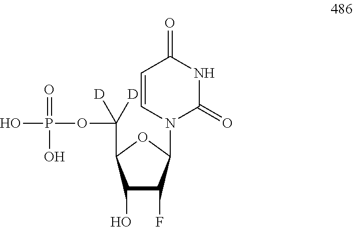

To a solution of phosphoryl trichloride (1.69 mL, 18.13 mmol) in trimethyl phosphate (2 mL) at 5° C., under N2, was added [5′-²H₂]-2′-deoxy-2′-fluorouridine (100 mg, 0.403 mmol) in small portions. The solution was stirred vigorously for 2 h at 5° C. and then was quenched by dropwise addition of DI water (8 mL). The reaction mixture was extracted with chloroform (2×10 mL), and the aqueous phase was treated with concentrated with NH₄OH to pH 6.5, while keeping the solution below 30° C. The aqueous layer was extracted once more with chloroform (10 mL) and then concentrated to dryness. The residue was suspended in MeOH (15 mL), filtered, and concentrated. The resulting solid was purified by column chromatography over silica gel (7:2:1 iPrOH/conc. NH₄OH, H₂O, Rf=0.2). The product was further purified by column chromatography over DEAE using methanol followed by a mobile phase gradient from 0 to 100 mM aqueous ammonium bicarbonate. Fractions were concentrated to dryness, dissolved in water and lyophilized to give [5′-²H₂]-2′-deoxy-2′-fluorouridine-5′-monophosphate (27 mg, 20%) as an amorphous white solid.

### Example 45. Synthesis of Compound 487

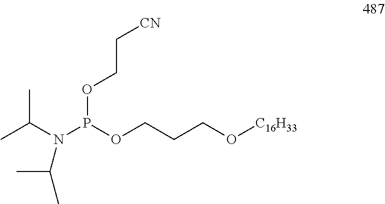

A suspension of 3-hexadecyloxypropan-1-ol (2.02 g, 6.72 mmol) and DIPEA (4.7 mL, 26.9 mmol) in anhydrous methylene chloride (45 mL) was treated dropwise over a 10 minute period with 3-((chloro(diisopropylamino)phosphino)oxy)propanenitrile (3 mL, 13.45 mmol). After 18 hours at room temperature, the mixture was quenched with saturated sodium bicarbonate solution (15 mL) and extracted with ethyl acetate (2×100 mL). Combined organic phases were concentrated to dryness, and the resulting crude residue purified by chromatography over silica gel (25 mm×140 mm) using a solvent gradient from 10 to 20% ethyl acetate in hexanes to give hexadecyloxypropyl-(2-cyanoethyl) diisopropylphosphoramidite (2.1 g, 65%) as a white solid.

¹H NMR (400 MHz, Chloroform-d) δ 3.89-3.54 (m, 6H), 3.49 (t, J=6.3 Hz, 2H), 3.39 (t, J=6.7 Hz, 2H), 2.64 (t, J=6.6 Hz, 2H), 1.87 (p, J=6.3 Hz, 2H), 1.57 (p, J=6.3 Hz, 2H), 1.25 (s, 26H), 1.18 (dd, J=6.8, 3.5 Hz, 12H), 0.87 (t, J=6.6 Hz, 3H).

³¹P NMR (162 MHz, Chloroform-d) δ 147.40.

### Example 46. Synthesis of Compound 488

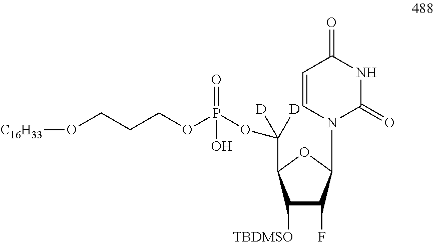

A solution of [5′-²H₂]-2′-deoxy-2-fluoro-3′-O-(tert-butyldimethylsilyl)uridine (600 mg, 1.65 mmol) and hexadecyloxypropyl-(2-cyanoethyl) diisopropylphosphoramidite (1.65 g, 3.31 mmol) in anhydrous THF (22 mL) was treated dropwise with 1-H-tetrazole (14.7 mL of 0.45 M solution in acetonitrile, 6.62 mmol). After 16 hours at room temperature, the mixture was treated dropwise with tert-butyl hydroperoxide (1.5 mL of a 5.5 M solution in nonane, 8.28 mmol) and stirred at room temperature for 1 hour and then quenched with 1.0 M aqueous solution of sodium thiosulfate (40 mL). After 30 min, the mixture was extracted with ethyl acetate (2×80 mL). Combined organic phases were washed with brine (40 mL) and dried over sodium sulfate, filtered, and concentrated. The resulting residue was purified by column chromatography over silica gel (40 g) with a mobile phase gradient from 1% to 5% methanol in methylene chloride to give the cyanoethyl phosphate intermediate which without further purification was dissolved in methanol (30 mL) and treated with concentrated ammonium hydroxide (5 mL, 128 mmol). After 4 hours at room temperature, the mixture was concentrated to dryness. The resulting residue was purified by column chromatography over silica gel using a CombiFlash instrument equipped with a 40 g silica cartridge eluting with a solvent gradient from 5 to 25% methanol in methylene chloride to give [5′-²H₂]-2′-deoxy-2′-fluoro-3′-O-(tert-butyldimethylsilyl)-5′-((hexadecyloxypropyl) phospho)uridine (1 g, 82%) as a white foam.

### Example 47. Synthesis of Compound 489

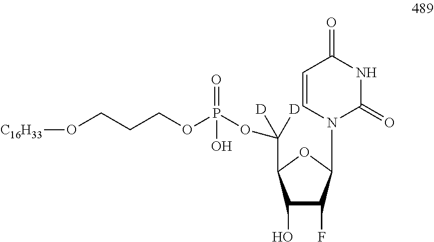

A solution of [5′-²H₂]-2′-deoxy-2′-fluoro-3′-O-(tert-butyldimethylsilyl)-5′-((hexadecyloxypropyl) phospho)uridine (1 g, 1.38 mmol) in THF (15 mL) was treated with acetic acid (0.5 g, 8.28 mmol) and triethylammonium fluoride (1.2 g, 5.52 mmol). After 36 hours, the mixture was concentrated and the resulting residue eluted through a short column (11 mm×90 mm) of Dowex 50WX8 (H⁺ form) using methanol (120 mL) as the mobile phase. The product was further purified by column chromatography over silica gel (24 g) using a mobile phase gradient from 0 to 25% methanol in methylene chloride with 2.5% (v/v) ammonium hydroxide. Pure fractions were pooled and concentrated. The resulting solid was co-evaporated with methylene chloride (2×75 mL) and then dried under high vacuum for 19 hours to give [5′-²H₂]-2′-deoxy-2′-fluoro-5′-((hexadecyloxypropyl)phospho)-uridine (455 mg, 54%) as a white solid.

¹H NMR (400 MHz, Chloroform-d4/Methanol-d₄) δ 7.75 (d, J=8.1 Hz, 1H), 5.95 (dd, J=17.9, 1.6 Hz, 1H), 5.70 (d, J=8.1 Hz, 1H), 5.01 (ddd, J=52.8, 4.6, 1.7 Hz, 1H), 4.30 (ddd, J=20.7, 8.1, 4.5 Hz, 1H), 4.16-4.07 (m, 3H), 3.51 (t, J=6.2 Hz, 2H), 3.41 (t, J=6.7 Hz, 2H), 1.92 (p, J=7.6 Hz, 2H), 1.53 (p, J=7.6 Hz, 2H), 1.25 (s, 26H), 0.87 (d, J=7.6 Hz, 3H).

¹³C NMR (101 MHz, Chloroform-d4/Methanol-d4) δ 164.31, 150.24, 140.33, 102.11, 94.19, 92.32, 88.88, 88.53, 80.83, 80.75, 71.18, 67.62, 67.45, 66.50, 66.40, 64.83, 64.77, 63.81, 31.81, 30.37, 30.29, 29.59, 29.57, 29.54, 29.51, 29.47, 29.41, 29.25, 26.00, 25.96, 22.57, 13.96.

³¹P NMR (162 MHz, Chloroform-d4/Methanol-d4) δ −0.87.

HRMS C₂₈H₄₉D₂FN₂O₉P [M+H⁺]; calculated: 611.34359, found: 611.34363.

### Example 48. Assay Protocols

### (1) Screening Assays for DENV, JEV, POWV, WNV, YFV, PTV, RVFV, CHIKV, EEEV, VEEV, WEEV, TCRV, PCV, JUNV, MPRLV.

Primary cytopathic effect (CPE) reduction assay. Four-concentration CPE inhibition assays are performed. Confluent or near-confluent cell culture monolayers in 96-well disposable microplates are prepared. Cells are maintained in MEM or DMEM supplemented with FBS as required for each cell line. For antiviral assays the same medium is used but with FBS reduced to 2% or less and supplemented with 50 g/ml gentamicin. The test compound is prepared at four log₁₀ final concentrations, usually 0.1, 1.0, 10, and 100 g/ml or M. The virus control and cell control wells are on every microplate. In parallel, a known active drug is tested as a positive control drug using the same method as is applied for test compounds. The positive control is tested with each test run. The assay is set up by first removing growth media from the 96-well plates of cells. Then the test compound is applied in 0.1 ml volume to wells at 2× concentration. Virus, normally at <100 50% cell culture infectious doses (CCID₅₀) in 0.1 ml volume, is placed in those wells designated for virus infection. Medium devoid of virus is placed in toxicity control wells and cell control wells. Virus control wells are treated similarly with virus. Plates are incubated at 37° C. with 5% CO₂ until maximum CPE is observed in virus control wells. The plates are then stained with 0.011% neutral red for approximately two hours at 37° C. in a 5% CO₂ incubator. The neutral red medium is removed by complete aspiration, and the cells may be rinsed 1× with phosphate buffered solution (PBS) to remove residual dye. The PBS is completely removed and the incorporated neutral red is eluted with 50% Sorensen's citrate buffer/50% ethanol (pH 4.2) for at least 30 minutes. Neutral red dye penetrates into living cells, thus, the more intense the red color, the larger the number of viable cells present in the wells. The dye content in each well is quantified using a 96-well spectrophotometer at 540 nm wavelength. The dye content in each set of wells is converted to a percentage of dye present in untreated control wells using a Microsoft Excel computer-based spreadsheet. The 50% effective (EC₅₀, virus-inhibitory) concentrations and 50% cytotoxic (CC₅₀, cell-inhibitory) concentrations are then calculated by linear regression analysis. The quotient of CC₅₀ divided by EC₅₀ gives the selectivity index (SI) value.

Secondary CPE Virus yield reduction (VYR) assay. This assay involves similar methodology to what is described in the previous paragraphs using 96-well microplates of cells. The differences are noted in this section. Eight half-log₁₀ concentrations of inhibitor are tested for antiviral activity and cytotoxicity. After sufficient virus replication ° Ccurs, a sample of supernatant is taken from each infected well (three replicate wells are pooled) and held for the VYR portion of this test, if needed. Alternately, a separate plate may be prepared and the plate may be frozen for the VYR assay. After maximum CPE is observed, the viable plates are stained with neutral red dye. The incorporated dye content is quantified as described above. The data generated from this portion of the test are neutral red EC₅₀, CC₅₀, and SI values. Compounds observed to be active above are further evaluated by VYR assay. The VYR test is a direct determination of how much the test compound inhibits virus replication. Virus that was replicated in the presence of test compound is titrated and compared to virus from untreated, infected controls. Titration of pooled viral samples (collected as described above) is performed by endpoint dilution. This is accomplished by titrating log₁₀ dilutions of virus using 3 or 4 microwells per dilution on fresh monolayers of cells by endpoint dilution. Wells are scored for presence or absence of virus after distinct CPE (measured by neutral red uptake) is observed.

Plotting the log₁₀ of the inhibitor concentration versus log₁₀ of virus produced at each concentration allows calculation of the 90% (one log₁₀) effective concentration by linear regression. Dividing EC₉₀ by the CC₅₀ obtained in part 1 of the assay gives the SI value for this test.

### (2) Screening Assays for Lassa Fever Virus (LASV).

Primary Lassa fever virus assay. Confluent or near-confluent cell culture monolayers in 12-well disposable cell culture plates are prepared. Cells are maintained in DMEM supplemented with 10% FBS. For antiviral assays the same medium is used but with FBS reduced to 2% or less and supplemented with 1% penicillin/streptomycin. The test compound is prepared at four log₁₀ final concentrations, usually 0.1, 1.0, 10, and 100 μg/ml or μM. The virus control and cell control will be run in parallel with each tested compound. Further, a known active drug is tested as a positive control drug using the same experimental set-up as described for the virus and cell control. The positive control is tested with each test run. The assay is set up by first removing growth media from the 12-well plates of cells, and infecting cells with 0.01 MOI of LASV strain Josiah. Cells will be incubated for 90 min: 500 μl inoculum/M12 well, at 37° C., 5% CO2 with constant gentle rocking. The inoculums will be removed and cells will be washed 2× with medium. Then the test compound is applied in 1 ml of total volume of media. Tissue culture supernatant (TCS) will be collected at appropriate time points. TCS will then be used to determine the compounds inhibitory effect on virus replication. Virus that was replicated in the presence of test compound is titrated and compared to virus from untreated, infected controls. For titration of TCS, serial ten-fold dilutions will be prepared and used to infect fresh monolayers of cells. Cells will be overlaid with 1% agarose mixed 1:1 with 2× MEM supplemented with 10% FBS and 1% penecillin, and the number of plaques determined. Plotting the log₁₀ of the inhibitor concentration versus log₁₀ of virus produced at each concentration allows calculation of the 90% (one log₁₀) effective concentration by linear regression.

Secondary Lassa fever virus assay. The secondary assay involves similar methodology to what is described in the previous paragraphs using 12-well plates of cells. The differences are noted in this section. Cells are being infected as described above but this time overlaid with 1% agarose diluted 1:1 with 2×MEM and supplemented with 2% FBS and 1% penicillin/streptomycin and supplemented with the corresponding drug concentration. Cells will be incubated at 37° C. with 5% CO2 for 6 days. The overlay is then removed and plates stained with 0.05% crystal violet in 10% buffered formalin for approximately twenty minutes at room temperature. The plates are then washed, dried and the number of plaques counted. The number of plaques is in each set of compound dilution is converted to a percentage relative to the untreated virus control. The 50% effective (EC50, virus-inhibitory) concentrations are then calculated by linear regression analysis.

### (3) Screening Assays for Ebola Virus (EBOV) and Nipah Virus (NIV).

Primary Ebola Nipah virus assay. Four-concentration plaque reduction assays are performed. Confluent or near-confluent cell culture monolayers in 12-well disposable cell culture plates are prepared. Cells are maintained in DMEM supplemented with 10% FBS. For antiviral assays the same medium is used but with FBS reduced to 2% or less and supplemented with 1% penicillin/streptomycin. The test compound is prepared at four log₁₀ final concentrations, usually 0.1, 1.0, 10, and 100 g/ml or M. The virus control and cell control will be run in parallel with each tested compound. Further, a known active drug is tested as a positive control drug using the same experimental set-up as described for the virus and cell control. The positive control is tested with each test run. The assay is set up by first removing growth media from the 12-well plates of cells. Then the test compound is applied in 0.1 ml volume to wells at 2× concentration. Virus, normally at approximately 200 plaque-forming units in 0.1 ml volume, is placed in those wells designated for virus infection. Medium devoid of virus is placed in toxicity control wells and cell control wells. Virus control wells are treated similarly with virus. Plates are incubated at 37° C. with 5% CO₂ for one hour. Virus-compound inoculums will be removed, cells washed and overlaid with 1.6% tragacanth diluted 1:1 with 2× MEM and supplemented with 2% FBS and 1% penicillin/streptomycin and supplemented with the corresponding drug concentration. Cells will be incubated at 37° C. with 5% CO₂ for 10 days. The overlay is then removed and plates stained with 0.05% crystal violet in 10% buffered formalin for approximately twenty minutes at room temperature. The plates are then washed, dried and the number of plaques counted. The number of plaques is in each set of compound dilution is converted to a percentage relative to the untreated virus control. The 50% effective (EC₅₀, virus-inhibitory) concentrations are then calculated by linear regression analysis.

Secondary Ebola/NIpah virus assay with VYR component. The secondary assay involves similar methodology to what is described in the previous paragraphs using 12-well plates of cells. The differences are noted in this section. Eight half-log₁₀ concentrations of inhibitor are tested for antiviral activity. One positive control drug is tested per batch of compounds evaluated. For this assay, cells are infected with virus. Cells are being infected as described above but this time incubated with DMEM supplemented with 2% FBS and 1% penicillin/streptomycin and supplemented with the corresponding drug concentration. Cells will be incubated for 10 days at 37° C. with 5% CO₂, daily observed under microscope for the number of green fluorescent cells. Aliquots of supernatant from infected cells will be taken daily and the three replicate wells are pooled. The pooled supernatants are then used to determine the compounds inhibitory effect on virus replication. Virus that was replicated in the presence of test compound is titrated and compared to virus from untreated, infected controls. For titration of pooled viral samples, serial ten-fold dilutions will be prepared and used to infect fresh monolayers of cells. Cells are overlaid with tragacanth and the number of plaques determined. Plotting the log₁₀ of the inhibitor concentration versus log₁₀ of virus produced at each concentration allows calculation of the 90% (one log₁₀) effective concentration by linear regression.

### Example 49. Anti-Dengue Virus Cytoprotection Assay

Cell Preparation—BHK21 cells (Syrian golden hamster kidney cells, ATCC catalog #CCL-I 0), Vero cells (African green monkey kidney cells, ATCC catalog #CCL-81), or Huh-7 cells (human hepatocyte carcinoma) were passaged in DMEM supplemented with 10% FBS, 2 mM L-glutamine, 100 U/mL penicillin, and 100 μg/mL streptomycin in T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 3×10³ (5×10⁵ for Vero cells and Huh-7 cells) cells per well in tissue culture medium and added to flat bottom microtiter plates in a volume of 100 μL. The plates were incubated at 37° C./5% CO₂ overnight to allow for cell adherence. Monolayers were observed to be approximately 70% confluent.

Virus Preparation—The Dengue virus type 2 New Guinea C strain was obtained from ATCC (catalog #VR-1584) and was grown in LLC-MK2 (Rhesus monkey kidney cells; catalog #CCL-7.1) cells for the production of stock virus pools. An aliquot of virus pretitered in BHK21 cells was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, and 100 μg/mL streptomycin) such that the amount of virus added to each well in a volume of 100 μL was the amount determined to yield 85 to 95% cell killing at 6 days post-infection.

Plate Format—Each plate contains cell control wells (cells only), virus control wells (cells plus virus), triplicate drug toxicity wells per compound (cells plus drug only), as well as triplicate experimental wells (drug plus cells plus virus).

Efficacy and Toxicity XTT—Following incubation at 37° C. in a 5% CO₂ incubator, the test plates were stained with the tetrazolium dye XTT (2,3-bis(2-methoxy-4-nitro-5-sulfophenyl)-5-[(phenylamino)carbonyl]-2H-tetrazolium hydroxide). XTT-tetrazolium was metabolized by the mitochondrial enzymes of metabolically active cells to a soluble formazan product, allowing rapid quantitative analysis of the inhibition of virus-induced cell killing by antiviral test substances. XTT solution was prepared daily as a stock of 1 mg/mL in RPMI 1640. Phenazine methosulfate (PMS) solution was prepared at 0.15 mg/mL in PBS and stored in the dark at−20° C. XTT/PMS stock was prepared immediately before use by adding 40 μL of PMS per ml of XTT solution. Fifty microliters of XTT/PMS was added to each well of the plate and the plate was reincubated for 4 hours at 37° C. Plates were sealed with adhesive plate sealers and shaken gently or inverted several times to mix the soluble formazan product and the plate was read spectrophotometrically at 450/650 nm with a Molecular Devices Vmax plate reader.

Data Analysis—Raw data was collected from the Softmax Pro 4.6 software and imported into a Microsoft Excel spreadsheet for analysis. The percent reduction in viral cytopathic effect compared to the untreated virus controls was calculated for each compound. The percent cell control value was calculated for each compound comparing the drug treated uninfected cells to the uninfected cells in medium alone.

### Example 50. Anti-RSV Cytoprotection Assay

Cell Preparation—HEp2 cells (human epithelial cells, A TCC catalog #CCL-23) were passaged in DMEM supplemented with 10% FBS, 2 mM L-glutamine, 100 U/mL penicillin, 100 μg/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 μL. The plates were incubated at 37° C./5% C0₂ overnight to allow for cell adherence. Virus Preparation—The RSV strain Long and RSV strain 9320 were obtained from ATCC (catalog #VR-26 and catalog #VR-955, respectively) and were grown in HEp2 cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, 100 μg/mL streptomycin, 1 mM sodium pyruvate, and 0.1 mM NEAA) such that the amount of virus added to each well in a volume of 100 μL was the amount determined to yield 85 to 95% cell killing at 6 days post-infection. Efficacy and Toxicity XTT-Plates were stained and analyzed as previously described for the Dengue cytoprotection assay.

### Example 51. Anti-Influenza Virus Cytoprotection Assay

Cell Preparation-MOCK cells (canine kidney cells, ATCC catalog #CCL-34) were passaged in DMEM supplemented with 10% FBS, 2 mM L-glutamine, 100 U/mL penicillin, 100 μg/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 μL. The plates were incubated at 37° C./5% C0₂ overnight to allow for cell adherence.

Virus Preparation—The influenza A/PR/8/34 (A TCC #VR-95), A/CA/05/09 (CDC), A/NY/18/09 (CDC) and A/NWS/33 (ATCC #VR-219) strains were obtained from ATCC or from the Center of Disease Control and were grown in MDCK cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 0.5% BSA, 2 mM L-glutamine, 100 U/mL penicillin, 100 μg/mL streptomycin, 1 mM sodium pyruvate, 0.1 mM NEAA, and 1 μg/ml TPCK-treated trypsin) such that the amount of virus added to each well in a volume of 100 μL was the amount determined to yield 85 to 95% cell killing at 4 days post-infection. Efficacy and Toxicity XTT-Plates were stained and analyzed as previously described for the Dengue cytoprotection assay.

### Example 52. Anti-Hepatitis C Virus Assay

Cell Culture—The reporter cell line Huh-luc/neo-ET was obtained from Dr. Ralf Bartenschlager (Department of Molecular Virology, Hygiene Institute, University of Heidelberg, Germany) by ImQuest BioSciences through a specific licensing agreement. This cell line harbors the persistently replicating I₃₈₉luc-ubi-neo/NS3-3′/ET replicon containing the firefly luciferase gene-ubiquitin-neomycin phosphotransferase fusion protein and EMCV IRES driven NS3-5B HCV coding sequences containing the ET tissue culture adaptive mutations (E1202G, T12081, and K1846T). A stock culture of the Huh-luc/neo-ET was expanded by culture in DMEM supplemented with I 0% FCS, 2 mM glutamine, penicillin (100 μU/mL)/streptomycin (100 μg/mL) and I× nonessential amino acids plus 1 mg/mL G418. The cells were split 1:4 and cultured for two passages in the same media plus 250 μg/mL G418. The cells were treated with trypsin and enumerated by staining with trypan blue and seeded into 96-well tissue culture plates at a cell culture density 7.5×10³ cells per well and incubated at 37° C. 5% C0₂ for 24 hours. Following the 24 hour incubation, media was removed and replaced with the same media minus the G418 plus the test compounds in triplicate. Six wells in each plate received media alone as a no-treatment control. The cells were incubated an additional 72 hours at 37° C. 5% C0₂ then anti-HCV activity was measured by luciferase endpoint. Duplicate plates were treated and incubated in parallel for assessment of cellular toxicity by XTT staining.

Cellular Viability—The cell culture monolayers from treated cells were stained with the tetrazolium dye XTT to evaluate the cellular viability of the Huh-luc/neo-ET reporter cell line in the presence of the compounds.

Measurement of Virus Replication—HCV replication from the replicon assay system was measured by luciferase activity using the britelite plus luminescence reporter gene kit according to the manufacturer's instructions (Perkin Elmer, Shelton, CT). Briefly, one vial of britelite plus lyophilized substrate was solubilized in 10 mL of britelite reconstitution buffer and mixed gently by inversion. After a 5 minute incubation at room temperature, the britelite plus reagent was added to the 96 well plates at 100 μL per well. The plates were sealed with adhesive film and incubated at room temperature for approximately 10 minutes to lyse the cells. The well contents were transferred to a white 96-well plate and luminescence was measured within 15 minutes using the Wallac 1450 Microbeta Trilux liquid scintillation counter. The data were imported into a customized Microsoft Excel 2007 spreadsheet for determination of the 50% virus inhibition concentration (ECSO).

### Example 53. Anti-Parainfluenza-3 Cytoprotection Assay

Cell Preparation—HEp2 cells (human epithelial cells, ATCC catalog #CCL-23) were passaged in DMEM supplemented with 10% FBS, 2 mM L-glutamine, 100 U/mL penicillin, 100 μg/mL streptomycin 1 mM sodium pyruvate, and 0.1 mM NEAA, T-75 flasks prior to use in the antiviral assay. On the day preceding the assay, the cells were split 1:2 to assure they were in an exponential growth phase at the time of infection. Total cell and viability quantification was performed using a hemocytometer and Trypan Blue dye exclusion. Cell viability was greater than 95% for the cells to be utilized in the assay. The cells were resuspended at 1×10⁴ cells per well in tissue culture medium and added to flat bottom microtiter plates in a volume of 100 μL. The plates were incubated at 37° C./5% C0₂ overnight to allow for cell adherence.

Virus Preparation—The Parainfluenza virus type 3 SF4 strain was obtained from ATCC (catalog #VR-281) and was grown in HEp2 cells for the production of stock virus pools. A pretitered aliquot of virus was removed from the freezer (−80° C.) and allowed to thaw slowly to room temperature in a biological safety cabinet. Virus was resuspended and diluted into assay medium (DMEM supplemented with 2% heat-inactivated FBS, 2 mM L-glutamine, 100 U/mL penicillin, and 100 μg/mL streptomycin) such that the amount of virus added to each well in a volume of 100 μL was the amount determined to yield 85 to 95% cell killing at 6 days post-infection.

Plate Format—Each plate contains cell control wells (cells only), virus control wells (cells plus virus), triplicate drug toxicity wells per compound (cells plus drug only), as well a triplicate experimental wells (drug plus cells plus virus). Efficacy and Toxicity XTT—Following incubation at 37° C. in a 5% C0² incubator, the test plates were stained with the tetrazolium dye XTT (2,3-bis(2-methoxy-4-nitro-5-sulfophenyl)-5-[(phenylamino)carbonyl]-2H-tetrazol hydroxide). XTT-tetrazolium was metabolized by the mitochondrial enzymes of metabolically active cells to a soluble formazan product, allowing rapid quantitative analysis of the inhibition of virus-induced cell killing by antiviral test substances. XTT solution was prepared daily as a stock of 1 mg/mL in RPMI1640. Phenazine methosulfate (PMS) solution was prepared at 0.15 mg/mL in PBS and stored in the dark at −20° C. XTT/PMS stock was prepared immediately before use by adding 40 μL of PMS per ml of XTT solution. Fifty microliters of XTT/PMS was added to each well of the plate and the plate was reincubated for 4 hours at 37° C. Plates were sealed with adhesive plate sealers and shaken gently or inverted several times to mix the soluble fomlazan product and the plate was read spectrophotometrically at 450/650 nm with a Molecular Devices Vmax plate reader.

Data Analysis—Raw data was collected from the Softmax Pro 4.6 software and imported into a Microsoft Excel spreadsheet for analysis. The percent reduction in viral cytopathic effect compared to the untreated virus controls was calculated for each compound. The percent cell control value was calculated for each compound comparing the drug treated uninfected cells to the uninfected cells in medium alone.

### Example 54. Influenza Polymerase Inhibition Assay

Virus Preparation—Purified influenza virus A/PR/8/34 (1 ml) was obtained from Advanced Biotechnologies, Inc. (Columbia, MD), thawed and dispensed into five aliquots for storage at −80° C. until use. On the day of assay set up, 20 μL of 2.5% Triton N-101 was added to 180 μL of purified virus. The disrupted virus was diluted 1:2 in a solution containing 0.25% Triton and PBS. Disruption provided the source of influenza ribonucleoprotein (RNP) containing the influenza RNA-dependent RNA polymerase and template RNA. Samples were stored on ice until use in the assay.

Polymerase reaction—Each 50 μL polymerase reaction contained the following: 5 μL of the disrupted RNP, 100 mM Tris-HCl (pH 8.0), 100 mM KCl, 5 mM MgCl₂. 1 mM dithiothreitol, 0.25% Triton N-101, 5 μCi of [α-³²P]GTP, 100 μM ATP, 50 μM each (CTP, UTP), 1 μM GTP, and 200 μM adenyl (3′-5′) guanosine. For testing the inhibitor, the reactions contained the inhibitor and the same was done for reactions containing the positive control (2′-Deoxy-2′-fluoroguanosine-5′-triphosphate). Other controls included RNP+reaction mixture, and RNP+I % DMSO. The reaction mixture without the ApG primer and NTPs was incubated at 30° C. for 20 minutes. Once the ApG and NTPs were added to the reaction mixture, the samples were incubated at 30° C. for 1 hour then immediately followed by the transfer of the reaction onto glass-fiber filter plates and subsequent precipitation with 10% trichloroacetic acid (TCA). The plate was then washed five times with 5% TCA followed by one wash with 95% ethanol. Once the filter had dried, incorporation of [α-³²P]GTP was measured using a liquid scintillation counter (Micro beta).

Plate Format—Each test plate contained triplicate samples of the three compounds (6 concentrations) in addition to triplicate samples of RNP+reaction mixture (RNP alone), RNP+1% DMSO, and reaction mixture alone (no RNP).

Data Analysis—Raw data was collected from the Micro Beta scintillation counter. The incorporation of radioactive GTP directly correlates with the levels of polymerase activity. The “percent inhibition values” were obtained by dividing the mean value of each test compound by the RNP+1% DMSO control. The mean obtained at each concentration of 2DFGTP was compared to the RNP+reaction control. The data was then imported into Microsoft Excel spreadsheet to calculate the IC₅₀ values by linear regression analysis.

### Example 55. HCV Polymerase Inhibition Assay

Activity of compounds for inhibition of HCV polymerase was evaluated using methods previously described (Lam eta!. 2010. Antimicrobial Agents and Chemotherapy 54(8):3187-3196). HCV NS5B polymerase assays were performed in 20 μL volumes in 96 well reaction plates. Each reaction contained 40 ng/μL purified recombinant NS5BΔ22 genotype-1b polymerase, 20 ng/μL of HCV genotype-1b complimentary IRES template, 1 μM of each of the four natural ribonucleotides, 1 U/mL Optizyme RNAse inhibitor (Promega, Madison, WI), 1 mM MgCl₂, 0.75 mM MnCl₂, and 2 mM dithiothreitol (DTT) in 50 mM HEPES buffer (pH 7.5). Reaction mixtures were assembled on ice in two steps. Step 1 consisted of combining all reaction components except the natural nucleotides and labeled UTP in a polymerase reaction mixture. Ten microliters (10 μL) of the polymerase mixture was dispensed into individual wells of the 96 well reaction plate on ice. Polymerase reaction mixtures without NS5B polymerase were included as no enzyme controls. Serial half-logarithmic dilutions of test and control compounds, 2′-O-Methyl-CTP and 2′-O-Methyl-GTP (Trilink, San Diego, CA), were prepared in water and 5 μL of the serial diluted compounds or water alone (no compound control) were added to the wells containing the polymerase mixture. Five microliters of nucleotide mix (natural nucleotides and labeled UTP) was then added to the reaction plate wells and the plate was incubated at 27° C. for 30 minutes. The reactions were quenched with the addition of 80 μL stop solution (12.5 mM EDTA, 2.25 M NaCl, and 225 mM sodium citrate) and the RNA products were applied to a Hybond-N+ membrane (GE Healthcare, Piscataway, N.J) under vacuum pressure using a dot blot apparatus. The membrane was removed from the dot blot apparatus and washed four times with 4×SSC (0.6 M NaCl, and 60 mM sodium citrate), and then rinsed one time with water and once with 100% ethanol. The membrane was air dried and exposed to a phosphoimaging screen and the image captured using a Typhoon 8600 Phospho imager. Following capture of the image, the membrane was placed into a Micro beta cassette along with scintillation fluid and the CPM in each reaction was counted on a Micro beta 1450. CPM data were imported into a custom Excel spreadsheet for determination of compound IC₅₀s.

### Example 56. NS5B RNA-Dependent RNA Polymerase Reaction Conditions

Compounds were assayed for inhibition of NS5B-δ21 from HCV GT-1b Con-1.

Reactions included purified recombinant enzyme, 1 u/μL negative-strand HCV IRES RNA template, and 1 μM NTP substrates including either [³²P]-CTP or [³²P]-UTP. Assay plates were incubated at 27° C. for 1 hour before quench. [³²P] incorporation into macromolecular product was assessed by filter binding.

### Example 57. Human DNA Polymerase Inhibition Assay

The human DNA polymerase alpha (catalog #1075), beta (catalog #1077), and gamma (catalog #1076) were purchased from CHIMERx (Madison, WI). Inhibition of beta and gamma DNA polymerase activity was assayed in microtiter plates in a 50 μL reaction mixture containing 50 mM Tris-HCl (pH 8.7), KCl (10 mM for beta and 100 mM for gamma), 10 mM MgCl₂, 0.4 mg/mL BSA, 1 mM DTT, 15% glycerol, 0.05 mM of dCTP, dTTP, and dATP, 10 μCi [³²P]-alpha-dGTP (800 Ci/mmol), 20 ug activated calf thymus DNA and the test compound at indicated concentrations. The alpha DNA polymerase reaction mixture was as follows in a 50 μL volume per sample: 20 mM Tris-HCl (pH 8), 5 mM magnesium acetate, 0.3 mg/mL BSA, 1 mM DTT, 0.1 mM spermine, 0.05 mM of dCTP, dTTP, and dATP, 10 μCi [³²P]-alpha-dGTP (800 Ci/mmol), 20 ug activated calf thymus DNA and the test compound at the indicated concentrations. For each assay, the enzyme reactions were allowed to proceed for 30 minutes at 37° C. followed by the transfer onto glass-fiber filter plates and subsequent precipitation with 10% trichloroacetic acid (TCA). The plate was then washed with 5% TCA followed by one wash with 95% ethanol. Once the filter had dried, incorporation of radioactivity was measured using a liquid scintillation counter (Microbeta).

### Example 58. HIV Infected PBMC Assay

Fresh human peripheral blood mononuclear cells (PBMCs) were obtained from a commercial source (Biological Specialty) and were determined to be seronegative for HIV and HBV. Depending on the volume of donor blood received, the leukophoresed blood cells were washed several times with PBS. After washing, the leukophoresed blood was diluted 1:1 with Dulbecco's phosphate buffered saline (PBS) and layered over 15 mL of Ficoll-Hypaque density gradient in a 50 ml conical centrifuge tube. These tubes were centrifuged for 30 min at 600 g. Banded PBMCs were gently aspirated from the resulting interface and washed three times with PBS. After the final wash, cell number was determined by Trypan Blue dye exclusion and cells were re-suspended at 1×10\hat{ }6 cells/mL in RPMI 1640 with 15% Fetal Bovine Serum (FBS), 2 mmol/L L-glutamine, 2 μg/mL PHA-P, 100 U/mL penicillin and 100 μg/mL streptomycin and allowed to incubate for 48-72 hours at 37° C. After incubation, PBMCs were centrifuged and resuspended in tissue culture medium. The cultures were maintained until use by half-volume culture changes with fresh IL-2 containing tissue culture medium every 3 days. Assays were initiated with PBMCs at 72 hours post PHA-P stimulation.

To minimize effects due to donor variability, PBMCs employed in the assay were a mixture of cells derived from 3 donors. Immediately prior to use, target cells were resuspended in fresh tissue culture medium at 1×10\hat{ }6 cells/mL and plated in the interior wells of a 96-well round bottom microtiter plate at 50 μL/well. Then, 100 μL of 2× concentrations of compound-containing medium was transferred to the 96-well plate containing cells in 50 μL of the medium. AZT was employed as an internal assay standard.

Following addition of test compound to the wells, 50 μL of a predetermined dilution of HIV virus (prepared from 4× of final desired in-well concentration) was added, and mixed well. For infection, 50-150 TCID₅₀ of each virus was added per well (final MOI approximately 0.002). PBMCs were exposed in triplicate to virus and cultured in the presence or absence of the test material at varying concentrations as described above in the 96-well microtiter plates. After 7 days in culture, HIV-1 replication was quantified in the tissue culture supernatant by measurement of reverse transcriptase (RT) activity. Wells with cells and virus only served as virus controls. Separate plates were identically prepared without virus for drug cytotoxicity studies.

Reverse Transcriptase Activity Assay—Reverse transcriptase activity was measured in cell-free supernatants using a standard radioactive incorporation polymerization assay. Tritiated thymidine triphosphate (TTP; New England Nuclear) was purchased at 1 Ci/mL and 1 μL was used per enzyme reaction. A rAdT stock solution was prepared by mixing 0.5 mg/mL poly rA and 1.7 U/mL oligo dT in distilled water and was stored at −20° C. The RT reaction buffer was prepared fresh daily and consists of 125 μL of 1 mol/L EGTA, 125 μL of dH₂O, 125 μL of 20% Triton X-100, 50 μL of 1 mol/L Tris (pH 7.4), 50 μL of 1 mol/L DTT, and 40 μL of 1 mol/L MgCl₂. For each reaction, 1 μL of TTP, 4 μL of dH₂O, 2.5 μL of rAdT, and 2.5 μL of reaction buffer were mixed. Ten microliters of this reaction mixture was placed in a round bottom microtiter plate and 15 μL of virus-containing supernatant was added and mixed. The plate was incubated at 37° C. in a humidified incubator for 90 minutes. Following incubation, 10 μL of the reaction volume was spotted onto a DEAE filter mat in the appropriate plate format, washed 5 times (5 minutes each) in a 5% sodium phosphate buffer, 2 times (1 minute each) in distilled water, 2 times (1 minute each) in 70% ethanol, and then air dried. The dried filtermat was placed in a plastic sleeve and 4 mL of Opti-Fluor O was added to the sleeve. Incorporated radioactivity was quantified utilizing a Wallac 1450 Microbeta Trilux liquid scintillation counter.

### Example 59. HBV Assay

HepG2.2.15 cells (100 μL) in RPMI1640 medium with 10% fetal bovine serum was added to all wells of a 96-well plate at a density of 1×10⁴ cells per well and the plate was incubated at 37° C. in an environment of 5% CO₂ for 24 hours. Following incubation, six ten-fold serial dilutions of test compound prepared in RPMI1640 medium with 10% fetal bovine serum were added to individual wells of the plate in triplicate. Six wells in the plate received medium alone as a virus only control. The plate was incubated for 6 days at 37° C. in an environment of 5% CO₂. The culture medium was changed on day 3 with medium containing the indicated concentration of each compound. One hundred microliters of supernatant was collected from each well for analysis of viral DNA by qPCR and cytotoxicity was evaluated by XTT staining of the cell culture monolayer on the sixth day.

Ten microliters of cell culture supernatant collected on the sixth day was diluted in qPCR dilution buffer (40 μg/mL sheared salmon sperm DNA) and boiled for 15 minutes. Quantitative real time PCR was performed in 386 well plates using an Applied Biosystems 7900HT Sequence Detection System and the supporting SDS 2.4 software. Five microliters (5 μL) of boiled DNA for each sample and serial 10-fold dilutions of a quantitative DNA standard were subjected to real time Q-PCR using Platinum Quantitative PCR SuperMix-UDG (Invitrogen) and specific DNA oligonucleotide primers (IDT, Coralville, ID) HBV-AD38-qF1 (5′-CCG TCT GTG CCT TCT CAT CTG-3′), HBV-AD38-qR1 (5′-AGT CCA AGA GTY CTC TTA TRY AAG ACC TT-3′), and HBV-AD38-qP1 (5′-FAM CCG TGT GCA/ZEN/CTT CGC TTC ACC TCT GC-3′BHQ1) at a final concentration of 0.2 μM for each primer in a total reaction volume of 15 μL. The HBV DNA copy number in each sample was interpolated from the standard curve by the SDS.24 software and the data were imported into an Excel spreadsheet for analysis.

The 50% cytotoxic concentration for the test materials are derived by measuring the reduction of the tetrazolium dye XTT in the treated tissue culture plates. XTT is metabolized by the mitochondrial enzyme NADPH oxidase to a soluble formazan product in metabolically active cells. XTT solution was prepared daily as a stock of 1 mg/mL in PBS. Phenazine methosulfate (PMS) stock solution was prepared at 0.15 mg/mL in PBS and stored in the dark at −20° C. XTT/PMS solution was prepared immediately before use by adding 40 μL of PMS per 1 mL of XTT solution. Fifty microliters of XTT/PMS was added to each well of the plate and the plate incubated for 2-4 hours at 37° C. The 2-4 hour incubation has been empirically determined to be within linear response range for XTT dye reduction with the indicated numbers of cells for each assay. Adhesive plate sealers were used in place of the lids, the sealed plate was inverted several times to mix the soluble formazan product and the plate was read at 450 nm (650 nm reference wavelength) with a Molecular Devices SpectraMax Plus 384 spectrophotometer. Data were collected by Softmax 4.6 software and imported into an Excel spreadsheet for analysis.

### Example 60. Dengue RNA-Dependent RNA Polymerase Reaction Conditions

RNA polymerase assay was performed at 30° C. using 100 μl reaction mix in 1.5 ml tube. Final reaction conditions were 50 mM Hepes (pH 7.0), 2 mM DTT, 1 mM MnCl₂, 10 mM KCl, 100 nM UTR-Poly A (self-annealing primer), 10 μM UTP, 26 nM RdRp enzyme. The reaction mix with different compounds (inhibitors) was incubated at 30° C. for 1 hour. To assess amount of pyrophosphate generated during polymerase reaction, 30 μl of polymerase reaction mix was mixed with a luciferase coupled-enzyme reaction mix (70 μl). Final reaction conditions of luciferase reaction were 5 mM MgCl₂, 50 mM Tris-HCl (pH 7.5), 150 mM NaCl, 200 μU ATP sulfurylase, 5 μM APS, 10 nM Luciferase, 100 μM D-luciferin. White plates containing the reaction samples (100 μl) were immediately transferred to the luminometer Veritas (Turner Biosystems, CA) for detection of the light signal.

### Example 61. Procedure for Cell Incubation and Analysis

Huh-7 cells were seeded at 0.5×10\hat{ }6 cells/well in 1 mL of complete media in 12 well tissue culture treated plates. The cells were allowed to adhere overnight at 370/5% CO₂. A 40 M stock solution of test article was prepared in 100% DMSO. From the 40 M stock solution, a 20 M solution of test article in 25 ml of complete DMEM media was prepared. For compound treatment, the media was aspirated from the wells and 1 mL of the 20 M solution was added in complete DMEM media to the appropriate wells. A separate plate of cells with “no” addition of the compound was also prepared. The plates were incubated at 37°/5% CO₂ for the following time points: 1, 3, 6 and 24 hours. After incubation at the desired time points, the cells were washed 2× with 1 mL of DPBS. The cells were extracted by adding 500 μl of 70% methanol/30% water spiked with the internal standard to each well treated with test article. The non-treated blank plate was extracted with 500 ul of 70% methanol/30% water per well. Samples were centrifuged at 16,000 rpm for 10 minutes at 4° C. Samples were analyzed by LC-MS/MS using an ABSCIEX 5500 QTRAP LC-MS/MS system with a Hypercarb (PGC) column.

### Example 62. Zika RNA-Dependent RNA Polymerase Reaction Conditions

RNA polymerase assay was performed at 30° C. using 100 μl reaction mix in 1.5 ml tube. Final reaction conditions were 50 mM Hepes (pH 7.0), 2 mM DTT, 1 mM MnCl₂, 10 mM KCl, 100 nM UTR-Poly A (self-annealing primer), 10 μM UTP, 26 nM RdRp enzyme. The reaction mix with different compounds (inhibitors) was incubated at 30° C. for 1 hour. To assess amount of pyrophosphate generated during polymerase reaction, 30 μl of polymerase reaction mix was mixed with a luciferase coupled-enzyme reaction mix (70 μl). Final reaction conditions of luciferase reaction were 5 mM MgCl₂, 50 mM Tris-HCl (pH 7.5), 150 mM NaCl, 200 μU ATP sulfurylase, 5 μM APS, 10 nM Luciferase, 100 μM D-luciferin. White plates containing the reaction samples (100 μl) were immediately transferred to the luminometer Veritas (Turner Biosystems, CA) for detection of the light signal.

### Example 63. Zika Infectious Assay Conditions

Vero cells were passaged in DMEM medium in T-75 flasks prior to use in the antiviral assay. On the day preceding the assay, the cells were split 1:2 to assure they were in exponential growth phase at the time of infection. The cells were resuspended at 5×10³ cells per well in tissue culture medium and added to flat bottom microtiter plates in a volume of 100 mL. The plates were incubated at 37° C./5% CO₂ overnight to allow for cell adherence. Separately, Zika virus was titrated in LLCMK2 cells to define the inoculum for use in the antiviral assay. Virus was diluted in DMEM medium such that the amount of virus added to each well in a volume of 100 mL was the amount determined to achieve 85 to 95% cell killing at 5 days post-infection. Following incubation test plates were stained with XTT dye. XTT solution was prepared daily as a stock solution of 1 mg/mL in RPMI1640. PMS solution was prepared at 0.15 mg/mL in PBS and stored in the dark at −20° C. XTT/PMS stock was prepared immediately before use by adding 40 mL of PMS per mL of XTT solution. Fifty microliters of XTT/PMS was added to each well of the plate, and the plate was reincubated for 4 hours at 37° C. Plates were sealed with adhesive plate sealers ad shaken gently to mix the soluble formazan product, and the plate was read spectrophotometrically read 450/650 nm with a Molecular Devices Vmax plate reader. The raw data was collected from Softmax Pro and imported into a Microsoft Excel XLfit4 spreadsheet for analysis using four parameter curve fit calculations.

### Example 64. POLRMT Methods

POLRMT enzyme purification: A variant of human POLRMT coding sequence was amplified from a POLRMT cDNA plasmid (Accession:BC098387, Clone ID: 5264127, Dharmacon, CO) and cloned into a pMal-c5X vector under control of the tac promoter. For protein expression, the plasmid was transformed into Stellar competent cells (Clontech). Expression vector pMal-c5X contains a lac gene which allows inducible expression of POLRMT in Stellar cells. The transformed cells were grown in LB medium containing 100 μg/ml ampicillin at 35° C. to an optical density of 1 at 600 nm. Cells were cooled down in a 4° C. fridge for 1 hour. MgCl₂ was added to final concentration of 1 mM. Protein expression was induced at 16° C. overnight by the addition of 0.4 mM IPTG. Cells were harvested by centrifugation at 4000×g for 20 min at 4° C. The cell pellet was stored at −80° C. until further processed. For protein purification, the cell pellet was re-suspended in sonication buffer (20 mM Tris-HCl pH 7.5, 10% glycerol, 500 mM NaCl, 0.5% Triton X-100, 10 mM DTT, 10 mM MgCl₂, 30 mM imidazole and 1× protease inhibitor cocktail). Cell disruption was performed on ice for 10 min using an ultrasound probe sonicator. The cell extract was clarified by centrifugation at 16,000×g for 20 min at 4° C. The supernatant was incubated with HisPur Ni-NTA agarose resin with gentle rocking for 15 minutes at 4° C. The resin was then washed 5 times with 10 volumes of wash buffer (20 mM Tris-HCl pH 7.5, 10% glycerol, 500 mM NaCl, 0.1% Triton X-100, 1 mM DTT, 2 mM MgCl₂) containing 30 mM imidazole and then once with the wash buffer containing 2M NaCl. The protein was eluted from the resin with 1 volume of elution buffer (20 mM Tris-HCl, pH 7.5, 10% glycerol, 50 mM NaCl, 0.5% Triton X-100, 10 mM DTT and 300 mM imidazole). The eluted enzyme was adjusted to 50% glycerol and stored at −80° C. before use. Protein identification was performed by mass spectrometry. The concentration of a targeted protein was measured by SDS-PAGE using BSA (Sigma, St. Louis, MO) as a standard.

Measurement of ribonucleotide analog incorporation efficiency:Different templates were designed to test individual analog rNTPs, Table 1. Different concentrations of tested ribonucleotide analogs were added to reaction mixtures containing 10 nM P/T and 20 nM POLRMT in a reaction buffer (5 mM Tris-HCl, pH 7.5, 10 mM DTT, 20 mM MgCl₂, 0.5% X-100, 10% glycerol) to initiate the reactions. The reactions were continued at 22° C. for different time and subsequently quenched with quenching buffer (8 M Urea, 90 mM Tris base, 29 mM taurine, 10 mM EDTA, 0.02% SDS and 0.1% bromophenol blue). The quenched samples were denatured at 95° C. for 15 min and the primer extension products were separated using 20% denaturing polyacrylamide gel electrophoresis (Urea PAGE) in 1×TTE buffer (90 mM Tris base, 29 mM Taurine and 0.5 mM EDTA). After electrophoresis, gels were scanned using an Odyssey infrared imaging system. The intensity of different RNA bands was quantified using Image Studio Software Lite version 4.0. The incorporation efficiencies of different rNTP analogs were evaluated by measurement the K_{1/2} and corresponding Discrimination Values (ref. G Lu).

Primer extension polymerase activity assay: POLRMTs polymerase activity was determined in a primer extension reaction using a fluorescently labeled RNA primer/DNA template complex. A typical primer extension reaction was performed in a 20-μl reaction mixture containing reaction buffer (5 mM Tris-HCl, pH7.5, 10 mM DTT, 20 mM MgCl₂, 0.1% Triton X-100, 0.01 U RNasin, 10% glycerol), 10 nM P/T complex, and 20 nM POLRMT. The reaction was initiated by the addition of rNTPs at a final concentration of 100 μM, followed by incubation for 1 h at 22° C. The reactions were quenched by the addition of 20 μl quenching buffer (8 M Urea, 90 mM Tris base, 29 mM taurine, 10 mM EDTA, 0.02% SDS and 0.1% bromophenol blue). The quenched samples were denatured at 95° C. for 15 min and the primer extension products were separated using 20% denaturing polyacrylamide gel electrophoresis (Urea PAGE) in 1×TTE buffer (90 mM Tris base, 29 mM Taurine and 0.5 mM EDTA). After electrophoresis, gels were scanned using an Odyssey infrared imaging system (LI-COR Biosciences, Lincoln, NE). The images were analyzed and the proper RNA bands were quantified using Image Studio software Lite version 4.0 (LI-COR Biosciences, Lincoln, NE).

### Example 65. EIDD-2838 Togaviridae Activity

| | | EC₅₀ | EC₉₀ | CC₅₀ |
|---|---|---|---|---|
| Virus | Cell Line | (μM) | (μM) | (μM) |
| VEEV | Vero76 | 8.28 | | >230 |
| VEEV | Vero76 | 8.51 | | >230 |
| EEEV | Vero76 | 2.76 | | >230 |
| EEEV | Vero76 | 3.22 | | >230 |
| EEEV | Vero 76 | | 28.4 | >230 |
| EEEV | Vero 76 | 12.4 | | >230 |
| WEEV | Vero76 | 5.98 | | >230 |
| WEEV | Vero76 | 7.82 | | >230 |
| WEEV | Vero 76 | | 24.15 | >230 |
| WEEV | Vero 76 | 5.75 | | >230 |
| CHIKV | Vero76 | 2.3 | | >230 |
| CHIKV | Vero76 | 3.68 | | >230 |
| CHIKV | Vero 76 | | 23.92 | >230 |
| CHIKV | Vero 76 | 3.91 | | >230 |

### Example 66. EIDD-2838 Bunyaviridae Activity

| | | EC₅₀ | EC₉₀ | CC₅₀ |
|---|---|---|---|---|
| Virus | Cell Line | (μM) | (μM) | (μM) |
| RVFV | Vero76 | 3.91 | | >230 |
| RVFV | Vero76 | 3.68 | | >230 |
| RVFV | Vero 76 | | 2.19 | >230 |
| RVFV | Vero 76 | 4.14 | | >230 |
| Heartland virus | Vero | | 8.05 | >230 |
| La Crosse virus | Vero 76 | <0.23 | | >230 |
| La Crosse virus | Vero 76 | <0.23 | | 193 |
| La Crosse virus | Vero 76 | | 0.12 | >23 |
| La Crosse virus | Vero 76 | 0.69 | | >23 |
| Marpol virus | Vero | 25.3 | | >230 |
| Marpol virus | Vero | 19.1 | | >230 |
| Punta Toro virus | Vero 76 | 6.21 | | >230 |
| Punta Toro virus | Vero 76 | 5.75 | | >230 |
| Severe fever thrombocytopenia | Vero | | 7.82 | >230 |
| syndrome virus | | | | |

### Example 67. EIDD-2838 Arenaviridae Activity

| | Cell | EC₅₀ | EC₉₀ | CC₅₀ |
|---|---|---|---|---|
| Virus | Line | (μM) | (μM) | (μM) |
| Lassa fever virus | Vero | | 0.14 | >230 |
| Lassa fever virus | Vero | | 1.3 | 213 |
| Junin virus | Vero | 0.37 | | >230 |
| Junin virus | Vero | <0.23 | | >230 |
| Junin virus | Vero | | 0.023 | >23 |
| Junin virus | Vero | 0.023 | | >23 |
| Lymphocytic choriomeningitis virus | Vero | | 4.6 | >230 |
| Pichinde virus | Vero | 2.76 | | >230 |
| Pichinde virus | Vero | 1.45 | | >230 |
| Pichinde virus | Vero | | 0.55 | >23 |
| Pichinde virus | Vero | 0.35 | | >23 |
| Tacaribe virus | Vero | <0.23 | | >230 |
| Tacaribe virus | Vero | <0.23 | | >230 |
| Tacaribe virus | Vero | | 0.046 | >23 |
| Tacaribe virus | Vero | 0.046 | | >23 |

### Example 68. EIDD-2838 Influenza Activity

| | Cell | EC₅₀ | EC₉₀ | CC₅₀ |
|---|---|---|---|---|
| Virus | Line | (μM) | (μM) | (μM) |
| Influenza A H1N1 | MDCK | 1.2 | | >230 |
| Influenza A H1N1 | MDCK | 1.59 | | >230 |
| Flu A H7N9 (High Path) | MDCK | 5.29 | | >230 |
| Flu A H7N9 (High Path) | MDCK | 4.37 | | >230 |
| Flu A H5N1 (High Path) | MDCK | 23 | | >230 |
| Flu A H5N1 (High Path) | MDCK | 11.27 | | >230 |
| Flu A H1N1 | MDCK | 1.98 | | >230 |
| Flu A H1N1 | MDCK | 2.3 | | >230 |
| Flu A H3N2 | MDCK | 0.97 | | >230 |
| Flu A H3N2 | MDCK | 0.87 | | >230 |
| Flu A H5N1 (Low Path) | MDCK | 0.78 | | >230 |
| Flu A H5N1 (Low Path) | MDCK | 0.71 | | >230 |
| Flu B | MDCK | 0.78 | | >230 |
| Flu B | MDCK | 0.74 | | >230 |
| Flu B | MDCK | 0.74 | | >230 |
| Flu B | MDCK | 0.67 | | >230 |

### Example 69. EIDD-2838 Parainfluenza and RSV Activity

| | Cell | | | |
|---|---|---|---|---|
| Virus | Line | EC₅₀ (μM) | EC₉₀ (μM) | CC₅₀ (μM) |
| Parainfluenza virus 3 | MA-104 | 73.6 | | >230 |
| Parainfluenza virus 3 | MA-104 | 78.2 | | >230 |
| RSV | MA-104 | 3.45 | | >230 |
| RSV | MA-104 | 2.76 | | >230 |
| RSV | MA-104 | 7.82 | | >230 |
| RSV | MA-104 | 6.21 | | >230 |

### Example 70. EIDD-2838 Ebola Activity

| Virus | Cell Line | EC₅₀ (μM) | EC₉₀ (μM) | CC₅₀ (μM) |
|---|---|---|---|---|
| EBOV | Vero | 149.5 | | >230 |

### Example 71. EIDD-2838 Coronaviridae Activity

| Virus | Cell Line | EC₅₀ (μM) | EC₉₀ (μM) | CC₅₀ (μM) |
|---|---|---|---|---|
| MERS | Vero76 | 1.98 | | >230 |
| MERS | Vero E6 | 2.53 | | >230 |

### Example 72. EIDD-2838 Flaviviridae Activity

| Virus | Cell Line | EC₅₀ (μM) | EC₉₀ (μM) | CC₅₀ (μM) |
|---|---|---|---|---|
| POWV | BHK | 75.9 | | >230 |
| POWV | BHK | 98.9 | | >230 |
| ZIKV | Vero 76 | 7.82 | | >230 |
| ZIKV | Vero 76 | 9.2 | | >230 |
| DENV2 | Vero 76 | | 29.9 | >230 |
| DENV2 | Vero 76 | >230 | | >230 |
| JEV | Vero 76 | | 3.91 | >230 |
| JEV | Vero 76 | 78.2 | | >230 |
| Usutu virus | Vero 76 | | 25.76 | >230 |
| Usutu virus | Vero 76 | 6.9 | | >230 |
| ZIKV | Vero 76 | | 43.7 | 156.4 |
| ZIKV | Vero 76 | 8.28 | | 156.4 |

### Example 73. EIDD-2838 Picornaviridae Activity

| Virus | Cell Line | EC₅₀ (μM) | EC₉₀ (μM) | CC₅₀ (μM) |
|---|---|---|---|---|
| Poliovirus | Vero 76 | 7.36 | | >230 |
| Poliovirus | Vero 76 | 8.74 | | >230 |
| Coxsackie virus B3 | Vero 76 | 4.83 | | >230 |
| Coxsackie virus B3 | Vero 76 | 2.76 | | >230 |
| Enterovirus-68 | RD | 7.36 | | >230 |
| Enterovirus-68 | RD | 7.82 | | >230 |
| HRV-14 | HeLa-Ohio | >230 | | >230 |
| HRV-14 | HeLa-Ohio | >230 | | >230 |

### Example 74. EIDD-2749 Norovirus Activity

| Virus | Cell Line | EC₅₀ (μM) | EC₉₀ (μM) | CC₅₀ (μM) |
|---|---|---|---|---|
| Murine Norovirus | RAW264.7 | 2.2 | 7.2 | >100 |

### Example 75. Synthesis of ((2R,3S,4R,5R)-3,4-dihydroxy-5-(4-(hydroxyamino)-2-oxopyrimidin-1(2H)-yl)tetrahydrofuran-2-yl)methyl L-valinate hydrochloride

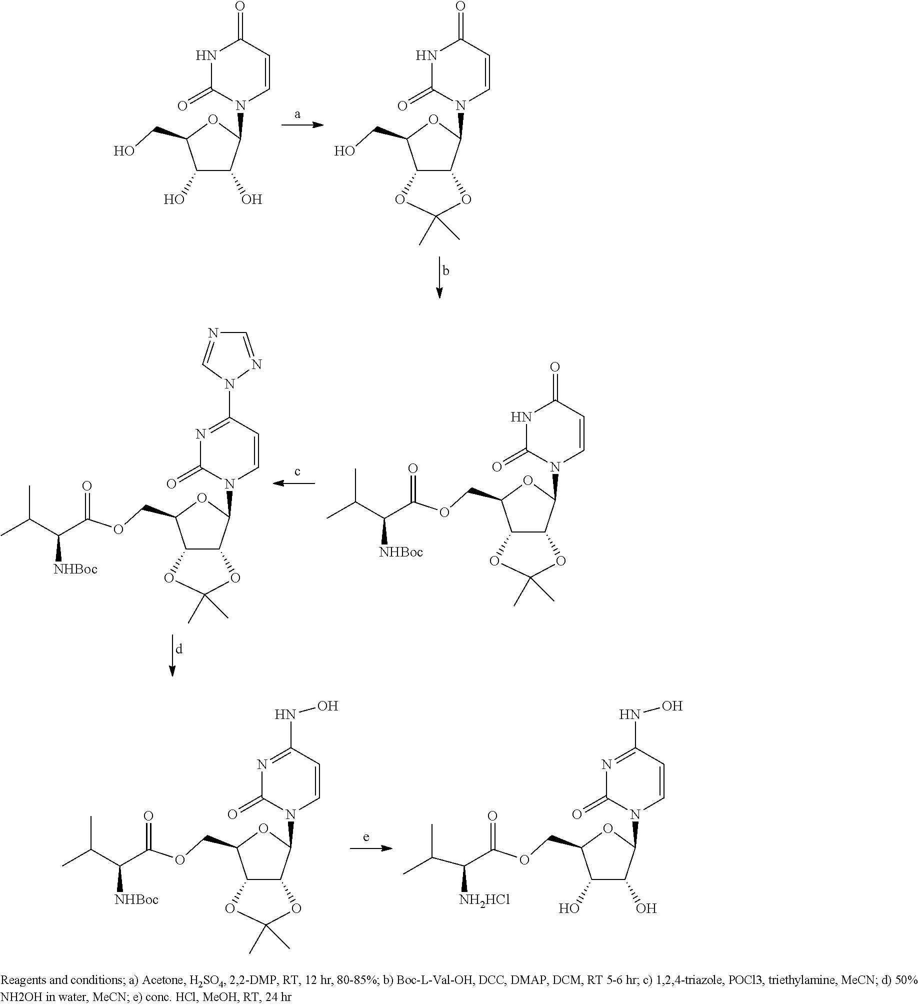

A 2 L 3-neck RBF was charged with 1-[(3R,4S,5R)-3,4-dihydroxy-5-(hydroxymethyl)tetrahydrofuran-2-yl]pyrimidine-2,4-dione (61.4 g, 251.43 mmol) and acetone (1400 mL). The resulting slurry was stirred at RT and sulfuric acid (2 mL was added. Stirring was continued overnite. The clear colorless solution was quenched/adjusted to basic pH with 100 mL of trimethylamine. The crude solution was concentrated under reduced pressure to yield a pale yellow oil. The residue was dissolved in 600 mL of EtOAc and washed with water×2, bicarb×2, water, brine×2 and dried over sodium sulfate. The colorless solution was concentrated under reduced pressure to yield 1-[(3aR,6R,6aR)-6-(hydroxymethyl)-2,2-dimethyl-3a,4,6,6a-tetrahydrofuro[3,4-d][1,3]dioxol-4-yl]pyrimidine-2,4-dione (45 g) as a white solid.

A 200 mL RBF was charged with 1-[(3aR,6R,6aR)-6-(hydroxymethyl)-2,2-dimethyl-3a,4,6,6a-tetrahydrofuro[3,4-d][1,3]dioxol-4-yl]pyrimidine-2,4-dione (2.36 g, 8.3 mmol) and DCM (50 mL). The reaction was stirred until a solution was formed. Next, (2S)-2-(tert-butoxycarbonylamino)-3-methyl-butanoic acid (2.16 g, 9.96 mmol) and N,N-dimethylpyridin-4-amine (0.1 g, 0.8300 mmol) were added. The reaction was cooled to 0° C. with an ice bath. A DCM solution of N,N′-dicyclohexylcarbodiimide (2.06 g, 9.96 mmol) was added slowly. The reaction mixture was allowed to warm to rt. Monitored by TLC (EtOAc).

A precipitate (DCU) formed after about 1 hr and no starting material was detected after 3 hrs. The solids were filtered off and rinsed with EtOAc. The filtrate was washed with water, brine, dried over sodium sulfate and concentrated under reduced pressure to yield white, gooey solid. The gummy solid was triturated with ether and filtered to remove the solid. The filtrate was concentrated under reduced pressure to yield about 8 g of thick viscous oil. The product was purified by SGC, pooled fractions 6-25 and concentrated under reduced pressure to yield [(3aR,6R,6aR)-4-(2,4-dioxopyrimidin-1-yl)-2,2-dimethyl-3a,4,6,6a-tetrahydrofuro[3,4-d][1,3]dioxol-6-yl]methyl (2S)-2-(tert-butoxycarbonylamino)-3-methyl-butanoate (3.8 g, 7.8592 mmol, 94.667% yield) as a foamy white solid after drying in vacuo.

1,2,4-triazole was taken in anhydrous acetonitrile and stirred at RT after 30 min, the reaction mixture was cooled to 0° C. and POCl₃ was added dropwise and continued stirring for 2 hr. After 2 hr triethylamine was added dropwise and continue stirring for 1 hr, the reaction mixture was slowly brought to RT, and the uridine derived substrate from the above reaction was added as solution in acetonitrile. The reaction mixture stirred at RT overnight. After completion of the reaction, the solvent was removed under reduced pressure and taken in DCM and extracted with water. The organic layer was dried over anhydrous sodium sulphate and concentrated under reduced pressure. The crude product was purified by flash column chromatography.

To a solution of the substrate in acetonitrile (10 mL/gm), 50% hydroxylamine in water was added dropwise and stirred at rt for 2-3 hrs. After completion of the reaction, solvent was removed under reduced pressure and the crude product was purified by flash column chromatography using hexane and EtOAc as eluent.

1 g of substrate was taken in 20 mL of methanol and treated with 2 mL of conc.HCl (36%) and after 3-4 hr 30% completion was observed. Another 5 mL of conc.HCl was added and stirred overnight. After completion of the reaction, solvent was removed and the crude product was taken in minimum methanol and added dropwise to excess diethylether with stirring, product was crashed out of solution and allowed to settle, ether was decanted and fresh ether was added, stirred, settled and decanted, the same process was repeated two times. After ether was decanted, solid was dried over a rotavap and high vacuum to get free flowing white solid. Ether was trapped in the solid and was difficult to remove. The solid was dissolved in methanol, evaporated and dried to get colorless foam, which still holds methanol. The foam was taken in water and a purple solution was observed. The purple solution was purified by reverse phase ISCO column chromatography using water and acetonitrile. The fractions containing product were evaporated under reduced pressure and lyophilized to get colorless solid.

### Example 76. Synthesis of EIDD-2800

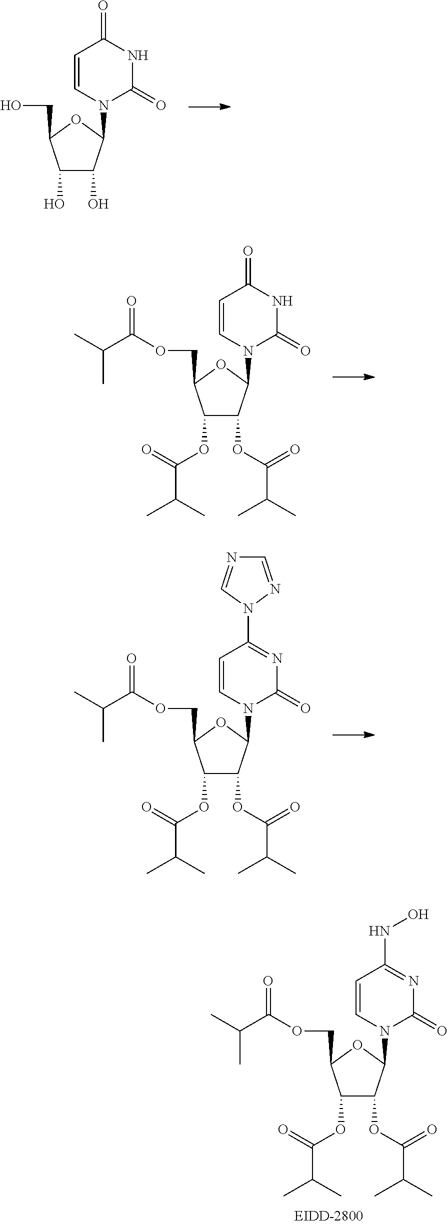

A 3-neck 1 L round bottom flask equipped with an overhead stirrer, temperature probe and addition funnel was charged with uridine (25 g, 102.38 mmol) and ethyl acetate (500 mL). The white slurry was stirred at ambient temperature while triethylamine (71.39 mL, 511.88 mmol) and DMAP (0.63 g, 5.12 mmol) were added to the mixture. The slurry was cooled in an ice bath and isobutyric anhydride (56.02 mL, 337.84 mmol) was slowly added to the reaction mixture over a 5 minute period. The temperature rose 25° C. during the addition. The resulting slurry was stirred at ambient temperature and monitored by TLC. After 1 hour, a clear colorless solution had formed and TLC showed no starting material. The reaction was quenched with 200 mL of water, stirred at rt for 20 minutes. The layers were separated, and the organics were washed with water (2×100 mL), saturated aqueous bicarbonate solution (100 mL×2), 100 mL of water, brine (100 mL×2), and then dried over sodium sulfate. The organics were filtered and the filtrate was concentrated under reduced pressure at 45° C. to yield a yellow oil. The oil was used in the next step without any further purification.

A 2 L 3-neck flask equipped with an argon inlet, overhead stirrer and temperature probe was charged with 1H-1,2,4-triazole (50.88 g, 736.68 mmol), triethylamine (114.17 mL, 818.54 mmol) and MeCN (350 mL). The reaction mixture was stirred at rt for 20 minutes. An ethyl acetate (350 mL) solution of [(2R,3R,4R)-5-(2,4-dioxopyrimidin-1-yl)-3,4-bis(2-methylpropanoyloxy)tetrahydrofuran-2-yl]methyl 2-methylpropanoate (46.5 g, 102.32 mmol) was added and the mixture was cooled to <5° C. using an ice bath. Stirring continued for 20 minutes. Next, phosphorous(V)oxychloride (14.35 mL, 153.48 mmol) was added slowly under argon at less than 20° C. over 15 minutes. The reaction was monitored by TLC (100% EtOAc), starting material (R_{f}=0.89) consumed in less than 2 hours and a new spot due to product (Rf=0.78) present. The reaction was quenched with 500 mL of water and 400 mL of EtOAc. The quenched reaction was allowed to stir at rt for 15 minutes. The layers were separated and the organic layer was washed with water (2×100 mL), 200 mL of 0.5N HCl, and brine (2×100 mL). The organics were dried over sodium sulfate, filtered and concentrated under reduced pressure to yield [(2R,3R,4R)-3,4-bis(2-methylpropanoyloxy)-5-[2-oxo-4-(1,2,4-triazol-1-yl)pyrimidin-1-yl]tetrahydrofuran-2-yl]methyl 2-methylpropanoate (49 g, 96.93 mmol, 94.735% yield) as a yellow oil. The crude material was used in the next step without further purification.

A 500 mL round bottom flask was charged with [(2R,3),4R)-3,4-bis(2-methylpropanoyloxy)-5-[2-oxo-4-(1,2,4-triazol-1-yl)pyrimidin-1-yl]tetrahydrofuran-2-yl]methyl 2-methylpropanoate (48.9 g, 96.73 mmol), ethyl acetate (400 mL), and isopropyl alcohol (100 mL). The reaction mixture was stirred at rt until all of the starting material was dissolved. The orange solution was treated with hydroxylamine (6.52 mL, 106.41 mmol), and the resulting pale yellow solution was stirred at rt and monitored by TLC (EtOAc). No starting material was observed after 1 hour. The reaction was quenched with 500 mL of water, and the layers were separated. The organics were washed with 100 mL of water, 100 mL×2 of brine, and then dried over sodium sulfate. The organics were filtered and concentrated under reduced pressure to yield the crude product. The crude product was dissolved in 180 mL of hot MTBE and allowed to cool to rt. Seed crystals were added, and the flask was placed in the freezer. The white solid that formed was collected by filtration, washed with a minimal amount of MTBE and dried in vacuo to yield the desired product.

### Example 77. Synthesis of EIDD-2801

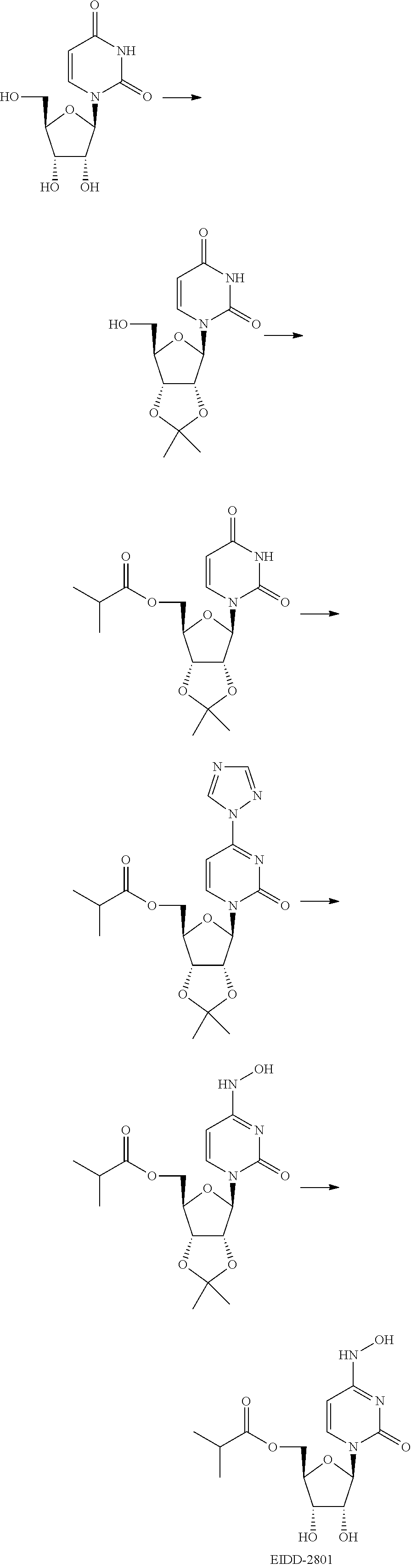

A 1 L round bottom flask was charged with uridine (25 g, 102.38 mmol) and acetone (700 mL). The reaction mixture was allowed to stir at rt. The slurry was then treated with sulfuric acid (0.27 mL, 5.12 mmol). Stirring was allowed to continue at rt for 18 hours. The reaction was quenched with 100 mL of trimethylamine and was used in the next step without further purification.

A 1 L round bottom flask was charged with the reaction mixture from the previous reaction. Triethylamine (71.09 mL, 510.08 mmol) and 4-dimethylaminopyridine (0.62 g, 5.1 mmol) were then added. The flask was cooled using an ice bath and then 2-methylpropanoyl 2-methylpropanoate (17.75 g, 112.22 mmol) was slowly added. The reaction mixture was allowed to stir at rt until the reaction was complete. The reaction mixture was concentrated under reduced pressure, and the residue was dissolved in 600 mL ethyl acetate and washed with saturated aqueous bicarbonate solution×2, water×2 and brine×2. The organics were dried over sodium sulfate and concentrated under reduced pressure to yield a clear colorless oil. The crude product was used in the next step without further purification.

A 1 L round bottom flask was charged with the crude product from above (36 g, 101.59 mmol) and MeCN (406.37 mL). The reaction mixture was allowed to stir until all the starting material was dissolved. Next, 1,2,4-triazole (50.52 g, 731.46 mmol) was added followed by the addition of N,N-diethylethanamine (113.28 mL, 812.73 mmol). The reaction mixture was allowed to stir at rt until all solids dissolved. The reaction was then cooled to 0° C. using an ice bath. Phosphorous oxychloride (24.44 mL, 152.39 mmol) was added slowly. The slurry that formed was allowed to stir under argon while slowly warming to rt. The reaction was then allowed to stir until complete by TLC (EtOAc). The reaction was then quenched by the addition of 100 mL of water. The slurry then became a dark colored solution, which was then concentrated under reduced pressure. The residue was dissolved in DCM and washed with water and brine. The organics were then dried over sodium sulfate, filtered, and concentrated under reduced pressure. The product was purified by silica gel chromatography (2×330 g columns). All fractions containing product were collected and concentrated under reduced pressure.

A 500 mL round bottom flask was charged with the product from the previous step (11.8 g, 29.11 mmol) and isopropyl alcohol (150 mL). The reaction mixture was allowed to stir at rt until all solids dissolved. Next, hydroxylamine (1.34 mL, 43.66 mmol) was added and stirring continued at ambient temperature. When the reaction was complete (HPLC) some solvent was removed under high vacuum at ambient temperature. The remaining solvent was removed under reduced pressure at 45° C. The resulting residue was dissolved in EtOAc and was washed with water and brine. The organics were dried over sodium sulfate, filtered, and concentrated under reduced pressure to yield oil. Crystals formed upon standing at rt. The crystals were collected by filtration, washed with ether×3, and dried in vacuo to provide the product as a white solid.

A 200 mL round bottom flask was charged with the product from the previous step (6.5 g, 17.6 mmol) and formic acid (100 mL, 2085.6 mmol). The reaction mixture was allowed to stir at rt overnight. The progress of the reaction was monitored by HPLC. The reaction mixture was concentrated under reduced pressure at 42° C. to yield a clear, pale pink oil. Next, 30 mL of ethanol was added. Solvent was then removed under reduced pressure. MTBE (50 mL) was added to the solid and heated. Next, isopropyl alcohol was added and heating was continued until all solid material dissolved (5 mL). The solution was then allowed to cool and stand at rt. A solid started to form after about hr. The solids were collected by filtration, washed with MTBE, and dried in vacuo to yield the EIDD-2801 as a white solid. The filtrate was concentrated under reduced pressure to yield a sticky solid, which was dissolved in a small amount of isopropyl alcohol with heating. The solution was allowed to stand at rt overnight. A solid formed in the flask, which was collected by filtration, rinsed with isopropyl alcohol and MTBE, and dried in vacuo to an additional crop of desired product.

EIDD-2801 (25 g) was dissolved in 250 mL of isopropyl alcohol by heating to 70° C. to give a clear solution. The warm solution was polish filtered and filtrate transferred to 2 L three neck flask with overhead stirrer. It was warmed back to 70° C. and MTBE (250 mL) was slowly added into the flask. The clear solution was seeded and allowed to cool slowly to rt with stirring for 18 hrs. The EIDD-2801 solid that formed was filtered and washed with MTBE and dried at 50° C. under vacuum for 18 hours. The filtrate was concentrated, redissolved in 50 mL isopropyl alcohol and 40 mL MTBE by warming to give clear solution and allowed to stand at rt to give a second crop of EIDD-2801.

### Example 78. Norovirus Activity for EIDD-2749

| | | | EC₅₀ | EC₉₀ | CC₅₀ |
|---|---|---|---|---|---|
| Virus | Strain | Cell Line | (μM) | (μM) | (μM) |
| Murine Norovirus | MNV-1 | RAW264.7 | 2.2 | 7.2 | >100 |
| Human Norovirus | G1 | HG23 | 2.7 | 9.3 | 100 |

### Example 78. Togaviridae Activity for EIDD-2749

| | | | EC₅₀ | EC₉₀ | CC₅₀ |
|---|---|---|---|---|---|
| Virus | Strain | Cell Line | (μM) | (μM) | (μM) |
| CHIKV | S27 (VR-64) | Vero76 | 1.98 | | >380 |
| CHIKV | S27 (VR-64) | Vero76 | 2.05 | | >380 |
| CHIKV | S27 (VR-64) | Vero 76 | | 9.12 | >380 |
| CHIKV | S27 (VR-64) | Vero 76 | 3.42 | | >380 |
| VEEV | TC-83 | Huh-7 | 1.6 | | >380 |
| VEEV | TC-83 | Huh-7 | 19 | | >380 |
| VEEV | TC-83 | Vero 76 | | 213 | >380 |
| VEEV | TC-83 | Vero 76 | 7.6 | | >380 |
| EEEV | FL93-939 | Vero76 | 380 | | >380 |
| EEEV | FL93-939 | Vero76 | >380 | | >380 |
| WEEV | California | Vero76 | 9.88 | | >380 |
| WEEV | California | Vero76 | 11.8 | | >380 |

### Example 80. Flaviviridae Activity for EIDD-2749

| | | | EC₅₀ | EC₉₀ | CC₅₀ |
|---|---|---|---|---|---|
| Virus | Strain | Cell Line | (μM) | (μM) | (μM) |
| DENV2 | New Guinea C | Huh-7 | >380 | | >380 |
| DENV2 | New Guinea C | Huh-7 | >380 | | >380 |
| ZIKV | MR766 | Vero 76 | 16.3 | | >380 |
| ZIKV | MR766 | Vero 76 | 3.8 | | >380 |
| ZIKV | MR766 | Vero 76 | | >380 | >380 |
| ZIKV | MR766 | Vero 76 | 28.1 | | >380 |

### Example 81. Picornaviridae Activity for EIDD-2749

| Virus | Strain | Cell Line | EC₅₀ (μM) | EC₉₀ (μM) | CC₅₀ (μM) |
|---|---|---|---|---|---|
| ENTV71 | Tainan/4643/98 | Vero 76 | 3.27 | | >380 |
| ENTV71 | Tainan/4643/98 | Vero 76 | 1.33 | | >380 |
| Coxsackie virus B3 | HA201933 | Vero 76 | 3.12 | | >380 |
| Coxsackie virus B3 | HA201933 | Vero 76 | 3.15 | | >380 |
| Coxsackie virus B3 | HA201933 | Vero 76 | | >380 | >380 |
| Coxsackie virus B3 | HA201933 | Vero 76 | 12 | | >380 |
| Enterovirus-68 | US/KY/14-18953 | RD | 13.7 | | >380 |
| Enterovirus-68 | US/KY/14-18953 | RD | 11.8 | | >380 |
| Poliovirus | Mahoney | Vero 76 | 12.2 | | >380 |
| Poliovirus | Mahoney | Vero 76 | 11.8 | | >380 |

### Example 82. Respiratory Virus Activity for EIDD-2749

| Virus | Strain | Cell Line | EC₅₀ (μM) | CC₅₀ (μM) |
|---|---|---|---|---|
| Influenza A H1N1 | CA/07/20/09 | MDCK | <0.38 | >380 |
| Influenza A H1N1 | CA/07/20/09 | MDCK | <0.38 | >380 |
| Flu A H3N2 | Perth/16/2009 | MDCK | <0.38 | >380 |
| Flu A H3N2 | Perth/16/2009 | MDCK | <0.38 | >380 |
| Flu A H5N1 (Low Path) | Duck/MN/1525/81 | MDCK | <0.38 | >380 |
| Flu A H5N1 (Low Path) | Duck/MN/1525/81 | MDCK | <0.38 | >380 |
| Flu B | Brisbane/60/2008 | MDCK | <0.38 | >380 |
| Flu B | Brisbane/60/2008 | MDCK | <0.38 | >380 |
| Flu B | Florida/4/2006 | MDCK | <0.38 | >380 |
| Flu B | Florida/4/2006 | MDCK | <0.38 | >380 |
| Parainfluenza virus 3 | 14702 | MA-104 | 98.8 | >380 |
| Parainfluenza virus 3 | 14702 | MA-104 | 60.8 | >380 |
| HRV-14 | 1059 | HeLa-Ohio | 1.22 | 32.7 |
| HRV-14 | 1059 | HeLa-Ohio | 1.14 | 31.9 |
| RSV | A2 | MA-104 | 1.22 | >380 |
| RSV | A2 | MA-104 | <0.38 | >380 |

### Example 83. Coronavirus Activity for EIDD-2749

| Virus | Strain | Cell Line | EC₅₀ (μM) | CC₅₀ (μM) |
|---|---|---|---|---|
| MERS | EMC | Vero76 | >380 | >380 |
| MERS | EMC | Vero76 | >380 | >380 |

### Example 84. Bunyaviridae Activity for EIDD-2749

| Virus | Strain | Cell Line | EC₅₀ (μM) | EC₉₀ (μM) | CC₅₀ (μM) |
|---|---|---|---|---|---|
| RVFV | MP-12 | Vero76 | 3.46 | | >380 |
| RVFV | MP-12 | Vero76 | 2.96 | | >380 |
| RVFV | MP-12 | Vero 76 | | 23.9 | >380 |
| RVFV | MP-12 | Vero 76 | 16.3 | | >380 |
| RVFV | MP-12 | Vero 76 | | 4.3 | >380 |
| Heartland virus | MO-4 | Vero | | 121.6 | >380 |
| La Crosse virus | Wisconsin 1960 (VR-744) | Vero 76 | <0.38 | | >380 |
| La Crosse virus | Wisconsin 1960 (VR-744) | Vero 76 | <0.38 | | >380 |
| La Crosse virus | Wisconsin 1960 (VR-744) | Vero 76 | | 0.27 | >380 |
| La Crosse virus | Wisconsin 1960 (VR-744) | Vero 76 | 0.38 | | >380 |
| Marpol virus | HV97021050 | Vero E6 | 95 | | >380 |
| Marpol virus | HV97021050 | Vero E6 | 57 | | >380 |
| Punta Toro virus | Adames | Vero 76 | 72.2 | | >380 |
| Punta Toro virus | Adames | Vero 76 | 91.2 | | >380 |
| Severe fever | HB-29 | Vero | | >380 | >380 |
| thrombocytopenia | | | | | |
| syndrome virus | | | | | |

### Example 85. Arenaviridae Activity for EIDD-2749

| Virus | Strain | Cell Line | EC₅₀ (μM) | EC₉₀ (μM) | CC₅₀ (μM) |
|---|---|---|---|---|---|
| Tacaribe virus | TRVL 11573 | Vero | <0.38 | | >380 |
| Tacaribe virus | TRVL 11573 | Vero | <0.38 | | >380 |
| Tacaribe virus | TRVL 11573 | Vero | | 0.87 | >380 |
| Tacaribe virus | TRVL 11573 | Vero | 0.34 | | >380 |
| Lassa fever virus | Josiah | Vero | 0.38 | | >380 |
| Lassa fever virus | Josiah | Vero | | 9 | >380 |
| Lymphocytic | Armstrong | Vero | | 7.22 | >380 |
| choriomeningitis virus | | | | | |
| Pichinde virus | An 4763 | Vero | 2.81 | | >380 |
| Pichinde virus | An 4763 | Vero | 1.03 | | >380 |
| Pichinde virus | An 4763 | Vero | | 0.19 | >380 |
| Junin virus | Candid #1 | Vero | 1.86 | | >380 |
| Junin virus | Candid #1 | Vero | 1.29 | | >380 |
| Junin virus | Candid #1 | Vero | | 0.38 | >380 |

### Example 86. Filovirus Activity for EIDD-2749

| | | Cell | EC₅₀ | EC₉₀ | CC₅₀ |
|---|---|---|---|---|---|
| Virus | Strain | Line | (μM) | (μM) | (μM) |
| EBOV | Zaire | Vero | 1.52 | | >380 |
| EBOV | Zaire | Vero | | 22 | >380 |
| Marburg virus | Angola | Vero | >380 | | >380 |

### Example 87. EIDD-2749 Cytotoxicity

| ID | Cell Line | CC50 |
|---|---|---|
| EIDD-02749 | CEM | 107.3 |
| EIDD-02749 | HepG2 | >400 |
| EIDD-02749 | Huh-7 | >400 |
| EIDD-02749 | A-204 | >400 |
| EIDD-02749 | A549 | >400 |
| EIDD-02749 | BxPc3 | >400 |
| EIDD-02749 | H9c-2 | >400 |
| EIDD-02749 | IEC-6 | 371.7 |
| EIDD-02749 | Vero | >400 |

### Example 88. Synthesis of EIDD-2749-5′-Monophosphate (EIDD-02986)

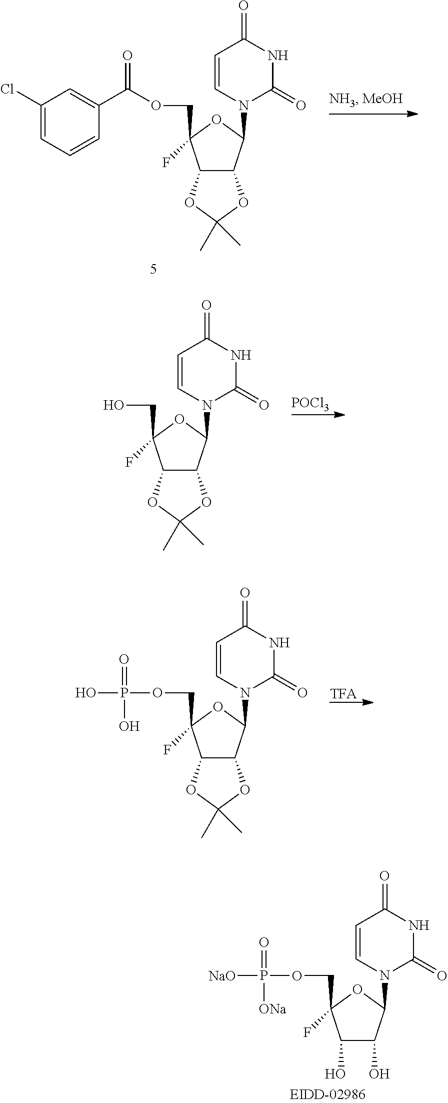

A heavy wall 350 mL round-bottomed pressure vessel was charged with 5′-(3-chlorobenzoyloxy)-4′-fluoro-2′,3′-O-isopropylideneuridine (4.1 g, 9.3 mmol) and 7N ammonia in methanol (66 mL, 462 mmol). The mixture was stirred for 6 h at room temperature after which time tlc indicated complete consumption of starting material. The mixture was concentrated in vacuo, and the resulting residue purified by column chromatography over silica gel (40 g) eluting with a methylene chloride/methanol gradient to give 4′-fluoro-2′,3′-O-isopropylideneuridine (2.5 g, 89%) as a white solid.

¹H NMR (400 MHz, Chloroform-d) δ 9.24 (s, 1H), 7.23 (d, J=8.0 Hz, 1H), 5.77 (d, J=8.0 Hz, 1H), 5.72 (s, 1H), 5.24 (dd, J=12.6, 6.5 Hz, 1H), 5.07 (dd, J=6.4, 1.3 Hz, 1H), 2.50 (s, 1H), 1.59 (s, 3H), 1.38 (s, 3H).

¹⁹F NMR (376 MHz, Chloroform-d) δ −115.53 (dd, J=12.4, 8.8 Hz).

A solution of tristriazolide in acetonitrile was freshly prepared by treating a mixture of 1,2,4-triazole (468.91 mg, 6.79 mmol) and triethylamine (0.95 mL, 6.79 mmol) in acetonitrile (7.5 mL) dropwise with phosphorus oxychloride (0.21 mL, 2.27 mmol) over a 5 min period at−15° C. After stirring for an additional 20 min at −15° C., the triethylammonium precipitate was removed by centrifuge, and the solution of tristriazolide was added to an acetonitrile solution (7.5 mL) of 4′-fluoro-2′,3′-O-isopropylideneuridine (225 mg, 0.74 mmol) at −15° C. After stirring for 15 min at −15° C., the mixture was allowed to warm to rt, and continued for another 1.5 hr. The mixture was quenched with 50 mM TEAB (30 mL), stirred for 1 h at rt, and concentrated to dryness in vacuo. The resulting residue was co-evaporated with water (2×20 mL) and purified by ion-exchange chromatography over DEAE-Sephadex A-25 (HCO₃⁻ form) eluting with a gradient from 0 to 0.2 M (700 mL) aqueous ammonium bicarbonate in 10% ethanol. Fractions were analyzed by tlc (7:2:1 iPa:NH₄OH:water), and target fractions combined and concentrated. The product was further purified by reversed-phase chromatography with a CombiFlash equipped with a C-18 column (50 g) eluting with 0.01M aqueous ammonium bicarbonate. Product containing fractions were pooled, frozen, and concentrated by lyophilization to give 4′-fluoro-2′,3′-O-isopropylideneuridine 5′-O-phosphate (131 mg, 46%) as a white solid.

¹H NMR (400 MHz, D₂O) δ 7.64 (d, J=8.0 Hz, 1H), 6.08 (s, 1H), 5.81 (d, J=7.8 Hz, 1H), 5.21 (dd, J=12.4, 6.6 Hz, 1H), 5.14 (d, J=6.5 Hz, 1H), 4.02-3.73 (m, 2H), 1.54 (s, 3H), 1.36 (s, 3H).

³¹P NMR (162 MHz, D₂O) δ 3.46.

¹⁹F NMR (376 MHz, D₂O) δ −113.90 (q, J=12.4, 11.9 Hz).

A 50 mL round-bottomed flask was charge with 4′-fluoro-2′,3′-O-isopropylideneuridine-5′-O-phosphate (171 mg, 0.43 mmol), water (0.5 mL) and acetic acid (1.5 mL). The solution was cooled to 10° C. and treated with cold aqueous 90% trifluoroacetic acid (3.3 mL, 43.15 mmol). After 5 min, the mixture was allowed to warm to room temperature and stirred an additional 2 h. The mixture was concentrated in vacuo and the resulting gum co-evaporated with water (5×10 mL) followed by methanol (3×10 ml). The crude product as a solution in methanol (10 mL) was filtered, concentrated to approximately 4 mL in volume and treated with a cold solution of 1M sodium perchlorate in acetone (20 mL). After 20 min at 0° C., the white precipitate was collected by centrifuge. The white solid was washed with acetone (5×14 mL), dissolved in water (4 mL) and concentrated by lyophilization to give 4′-fluorouridine-5′-monophosphate (EIDD-02986) (78 mg, 45%) as the disodium form.

¹H NMR (400 MHz, D₂O) δ 7.75 (d, J=8.1 Hz, 1H), 6.09 (s, 1H), 5.92-5.82 (m, 1H), 4.58-4.49 (m, 1H), 4.42 (dd, J=6.4, 1.9 Hz, 1H), 4.11 (t, J=5.2 Hz, 3H).

³¹P NMR (162 MHz, D₂O) δ −0.27.

¹⁹F NMR (376 MHz, D₂O) δ −121.26 (dt, J=19.1, 5.1 Hz).

LCMS Calculated for C₉H₁₁FN₂O₉P [M−H⁺]: 341.0; found: 340.9.

### Example 89. Synthesis of EIDD-2749-5′-triphosphate (EIDD-02991)

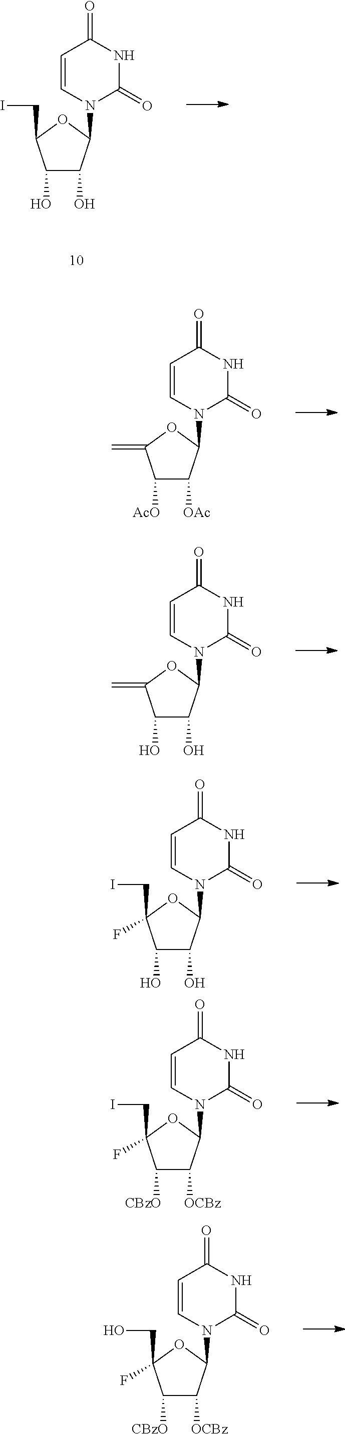

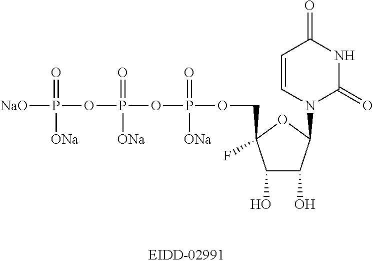

A 2 L three-necked round-bottomed flask flushed with argon and fitted with a mechanical stirrer, thermometer was charged with 5′-deoxy-5′-iodouridine (80 g, 225.92 mmol) and dry methanol (500 mL). Under argon atm, the white suspension was treated with a solution of 25% (4.37 M) sodium methoxide in methanol (103.4 mL, 451.85 mmol). The resulting homogeneous solution was stirred at 60° C. for 3 h. Methanol was removed in vacuo, and the resulting residue dissolved in anhydrous acetonitrile (300 mL). After addition of acetic anhydride (70.2 mL, 743 mmol), the mixture was heated to 60° C. for 5 h. Once cooled to room temperature, the mixture was concentrated in vacuo, and the resulting residue dissolved in ethyl acetate (500 mL) and treated with saturated sodium bicarbonate (100 mL). The organic layer was separated, washed with brine (100 mL), dried and concentrated to dryness to give 2′,3′-di-O-acetyl-4′,5′-didehydro-5′-deoxyuridine (70 g, 99% yield).

¹H NMR (400 MHz, DMSO-d₆) δ 11.53 (d, J=1.9 Hz, 1H), 7.75 (d, J=8.1 Hz, 1H), 6.07 (d, J=4.3 Hz, 1H), 5.92 (d, J=6.5 Hz, 1H), 5.69 (dd, J=8.0, 1.8 Hz, 1H), 5.63 (dd, J=6.4, 4.3 Hz, 1H), 4.52 (t, J=1.9 Hz, 1H), 4.28 (d, J=2.4 Hz, 1H), 2.08 (s, 3H), 2.04 (s, 3H).

In a 1 L round-bottomed flask, a solution of 2′,3′-di-O-acetyl-4′,5′-didehydro-5′-deoxyuridine (70 g, 225.6 mmol) in methanol (350 mL) was treated with 30% ammonium hydroxide (85.3 mL, 2190.7 mmol). After 18 h at room temperature, the mixture was concentrated in vacuo and the resulting residue dissolved in a 65:35:5 mixture of acetonitrile:isopropanol:methanol. After 30 min, the white precipitate was collected by vacuum filtration and washed with acetonitrile and hexanes. A second crop was isolated by concentrating the filtrate and stirring the resulting solid with acetonitrile. Combined crops were dried under high vacuum for 18 h to give 4′,5′-didehydro-5′-deoxyuridine (35 g. 68% yield) as a white solid.

¹H NMR (400 MHz, DMSO-d₆) δ 11.44 (s, 1H), 7.59 (d, J=8.1 Hz, 1H), 5.96 (d, J=5.4 Hz, 1H), 5.64 (d, J=8.1 Hz, 1H), 5.60 (d, J=5.8 Hz, 1H), 5.46 (d, J=5.7 Hz, 1H), 4.38 (t, J=5.5 Hz, 1H), 4.33 (s, 1H), 4.24 (q, J=5.5 Hz, 1H), 4.17 (d, J=1.8 Hz, 1H).

A 2 L three-necked round-bottomed flask was charged with 4′,5′-didehydro-5′-deoxyuridine (35 g, 154.7 mmol) and anhydrous acetonitrile (400 mL). The suspension was cooled to 0° C. under argon atm and treated with triethylamine trihydrofluoride (12.6 mL, 77.4 mmol) followed by the addition of N-iodosuccinimide (45.3 g, 201.2 mmol). After 1 h at 0° C., tlc (10% methanol in methylene chloride) indicated complete conversion. While still cold, the mixture was vacuum filtered. The isolated solid was washed sequentially with acetonitrile, dichloromethane, hexanes, and then dried under high vacuum for 18 h to give 5′-deoxy-4′-fluoro-5′-iodouridine (35 g, 61%).

¹H NMR (400 MHz, Methanol-d₄) δ 7.77 (d, J=8.1 Hz, 1H), 6.05 (s, 1H), 5.69 (d, J=8.1 Hz, 1H), 4.43 (dd, J=18.2, 6.5 Hz, 1H), 4.25 (d, J=6.6 Hz, 1H), 3.85-3.63 (m, 2H).

¹⁹F NMR (376 MHz, Methanol-d₄) δ −112.49 (ddd, J=20.9, 18.1, 6.1 Hz).

A 150 mL round-bottomed flask was charged with 5′-deoxy-5′-iodo-4′-fluorouridine (2.6 g, 6.99 mmol) and methylene chloride (35 mL). After stirring for 20 min at room temperature, the suspension was cooled to 0° C. and treated with benzyl chloroformate (4.49 mL, 31.44 mmol) followed by dropwise addition of 1-methylimidazole (3.34 mL, 41.93 mmol) over a 10 min period. The mixture was stirred an additional 10 min at 0° C. and then allowed to slowly warm to room temperature. After 18 h, the turbid mixture was diluted with methylene chloride (120 mL) and washed with 0.5M HCl solution (75 mL), water (50 mL), and brine (50 mL). The organic layer was separated, dried and concentrated in vacuo. The resulting residue was purified by column chromatography over silica gel (80 g) eluting with a methylene chloride/methanol gradient. Pure fractions were combined and concentrated in vacuo to give 2′,3′-di-O-benzyloxycarbonyl-5′-deoxy-4′-fluoro-5′-iodouridine (4.2 g, 94% yield) as a white solid.

¹H NMR (400 MHz, CDCl₃) δ 9.02 (s, 1H), 7.44-7.28 (m, 10H), 7.14 (d, J=8.0 Hz, 1H), 5.86-5.72 (m, 2H), 5.69-5.57 (m, 2H), 5.19 (d, J=4.3 Hz, 2H), 5.09 (d, J=3.1 Hz, 2H), 3.71-3.35 (m, 2H).

¹⁹F NMR (376 MHz, CDCl₃) δ −107.06 (td, J=18.6, 7.3 Hz).

In a 100 mL round-bottomed flask a 55% tetrabutylammonium hydroxide solution in water (8.04 mL, 9.37 mmol) was adjusted to pH 3.5 by dropwise addition of trifluoroacetic acid (0.72 mL, 9.37 mmol) while maintaining a temperature below 25° C. The mixture was then treated with a methylene chloride (15 mL) solution of 2′,3′-di-O-benzyloxycarbonyl-5′-deoxy-4′-fluoro-5′-iodouridine (2 g, 3.12 mmol) followed by addition of 3-chloroperbenzoic acid (3.6 g, 15.62 mmol) in portions over a 30 min period. After one hour the pH drifted to pH 1.4. The mixture was adjusted back to pH 3.5 with 1N sodium hydroxide and allowed to stir for 16 h after which time tlc (10% methanol in methylene chloride) and LCMS indicated complete conversion. The reaction mixture was quenched by addition of sodium thiosulfate (3.21 g, 20.31 mmol) slowly in portions while maintaining a temperature below 25° C. After stirring for 30 min, the methylene chloride layer was separated, and the aqueous layer extracted with additional methylene chloride (2×30 mL). Combined organic layers were dried over sodium sulfate, concentrated, and purified by column chromatography over silica gel (80 g) eluting with 60% ethyl acetate in hexanes followed by a second column of silica gel (80 g) eluting with a methylene chloride/methanol gradient to give 2′,3′-di-O-benzyloxycarbonyl-4′-fluorouridine (1.05 g, 63% yield) as a white solid.

¹H NMR (400 MHz, CDCl₃) δ 9.30 (s, 1H), 7.39-7.29 (m, 10H), 7.21 (d, J=8.1 Hz, 1H), 5.83 (dd, J=17.8, 7.0 Hz, 1H), 5.77-5.71 (m, 2H), 5.61 (dd, J=7.0, 2.4 Hz, 1H), 5.17 (d, J=4.8 Hz, 2H), 5.09 (s, 2H), 3.86 (q, J=5.8, 4.9 Hz, 2H), 3.06 (s, 1H).

¹⁹F NMR (376 MHz, CDCl₃) δ −121.03 (dt, J=17.7, 4.6 Hz).

A 10 mL round-bottomed flask charged with 2′,3′-di-O-benzyloxycarbonyl-4′-fluorouridine (348 mg, 0.66 mmol) and anhydrous trimethyl phosphate (3.5 mL). After stirring for 20 min at room temperature, the solution was cooled to 0° C. and treated with 1-methylimidazole (115 μL, 1.44 mmol) followed by dropwise addition of phosphorus oxychloride (122 μL, 1.31 mmol) over a 40 min period. The mixture continued to stir at 0° C. for 3.5 h after which time tlc (10% methanol in DCM and then 7:2:1 iPa:NH₄OH:water) indicated complete phosphorylation. The mixture was treated with tributylamine (0.94 mL, 3.94 mmol), tris(tetrabutylammonium)pyrophosphate (887 mg, 0.98 mmol), and anhydrous DMF (1.5 mL). After 1 h at room temperature, the reaction mixture was quenched with 100 mM TEAB (20 mL), stirred for 1 h, degassed by pump-fill with argon (3×) and treated with 10% palladium on carbon (100 mg). After cooling with an ice-bath, the mixture was pump-filled with hydrogen (2×) followed by vigorous stirring under atm pressure of hydrogen for 30 min. The mixture was pump-filled with argon and then vacuum filtered through a pad of Celite. The palladium was washed with water (2×20 mL). Combined filtrates were washed with ether (4×60 mL) and then concentrated in vacuo at 25° C. The residue was co-evaporated with water (2×25 mL) and purified by column chromatography over DEAE-Sephadex GE A-25 (10 mm×130 mm) eluting with a gradient from 100 mM to 500 mM TEAB (900 mL). Pure fractions as determined by tlc (8:1:1 NH₄OH:iPrOH:water) were combined and concentrated in vacuo with the bath temperature set at 25° C. The resulting solid was dissolved in methanol (1 mL) and treated with saturated solution of sodium perchlorate in acetone (10 mL). The resulting white precipitate was collected by centrifuge and washed with acetone (5×5 mL). The solid was dissolved in water (1 mL), frozen and lyophilized to yield 4′-fluorouridine 5′-O-triphosphate (3.14 mg, 0.81% yield) as the tetrasodium form.

¹H NMR (400 MHz, D₂O) δ 7.77 (d, J=8.0 Hz, 1H), 6.15 (d, J=1.9 Hz, 1H), 5.91 (d, J=8.1 Hz, 1H), 4.72-4.57 (m, 1H), 4.41 (d, J=6.3 Hz, 1H), 4.30 (ddd, J=10.2, 6.3, 3.0 Hz, 1H), 4.17 (dt, J=10.8, 5.0 Hz, 1H).

³¹P NMR (162 MHz, D₂O) δ −7.81(d), −11.84 (d, J=19.2 Hz), −22.23 (t).

¹⁹F NMR (376 MHz, D₂O) δ −121.09 (unresolved dt, J=19.2 Hz).

LCMS Calculated for C₉H₁₃FN₂O₁₅P₃[M−H⁺]: 500.9; found: 500.8.

### Example 90. Synthesis of EIDD-2749-5′-Isobutyl ester (EIDD-02947)

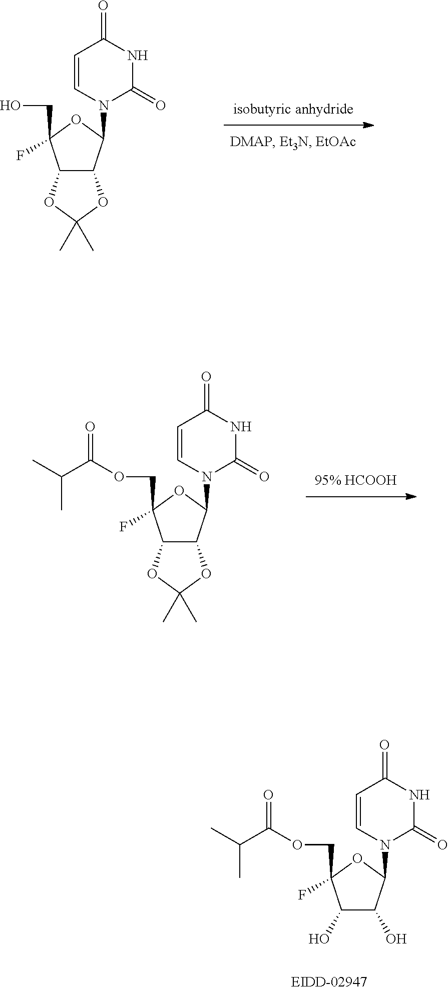 [SMILES: CC1(C)O[C@H]2[C@H](n3ccc(=O)[nH]c3=O)O[C@](F)(CO)[C@H]2O1 ; CC(C)C(=O)OC[C@@]1(F)O[C@@H](n2ccc(=O)[nH]c2=O)[C@@H]2OC(C)(C)O[C@@H]21 ; CC(C)C(=O)OC[C@@]1(F)O[C@@H](n2ccc(=O)[nH]c2=O)[C@H](O)[C@@H]1O]

To a 25 mL pear-shaped flask charged with 1-[(3aS,4S)-4-fluoro-4-(hydroxymethyl)-2,2-dimethyl-6,6a-dihydro-3aH-furo[3,4-d][1,3]dioxol-6-yl]pyrimidine-2,4-dione (0.1 g, 0.33 mmol) and DMAP (2.0 mg, 0.02 mmol) was added EtOAc (1.1 mL) to give a colorless solution. The vessel was vacuumed and charged with argon. Then Et₃N (083.12 mL, 0.83 mmol) was added, followed by isobutyric anhydride (0.07 mL, 0.4 mmol). This reaction solution was allowed to stir at room temperature overnight. After overnight stirring, TLC showed no SM. The reaction solution was transferred into a separation funnel and water was added. The aqueous layer was separated and re-extracted with DCM once. The combined organic layers was dried (Na₂SO₄), filtered and concentrated in vacuo. The crude material was purified by ISCO column chromatography (12 g) eluting from 100% hexanes to 80% EtOAc in hexanes to afford [(3aS,4S)-6-(2,4-dioxopyrimidin-1-yl)-4-fluoro-2,2-dimethyl-6,6a-dihydro-3aH-furo[3,4-d][1,3]dioxol-4-yl]methyl 2-methylpropanoate (0.11 g, 89%) as a white glassy solid. To a 25 mL pear-shaped flask charged with [(3aS,4S)-6-(2,4-dioxopyrimidin-1-yl)-4-fluoro-2,2-dimethyl-6,6a-dihydro-3aH-furo[3,4-d][1,3]dioxol-4-yl]methyl 2-methylpropanoate (0.11 g, 0.3000 mmol) was added 95% formic acid (12 mL, 0.3 mmol) to give a colorless solution. After stirring at room temperature for 3.5 h, solvent was removed in vacuo. Then water and Celite were added, concentrated in vacuo. The crude material was purified by ISCO column chromatography (12 g) eluting from 100% DCM to 15% MeOH in DCM to afford the product with some impurity. This material was re-purified by ISCO column chromatography (12 g) eluting from 100% hexanes to 100% EtOAc to afford [(2S,3S)-5-(2,4-dioxopyrimidin-1-yl)-2-fluoro-3,4-dihydroxy-tetrahydrofuran-2-yl]methyl 2-methylpropanoate (EIDD-02947) (7.8 mg, 8% yield) after lyophilizing overnight as a white fluffy solid.

¹H NMR (400 MHz, Methanol-d₄) δ 7.59 (d, J=8.1 Hz, 1H), 5.87 (d, J=2.1 Hz, 1H), 5.69 (d, J=8.1 Hz, 1H), 4.51 (dd, J=19.2, 6.9 Hz, 1H), 4.44-4.34 (m, 2H), 4.28 (dd, J=11.9, 8.1 Hz, 1H), 2.72-2.53 (m, 1H), 1.17 (dd, J=7.0, 4.8 Hz, 6H).

¹⁹F NMR (376 MHz, Methanol-d₄) δ −123.39 (dt, J=19.1, 8.0 Hz).

¹³C NMR (101 MHz, CD₃OD) δ 176.24, 164.59, 150.18, 142.88, 142.80, 116.65, 114.36, 101.82, 101.62, 95.71, 95.46, 70.98, 70.88, 70.27, 70.07, 61.44, 61.02, 33.66, 33.51, 17.90, 17.84, 17.80.

### Example 92. Synthesis of EIDD-2749-5′-L-Valine ester (EIDD-02971)

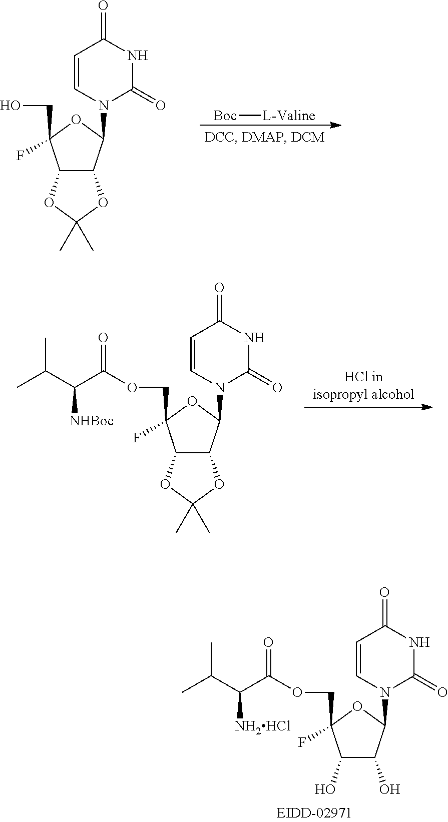

To a 25 mL pear-shaped flask charged with 1-[(3aS,4S,6R,6aR)-4-fluoro-4-(hydroxymethyl)-2,2-dimethyl-6,6a-dihydro-3aH-furo[3,4-d][1,3]dioxol-6-yl]pyrimidine-2,4-dione (0.15 g, 0.5 mmol), Boc-L-Valine (0.13 g, 0.6 mmol) and DMAP (0.01 g, 0.05 mmol) was added dry DCM (2 mL) to give a colorless solution. The reaction vessel was vacuumed and charged with argon. Then DCC (0.12 g, 0.6 mmol) was added all at once to give a white suspension. After overnight stirring, the white suspension was filtered through Celite and the solids were washed with DCM. Celite was added to the filtrate, and the filtrate was then concentrated in vacuo. The crude material was purified by ISCO column chromatography (24 g) eluting from 100% hexanes to 100% EtOAc to afford [(3aS,4S,6R,6aR)-6-(2,4-dioxopyrimidin-1-yl)-4-fluoro-2,2-dimethyl-6,6a-dihydro-3aH-furo[3,4-d][1,3]dioxol-4-yl]methyl (2S)-2-(tert-butoxycarbonylamino)-3-methyl-butanoate (0.158 g, 63%).

To a 10 mL pear-shaped vial charged with [(3aS,4S,6R,6aR)-6-(2,4-dioxopyrimidin-1-yl)-4-fluoro-2,2-dimethyl-6,6a-dihydro-3aH-furo[3,4-d][1,3]dioxol-4-yl]methyl (2S)-2-(tert-butoxycarbonylamino)-3-methyl-butanoate (50 mg, 0.1 mmol) was added isopropyl acetate (1.3 mL) to give a colorless solution under argon. This was cooled to 0° C. and then 5-6 N HCl in IPA (0.05 mL) was added dropwisely. After 1.5 h, TLC showed mainly SM. Then more 5-6 N HCl in IPA (0.05 mL) was added and this was put in the fridge overnight. The next day, some solids formed in the flask. This was filtered through a medium sintered glass frit and washed with Et₂O. Since the solids were hygroscopic, it was dissolved in MeOH and concentrated in vacuo. The previous mother liquor had contained added solids, which were filtered, dissolved in MeOH, and combined with the previous solution. Concentration in vacuo gave the product with an impurity. This was re-dissolved in EtOH and triturated with Et₂O. After stirring for a while, this mixture was filtered and the solids were dissolved in EtOH. Then more Et₂O was added and the solids were filtered after stirring. Finally, the solids were dissolved in MeOH, concentrated in vacuo, dissolved in water and lyophilized overnight to afford [(1S)-1-[[(2S,3S,4R,5R)-5-(2,4-dioxopyrimidin-1-yl)-2-fluoro-3,4-dihydroxy-tetrahydrofuran-2-yl]methoxycarbonyl]-2-methyl-propyl]ammonium chloride (EIDD-02971) (11 mg, 30%) as light yellow solids.

### Example 93. Synthesis of EIDD-2749-2′,3′,5′-Isoburyl triester (EIDD-02954)

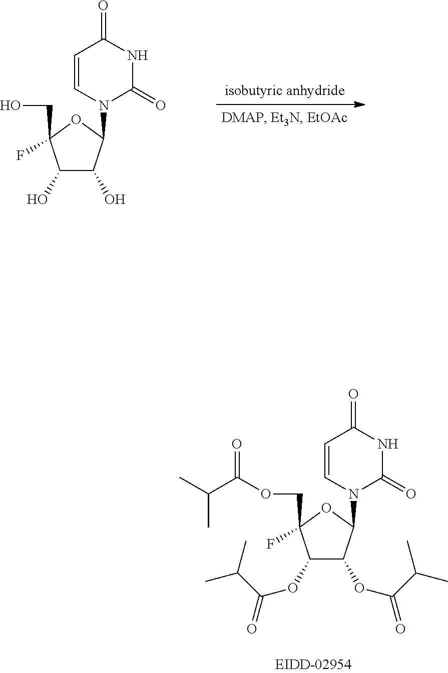 [SMILES: O=c1ccn([C@@H]2O[C@](F)(CO)[C@@H](O)[C@H]2O)c(=O)[nH]1 ; CC(C)C(=O)OC[C@@]1(F)O[C@@H](n2ccc(=O)[nH]c2=O)[C@H](OC(=O)C(C)C)[C@@H]1OC(=O)C(C)C]

To a 50 mL rbf charged with 1-[(2R,3R,4S,5S)-5-fluoro-3,4-dihydroxy-5-(hydroxymethyl)tetrahydrofuran-2-yl]pyrimidine-2,4-dione (68 mg, 0.26 mmol) and DMAP (6.3 mg, 0.05 mmol) was added EtOAc (2.6 mL) to give a suspension. This was vacuumed and charged with argon. Then Et₃N (0.18 mL, 1.3 mmol) was added. The flask was cooled to 0° C. and isobutyric anhydride (0.15 mL, 0.91 mmol) was added dropwisely. After 15 min, the resulting colorless solution was allowed to stir at room temperature. After 3.5 h, TLC showed no SM. Then water was added dropwisely. After stirring for 5 min, the reaction mixture was transferred into a separation funnel and more EtOAc was added. The organic layer was separated, dried (Na₂SO₄), filtered and concentrated in vacuo with Celite. The crude material was purified by ISCO column chromatography (24 g) eluting from 100% hexanes to 100% EtOAc to afford [(2S,3S,4R,5R)-5-(2,4-dioxopyrimidin-1-yl)-2-fluoro-3,4-bis(2-methylpropanoyloxy)tetrahydrofuran-2-yl]methyl 2-methylpropanoate (EIDD-02954) (0.1 g, 82%) as white solids.

¹H NMR (400 MHz, Methanol-d₄) δ 7.62 (d, J=8.0 Hz, 1H), 5.93-5.78 (m, 2H), 5.69 (d, J=7.9 Hz, 1H), 5.64 (dd, J=7.2, 2.1 Hz, 1H), 4.35 (dd, J=7.8, 3.5 Hz, 2H), 2.68-2.57 (m, 3H), 1.26-1.07 (m, 18H).

¹⁹F NMR (376 MHz, Methanol-d₄) δ −120.16 (dt, J=19.6, 7.8 Hz).

### Example 94. Synthesis of 4′-fluoro-4-thiouridine

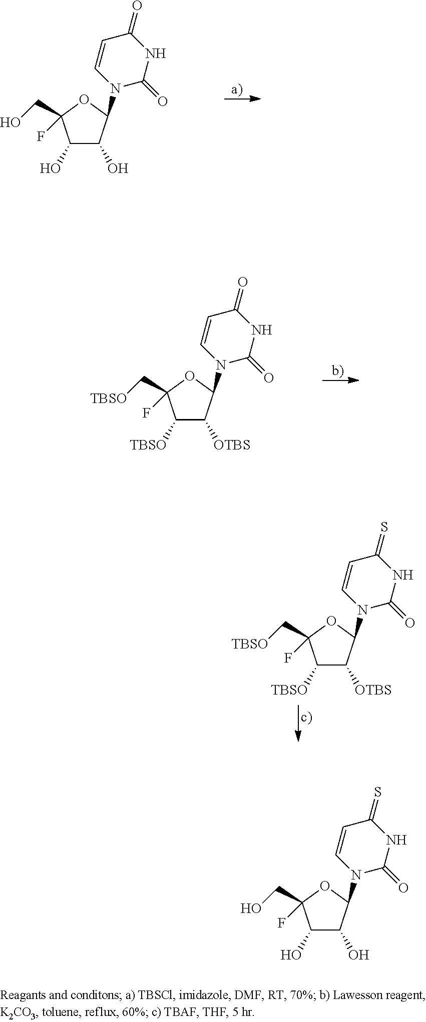 [SMILES: O=c1ccn([C@@H]2O[C@](F)(CO)[C@@H](O)[C@H]2O)c(=O)[nH]1 ; CC(C)(C)[Si](C)(C)OC[C@@]1(F)O[C@@H](n2ccc(=O)[nH]c2=O)[C@H](O[Si](C)(C)C(C)(C)C)[C@@H]1O[Si](C)(C)C(C)(C)C ; CC(C)(C)[Si](C)(C)OC[C@@]1(F)O[C@@H](n2ccc(=S)[nH]c2=O)[C@H](O[Si](C)(C)C(C)(C)C)[C@@H]1O[Si](C)(C)C(C)(C)C ; O=c1[nH]c(=S)ccn1[C@@H]1O[C@](F)(CO)[C@@H](O)[C@H]1O]

Preparation of 2′,3′,5′-tri-O-(t-butyldimethylsilyl)-4′-fluoro-uridine: To a solution of 4′-fluoro-uridine (500 mg, 1.9 mmol) in DMF (20 ml) taken in 100 ml RBF, TBDMSCl (1.2 gm, 7.6 mmol) and imidazole (650 mg, 9.5 mmol) were added under inert atmosphere at 0° C. and continued stirring at room temperature. After completion, the reaction mixture was concentrated under reduced pressure and the crude product was dissolved in dichloromethane and washed with saturated aq. NaHCO₃ followed by brine. The combined organic layers were dried over Na₂SO₄, filtered, and concentrated under reduced pressure. The residue was purified by chromatography. Product was obtained as colorless foam (yield 58%).

Preparation of 2′,3′,5′-tri-O-(t-butyldimethylsilyl)-4′-fluoro-4-thiouridine: To a solution of 2′,3′,5′-tri-O-(t-butyldimethylsilyl)-4′-fluoro-uridine (600 mg, 1 mmol) in anhydrous THF (20 ml), Lawesson's reagent (freshly purchased) (590 mg, 1.5 mmol) and potassium carbonate (29 mg, 0.2 mmol) were added and the reaction mixture was refluxed for 5 hr. After completion the reaction mixture was concentrated under reduced pressure and the crude product was purified by column chromatography. Product was obtained as colorless foam (yield 52%).

Preparation of 4′-fluoro-4-thiouridine: To a solution of 2′,3′,5′-tri-O-(t-butyldimethylsilyl)-4′-fluoro-4-thiouridine (250 mg, 0.41 mmol) in anhydrous tetrahydrofuran (5 ml), 1M solution of tetrabutylammonium fluoride (2 ml) was added and stirred at room temperature for 5 hr. After completion, the reaction mixture was concentrated under reduced pressure and the crude product was purified by silica gel column chromatography.

1H NMR 400 MHz, CD₃OD, δ 7.77 (1H, d, J=8 Hz), 6.06 (1H, d, J=4 Hz), 5.69 (1H, d, J=8 Hz), 4.42 (1H, dd, J=6.4 Hz, 20 Hz), 4.25 (1H, dd, 6.4 Hz, 2.4 Hz), 3.73 (2H, m); 19F NMR 376 MHz 6-123.57, (1F, dt, J=18.8 Hz, 3.7 Hz)

### Example 95. Synthesis of 1′-deutero-4′-fluorouridine

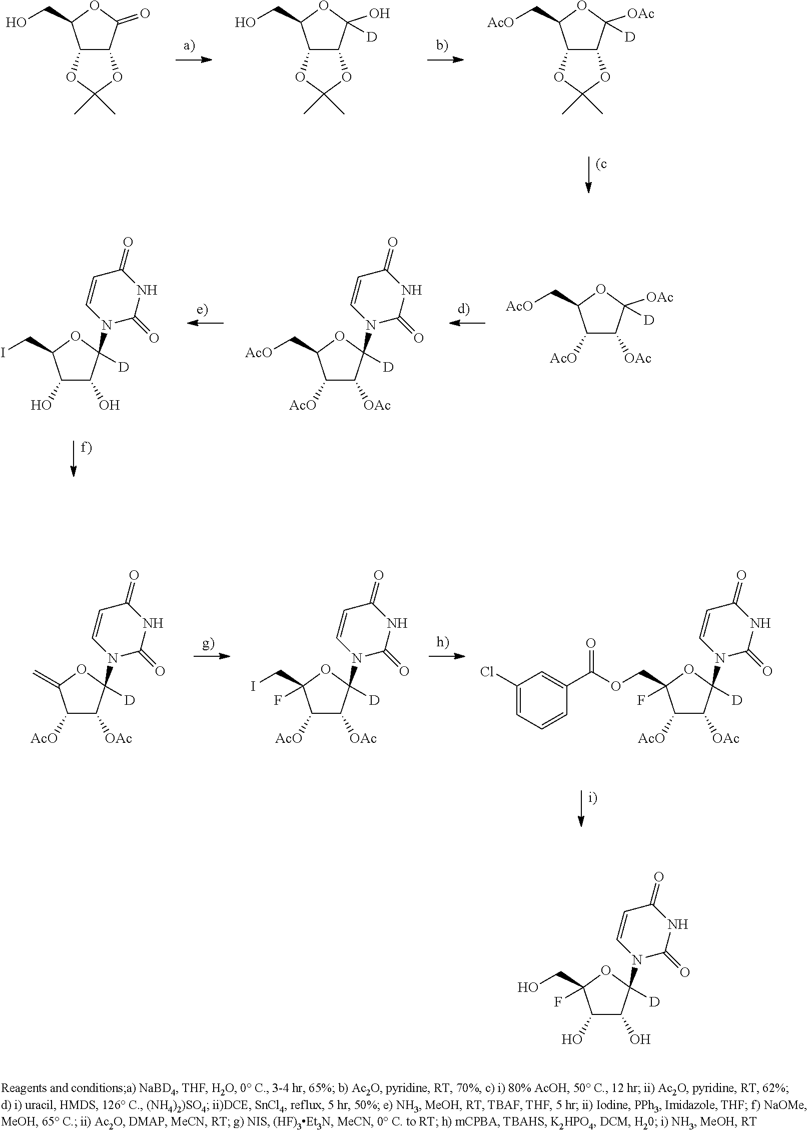

Preparation of 1-deutero-2,3-O-isopropylidene-D-ribofuranose: To a solution of 2,3-O-isopropylidene-D-ribonolactone (3 g, 16 mmol) in 9:1 (THF:H2O) (50 ml), taken in a 250 ml RBF, NaBD₄ (lg, 24 mmol) was added slowly in portions at 0° C. with continued stirring. After completion, the reaction mixture was quenched with acetone and stirred at room temperature for additional 30 min. The reaction mixture was diluted with excess ethyl acetate (100 ml) and washed with saturated aq. NH₄Cl followed by saturated aq. NaHCO₃ and brine. The combined organic layers were dried over Na₂SO₄, filtered, and concentrated under reduced pressure. The residue was purified by chromatography. The product was obtained as colorless oil (yield 65%).

Preparation of 1,5-di-O-acetyl-1-deutero-2,3-O-isopropylidene-D-ribofuranose: To a solution of 1-deutero-2,3-O-isopropylidene-D-ribofuranose (1.9 g, 10 mmol) in DCM (50 ml), acetic anhydride (2.4 ml, 25 mmol), trimethylamine (4.2 ml, 30 mmol), and DMAP (195 mg, 1.6 mmol) were added at 0° C. Stirring continued at room temperature. After completion, the reaction mixture was washed with saturated aq. NH₄Cl followed by saturated aq. NaHCO₃ (twice) and brine. The combined organic layers were dried over Na₂SO₄, filtered, and concentrated under reduced pressure. The residue was purified by chromatography. Product was obtained as colorless syrup (yield 70%).

Preparation of 1,2,3,5-tetra-O-acetyl-1-deutero-D-ribofuranose: 1,5-di-O-acetyl-1-deutero-2,3-O-isopropylidene-D-ribofuranose (2 g, 7.2 mmol) was dissolved in 80% acetic acid (50 ml) in 100 ml RBF and stirred at 50° C. for 12 hr. After completion, the reaction mixture was concentrated under reduced and co-evaporated with toluene twice. The crude product was dissolved in pyridine (20 ml). Acetic anhydride (1.7 ml, 18 mmol) and DMAP (122 mg, 1 mmol) were added at 0° C. and stirring continued at room temperature. After completion, the reaction mixture was concentrated under reduced pressure and the crude product was dissolved in dichloromethane and washed with 5% aq. HCl followed by saturated aq. NaHCO₃ and brine. The combined organic layers were dried over Na₂SO₄, filtered, and concentrated under reduced pressure. The residue was purified by chromatography. Product was obtained as syrup which crystallizes upon standing (yield 62% for 2 steps).

Preparation of 2′,3′,5′-tri-O-acetyl-1′-deutero-uridine: To a suspension of uracil (670 mg, 6 mmol) in HMDS (10 ml) in a 100 ml RBF, catalytic ammonium sulfate was added and refluxed at 126° C. under inert atmosphere for 12 hr. The reaction mixture cooled down and concentrated under reduced pressure. The residue was subjected to high vacuum and charged with anhydrous acetonitrile, compound 1,2,3,5-tetra-O-acetyl-1-deutero-D-ribofuranose (950 mg, 3 mmol) in acetonitrile and tin tetrachloride (350 μL, 3 mmol). The reaction mixture was refluxed under inert atmosphere for 5 hr. After completion, the reaction mixture was quenched with solid NaHCO₃ and Celite and stirred at room temperature for 30 min. A few drops of saturated aq. NaHCO₃ and continued stirring for 2-3 hr. The white precipitate formed was filtered and washed with DCM, the filtrate was concentrated under reduced pressure and purified by column chromatography. Product was obtained as colorless solid (yield 50%).

Preparation of 5′-deoxy-1′-deutero-5′-iodo-uridine: To a solution of 2′,3′,5′-tri-O-acetyl-1′-deutero-uridine (745 mg, 2 mmol) in methanol (10 ml), 7N ammonia in methanol was added and stirred at room temperature. After completion, the reaction mixture was concentrated under reduced pressure. The crude product was triturated with ethyl acetate and the resulting solid was taken in a 100 ml RBF and suspended in THF. Triphenylphosphine (786 mg, 3 mmol), imidazole (200 mg, 3 mmol) and iodine (600 mg, 2.3 mmol) were added and stirred at room temperature for 8 hr. After completion, the reaction mixture was concentrated under reduced pressure and the residue was stirred with isopropanol. The colorless solid formed was filtered and dried (yield 45%).

Preparation of compound 2′,3′-di-O-acetyl-1′-deutero-5′-deoxy-4′,5′-didehydrouridine: To a solution of 5′-deoxy-1′-deutero-5′-iodo-uridine (530 mg, 1.5 mmol) in methanol, sodium methoxide 25% by weight in methanol (325 μL) was added and stirred at 65° C. under inert atmosphere. After completion, the reaction mixture was concentrated under reduced pressure. The crude product was taken in MeCN (10 ml) and treated with acetic anhydride (425 μL, 4.5 mmol) and DMAP (20 mg, 0.15 mmol) and stirred at room temperature for 12 hr. After completion, the reaction mixture was quenched with saturated aq. NaHCO₃, diluted with DCM, washed with saturated aq. NaHCO₃ and brine. The combined organic layers were dried over Na₂SO₄, filtered, and concentrated under reduced pressure. The residue was purified by chromatography yielding product as colorless solid.

Preparation of compound 2′,3′-di-O-acetyl-1′-deutero-5′-deoxy-5′-iodo-4′-fluorouridine: To a solution of compound 2′,3′-di-O-acetyl-1′-deutero-5′-deoxy-4′,5′-didehydrouridine (460 mg, 2 mmol) in anhydrous acetonitrile (5 ml) in 50 ml RBF, triethylamine trihydrofluoride (162 μL, 1 mmol) and N-iodosuccinimide (2.6 mmol) were added at 0° C. After 60 min, the reaction mixture was slowly warmed to room temperature. After completion, the reaction mixture was concentrated under reduced pressure and purified by column chromatography.

Preparation of compound 2′,3′-di-O-acetyl-1′-deutero-5′-m-chlorobenzoate-4′-fluorouridine: To a solution of 2′,3′-di-O-acetyl-1′-deutero-5′-deoxy-5′-fluoro-4′-iodouridine (460 mg, 1 mmol) in 5:1 (DCM:H₂O) (50 ml) in a 100 ml RBF, tetrabutylammonium hydrogen sulfate (370 mg, 1.1 mmol) and potassium phosphate dibasic (260 mg, 1.5 mmol) were added, and the reaction mixture was cooled to 0° C. meta-chloroperbenzoic acid (860 mg, 4 mmol) was added slowly in portions and reaction mixture was allowed to warm to room temperature and vigorous stirring was continued for another 12 hr. After completion, the reaction mixture was quenched with aq. Na₂SO₃ and diluted with DCM (30 ml). The organic layer was separated and washed with saturated aq. NaHCO₃ and brine. The combined organic layers were dried over Na₂SO₄, filtered, and concentrated under reduced pressure. The residue was purified by chromatography.

Preparation of 1′-deutero-4′-fluorouridine: To a solution of 2′,3′-di-O-acetyl-1′-deutero-5′-m-chlorobenzoate-4′-fluorouridine (250 mg, 0.5 mmol) in methanol (10 ml), 7N ammonia in methanol (2 ml) was added and stirred at room temperature. After completion, the reaction mixture was concentrated under reduced pressure and purified by column chromatography.

### Example 96. Synthesis of 4′-fluoro-carbauridine

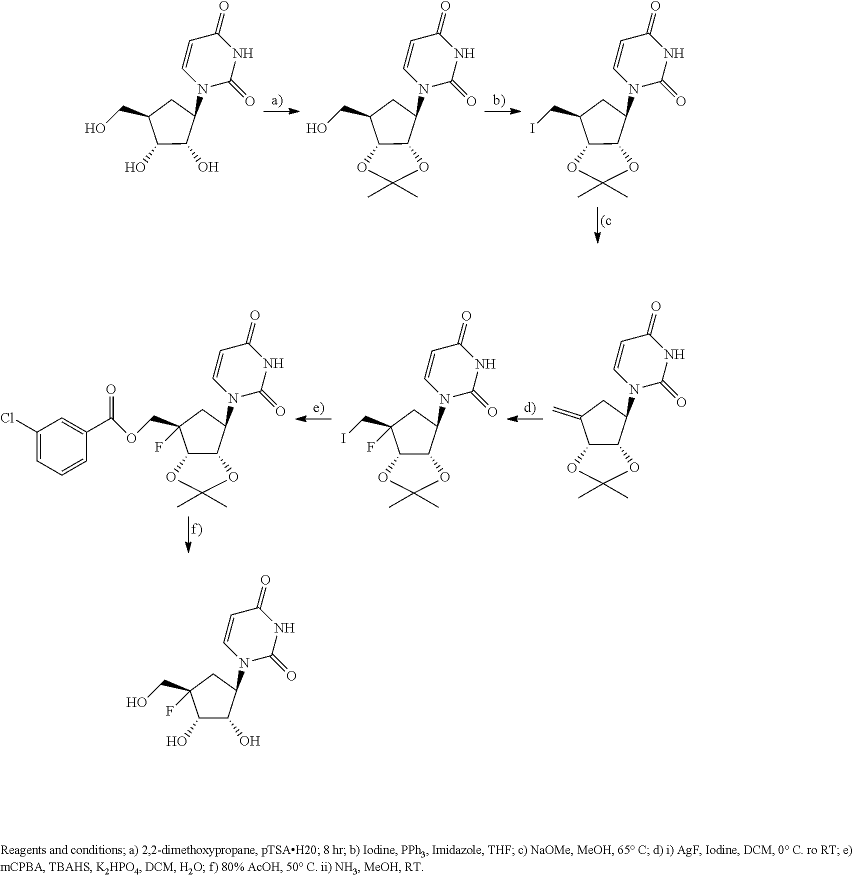

To a suspension of compound carbauridine (2.5 gm, 10 mmol) in anhydrous acetone (200 ml), 2,2-dimethoxypropane (1.2 ml, 10 mmol) and concentrated sulfuric acid (200 QL, 2 mmol) were added at 0° C. under inert atmosphere and stirring continued at room temperature. After completion, the reaction mixture was quenched with NaHCO₃, stirred for 30 min, and was filtered. The filtrate was concentrated under reduced pressure and the residue was purified by column chromatography.

To a solution of the above synthesized acetonide (1.4 gm, 5 mmol) in THF (100 ml) in a 250 ml RBF, triphenylphosphine (2 gm, 7.5 mmol), imidazole (500 mg, 7.5 mmol) and iodine (1.4 gm, 5.5 mmol) were added at 0° C. under inert atmosphere and stirred at room temperature for 8 hr. After completion, the reaction mixture was concentrated under reduced pressure and the residue was taken up in isopropanol (100 ml) and stirred at room temperature. The colorless solid formed was filtered and dried.

To a solution of the iodo compound synthesized above (1 gm, 2.5 mmol) in methanol, sodium methoxide 25% by weight in methanol (1.1 ml, 5 mmol) was added and stirred at 65° C. under an inert atmosphere. After completion, the reaction mixture was concentrated under reduced pressure. The crude product was dissolved in DCM (100 ml) and filtered through a Celite bed. The filtrate was concentrated under reduced pressure and the residue was purified by column chromatography.

To a solution of the alkene product obtained above (800 mg, 3 mmol) in anhydrous DCM (50 ml) in a 100 ml RBF, silver fluoride (950 mg, 7.5 mmol) was added followed by dropwise addition of iodine (1.5 gm, 6 mmol) in THF. After addition, the reaction mixture was slowly allowed to warm to room temperature and stirred for additional 30 min at room temperature. After completion, the reaction mixture was filtered through a Celite bed and the filtrate was washed with saturated aq. Na₂S₂O₃ followed by saturated aq. NaHCO₃ and brine. The combined organic layers were dried over Na₂SO₄, filtered, and concentrated under reduced pressure. The residue was purified by chromatography.

To a solution of the above compound (610 mg, 1.5 mmol) in 5:1 (DCM:H₂O) (50 ml) in a 100 ml RBF, tetrabutylammonium hydrogen sulfate (560 mg, 1.65 mmol) and potassium phosphate dibasic (400 mg, 2.3 mmol) were added. The reaction mixture was cooled to 0° C. m-Chloroperbenzoic acid (1.0 gm, 6 mmol) was added slowly in portions, and the reaction mixture was allowed to warm to room temperature. Vigorous stirring was continued for another 12 hr. After completion, the reaction mixture was quenched with aq. Na₂SO₃ and diluted with DCM (50 ml). The organic layer was separated and washed with saturated aq. NaHCO₃ and brine. The combined organic layers were dried over Na₂SO₄, filtered, and concentrated under reduced pressure. The residue was purified by chromatography.

The above compound (450 mg, 1 mmol) was taken up in 80% acetic acid (20 ml) and stirred at 50° C. for 12 hr. After completion, the reaction mixture was concentrated under reduced pressure and co-evaporated twice with anhydrous toluene. The residue was taken in methanol (20 ml) and treated with 7N ammonia in methanol (2 ml) and stirred at room temperature. After completion, the reaction mixture was concentrated under reduced pressure and purified by column chromatography to provide the final desired product.

### Example 97. Synthesis of 4′-fluoro-2-thiouridine

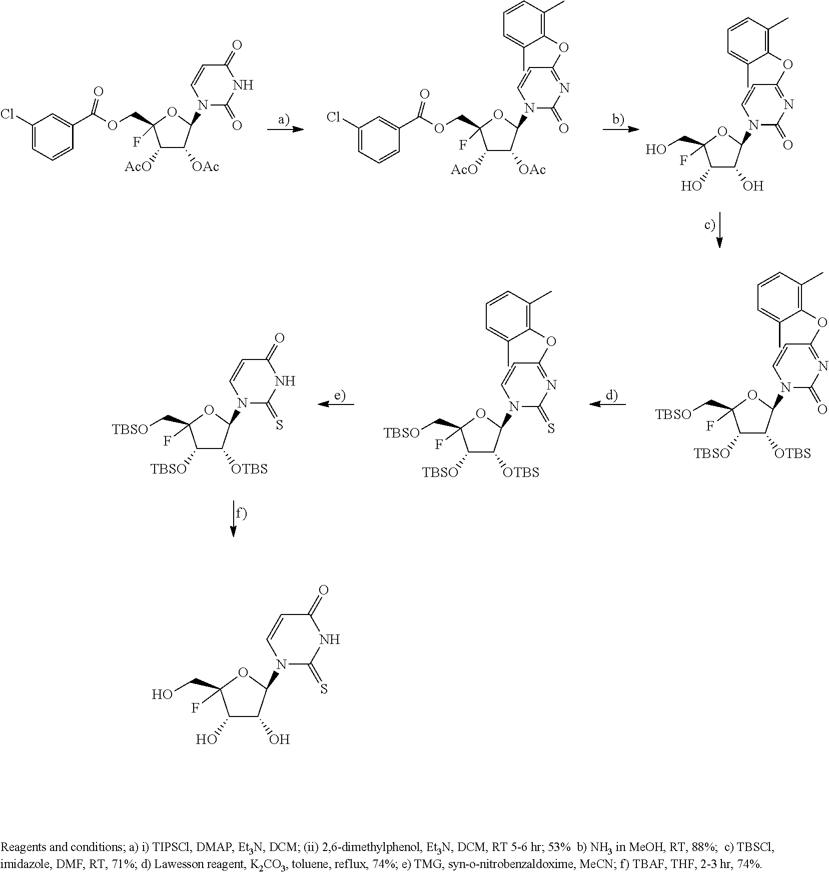

Preparation of 4-O-(2,6-dimethylphenyl)-2′,3′-di-O-acetyl-5′-O-(4-chlorobenzoyl)-4′-fluorouridine: 2′,3′-di-O-acetyl-5′-O-(4-chlorobenzoyl)-4′-fluorouridine (1 gm, 2 mmol) was dissolved in anhydrous dicholoromethane (30 ml) in 100 ml RBF. Et₃N (542 μL, 3.75 mmol), 2,4,6-triisopropylbenzensulfonyl chloride (690 mg, 2.26 mmol), and 4-(dimethylamino)pyridine (62 mg, 0.5 mmol) were added at 0° C. under an inert atmosphere with continued stirring at room temperature. After completion of the reaction, 2,6-dimethylphenol (300 mg, 2.45 mmol), Et₃N (3.45 mL, 25 mmol), and 1,4-diazabicyclo[2,2,2]octane (23 mg, 0.2 mmol) were added at 0° C. under an inert atmosphere with continued stirring at room temperature for 3-4 hr. The reaction mixture was diluted with dichloromethane (30 ml) and washed once with saturated NaHCO₃ (aqueous) and twice with brine. The combined organic extracts were dried over Na₂SO₄, filtered, and concentrated under reduced pressure. The residue was purified by column chromatography. Product was obtained as colorless solid (yield 53%).

Preparation of 4-O-(2,6-Dimethylphenyl)-4′-fluorouridine: To a solution of 4-O-(2,6-Dimethylphenyl)-2′,3′-di-O-acetyl-5′-O-(4-chlorobenzoyl)-4′-fluorouridine (600 mg) in anhydrous methanol (6 ml) in 25 ml RBF, 1 ml of 7N ammonia in methanol was added and stirred at room temperature for 8 hr. After completion, the reaction mixture was concentrated under reduced pressure and the crude product was obtained as colorless solid (yield 88%).

Preparation of 4-O-(2,6-dimethylphenyl)-2′,3′,5′-tri-O-(t-butyldimethylsilyl)-4′-fluorouridine: To a solution of 4-O-(2,6-dimethylphenyl)-4′-fluorouridine (720 mg) in anhydrous DMF (10 ml) in a 50 ml RBF, tert-butyldimethylsilyl chloride (1185 mg, 7.8 mmol) and imidazole (670 mg, 9.8 mmol) were added at 0° C. under an inert atmosphere with continued stirring at room temperature for 12 hr. After completion the reaction mixture was concentrated under reduced pressure and the crude product was taken up in DCM and washed with saturated aq.NaHCO₃ and with brine. The combined organic extracts were dried over Na₂SO₄, filtered, and concentrated under reduced pressure. The residue was purified by column chromatography to get colorless foam (yield 71%)

Preparation of 4-O-(2,6-dimethylphenyl)-2′,3′,5′-tri-O-(t-butyldimethylsilyl)-4′-fluoro-2-thiouridine: To a solution of 4-O-(2,6-dimethylphenyl)-2′,3′,5′-tri-O-(t-butyldimethylsilyl)-4′-fluorouridine (750 mg, 1 mmol) in anhydrous toluene (20 ml), Lawesson's reagent (freshly purchased) (590 mg, 1.5 mmol) and potassium carbonate (29 mg, 0.2 mmol) were added and the reaction mixture was refluxed for 8 hr. After completion, the reaction mixture was concentrated under reduced pressure and the crude product was purified by column chromatography. Product was obtained as colorless foam (yield 74%).

Preparation of 2′,3′,5′-tri-O-(t-butyldimethylsilyl)-4′-fluoro-2-thiouridine: To a solution of 4-O-(2,6-dimethylphenyl)-2′,3′,5′-tri-O-(t-butyldimethylsilyl)-4′-fluoro-2-thiouridine (500 mg, 0.68 mmol) in acetonitrile (10 ml), 1,1,3,3-tetramethylguanidine (260 μL, 2 mmol) and syn-o-nitrobenzaldoxime (343 mg, 2 mmol) were added and stirred at room temperature for 5 hr. After completion, the mixture was concentrated under reduced pressure. The crude product was dissolved in dichloromethane and washed with saturated aq. NaHCO₃ and with brine. The combined organic layers were dried over Na₂SO₄, filtered, and concentrated under reduced pressure. The residue was purified by chromatography. Product was obtained as colorless foam (yield 67%).

Preparation of 4′-fluoro-2-thiouridine: To a solution of 2′,3′,5′-tri-O-(t-butyldimethylsilyl)-4′-fluoro-2-thiouridine (270 mg, 0.43 mmol) in anhydrous tetrahydrofuran (5 ml), 1M solution of tetrabutylammonium fluoride (2 ml) was added and stirred at room temperature for 5 hr. After completion, the reaction mixture was concentrated under reduced pressure and the crude product was purified by silica gel column chromatography. Product was obtained as off white solid (yield 74%).

1H NMR 400 MHz, CD₃OD, δ 8.11 (1H, d, J=8 Hz), 6.84 (1H, s), 5.94 (1H, d, J=8 Hz), 4.26 (2H, m), 3.78 (2H, m); 13C NMR 100 MHz δ 176.49, 159.90, 140.78, 119.46, 117.16, 107.34, 95.08, 72.83, 68.59, 59.80; 19F NMR 376 MHz δ −122.77, (1F, d, J=18.8 Hz); LCMS: [M⁺]+279.0.

### Example 98. Protocol for Determining Plasma Stability

Test article, e.g., EIDD-2749 prodrug, was incubated in triplicate at 1.00 μM in pooled mixed gender human plasma (BioIVT, K₂EDTA), in pooled male CD-1 mouse plasma (BioIVT, K₂EDTA, gender pool, e.g., Lot MSE463495), in pooled male Sprague-Dawley rat plasma (BioIVT, lithium heparin). Incubations were performed in 13×100 mm glass culture tubes. Samples were placed in a water bath shaker set at 37° C. and shaken at 150 rpm. Procaine, Benfluorex or Enalapril (1 μM, each) were run in parallel as a positive controls for human, mouse or rat plasma activity, respectively.

Aliquots of 100 μL were taken at the following time-points: 0, 5, 15, 30, 60, and 120 minutes. These aliquots were mixed with 400 μL of 100% acetonitrile in 1.7-mL conical polypropylene microcentrifuge tubes. Samples were vortexed for about 10 seconds and then clarified by centrifugation (2 minutes at 15,000 g). Supernatants were analyzed by LC-MS/MS.

HPLC separation was performed on an Agilent 1200 system (Agilent Technologies, Santa Clara, CA, USA) equipped with a column oven, UV lamp, and binary pump. A Thermo Hypercarb PGC (150×4.6 mm, 5 μm) column (ThermoFisher, Waltham, MA USA) was used for the separation. Mobile Phase A consisted of 100 mM Ammonium Bicarbonate buffer in HPLC grade Water (pH 10) and Mobile phase B consisted of neat acetonitrile. A gradient 0-85% of B was run for 3 minutes followed by 0% B for 4 minutes was used for the separation. Mass Spectrometry analysis was performed on a Triple Quad 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA).

Analyte concentrations were calculated based on standard curve. Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln[μM] vs. time). Half-life (t_{1/2}) (min)=−0.693/(slope).

### Example 99. Protocol for Determining Liver Microsome Stability

Test article was incubated in triplicate at 1.00 μM in 100 mM phosphate buffer (pH 7.4), Phase I cofactors (NADPH Regenerating System) and 0.5 mg (total protein) from pooled gender human liver microsomes (BioIVT), pooled male CD-1 mouse liver microsomes (XenoTech) or pooled male Sprague-Dawley rat liver microsomes (BioIVT). Incubations were performed in 13×100 mm glass culture tubes. Samples were placed in a water bath shaker set at 37° C. and shaken at 150 rpm. Verapamil (1 μM) was run in parallel as a positive control.

In some studies, test article, e.g., EIDD-2749 prodrug, was incubated in triplicate at 1.00 μM in 100 mM phosphate buffer containing 3 mM MgCl₂ and 1 mM EDTA, 1 mM NADPH (or 2% (w/v) sodium bicarbonate in water for no-NADPH controls) and 0.5 mg of total protein from a pool of CD-1 female liver microsomes (BioIVT, Lot DVO, 880 pmol/mg protein/min for the 6-β-hydroxylation of testosterone=CYP3A4 probe).

In some studies, test article, e.g., EIDD-2749 prodrug, was incubated in triplicate at 1.00 μM in 100 mM phosphate buffer containing 3 mM MgCl₂ and 1 mM EDTA, 1 mM NADPH (or 2% (w/v) sodium bicarbonate in water for no-NADPH controls) and 0.3 mg of total protein from a pool of CD-1 male intestinal microsomes (BioIVT, Lot 2210211, 1420 pmol/mg protein/min for the 6-β-hydroxylation of testosterone=CYP3A4 probe). Positive controls (1 μM verapamil and 1 mm 4-methylumbellifurone) to access the test systems competencies and to compare the metabolic efficiency and activities of the intestinal microsomes to the hepatic microsomes).

HPLC separation was performed on an Agilent 1200 system (Agilent Technologies, Santa Clara, CA, USA) equipped with a column oven, UV lamp, and binary pump. A Thermo Hypercarb PGC (150×4.6 mm, 5 μm) column (ThermoFisher, Waltham, MA USA) was used for the separation. Mobile Phase A consisted of 100 mM Ammonium Bicarbonate buffer in HPLC grade Water (pH 10) and Mobile phase B consisted of neat acetonitrile. A gradient 0-85% of B was run for 3 minutes followed by 0% B for 4 minutes were used for the separation. Mass Spectrometry analysis was performed on a Triple Quad 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA).

Analyte concentrations were calculated based on Standard curve. Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln[μM] vs. time). Half-life (t_{1/2}) (min)=−0.693/(slope).

### Example 100. Protocol for Determining pH Stability

Test article in methanol, water, 0.1N HCl, PBS or pH9 buffer were placed in the HPLC autosampler set at 25° C. or 4° C. Samples were injected on the LC-MS/MS at times: 0, 1, 2, 3, 4, 6 and 24 hours. HPLC separation was performed on an Agilent 1200 system (Agilent Technologies, Santa Clara, CA, USA) equipped with a column oven, UV lamp, and binary pump. A Thermo Hypercarb PGC (100×4.6 mm, 5 μm) column (ThermoFisher, Waltham, MA USA) was used for the separation. Mobile Phase A consisted of 25 mM ammonium bicarbonate buffer in HPLC grade water (pH 9.4) and Mobile phase B consisted of neat acetonitrile. Initial mobile phase conditions of 5% B were held for a minute. A gradient 5-60% of B was run for next 7 minutes, followed by re-equilibration of the column, was used. Mass Spectrometry analysis was performed on a QTRAP 5500 Mass Spectrometer (AB Sciex, Framingham, MA, USA) using Negative Mode Electrospray Ionization (ESI) in Multiple Reaction Monitoring (MRM) Mode and UV at 260 nm. Data analysis was performed using Analyst Software (AB Sciex, Framingham, MA, USA). Stability was determined by the % UV peak area change from the time-zero samples.

### Example 101. Stability of EIDD-2749 in Solvents and Buffers

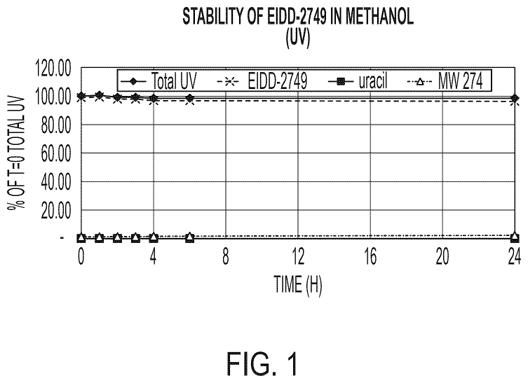

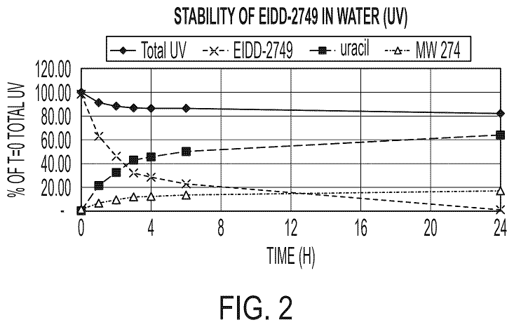

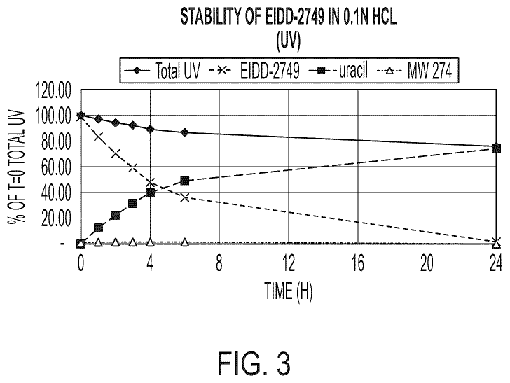

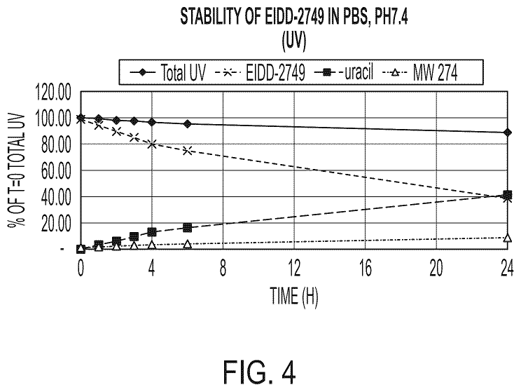

The stability of EIDD-2749 in solvents and acidic, neutral, and basic buffers is shown in FIGS. 1-5.

### Example 102. Stability of EIDD-2749 Prodrugs in Plasma and Liver Microsomes

| species | matrix | drug (1.00 μM, each) | half-life (min) | max [2749], μM |
|---|---|---|---|---|
| human | plasma | EIDD-2749 (nuc) | 26 | NA |
| | | EIDD-2838 (cycloSal) | 12 | 0.0521 |
| | | EIDD-2947 (mono-ester) | 2 | 0.653 |
| | | EIDD-2954 (tri-ester) | 2 | 0.0390 |
| | | EIDD-2971 (valine) | 81 | 0.0152 |
| | liver microsomes | EIDD-2749 (nuc) | 22 | NA |
| | | EIDD-2838 (cycloSal) | 7 | 0.130 |
| | | EIDD-2947 (mono-ester) | 44 | 0.209 |
| | | EIDD-2954 (tri-ester) | <1 | 0.342 |
| | | EIDD-2971 (valine) | 38 | 0.179 |
| mouse | plasma | EIDD-2749 (nuc) | 37 | NA |
| | | EIDD-2838 (cycloSal) | 1 | 0.0216 |
| | | EIDD-2947 (mono-ester) | 1 | 1.18 |
| | | EIDD-2954 (tri-ester) | 1 | 0.716 |
| | | EIDD-2971 (valine) | 59 | 0.267 |
| | liver microsomes | EIDD-2749 (nuc) | 27 | NA |
| | | EIDD-2838 (cycloSal) | 7 | 0.0347 |
| | | EIDD-2947 (mono-ester) | 1 | 0.602 |
| | | EIDD-2954 (tri-ester) | <1 | 0.256 |
| | | EIDD-2971 (valine) | 11 | 0.305 |
| rat | plasma | EIDD-2749 (nuc) | 22 | NA |
| | | EIDD-2838 (cycloSal) | <1 | 0.0341 |
| | | EIDD-2947 (mono-ester) | 9 | 0.518 |
| | | EIDD-2954 (tri-ester) | 1 | 0.142 |
| | | EIDD-2971 (valine) | 50 | 0.155 |
| | liver microsomes | EIDD-2749 (nuc) | 25 | NA |
| | | EIDD-2838 (cycloSal) | 8 | 0.218 |
| | | EIDD-2947 (mono-ester) | 10 | 0.423 |
| | | EIDD-2954 (tri-ester) | <1 | 0.149 |
| | | EIDD-2971 (valine) | 33 | 0.203 |

### Example 103. EIDD-02991 Concentrations in Huh-7 Cells

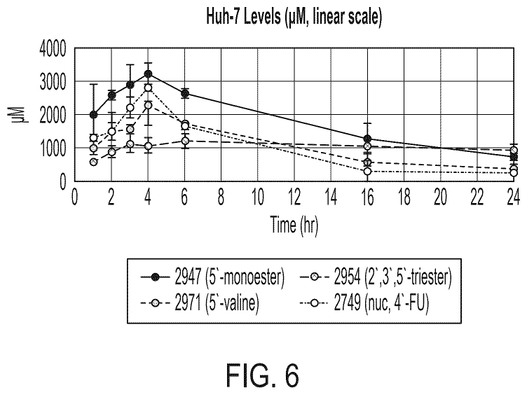

EIDD-02991 concentrations in Huh-7 cells incubated with EIDD-2749, EIDD-02947, EIDD-02954, or EIDD-02971 are shown in FIG. 6.

### Example 104. EIDD-02991 Concentrations in Vero Cells

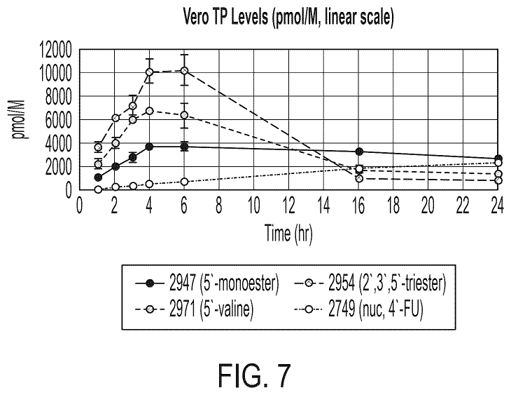

EIDD-02991 concentrations in Vero cells incubated with EIDD-2749, EIDD-02947, EIDD-02954, or EIDD-02971 are shown in FIG. 7.

### Example 105. Mouse PK Protocol

Female ICR (CD-1) mice (from Envigo) between the ages of 7 to 8 weeks were acclimated to their environment for at least three days prior to dosing. Mice were weighed at least once before dosing to determine the dosing volume.

Test article was dissolved in sterile saline at 1 mg/mL for IP dosing. For oral dosing, test article was resuspended in 10 mM trisodium citrate/0.5% Tween 80/Water. For IP dosing mice were dosed with a 10 mL/kg dose volume and mice dosed PO were dosed with a 10 mL/kg dose volume.

Blood samples collected from mice dosed by oral gavage were collected pre-dose, 0.25, 0.50, 1, 2, 3, 4, 8, and 24 hours post-dose. Blood samples collected from mice dosed by intraperitoneal injection were collected pre-dose, 0.08, 0.25, 0.50, 1, 2, 3, 4, and 8 hours post-dose. Blood samples were collected by reto-orbital bleeding under isoflurane anesthesia into lithium-heparin microtainer tubes, centrifuged at 2000×g for 10 min at 5° C., and the plasmas were transferred into fresh tubes and stored at −80° C. before processing for quantitation by LC-MS/MS.

50 μL aliquots of mouse plasma were extracted with 950 μL of acetonitrile that included EIDD-2216 as an Internal Standard. Samples were clarified by centrifugation at 20,000×g at 4° C. for 10 min. The clarified supernatants were transferred to HPLC vials for analysis.

Samples were maintained at 4° C. in a Leap Pal Autosampler (CTC Analytics AG, Zwingen, Switzerland). HPLC separation was performed on an Agilent 1200 system (Agilent Technologies, Santa Clara, CA, USA) equipped with a column oven, UV lamp, and binary pump. An Agilent SB-Phenyl (150×4.6 mm, 5 μm) column (Agilent technologies, Santa Clara, CA, USA) was used for the separation. Mobile Phase A consisted of 100 mM Ammonium Formate buffer in HPLC grade Water and Mobile phase B consisted of pure acetonitrile. An initial 1 minute isocratic step was used at 5% Mobile Phase B followed by a 1.5 minute gradient to 100% Mobile Phase B, which was held for 1.5 minutes before returning to starting conditions for 1.5 minutes. Mass Spectrometry analysis was performed on an QTRAP 5500 Mass Spectrometer (AB Sciex, Farmingham, MA, USA) using Negative Mode Electrospray Ionization (ESI) in Multiple Reaction Monitoring (MRM) Mode. Data analysis was performed using Analyst Software (AB Sciex, Farmingham, MA, USA).

PK parameters are calculated using the Phoenix WinNonLin 6.4 (Build 6.4.0.768) Non-compartmental analysis tool (Certara, Princeton, NJ, USA). Bioavailability is calculated by comparing the exposure (AUCinf) after oral dosing with the exposure after intraperitoneal dosing.

### Example 106. EIDD-2749 Mouse PK Results

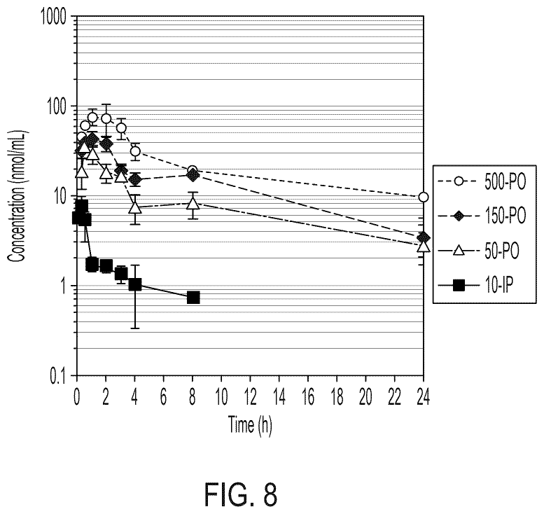

Graphs for EIDD-2749 CD-1 mouse PK are shown in FIG. 8. The PK parameters are shown in the table below.

| | | | | | Cₘₐₓ/Dose | AUC-INF/Dose | | |
|---|---|---|---|---|---|---|---|---|
| Dose | tₘₐₓ | Cₘₐₓ | C_{24 h} | AUC-INF | (kg*nmol/ | (h*kg*nmol/ | t_{1/2} | |
| (mg/kg) | (h) | (nmol/mL) | (nmol/mL) | (h*nmol/mL) | mL/mmol) | mL/mmol) | (h) | Bioavailability* |
| 10-IP | 0.25 | 7.7 ± 2.3 | 0.75 ± 0.1 | 19.6 | 202.1 | 514.5 | 6.3 | N/A |
| 50-PO | 0.5 | 35.2 ± 9.7 | 2.8 ± 1.1 | 232.3 | 184.3 | 1216.0 | 8.3 | 236% |
| 150-PO | 1 | 43.6 ± 2.8 | 3.4 ± 1.3 | 392.3 | 76.2 | 685.8 | 8.5 | 133% |
| 500-PO | 1 | 76.0 ± 15.3 | 9.8 ± 4.2 | 755.2 | 39.9 | 396.0 | 12.9 | 77.0% |

### Example 107. Mouse Tolerability Protocol

AG129 mice between the ages of 6 to 10 weeks were acclimated to their environment for at least three days prior to dosing. Mice were weighed daily as well as monitored for morbidity and mortality daily. For oral dosing, test article was resuspended in 10 mM trisodium citrate/0.5% Tween 80/Water. Mice dosed PO were dosed with a 10 mL/kg dose volume. Mice were dosed PO at 10, 30, and 100 mg/kg QD for 10 days.

### Example 108. Tolerability of EIDD-2749 in AG129 Mice

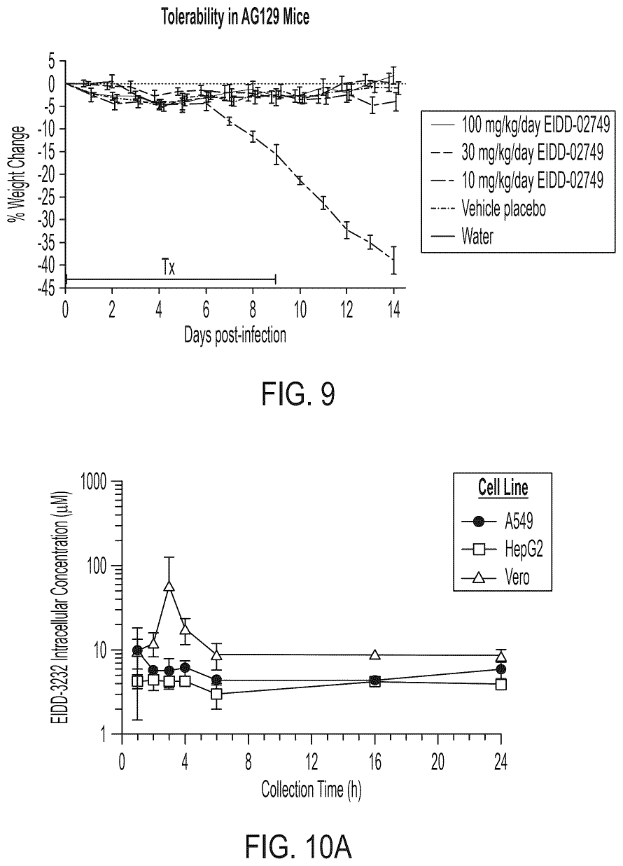

Results for tolerability of EIDD-2749 in AG129 mice arer shown in FIG. 9.

### Example 109. Synthesis of EIDD-3232 Precursor: isopropyl ((S)-(((2S,3S,4R,5R)-3,4-bis(((benzyloxy)carbonyl)oxy)-5-(2,4-dioxo-3,4-dihydropyrimidin-1(2H)-yl)-2-fluorotetrahydrofuran-2-yl)methoxy)(phenoxy)phosphoryl)-L-alaninate

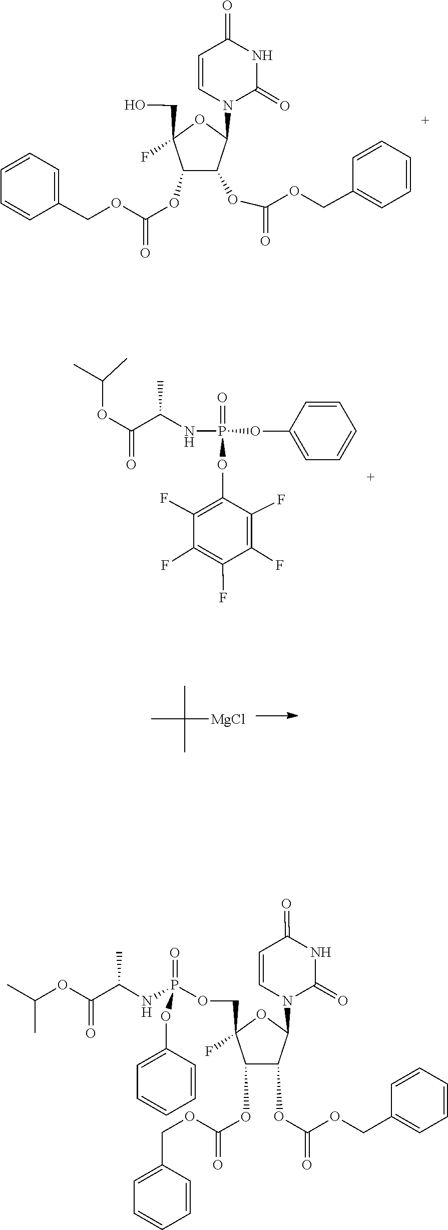

Nucleoside intermediate, dibenzyl ((2S,3S,4R,5R)-5-(2,4-dioxo-3,4-dihydropyrimidin-1(2H)-yl)-2-fluoro-2-(hydroxymethyl)tetrahydrofuran-3,4-diyl) bis(carbonate) (1.10 g, 2.07 mmol) was vacuum oven-dried at 50° C. for 16 h, and then was loaded into an oven-dried 100 mL rb and dissolved in anhydrous THF (15 mL). The solution was cooled to 0° C. and then treated dropwise with tert-butylmagnesium chloride (2.59 mL, 2.59 mmol) over a 5 min period. The mixture was stirred at cold temp for 20 minutes and then allowed to warm to room temperature and stirred an additional 1 h. The mixture was cooled back to OC and treated dropwise with an anhydrous THF (10 mL) solution of isopropyl ((S)-(perfluorophenoxy)(phenoxy)phosphoryl)-L-alaninate(1.17 g, 2.59 mmol) over a 5 min period. The mixture was allowed to stir overnight (16 h) at room temperature. The next day thin layer chromatography (10% methanol in DCM) showed mostly product as a faster moving component with some remaining starting material. The mixture was treated with saturated ammonium chloride solution (50 mL) and extrated with ethyl acetate (150 mL). The organic phase was dried over sodium sulfate, concentrated and then purified by column chromatography over silica gel using the CombiFlash equipped with an 80 g silica gel column using a methanol/DCM gradient. A second purification by column chromatography was carried out to yield the desired product, isopropyl ((S)-(((2S,3S,4R,5R)-3,4-bis(((benzyloxy)carbonyl)oxy)-5-(2,4-dioxo-3,4-dihydropyrimidin-1(2H)-yl)-2-fluorotetrahydrofuran-2-yl)methoxy)(phenoxy)phosphoryl)-L-alaninate (660 mg, 0.8253 mmol, 39.8% yield) as a white foam. ¹H NMR (400 MHz, Chloroform-d) δ 8.46 (s, 1H), 7.40-7.29 (m, 9H), 7.28 (d, J=1.5 Hz, 1H), 7.26 (s, OH), 7.20-7.09 (m, 4H), 5.82 (dd, J=17.5, 7.1 Hz, 1H), 5.78 (d, J=2.5 Hz, 1H), 5.69 (dd, J=8.0, 2.3 Hz, 1H), 5.53 (dd, J=7.0, 2.5 Hz, 1H), 5.18 (q, J=12.0 Hz, 2H), 5.09 (d, J=2.3 Hz, 2H), 5.00 (p, J=6.3 Hz, 1H), 4.34 (dt, J=7.6, 5.2 Hz, 2H), 4.07 (t, J=10.0 Hz, 1H), 4.04-3.93 (m, 1H), 1.37 (d, J=6.9 Hz, 3H), 1.21 (dd, J=6.3, 3.9 Hz, 6H). ¹⁹F NMR (376 MHz, Chloroform-d) δ −119.67 (dt, J=17.3, 5.5 Hz). ³¹P NMR (162 MHz, Chloroform-d) δ 1.95.

### Example 110. Synthesis of EIDD-3232 (isopropyl ((S)-(((2S,3S,4R,5R)-5-(2,4-dioxo-3,4-dihydropyrimidin-1(2H)-yl)-2-fluoro-3,4-dihydroxytetrahydrofuran-2-yl)methoxy)(phenoxy)phosphoryl)-L-alaninate)

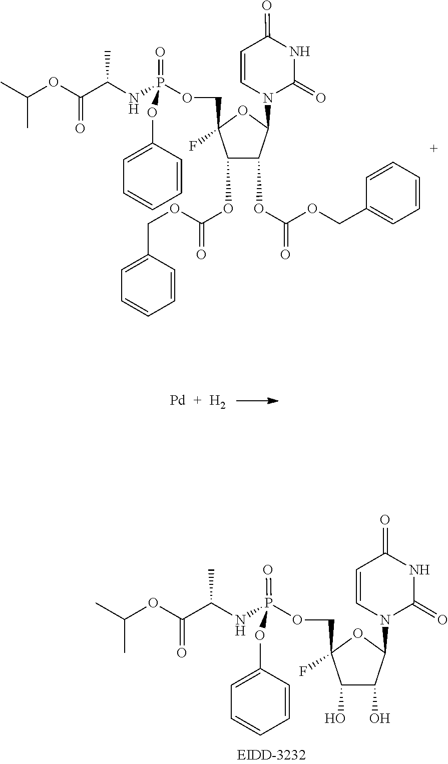

In a two necked round-bottomed flask equipped with gas inlets, a solution of isopropyl ((S)-(((2S,3S,4R,5R)-3,4-bis(((benzyloxy)carbonyl)oxy)-5-(2,4-dioxo-3,4-dihydropyrimidin-1(2H)-yl)-2-fluorotetrahydrofuran-2-yl)methoxy)(phenoxy)phosphoryl)-L-alaninate (660.00 mg, 0.83 mmol) in ethanol (10 mL) at 0° C. was degassed and treated with 10% palladium on carbon (100 mgs). The mixture was degassed and allowed to stir under hydrogen at atmospheric pressure at 0° C. for 30 min after which time TLC (10% to 20% methanol in DCM) indicated complete reaction. The mixture was filtered through a pad of Celite and then concentrated to dryness. The resulting white solid was further dried under high vacuum overnight to yield isopropyl ((S)-(((2S,3S,4R,5R)-5-(2,4-dioxo-3,4-dihydropyrimidin-1(2H)-yl)-2-fluoro-3,4-dihydroxytetrahydrofuran-2-yl)methoxy)(phenoxy)phosphoryl)-L-alaninate, EIDD-3232 (399 mg, 88%). ¹H NMR (400 MHz, Methanol-d₄) δ 7.54 (d, J=8.1 Hz, 1H), 7.36 (dd, J=8.6, 7.3 Hz, 2H), 7.25 (dt, J=8.6, 1.2 Hz, 2H), 7.22-7.16 (m, 1H), 6.00 (d, J=2.3 Hz, 1H), 5.64 (d, J=8.0 Hz, 1H), 4.97 (h, J=6.2 Hz, 1H), 4.51 (dd, J=18.0, 6.8 Hz, 1H), 4.34 (dd, J=6.8, 2.4 Hz, 1H), 4.32-4.26 (m, 2H), 3.92 (dq, J=10.0, 7.1 Hz, 1H), 1.37-1.32 (m, 3H), 1.23 (dd, J=6.3, 3.1 Hz, 6H). ¹³C NMR (101 MHz, Methanol-d₄) δ 172.95 (d, J=5.4 Hz), 164.48, 150.61 (d, J=6.9 Hz), 150.31, 142.04, 129.41, 124.86, 119.98 (d, J=4.9 Hz), 115.21 (dd, J=230.8, 9.9 Hz), 101.94, 94.30, 71.22, 69.70 (d, J=20.7 Hz), 68.81, 63.90 (dd, J=45.2, 5.0 Hz), 50.20, 20.57, 20.49, 19.10 (d, J=6.5 Hz). ¹⁹F NMR (376 MHz, Methanol-d₄) δ −123.96 (dt, J=18.1, 6.7 Hz). ³¹P NMR (162 MHz, cd₃od) δ 3.43. ESI-MS: m/z 530 ([M−H]⁻).

### Example 111. Synthesis of EIDD-3321 (N-((((2S,3S,4R,5R)-5-(2,4-dioxo-3,4-dihydropyrimidin-1(2H)-yl)-2-fluoro-3,4-dihydroxytetrahydrofuran-2-yl)methoxy)(hydroxy)phosphoryl)-N-methyl-L-alanine)

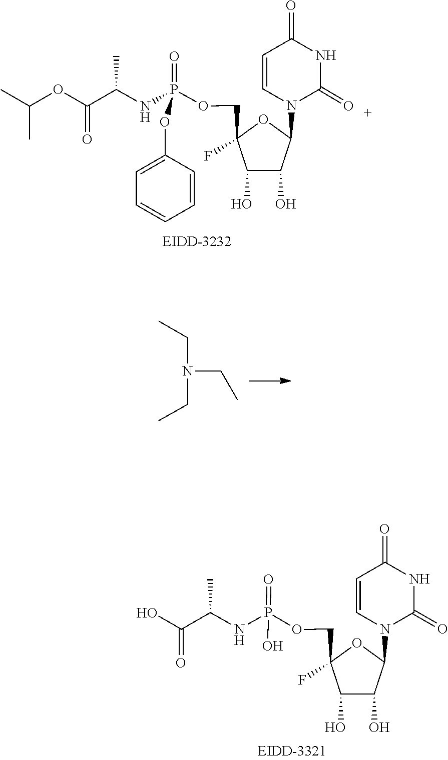

In a 100 mL round-bottomed flask, a solution of EIDD-3232, isopropyl ((S)-(((2S,3S,4R,5R)-5-(2,4-dioxo-3,4-dihydropyrimidin-1(2H)-yl)-2-fluoro-3,4-dihydroxytetrahydrofuran-2-yl)methoxy)(phenoxy)phosphoryl)-L-alaninat (248.00 mg, 0.47 mmol) was suspended in a 1:1 mixture of triethylamine:water (40 mL) and heated to 35° C. with vigorous stirring for 15 min. After cooling to room temperature, the mixture was concentrated and the resulting gum co-evaporated with water (3×10 mL). The residue was then purified by DEAE-sepahadex chromatography (1.5 cm×12 cm) using a mobile phase gradient 0 to 700 mM ammonium bicarbonate in 10% aqueous ethanol (1 L). Fractions were analyzed by TLC (7:2:1 isopropanol:ammonium hydroxide:water). Product containing fractions were concentrated followed by co-evaporation with water (3×20 mL). The resulting gum was dissolved in water (8 mL), frozen, and lyophilized to give EIDD-3321, isopropyl ((S)-(((2S,3S,4R,5R)-5-(2,4-dioxo-3,4-dihydropyrimidin-1(2H)-yl)-2-fluoro-3,4-dihydroxytetrahydrofuran-2-yl)methoxy)(phenoxy)phosphoryl)-L-alaninate (22 mg, 11% yield) as a white solid (93% purity area percent). ¹H NMR (400 MHz, D₂O) δ 7.80-7.74 (m, 1H), 6.11 (d, J=1.9 Hz, 1H), 5.91 (dd, J=8.0, 1.0 Hz, 1H), 4.53-4.43 (m, 1H), 4.41 (dt, J=6.3, 1.6 Hz, 1H), 4.02 (t, J=4.7 Hz, 2H), 3.56 (tt, J=7.6, 6.4 Hz, 1H), 1.46 (d, J=7.1 Hz, OH), 1.27 (dd, J=7.0, 1.0 Hz, 3H). ¹⁹F NMR (376 MHz, D₂O) δ −120.70 (dt, J=18.6, 4.5 Hz). ³¹P NMR (162 MHz, D₂O) δ 6.68. ESI-MS: m/z 412 ([M−H]⁻).

### Example 112. Uptake and Metabolism of EIDD-3232 in Tissue Culture Experiments Compared to EIDD-2749

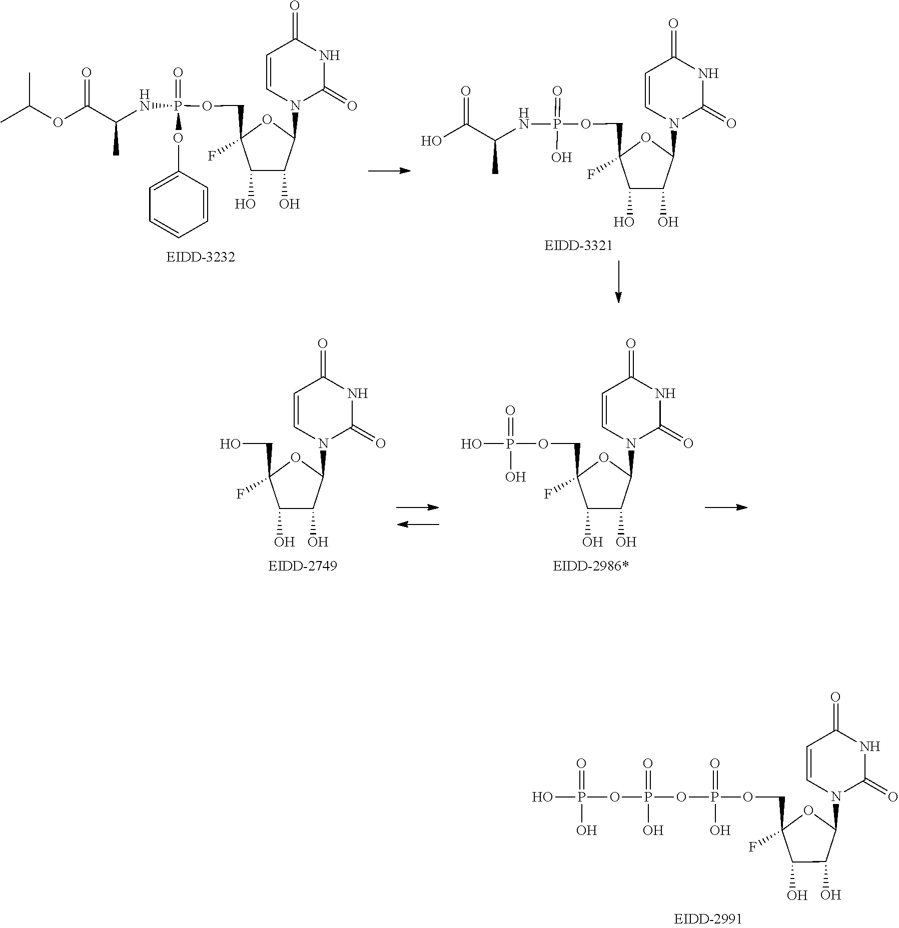 [SMILES: CC(C)OC(=O)[C@H](C)N[P@](=O)(OC[C@@]1(F)O[C@@H](n2ccc(=O)[nH]c2=O)[C@H](O)[C@@H]1O)Oc1ccccc1 ; C[C@H](NP(=O)(O)OC[C@@]1(F)O[C@@H](n2ccc(=O)[nH]c2=O)[C@H](O)[C@@H]1O)C(=O)O ; O=c1ccn([C@@H]2O[C@](F)(CO)[C@@H](O)[C@H]2O)c(=O)[nH]1 ; O=c1ccn([C@@H]2O[C@](F)(COP(=O)(O)O)[C@@H](O)[C@H]2O)c(=O)[nH]1 ; O=c1ccn([C@@H]2O[C@](F)(COP(=O)(O)OP(=O)(O)OP(=O)(O)O)[C@@H](O)[C@H]2O)c(=O)[nH]1]

The proposed metabolism of EIDD-3232 is given in the scheme above. Without wishing to be bound by a particular theory, it is believed that EIDD-3232 when administered orally can pass intact through the stomach and into the upper gut (see data in Examples 115-116), before being shunted to the liver via the hepatic portal system. Further, without wishing to be bound by a particular theory, it is believed EIDD-3232 is catabolized to its M1 metabolite, EIDD-3321, in the presence of esterases, and is then converted in cells to the EIDD-2986 monophosphate form by cellular phosphoramidases (such as HINT1), followed by phosphorylation by nucleotide kinases to a pharmaceutically active 5′-triphosphate form.

The uptake and metabolism of EIDD-3232 in cultured cells was assessed as described herein. Briefly, A549, HepG2, and Vero cells (ATCC) were plated at a seeding density of 0.35×10⁶ viable cells per well in 24-well plates and incubated overnight in complete Dulbecco's modified eagle medium (DMEM) at 37° C. with 5% CO₂ to allow the cells to attach to the wells. EIDD-3232 was diluted in complete DMEM at a concentration of 20 μM and EIDD-2749 was diluted in DMEM at 20 μM (A549) or 50 μM (Vero) and added to the cells at time=Oh. Cells were incubated at 37° C. with 5% CO₂ for 1, 2, 3, 4, 6, 16, and 24 hours before cells were extracted. At the appropriate timepoint cells were washed twice with Dulbecco's phosphate buffered saline and then extracted with a 70% acetonitrile solution which contained internal standards (IS). Cell extracts were transferred into microcentrifuge tubes and centrifuged at 20,000 rcf for 10 min at 4° C. Clarified extracts were then transferred to an HPLC vial for analysis by LC-MS/MS according to a qualified bioanalytical method.

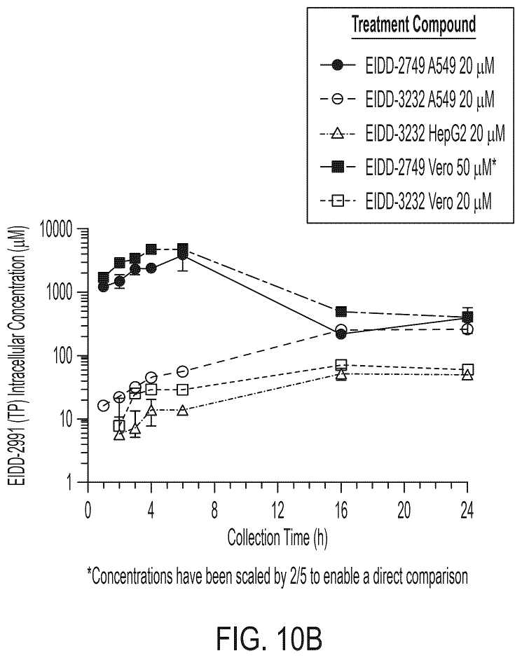

The data are shown in FIGS. 10A-10B. The data in FIG. 10A show that EIDD-3232 penetrates cells quickly and remains stable at a concentration of about 3-20 μM. EIDD-3321 was seen at low levels in A549 cells (10-20 μM) and HepG2 Cells (2-6 μM) (data not shown), whereas EIDD-2749 was not observed in any cell line at any time (data not shown). EIDD-3232 levels reach ˜60 μM in Vero cells, but the % CV was >100% at that time. The data in FIG. 10B show that cells incubated with EIDD-2749 produce higher intracellular levels of EIDD-2991 early in the incubation, but the difference in level decreases after 16-24 hours. EIDD-2991 is highest in A549 cells (260 μM) for cells incubated with EIDD-3232, followed by Vero cells (70 μM) and HepG2 cells (50 μM). The data are consistent with the following observations: (a) EIDD-3232 penetrates cells quickly and produces EIDD-2991 up to 260 μM (A549), 50 μM (HepG2), and 70 μM (Vero), whereas. EIDD-3321 and EIDD-2749 are not produced at significant levels (<20 μM); and (b) EIDD-2749 produces EIDD-2991 at much higher levels in A549 cells (˜15×) and in Vero cells (˜70×) but the differences are less drastic after 16-24 hours of incubation.

### Example 113. Pharmacokinetics of EIDD-3232, EIDD-3321, and EIDD-2749 in Mouse Plasma after Oral Dosing at 10 mg/kg of EIDD-3232 or 5 mg/kg of EIDD-2749

Briefly, female ICR (CD-1) mice, between seven to eight weeks old, were acclimated to their environment for at least two days prior to dosing. Mice were weighed at least once before dosing to determine the dosing volume. EIDD-3232 and EIDD-2749 were dissolved in a 10 mM trisodium citrate/0.5% Tween 80/Water vehicle. Mice were dosed by oral gavage with EIDD-3232 at a 10 mg/kg or with EIDD-2749 at 5 mg/kg. Blood samples were collected from three mice at each sample time point. Blood was collected at 0.25, 0.5, 1, 2, 4, 8, and 24 hours post-dose for EIDD-3232-dosed mice and 0.25, 0.5, 1, 2, 3, 4, 8, and 24 hours post dose for EIDD-2749-dosed mice. Whole blood was collected into a lithium-heparin or K2EDTA containing microtainer tubes and placed on ice until it could be processed (less than an hour). Whole blood samples were centrifuged at approximately 2000-times gravity for 10 minutes at 4° C. and the resulting plasma was transferred into a separate microcentrifuge tube and stored at −80° C. until it was processed for analysis by LC-MS. Immediately after blood collection mice were sacked under deep isoflurane anesthesia and mouse organs were harvested. Aliquots of mouse plasma were extracted with a solution of 70% acetonitrile in water that included internal standards and samples of mouse plasma from mice that were dosed with EIDD-2749 were extracted with acetonitrile that included an internal standard. Samples were clarified by centrifugation at 20,000×g at 4° C. for 10 min. The clarified supernatants were transferred to HPLC vials for analysis according to a qualified bioanalytical method.

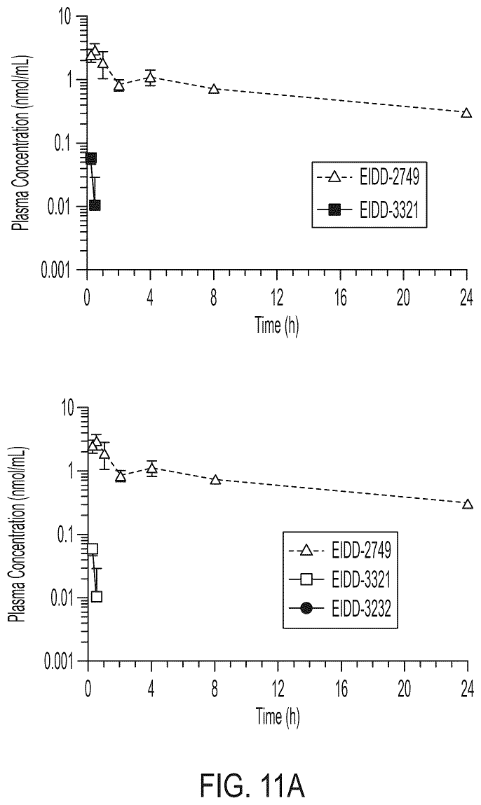

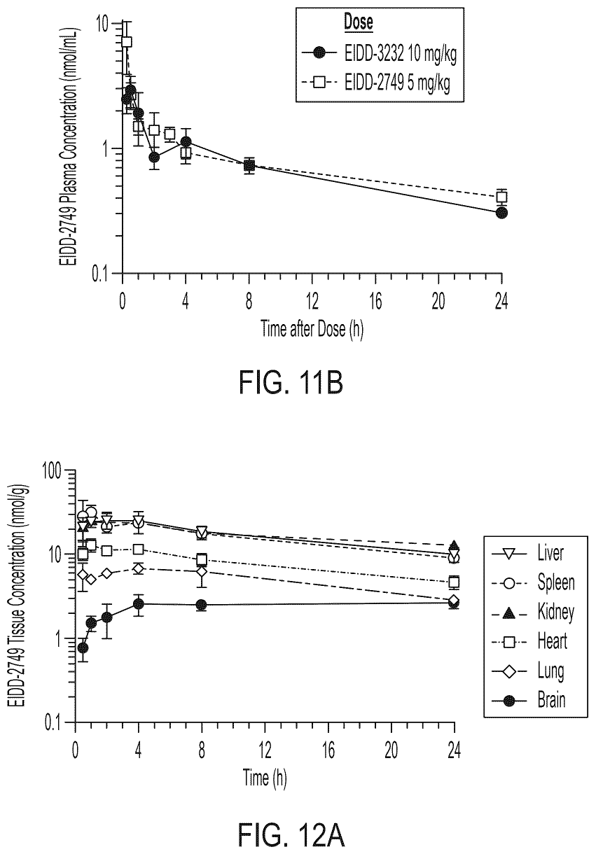

The data are shown in FIGS. 11A-11B, and various values calculated from these data are shown in the tables shown immediately below. The data in FIG. 11A show that EIDD-3232 was not observed at any timepoint under the conditions of this assay. In contrast, EIDD-3321 was present at low levels shortly after the dose, but was BQL 0.5 hours post-dose. Notably, EIDD-2749 is generated quickly, reaching maximum concentrations within 0.5 hours of the dose. The data in FIG. 11B show that oral dosing with EIDD-2749 provides higher plasma levels of EIDD-2749 0.25 hours post-dose, but levels are similar for all other timepoints for plasma levels of EIDD-2749 after oral dosing with either EIDD-2749 or EIDD-3232. In addition, it can be observed that exposures to EIDD-2749 were similar between the two dose groups, and that the observed terminal half-lives are similar between dose groups. The data are consistent with the observation that EIDD-3232 provides similar EIDD-2749 plasma PK levels compared to an equimolar dose of EIDD-2749.

### Calculated Parameters from Data in FIG. 11A

| | Tₘₐₓ | Cₘₐₓ | AUC_{inf} | t_{1/2} |
|---|---|---|---|---|
| Analyte | (h) | (nmol/mL) | (h*nmol/mL) | (h) |
| EIDD-3232 | N/A | BQL | N/A | N/A |
| EIDD-3321 | 0.25 | 0.0582 ± 0.0118 | 0.0174 | 0.101 |
| EIDD-2749 | 0.50 | 2.94 ± 0.826 | 22.6 | 11.1 |

### Calculated Parameters from Data in FIG. 11B

| | Tₘₐₓ | Cₘₐₓ | AUC_{inf} | t_{1/2} |
|---|---|---|---|---|
| Dose | (h) | (nmol/mL) | (h*nmol/mL) | (h) |
| EIDD-3232 | 0.50 | 2.94 ± 0.826 | 22.6 | 11.1 |
| EIDD-2749 | 0.25 | 7.1 ± 3.2 | 28.6 | 15.2 |

### Example 114. Comparison of Tissue Distribution after Oral Dosing of EIDD-3232 at 10 mg/kg or EIDD-2749 at 5 mg/kg

Briefly, female ICR (CD-1) mice, between seven to eight weeks old, were acclimated to their environment for at least two days prior to dosing. Mice were weighed at least once before dosing to determine the dosing volume. EIDD-3232 and EIDD-2749 were dissolved in a 10 mM trisodium citrate/0.5% Tween 80/Water vehicle. Mice were dosed by oral gavage with EIDD-3232 at a 10 mg/kg or with EIDD-2749 at 5 mg/kg (approximately equimolar oral doses of EIDD-3232 and EIDD-2749). At the appropriate timepoint, three mice were sacked under deep isoflurane anesthesia and brain, heart, kidney, liver, lung, and spleen tissues were collected and snap-frozen in liquid nitrogen. Tissues were collected at 0.5, 1, 2, 4, 8, and 24 hours post-dose for EIDD-3232-dosed mice and 0.25, 0.5, 1, 2, 3, 4, 8, and 24 hours post-dose for EIDD-2749-dosed mice. Frozen animal tissues were extracted with cold (4° C.) 70% acetonitrile in water by homogenization in a Lysera bead mill outfitted with a cryo cooling unit (Biotage, Uppsala, Sweden). The lysate was centrifuged at 20,000 rcf for 5 minutes at 4° C. and the clarified lysate was transferred to a LCMS vial and analyzed according to a qualified bioanalytical method.

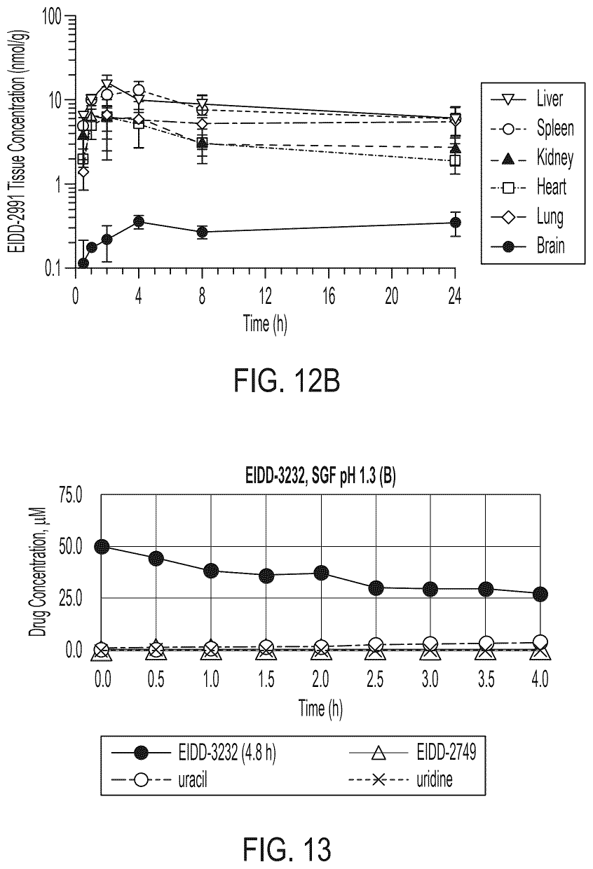

The data are shown in FIGS. 12A-12B for mice dosed with EIDD-3232. Various values calculated from these data are shown in the tables shown immediately below. EIDD-3232 was not detected in any tissues under the conditions of the described assay. In contrast, EIDD-3321 was detected at low levels (˜0.02 nmol/g) in the Kidney, but BQL in all other tissues (data not shown). Moreover, EIDD-2749 and EIDD-2991 were generated quickly and the highest levels of each were measured in the liver and spleen and the lowest levels were measure in the brain. The following observations are consistent with these data: (a) EIDD-3232 provides similar EIDD-2749 Cₘₐₓ and AUC₂₄ values in all tissues compared to an equimolar dose of EIDD-2749; and (b) EIDD-3232 increases the EIDD-2991 Cₘax and AUC₂₄ in all tissues (e.g., liver Cₘₐₓ increases about 15-fold, and AUC increases about 10-fold).

### Calculated Parameters from Data in FIGS. 12A-12B

| | | Dose | Tₘₐₓ | Cₘₐₓ | AUC₂₄ | AUC_{inf} | t_{1/2} |
|---|---|---|---|---|---|---|---|
| Analyte | Tissue | Compound | (h) | (nmol/g) | (h*nmol/g) | (h*nmol/g) | (h) |
| EIDD-2749 | Brain | EIDD-2749 | 24 | 4.40 | 71 | 188 | 17.7 |
| | | EIDD-3232 | 24 | 2.66 | 58.4 | 543 | 126 |
| | Heart | EIDD-2749 | 2 | 15.3 | 234.1 | 422 | 20.8 |
| | | EIDD-3232 | 1 | 12.9 | 190 | 302 | 16.6 |
| | Kidney | EIDD-2749 | 2 | 31.3 | 369.4 | 607 | 17.7 |
| | | EIDD-3232 | 1 | 26.3 | 425 | 1090 | 35.8 |
| | Liver | EIDD-2749 | 1 | 18.3 | 312.7 | 560 | 19.4 |
| | | EIDD-3232 | 2 | 25.2 | 409 | 637 | 15.8 |
| | Lung | EIDD-2749 | 2 | 5.9 | 103.1 | 210 | 24.0 |
| | | EIDD-3232 | 4 | 6.85 | 122 | 187 | 15.5 |
| | Spleen | EIDD-2749 | 2 | 31.6 | 406 | 766 | 22.5 |
| | | EIDD-3232 | 1 | 31.5 | 394 | 590 | 14.9 |
| EIDD-2991 | Brain | EIDD-2749 | 24 | 0.22 | 3.8 | 7.7 | 10.7 |
| | | EIDD-3232 | 4 | 0.356 | 7.06 | 12.0 | 9.81 |
| | Heart | EIDD-2749 | 3 | 3.00 | 39.7 | 68.3 | 18.0 |
| | | EIDD-3232 | 2 | 6.22 | 75.6 | 137 | 22.4 |
| | Kidney | EIDD-2749 | 8 | 0.69 | 12.5 | 22 | 17.4 |
| | | EIDD-3232 | 1 | 6.67 | 86.1 | 611 | 133 |
| | Liver | EIDD-2749 | 8 | 1.40 | 24.4 | 41.3 | 17.2 |
| | | EIDD-3232 | 2 | 16.3 | 202 | 457 | 29.0 |
| | Lung | EIDD-2749 | 4 | 2.60 | 46.4 | 119 | 30.1 |
| | | EIDD-3232 | 1 | 6.37 | 128 | 2400 | 286 |
| | Spleen | EIDD-2749 | 4 | 4.90 | 74 | 165 | 26.9 |
| | | EIDD-3232 | 4 | 13.0 | 190 | 593 | 46.7 |

### Example 115. Drug Metabolism: Stability of EIDD-3232 in Simulated Gastric Fluid (pH 1.3)

Briefly, EIDD-3232 was incubated in triplicate at 50.0 μM (1.25 μL of a 40 mM DMSO solution in 999 L) in simulated gastric fluid (SGF: 0.200 (w/v) NaCl in 0.700 (v/v) concentrated HCl, Ricca, Lot 2202943) in 2-mL HPLC vials. Vials were pre-incubated in LC autosampler set at 37° C. for at least 15 minutes prior to spiking the SGF with the drug. Vials were placed back in the 37° C.-autosampler and injections were made at times: 0, 0.5, 1, 1.5, 2, 2.5, 3, 3.5, and 4 hours and analyzed by LC-MS/MS. No internal standard was used. Calibration curves for EIDD-2749 (nuc), uracil, and uridine were prepared in SGF. Half-lives (t_{1/2}) were calculated by plotting the natural logarithm of the analyte concentration vs. time and obtaining the slope of the line.

LC-MS/MS was carried out using an Agilent Eclipse XDB-C8 column (150×4.6 mm, 3.5 m) for EIDD-3232 and a SeQuant ZIC-pHILIC polymeric column (100×4.6 mm, 5 m) for EIDD-2749, uracil and uridine. For EIDD-3232, the mobile phase was isocratic using 18% A: 100 mM ammonium formate in water+0.1% (v/v) formic acid and 82% B: acetonitrile+0.1% (v/v) formic acid at a flow rate of 1000 L/min with a run time of about 3 minutes. For EIDD-2749, uracil and uridine, the mobile phase was isocratic using 27% A: 50 mM ammonium bicarbonate in water, pH 9.8 and 73% B: acetonitrile at a flow rate of 750 L/min with a run time of about four minutes.

The data are shown in FIG. 13. The calculated half-life of EIDD-3232 in SGF was 4.8 hours (calculated using the data in FIG. 13), and the EIDD-2749 production was negligible. The only degradant observed was uracil, which was <8% of the time-zero concentration after 4 hours of incubation.

### Example 116. Drug Metabolism: Stability of EIDD-3232 in Simulated Intestinal Fluid (pH 5.2)

Briefly, EIDD-3232 was incubated in triplicate at 50.0 μM (1.25 μL of a 40 mM DMSO solution in 999 μL) in simulated intestinal fluid (SIF: 0.2% (w/v) NaCl in 0.7% (v/v) concentrated HCl, Ricca, Lot 2202943) in 2-mL HPLC vials. Vials were pre-incubated in LC autosampler set at 37° C. for at least 15 minutes prior to spiking the SGF with the drug. Vials were placed back in the 37° C.-autosampler and injections were made at times: 0, 0.5, 1, 1.5, 2, 2.5, 3, 3.5, and 4 hours and analyzed by LC-MS/MS. No internal standard was used. Calibration curves for EIDD-2749 (nuc), uracil, and uridine were prepared in SIF, pH 5.2. Half-lives (t_{1/2}) were calculated by plotting the natural logarithm of the analyte concentration vs. time and obtaining the slope of the line.

LC-MS/MS was carried out using an Agilent Eclipse XDB-C₈ column (150×4.6 mm, 3.5 μm) for EIDD-3232 and a SeQuant ZIC-pHILIC polymeric column (100×4.6 mm, 5 m) for EIDD-2749, uracil and uridine. For EIDD-3232, the mobile phase was isocratic using 18% A: 100 mM ammonium formate in water+0.1% (v/v) formic acid and 82% B: acetonitrile+0.1% (v/v) formic acid at a flow rate of 1000 L/min with a run time of about 3 minutes. For EIDD-2749, uracil and uridine, the mobile phase was isocratic using 27% A: 50 mM ammonium bicarbonate in water, pH 9.8 and 73% B: acetonitrile at a flow rate of 750 L/min with a run time of about four minutes.

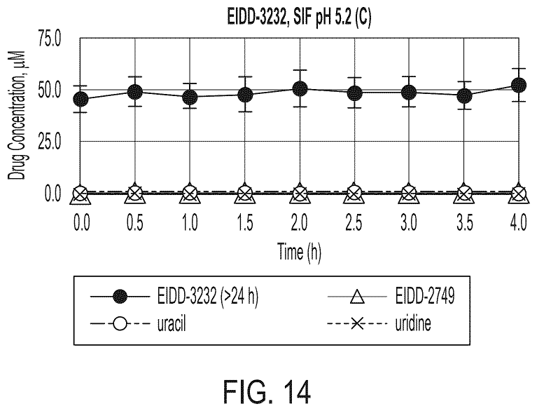

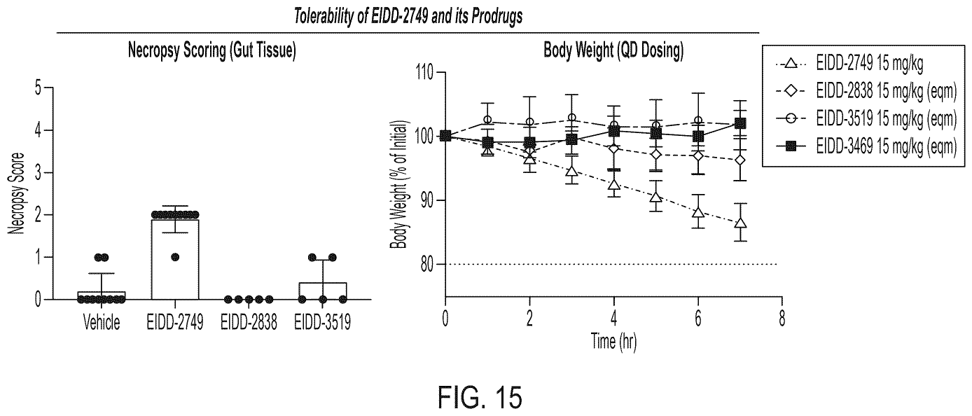

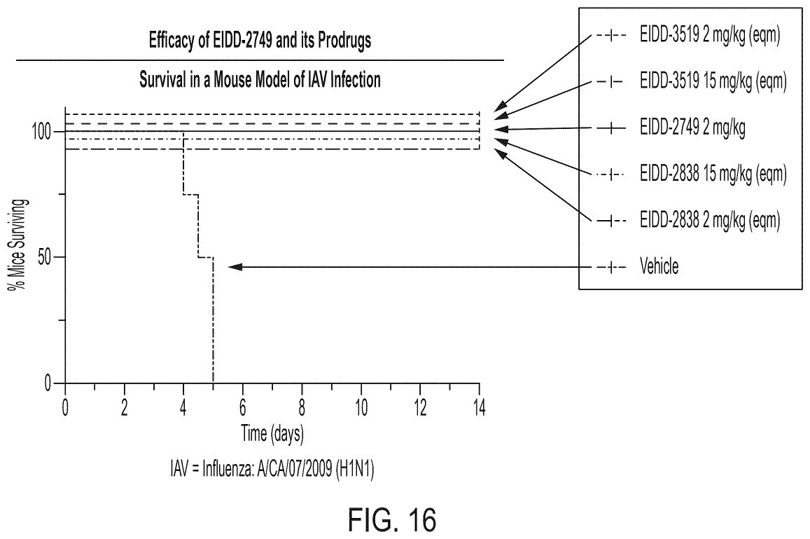

The data are shown in FIG. 14. The calculated half-life of EIDD-3232 in SIF pH 5.2 was >24 hours (calculated using the data in FIG. 14), and the EIDD-2749 production was negligible. None of the metabolites/degradants was detected above 0.125 μM (lower limits of quantitation), except for uracil at 3 hours, average concentration was 0.152 μM.

### Example 117. Anti-Viral Activity of EIDD-3232

Anti-viral activity of EIDD-3232 was determined as discussed herein above (e.g., see Example 48 and elsewhere herein above), for anti-viral activity against CHIKV, Coxsackie virus B3, DENV-2, EEEV, ENTV68, ENTV71, Flu A H₁N1, HRV-14, JUNV, MERS-CoV, MEV, PIV-3, POV-1, POWV(Lineage 1), RSV, RVFV, SARS-CoV-2, TCRV, VEEV, WNV, and YFV. The data are shown immediately below.

| | | Cell | EC₅₀ | EC₉₀ | CC₅₀ |
|---|---|---|---|---|---|
| Virus | Strain | Line | (μM) | (μM) | (μM) |
| CHIKV | S27, ATCC | Huh7 | 5.7 | 4.5 | >32 |
| | VR-64 | | | | |
| Coxsackie | HA201933 | Huh7 | 18 | | >32 |
| virus B3 | | | | | |
| DENV-2 | New Guinea C | Huh7 | 4.2 | 19 | >32 |
| EEEV | FL39-939 | Vero 76 | >32 | | >32 |
| ENTV68 | US/KY/14- | RD | 18 | | >32 |
| | 18953 | | | | |
| ENTV71 | Tainan/4643/98 | Vero 76 | >32 | | >32 |
| Flu A H1N1 | California/07/ | MDCK | 1.3 | 1.5 | >32 |
| | 2009 | | | | |
| HRV-14 | 1059.0 | Hela | >32 | | >32 |
| | | Ohio | | | |
| JUNV | Candid #1 | Vero | 2 | 2.2 | >32 |
| MERS-CoV | EMC | Vero 76 | >32 | | >32 |
| MEV | CC | Vero 76 | 9.6 | | >32 |
| PIV-3 | 14702.0 | MA-104 | >32 | | >32 |
| POV-1 | Mahoney | Vero 76 | >32 | | >32 |
| POWV(Lineage 1) | LB | BHK-21 | >32 | | >32 |
| RSV | A2 | MA-104 | >32 | | >32 |
| RVFV | MP-12 | Huh7 | 6.4 | 7.4 | >32 |
| SARS-CoV-2 | USA_WA1/2020 | Vero E6 | >17 | | 17 |
| TCRV | TRVL 11573 | Vero | 2.8 | 3.9 | >32 |
| VEEV | TC-83 | Vero 76 | >32 | | >32 |
| WNV | Kern 515, | Vero 76 | >32 | | >32 |
| | WN02 | | | | |
| YFV | YFV 17D | Huh7 | 15 | | >32 |

### Example 118. Anti-viral Activity of EIDD-3382, -3383, and 3445

To investigate the impact of the phosphoramidate moiety on antiviral activity and PK profile, three analogs featuring a R, R configuration of the phosphoramidate side chain (EIDD-3382), a less hindered glycine side chain (EIDD-3383), and a more hindered valine side chain (EIDD-3445) were prepared. The herein described synthesis methods were used to prepare EIDD-3382, -3383, and 3445, having the structures given by the following formulas:

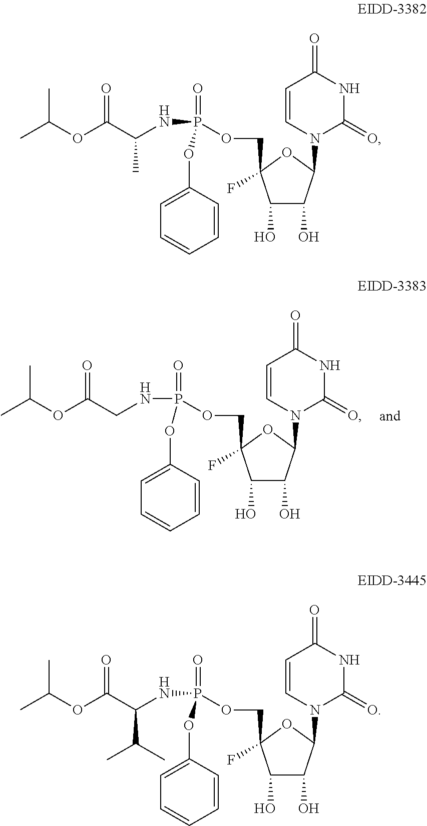 [SMILES: CC(C)OC(=O)[C@@H](C)N[P@@](=O)(OC[C@@]1(F)O[C@@H](n2ccc(=O)[nH]c2=O)[C@H](O)[C@@H]1O)Oc1ccccc1 ; CC(C)OC(=O)CNP(=O)(OC[C@@]1(F)O[C@@H](n2ccc(=O)[nH]c2=O)[C@H](O)[C@@H]1O)Oc1ccccc1 ; CC(C)OC(=O)[C@@H](N[P@](=O)(OC[C@@]1(F)O[C@@H](n2ccc(=O)[nH]c2=O)[C@H](O)[C@@H]1O)Oc1ccccc1)C(C)C]

In vitro cell based data showed that EIDD-3445 has some activity against YFV in Huh7 cell line (EC₅₀=18 μM), similar to EIDD-3232 (EC₅₀=15 μM). However, EIDD-3382 and EIDD-3383 were found to be inactive (EC₅₀>32 μM). In addition, EIDD-3382, EIDD-3383, and EIDD-3445 are not active against DENV-2 in Huh7 cell line (EC₅₀>32 μM), however, EIDD-3232 is active (EC₅₀=4.2 μM). This indicates that the amino acid moiety and the stereochemical relationship of the phosphorus atom play an important role in observed in vitro antiviral activity. A tissue distribution study of EIDD-3232 conducted in mice demonstrated high concentrations of 5′-triphosphate in the lung, kidney, liver, and spleen. This data led us to investigate various analogs to optimize their PK profile. The current and most efficient route to analogs of EIDD-3232 begins with intermediate 1, 2′,3′-Cbz protected EIDD-2749, which was coupled with the corresponding phosphoramidate reagent. Global deprotection by hydrogenolysis gave the corresponding prodrug 2. Using this synthetic route, phosphoramidate analogs EIDD-3382, EIDD-3383 and EIDD-3445 were also prepared and their PK profile will be evaluated.

### Example 119. General Procedure for Synthesis of Disclosed Alpha Alkoxyester Analogs of Disclosed Compounds

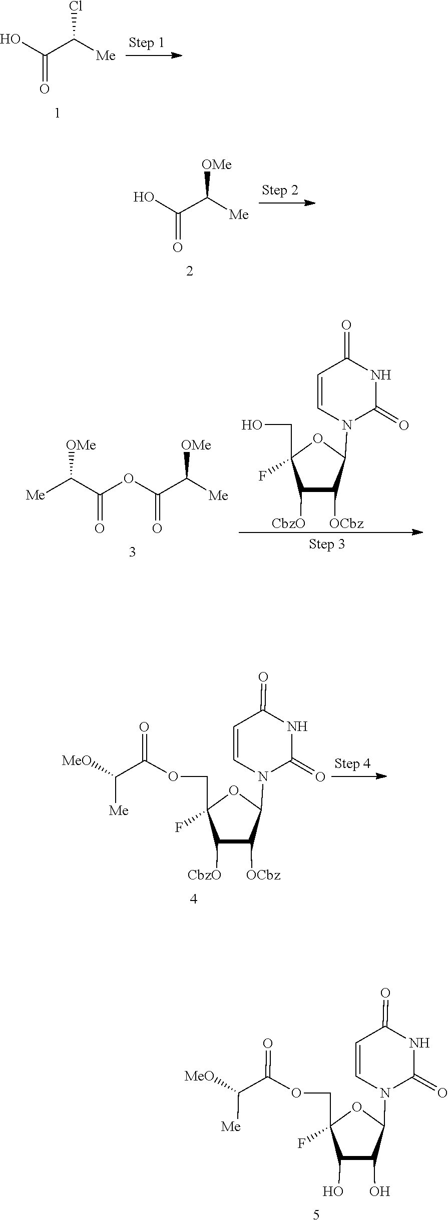

Step 1: (S)-2-Chloropropanoic acid 1 (1 eq) was added to a flask with 25 wt % of NaOMe in MeOH (3 eq) under argon. The solution was heated to 60° C. until the conversion is complete. After cooling to rt, the pH was adjusted with 4N HCl in dioxane to 7. The mixture was filtered to remove salts and washed with MeOH (×3). The filtrate was concentrated in vacuo, redissolved in water and acidified to pH=2 with 6N HCl, then extracted with EtOAc. Organic layers were dried (Na₂SO₄), filtered and concentrated in vacuo to afford 2, which is used directly for the next step.

Step 2: To a flask charged with intermediate 2 was added dry DCM under argon, then MsCl (0.5 eq) was added. The flask was cooled to 0° C. and Et₃N (1 eq) was added dropwise. After addition, the mixture was stirring at rt for 1 h, then water was added to quench the reaction. Organic layer was washed with water (×2), concentrated in vacuo to afford intermediate 3, which was used directly for the next step.

Step 3: To a solution of 2′,3′-Cbz protected 4′-F-uridine (1 eq) was added pyridine, followed by the addition of anhydride intermediate 3 (2 eq) and DMAP (0.1 eq) at 0° C. After addition, the mixture was allowed to stir at rt until the reaction is complete. Then solvent was removed in vacuo and the residue was treated dissolved in EtOAc and washed with 1N HCl, water, dried (Na₂SO₄) and purified by SiO₂ column chromatography to afford intermediate 4.

Step 4: Intermediate 4 (1 eq) was hydrogenated with a H₂ balloon with 10% Pd/C (0.1 eq) in MeOH. After 1 h, Pd catalyst was filtered, and the filtrate was concentrated in vacuo. The material was purified by SiO₂ column chromatography to afford compound 5.

### Example 120. Synthesis of EIDD-3577

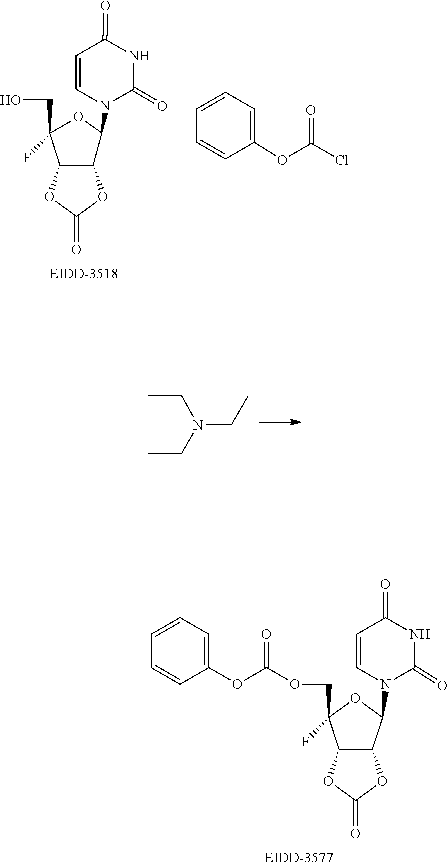

To a 25 mL pear-shaped flask charged with 1-[(3aS,4S,6R,6aR)-4-fluoro-4-(hydroxymethyl)-2-oxo-6,6a-dihydro-3aH-furo[3,4-d][1,3]dioxol-6-yl]pyrimidine-2,4-dione (0.15 g, 0.52 mmol) (EIDD-3518) was added dry THF (5.2 mL) to give a cloudy solution. This was vacuumed and charged with argon. Then the flask was cooled to 0° C. and triethylamine (0.15 mL, 1.04 mmol) was added, followed by phenyl chloroformate (0.08 mL, 0.62 mmol) to give a white suspension. After 5 min, ice-water bath was removed. After 2 h, TLC showed no SM, then water and EtOAc was added, but the mixture did not become clear. Then solvent was removed in vacuo with Celite, and the crude material was purified by ISCO column chromatography (12 g) eluting with a mixture of EtOAc and hexanes to afford EIDD-3577 (140 mg, 66%) as a white solid. ¹H NMR (400 MHz, CD₃CN) 6 9.34 (s, 1H), 7.53-7.42 (m, 3H), 7.40-7.30 (m, 1H), 7.29-7.21 (m, 2H), 6.05 (t, J=0.9 Hz, 1H), 5.81-5.60 (m, 3H), 4.60 (s, 1H), 4.56 (d, J=1.9 Hz, 1H). ¹³C NMR (101 MHz, CD₃CN) δ 162.77, 153.50, 153.03, 151.10, 150.35, 144.06, 129.69, 126.46, 121.13, 115.29 (d, J=235.3 Hz), 102.42, 95.20, 81.45, 78.83 (d, J=19.7 Hz), 66.39 (d, J=33.4 Hz). ¹⁹F NMR (376 MHz, CD₃CN) δ −114.18 (td, J=14.4, 10.7 Hz).

### Example 121. Synthesis of EIDD-3590

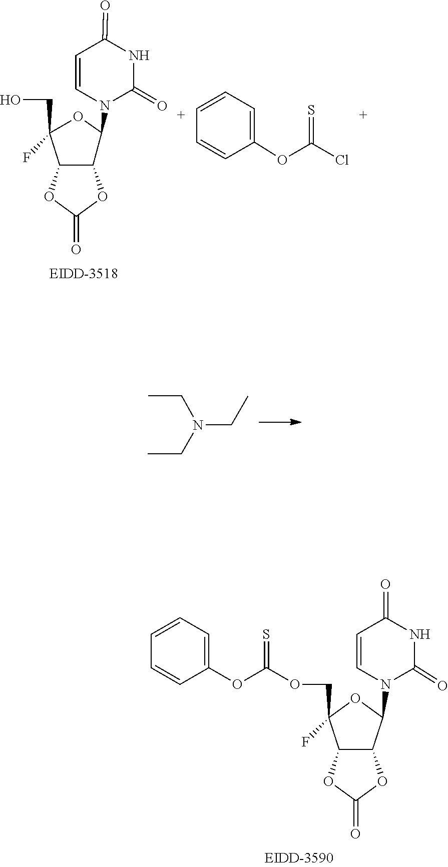

To a 50 mL pear-shaped flask charged with 1-[(3aS,4S,6R,6aR)-4-fluoro-4-(hydroxymethyl)-2-oxo-6,6a-dihydro-3aH-furo[3,4-d][1,3]dioxol-6-yl]pyrimidine-2,4-dione (0.30 g, 1.04 mmol) (EIDD-3518) was added dry THF (10.41 mL) to give a clear solution. This was vacuumed and charged with argon. Then Triethylamine (0.29 mL, 2.08 mmol) was added. After cooling to 0° C., O-Phenylchlorothionoformate (0.17 mL, 1.25 mmol) was added to give a yellow suspension. After 10 min, ice-water bath was removed, and the mixture was stirring at rt. After 5 h, TLC showed no SM, then solvent was removed in vacuo. The crude residue was dissolved in DCM and purified by ISCO column chromatography (12 g) eluting with a mixture of EtOAc and hexanes to afford EIDD-3590 (220 mg, 50%) as a white solid. 1H NMR (400 MHz, CD3OD) δ 7.72 (d, J=8.1 Hz, 1H), 7.55-7.42 (m, 2H), 7.37-7.29 (m, 1H), 7.22-7.09 (m, 2H), 6.22 (s, 1H), 5.80 (dd, J=10.9, 7.3 Hz, 1H), 5.77-5.72 (m, 2H), 4.89 (s, 2H). 19F NMR (376 MHz, CD3OD) δ −114.02 (dt, J=18.1, 11.3 Hz). ¹³C NMR (101 MHz, CD₃CN) δ 194.76, 162.77, 153.60, 153.47, 150.40, 144.06, 129.84, 126.95, 121.77, 115.18 (d, J=236.2 Hz), 102.45, 95.29, 81.52, 79.02 (d, J=19.5 Hz), 71.39 (d, J=31.4 Hz).

### Example 122. Synthesis of EIDD-3595

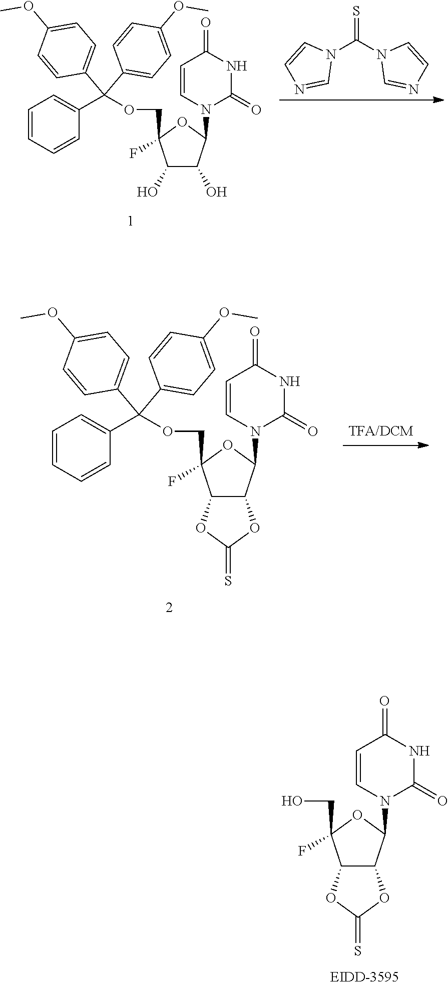 [SMILES: COc1ccc(C(OC[C@@]2(F)O[C@@H](n3ccc(=O)[nH]c3=O)[C@H](O)[C@@H]2O)(c2ccccc2)c2ccc(OC)cc2)cc1 ; S=C(n1ccnc1)n1ccnc1 ; COc1ccc(C(OC[C@@]2(F)O[C@@H](n3ccc(=O)[nH]c3=O)[C@@H]3OC(=S)O[C@@H]32)(c2ccccc2)c2ccc(OC)cc2)cc1 ; O=c1ccn([C@@H]2O[C@](F)(CO)[C@H]3OC(=S)O[C@@H]23)c(=O)[nH]1]

To a 25 mL round bottom flask was added a dry DCM (3.4 mL) solution of 1-[(2R,3R,4S,5S)-5-[[bis(4-methoxyphenyl)-phenyl-methoxy]methyl]-5-fluoro-3,4-dihydroxy-tetrahydrofuran-2-yl]pyrimidine-2,4-dione (0.38 g, 0.67 mmol). This was vacuumed and charged with argon. Then 1,1′-THIOCARBONYLDIIMIDAZOLE (179.93 mg, 1.01 mmol) was added all at once to give immediately a light brown (dark orange solution). This was vacuumed and charged with argon. The mixture was stirring overnight at rt. After stirring for 22.5 h, the mixture was concentrated in vacuo (bath temp 40° C.). The crude residue was diluted with DCM and purified by ISCO column chromatography (12 g) eluting with a mixture of EtOAc and hexanes to afford 2 (0.28 g, 69%).

To a 50 mL pear-shaped flask charged with 1-[(3aS,4S,6R,6aR)-4-[[bis(4-methoxyphenyl)-phenyl-methoxy]methyl]-4-fluoro-2-thioxo-6,6a-dihydro-3aH-furo[3,4-d][1,3]dioxol-6-yl]pyrimidine-2,4-dione (0.30 g, 0.49 mmol) (white glassy solid, stored in the fridge over the weekend) was added dry DCM (9.8909 mL) to give a colorless solution. This was vacuumed and charged with argon. The flask was cooled to 0° C. and then TFA (0.08 mL, 0.99 mmo) was added dropwise to give an orange solution. The mixture was stirring at 0° C. After 2.5 h, TLC showed a little SM, then ice-water bath was removed, the suspension was stirring at rt. After 1.5 h, the mixture was cooled to 0° C. and Et3N (0.35 mL) was added dropwise to give a light-yellow solution. After removing ice-water bath and stirred at rt for 5 min, it was concentrated in vacuo (bath temp 40° C.). The residue was diluted with DCM and purified by ISCO column chromatography (12 g) eluting with a mixture of EtOAc and hexanes to afford EIDD-3595 (120 mg, 80%). 1H NMR (400 MHz, CD3CN) δ 9.11 (s, 1H), 7.43 (d, J=8.2 Hz, 1H), 6.07 (d, J=1.0 Hz, 1H), 5.96-5.77 (m, 2H), 5.68 (d, J=8.1 Hz, 1H), 3.93-3.68 (m, 2H), 3.50 (s, 1H). ¹³C NMR (101 MHz, CD₃CN) δ 191.21, 162.79, 150.23, 143.94, 117.52 (d, J=237.4 Hz, 102.36, 94.43, 87.06, 83.07 (d, J=20.3 Hz), 61.71 (d, J=33.4 Hz). 19F NMR (376 MHz, CD3CN) δ −115.98 (dt, J=17.6, 11.1 Hz).

### Example 123. Synthesis of EIDD-3539

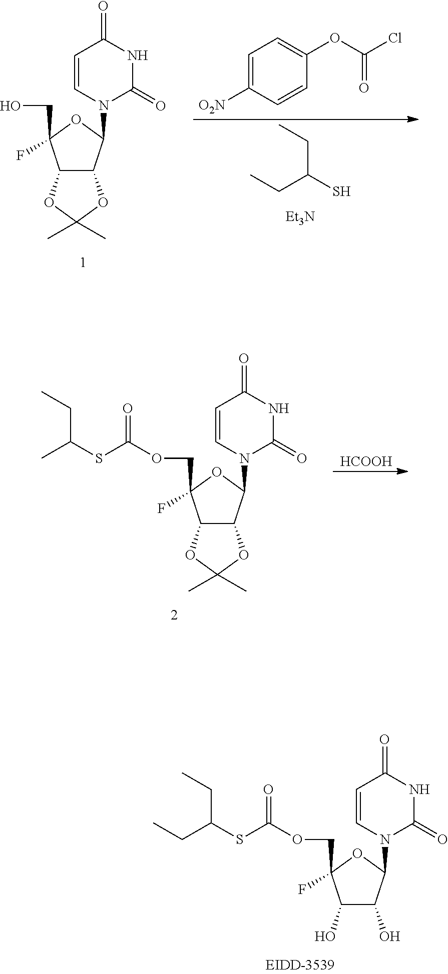

Into a solution of compound 1 (197 mg, 0.65 mmol) in THF (5 mL) was added triethylamine (272 μL, 2.0 mmol) and 4-nitrophenyl chloroformate (157 mg, 0.78 mmol), respectively, at 0° C. (slight exothermic). After stirring for 1.0 h at r.t., pentane-3-thiol (102 mg, 0.98 mmol) in THF (1 mL) and DMAP (32 mg, 0.26 mmol) were added. After stirring for 3 days at 60° C., the reaction mixture was concentrated in vacuo, and then purified on a SiO2 (12 g) column chromatography (Hex/EtOAc=8/2 to 6/4) to give slight impure TM, which was re-purified on a SiO2 (12 g) column chromatography (DCM/MeOH=100/0 to 98/2) to afford 240 mg of 2 as a white foam (85%).

A mixture of 2 (240 mg, 0.56 mmol) and 80% formic acid (5 mL) was stirred for 4 h at r.t. After concentration, the crude was re-dissolved with MeOH and concentrated, repeatedly (×2), and then purified on a SiO2 (12 g) column chromatography (DCM/MeOH=100/0 to 96/4), followed by lyophilization, to afford EIDD-3539 (125 mg, 57%). 1H NMR (400 MHz, CD3OD) δ 7.56 (d, J=8.1 Hz, 1H), 5.97 (d, J=2.1 Hz, 1H), 5.70 (d, J=8.1 Hz, 1H), 4.56 (dd, J=11.8, 7.1 Hz, 1H), 4.45 (dd, J=18.6, 6.7 Hz, 1H), 4.44-4.33 (m, 2H), 3.24 (tt, J=7.8, 5.5 Hz, 1H), 1.81-1.52 (m, 4H), 0.98 (td, J=7.4, 3.0 Hz, 6H).

### Example 124. Synthesis of EIDD-3521

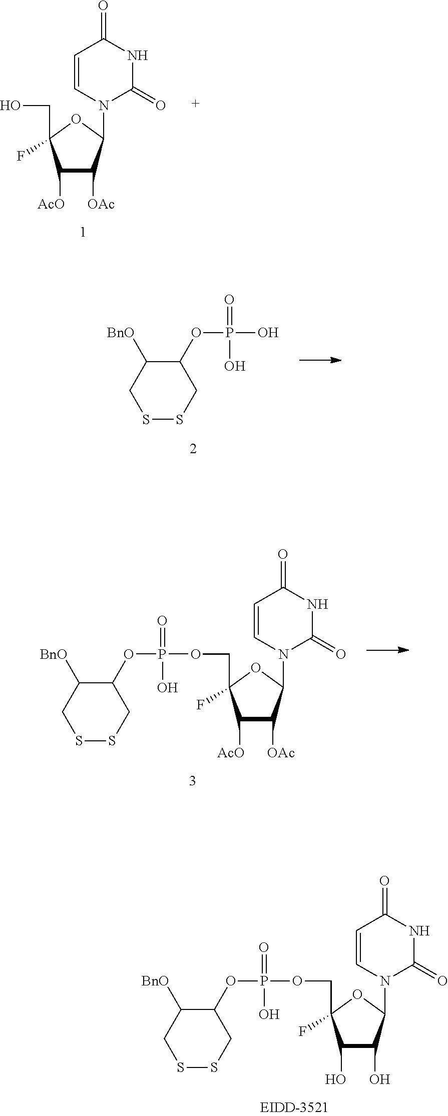

In a 50 mL rb was added intermediate 1 [(2R,3R,4S,5S)-4-acetoxy-2-(2,4-dioxopyrimidin-1-yl)-5-fluoro-5-(hydroxymethyl)tetrahydrofuran-3-yl]acetate (400.00 mg, 1.16 mmol), [rac-(4R,5R)-5-benzyloxydithian-4-yl]dihydrogen phosphate 2 (484.06 mg, 1.5 mmol) and anhydrous pyridine (10 mL). The mixture was then concentrated and co-evaporated with anhydrous pyridine (3×10 mL). The resulting residue was dissolved in pyridine (8 mL) and treated with N,N′-DICYCLOHEXYLCARBODIIMIDE (357.52 mg, 1.73 mmol). The mixture formed a white precipitate as it was heated to 65° C. with oil bath. The mixture turned slightly tan still with precipitate. The mixture was treated with 4-DIMETHYLAMINOPYRIDINE (141.13 mg, 1.16 mmol) and N,N′-DIISOPROPYLCARBODIIMIDE (0.27 mL, 1.73 mmol) and stirred at 45° C. for 36 h. TLC (20% methanol in DCM in NH₄OH) indicated mostly an new slower moving component. The mixture was concentrated to dryness. The mixture was dissolved in methanol and treated with Celite (3 g). After concentrating the solid was dry loaded and purified by column chromatography over silica gel using a 40 g silica column that was neutralized with 20% methanol in DCM with 2% ammonium hydroxide. After equilibrating the column with 20% methanol in DCM. The column was conditioned with DCM and then eluted with gradient to afford intermediate 3.

Intermediate 3 [rac-(2R,3R,4S,5S)-4-acetoxy-5-[[(5-benzyloxydithian-4-yl)oxy-hydroxy-phosphoryl]oxymethyl]-2-(2,4-dioxopyrimidin-1-yl)-5-fluoro-tetrahydrofuran-3-yl]acetate (460.00 mg, 0.71 mmol) was dissolved in methanol (7 mL) and treated dropwise with AMMONIUM HYDROXIDE (0.55 mL, 4.24 mmol). Mixture was concentrated. Co-evaporated with methanol. The crude material was purified by column chromatography over silica gel using the Comb Flash equipped with a 24 g silica cartridge. The cartridge was first condition with 20% methanol in DCM w/2% ammonium hydroxide followed by 10% methanol in DCM. The crude was then eluted with a DCM/methanol gradient to give a white solid that 1H, 19F, 31P NMR analysis were consistent with product structure. This material was dissolved in 8 mL of DI water, frozen, and lyophilized. Isolated (5-benzyloxydithian-4-yl) [rac-(2S,3S,4R,5R)-5-(2,4-dioxopyrimidin-1-yl)-2-fluoro-3,4-dihydroxy-tetrahydrofuran-2-yl]methyl hydrogen phosphate EIDD-3521 (280 mg, 67.597% yield) as a white solid.

¹H NMR (400 MHz, CD3OD, diastereomeric mixture) δ 7.73 (d, J=8.1 Hz, 1H, diastereomer1), 7.68 (d, J=8.1 Hz, 1H, diastereomer 2), 7.39 (td, J=8.2, 1.4 Hz, 2H), 7.36-7.27 (m, 2H), 7.27-7.19 (m, 1H), 6.15 (d, J=2.9 Hz, 1H, diastereomer 2), 6.13 (d, J=2.8 Hz, 1H, diastereomer 1), 5.78 (d, J=6.6 Hz, 1H, diastereomer 2), 5.76 (d, J=6.6 Hz, 1H, diastereomer 1), 4.79-4.61 (m, 2H), 4.41 (ddd, J=15.8, 14.3, 6.4 Hz, 1H), 4.29-3.99 (m, 4H), 3.63-3.46 (m, 2H), 3.27-3.17 (m, 1H), 3.03-2.92 (m, 1H), 2.88 (ddd, J=13.8, 9.3, 4.8 Hz, 1H). ¹³C NMR (101 MHz, CD3OD) δ 165.95, 165.89, 151.97, 142.32, 139.73, 139.69, 129.35, 129.32, 129.18, 129.03, 128.68, 128.63, 117.41 (d, J=236.9 Hz), 118.59, 116.23, 103.66, 103.53, 93.30, 93.10, 73.72, 73.61, 73.44, 70.62, 70.54, 70.42, 70.34, 64.51, 64.03. ³¹P NMR (162 MHz, CD3OD) δ −1.68. ¹⁹F NMR (376 MHz, CD3OD) δ −123.15 (d, J=16.1 Hz, diastereomer 1),−123.48 (d, J=15.8 Hz, diastereomer 2).

### Example 125. Synthesis of EIDD-3519

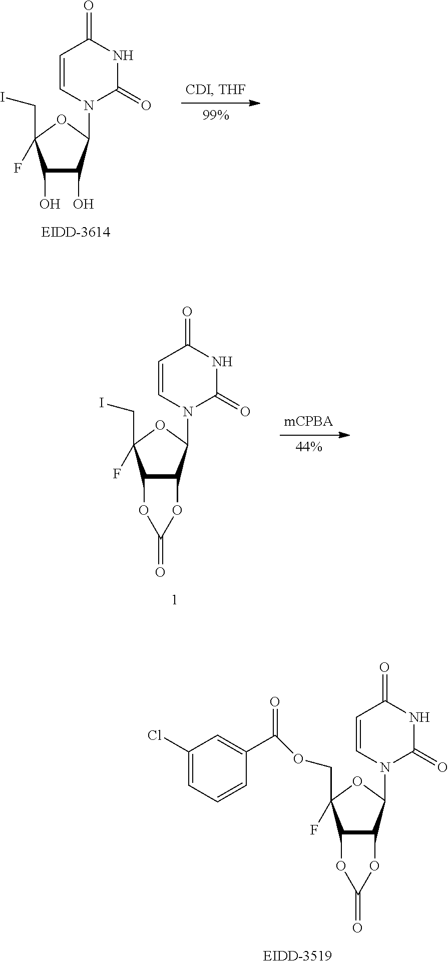

To a stirred suspension of EIDD-3614 (25 g, 67.188 mmol) in THF (336 mL) was charged carbonyl di imidazole (16.211 g, 99.972 mmol). Reaction was stirred rt 1 h was then concentrated to a paste. Residue was purified by silica gel chromatography ethyl acetate in hexanes 50-66% to provide intermediate 1 (26.7 g 67.072 mmol) as a white foam.

A buffer of tetrabutylammonium hydrogen sulfate (9.85 g, 29.014 mmol) and potassium phosphate dibasic (6.89 g, 39.565 mmol) in water (110 mL) was prepared, and then charged to a solution of intermediate 1 (10.5 g, 26.377 mmol) in dichloromethane (266 mL). Reaction was charged with 3-chloroperbenzoic acid (30.35 g, 131.8 8 mmol) and was vigorously stirred overnight. After 18 h reaction was charged with 80 mL sat aq sodium bicarbonate and 20 mL sat aq sodium thiosulfate. Quench was stirred 1 h and then the layers were separated. Aqueous was extracted 2×200 mL dichloromethane. The combined organics were washed 2×100 mL sat aq sodium bicarbonate and brine (100 mL). The dried (sodium sulfate) extracts were concentrated in vacuo and purified via silica gel chromatography 25-100% ethyl acetate in hexanes. Concentrated fractions were triturated with methyl tert buyl ether to provide EIDD-3519 (4.9 g, 44% yield) (three crops combined).

¹H NMR (400 MHz, CD₃CN) δ 9.17 (s, 1H), 8.06 (ddd, J=2.2, 1.6, 0.5 Hz, 1H), 8.02 (ddd, J=7.8, 1.6, 1.1 Hz, 1H), 7.71 (ddd, J=8.1, 2.2, 1.1 Hz, 1H), 7.60-7.52 (m, 1H), 7.44 (d, J=8.1 Hz, 1H), 6.05 (s, 1H), 5.78 (ddd, J=11.7, 7.3, 0.5 Hz, 1H), 5.72-5.64 (m, 2H), 4.74-4.52 (m, 2H).

### Example 126. General Procedure for the Synthesis of Disclosed 2′,3′-Cyclic Thiocarbonates

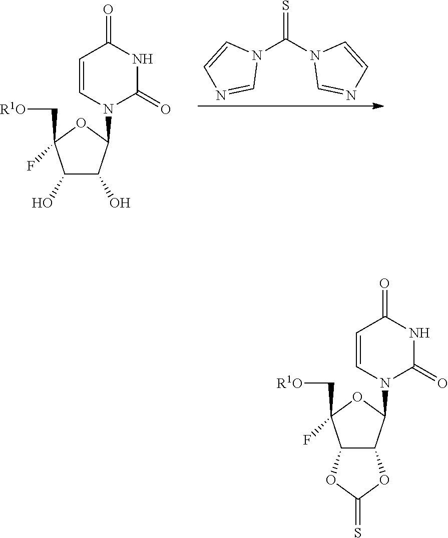

A 5′-substituted nucleoside (1 eq) was dissolved in either DCM or THF, then thio carbonyl di imidazole (1.25 eq) was added. After overnight reaction, solvent was removed in vacuo and purified by ISCO column chromatography to afford the product.

### Example 127. Synthesis of EIDD-3563

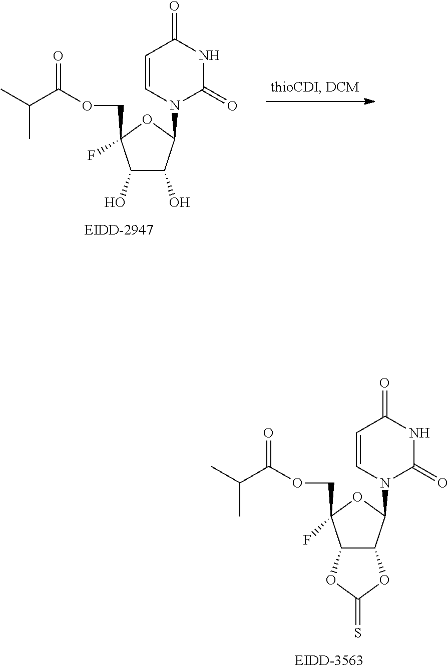 [SMILES: CC(C)C(=O)OC[C@@]1(F)O[C@@H](n2ccc(=O)[nH]c2=O)[C@H](O)[C@@H]1O ; CC(C)C(=O)OC[C@@]1(F)O[C@@H](n2ccc(=O)[nH]c2=O)[C@@H]2OC(=S)O[C@@H]21]

To a stirred suspension of EIDD-2947 (500 mg, 1.50 mmol) in dichloromethane (7.5 mL) was charged thio carbonyl di imidazole (335 mg, 1.88 mmol). Reaction was stirred rt 18 h was then concentrated to a paste. Residue was purified by silica gel chromatography methanol in dichloromethane 0-5% to provide EIDD-3563 (490 mg, 87% yield) as a white foam.

¹H NMR (400 MHz, CD₃CN) δ 9.27 (s, 1H), 7.46-7.39 (m, 1H), 6.09 (d, J=1.1 Hz, 1H), 5.92-5.81 (m, 2H), 5.72-5.65 (m, 1H), 4.49-4.38 (m, 2H), 2.66 (hept, J=7.0 Hz, 1H), 1.19 (d, J=7.0 Hz, 6H).

### Example 128. Synthesis of EIDD-3372

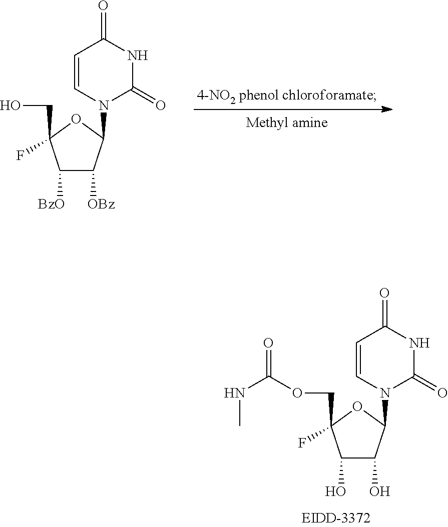

To a 0° C. solution of bis benzoyl 4′ fluoro uridine (941 mg, 2.0 mmol) in THF (10 mL) was added triethyl amine (840 μL, 6.0 mmol) and 4-nitro phenyl chloroformate (564 mg, 2.8 mmol). After 5 minutes reaction was allowed to warm to rt. After 1 h at rt reaction was charged with methyl amine (2N in THF, 10 ml, 20 mmol) and stirred 16 h. Reaction was then concentrated and purified by silica gel chromatography methanol in dcm 0-12% to provide EIDD-3372 (70 mg, 11% yield).

### Example 129. General Procedure for Synthesis of Disclosed 5′ Thiocarbaamate Analogs

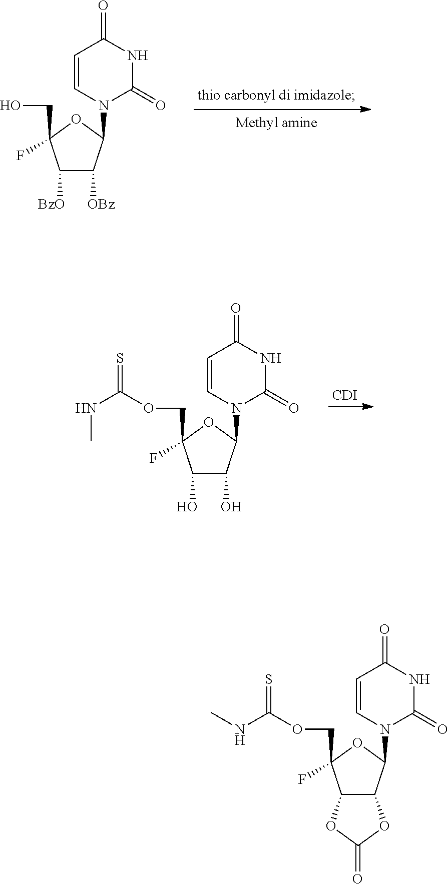

To a solution of bis benzoyl 4′ fluoro uridine (1 eq) in THF (0.2M) was added triethyl amine (3 eq) and thio carbonyl di imidazole (1.4 eq). After 2 h at rt reaction was charged with methyl amine (2N in THF, 10 eq) and stirred 16 h. Reaction was then concentrated and purified by silica gel chromatography methanol in DCM 0-12% to provide thiocarbamate. To a stirred solution of thiocarbamate (1 eq) in DCM (0.2M) was added CDI (1.2 eq). After 3 h reaction was complete by TLC and was concentrated and purified by silica gel chromatography 0-20% acetonitrile in DCM to provide 2′,3′ cyclic carbonate 5′ thiocarbamate.

### Example 130. Synthesis of EIDD-3374

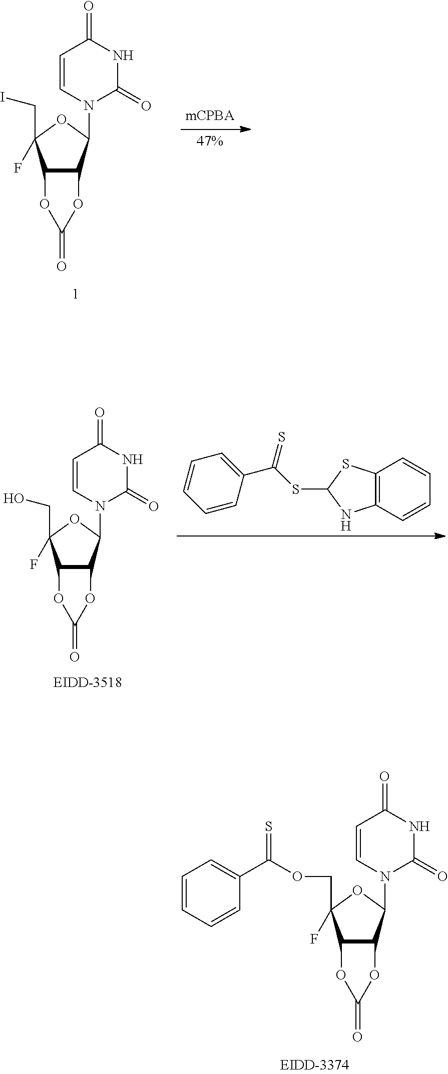

To a 55% aq. tetrabutylammonium hydroxide (10 mL) buffered to pH 4 with trifluoroacetic acid (˜1.6 mL) was added iodide 1(1.7 g, 4.27 mmol) in DCM (43 mL) and 3-chloroperbenzoic acid (3.83 g, 17.08 mmol) sequentially. After 18 h reaction was cooled to 0° C. and charged with sodium thiosulfate (30 mL) and brine (100 mL). After 30 minutes reaction was extracted with ethyl acetate (3×150 mL). The combined organics were washed with brine (50 mL) dried and concentrated. Silica gel chromatography 25-75% ethyl acetate in hexanes provided an impure fraction that was further purified by reverse phase C18 5-95% acetonitrile in water to provide EIDD-3518 (580 mg, 47%).

Thio carbonyl reagent was prepared (Bull. Korean Chem. Soc. 2002, 1029-1030).

To a solution of 1,3 benzothiazole; methyl benzenecarbodithiolate (274 mg, 0.9 mmol) in THF (3 mL) was added EIDD-3518 (200 mg, 0.69 mmol) and imidazole (56 mg, 0.83 mmol) sequentially. Reaction was heated to 55° C. for 16 h, then cooled and concentrated on celite. Celite embedded with reaction was dry loaded on a silica gel pad and product was purified by elution 0-80% ethyl acetate in hexanes to provide EIDD-3374 (90 mg, 32%). 1H NMR (400 MHz, CDCl3) o 10.00 (s, IH), 8.22 (d, J=7.3 Hz, 2H), 7.57 (t, J=7.4 Hz, IH), 7.42 (dd, J=8.3, 7 0.3 Hz, 2H), 7.34-7 0.27 (m, I H), 5.87 (dd, J=8.0, 1.5 Hz, I H), 5.83 (s, I H), 5.63-5.51 (m, 2H), 4.94 (dd, J=12.3, 9.4 Hz, IH), 4.79 (dd, J=20.1, 12.3 Hz, IH).

### Example 131. Synthesis of EIDD-3030

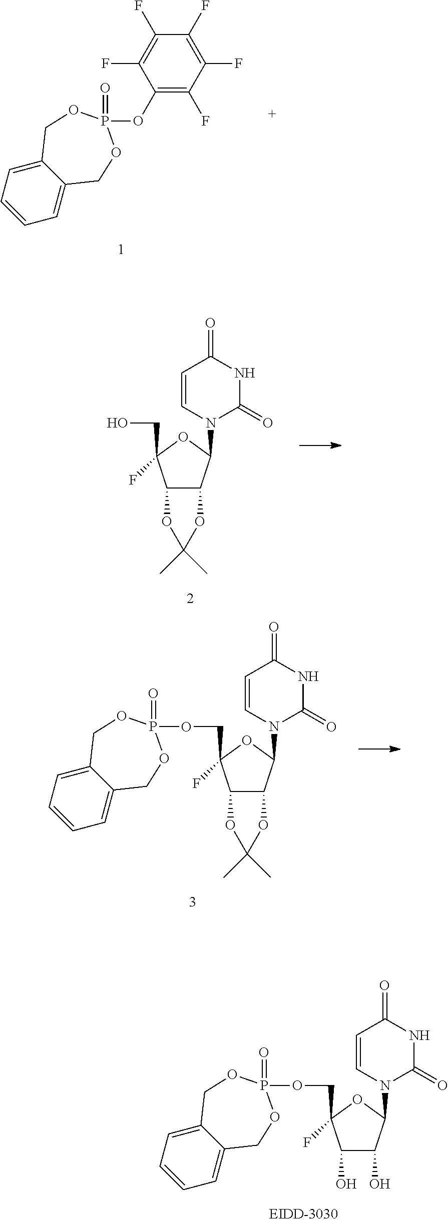 [SMILES: O=P1(Oc2c(F)c(F)c(F)c(F)c2F)OCc2ccccc2CO1 ; CC1(C)O[C@H]2[C@H](n3ccc(=O)[nH]c3=O)O[C@](F)(CO)[C@H]2O1 ; CC1(C)O[C@H]2[C@H](n3ccc(=O)[nH]c3=O)O[C@](F)(COP3(=O)OCc4ccccc4CO3)[C@H]2O1 ; O=c1ccn([C@@H]2O[C@](F)(COP3(=O)OCc4ccccc4CO3)[C@@H](O)[C@H]2O)c(=O)[nH]1]

Reagent 1: A flame-dried 500 mL round bottom flask was charged with 150 mL of anhydrous THF and phosphorus oxychloride (2.97 mL, 31.85 mmol). While under argon, the solution was cooled to −78° C. and then treated dropwise over a 30 min period via syringe pump with a mixture of [2-(hydroxymethyl)phenyl]methanol (4.00 g, 28.95 mmol) and triethylamine (8.47 mL, 60.8 mmol) in 50 mL of anhydrous THF. The mixture thus formed was allowed to warm to rt and stir at rt for 3 h. The white mixture was cooled with an ice-bath to 0° C. and then treated dropwise over a 20 min period with a THF (50 mL) solution of pentafluorophenol (4.80 g, 26.06 mmol) and Triethylamine (4.84 mL). The mixture was allowed to warm to rt slowly as the ice bath melted and continued to stir overnight at rt. In the morning reaction was then filtered. The filtrate was concentrated and purified by column chromatography over silica gel using a hexanes/ethyl acetate gradient. Isolated 1 (4.82 g, 45%) as a white solid.

Intermediate 3: Nucleoside intermediate 2 (2.12 g, 7.01 mmol) was dissolved in anhydrous THF (30 mL) and cooled with an ice-bath. After treatment with TERT-BUTYLMAGNESIUM CHLORIDE (9.11 mL, 9.11 mmol) dropwise over a 30 min period using a syringe pump, the mixture was allowed to stir with warming to rt. After 2 h, the mixture was treated with a THF (8 mL) solution of 3-(2,3,4,5,6-pentafluorophenoxy)-1,5-dihydro-2,4,3λ5-benzodioxaphosphepine 3-oxide (3.46 g, 9.46 mmol) (required gentle heating to fully dissolve). The mixture became completely homogeneous. Continued to stir at rt for 18 h. The mixture was quenched with saturated ammonium chloride solution (40 mL). Layers separated and aqueous back-extracted with methylene chloride (50 mL). Combined organic phases were dried and concentrated to a gum which was purified by column chromatography over silica gel eluting with a hexanes/ethyl acetate gradient. Material required a second purification by column chromatography over silica gel eluting methanol/DCM gradient provide intermediate 3 (2.36 g, 70%).

EIDD-3030: Intermediate 3 (500 mg, 1.03 mmol) was dissolved in methanol (10 mL) and then solid camphorsulfonic acid (240 mg, 1.03 mmol) was added in a single portion to the stirring starting material solution. After 18 h crude reaction mixture was concentrated onto celite and then purified by silica gel chromatography, 0-80% MeCN in DCM gradient to provide EIDD-3030 (135 mg, 29% yield).

### Example 132. Synthesis of EIDD-3549

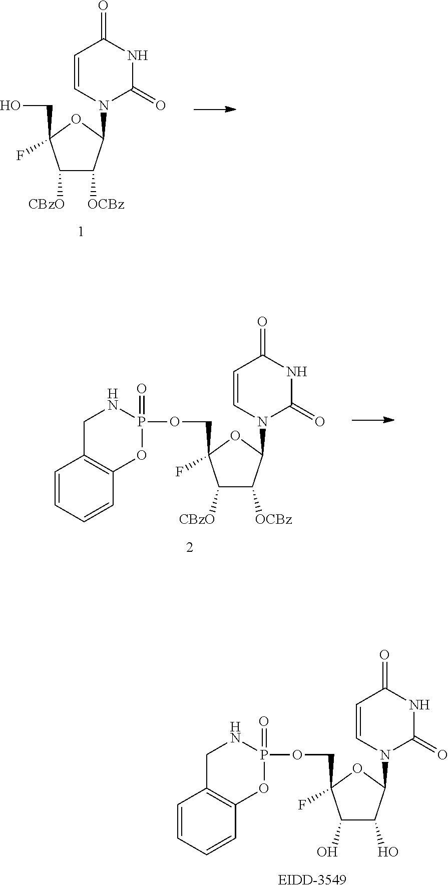

Intermediate 2: Into a solution of 2-(aminomethylphenol) (370 mg, 3.00 mmol) and TEA (836 μL, 6.00 mmol) in dry THF (15 mL) was added POCl3 (274 μL, 3.00 mmol) dropwise at 0° C. under N2. After stirring for 2 h at rt, the rxn mixture was centrifuged and the supernatant was immediately used for the next step. Into a solution of 1 (795 mg, 1.50 mmol) in dry THF (15 mL) was added tBuMgCl (1.0 M in THF) (3.00 mL, 3.00 mmol) dropwise at 0° C. under N2. After stirring for 1 h at rt, the phosphorylation reagent was added 0° C. under N2. The reaction mixture was stirred for 3 h at rt and then quenched with half sat NH4Cl aq, extracted with EtOAc 3×100 mL, dried over MgSO₄, and concentrated in vacuo. The crude was purified on a SiO2 column chromatography (Hex/EtOAc=7/3 to 3/7) to afford 2 as a white solid (737 mg, 70%).

EIDD-3549: A mixture of Intermediate 2 (737 mg, 1.06 mmol) and 10% Pd/C (112 mg, 0.106 mmol) in MeOH/DCM (3/2) (25 mL) was stirred for 30 min at rt under hydrogen (H₂ balloon), and then filtered through Celite. The filtrate was concentrated in vacuo and was then purified on a SiO2 column chromatography (DCM/MeOH=100/0 to 90/10), afforded EIDD-3549 as a white solid (317 mg, 70%). The diastereomeric ratio of TM was ˜55:45 by NMR.

### Example 133. Synthesis of EIDD-3610

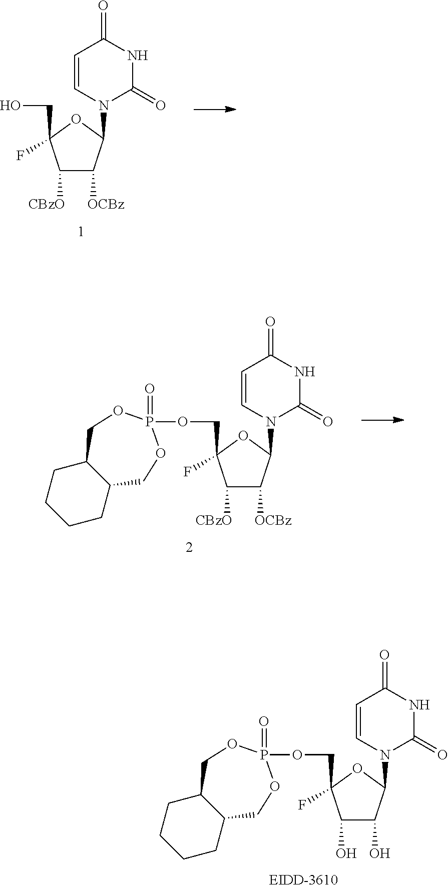

Intermediate 2: Into a solution of [(1R,2R)-2-(hydroxymethyl)cyclohexyl]methanol (652.46 mg, 4.52 mmol) and TEA (1.26 mL, 9.05 mmol) in dry THF (13 mL) was added POCl3 (420 μL, 4.6 mmol) dropwise at 0° C. under N2. After stirring for 2 h at rt, the rxn mixture was filtered and was immediately used for the next step. Into a solution of 1 (1.2 g, 2.26 mmol) in dry THF (13 mL) was added tBuMgCl (1.0 M in THF) (4.5 mL, 4.5 mmol) dropwise at 0° C. under N2. After stirring for 1 h at rt, the phosphorylation reagent was added 0° C. under N2. The reaction mixture was stirred for 3 h at rt and then quenched with sat NH4Cl aq, extracted with EtOAc 3×100 mL, dried over MgSO4, and concentrated in vacuo. The crude was purified on a SiO2 column chromatography (Hex/EtOAc=7/3 to 3/7) to afford 2 as a white solid (670 mg, 0.935 mmol, 41%).

EIDD-3610: A mixture of Intermediate 2 (650 mg, 0.91 mmol) and 10% Pd/C (104 mg) in MeOH/DCM (1:2) (12 mL) was stirred for 2 h at rt under hydrogen (H2 balloon), and then filtered through Celite. The filtrate was concentrated in vacuo and was then purified on a SiO2 column chromatography (DCM/MeOH=100/0 to 90/10), afforded EIDD-3610 as a white solid (367 mg, 89%).

¹H NMR (400 MHz, DMSO) δ 11.49 (d, J=2.2 Hz, 1H), 7.66 (d, J=8.2 Hz, 1H), 5.96 (d, J=2.9 Hz, 1H), 5.78-5.73 (m, 1H), 5.67 (dd, J=8.0, 2.2 Hz, 1H), 5.39 (d, J=8.7 Hz, 1H), 4.34 (ddd, J=17.7, 8.6, 6.7 Hz, 1H), 4.26 (ddd, J=6.8, 5.2, 2.9 Hz, 1H), 4.23-4.07 (m, 2H), 4.00-3.73 (m, 4H), 1.69 (d, J=9.0 Hz, 2H), 1.51 (d, J=10.2 Hz, 4H), 1.20 (q, J=11.3 Hz, 2H), 0.99-0.78 (m, 2H).

### Example 134. Synthesis of EIDD-3617

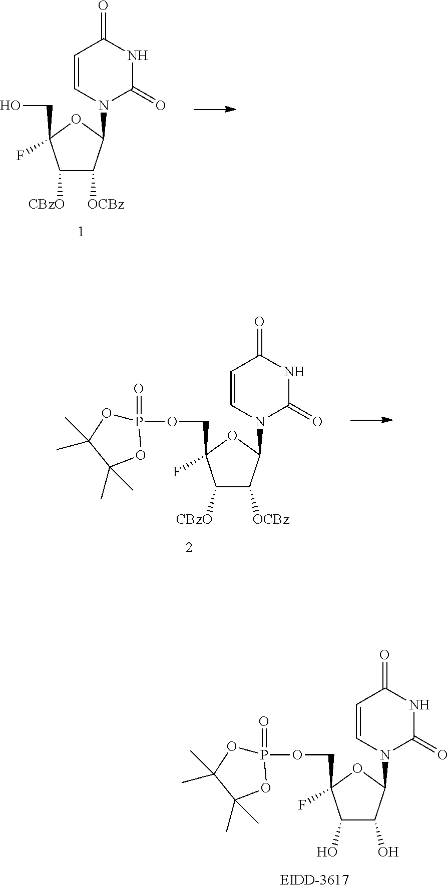

A solution of the mixture of Intermediate 1 (360.00 mg, 0.68 mmol) was dissolved in DCM (6.7 mL) and then cooled to OC. The mixture was treated dropwise with N,N-Diisopropylethylamine (0.24 mL, 1.36 mmol) followed by dropwise addition of 2-chloro-4,4,5,5-tetramethyl-1,3,2-dioxaphospholane (0.16 mL, 1.02 mmol) at 7:44 am. The mixture continued to stir at OC. At 10:00 am allowed to warm to rt. TLC (EA) at 11:30 am showed no remaining starting material. The mixture was cooled with ice bath and treated dropwise with (TBHP) in decane (0.25 mL, 1.36 mmol) at 12 pm. At 2:00 pm complete by tlc (ethyl acetate).

Reaction was concentrated in vacuo and purified by silica gel chromatography ethyl acetate provided intermediate 2 (350 mg, 83%) A mixture of Intermediate 2 (350 mg, 0.505 mmol) and 10% Pd/C (80 mg) in isopropanol, Ethyl acetate (1:10) (5.5 mL) was stirred for 1 h at rt under hydrogen (H₂ balloon), and then filtered through Celite. The filtrate was concentrated in vacuo to afforded EIDD-3617 as a white solid (189 mg, 61%).

### Example 135. Synthesis of EIDD-3639

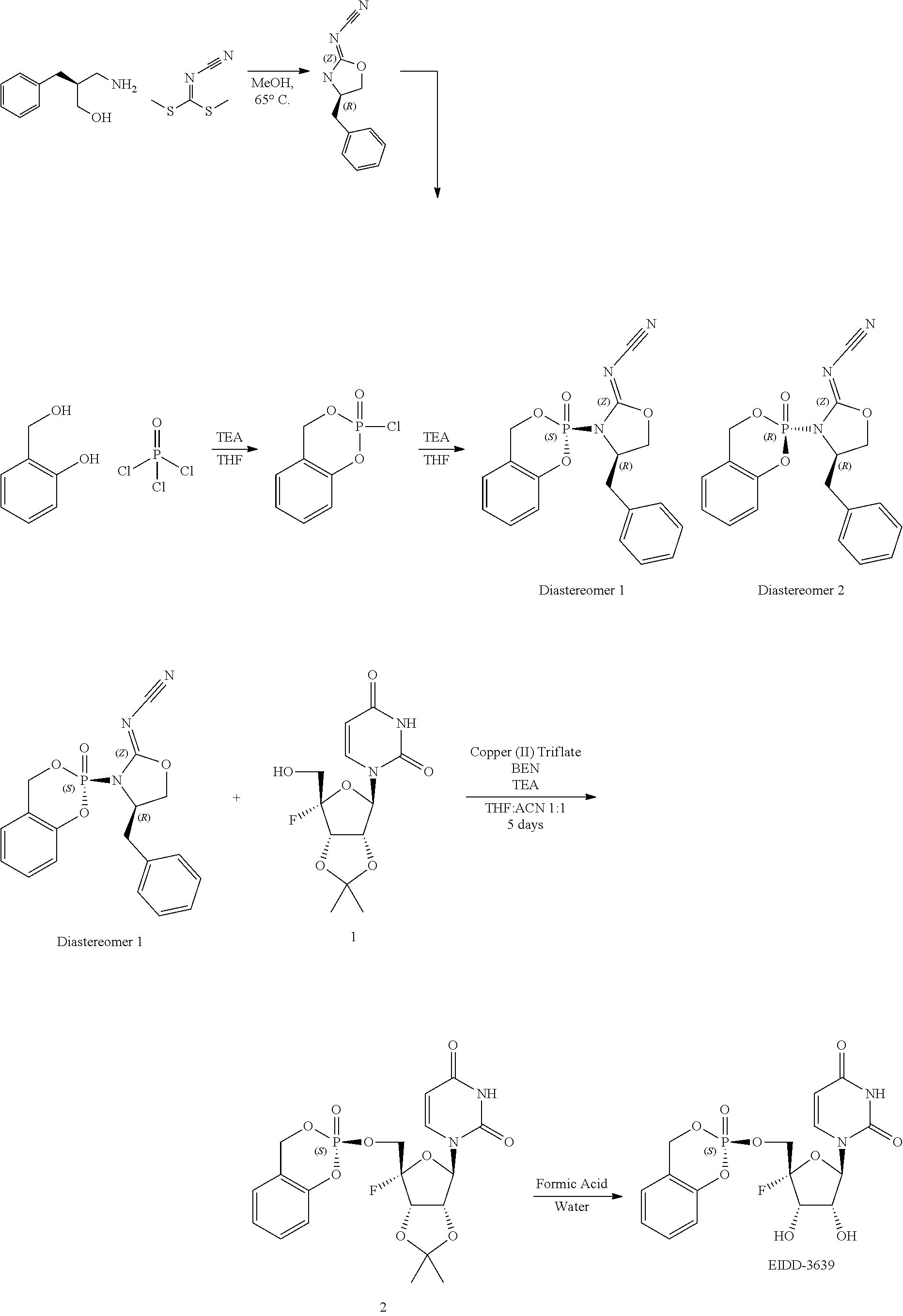

To a solution of diastereomer 1 (1 g, 2.71 mmol) (prepared based on J. Med. Chem. 2012, 55, 7245-7252) in MeCN (60 mL) and THF (60 mL) under argon was added copper triflate (979.29 mg, 2.71 mmol) and N-(2-benzamidoethyl)benzamide (726.52 mg, 2.71 mmol). In another flask charged with 1-[(3aS,4S,6R,6aR)-4-fluoro-4-(hydroxymethyl)-2,2-dimethyl-6,6a-dihydro-3aH-furo[3,4-d][1,3]dioxol-6-yl]pyrimidine-2,4-dione 1 (2 g, 6.77 mmol) in MeCN (60 mL) and THF (60 mL) was added Et₃N (0.94 mL, 6.77 mmol). After 30 min of stirring, the first solution was added, and the mixture was stirred at rt for 5 days. The mixture was diluted with sat aq NH₄Cl then extracted with DCM. The organic layers were combined, dried over Na₂SO₄, filtered, and concentrated under pressure. The crude material was purified by SiO₂ column chromatography eluting with 0-10% MeOH in DCM. The product was repurified by SiO₂ column chromatography eluting with 0-70% EtOAc in hexanes to yield 1-[(3aS,4S,6R,6aR)-4-fluoro-2,2-dimethyl-4-[[(2R)-2-oxo-4H-1,3,2λ5-benzodioxaphosphinin-2-yl]oxymethyl]-6,6a-dihydro-3aH-furo[3,4-d][1,3]dioxol-6-yl]pyrimidine-2,4-dione 2 (310 mg, 24.3% yield) as a white solid.

1-[(3aS,4S,6R,6aR)-4-fluoro-2,2-dimethyl-4-[[(2R)-2-oxo-4H-1,3,2λ5-benzodioxaphosphinin-2-yl]oxymethyl]-6,6a-dihydro-3aH-furo[3,4-d][1,3]dioxol-6-yl]pyrimidine-2,4-dione 2 (310 mg, 0.66 mmol) was dissolved in 80% formic acid (25 mL, 0.66 mmol) and stirred at rt with TLC monitoring. The solution was concentrated under pressure and purified by SiO₂ column chromatography eluting with 0-15% MeOH in DCM to yield 1-[(2R,3R,4S,5S)-5-fluoro-3,4-dihydroxy-5-[[(2R)-2-oxo-4H-1,3,2λ5-benzodioxaphosphinin-2-yl]oxymethyl]tetrahydrofuran-2-yl]pyrimidine-2,4-dione EIDD-3639 (171.3 mg, 60.4% yield) as a white solid. ¹H NMR (400 MHz, CD₃OD) δ 7.45 (d, J=8.1 Hz, 1H), 7.40-7.30 (m, 1H), 7.30-7.15 (m, 2H), 7.09 (ddd, J=8.2, 1.0, 0.5 Hz, 1H), 5.87 (d, J=2.1 Hz, 1H), 5.53 (dd, J=8.3, 7.2 Hz, 2H), 5.49-5.38 (m, 1H), 4.49 (dd, J=18.9, 6.9 Hz, 1H), 4.45-4.33 (m, 3H). ¹³C NMR (101 MHz, CD₃OD) δ 165.86, 151.56, 151.11, 151.05, 143.98, 131.06, 126.86, 125.97, 122.19, 122.09, 119.55, 119.46, 117.51, 117.42, 115.21, 115.12, 103.21, 97.00, 72.32, 71.32, 71.11, 70.48, 70.41, 66.78, 66.37, 66.31. ¹⁹F NMR (376 MHz, CD₃OD) δ −124.67, −124.69, −124.71, −124.72, −124.74, −124.76. ³¹P NMR (162 MHz, CD₃OD) δ −9.68. LCMS shows M/z=453 (M+Na+) and 429 (M−H).

### Example 135. Synthesis of EIDD-3640

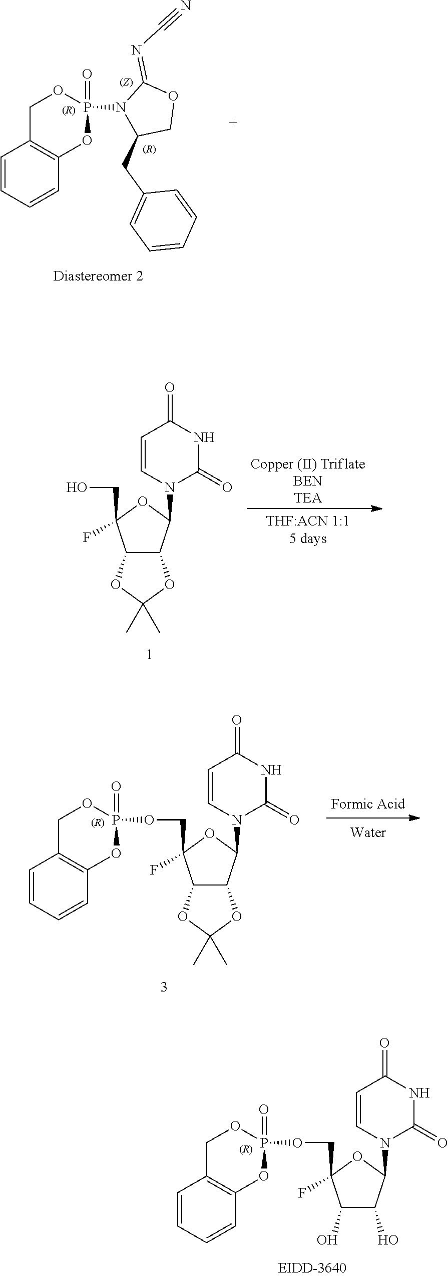

To a solution of diastereomer 2 (1 g, 2.71 mmol) (prepared based on J. Med. Chem. 2012, 55, 7245-7252) (500 mg, 1.35 mmol) in MeCN (15 mL) and THF (15 mL) under argon was added copper triflate (489.64 mg, 1.35 mmol) and N-(2-benzamidoethyl) benzamide (363.26 mg, 1.35 mmol). In another flask charged with 1-[(3aS,4S,6R,6aR)-4-fluoro-4-(hydroxymethyl)-2,2-dimethyl-6,6a-dihydro-3aH-furo[3,4-d][1,3]dioxol-6-yl]pyrimidine-2,4-dione 1 (1 g, 3.38 mmol) in MeCN (15 mL) and THF (15 mL) was added Et₃N (0.47 mL, 3.38 mmol). After 30 min of stirring, the first solution was added, and the mixture was stirred at rt for 5 days. The mixture was diluted with sat aq NH₄Cl then extracted with DCM. The organics were combined, dried over Na₂SO₄, filtered, and concentrated under pressure. The crude material was purified by SiO₂ column chromatography eluting with 0-10% MeOH in DCM. The product was repurified by SiO₂ column chromatography eluting with 0-70% EtOAc in hexanes to yield 1-[(3aS,4S,6R,6aR)-4-fluoro-2,2-dimethyl-4-[[(2S)-2-oxo-4H-1,3,2λ5-benzodioxaphosphinin-2-yl]oxymethyl]-6,6a-dihydro-3aH-furo[3,4-d][1,3]dioxol-6-yl]pyrimidine-2,4-dione 3 (126.9 mg, 19.9% yield) as a white solid.

1-[(3aS,4S,6R,6aR)-4-fluoro-2,2-dimethyl-4-[[(2S)-2-oxo-4H-1,3,2λ5-benzodioxaphosphinin-2-yl]oxymethyl]-6,6a-dihydro-3aH-furo[3,4-d][1,3]dioxol-6-yl]pyrimidine-2,4-dione 3 (126.9 mg, 0.27 mmol) was dissolved in 80% formic acid (25.00 mL, 0.66 mmol) and stirred for 4 h. The reaction was concentrated under pressure and purified by SiO₂ column chromatography eluting with 0-15% MeOH in DCM to yield 1-[(2R,3R,4S,5S)-5-fluoro-3,4-dihydroxy-5-[[(2R)-2-oxo-4H-1,3,2λ5-benzodioxaphosphinin-2-yl]oxymethyl]tetrahydrofuran-2-yl]pyrimidine-2,4-dione EIDD-3640 (74.1 mg, 63.83% yield) as a white solid. ¹H NMR (400 MHz, CD₃OD) δ 7.45 (d, J=8.1 Hz, 1H), 7.38-7.27 (m, 1H), 7.23-7.12 (m, 2H), 7.07 (dd, J=8.2, 1.0 Hz, 1H), 5.76 (d, J=2.1 Hz, 1H), 5.64-5.54 (m, 2H), 5.44 (dd, J=19.5, 14.3 Hz, 1H), 4.57 (dd, J=19.4, 6.9 Hz, 1H), 4.44-4.28 (m, 3H). ¹³C NMR (101 MHz, CD₃OD) δ 165.90, 151.47, 151.03, 150.96, 144.50, 130.93, 126.92, 125.83, 122.11, 122.01, 119.55, 119.46, 117.52, 117.43, 115.15, 103.15, 97.71, 72.02, 71.11, 70.91, 70.72, 70.65, 66.29, 65.84. ³¹P NMR (162 MHz, CD₃OD) δ −9.78. ¹⁹F NMR (376 MHz, CD₃OD) δ −124.37, −124.39, −124.41, −124.42, −124.44, −124.46. LCMS shows M/z=453 (M+Na+) and 429 (M−H).

### Example 136. Drug Metabolism: General Protocol for Stability of Test Article in Simulated Gastric Fluid

Test article, e.g., EIDD-2749 prodrug, was incubated in triplicate in SGF without pepsin, pH 1, and at 37° C. and at a concentration of the prodrugs of 10.0 μM. Incubations were performed in 2-mL LC autosampler vials. SGF (990 μL) was added to the vials. Vials were placed in the LC autosampler set at 37° C. for a minimum of 15 minutes to equilibrate the temperature of the SGF. Each replicate was removed from the autosampler (one at a time, 5 minutes apart) and 5 μL of an internal standard (2 mM 5-fluoruracil in water) and 5 μL of a 2 mM solution of each prodrug in DMSO were added to the SGFs, mixed well by vortexing and placed back in the autosampler.

An injection of the first replicate was made using the analytical LC-MS/MS method (refer to the next section). The total run time of the method was 5.0 minutes, including needle and injection path cleaning. Replicates were staggered 5 min apart so when they were reinjected, 15 minutes had elapsed. Injections were made at times: 0, 15, 30, 45, 60, 75, 90, 105, and 120 minutes, which is more than the residence time of a drug in the stomach.

Concentrations of prodrugs were quantitated using a one-point calibration in triplicate where the time-zero samples were assigned their theoretical concentration of 10.0 μM. Quantitation of analytes was performed by means of the ratio of peak area counts of the analyte of interest to the internal standard using the SCIEX OS v 3.0.0.0.3339 and the M4Q algorithm. Production of the nucleoside EIDD-2749 and metabolites (uracil, M1, the monophosphate or any intermediate species) were not followed in this screening study based on previous experience with similar compounds in SGF.

Half-lives (t_{1/2}) were calculated by plotting the natural logarithm of the analyte concentration vs. time and obtaining the slope of the line. Assuming first-order kinetics, the elimination rate constant, k, is the negative (−) of the slope of the plot (ln[μM] vs. time). Half-life (t_{1/2}) (min)=−0.693/(slope)

### Example 137. Intracellular Concentration of Representative Prodrugs after Incubation

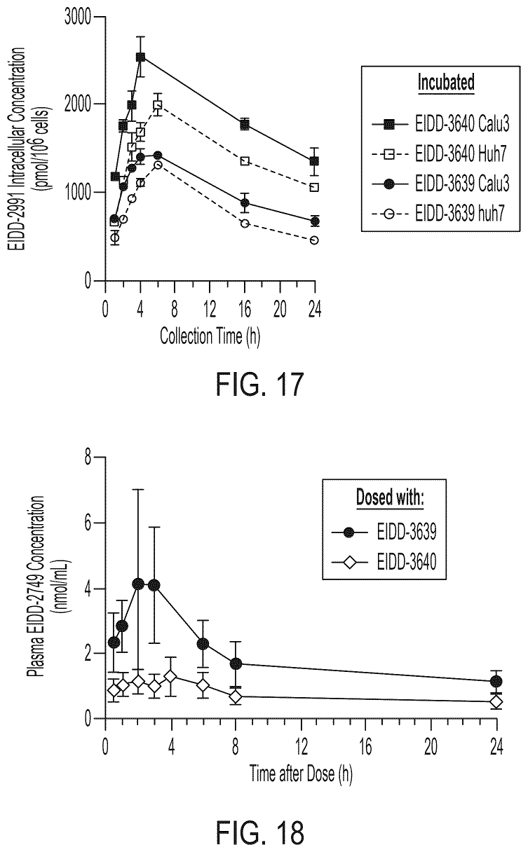

Calu-3 cells and Huh7 cells were incubated in complete DMEM cell culture media containing either EIDD-3639 or EIDD-3640 at 20 μM for 1, 2, 3, 4, 6, 16 and 24 hours. Following incubation, cells were washed with cold PBS twice and cells were extracted with extraction solution. Intracellular concentrations of the 5′-triphosphate metabolite, EIDD-2991, were determined in the extracts by LC-MS/MS. Intracellular concentrations of EIDD-2991 in each study are shown in FIG. 17. Incubation with EIDD-3640 generated higher intracellular concentrations of EIDD-2991 compared to incubation with EIDD-3639 in each cell line.

### Example 138. Mouse Plasma Concentrations and Pharmacokinetic Parameters for EIDD-2749 after a Single Oral Dose of EIDD-3639 or EIDD-3640

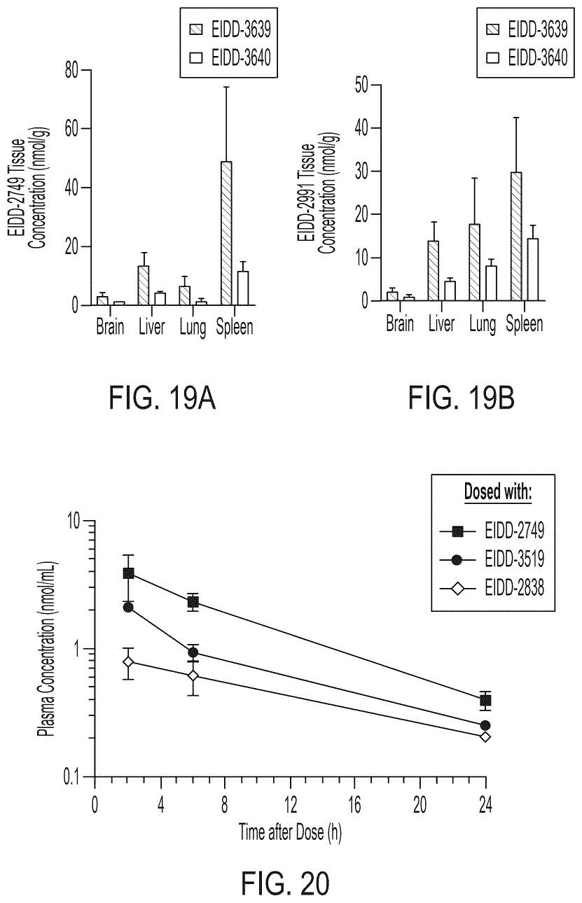

Female C57/BL6 mice were dosed with a single oral dose of EIDD-3639 or EIDD-3640 at 16.5 mg/kg. Blood was collected up to 24 hours post-dose (hpd) from four mice per test article per timepoint into lithium-heparin containing microtainer tubes, and plasmas were prepared and stored frozen at −80° C. Plasma concentrations of the nucleoside metabolite, EIDD-2749, were determined by a qualified LC-MS/MS method. Mouse organs were collected at 3 hours post-dose and snap-frozen. Tissue levels of EIDD-2749 and EIDD-2991 were analyzed by a qualified LC-MS/MS method. Plasma concentrations of EIDD-2749 are shown in FIG. 18 and tissue concentrations of EIDD-2749 and EIDD-2991 are shown in FIG. 19. EIDD-3639 provides higher plasma concentrations of EIDD-2749 and higher tissue concentrations of both EIDD-2749 and EIDD-2991 in tissues compared to EIDD-3640. Data were analyzed and Tₘₐₓ, Cₘₐₓ, AUC_{inf}, and t_{1/2} obtained from the data are shown in the table immediately below.

| | Tₘₐₓ | Cₘₐₓ | AUC_{inf} | t_{1/2} |
|---|---|---|---|---|
| Dosed with | (h) | (nmol/mL) | (h*nmol/mL) | (h) |
| EIDD-3639 | 2 | 4.13 ± 2.94 | 75.2 | 20.2 |
| EIDD-3640 | 4 | 1.29 ± 0.587 | 35.3 | 23.9 |

### Example 139. Mouse Plasma Concentrations and Pharmacokinetic Parameters for EIDD-2749 after a Single Oral Dose of EIDD-2749, EIDD-3519, or EIDD-2838

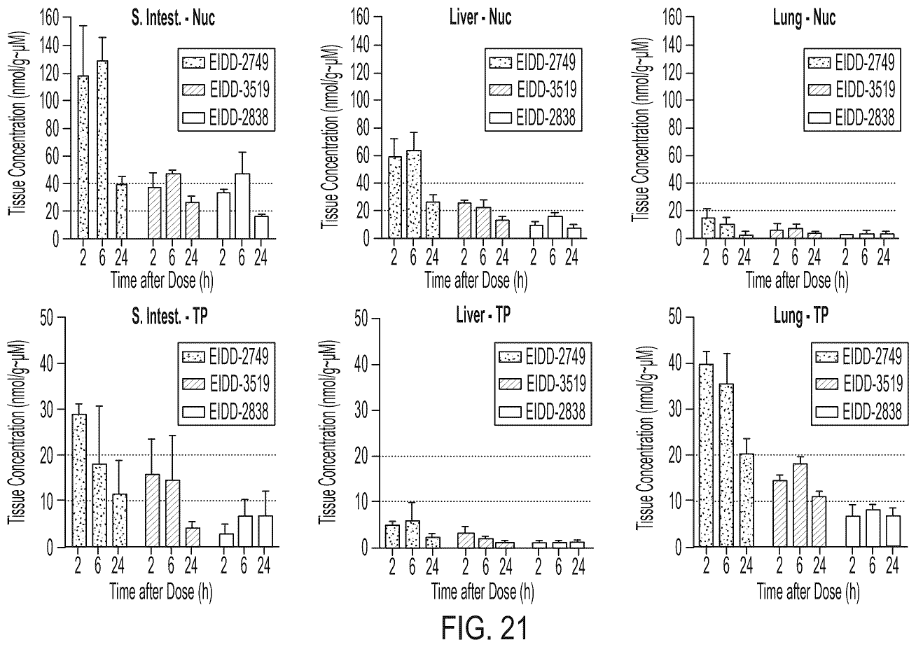

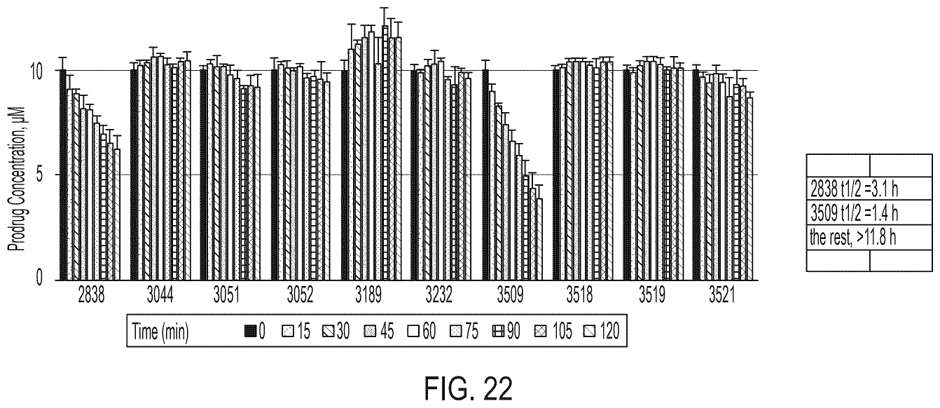

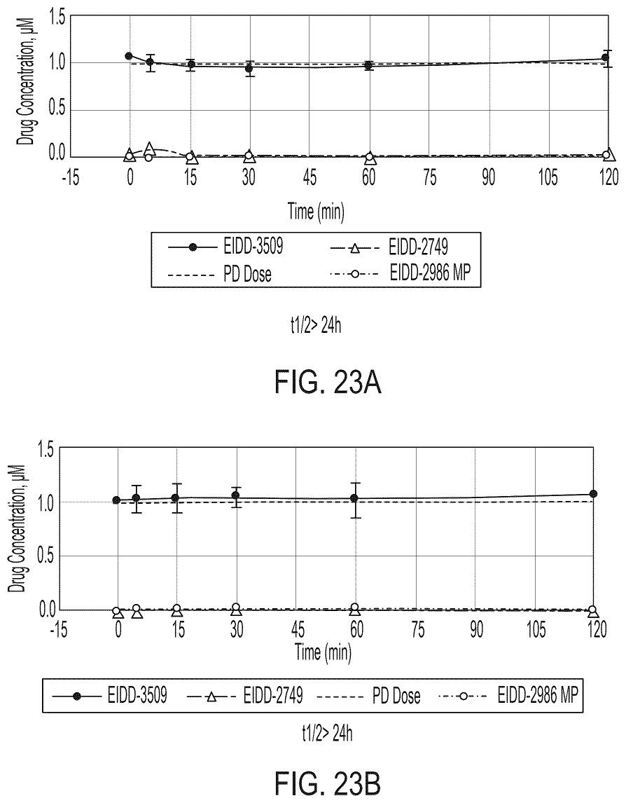

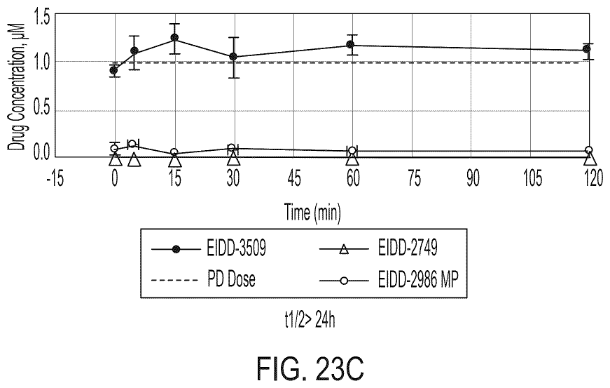

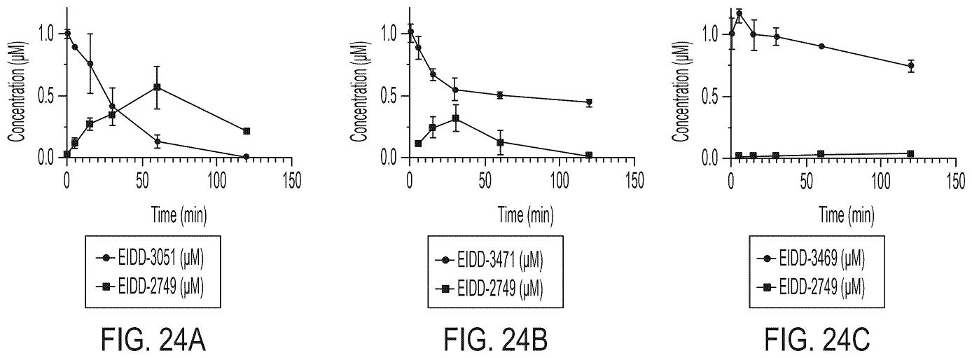

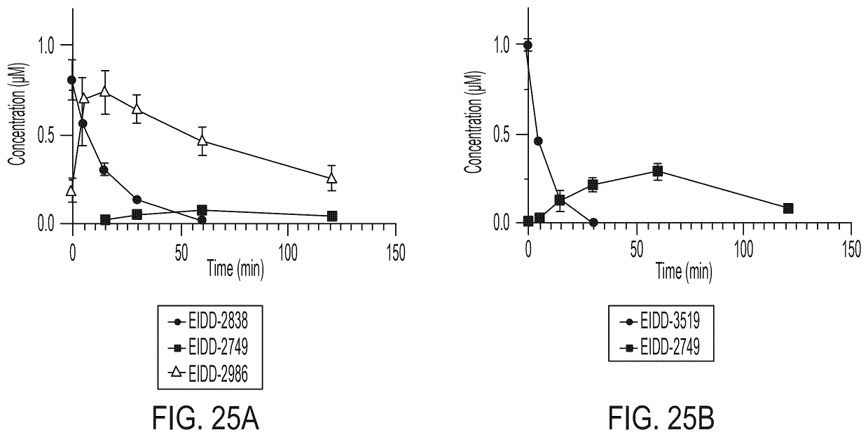

Female C57/BL6 mice were dosed with a single oral dose of EIDD-2749 at 15 mg/kg or a single dose of a prodrug, EIDD-3519 or EIDD 2838, at 25 mg/kg, an approximately equimolar dose. Blood and tissues were collected at 2, 6, and 24 hours post-dose (hpd) from three mice per compound per timepoint. Blood was collected into lithium-heparin containing microtainer tubes, and plasmas were prepared and stored frozen at −80° C. before analysis. Tissues were snap-frozen in liquid nitrogen immediately following collection and stored at −80° C. before analysis. Plasma concentrations of the nucleoside, EIDD-2749, and tissue concentrations of EIDD-2749 and the 5′-triphosphate metabolite, EIDD-2991, were determined by qualified LC-MS/MS methods. Plasma concentrations of EIDD-2749 after a single oral dose of EIDD-2749, EIDD-3519, or EIDD-2838 are shown in FIG. 20. EIDD-2749-dosed mice provide the highest plasma concentrations of EIDD-2749 followed by EIDD-3519 and EIDD-2838 at each timepoint, respectively. Tissue EIDD-2749 (Nuc) and EIDD-2991 levels (TP) are shown in FIG. 21. Both EIDD-3519 and EIDD-2838 decrease the exposure to EIDD-2749 and EIDD-2991 compared to EIDD-2749-dosed mice.

### Example 140. Tolerability and Efficacy Data of Representative Prodrugs in Black-6 Mice

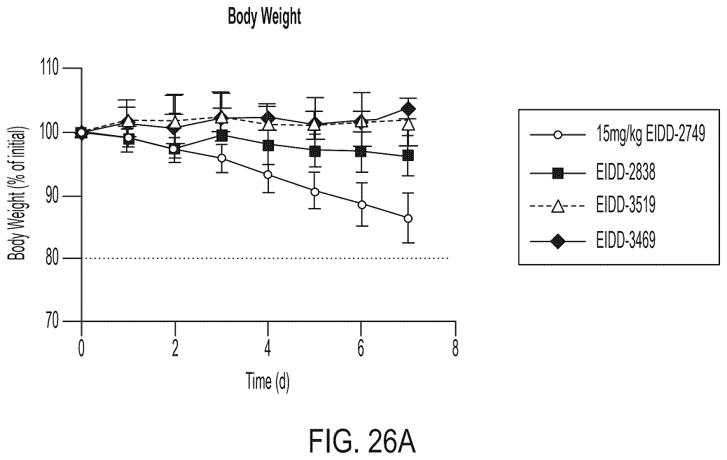

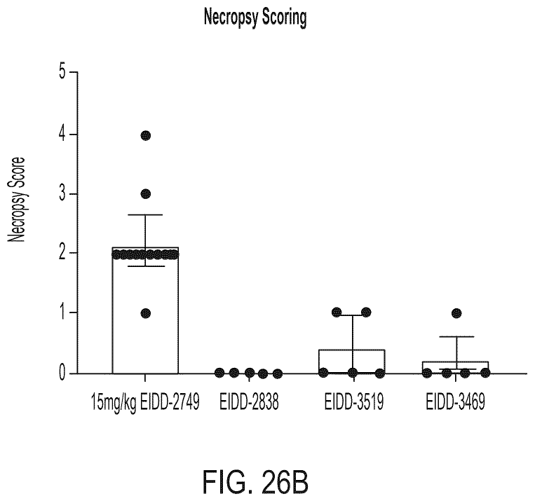

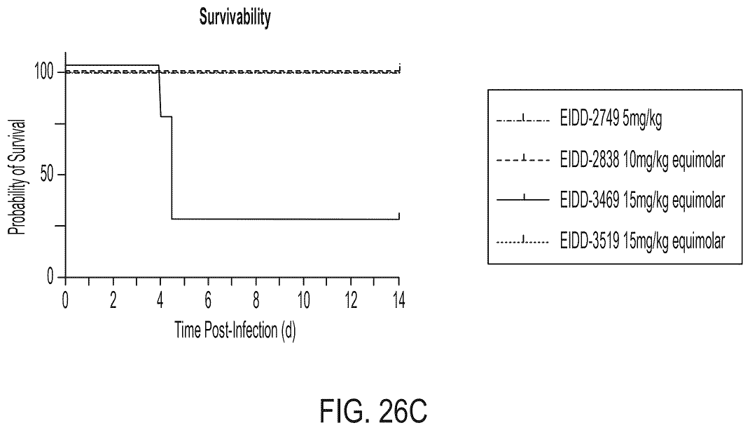

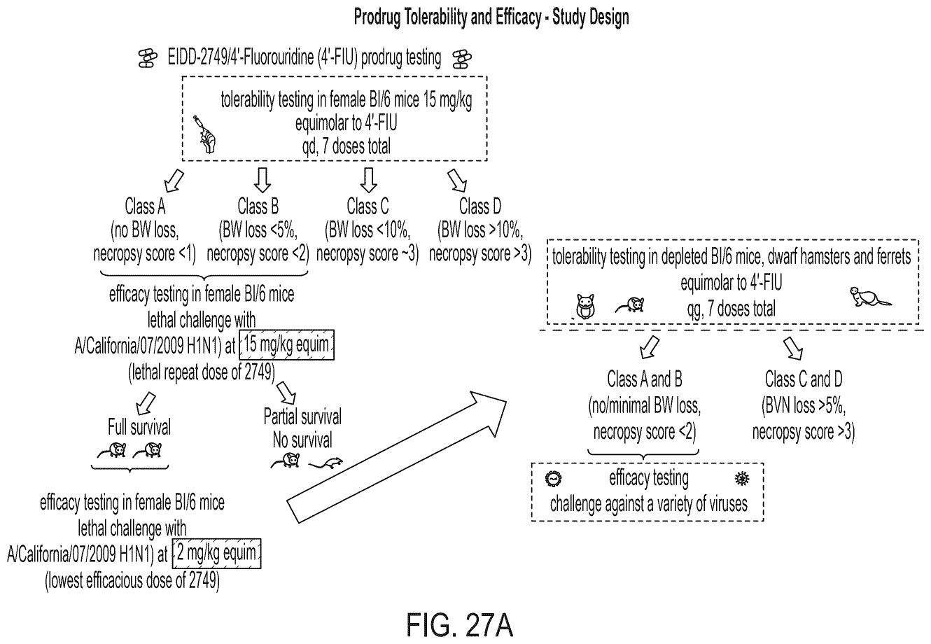

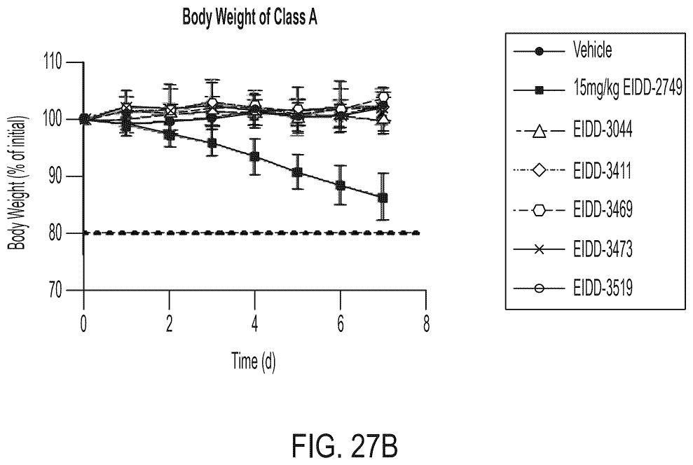

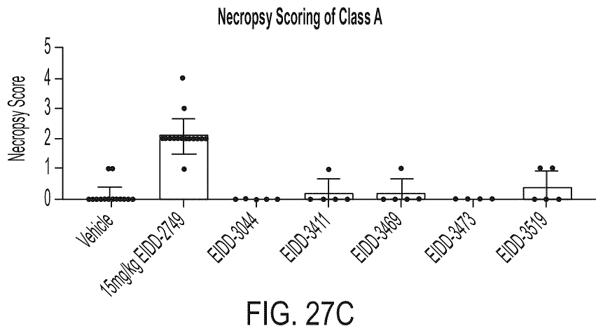

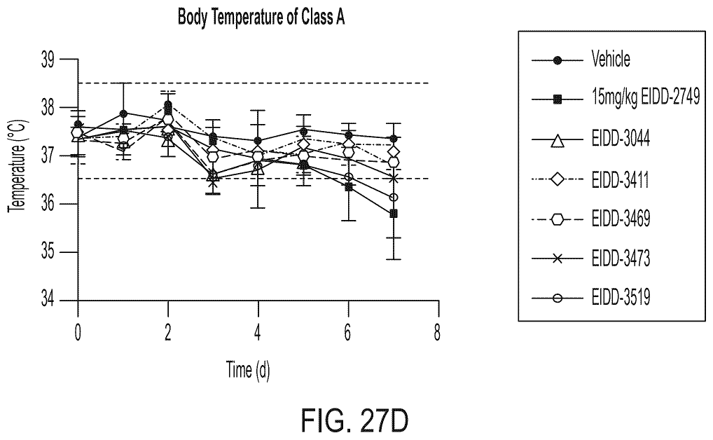

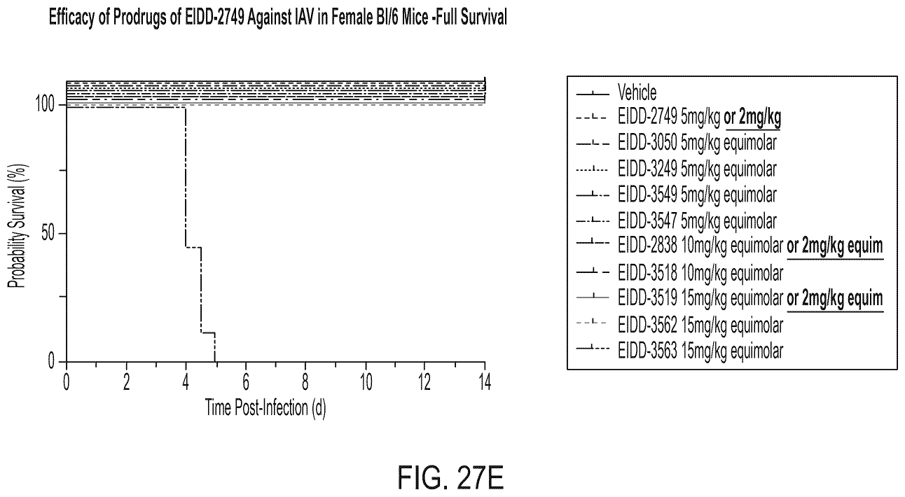

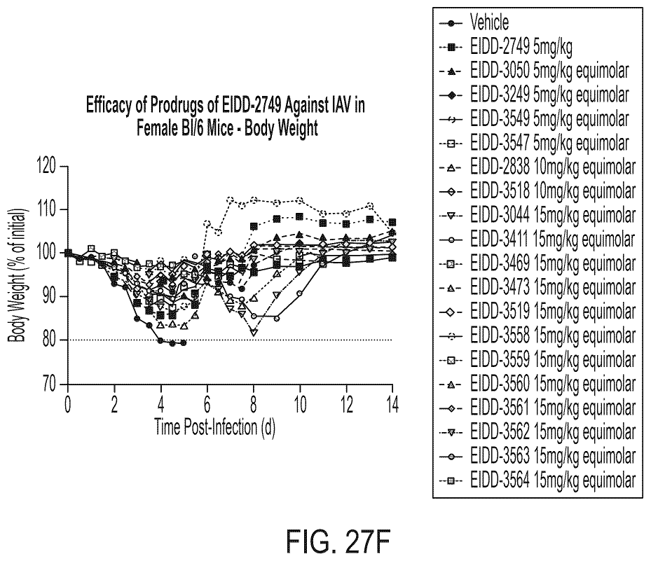

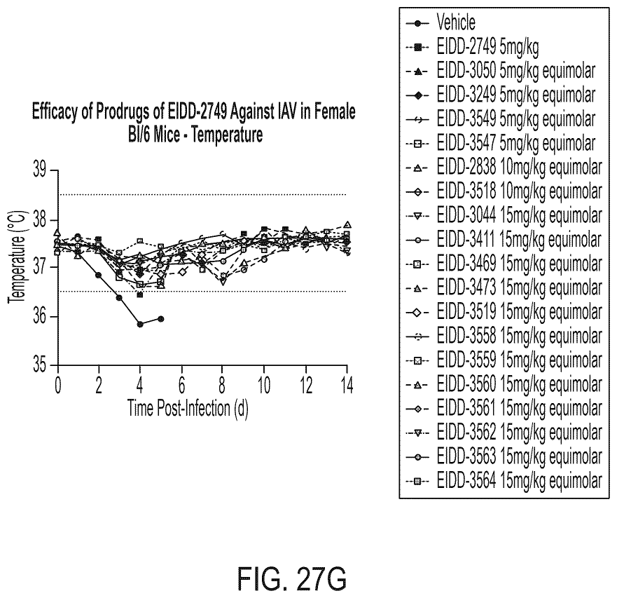

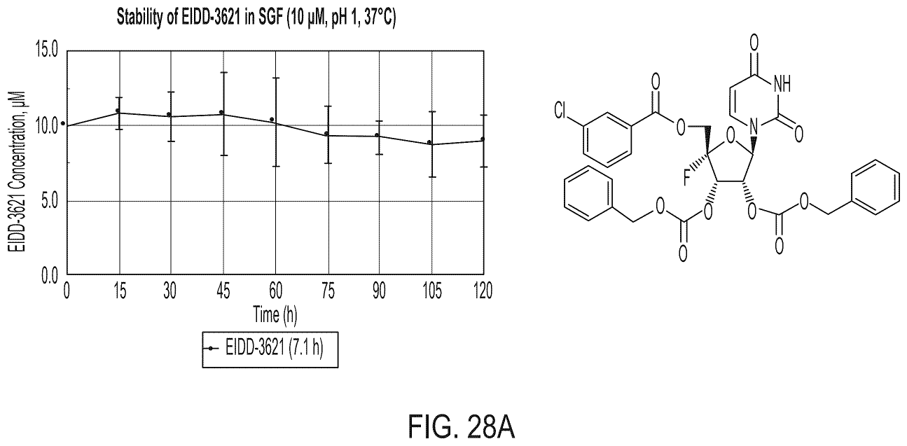

Representative data for selected compounds are provided in FIGS. 26A-26C and FIGS. 27B-27G. The tolerability and efficacy study protocol used with the compounds for which data are shown in FIGS. 26A-26C and FIGS. 27B-27G of the compounds was carried out as described in the schematic outline in FIG. 27A, briefly the indicated disclosed compounds were studied in Black-6 mice, and compared to EIDD-2749 in the same model. The compounds dosed QD, 7 days (PO), n=5, and dosed equimolar with 15 mg/kg EIDD-2749. Data were collected accordingly and are summarized in the table below. Representative data for selected drugs that are summarized in the table below are shown in FIGS. 27B-G. Similar data were obtained for all the other drugs summarized in the table below to provide the summary in the table below. The “Drug ID” or “EIDD ID” given in the table is as used elsewhere in the present disclosure and corresponds to the compound structure associated with said “EIDD” number.

| | | Tolerability | Equimolar to | | IAV | IAV Efficacy |
|---|---|---|---|---|---|---|
| | Tolerability | Necropsy | 4′-FIU for | | Efficacy | Titer |
| Drug-ID | Summary | Score | IAV Efficacy | | Survival | Reduction |
| EIDD-2749 | Reference | 2 | Ref: 5 mg/kg | | 11/11 | ND |
| | | | Ref: 2 mg/kg | | 5/5 | 3 log reduction |
| EIDD-3044 | Class A | 0 | 15 | mg/kg | 1/4 | ND |
| EIDD-3411 | Class A | 0 | 15 | mg/kg | 2/4 | ND |
| EIDD-3469 | Class A | 0 | 15 | mg/kg | 1/4 | ND |
| EIDD-3473 | Class A | 0 | 15 | mg/kg | 3/4 | ND |
| EIDD-3519 | Class A | 0 | 15 | mg/kg | 9/9 | 3 log reduction |
| | | | 2 | mg/kg | 8/8 | Sterilizing - 5 log |
| | | | | | | reduction |
| EIDD-3562 | Class A | 0 | 15 | mg/kg | 3/3 | ND |
| EIDD-3563 | Class A | 0 | 15 | mg/kg | 3/3 | ND |
| | | | 2 | mg/kg | 0/3 | ND |
| EIDD-3595 | Class A | 0 | 15 | mg/kg | 0/3 | ND |
| | Class C | 1 | 30 | mg/kg | | |
| | (30 mg/kg) | | | | | |
| EIDD-3577 | Class A | 1 | 15 | mg/kg | 1/3 | ND |
| EIDD-3588 | Class A | 1 | 15 | mg/kg | 0/3 | ND |
| EIDD-3596 | Class A | 0 | 15 | mg/kg | 0/3 | ND |
| | Class A | 0 | 30 | mg/kg | 2/3 | ND |
| | (30 mg/kg) | | | | | |
| EIDD-3587 | Class A | 1 | 15 | mg/kg | 2/3 | ND |
| EIDD-3590 | Class A | 1 | 15 | mg/kg | 2/3 | ND |
| EIDD-3589 | Class A | 1 | 15 | mg/kg | 0/3 | ND |
| EIDD-3591 | Class A | 1 | 15 | mg/kg | 2/3 | ND |
| EIDD-3592 | Class A | 1 | 15 | mg/kg | 2/3 | ND |
| EIDD-3594 | Class A | 0 | 15 | mg/kg | 0/3 | ND |
| EIDD-3567 | Class A | 1 | 15 | mg/kg | 1/3 | ND |
| EIDD-3607 | Class A | 1 | 15 | mg/kg | 3/4 | ND |
| EIDD-3610 | Class A | 0 | 15 | mg/kg | 0/4 | ND |
| EIDD-3618 | Class A | 0 | 15 | mg/kg | 2/4 | ND |
| EIDD-3579 | Class A | 1 | 15 | mg/kg | 3/4 | ND |
| EIDD-3620 | Class A | 1 | 15 | mg/kg | 1/4 | ND |
| EIDD-3619 | Class A | 1 | 15 | mg/kg | 0/4 | ND |
| EIDD-3621 | Class A | 0 | 15 | mg/kg | 3/4 | ND |
| | | | 2 | mg/kg | 0/4 | No reduction |
| EIDD-3616 | Class A | 0 | 15 | mg/kg | 3/4 | ND |
| EIDD-3030 | Class A | 0 | 15 | mg/kg | 0/4 | ND |
| EIDD-3604 | Class A | 0 | 15 | mg/kg | 0/4 | ND |
| EIDD-2838 | Class B | 0 | 10 | mg/kg | 9/9 | 3 log reduction |
| | | | 2 | mg/kg | 8/8 | Sterilizing - 5 log |
| | | | | | | reduction |
| EIDD-3518 | Class B | 1 | 10 | mg/kg | 4/4 | ND |
| EIDD-3564 | Class B | 0 | 10 | mg/kg | 10/3 | ND |
| EIDD-3617 | Class B | 2 | 10 | mg/kg | 4/4 | ND |
| | | | 2 | mg/kg | 0/4 | No reduction |
| EIDD-3050 | Class C | 2 | 5 | mg/kg | 3/3 | ND |
| EIDD-3249 | Class C | 0 | 5 | mg/kg | 4/4 | ND |
| EIDD-3549 | Class C | 1 | 5 | mg/kg | 4/4 | ND |
| EIDD-3547 | Class C | 0 | 5 | mg/kg | 4/4 | ND |
| EIDD-3581 | Class C | 1 | 5 | mg/kg | 0/3 | ND |
| EIDD-3580 | Class C | 2 | 5 | mg/kg | 2/3 | ND |
| EIDD-3593 | Class C | 1 | 5 | mg/kg | 2/3 | ND |
| EIDD-3574 | Class C | 2 | 5 | mg/kg | 1/3 | ND |
| EIDD-3048 | Class D | 3 | ND | | ND | ND |
| EIDD-3189 | Class D | 2 | ND | | ND | ND |
| EIDD-3232 | Class D | 1 | ND | | ND | ND |
| EIDD-3470 | Class D | 3 | ND | | ND | ND |
| EIDD-3472 | Class D | 2 | ND | | ND | ND |
| EIDD-3474 | Class D | 2 | ND | | ND | ND |
| EIDD-3475 | Class D | 2 | ND | | ND | ND |
| EIDD-3484 | Class D | 2 | ND | | ND | ND |
| EIDD-3539 | Class D | 2 | ND | | ND | ND |
| EIDD-3558 | ND | ND | 15 | mg/kg | 1/3 | ND |
| EIDD-3559 | ND | ND | 15 | mg/kg | 2/3 | ND |
| EIDD-3560 | ND | ND | 15 | mg/kg | 1/3 | ND |
| EIDD-3561 | ND | ND | 15 | mg/kg | 2/3 | ND |
| EIDD-3564 | ND | ND | 15 | mg/kg | 2/3 | ND |

### Example 141. Mouse Plasma Concentrations and Pharmacokinetic Parameters for EIDD-2749 after a Single Oral Dose of EIDD-2749, EIDD-3519, or EIDD-2838

Female C₅₇/BL6 mice were dosed with a single oral dose of EIDD-2749 at 15 mg/kg or a single dose of a prodrug, EIDD-3519 or EIDD 2838, at 25 mg/kg, an approximately equimolar dose. Blood and tissues were collected at 2, 6, and 24 hours post-dose (hpd) from three mice per compound per timepoint. Blood was collected into lithium-heparin containing microtainer tubes, and plasmas were prepared and stored frozen at −80° C. before analysis. Tissues were snap-frozen in liquid nitrogen immediately following collection and stored at −80° C. before analysis. Plasma concentrations of the nucleoside, EIDD-2749, and tissue concentrations of EIDD-2749 and the 5′-triphosphate metabolite, EIDD-2991, were determined by qualified LC-MS/MS methods. Plasma concentrations of EIDD-2749 after a single oral dose of EIDD-2749, EIDD-3519, or EIDD-2838 are shown in FIG. 20. EIDD-2749-dosed mice provide the highest plasma concentrations of EIDD-2749 followed by EIDD-3519 and EIDD-2838 at each timepoint, respectively. Tissue EIDD-2749 (Nuc) and EIDD-2991 levels (TP) are shown in FIG. 21. Both EIDD-3519 and EIDD-2838 decrease the exposure to EIDD-2749 and EIDD-2991 compared to EIDD-2749-dosed mice.

### Example 142. Stability of Selected Disclosed Drugs in Simulated GI Fluids and Rate of Release of EIDD-2749 from Selected Prodrugs Incubated in the Presence of Microsomes

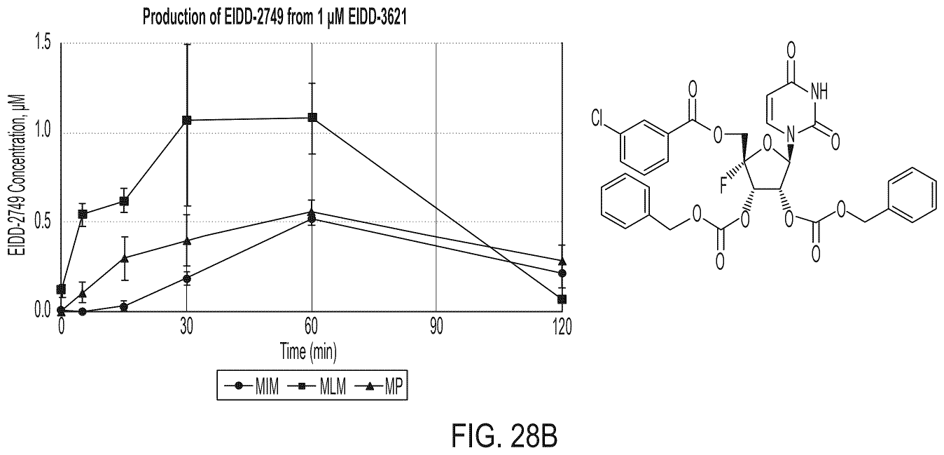

Data are given in FIG. 28A for stability of EIDD-3621 in simulated gastric fluid using the protocol as described herein above. Data are given in FIG. 28B for release of EIDD-2749 from EIDD-3621 when incubated in the presence of mouse intestinal microsomes, mouse liver microsomes, and mouse plasma using the protocols as described herein above.

### Example 143. Prophylactic Efficacy of EIDD-2749 in a Mouse Model of Tacaribe Infection

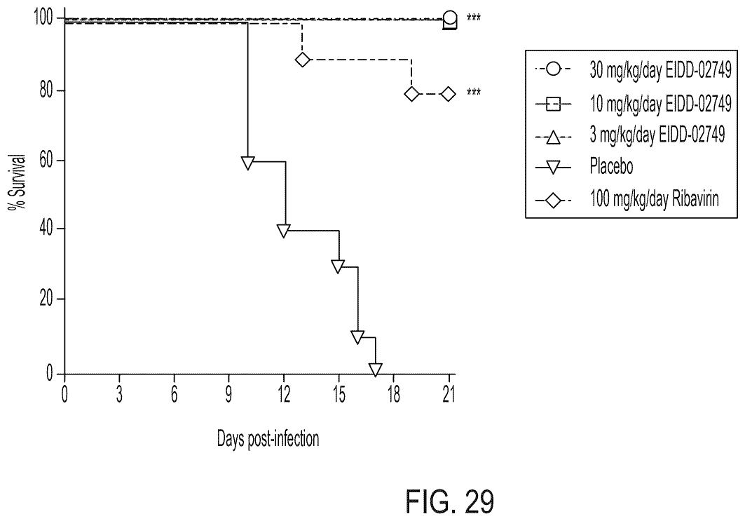

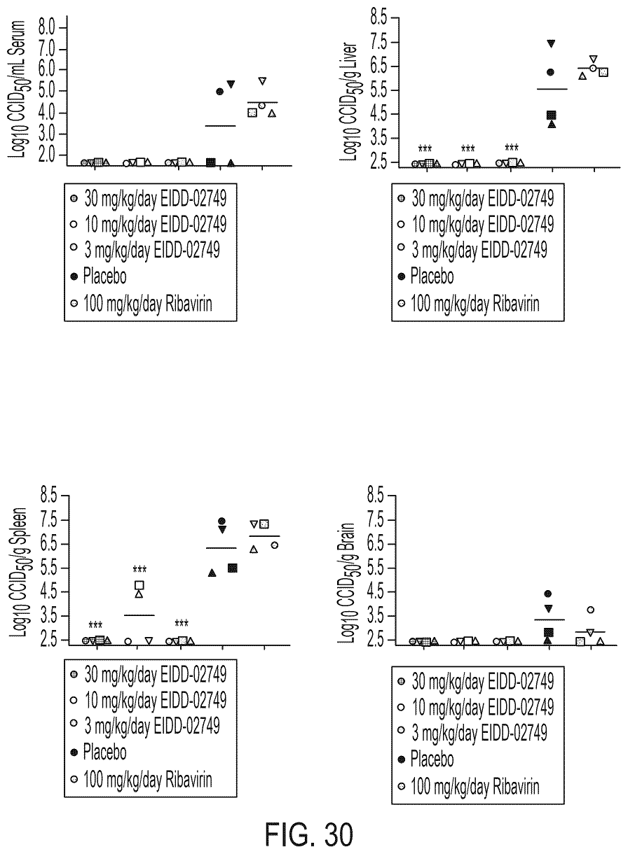

AG129 mice, inoculated with 500 CCID50 TCRV (strain TRVL 11573) i.p., received 3, 10, or 30 mg/kg/d EIDD-02749 PO for 14 days starting 2 hours before infection. Survival curves for the study are shown in FIG. 29 and viral titers measured on Day 9 are shown in FIG. 30. It is believed that an effective amount of a compound of Formulas XXIX-XXXIVb would provide similar results.

### Example 144. Therapeutic Efficacy of EIDD-2749 in a Mouse Model of Tacaribe Infection

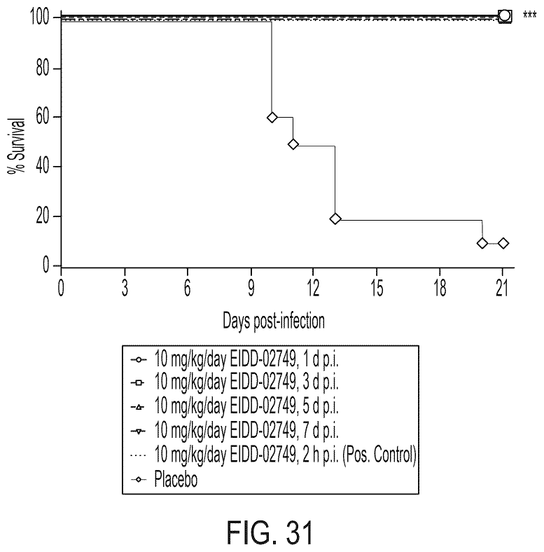

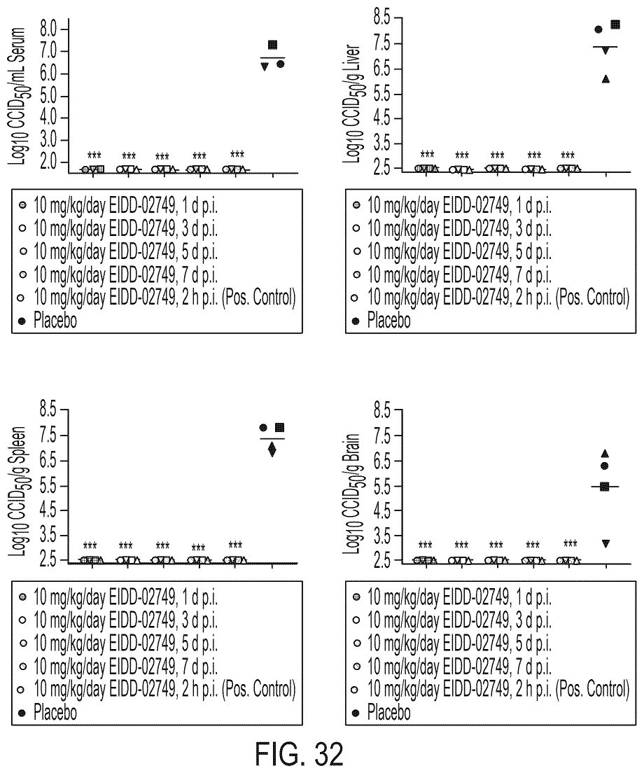

AG129 mice, inoculated with 500 CCID50 TCRV (strain TRVL 11573) i.p., received 10 mg/kg/d EIDD-02749 PO. Treatment started 2 hours, 1 Day, and 3 Days post infection and continued for 14 days, 5 Days post infection and continued for 12 days, and 7 Days post infection and continued for 10 days. Survival curves for the study are shown in FIG. 31 and viral titers measured on Day 9 are shown in FIG. 32. It is believed that an effective amount of a compound of Formulas XXIX-XXXIVb would provide similar results.

1. A compound having the Formula XXX: 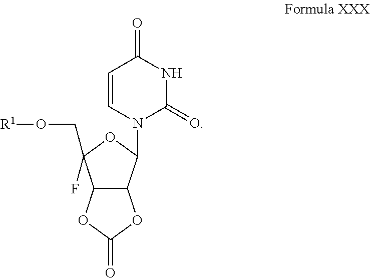
or a pharmaceutically acceptable salt thereof,
R¹ is a structure represented by a formula selected from: 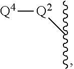
Q² is C(═O), O(C═O), S(C═O), NR⁴⁰(C═O), C(═S), O(C═S), S(C═S), or NR⁴⁰(C═S);
Q⁴ is C₅-C₈ cycloalkyl, C-C₁₂aryl, C₃-C₁₂heterocyclyl, or C₃-C₁₂heteroaryl; wherein Q⁴ can be optionally independently substituted with one or more, the same or different, R¹⁰;
R⁴⁰ is in each case independently selected from hydrogen and C₁-C₆alkyl, optionally substituted one more times by R¹⁰;
R¹⁰ is in each case independently deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and wherein R¹⁰ is optionally substituted with one or more, the same or different, R¹¹;
R¹¹ is in each case independently deuterium, hydroxy, azido, thiol, amino, cyano, halogen, alkyl, alkenyl, alkynyl, carbocyclyl, heterocarbocyclyl, aryl, heteroaryl, heterocyclyl, cycloalkyl, cycloalkenyl, alkoxy, carbocycloxy, heterocarbocycloxy, aryloxy, heteroaryloxy, heterocycloxy, cycloalkoxy, cycloalkenoxy, alkylamino, (alkyl)₂amino, carbocyclamino, heterocarbocyclamino, arylamino, heteroarylamino, heterocyclamino, cycloalkamino, cycloalkenamino, alkylthio, carbocyclylthio, heterocarbocyclylthio, arylthio, heteroarylthio, heterocyclylthio, cycloalkylthio, cycloalkenylthio, allenyl, sulfinyl, sulfamoyl, sulfonyl, lipid, nitro, polyethylene glycol, or carbonyl; and
lipid is independently a C₁₁-C₂₂alkyl, C₁₁-C₂₂alkoxy, or aryl substituted with an C₆-C₁₈ alkyl group.

2. The compound of claim 1, wherein Q² is C(═O).

3. The compound of claim 1, wherein Q² is C(═O), O(C═O), C(═S), or O(C═S).

4. The compound of claim 1, wherein Q⁴ is C₆-C₁₂aryl or C₃-C₁₂heteroaryl.

5. The compound of claim 1, wherein R¹ is: 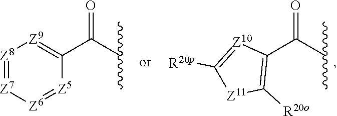
wherein
Z⁵ is selected from N and C—R^{20e};
Z⁶ is selected from N and C—R^{20f};
Z⁷ is selected from N and C—R^{20g};
Z⁸ is selected from N and C—R^{20h};
Z⁹ is selected from N and C—R²⁰ⁱ;
provided that no more than three of Z⁵, Z⁶, Z⁷, Z⁸, and Z⁹ are N;
R^{20e}, R^{20f}, R^{20g}, R^{20h}, R²⁰ⁱ, R^{20o}, and R^{20p} are each independently selected from hydrogen, deuterium, hydroxyl, amino, cyano, halogen, C₁-C₆ alkyl, OC₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl or lipid; wherein said alkyl groups are optionally independently substituted with one or more, the same or different, R¹⁰;
wherein two of R^{20e}, R^{20f}, R^{20g}, R^{20h}, and R²⁰ⁱ may together form a ring, for example an optionally substituted 5- to 7-membered cycloalkyl or heterocyclyl ring;
Z¹⁰ is selected from N and C—R^{20j};
Z¹¹ is selected from N—R^{20k} and C—R^{20l}R^{20m};
R^{20j}, R^{20l}, and R^{20m} are each independently selected from hydrogen, deuterium, hydroxyl, amino, cyano, halogen, C₁-C₆ alkyl, OC₁-C₆ alkyl, (C═O)C₁-C₆ alkyl, (C═O)NR⁴⁰C₁-C₆ alkyl, (C═O)N(C₁-C₆ alkyl)₂, (C═O)OC₁-C₆ alkyl, and lipid; wherein said alkyl groups are optionally independently substituted with one or more, the same or different, R¹⁰; wherein any two of more of R^{20j}, R^{20k}, R^{20l}, R^{20m}, R^{20o}, and R^{20p} may together form a ring;
R^{20k} is hydrogen or C₁-C₆ alkyl optionally independently substituted with one or more, the same or different, R¹⁰; and
R⁴⁰ is in each case independently selected from hydrogen and C₁-C₆alkyl, optionally substituted one or more times by R¹⁰.

6. The compound of claim 5, wherein R¹ is:

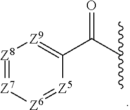

7. The compound of claim 5, having the formula:

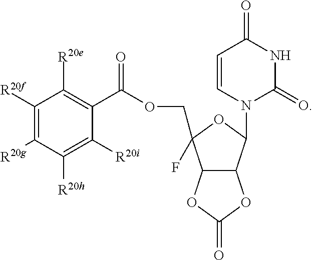

8. The compound of claim 7, wherein R^{20e}, R^{20f}, R^{20g}, R^{20h}, and R²⁰ⁱ are independently selected from hydrogen, deuterium, C₁-C₃alkyl, C₁-C₃alkoxy, C₁-C₃haloalkyl, C₁-C₃haloalkoxy, hydroxyl, amino, cyano, F, Cl, Br, and I, wherein at least one of R^{20e}, R^{20f}, R^{20g}, R^{20h}, and R²⁰ⁱ is not hydrogen.

9. The compound of claim 5, wherein R^{20e}, R^{20f}, R^{20g}, R^{20h}, and R²⁰ⁱ are independently selected hydrogen, C₁-C₃alkyl, C₁-C₃alkoxy, C₁-C₃haloalkyl, C₁-C₃haloalkoxy, and halogen, wherein at least one of R^{20e}, R^{20f}, R^{20g}, R^{20h}, and R²⁰ⁱ is not hydrogen.

10. The compound of claim 7, wherein R^{20e}, R^{20f}, R^{20g}, R^{20h}, and R²⁰ⁱ are independently selected from hydrogen, CH₃, OCH₃, CF₃, OCF₃, and halogen.

11. The compound of claim 10, wherein at least one of R^{20e}, R^{20f}, R^{20g}, R^{20h}, and R²⁰ⁱ is not hydrogen.

12. The compound of claim 11, wherein at least one of R^{20e}, R^{20f}, R^{20g}, R^{20h}, and R²⁰ⁱ is halogen.

13. The compound of claim 7, wherein one or two of R^{20e}, R^{20f}, R^{20g}, R^{20h}, and R²⁰ⁱ is halogen and the others are hydrogen.

14. The compound of claim 1, wherein Q⁴ is: 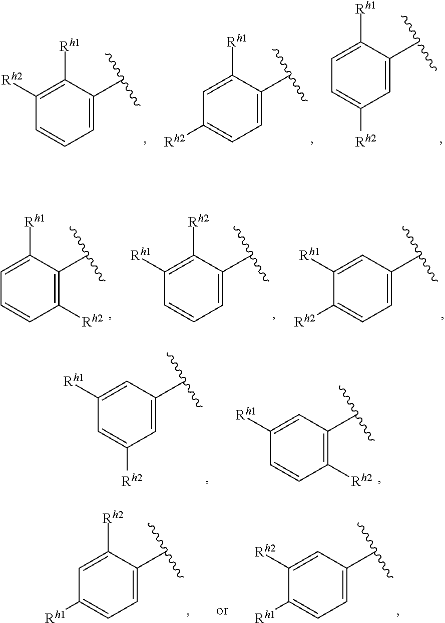
wherein R^{h1} is CH₃, OCH₃, CF₃, OCF₃, I, Cl or F, and R^{h2} is H, CH₃, OCH₃, CF₃, OCF₃, I, Cl, or F.

15. The compound of claim 1, wherein the compound is selected from:

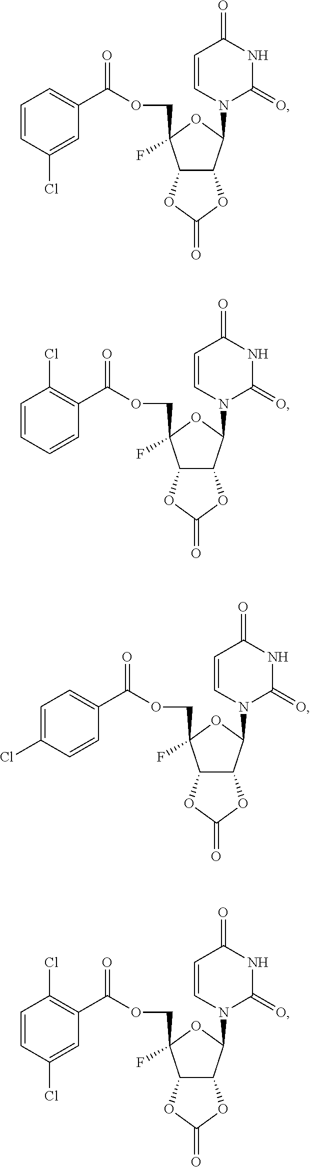 [SMILES: O=C1O[C@H]2[C@H](n3ccc(=O)[nH]c3=O)O[C@](F)(COC(=O)c3cccc(Cl)c3)[C@H]2O1 ; O=C1O[C@H]2[C@H](n3ccc(=O)[nH]c3=O)O[C@](F)(COC(=O)c3ccccc3Cl)[C@H]2O1 ; O=C1O[C@H]2[C@H](n3ccc(=O)[nH]c3=O)O[C@](F)(COC(=O)c3ccc(Cl)cc3)[C@H]2O1 ; O=C1O[C@H]2[C@H](n3ccc(=O)[nH]c3=O)O[C@](F)(COC(=O)c3cc(Cl)ccc3Cl)[C@H]2O1]

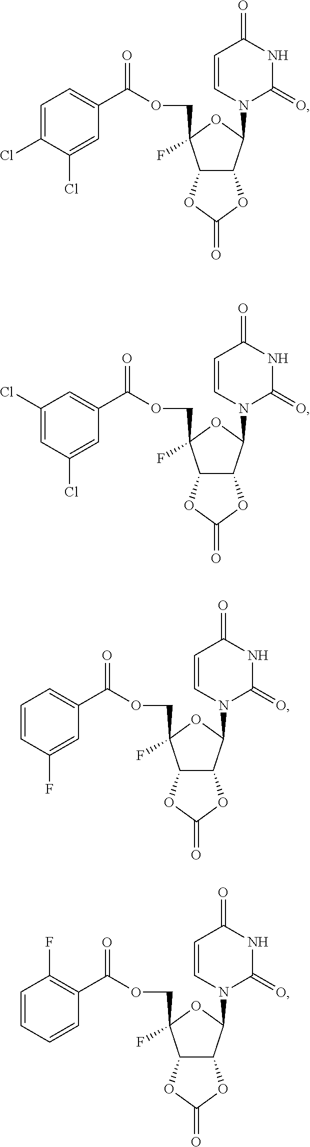

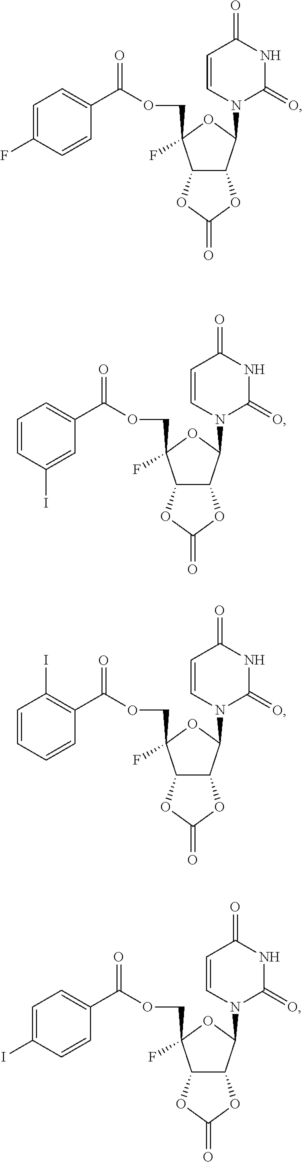 [SMILES: O=C1O[C@H]2[C@H](n3ccc(=O)[nH]c3=O)O[C@](F)(COC(=O)c3ccc(F)cc3)[C@H]2O1 ; O=C1O[C@H]2[C@H](n3ccc(=O)[nH]c3=O)O[C@](F)(COC(=O)c3cccc(I)c3)[C@H]2O1 ; O=C1O[C@H]2[C@H](n3ccc(=O)[nH]c3=O)O[C@](F)(COC(=O)c3ccccc3I)[C@H]2O1 ; O=C1O[C@H]2[C@H](n3ccc(=O)[nH]c3=O)O[C@](F)(COC(=O)c3ccc(I)cc3)[C@H]2O1]

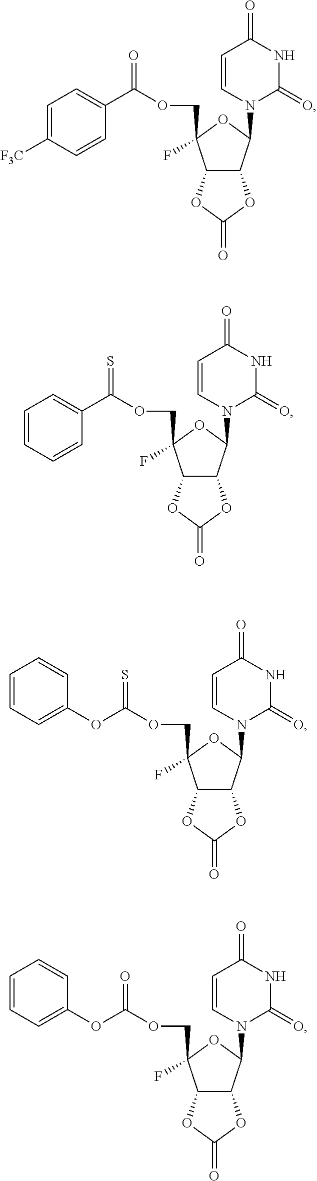 [SMILES: O=C1O[C@H]2[C@H](n3ccc(=O)[nH]c3=O)O[C@](F)(COC(=O)c3ccc(C(F)(F)F)cc3)[C@H]2O1 ; O=C1O[C@H]2[C@H](n3ccc(=O)[nH]c3=O)O[C@](F)(COC(=S)c3ccccc3)[C@H]2O1 ; O=C1O[C@H]2[C@H](n3ccc(=O)[nH]c3=O)O[C@](F)(COC(=S)Oc3ccccc3)[C@H]2O1 ; O=C(OC[C@@]1(F)O[C@@H](n2ccc(=O)[nH]c2=O)[C@@H]2OC(=O)O[C@@H]21)Oc1ccccc1]

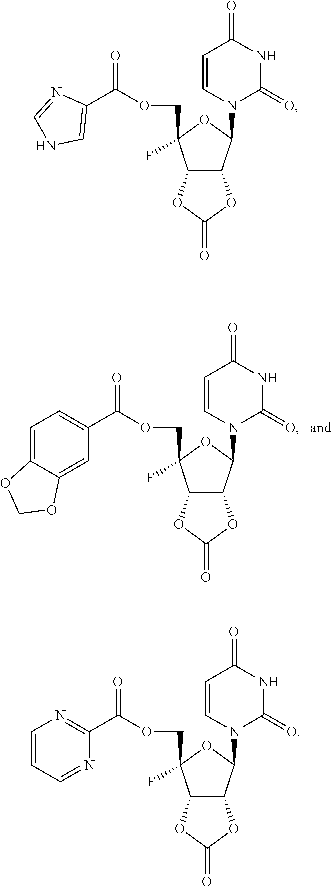 [SMILES: O=C1O[C@H]2[C@H](n3ccc(=O)[nH]c3=O)O[C@](F)(COC(=O)c3c[nH]cn3)[C@H]2O1 ; O=C1O[C@H]2[C@H](n3ccc(=O)[nH]c3=O)O[C@](F)(COC(=O)c3ccc4c(c3)OCO4)[C@H]2O1 ; "and" ; O=C1O[C@H]2[C@H](n3ccc(=O)[nH]c3=O)O[C@](F)(COC(=O)c3ncccn3)[C@H]2O1]

16. The compound of claim 1, having the formula:

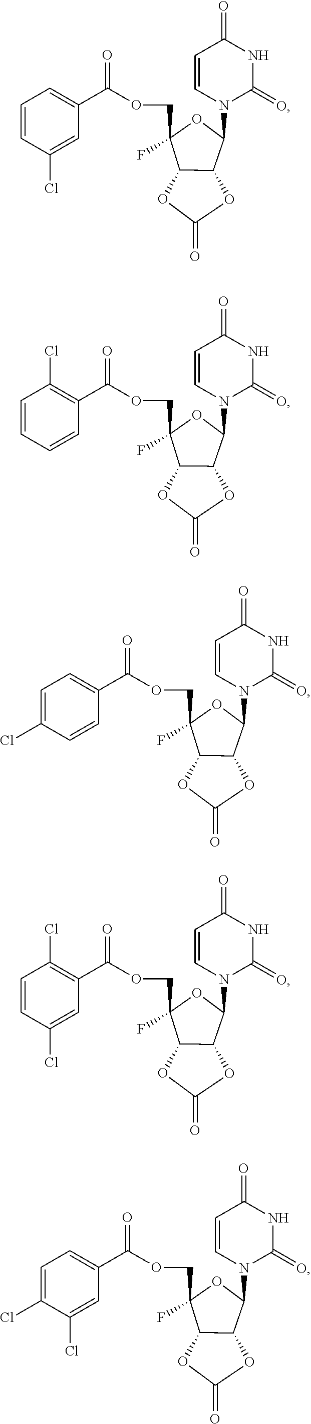

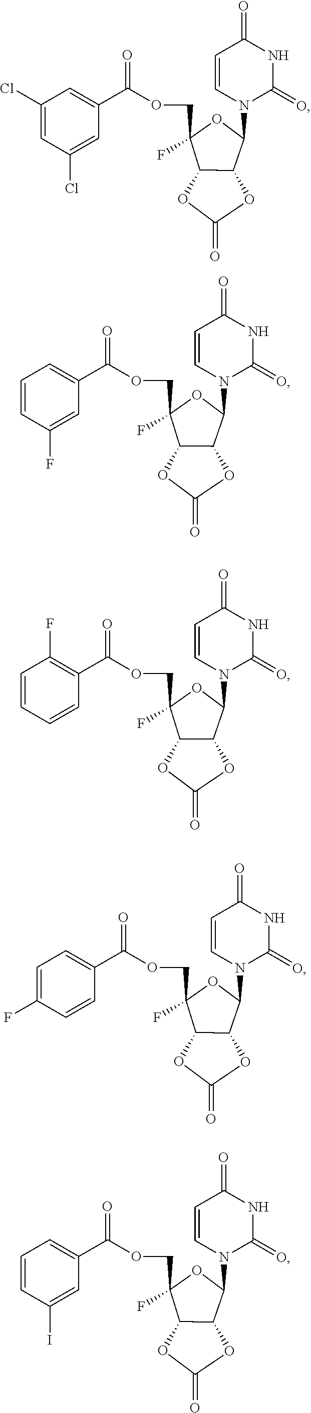

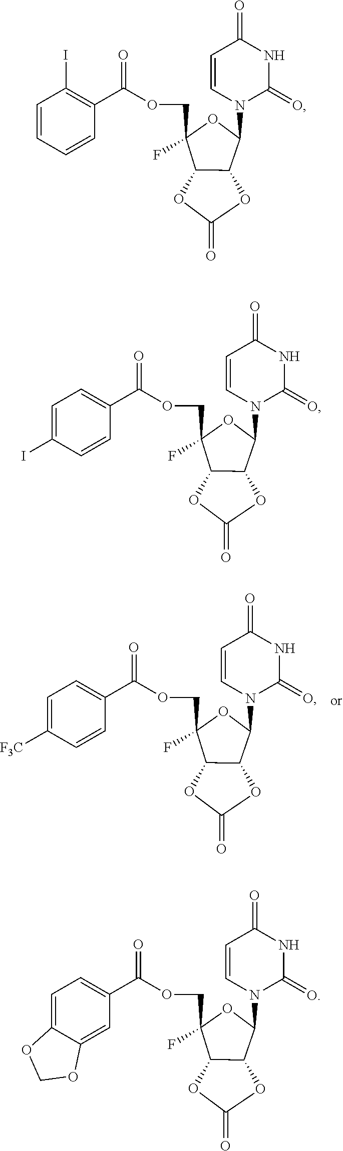 [SMILES: O=C1O[C@H]2[C@H](n3ccc(=O)[nH]c3=O)O[C@](F)(COC(=O)c3ccccc3I)[C@H]2O1 ; O=C1O[C@H]2[C@H](n3ccc(=O)[nH]c3=O)O[C@](F)(COC(=O)c3ccc(I)cc3)[C@H]2O1 ; O=C1O[C@H]2[C@H](n3ccc(=O)[nH]c3=O)O[C@](F)(COC(=O)c3ccc(C(F)(F)F)cc3)[C@H]2O1 ; O=C1O[C@H]2[C@H](n3ccc(=O)[nH]c3=O)O[C@](F)(COC(=O)c3ccc4c(c3)OCO4)[C@H]2O1]

17. The compound of claim 1, having the formula:

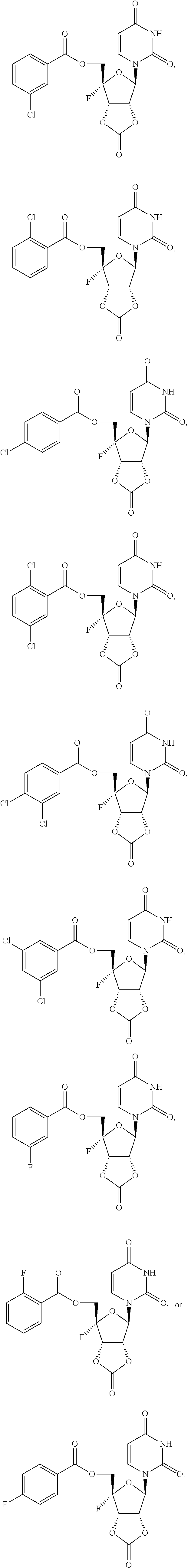

18. The compound of claim 1, having the formula:

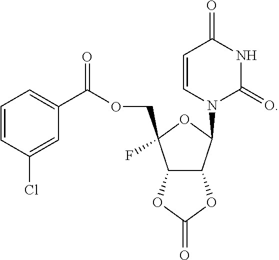 [SMILES: O=C1O[C@H]2[C@H](n3ccc(=O)[nH]c3=O)O[C@](F)(COC(=O)c3cccc(Cl)c3)[C@H]2O1]

19. A method of treating or preventing a viral infection in a subject in need thereof comprising administering in effective amount of the compound of claim 1 to the subject.

20. The method of claim 19, wherein the viral infection comprises an infection with arterivirus, arenavirus, bunyavirus, calcivirus, coronavirus, filovirus, orbovirus, orthomyxovirus, paramyxovirus, picornavirus, or togovirus.

21. The method of claim 20, wherein the picornavirus infection selected from infection with aphthovirus, cardiovirus, enterovirus, echovirus, coxsackievirus, heptovirus, kobuvirus, parechovirus, rhinovirus, and teschovirus,
wherein the togovirus infection is infection with an alphavirus selected from Chikungunya virus, Sindbis virus, Semliki Forest virus, Western equine encephalitis virus, Eastern Getah virus, Everglades virus, Venezuelan equine encephalitis virus, Ross River virus, Barmah Forest virus and Aura virus,
wherein the filovirus is selected from Marburg virus and Ebola virus,
wherein the orthomyxovirus is an influenza virus,
wherein the paramyxovirus is selected from Sendai virus, Hendra virus, Nipah virus, and parainfluenza virus,
wherein the bunyavirus is selected from Rift Valley fever virus, Punta Toro virus, LaCrosse virus, Maporal virus, Heartland virus, and Severe Fever Thrombocytopenia Syndrome virus, and
wherein the arenavirus is selected from Tacaribe virus, Pichinde virus, Junin virus, Lassa fever virus, and Lymphocytic Choriomeningitis virus.

22. The method of claim 20, wherein the coronavirus is selected from coronavirus is selected from SARS-CoV, HCoV-229E, HCoV-NL63, HCoV-OC43, bat SARS-like CoV, Middle East Respiratory Syndrome coronavirus (MERS), turkey coronavirus, chicken coronavirus, feline coronavirus and canine coronavirus.

23. The method of claim 20, wherein the coronavirus is a SARS-CoV2 virus.

24. The method of claim 21, wherein the influenza virus is selected from influenza A virus, influenza B virus, influenza C virus, and avian influenza virus.

25. The method of claim 21, wherein the influenza virus is an influenza virus subtype selected from H1N1 virus, H2N2 virus, H3N2 virus, H5N1 virus, H7N7 virus, H1N2 virus, H9N2 virus, H7N2 virus, H7N3 virus, H10N7 virus, H5N1 (low path) virus, H5N1 (high path) virus, H5N2 virus, H7N1 virus, H7N7 virus and H9N2 virus.

26. The method of claim 19, wherein the method further comprises administering a second antiviral agent to the subject.

27. The method of claim 19, wherein the subject is administered a loading dose of the compound in a first treatment period; and the subject is administered a treatment dose of the compound in a second treatment period following the first treatment period.

28. The method of claim 27, wherein the first treatment period is 1-5 days following diagnosis of the viral infection or presentation for preventing the viral infection.

29. A pharmaceutical composition comprising a pharmaceutically acceptable excipient and the compound of claim 1.

30. The pharmaceutical composition of claim 29, wherein the composition is an orally administrable composition.
